(19) United States
(12) Patent Application Publication
(10) Pub. No.: US 2025/0197407 A1
(43) Pub. Date: Jun. 19, 2025

(54) TRICYCLIC PHTHALAZINES AND DERIVATIVES AS SOS1 INHIBITORS

(71) Applicant: Jazz Pharmaceuticals Ireland Limited, Dublin (IE)

(72) Inventors: Clifford David JONES, Macclesfield (GB); Gayle DOUGLAS, Macclesfield (GB); Robin Charles HUMPHREYS, Dillon, CO (US); Camille GIGNOUX, Macclesfield (GB)

(21) Appl. No.: 18/849,340

(22) PCT Filed: Mar. 21, 2023
(86) PCT No.: PCT/EP2023/057247

§ 371 Date: Sep. 20, 2024

(30) Foreign Application Priority Data
Mar. 22, 2022 (GB) 2203976.2

(51) Int. Cl. C07D 487/04; A61K 31/5025; A61K 31/506; A61K 31/5383; A61K 31/553; C07D 491/20; C07D 498/04

(52) U.S. Cl. CPC C07D 487/04; A61K 31/5025; A61K 31/506; A61K 31/5383; A61K 31/553; C07D 491/20; C07D 498/04

(57) ABSTRACT

The present disclosure provides compounds that are inhibitors of SOS1, pharmaceutical compositions thereof, and methods of treating oncological diseases using the compounds and compositions disclosed herein.

### CROSS-REFERENCE TO RELATED APPLICATIONS

This application claims the benefit of GB Provisional Application No. 2203976.2, filed Mar. 22, 2022, which is incorporated by reference herein in its entirety.

### BACKGROUND

RAS proteins are a family of GTPases including KRAS (Kirsten rat sarcoma virus), NRAS (Neuroblastoma RAS viral oncogene homolog), HRAS (Harvey Rat sarcoma virus) and their respective mutants, that in cells exist in either GTP-bound or GDP-bound states. RAS proteins are critical signal transduction regulators that regulate cell proliferation, differentiation, migration and survival in different cell types. They play an important role in human cancer, with RAS oncogenic mutations identified in 20-30% of all human tumours, and for example are recognised as tumorigenic drivers in lung, colorectal and pancreatic cancers (Malumbres et al., 2001 Nature Reviews Cancer, 322-331; Pylayeva-Gupta et al., 2011 Nature Reviews Cancer, 761-774).

Acting as molecular switches, RAS proteins cycle between an active (GTP-bound) and an inactive (GDP-bound) state. Both GTPase activating proteins (GAPs) and guanine nucleotide exchange factors (GEFs) tightly regulate the activity status of RAS proteins. GAPs, such as NF1, deactivate RAS-GTP by stimulating the intrinsic GTPase catalytic activity of RAS proteins, leading to the hydrolysis and release of the gamma-phosphate of the bound GTP, resulting in inactive GDP-bound RAS protein. Binding of GEFs, such as SOS (Son of Sevenless) activate RAS proteins by stimulating the release of GDP thereby enabling the subsequent binding of the more abundant GTP, resulting in active GTP-bound RAS protein. Activated RAS proteins can signal through several downstream effector pathways, such as the RAF-MEK-ERK or Pi3K-Akt pathways. Cancer-associated mutations in RAS proteins suppress their ability to hydrolyse bound-GTP, even in presence of GAPs, leading to increased levels of active GTP-bound mutated RAS proteins (McCormick et al., 2015 Expert Opin. Ther. Targets, 19(4), 451-454). This in turn results in persistent activation of effector pathways downstream of RAS proteins.

The most widely studied RAS-GEF is the protein SOS, for which 2 human isoforms are known (SOS1 and SOS2). SOS1 and SOS2 both share 70% sequence similarity, with around 80% in the catalytic domain, but are both involved in different protein-protein interaction with RAS. Most studies suggest a dominant functional role of SOS1 over SOS2 in various physiological and pathological contexts (Baltanas et al., 2020 BBA Reviews on Cancer). SOS1 is a large multidomain protein of 1333 amino acids, consisting of 2 tandem N-terminal histone domains (HD) followed by a Dbl homology domain (DH), a Pleckstrin domain (PH), a helical linker (HL), a RAS exchange domain (REM), a CDC25 domain and a C-terminal proline rich domain (PR). The REM and CDC25 domains form the catalytic site involved in the nucleotide exchange activity on GDP-bound RAS (Kim et al., 1998 Oncogene 2597-2607). SOS1 also has an allosteric site, located between the CDC25 and the REM domains, that binds GTP-bound RAS proteins resulting in a further increase in the catalytic GEF function of SOS1 (Freedman et al., 2006 Proc. Natl. Acad. Sci. USA 16692-16697).

SOS1 has been shown to play a role in mutant KRAS activation and oncogenic signaling (Jeng et al., 2012 Nat. Commun., 3:1168). Oncogenic mutant KRAS activates wild-type (WT) RAS proteins through allosteric stimulation of SOS1 and this SOS1-mediated cross-activation of WT-RAS proteins contributes to cancer cell proliferation. Published data also indicates that SOS1 is involved in the activation of RAS protein signaling in cancer through mechanisms other than RAS mutations. The adaptor protein Grb2 associates with SOS1 via the binding of the Grb2 SH3 domains to the PR region of SOS1, and the complex becomes recruited to phosphorylated receptor tyrosine kinases (RTKs), for example EGFR or ALK through binding of the SH2 domains of Grb2 (Pierre et al., 2011 Biochem. Pharmacol., 82(9) 1049-1056). The SOS1-Grb2 complex also interacts with the oncoprotein Bcr-Abl, which is found in chronic myelogenous leukaemia (Kardinal et a., 2001 Blood, 98(6) 1773-1781). Other activated cell surface receptors like T-cell receptor, B-cell receptor and monocyte colony-stimulating factor receptor can recruit SOS1 to the plasma membrane, resulting in RAS-family protein activation (Salojin et al., 2000 J. Biol. Chem., 275(8) 5966-5975). SOS1 mutations in cancer are rare but can be present in many sporadic tumours including lung adenocarcinoma, urothelial bladder cancer and cutaneous melanoma. Furthermore, SOS1 mutations are also found in RASopathies such as Noonan syndrome and hereditary gingival fibromatosis (Baltanas et al., 2020 BBA Reviews on Cancer). In addition, SOS1 acts as GEF for the GTPase RAC, a member of the Rho subfamily of small GTPases, which is involved in angiogenesis and metastasis (Bid et al., 2013 Mol. Cancer Ther., 12(10) 1925-1934), although this is through SOS1 protein domains (PH-DH domains) distinct from those involved in RAS protein activation (REM-CDC25 domains).

The homolog SOS2 also acts as a GEF for RAS and RAC proteins (Pierre et al., 2011 Biochem. Pharmacol., 82(9) 1049-1056). Studies have showed that SOS2 is completely dispensable for mouse development, since SOS2 knockout mice survive to adulthood and were found to be viable and fertile, whereas SOS1 germline-null animals die during mid-gestation (Esteban et al., 2000 Mol. Cell. Biol., 20(17) 6410-6413; Qian et al., 2000 EMBO J., 19(4) 642-654). The systemic conditional knockout of SOS1 in adult mice demonstrated that SOS1 loss in adults is viable, whereas the equivalent SOS1/2 double knockout adult mice die precociously. This suggests functional redundancy in adults between SOS1 and SOS2 for lymphopoiesis, homeostasis and survival (Baltanas et al., 2013 Mol. Cell. Biol., 2013 33(22) 4562-4578). Selective inhibition of SOS1 functions over SOS2 may therefore represent a safe and viable approach for targeting RAS-driven tumors and pathologies.

Due to its role in the RAS protein mediated signaling pathways, SOS1 is an attractive target for cancer therapy. Recently, small molecules which selectively bind SOS1 and prevent its protein-protein interaction with RAS proteins have been reported. These compounds attenuate or eliminate the downstream effector events of RAS-mediated pathways e.g., ERK phosphorylation (Hillig et al., 2019 Proc. Natl. Acad. Sci. USA, 116(7) 2551-2560; Hofmann et al., 2020 Cancer Discovery, 142-157). In addition, several patent applications related to SOS1 inhibitors are published: WO2004003152, WO2016077793, WO2018115380, WO2018172250, WO2019122129, WO2019201848, WO2020180768, WO2020180770, WO2021092115, WO2021105960, WO2021124429, WO2021130731, WO2021173524.

### SUMMARY

In one aspect, the present disclosure provides a compound of Formula (I):

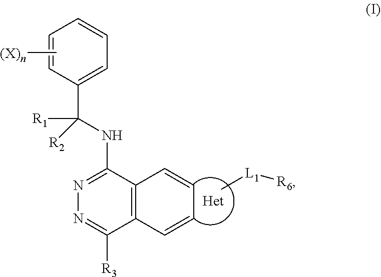

or a pharmaceutically acceptable salt thereof,
wherein:
X is independently halogen, alkyl, alkoxy, amino, amido, nitrile, acyl, cycloalkyl, heterocyclyl, or heteroaryl, and n is an integer from 1-5, and/or two X groups together with the atoms to which they are attached form a heterocyclyl or heteroaryl ring;
R₁ and R₂ are each independently hydrogen, alkyl, or R₁ and R₂ together with the atom to which they are attached form a cycloalkyl or heterocyclyl, wherein at least one of R₁ and R₂ is not hydrogen; and
R₃ is hydrogen, alkyl, —(C═O)—OR_{A}, —(C═O)—N(R_{A})₂, cycloalkyl, heterocyclyl, aryl or heteroaryl, wherein each R_{A} is independently hydrogen or alkyl;

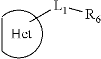

is a nitrogen-containing heterocyclyl substituted with L₁-R₆ and 0-6 substituents independently selected from R₈, R₉, R₁₀, and R₁₁; L₁ is absent, alkylene, alkenylene, or alkynylene;
R₆ is alkyl, —O-alkyl, cycloalkyl, or heterocyclyl;
R₈ and R₉ are each independently H, halogen, or alkyl, or an R₈ and R₉ together with the carbon atom to which they are attached form a C₃₋₆cycloalkyl, a 3- to 6-membered heterocyclyl, or a carbonyl;
R₁₀ is H, halogen, or -L₂-R₇, wherein L₂ is absent, alkylene, alkenylene, or alkynylene; and R₇ is H, alkyl, —O-alkyl, cycloalkyl, or heterocyclyl; and
R₁₁ is H, halogen, or alkyl, or an R₁₀ and R₁₁ together with the carbon atom to which they are attached form a C₃₋₆cycloalkyl, a 3- to 6-membered heterocyclyl, or a carbonyl.

In one aspect, the present disclosure provides a compound of Formula (Ia):

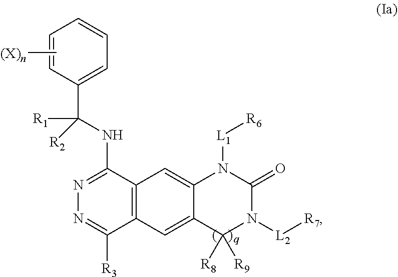

or a pharmaceutically acceptable salt thereof,
wherein:
X is independently halogen, alkyl, alkoxy, amino, amido, nitrile, acyl, cycloalkyl, heterocyclyl, or heteroaryl, and n is an integer from 1-5, and/or two X groups together with the atoms to which they are attached form a heterocyclyl or heteroaryl ring;
L₁ and L₂ are each independently absent, alkylene, alkenylene, or alkynylene;
R₁ and R₂ are each independently hydrogen, alkyl, or R₁ and R₂ together with the atom to which they are attached form a cycloalkyl or heterocyclyl, wherein at least one of R₁ and R₂ is not hydrogen; and
R₃ is hydrogen, alkyl, —(C═O)—OR_{A}, —(C═O)—N(R_{A})₂, cycloalkyl, heterocyclyl, aryl or heteroaryl, wherein each R_{A} is independently hydrogen or alkyl;
R₆ is alkyl, —O-alkyl, cycloalkyl, or heterocyclyl;
R₇ is H, alkyl, —O-alkyl, cycloalkyl, or heterocyclyl;
R₈ and R₉ are each independently H, F, or C₁₋₅alkyl, or an R₈ and R₉ together with the carbon atom to which they are attached form a C₃₋₆cycloalkyl, a 3- to 6-membered heterocyclyl, or a carbonyl; and
q is 0 or 1.

In one aspect, the present disclosure provides a compound of Formula (Ib):

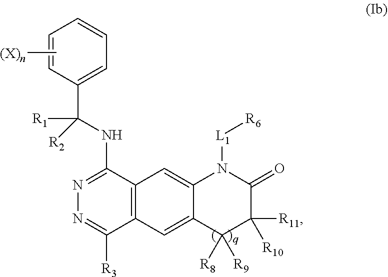

or a pharmaceutically acceptable salt thereof,
wherein:
X is independently halogen, alkyl, alkoxy, amino, amido, nitrile, acyl, cycloalkyl, heterocyclyl, or heteroaryl, and n is an integer from 1-5, and/or two X groups together with the atoms to which they are attached form a heterocyclyl or heteroaryl ring;
L₁ and L₂ are each independently absent, alkylene, alkenylene, or alkynylene;
R₁ and R₂ are each independently hydrogen, alkyl, or R₁ and R₂ together with the atom to which they are attached form a cycloalkyl or heterocyclyl, wherein at least one of R₁ and R₂ is not hydrogen; and
R₃ is hydrogen, alkyl, —(C═O)—OR_{A}, —(C═O)—N(R_{A})₂, cycloalkyl, heterocyclyl, aryl or heteroaryl, wherein each R_{A} is independently hydrogen or alkyl;
R₆ is alkyl, —O-alkyl, cycloalkyl, or heterocyclyl;
R₇ is H, alkyl, —O-alkyl, cycloalkyl, or heterocyclyl;
R₈ and R₉ are each independently H, F, or C₁₋₅alkyl, or an R₈ and R₉ together with the carbon atom to which they are attached form a C₃₋₆cycloalkyl, a 3- to 6-membered heterocyclyl, or a carbonyl;
R₁₀ is H, F, C₁₋₅alkyl, or -L₂-R₇;
R₁₁ is H, F, or C₁₋₅alkyl, or an R₁₀ and R₁₁ together with the carbon atom to which they are attached form a C₃₋₆cycloalkyl or a 3- to 6-membered heterocyclyl; and
q is 0 or 1.

In some embodiments of Formula (Ia) and Formula (Ib), q is 0. In some embodiments, q is 1.

In one aspect, the present disclosure provides a compound of Formula (Ic):

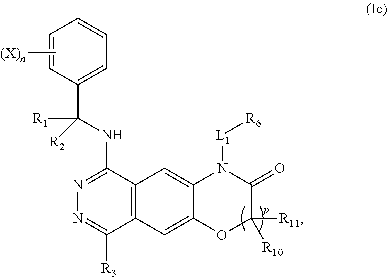

or a pharmaceutically acceptable salt thereof,
wherein:
X is independently halogen, alkyl, alkoxy, amino, amido, nitrile, acyl, cycloalkyl, heterocyclyl, or heteroaryl, and n is an integer from 1-5, and/or two X groups together with the atoms to which they are attached form a heterocyclyl or heteroaryl ring;
L₁ and L₂ are each independently absent, alkylene, alkenylene, or alkynylene;
R₁ and R₂ are each independently hydrogen, alkyl, or R₁ and R₂ together with the atom to which they are attached form a cycloalkyl or heterocyclyl, wherein at least one of R₁ and R₂ is not hydrogen; and
R₃ is hydrogen, alkyl, —(C═O)—OR_{A}, —(C═O)—N(R_{A})₂, cycloalkyl, heterocyclyl, aryl or heteroaryl, wherein each R_{A} is independently hydrogen or alkyl;
R₆ is alkyl, —O-alkyl, cycloalkyl, or heterocyclyl;
R₇ is H, alkyl, —O-alkyl, cycloalkyl, or heterocyclyl;
R₁₀ is H, F, C₁₋₅alkyl, or -L₂-R₇;
R₁₁ is H, F, or C₁₋₅alkyl, or an R₁₀ and R₁₁ together with the carbon atom to which they are attached form a C₃₋₆cycloalkyl, a 3- to 6-membered heterocyclyl, or a carbonyl; and
p is 1 or 2.

In one aspect, the present disclosure provides a compound of Formula (Ic-1):

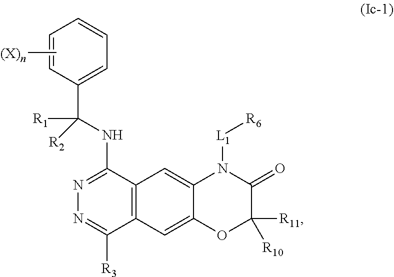

or a pharmaceutically acceptable salt thereof,
wherein:
X is independently halogen, alkyl, alkoxy, amino, amido, nitrile, acyl, cycloalkyl, heterocyclyl, or heteroaryl, and n is an integer from 1-5, and/or two X groups together with the atoms to which they are attached form a heterocyclyl or heteroaryl ring;
L₁ and L₂ are each independently absent, alkylene, alkenylene, or alkynylene;
R₁ and R₂ are each independently hydrogen, alkyl, or R₁ and R₂ together with the atom to which they are attached form a cycloalkyl or heterocyclyl, wherein at least one of R₁ and R₂ is not hydrogen; and
R₃ is hydrogen, alkyl, —(C═O)—OR_{A}, —(C═O)—N(R_{A})₂, cycloalkyl, heterocyclyl, aryl or heteroaryl, wherein each R_{A} is independently hydrogen or alkyl;
R₆ is alkyl, —O-alkyl, cycloalkyl, or heterocyclyl;
R₇ is H, alkyl, —O-alkyl, cycloalkyl, or heterocyclyl;
R₁₀ is H, F, C₁₋₅alkyl, or -L₂-R₇; and
R₁₁ is H, F, or C₁₋₅alkyl, or an R₁₀ and R₁₁ together with the carbon atom to which they are attached form a C₃₋₆cycloalkyl, a 3- to 6-membered heterocyclyl, or a carbonyl.

In some embodiments,

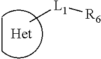

is:

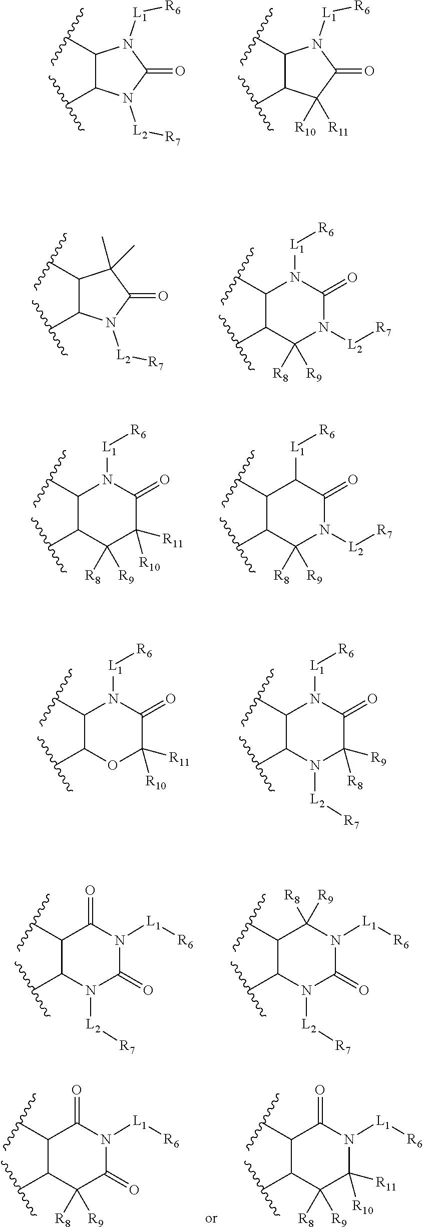

wherein
R₈ and R₉ are each independently H, F, or C₁₋₅alkyl, or an R₈ and R₉ together with the carbon atom to which they are attached form a C₃₋₆cycloalkyl or a 3- to 6-membered heterocyclyl;
R₁₀ is H, F, C₁₋₅alkyl, or -L₂-R₇; and
R₁₁ is H, F, or C₁₋₅alkyl, or an R₁₀ and R₁₁ together with the carbon atom to which they are attached form a C₃₋₆cycloalkyl, a 3- to 6-membered heterocyclyl, or a carbonyl.

In some embodiments, L₁ and L₂ are each independently absent or C₁₋₅alkylene. In some embodiments, L₁ is C₁₋₅alkylene. In some embodiments, the C₁₋₅alkylene is —CH₂— or —CH₂CH₂—. In some embodiments, L₁ and L₂ are absent. In some embodiments, L₂ is absent. In some embodiments, L₁ is C₁₋₅alkylene and L₂ is absent.

In some embodiments, X is independently halogen, alkyl, alkoxy, amino, amido, nitrile, or acyl, and n is an integer from 1-5, and/or two X groups together with the atoms to which they are attached form a heterocyclyl or heteroaryl ring. In some embodiments, each X is independently halogen, alkyl, alkoxy or amino. In some embodiments, each X is independently halogen, alkyl, or amino. In some embodiments, the alkyl is substituted with one or more halogen, hydroxyl, alkoxy, amino, or combination thereof. In some embodiments, each X is independently halogen, haloalkyl, haloalkoxy, or amino. In some embodiments, each X is independently halogen, haloalkyl or amino. In some embodiments, the haloalkyl is a fluoroalkyl. In some embodiments, each X is independently a fluoroalkyl, fluoroalkoxy, F or —NH₂. In some embodiments, each X is independently —CH₂F, —CHF₂, —CF₃, —CF₂CH₂OH, —CF₂C(CH₃)₂OH, —CF₂C(CH₃)(CH₂OMe)OH, —CF₂C(CH₃)(CH₂NHMe)OH, —CF₂C(CH₃)(CH₂NMe₂)OH, —CF₂CH₂NH₂, —CF₂CH₂NMe₂, —CF₂C(CH₃)₂NH₂, —CF₂C(CH₃)₂NMe₂, F, or —NH₂. In some embodiments, each X is independently —CH₂F, —CHF₂, —CF₃, —CF₂CH₂OH, —CF₂C(CH₃)₂OH, —CF₂C(CH₃)(CH₂OMe)OH, —CF₂C(CH₃)(CH₂NHMe)OH, —CF₂C(CH₃)(CH₂NMe₂)OH, —CF₂C(CH₃)₂NH₂, F, or —NH₂. In some embodiments, each X is independently —CF₂CH₃, —CF₂CH₂OH, —CF₂C(CH₃)₂OH, —CHF₂, —CF₃, F, or —NH₂.

In some embodiments

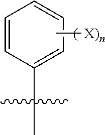

is:

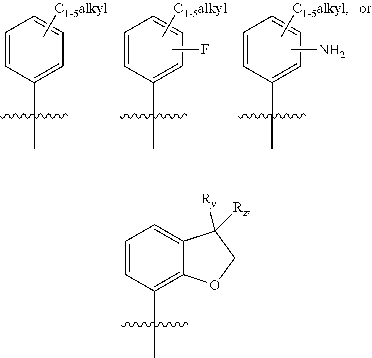

wherein R_{y} and R_{z} are each independently H, F, or alkyl, or an R_{y} and R_{z} together with the carbon atom to which they are attached form a C₃₋₆cycloalkyl. In some embodiments, X is a C₁₋₅haloalkyl. In some embodiments, X is a C₁_fluoroalkyl. In some embodiments, X is —CH₂F, —CHF₂, —CF₃, —CF₂CH₂OH, —CF₂CH₂OCH₃, —CF₂C(CH₃)₂OH, —CF₂C(CH₃)₂OCH₃, —CF₂C(CH₃)(CH₂OMe)OH, —CF₂C(CH₃)(CH₂NHMe)OH, —CF₂C(CH₃)(CH₂NMe₂)OH, —CF₂CH₂NH₂, —CF₂CH₂NHMe, —CF₂CH₂NMe₂, —CF₂C(CH₃)₂NH₂, —CF₂C(CH₃)₂NHMe, or —CF₂C(CH₃)₂NMe₂. In some embodiments, X is —CH₂F, —CHF₂, —CF₃, —CF₂CH₂OH, —CF₂C(CH₃)₂OH, —CF₂C(CH₃)(CH₂OMe)OH, —CF₂C(CH₃)(CH₂NHMe)OH, —CF₂C(CH₃)(CH₂NMe₂)OH, —CF₂CH₂NH₂, —CF₂CH₂NMe₂, —CF₂C(CH₃)₂NH₂, or —CF₂C(CH₃)₂NMe₂. In some embodiments, X is —CF₂CF₃, —CF₂CH₃, —CF₂CH₂OH, —CF₂C(CH₃)₂OH, —CHF₂, —CF₃, or —CH₂F. In some embodiments, X is —CF₂CH₂OH, —CF₂C(CH₃)₂OH, —CHF₂, or —CF₃.

In some embodiments

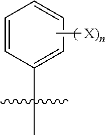

is:

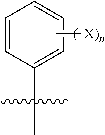

In some embodiments,

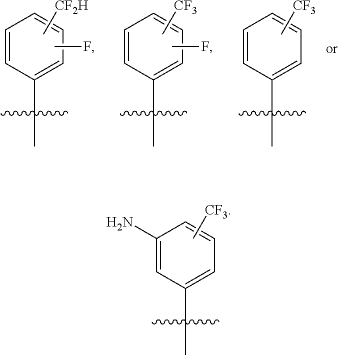

is:

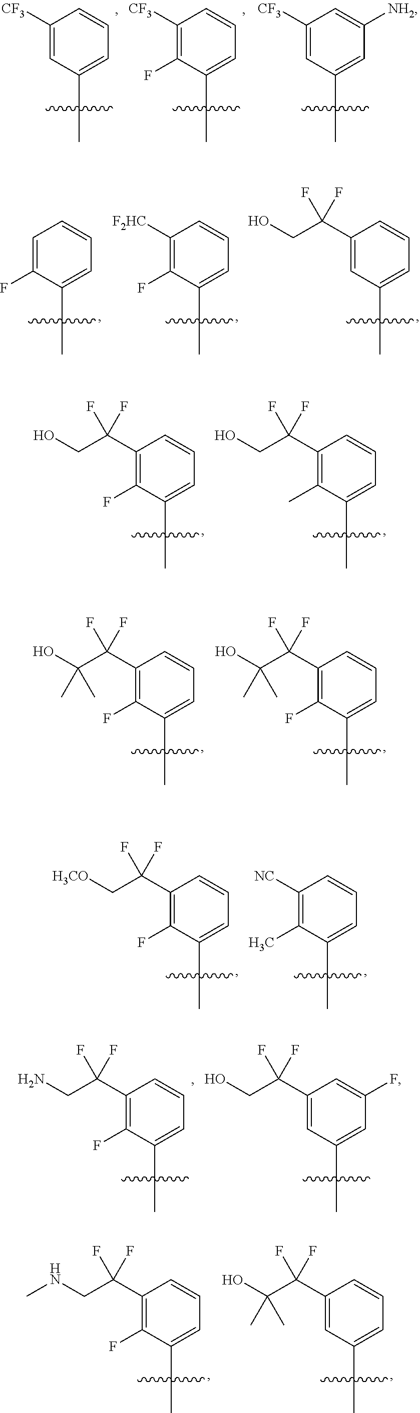

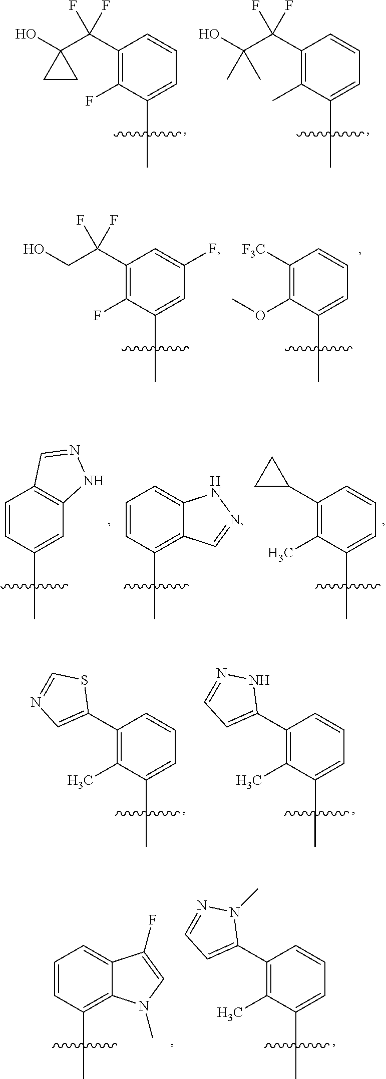

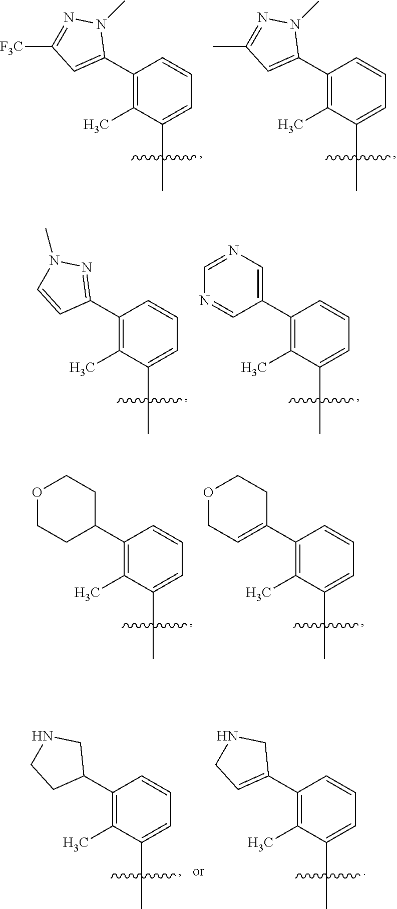

In some embodiments, n is 1 or 2. In some embodiments, n is 1. In some embodiments, n is 2.

In some embodiments, R₁ is alkyl and R₂ is H. In some embodiments, R₁ is C₁₋₅alkyl and R₂ is H. In some embodiments, R₁ is methyl and R₂ is H. In some embodiments, R₂ is alkyl and R₁ is H. In some embodiments, R₂ is C₁₋₅alkyl and R₁ is H. In some embodiments, R₂ is methyl and R₁ is H.

In some embodiments, R₃ is H, methyl, ethyl, isopropyl, n-propyl, —CH₂OH, —CH₂OCH₃, —CH₂N(CH₃)₂, —CH(OH)(CH₃)₂ or —CH₂(OH)CH₃. In some embodiments, R₃ is H, methyl, ethyl, isopropyl, n-propyl, —CH₂OH, —CH₂OCH₃, —CH₂N(CH₃)₂, —CH(OH)(CH₃)₂ or —CH₂(OH)CH₃. In some embodiments, R₃ is H or C₁₋₅alkyl. In some embodiments, R₃ is H or methyl. In some embodiments, R₃ is methyl or ethyl. In some embodiments, R₃ is methyl.

In some embodiments, R₆ is C₁₋₅alkyl, —O—C₁₋₅alkyl, C₃₋₆cycloalkyl, or 3- to 6-membered heterocyclyl. In some embodiments, R₆ is C₁₋₅alkyl. In some embodiments, R₆ is methyl, ethyl, isopropyl, tert-butyl, —CH₂CF₃, CH₂CF₂H, —CH(CH₃)CF₃, CH(CH₃)CF₂H, —C(CH₃)₂CF₃, or —C(CH₃)₂CF₂H. In some embodiments, R₆ is methyl, ethyl, or isopropyl. In some embodiments, R₆ is —O—C₁₋₅alkyl. In some embodiments, R₆ is —OCH₃ or —OCH₂CH₃. In some embodiments, R₆ is —OCH₃. In some embodiments, R₆ is 3- to 6-membered heterocyclyl. In some embodiments, R₆ is a 5- or 6-membered heterocyclyl having 1 or 2 heteroatoms selected from N, O, or S. In some embodiments, R₆ is a morpholinyl, piperazinyl, piperidinyl, pyrrolidinyl, azetidinyl, tetrahydropyranyl, or tetrahydrofuranyl. In some embodiments, R₆ is

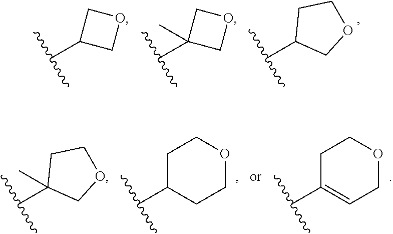

In some embodiments, R₆ is

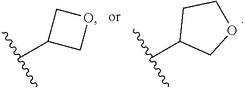 [SMILES: *C1COC1 ; "or" ; *C1CCOC1]

In some embodiments, R₆ is C₃₋₆cycloalkyl. In some embodiments, R₆ is

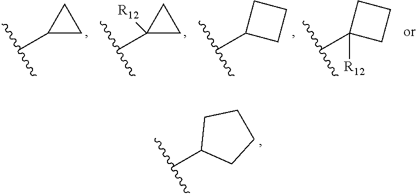

wherein R₁₂ is C₁₋₅alkyl. In some embodiments, R₆ is

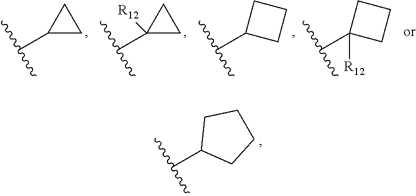

wherein R₁₂ is —CH₃, —CF₃ or —CF₂H. In some embodiments, R₆ is cyclopentyl. In some embodiments, R₆ is methyl, cyclopentyl or 3-tetrahydrofuranyl.

In some embodiments, R₇ is C₁₋₅alkyl, —O—C₁₋₅alkyl, C₃₋₅cycloalkyl, or 3- to 5-membered heterocyclyl. In some embodiments, R₇ is C₁₋₅alkyl. In some embodiments, R₇ is methyl, —O— methyl, or 3-tetrahydrofuran. In some embodiments, R₇ is methyl.

In some embodiments, R₆ is alkyl, —O-alkyl, cycloalkyl, or heterocyclyl and R₇ is H, alkyl, —O-alkyl, cycloalkyl, or heterocyclyl. In some embodiments, R₆ is alkyl, —O-alkyl, cycloalkyl, or heterocyclyl and R₇ is alkyl. In some embodiments, R₆ is alkyl, cycloalkyl, or heterocyclyl, and R₇ is alkyl. In some embodiments, R₆ is alkyl or cycloalkyl and R₇ is alkyl.

In some embodiments, R₈ and R₉ are each independently H, F, or C₁₋₅alkyl, or an R₈ and R₉ together with the carbon atom to which they are attached form a C₃₋₆cycloalkyl or a 3- to 6-membered heterocyclyl. In some embodiments, R₈ and R₉ are each independently H, halogen, or alkyl. In some embodiments, R₈ and R₉ are each independently H, F, or C₁₋₅alkyl. In some embodiments, the C₁₋₅alkyl is methyl, ethyl or isopropyl. In some embodiments, C₁₋₅alkyl is methyl or ethyl. In some embodiments, R₈ and R₉ are each H. In some embodiments, R₈ and R₉ are each F. In some embodiments, R₈ and R₉ are each C₁₋₅alkyl. In some embodiments, R₈ and R₉ are each methyl. In some embodiments, R₈ and R₉ are each ethyl.

In some embodiments, R₈ and R₉ together with the carbon atom to which they are attached form a C₃₋₆cycloalkyl or a 3- to 6-membered heterocyclyl. In some embodiments, R₈ and R₉ together with the carbon atom to which they are attached form a C₃₋₆cycloalkyl. In some embodiments, R₈ and R₉ together with the carbon atom to which they are attached form a cyclopropyl. In some embodiments, R₈ and R₉ together with the carbon atom to which they are attached form an azetidine, pyrrolidine, or piperidine. In some embodiments, R₈ and R₉ together with the carbon atom to which they are attached form a carbonyl.

In some embodiments, R₁₀ is H, halogen, or -L₂-R₇, wherein L₂ is absent, alkylene, alkenylene, or alkynylene; and R₇ is as defined herein. In some embodiments, R₇ is H, C₁₋₅alkyl, —O—C₁₋₅alkyl, C₄₋₆cycloalkyl, or 3- to 6-membered heterocyclyl.

In some embodiments, R₁ is H, halogen, or C₁₋₅alkyl. In some embodiments, R₁ is H, F, or C₁₋₅alkyl. In some embodiments, the C₁₋₅alkyl is methyl, ethyl or isopropyl. In some embodiments, C₁₋₅alkyl is methyl or ethyl. In some embodiments, R₁₁ is H. In some embodiments, R₁₁ is F. In some embodiments, R₁₁ is C₁₋₅alkyl. In some embodiments, R₁₁ is methyl. In some embodiments, R₁₁ is ethyl.

In some embodiments, R₁₁ is H, F, or C₁₋₅alkyl, or an R₁₀ and R₁₁ together with the carbon atom to which they are attached form a C₃₋₆cycloalkyl or a 3- to 6-membered heterocyclyl. In some embodiments, R₁₀ and R₁₁ together with the carbon atom to which they are attached form a C₃₋₆cycloalkyl or a 3- to 6-membered heterocyclyl. In some embodiments, R₁₀ and R₁₁ together with the carbon atom to which they are attached form a C₃₋₆cycloalkyl. In some embodiments, R₁₀ and R₁ together with the carbon atom to which they are attached form a cyclopropyl. In some embodiments, R₁₀ and R₁₁ together with the carbon atom to which they are attached form a carbonyl.

In some embodiments, R₁₀ and R₁₁ are each F. In some embodiments, R₁₀ and R₁₁ are each Me. In some embodiments, R₁₀ is -L₂-R₇ and R₁₁, when present, is H. In some embodiments, R₁₀ is -L₂-R₇ and R₁₁, when present, is Me.

In some embodiments, the compound of the present disclosure is:

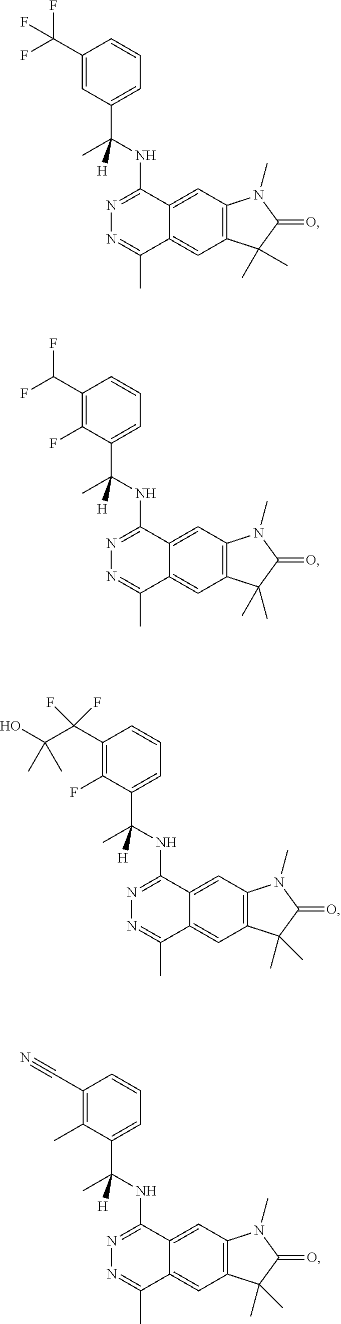 [SMILES: Cc1nnc(N[C@H](C)c2cccc(C(F)(F)F)c2)c2cc3c(cc12)C(C)(C)C(=O)N3C ; Cc1nnc(N[C@H](C)c2cccc(C(F)F)c2F)c2cc3c(cc12)C(C)(C)C(=O)N3C ; Cc1nnc(N[C@H](C)c2cccc(C(F)(F)C(C)(C)O)c2F)c2cc3c(cc12)C(C)(C)C(=O)N3C ; Cc1c(C#N)cccc1[C@@H](C)Nc1nnc(C)c2cc3c(cc12)N(C)C(=O)C3(C)C]

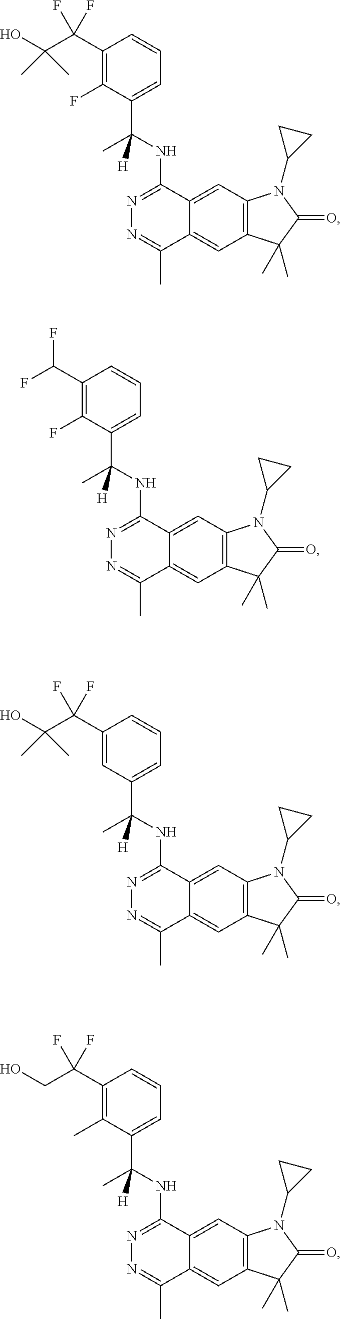 [SMILES: Cc1nnc(N[C@H](C)c2cccc(C(F)(F)C(C)(C)O)c2F)c2cc3c(cc12)C(C)(C)C(=O)N3C1CC1 ; Cc1nnc(N[C@H](C)c2cccc(C(F)F)c2F)c2cc3c(cc12)C(C)(C)C(=O)N3C1CC1 ; Cc1nnc(N[C@H](C)c2cccc(C(F)(F)C(C)(C)O)c2)c2cc3c(cc12)C(C)(C)C(=O)N3C1CC1 ; Cc1c([C@@H](C)Nc2nnc(C)c3cc4c(cc23)N(C2CC2)C(=O)C4(C)C)cccc1C(F)(F)CO]

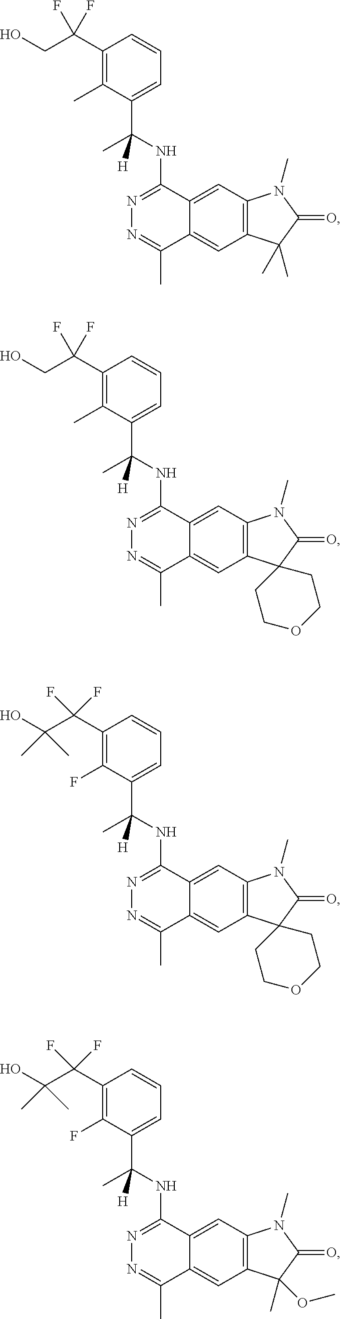 [SMILES: Cc1c([C@@H](C)Nc2nnc(C)c3cc4c(cc23)N(C)C(=O)C4(C)C)cccc1C(F)(F)CO ; Cc1c([C@@H](C)Nc2nnc(C)c3cc4c(cc23)N(C)C(=O)C42CCOCC2)cccc1C(F)(F)CO ; Cc1nnc(N[C@H](C)c2cccc(C(F)(F)C(C)(C)O)c2F)c2cc3c(cc12)C1(CCOCC1)C(=O)N3C ; COC1(C)C(=O)N(C)c2cc3c(N[C@H](C)c4cccc(C(F)(F)C(C)(C)O)c4F)nnc(C)c3cc21]

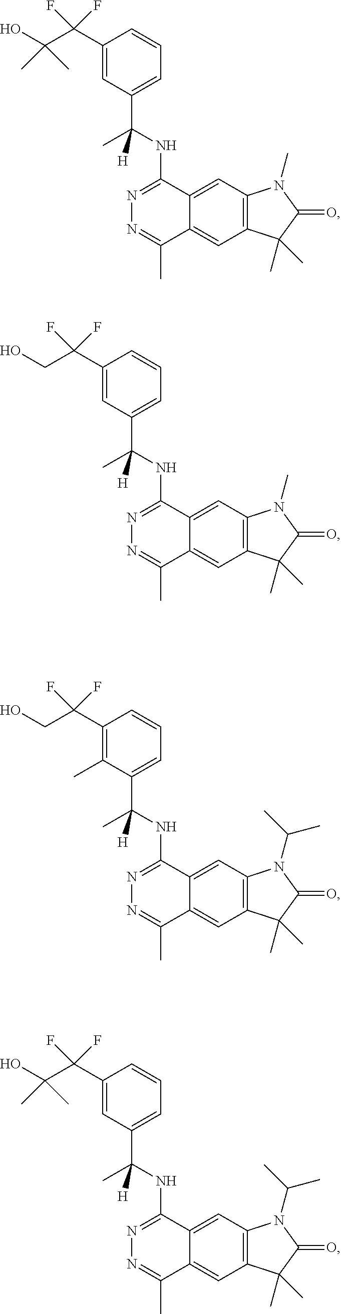 [SMILES: Cc1nnc(N[C@H](C)c2cccc(C(F)(F)C(C)(C)O)c2)c2cc3c(cc12)C(C)(C)C(=O)N3C ; Cc1nnc(N[C@H](C)c2cccc(C(F)(F)CO)c2)c2cc3c(cc12)C(C)(C)C(=O)N3C ; Cc1c([C@@H](C)Nc2nnc(C)c3cc4c(cc23)N(C(C)C)C(=O)C4(C)C)cccc1C(F)(F)CO ; Cc1nnc(N[C@H](C)c2cccc(C(F)(F)C(C)(C)O)c2)c2cc3c(cc12)C(C)(C)C(=O)N3C(C)C]

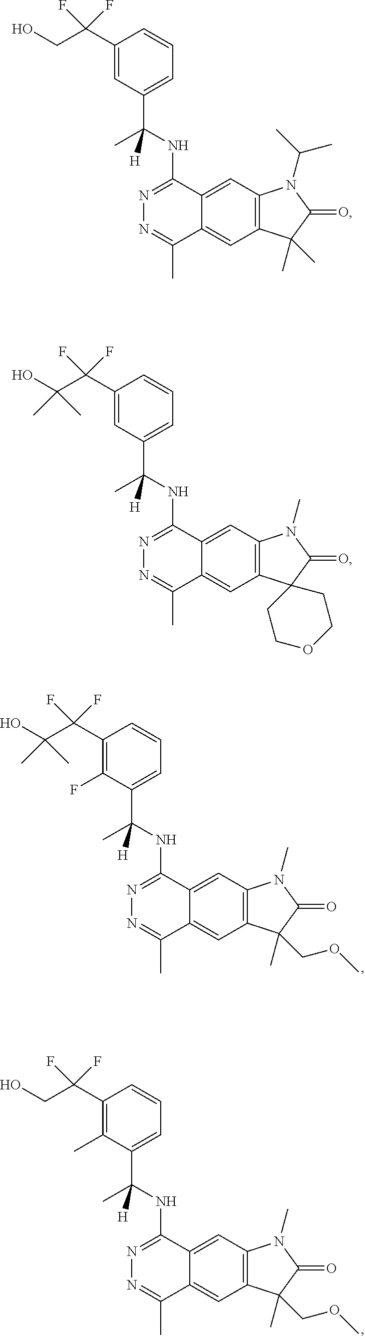 [SMILES: Cc1nnc(N[C@H](C)c2cccc(C(F)(F)CO)c2)c2cc3c(cc12)C(C)(C)C(=O)N3C(C)C ; Cc1nnc(N[C@H](C)c2cccc(C(F)(F)C(C)(C)O)c2)c2cc3c(cc12)C1(CCOCC1)C(=O)N3C ; COCC1(C)C(=O)N(C)c2cc3c(N[C@H](C)c4cccc(C(F)(F)C(C)(C)O)c4F)nnc(C)c3cc21 ; COCC1(C)C(=O)N(C)c2cc3c(N[C@H](C)c4cccc(C(F)(F)CO)c4C)nnc(C)c3cc21]

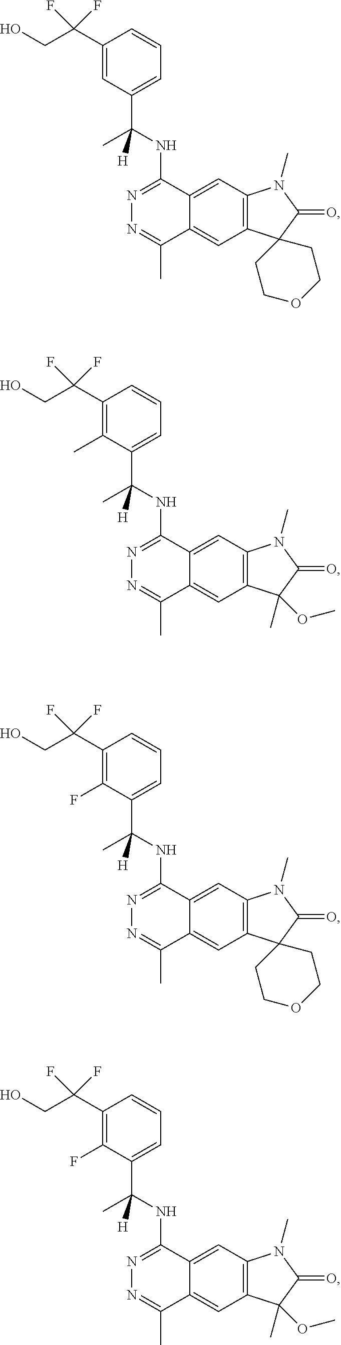 [SMILES: Cc1nnc(N[C@H](C)c2cccc(C(F)(F)CO)c2)c2cc3c(cc12)C1(CCOCC1)C(=O)N3C ; COC1(C)C(=O)N(C)c2cc3c(N[C@H](C)c4cccc(C(F)(F)CO)c4C)nnc(C)c3cc21 ; Cc1nnc(N[C@H](C)c2cccc(C(F)(F)CO)c2F)c2cc3c(cc12)C1(CCOCC1)C(=O)N3C ; COC1(C)C(=O)N(C)c2cc3c(N[C@H](C)c4cccc(C(F)(F)CO)c4F)nnc(C)c3cc21]

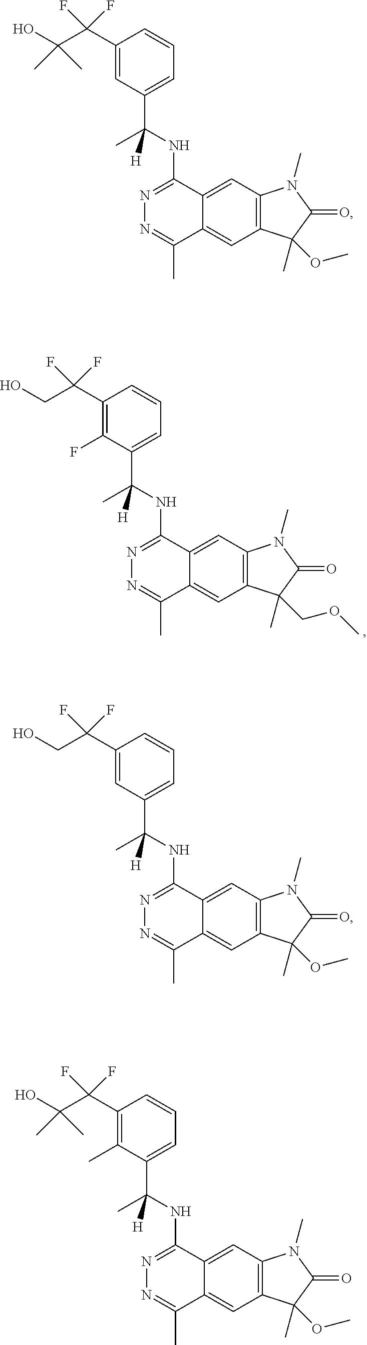 [SMILES: COC1(C)C(=O)N(C)c2cc3c(N[C@H](C)c4cccc(C(F)(F)C(C)(C)O)c4)nnc(C)c3cc21 ; COCC1(C)C(=O)N(C)c2cc3c(N[C@H](C)c4cccc(C(F)(F)CO)c4F)nnc(C)c3cc21 ; COC1(C)C(=O)N(C)c2cc3c(N[C@H](C)c4cccc(C(F)(F)CO)c4)nnc(C)c3cc21 ; COC1(C)C(=O)N(C)c2cc3c(N[C@H](C)c4cccc(C(F)(F)C(C)(C)O)c4C)nnc(C)c3cc21]

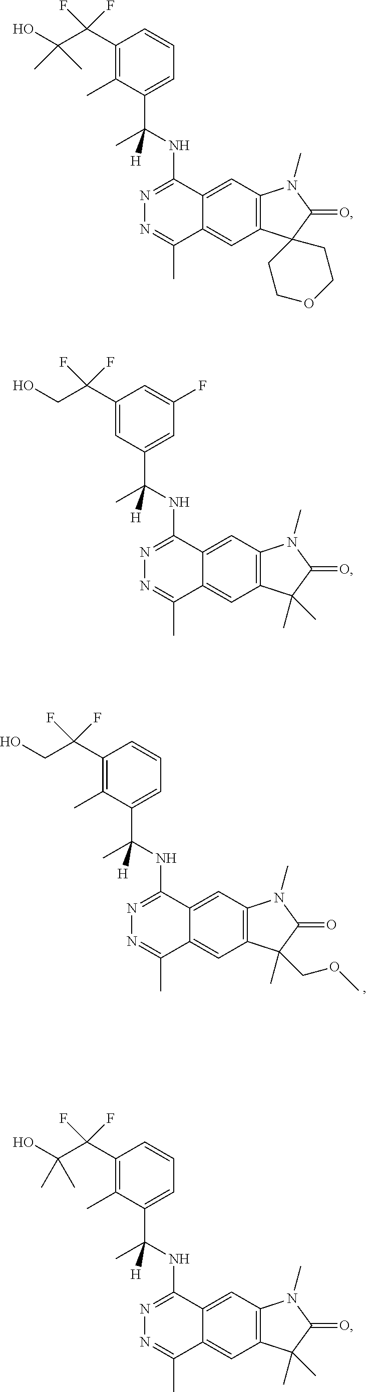 [SMILES: Cc1c([C@@H](C)Nc2nnc(C)c3cc4c(cc23)N(C)C(=O)C42CCOCC2)cccc1C(F)(F)C(C)(C)O ; Cc1nnc(N[C@H](C)c2cc(F)cc(C(F)(F)CO)c2)c2cc3c(cc12)C(C)(C)C(=O)N3C ; COCC1(C)C(=O)N(C)c2cc3c(N[C@H](C)c4cccc(C(F)(F)CO)c4C)nnc(C)c3cc21 ; Cc1c([C@@H](C)Nc2nnc(C)c3cc4c(cc23)N(C)C(=O)C4(C)C)cccc1C(F)(F)C(C)(C)O]

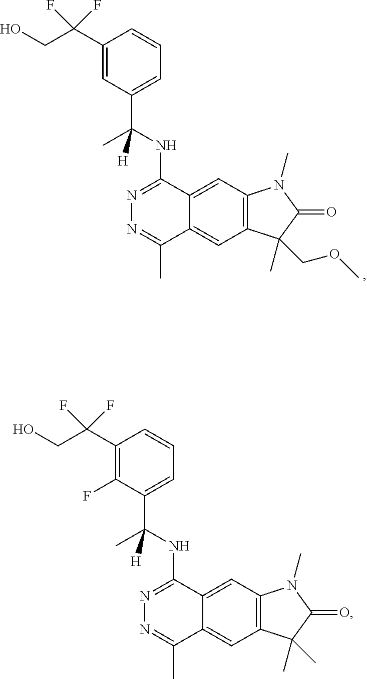 [SMILES: COCC1(C)C(=O)N(C)c2cc3c(N[C@H](C)c4cccc(C(F)(F)CO)c4)nnc(C)c3cc21 ; Cc1nnc(N[C@H](C)c2cccc(C(F)(F)CO)c2F)c2cc3c(cc12)C(C)(C)C(=O)N3C]

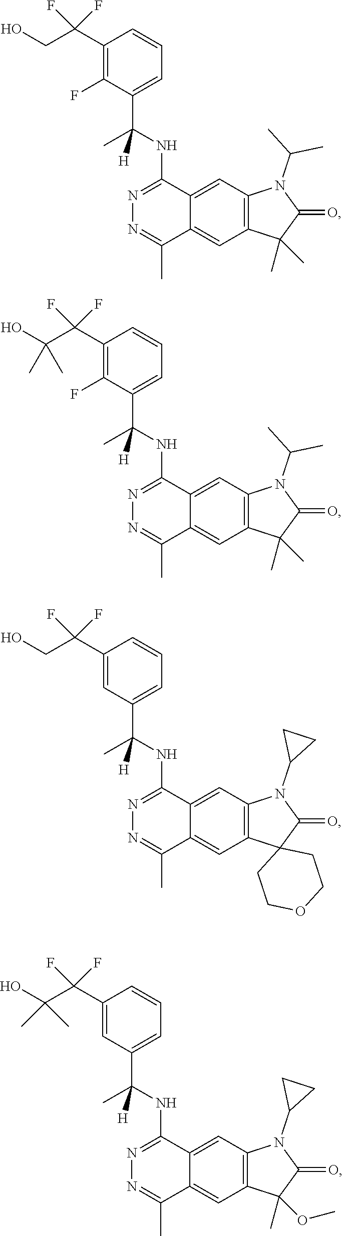 [SMILES: Cc1nnc(N[C@H](C)c2cccc(C(F)(F)CO)c2F)c2cc3c(cc12)C(C)(C)C(=O)N3C(C)C ; Cc1nnc(N[C@H](C)c2cccc(C(F)(F)C(C)(C)O)c2F)c2cc3c(cc12)C(C)(C)C(=O)N3C(C)C ; Cc1nnc(N[C@H](C)c2cccc(C(F)(F)CO)c2)c2cc3c(cc12)C1(CCOCC1)C(=O)N3C1CC1 ; COC1(C)C(=O)N(C2CC2)c2cc3c(N[C@H](C)c4cccc(C(F)(F)C(C)(C)O)c4)nnc(C)c3cc21]

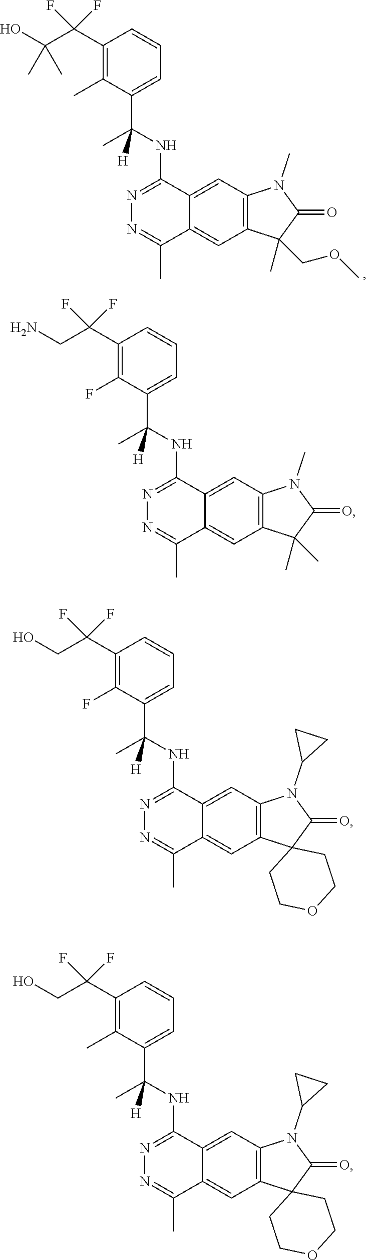 [SMILES: COCC1(C)C(=O)N(C)c2cc3c(N[C@H](C)c4cccc(C(F)(F)C(C)(C)O)c4C)nnc(C)c3cc21 ; Cc1nnc(N[C@H](C)c2cccc(C(F)(F)CN)c2F)c2cc3c(cc12)C(C)(C)C(=O)N3C ; Cc1nnc(N[C@H](C)c2cccc(C(F)(F)CO)c2F)c2cc3c(cc12)C1(CCOCC1)C(=O)N3C1CC1 ; Cc1c([C@@H](C)Nc2nnc(C)c3cc4c(cc23)N(C2CC2)C(=O)C42CCOCC2)cccc1C(F)(F)CO]

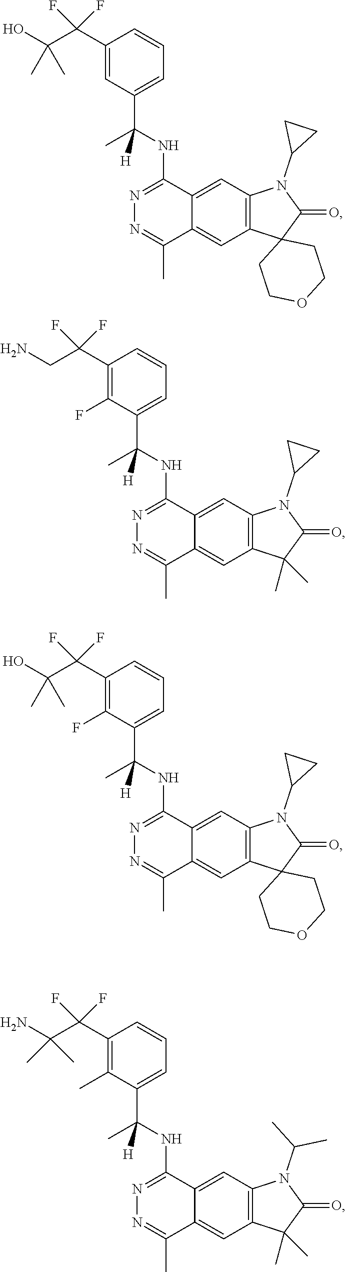 [SMILES: Cc1nnc(N[C@H](C)c2cccc(C(F)(F)C(C)(C)O)c2)c2cc3c(cc12)C1(CCOCC1)C(=O)N3C1CC1 ; Cc1nnc(N[C@H](C)c2cccc(C(F)(F)CN)c2F)c2cc3c(cc12)C(C)(C)C(=O)N3C1CC1 ; Cc1nnc(N[C@H](C)c2cccc(C(F)(F)C(C)(C)O)c2F)c2cc3c(cc12)C1(CCOCC1)C(=O)N3C1CC1 ; Cc1c([C@@H](C)Nc2nnc(C)c3cc4c(cc23)N(C(C)C)C(=O)C4(C)C)cccc1C(F)(F)C(C)(C)N]

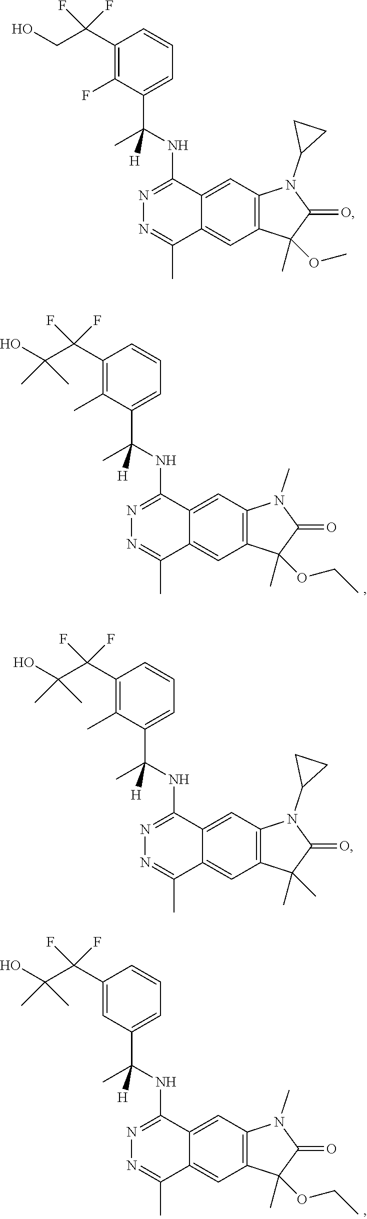 [SMILES: COC1(C)C(=O)N(C2CC2)c2cc3c(N[C@H](C)c4cccc(C(F)(F)CO)c4F)nnc(C)c3cc21 ; CCOC1(C)C(=O)N(C)c2cc3c(N[C@H](C)c4cccc(C(F)(F)C(C)(C)O)c4C)nnc(C)c3cc21 ; Cc1c([C@@H](C)Nc2nnc(C)c3cc4c(cc23)N(C2CC2)C(=O)C4(C)C)cccc1C(F)(F)C(C)(C)O ; CCOC1(C)C(=O)N(C)c2cc3c(N[C@H](C)c4cccc(C(F)(F)C(C)(C)O)c4)nnc(C)c3cc21]

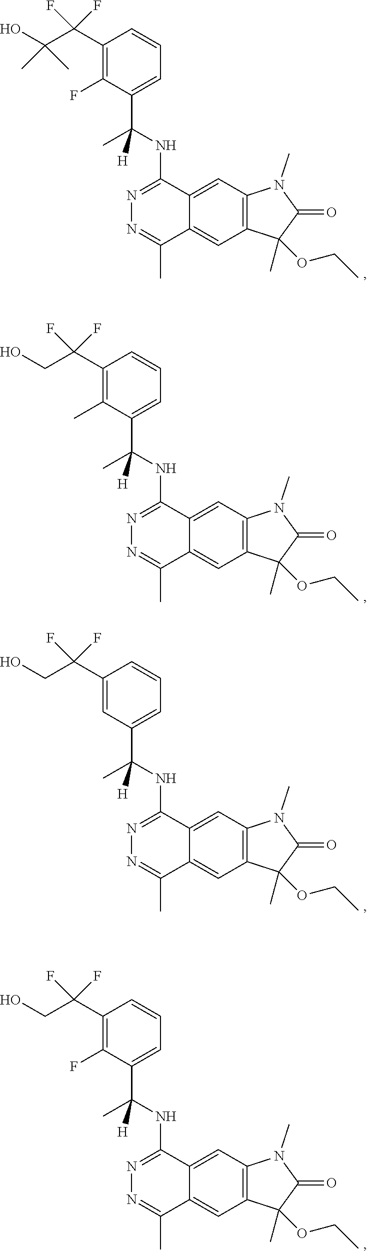 [SMILES: CCOC1(C)C(=O)N(C)c2cc3c(N[C@H](C)c4cccc(C(F)(F)C(C)(C)O)c4F)nnc(C)c3cc21 ; CCOC1(C)C(=O)N(C)c2cc3c(N[C@H](C)c4cccc(C(F)(F)CO)c4C)nnc(C)c3cc21 ; CCOC1(C)C(=O)N(C)c2cc3c(N[C@H](C)c4cccc(C(F)(F)CO)c4)nnc(C)c3cc21 ; CCOC1(C)C(=O)N(C)c2cc3c(N[C@H](C)c4cccc(C(F)(F)CO)c4F)nnc(C)c3cc21]

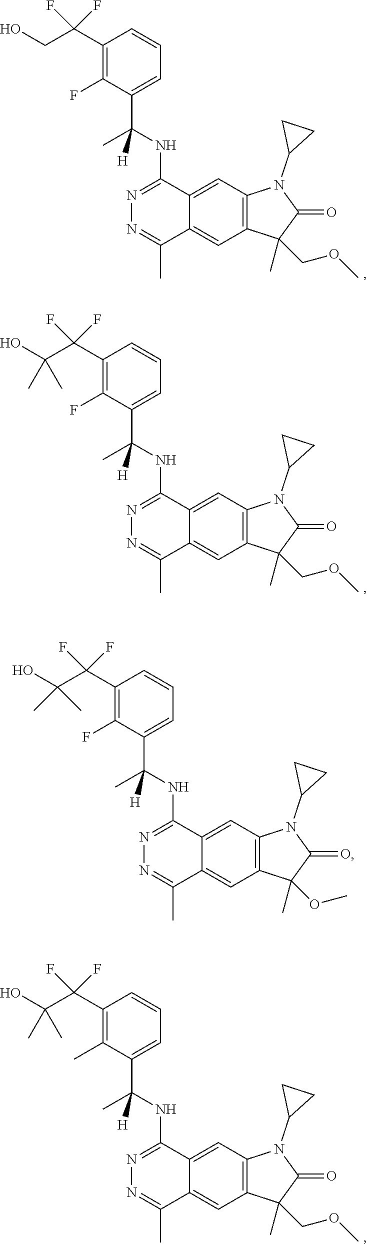 [SMILES: COCC1(C)C(=O)N(C2CC2)c2cc3c(N[C@H](C)c4cccc(C(F)(F)CO)c4F)nnc(C)c3cc21 ; COCC1(C)C(=O)N(C2CC2)c2cc3c(N[C@H](C)c4cccc(C(F)(F)C(C)(C)O)c4F)nnc(C)c3cc21 ; COC1(C)C(=O)N(C2CC2)c2cc3c(N[C@H](C)c4cccc(C(F)(F)C(C)(C)O)c4F)nnc(C)c3cc21 ; COCC1(C)C(=O)N(C2CC2)c2cc3c(N[C@H](C)c4cccc(C(F)(F)C(C)(C)O)c4C)nnc(C)c3cc21]

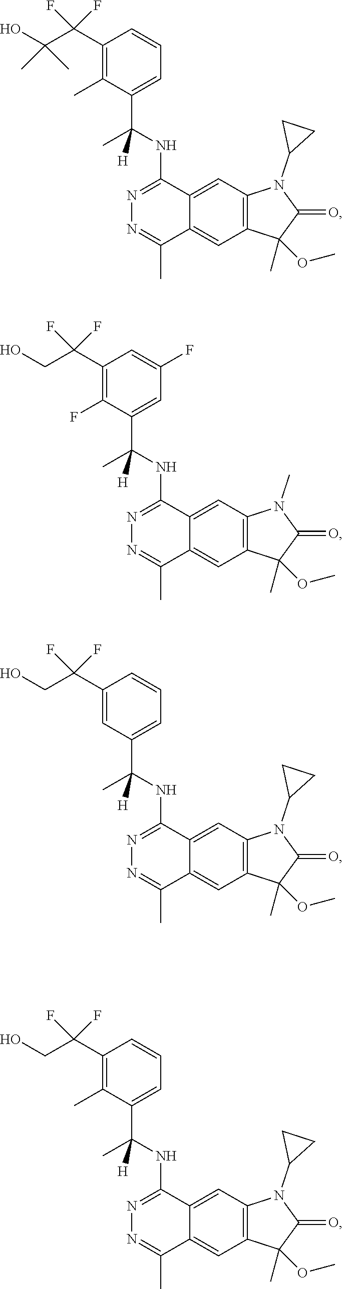 [SMILES: COC1(C)C(=O)N(C2CC2)c2cc3c(N[C@H](C)c4cccc(C(F)(F)C(C)(C)O)c4C)nnc(C)c3cc21 ; COC1(C)C(=O)N(C)c2cc3c(N[C@H](C)c4cc(F)cc(C(F)(F)CO)c4F)nnc(C)c3cc21 ; COC1(C)C(=O)N(C2CC2)c2cc3c(N[C@H](C)c4cccc(C(F)(F)CO)c4)nnc(C)c3cc21 ; COC1(C)C(=O)N(C2CC2)c2cc3c(N[C@H](C)c4cccc(C(F)(F)CO)c4C)nnc(C)c3cc21]

or pharmaceutically acceptable salt thereof.

In some embodiments, the compound of the present disclosure is a compound provided in Table 4A, 4B, 4C, or 4D), or a pharmaceutically acceptable salt thereof.

In some embodiments, the compound of the present disclosure is a compound provided in Table 5 or a pharmaceutically acceptable salt thereof.

In some embodiments, the present disclosure provides a pharmaceutical composition comprising a compound disclosed herein or pharmaceutically acceptable salt thereof, and a pharmaceutically acceptable excipient.

In some embodiments, the present disclosure provides a method of treating and/or preventing cancer comprising administering to a subject a therapeutically effective amount of a compound disclosed herein (e.g., a compound of Formula (I), Formula (Ia), Formula (Ib), and Formula (Ic)), a pharmaceutically acceptable salt thereof, or a pharmaceutical composition thereof.

In some embodiments, the present disclosure provides a method of treating and/or preventing a disease by inhibiting the interaction of SOS1 and a RAS-family protein or RAC1, the method comprising administering to a subject a therapeutically effective amount of a compound disclosed herein (e.g., a compound of Formula (I), Formula (Ia), Formula (Ib), and Formula (Ic)), a pharmaceutically acceptable salt thereof, or a pharmaceutical composition thereof.

### DETAILED DESCRIPTION

### Definitions

While the following terms are believed to be well understood by one of ordinary skill in the art, the following definitions are set forth to facilitate explanation of the presently disclosed subject matter.

The term “a” or “an” refers to one or more of that entity; for example, “a SOS1 inhibitor” refers to one or more SOS1 inhibitors or at least one SOS1 inhibitor. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl.

Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, and the like. The alkylene chain is attached to the rest of the molecule through a single bond and to a radical group (e.g., those described herein) through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to an unsaturated, straight or branched divalent hydrocarbon chain radical having one or more olefins and from two to twelve carbon atoms. Non-limiting examples of C₂-C₁₂ alkenylene include ethenylene, propenylene, n-butenylene, and the like. The alkenylene chain is attached to the rest of the molecule through a single bond and to a radical group (e.g., those described herein) through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂alkynyl includes all the foregoing moieties, but also includes C₁₁ and C₁₂ alkynyls. Non-limiting examples of C₂-C₁₂ alkenyl include ethynyl, propynyl, butynyl, pentynyl and the like.

Unless stated otherwise specifically in the specification, an alkyl group can be optionally substituted.

“Alkynylene” or “alkynylene chain” refers to an unsaturated, straight or branched divalent hydrocarbon chain radical having one or more alkynes and from two to twelve carbon atoms. Non-limiting examples of C₂-C₁₂ alkynylene include ethynylene, propynylene, n-butynylene, and the like. The alkynylene chain is attached to the rest of the molecule through a single bond and to a radical group (e.g., those described herein) through a single bond. The points of attachment of the alkynylene chain to the rest of the molecule and to the radical group can be through any two carbons within the chain having a suitable valency. Unless stated otherwise specifically in the specification, an alkynylene chain can be optionally substituted.

“Alkoxy” refers to a group of the formula —ORₐ where Rₐ is an alkyl, alkenyl or alknyl as defined above containing one to twelve carbon atoms. Unless stated otherwise specifically in the specification, an alkoxy group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

“Carbocyclyl,” “carbocyclic ring” or “carbocycle” refers to a rings structure, wherein the atoms which form the ring are each carbon, and which is attached to the rest of the molecule by a single bond. Carbocyclic rings can comprise from 3 to 20 carbon atoms in the ring. Carbocyclic rings include aryls and cycloalkyl, cycloalkenyl, and cycloalkynyl as defined herein. Unless stated otherwise specifically in the specification, a carbocyclyl group can be optionally substituted.

“Carbocyclylalkyl” refers to a radical of the formula —R_{b}-R_{d} where Re is an alkylene, alkenylene, or alkynylene group as defined above and R_{d} is a carbocyclyl radical as defined above.

Unless stated otherwise specifically in the specification, a carbocyclylalkyl group can be optionally substituted.

“Cycloalkyl” refers to a stable non-aromatic monocyclic or polycyclic fully saturated hydrocarbon consisting solely of carbon and hydrogen atoms, which can include fused or bridged ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyls include, for example, cyclopentenyl, cyclohexenyl, cycloheptenyl, cycloctenyl, and the like. Polycyclic cycloalkenyls include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl include, for example, cycloheptynyl, cyclooctynyl, and the like. Unless otherwise stated specifically in the specification, a cycloalkynyl group can be optionally substituted.

“Haloalkyl” refers to an alkyl, as defined above, that is substituted by one or more halo radicals, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Heterocyclyl,” “heterocyclic ring” or “heterocycle” refers to a stable saturated or unsaturated 3- to 20-membered ring which consists of two to nineteen carbon atoms and from one to six heteroatoms selected from the group consisting of nitrogen, oxygen and sulfur, and which is attached to the rest of the molecule by a single bond. Unless stated otherwise specifically in the specification, the heterocyclyl can be a monocyclic, bicyclic, tricyclic or tetracyclic ring system, which can include fused or bridged ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heteroaryl” refers to a 5- to 20-membered ring system comprising hydrogen atoms, one to nineteen carbon atoms, one to six heteroatoms selected from the group consisting of nitrogen, oxygen and sulfur, at least one aromatic ring, and which is attached to the rest of the molecule by a single bond. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

The term “substituted” used herein means any of the groups described herein (e.g., alkyl, alkenyl, alkynyl, alkoxy, aryl, aralkyl, carbocyclyl, cycloalkyl, cycloalkenyl, cycloalkynyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heteroaryl, N-heteroaryl and/or heteroarylalkyl group. In addition, each of the foregoing substituents can also be optionally substituted with one or more of the above substituents.

As used herein, the symbol

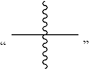

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

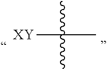

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

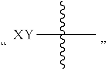

infers that when R³ is “XY”, the point of attachment bond is the same bond as the bond by which R³ is depicted as being bonded to CH₃.

The terms “administer,” “administering” or “administration” as used herein refer to administering a compound or pharmaceutically acceptable salt of the compound or a composition or formulation comprising the compound or pharmaceutically acceptable salt of the compound to a patient.

The term “treating” as used herein with regard to a patient, refers to improving at least one symptom of the patient's disorder. In some embodiments, treating can be improving, or at least partially ameliorating a disorder or one or more symptoms of a disorder.

The term “therapeutically effective” applied to dose or amount refers to that quantity of a compound or pharmaceutical formulation that is sufficient to result in a desired clinical benefit after administration to a patient in need thereof.

### Compounds of the Disclosure

In some embodiments, the present disclosure provides a compound of Formula (A):

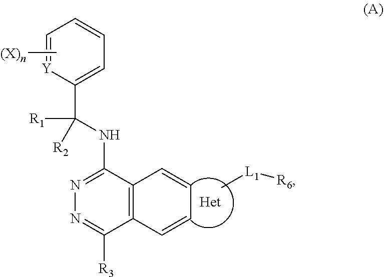

or a pharmaceutically acceptable salt thereof,
wherein:
X is independently halogen, alkyl, alkoxy, amino, amido, nitrile, acyl, cycloalkyl, heterocyclyl, or heteroaryl, and n is an integer from 1-5, and/or two X groups together with the atoms to which they are attached form a heterocyclyl or heteroaryl ring; Y is CH or N;
R₁ and R₂ are each independently hydrogen, alkyl, or R₁ and R₂ together with the atom to which they are attached form a cycloalkyl or heterocyclyl, wherein at least one of R₁ and R₂ is not hydrogen; and
R₃ is hydrogen, alkyl, —(C═O)—OR_{A}, —(C═O)—N(R_{A})₂, cycloalkyl, heterocyclyl, aryl or heteroaryl, wherein each R_{A} is independently hydrogen and alkyl

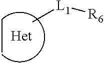

is a nitrogen-containing heterocyclyl substituted with L₁-R₆ and 0-6 substituents independently selected from R₈, R₉, R₁₀, and R₁₁;
L₁ is absent, alkylene, alkenylene, or alkynylene;
R₆ is alkyl, —O-alkyl, cycloalkyl, or heterocyclyl;
R₈ and R₉ are each independently H, halogen, or alkyl, or an R₈ and R₉ together with the carbon atom to which they are attached form a C₃₋₆cycloalkyl, a 3- to 6-membered heterocyclyl, or a carbonyl;
R₁₀ is H, halogen, or -L₂-R₇, wherein L₂ is absent, alkylene, alkenylene, or alkynylene; and
R₇ is H, alkyl, —O-alkyl, cycloalkyl, or heterocyclyl; and
R₁₁ is H, halogen, or alkyl, or an R₁₀ and R₁₁ together with the carbon atom to which they are attached form a C₃₋₆cycloalkyl, a 3- to 6-membered heterocyclyl, or a carbonyl.

In some embodiments, the present disclosure provides a compound of Formula (I):

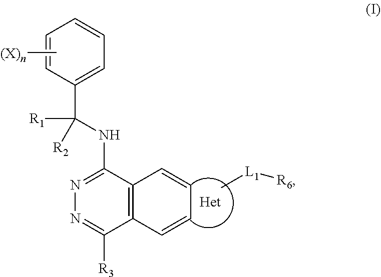

or a pharmaceutically acceptable salt thereof,
wherein:
X is independently halogen, alkyl, alkoxy, amino, amido, nitrile, acyl, cycloalkyl, heterocyclyl, or heteroaryl, and n is an integer from 1-5, and/or two X groups together with the atoms to which they are attached form a heterocyclyl or heteroaryl ring;
R₁ and R₂ are each independently hydrogen, alkyl, or R₁ and R₂ together with the atom to which they are attached form a cycloalkyl or heterocyclyl, wherein at least one of R₁ and R₂ is not hydrogen; and
R₃ is hydrogen, alkyl, —(C═O)—OR_{A}, —(C═O)—N(R_{A})₂, cycloalkyl, heterocyclyl, aryl or heteroaryl, wherein each R_{A} is independently hydrogen or alkyl

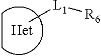

is a nitrogen-containing heterocyclyl substituted with L₁-R₆ and 0-6 substituents independently selected from R₈, R₉, R₁₀, and R₁₁, wherein:
L₁ is absent, alkylene, alkenylene, or alkynylene;
R₆ is alkyl, —O-alkyl, cycloalkyl, or heterocyclyl;
R₈ and R₉ are each independently H, halogen, or alkyl, or an R₈ and R₉ together with the carbon atom to which they are attached form a C₃₋₆cycloalkyl, a 3- to 6-membered heterocyclyl, or a carbonyl;
R₁₀ is H, halogen, or -L₂-R₇, wherein L₂ is absent, alkylene, alkenylene, or alkynylene; and
R₇ is H, alkyl, —O-alkyl, cycloalkyl, or heterocyclyl; and
R₁₁ is H, halogen, or alkyl, or an R₁₀ and R₁₁ together with the carbon atom to which they are attached form a C₃₋₆cycloalkyl, a 3- to 6-membered heterocyclyl, or a carbonyl.

In some embodiments, the present disclosure provides a compound of Formula (I):

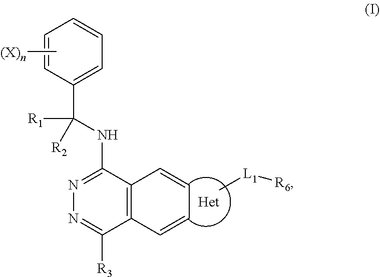

or a pharmaceutically acceptable salt thereof,
wherein:
X is independently halogen, alkyl, alkoxy, amino, amido, nitrile, or acyl, and n is an integer from 1-5, and/or two X groups together with the atoms to which they are attached form a heterocyclyl or heteroaryl ring;
R₁ and R₂ are each independently hydrogen, alkyl, or R₁ and R₂ together with the atom to which they are attached form a cycloalkyl or heterocyclyl, wherein at least one of R₁ and R₂ is not hydrogen; and
R₃ is hydrogen, alkyl, —(C═O)—OR_{A}, —(C═O)—N(R_{A})₂, cycloalkyl, heterocyclyl, aryl or heteroaryl, wherein each R_{A} is independently hydrogen or alkyl

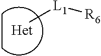

is a nitrogen-containing heterocyclyl substituted with L₁-R₆ and 0-6 substituents independently selected from R₈, R₉, R₁₀, and R₁₁, wherein:
L₁ is absent, alkylene, alkenylene, or alkynylene;
R₆ is alkyl, —O-alkyl, cycloalkyl, or heterocyclyl;
R₈ and R₉ are each independently H, halogen, or alkyl, or an R₈ and R₉ together with the carbon atom to which they are attached form a C₃₋₆cycloalkyl, a 3- to 6-membered heterocyclyl, or a carbonyl R₁₀ is H, halogen, or -L₂-R₇, wherein L₂ is absent, alkylene, alkenylene, or alkynylene; and
R₇ is H, alkyl, —O-alkyl, cycloalkyl, or heterocyclyl; and
R₁₁ is H, halogen, or alkyl, or an R₁₀ and R₁₁ together with the carbon atom to which they are attached form a C₃₋₆cycloalkyl, a 3- to 6-membered heterocyclyl, or a carbonyl.

In some embodiments of Formula (I),

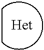

is a 5- to 14-membered nitrogen-containing heterocyclyl. In some embodiments,

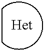

is a 5- to 14-membered nitrogen-containing heterocyclyl having 1, 2, or 3 heteroatoms selected from the group consisting of N, O, and S. In some embodiments,

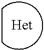

is a 5- to 14-membered heterocyclyl having 1-3 heteroatoms selected from nitrogen and oxygen, wherein at least one of the heteroatoms is nitrogen. In some embodiments,

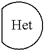

is a 5- or 6-membered nitrogen-containing heterocyclyl having 1, 2, or 3 heteroatoms selected from the group consisting of N, O, and S. In some embodiments,

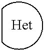

is a 5- or 6-membered heterocyclyl having 1-3 heteroatoms selected from nitrogen and oxygen, wherein at least one of the heteroatoms is nitrogen. In some embodiments,

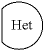

is a 5-membered heterocyclyl having 1-3 heteroatoms selected from nitrogen and oxygen, wherein at least one of the heteroatoms is nitrogen. In some embodiments,

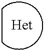

is a 6-membered heterocyclyl having 1-3 heteroatoms selected from nitrogen and oxygen, wherein at least one of the heteroatoms is nitrogen. In some embodiments,

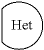

is a heterocyclyl comprising 1-3 nitrogen atoms. In some embodiments,

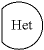

is selected from the group consisting of pyrazole, pyridine, pyrimidine, pyrazine, pyridazine, pyrimidone, pyridone, or derivative thereof.

In some embodiments,

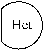

is optionally substituted with alkyl, alkoxy, halogen, oxo, —(C═O)—OR_{A}, —(C═O)—N(R_{A})₂, cycloalkyl, heterocyclyl, aryl or heteroaryl. In some embodiments,

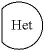

is optionally substituted with alkyl, halogen, oxo, —(C═O)—OR_{A}, —(C═O)—N(R_{A})₂, cycloalkyl, heterocyclyl, aryl or heteroaryl, wherein each R_{A} is independently hydrogen or alkyl. In some embodiments,

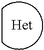

is optionally substituted with oxo, alkyl, or halogen. In some embodiments,

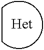

is optionally substituted with alkyl or halogen. In some embodiments,

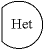

is optionally substituted with alkyl, e.g., methyl, ethyl, n-propyl, isopropyl, n-butyl, isobutyl, t-butyl, isoamyl, or neopentyl. In some embodiments,

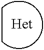

is optionally substituted with one or more halogen. In some embodiments, the halogen is F, Br, or Cl. In some embodiments,

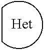

is optionally substituted with alkyl, alkoxy, halogen, —(C═O)—OR_{A}, or —(C═O)—N(R_{A})₂, wherein each R_{A} is independently hydrogen or alkyl. In some embodiments,

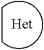

is substituted with a C₁₋₅alkyl. In some embodiments,

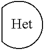

is substituted with methyl, ethyl, n-propyl, isopropyl, n-butyl, t-butyl, isoamyl or neopentyl. In some embodiments,

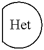

is substituted with methyl. In some embodiments,

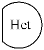

is substituted with C₁₋₅alkoxy, e.g., methoxy, ethoxy, n-propoxy, isopropoxy, n-butoxy, t-butoxy, and the like.

In some embodiments,

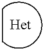

is substituted with C₃₋₈ cycloalkyl. In some embodiments,

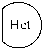

is substituted with cyclopropyl. In some embodiments,

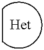

is substituted with a 4- to 12-member heterocyclyl with 1 or 2 heteroatoms selected from N, O, and S. In some embodiments,

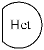

is substituted with a 5- or 6-membered heterocyclyl comprising a heteroatom selected from N, O, and S. In some embodiments,

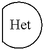

is substituted with a phenyl. In some embodiments,

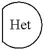

is substituted with a 5- to 14-membered heteroaryl having 1, 2, or 3 heteroatoms selected from N, O, and S. In some embodiments,

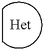

is substituted with a 5- or 6-membered heteroaryl having 1, 2, or 3 heteroatoms selected from N, O, and S. In some embodiments, each R_{A} is independently hydrogen or alkyl. In some embodiments, R_{A} is hydrogen or a C₁₋₅alkyl. In some embodiments, each R_{A} is independently methyl, ethyl, or isopropyl.

In some embodiments,

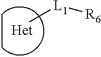

is:

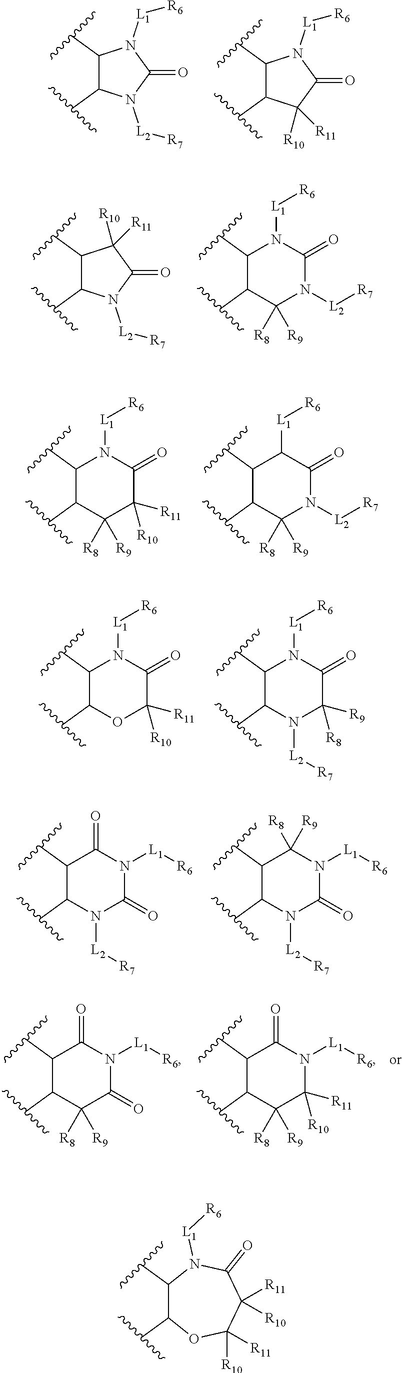

wherein R₆, R₇, R₈, R₉, R₁₀, R₁₁, L₁ and L₂ are as defined in Formula (I). In some embodiments, R₈ and R₉ are each independently H, F, or C₁₋₅alkyl, or R₈ and R₉ together with the carbon atom to which they are attached form a C₃₋₆cycloalkyl or a 3- to 6-membered heterocyclyl. In some embodiments, R₁₀ and R₁₁ are each independently H, F, C₁₋₅alkyl, or -L₂-R₇ and R₁₁ is H, F, or C₁₋₅alkyl, or R₁₀ and R₁₁ together with the carbon atom to which they are attached form a C₃₋₆cycloalkyl or a 3- to 6-membered heterocyclyl. In some embodiments, R₁₀ and R₁₁ are each C₁₋₅alkyl. In some embodiments, R₁₀ and R₁₁ are each CH₃.

In some embodiments,

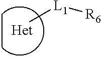

is selected from the group consisting of:

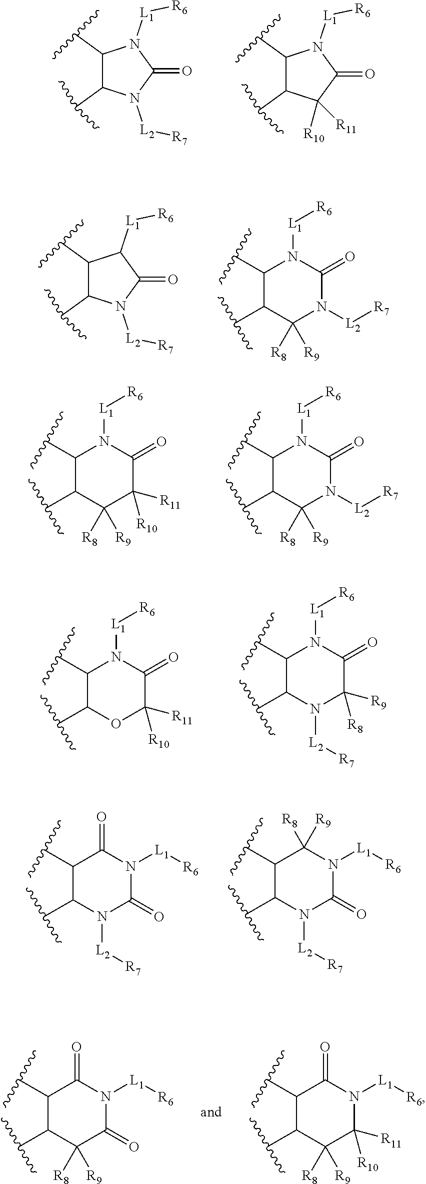

wherein R₆, R₇, R₈, R₉, R₁₀, R₁₁, L₁ and L₂ are as defined in Formula (I). In some embodiments, R₈ and R₉ are each independently H, F, or C₁₋₅alkyl, or R₈ and R₉ together with the carbon atom to which they are attached form a C₃₋₆cycloalkyl or a 3- to 6-membered heterocyclyl. In some embodiments, R₁₀ is H, F, C₁₋₅alkyl, or -L₂-R₇ and R₁₁ is H, F, or C₁₋₅alkyl, or R₁₀ and R₁₁ together with the carbon atom to which they are attached form a C₃₋₆cycloalkyl or a 3- to 6-membered heterocyclyl. In some embodiments, R₁₀ and R₁₁ are each C₁₋₅alkyl. In some embodiments, R₁₀ and R₁ are each CH₃.

In some embodiments,

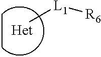

is:

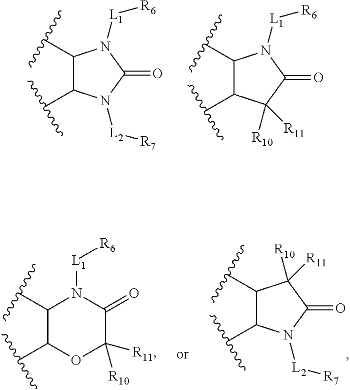

wherein R₆, R₇, R₁₀, R₁, L₁ and L₂ are as defined herein.

In some embodiments,

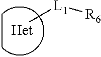

is:

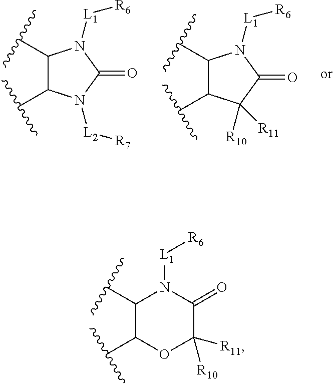

wherein R₆, R₇, R₁₀, Ru, L₁ and L₂ are as defined herein. In some embodiments, R₁₀ and R₁₁ are each C₁₋₅alkyl. In some embodiments, R₁₀ and R₁₁ are each CH₃.

In some embodiments, each X is independently halogen, alkyl, alkoxy or amino. In some embodiments, each X is independently halogen, haloalkyl, haloalkoxy, or amino. In some embodiments, each X is independently halogen, haloalkyl or amino. In some embodiments, each X is independently halogen, alkyl, or amino. In some embodiments, the alkyl is substituted with one or more halogen, hydroxyl, alkoxy, amino, or combination thereof. In some embodiments, each X is independently halogen, haloalkyl or amino. In some embodiments, the haloalkyl is a fluoroalkyl. In some embodiments, each X is independently halogen, alkyl, —NH₂, or alkoxy. In some embodiments, each X is independently a fluoroalkyl, fluoroalkoxy, F or —NH₂. In some embodiments, the fluoroalkyl is —CF₂CH₂O—C₁₋₅alkyl, —CF₂C(C₁₋₅alkyl)₂O—C₁₋₅alkyl, —CF₂CH₂O—C₃₋₆cycloalkyl, —CF₂C(C₁₋₅alkyl)₂₀-cycloalkyl, —CF₂CH₂OH, or —CF₂C(C₁₋₅alkyl)₂OH. In some embodiments, each C₁₋₅alkyl is independently methyl, ethyl, or isopropyl. In some embodiments, the C₃₋₆cycloalkyl is cyclopropyl. In some embodiments, each X is independently halogen, C₁₋₅alkyl, —NH₂, or C₁₋₅alkoxy. In some embodiments, each X is independently halogen, C₁₋₅alkyl, or —NH₂. In some embodiments, each X is independently a C₁₋₅haloalkyl. In some embodiments, the C₁₋₅alkyl is a C₁₋₅fluoroalkyl. In some embodiments, each X is independently —CH₂F, —CHF₂, —CF₃, —CF₂CH₂OCH₃, —CF₂C(CH₃)₂OCH₃, —CF₂CH₂OcPr, —CF₂C(CH₃)₂OcPr, —CF₂CH₂OH, —CF₂C(CH₃)₂OH, —CF₂C(CH₃)(CH₂OMe)OH, —CF₂C(CH₃)(CH₂NHMe)OH, —CF₂C(CH₃)(CH₂NMe₂)OH, —CF₂CH₂NH₂, —CF₂CH₂NMe₂, —CF₂C(CH₃)₂NH₂, or —CF₂C(CH₃)₂NMe₂. In some embodiments, each X is independently —CH₂F, —CHF₂, —CF₃, —CF₂CH₂OH, —CF₂C(CH₃)₂OH, —CF₂C(CH₃)(CH₂OMe)OH, —CF₂C(CH₃)(CH₂NHMe)OH, —CF₂C(CH₃)(CH₂NMe₂)OH, —CF₂CH₂NH₂, —CF₂CH₂NMe₂, —CF₂C(CH₃)₂NH₂, or —CF₂C(CH₃)₂NMe₂. In some embodiments, each X is independently a C₁₋₅haloalkoxy. In some embodiments, each X is independently —OCF₂CF₃, —OCF₂CH₃, —OCF₂CH₂OH, —OCF₂C(CH₃)₂OH, —OCHF₂, —OCF₃, or —OCH₂F. In some embodiments, each X is independently —OCHF₂, —OCF₃, or —OCH₂F. In some embodiments, each X is independently F, Br, or Cl. In some embodiments, each X is independently F. In some embodiments, each X is independently —CH₂F, —CHF₂, —CF₃, —CF₂CH₂OH, —CF₂C(CH₃)₂OH, —CF₂C(CH₃)(CH₂OMe)OH, —CF₂C(CH₃)(CH₂NHMe)OH, —CF₂C(CH₃)(CH₂NMe₂)OH, —CF₂CH₂NH₂, —CF₂CH₂NMe₂, —CF₂C(CH₃)₂NH₂, —CF₂C(CH₃)₂NMe₂, F, or —NH₂. In some embodiments, each X is independently —CF₂CH₃, —CF₂CH₂OH, —CF₂C(CH₃)₂OH, —CH₂F, —CHF₂, —CF₃, F, or —NH₂. In some embodiments, each X is independently —CH₂F, —CHF₂, —CF₃, —CF₂CH₂OH, —CF₂C(CH₃)₂OH, —CF₂C(CH₃)(CH₂OMe)OH, —CF₂C(CH₃)(CH₂NHMe)OH, —CF₂C(CH₃)(CH₂NMe₂)OH, —CF₂C(CH₃)₂NH₂, F, or —NH₂. In some embodiments, each X is independently —CF₂CH₃, —CF₂CH₂OH, —CF₂C(CH₃)₂OH, —CHF₂, —CF₃, F, or —NH₂. In some embodiments, each X is independently —CH₂F, —CHF₂, —CF₃, F, or —NH₂. In some embodiments, each X is independently —CHF₂, —CF₃, F, or —NH₂.

In some embodiments,

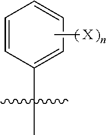

is:

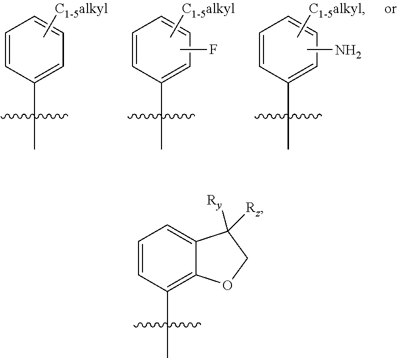

wherein R_{y} and R_{z} are each independently H, F, or alkyl, or an R_{y} and R_{z} together with the carbon atom to which they are attached form a C₃₋₆cycloalkyl. In some embodiments, the C₁₋₅alkyl is a C₁₋₅haloalkyl. In some embodiments, the C₁₋₅alkyl is a C₁₋₅haloalkyl, optionally substituted with —OH or —NH₂. In some embodiments, the C₁₋₅alkyl is a C₁₋₅fluoroalkyl. In some embodiments, the C₁₋₅alkyl is a C₁₋₅fluoroalkyl, optionally substituted with —OH or —NH₂. In some embodiments, the C₁₋₅alkyl is —CH₂F, —CHF₂, —CF₃, —CF₂CH₂OH, —CF₂C(CH₃)₂OH, —CF₂C(CH₃)(CH₂OMe)OH, —CF₂C(CH₃)(CH₂NHMe)OH, —CF₂C(CH₃)(CH₂NMe₂)OH, —CF₂CH₂NH₂, —CF₂CH₂NMe₂, —CF₂C(CH₃)₂NH₂, or —CF₂C(CH₃)₂NMe₂. In some embodiments, the C₁₋₅alkyl is —CH₂F, —CHF₂, —CF₃, —CF₂CH₂OH, —CF₂C(CH₃)₂OH, —CF₂C(CH₃)(CH₂OMe)OH, —CF₂C(CH₃)(CH₂NHMe)OH, —CF₂C(CH₃)(CH₂NMe₂)OH, or —CF₂C(CH₃)₂NH₂. In some embodiments, the C₁₋₅alkyl is —CF₂CH₂OH, —CF₂C(CH₃)₂OH, —CF₂C(CH₃)₂NH₂, —CHF₂, or —CF₃. In some embodiments, the C₁₋₅alkyl is —CF₂CF₃, —CF₂CH₃, —CF₂CH₂OH, —CF₂C(CH₃)₂OH, —CHF₂, —CF₃, or —CH₂F. In some embodiments, R_{y} and R_{z} are each H. In some embodiments, R_{y} and R_{z} are each F. In some embodiments, R_{y} and R_{z} are each Me. In some embodiments, R_{y} and R_{z} together with the carbon atom to which they are attached form a cyclopropyl.

In some embodiments of Formula (I),

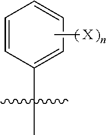

is:

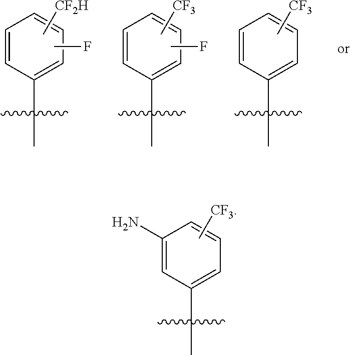

In some embodiments,

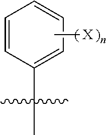

is:

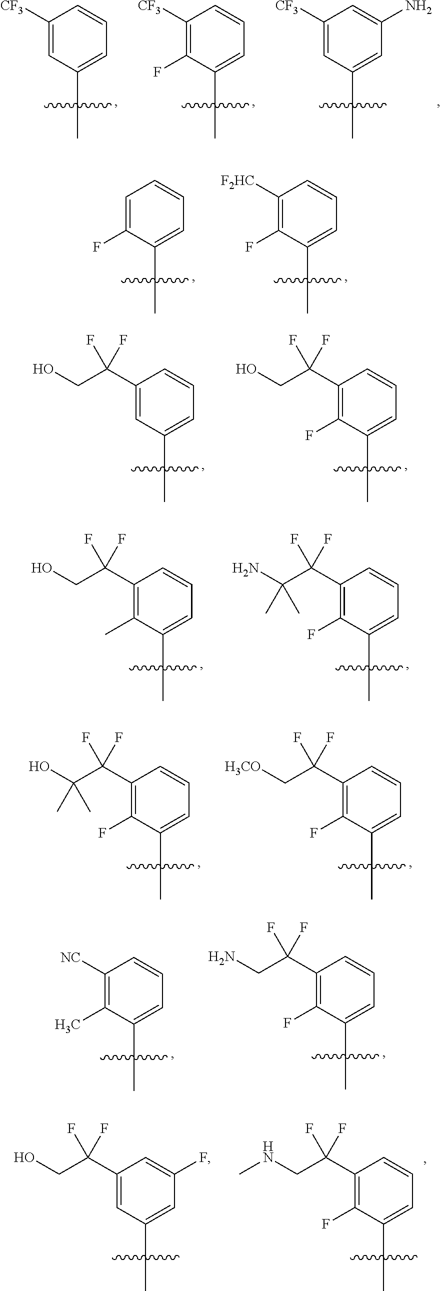

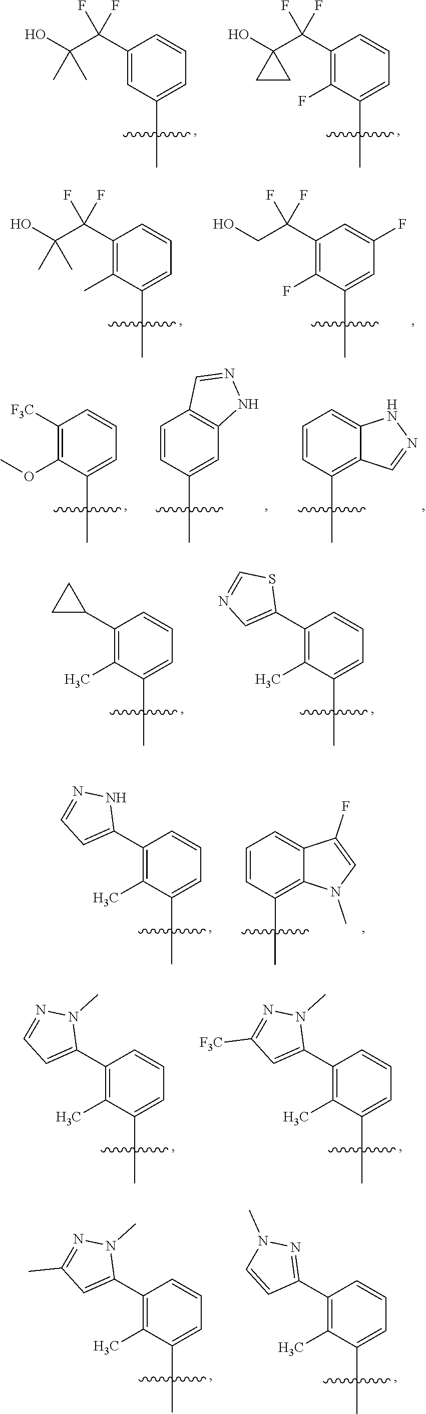

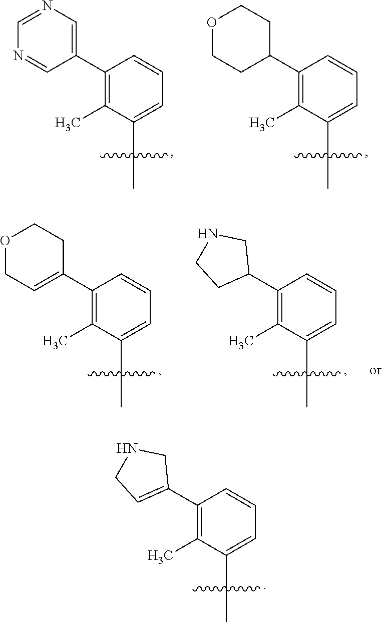 [SMILES: *c1cccc(-c2cncnc2)c1C ; *c1cccc(C2CCOCC2)c1C ; *c1cccc(C2=CCOCC2)c1C ; *c1cccc(C2CCNC2)c1C ; *c1cccc(C2=CCNC2)c1C]

In some embodiments of Formula (I),

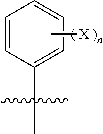

is:

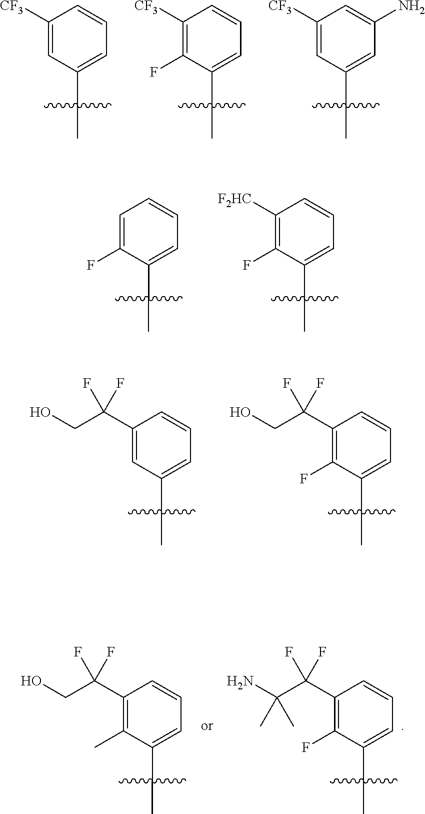

In some embodiments of Formula (I),

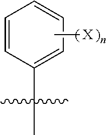

is

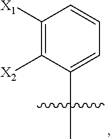

wherein X₁ is C₁₋₅ haloalkyl and X₂ is halogen, C₁₋₃ alkyl, or —O—C₁₋₃ alkyl. In some embodiments, X₁ is CF₃, CHF₂,

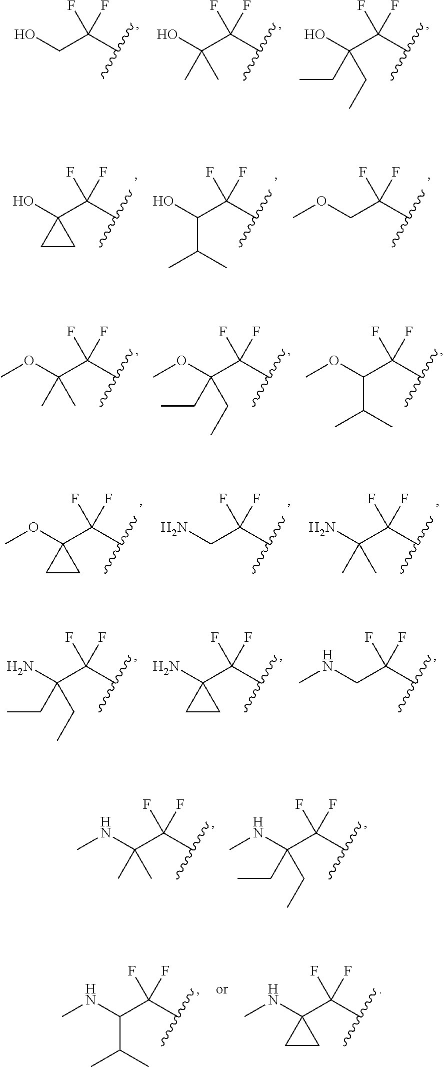

In some embodiments, X₁ is

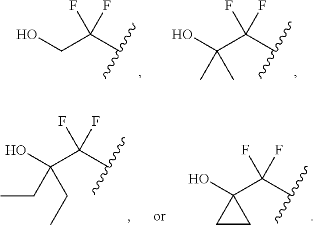

In some embodiments, X₂ is halogen, C₁₋₅alkyl optionally substituted with one or more fluoride, or —OC₁₋₅alkyl optionally substituted with one or more fluoride. In some embodiments, X₂ is F, CH₃ or —OCH₃. In some embodiments, X₂ is F, CH₃ or —OCH₃.

In some embodiments of Formula (I), R₁ and R₂ are independently hydrogen or alkyl (e.g., methyl, ethyl, n-propyl, isopropyl, n-butyl, isobutyl, t-butyl, n-pentyl, isoamyl, neopentyl, and the like), wherein at least one of R₁ and R₂ is not hydrogen. In some embodiments, R₁ and R₂ are independently hydrogen or C₁₋₅ alkyl, wherein at least one of R₁ and R₂ is not hydrogen. In some embodiments, R₁ and R₂ are independently hydrogen or methyl, wherein at least one of R₁ and R₂ is not hydrogen. In some embodiments, R₁ is methyl and R₂ is H. In some embodiments, R₁ and R₂ together with the atom to which they are attached form a cycloalkyl or heterocyclyl. In some embodiments, R₁ and R₂ together with the atom to which they are attached form a cycloalkyl. In some embodiments, R₁ and R₂ together with the atom to which they are attached form a C₃₋₈ cycloalkyl. In some embodiments, R₁ and R₂ together with the atom to which they are attached form a C₃₋₆ cycloalkyl. In some embodiments, R₁ and R₂ together with the atom to which they are attached form a cyclopropyl.

In some embodiments of Formula (I), R₃ is hydrogen, alkyl, —(C═O)—OR_{A}, —(C═O)—N(R_{A})₂, cycloalkyl, heterocyclyl, aryl or heteroaryl. In some embodiments, each R_{A} is independently hydrogen or alkyl. In some embodiments, R₃ is a C₁₋₅alkyl. In some embodiments, R₃ is methyl, ethyl, or isopropyl. In some embodiments, R₃ is hydrogen, alkyl, —(C═O)—OR_{A}, —(C═O)—N(R_{A})₂, cycloalkyl, aryl or heteroaryl. In some embodiments, R₃ is hydrogen, alkyl, —(C═O)—OR_{A}, —(C═O)—N(R_{A})₂, cycloalkyl, or heteroaryl. In some embodiments, R₃ is hydrogen, alkyl, or halogen. In some embodiments, R₃ is hydrogen or alkyl. In some embodiments, R₃ is hydrogen, alkyl, halogen, —(C═O)—OR_{A}, or —(C═O)—N(R_{A})₂. In some embodiments, R₃ is a C₁₋₅alkyl. In some embodiments, R₃ is methyl, ethyl, n-propyl, isopropyl, n-butyl, t-butyl, isoamyl or neopentyl. In some embodiments, R₃ is methyl. In some embodiments, R₃ is C₃₋₈ cycloalkyl. In some embodiments, R₃ is cyclopropyl. In some embodiments, R₃ is a 4- to 12-member heterocyclyl with 1 or 2 heteroatoms selected from N, O, and S. In some embodiments, R₃ is a 5- or 6-membered heterocyclyl comprising a heteroatom selected from N, O, and S. In some embodiments, R₃ is phenyl. In some embodiments, R₃ is a 5- to 14-membered heteroaryl having 1, 2, or 3 heteroatoms selected from N, O, and S. In some embodiments, R₃ is a 5- or 6-membered heteroaryl having 1, 2, or 3 heteroatoms selected from N, O, and S. In some embodiments, R₃ is hydrogen, alkyl, —(C═O)—OCH₃, —(C═O)—OH, or —(C═O)—NH₂. In some embodiments, R₃ is hydrogen or alkyl. In some embodiments, R₃ is C₁₋₅alkyl. In some embodiments, R₃ is methyl, ethyl, isopropyl, n-propyl, —CH₂OH, —CH₂OCH₃, —CH₂N(CH₃)₂, —CH(OH)(CH₃)₂ or —CH₂(OH)CH₃. In some embodiments, R₃ is H or methyl. In some embodiments, R₃ is H. In some embodiments, R₃ is methyl or ethyl. In some embodiments, R₃ is methyl. In some embodiments, R₃ is ethyl.

In some embodiments, L₁ and L₂ are each independently absent, or a linking, wherein the linking group is alkylene, alkylene-O—, alkylene-N(R_{B})—, alkenylene, alkenylene-O—, alkenylene-N(R_{B})—, alkynylene, alkynylene-O—, alkynylene-N(R_{B})—, or cycloalkylene, wherein R_{B} is hydrogen, alkyl, or alkylenecycloalkyl. In some embodiments, R_{B} is hydrogen, alkyl (e.g., C₁_₅ alkyl, C₁₋₃ alkyl, and the like), or alkylenecycloalkyl (e.g., —CH₂cyclopropyl, —CH₂cyclobutyl, —CH₂cyclopentyl, —CH₂cyclohexyl, and the like). In some embodiments, L₁ and L₂ are each independently absent, alkylene, or alkenylene. In some embodiments, L₁ and L₂ are each independently absent or alkylene. In some embodiments, L₁ and L₂ are each alkylene. In some embodiments, L₁ and L₂ are each absent. In some embodiments, L₁ is alkylene and L₂ is absent. In some embodiments, the alkylene is a C₁_alkylene. In some embodiments, the alkylene is a C₁₋₃alkylene. In some embodiments, the alkylene is —CH₂— or —CH₂CH₂—. In some embodiments, the alkylene is —CH₂—. In some embodiments, the alkylene is —CH₂CH₂—. In some embodiments, the alkylene is —CH₂CH₂CH₂—. In some embodiments, the alkylene is substituted with one or more halogens (e.g., F, Cl, and/or Br) and/or one or more alkyl groups (e.g., —CH₃, —CH₂CH₃, —CH₂CH₂CH₃, and the like). In some embodiments, the alkylene is gem-disubstituted. In some embodiments, the alkylene is gem-disubstituted with two halogens as defined herein. In some embodiments, the alkylene is gem-disubstituted with two alkyl groups as defined herein. In some embodiments, two alkyl groups taken together with the atoms to which they are attached form a C₃₋₆cycloalkyl. In some embodiments, two alkyl groups taken together with the atoms to which they are attached form a cyclopropyl. In some embodiments, the alkylene comprises one or more —CF₂, —CHF, —C(H)(CH)₃—, —C(CH₃)₂— and

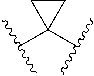 [SMILES: *C1(*)CC1]

groups. In some embodiments, L₁ and L₂ are each independently absent, C₁₋₅alkylene, C₁₋₅alkenylene, or C₁₋₅alkynylene.

In some embodiments of Formula (I), R₆ is alkyl, —O-alkyl, C₃₋₆cycloalkyl, or 3- to 6-membered heterocyclyl. In some embodiments, R₆ is a C₁₋₅alkyl. In some embodiments, the C₁₋₅alkyl is methyl, ethyl, n-propyl, isopropyl, n-butyl, t-butyl, isoamyl or neopentyl. In some embodiments, R₆ is methyl, ethyl, or isopropyl. In some embodiments, R₆ is methyl. In some embodiments, R₆ is a C₃₋₅cycloalkyl. In some embodiments, R₆ is a C₃₋₆cycloalkyl. In some embodiments, R₆ is cyclobutyl, cyclopentyl or cyclohexyl. In some embodiments, R₆ is cyclopentyl. In some embodiments, the cycloalkyl is cyclopentyl. In some embodiments, R₆ is a 4- to 12-membered heterocyclyl. In some embodiments, R₆ is a 4- to 12-membered heterocyclyl with 1 or 2 heteroatoms selected from N, O, and S. In some embodiments, R₆ is a 5- or 6-membered heterocyclyl comprising 1 or 2 oxygen atoms. In some embodiments, R₆ is a 5- or 6-membered heterocyclyl comprising 1 oxygen atom. In some embodiments, R₆ is 3-tetrahydrofuranyl or 3-tetrahydropyranyl. In some embodiments, R₆ is C₁₋₅alkyl, —O—C₁₋₅alkyl, C₃₋₆cycloalkyl, or 3- to 6-membered heterocyclyl. In some embodiments, R₆ is C₁₋₅alkyl. In some embodiments, R₆ is a C₁₋₅haloalkyl. In some embodiments, R₆ is a C₁₋₅fluoroalkyl. In some embodiments, R₆ is methyl, ethyl, isopropyl, tert-butyl, —CH₂CF₃, CH₂CF₂H, —CH(CH₃)CF₃, CH(CH₃)CF₂H, —C(CH₃)₂CF₃, or —C(CH₃)₂CF₂H. In some embodiments, R₆ is methyl, ethyl, isopropyl, or cyclopropyl. In some embodiments, R₆ is methyl, ethyl, n-propyl, or isopropyl. In some embodiments, R₆ is methyl or isopropyl. In some embodiments, R₆ is —O—C₁₋₅alkyl. In some embodiments, R₆ is —OCH₃. In some embodiments, R₆ is an —O—C₁₋₅haloalkyl. In some embodiments, R₆ is an —O—C₁₋₅fluoroalkyl. In some embodiments, R₆ is 3- to 6-membered heterocyclyl or C₃₋₆cycloalkyl. In some embodiments, R₆ is 3-tetrahydrofuranyl or cyclopentyl. In some embodiments, R₆ is 3- to 6-membered heterocyclyl. In some embodiments, R₆ is

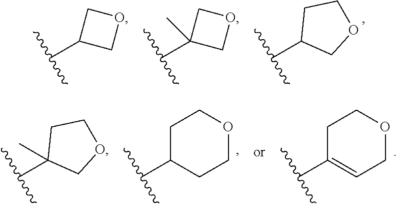

In some embodiments, R₆ is

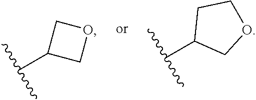 [SMILES: *C1COC1 ; "or" ; *C1CCOC1]

In some embodiments, R₆ is 3-tetrahydrofuranyl

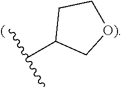

In some embodiments, R₆ is C₃₋₆cycloalkyl. In some embodiments, R₆ is

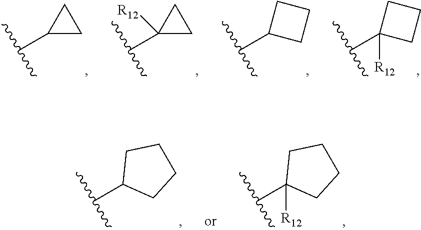

wherein R₁₂ is C₁₋₅alkyl. In some embodiments, R₁₂ is —CH₃, —CF₃ or —CF₂H. In some embodiments, R₆ is

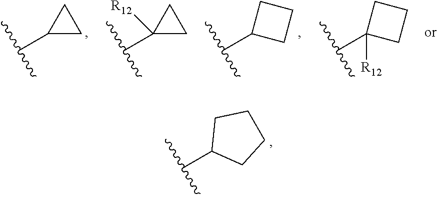

wherein R₁₂ is —CH₃, —CF₃ or —CF₂H. In some embodiments, R₆ is cyclopentyl.

In some embodiments, L₁-R₆ is:

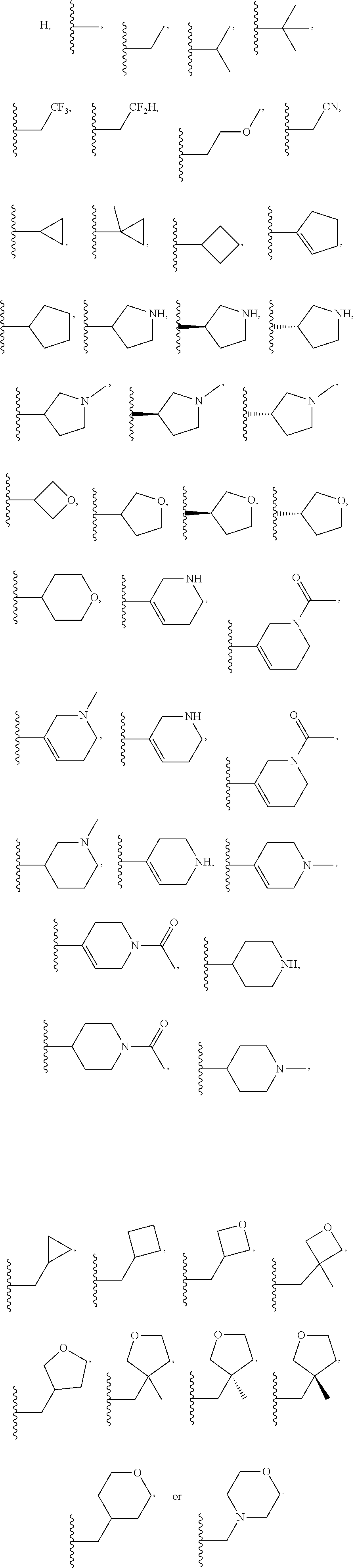

In some embodiments, L₁-R₆ is:

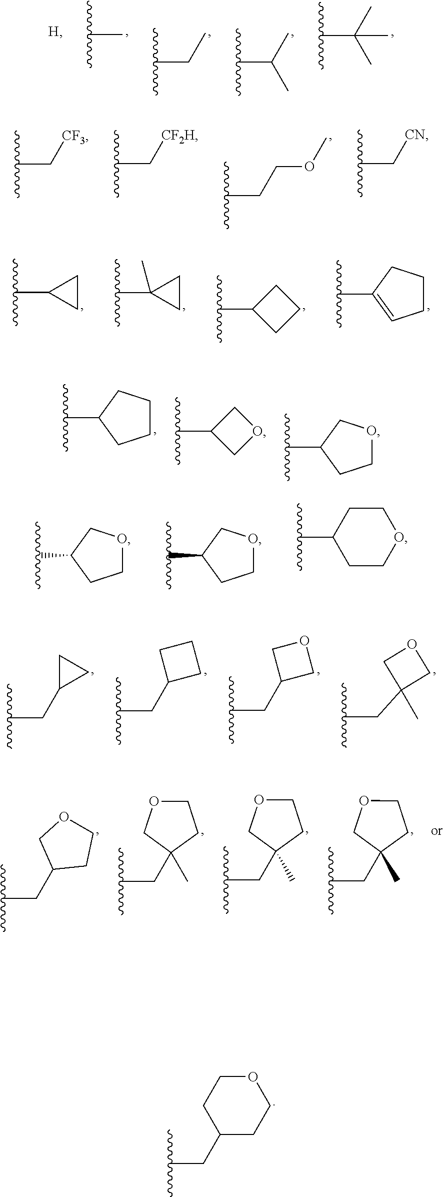

In some embodiments of Formula (I), R₇ is alkyl, —O-alkyl, C₃₋₅cycloalkyl, or 3- to 5-membered heterocyclyl. In some embodiments of Formula (I), R₇ is C₁₋₅alkyl, —O—C₁₋₅alkyl, C₃₋₅cycloalkyl, or 4- to 6-membered heterocyclyl. In some embodiments, R₇ is C₁₋₅alkyl or —O—C₁₋₅alkyl. In some embodiments, R₇ is alkyl. In some embodiments, R₇ is a C₁₋₅alkyl. In some embodiments, R₇ is a C₁₋₅haloalkyl. In some embodiments, R₇ is a C₁₋₅fluoroalkyl. In some embodiments, R₇ is methyl, ethyl, n-propyl, isopropyl, or n-butyl, t-butyl, CF₃ or CHF₂. In some embodiments, R₇ is methyl, ethyl, n-propyl, isopropyl, or n-butyl, t-butyl. In some embodiments, R₇ is methyl. In some embodiments, R₇ is methyl, ethyl, isopropyl, CF₃, CHF₂, —OCH₂CH₃, —OCH₃, cyclopropyl, cyclobutyl, cyclopentyl, cyclohexyl, oxetanyl, or tetrahydrofuranyl. In some embodiments, R₇ is methyl, ethyl, or isopropyl. In some embodiments, the C₁₋₅alkyl is methyl. In some embodiments, R₇ is —O—C₁₋₅alkyl. In some embodiments, R₇ is —OCH₂CH₃ or —OCH₃. In some embodiments, R₇ is —OCH₃. In some embodiments, R₇ is an —O—C₁₋₅haloalkyl. In some embodiments, R₇ is an —O—C₁₋₅fluoroalkyl. In some embodiments, R₇ is C₃₋₅cycloalkyl. In some embodiments, R₇ is cyclopropyl. In some embodiments, R₇ is cyclobutyl. In some embodiments, R₇ is cyclopentyl. In some embodiments, R₇ is 3- to 5-membered heterocyclyl. In some embodiments, R₇ is a 3- to 5-membered heterocyclyl comprising 1 or 2 heteroatoms selected from N, O, and S. In some embodiments, R₇ is a 3- to 5-membered heterocyclyl comprising an oxygen atom. In some embodiments, R₇ is 3-tetrahydrofuranyl.

In some embodiments, R₈ and R₉ are each independently H, halogen, or alkyl, or an R₈ and R₉ together with the carbon atom to which they are attached form a C₃₋₆cycloalkyl or a 3- to 6-membered heterocyclyl. In some embodiments, R₈ and R₉ are each independently H, F, alkyl, alkoxy, or alkylene-cycloalkyl or an R₈ and R₉ together with the carbon atom to which they are attached form a C₃₋₆cycloalkyl or a 3- to 6-membered heterocyclyl. In some embodiments, R₈ and R₉ are each independently H, F, alkyl, or alkylene-cycloalkyl or an R₈ and R₉ together with the carbon atom to which they are attached form a C₃₋₆cycloalkyl or a 3- to 6-membered heterocyclyl. In some embodiments, R₈ and R₉ are each independently H, F, alkyl, or alkylene-cycloalkyl. In some embodiments, R₈ and R₉ are each independently H, F, or alkyl. In some embodiments, R₈ and R₉ are each independently H, F, or C₁₋₅alkyl. In some embodiments, R₈ and R₉ are each H. In some embodiments, R₈ and R₉ are each F. In some embodiments, R₈ and R₉ are each alkyl. In some embodiments, R₈ and R₉ are each methyl. In some embodiments, an R₈ and R₉ together with the carbon atom to which they are attached form a C₃₋₆cycloalkyl. In some embodiments, an R₈ and R₉ together with the carbon atom to which they are attached form a 3- to 6-membered heterocyclyl. In some embodiments, an R₈ and R₉ together with the carbon atom to which they are attached form a 6-membered heterocyclyl.

In some embodiments, R₁₀ is H, F, alkyl, alkoxy, or -L₂-R₇, wherein L₂ and R₇ are as defined herein. In some embodiments, R₁₀ is H, F, alkyl, or -L₂-R₇, wherein L₂ and R₇ are as defined herein. In some embodiments, R₁₀ is H, F, alkyl, or alkoxy. In some embodiments, R₁₀ is H, F, C₁₋₅alkyl, or —O—C₁₋₅alkyl. In some embodiments, R₁₀ is H, F, C₁₋₅alkyl, —CH₂—O—C₁₋₅alkyl, or —O—C₁₋₅alkyl. In some embodiments, R₁₀ is H, F, or alkyl. In some embodiments, R₁₀ is H, F, or C₁₋₅alkyl. In some embodiments, R₁₀ is H. In some embodiments, R₁₀ is F. In some embodiments, R₁₀ is alkyl. In some embodiments, R₁₀ is C₁₋₅alkyl. In some embodiments, R₁₀ is methyl. In some embodiments, R₁₀ is -L₂-R₇, wherein L₂ and R₇ are as defined herein.

In some embodiments, R₁₁ is H, F, alkyl, alkoxy, or alkylene-cycloalkyl. In some embodiments, R₁₁ is H, F, alkyl, or alkoxy. In some embodiments, R₁₁ is H, F, alkyl, or alkylene-cycloalkyl. In some embodiments, R₁₁ is H, F, or alkyl. In some embodiments, R₁₁ is H, F, C₁₋₅alkyl, or —O—C₁₋₅alkyl. In some embodiments, Ru is H, F, C₁₋₅alkyl, —CH₂—O—C₁₋₅alkyl, or —O—C₁₋₅alkyl. In some embodiments, Ru is C₁₋₅alkyl, —CH₂—O—C₁₋₅alkyl, or —O—C₁₋₅alkyl. In some embodiments, Ru is C₁₋₅alkyl or —O—C₁₋₅alkyl. In some embodiments, Ru is H, F, or C₁₋₅alkyl. In some embodiments, Ru is H, F, Me, Et, —OMe, —OEt, or —CH₂—OMe. In some embodiments, Ru is Me, Et, —OMe, —OEt, or —CH₂—OMe. In some embodiments, Ru is H, F, Me, Et, —OMe, or -OEt.

In some embodiments, Ru is Me or —OMe. In some embodiments, Ru is H. In some embodiments, Ru is F. In some embodiments, Ru is alkyl. In some embodiments, Ru is C₁₋₅alkyl. In some embodiments, Ru is methyl.

In some embodiments, R₁₀ is -L₂-R₇ and Ru is H. In some embodiments, R₁₀ is -L₂-R₇ and R₁₁ is F. In some embodiments, R₁₀ is -L₂-R₇ and R₁₁ is C₁₋₅alkyl. In some embodiments, R₁₀ and R₁₁ are each independently H, F, C₁₋₅alkyl, or —O—C₁₋₅alkyl. In some embodiments, R₁₀ and R₁₁ are each independently C₁₋₅alkyl or —O—C₁₋₅alkyl. In some embodiments, R₁₀ and R₁₁ are each independently H, F, or C₁₋₅alkyl. In some embodiments, R₁₀ and R₁₁ are each H. In some embodiments, R₁₀ and R₁₁ are each F. In some embodiments, R₁₀ and R₁₁ are each alkyl. In some embodiments, R₁₀ and R₁₁ are each methyl. In some embodiments, R₁₀ is methyl and Ru is —O—C₁₋₅alkyl. In some embodiments, R₁₀ is methyl and Ru is —OCH₃. In some embodiments, R₁₀ is methyl and Ru is —OCH₂CH₃. In some embodiments, R₁₀ is ethyl and Ru is —OCH₃. In some embodiments, R₁₀ is methyl and Ru is CH₂OCH₃. In some embodiments, an R₁₀ and R₁₁ together with the carbon atom to which they are attached form a carbonyl. In some embodiments, an R₁₀ and Ru together with the carbon atom to which they are attached form a C₃₋₆cycloalkyl. In some embodiments, Ru is H, halogen, or alkyl, or an R₁₀ and R₁₁ together with the carbon atom to which they are attached form a C₃₋₆cycloalkyl or a 3- to 6-membered heterocyclyl. In some embodiments, an R₁₀ and R₁₁ together with the carbon atom to which they are attached form a 3- to 6-membered heterocyclyl. In some embodiments, an R₁₀ and R₁₁ together with the carbon atom to which they are attached form a 6-membered heterocyclyl. In some embodiments, an R₁₀ and R₁₁ together with the carbon atom to which they are attached form

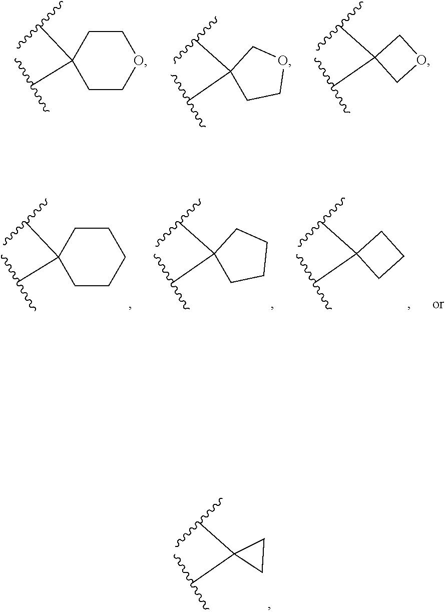

In some embodiments, an R₁₀ and R₁₁ together with the carbon atom to which they are attached form

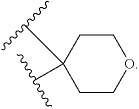 [SMILES: *C1(*)CCOCC1]

In some embodiments of Formula (I),

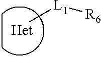

is a 5- or 6-membered heterocyclyl comprising 1-3 heteroatoms selected from nitrogen and oxygen, wherein at least one of the heteroatoms is nitrogen; each X is independently halogen, C₁₋₅alkyl, —NH₂, or C₁₋₅alkoxy; n is an integer from 1-3; R₁ and R₂ are each independently H or C₁₋₅alkyl; L₁ is absent or alkylene; R₃ is hydrogen, alkyl, —(C═O)—OR_{A}, —(C═O)—N(R_{A})₂, or cycloalkyl, wherein R_{A} is as defined herein; R₆ is alkyl, alkoxy, cycloalkyl, or heterocyclyl; R₈ and R₉ are each independently H, F, or alkyl; R₁₀ is H, F, or -L₂-R₇, wherein L₂ is absent or alkylene, and R₇ is H, alkyl, —O-alkyl, cycloalkyl, or heterocyclyl; and R₁₁ is H, F, or alkyl.

In some embodiments of Formula (I),

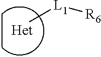

is a 5- or 6-membered heterocyclyl having 1-3 heteroatoms selected from nitrogen and oxygen, wherein at least one of the heteroatoms is nitrogen; each X is independently halogen, C₁₋₅alkyl, —NH₂, or C₁₋₅alkoxy; n is an integer from 1-3; R₁ and R₂ are each independently H or C₁₋₅alkyl; L₁ is absent or alkylene; R₃ is hydrogen, alkyl, or cycloalkyl; R₆ is alkyl, alkoxy, cycloalkyl, or heterocyclyl; R₈ and R₉ are each independently H, F, or alkyl; R₁₀ is H, F, or -L₂-R₇, wherein L₂ is absent or alkylene, and R₇ is H, alkyl, —O-alkyl, cycloalkyl, or heterocyclyl; and Ru is H, F, or alkyl.

In some embodiments of Formula (I),

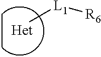

is a 5-membered heterocyclyl having 1-3 heteroatoms selected from nitrogen and oxygen, wherein at least one of the heteroatoms is nitrogen; each X is independently halogen, C₁₋₅alkyl, —NH₂, or C₁₋₅alkoxy; n is an integer from 1-3; R₁ and R₂ are each independently H or C₁₋₅alkyl; L₁ is absent or alkylene; R₃ is hydrogen, alkyl, or cycloalkyl; R₆ is alkyl, alkoxy, cycloalkyl, or heterocyclyl; R₈ and R₉ are each independently H, F, or alkyl; R₁₀ is H, F, or -L₂-R₇, wherein L₂ is absent or alkylene, and R₇ is H, alkyl, —O-alkyl, cycloalkyl, or heterocyclyl; and Ru is H, F, or alkyl.

In some embodiments of Formula (I), is:

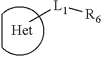

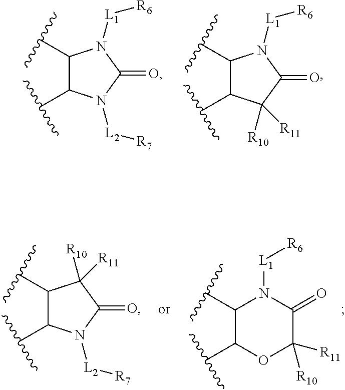

each X is independently halogen, C₁₋₅alkyl, —NH₂, or —O—C₁₋₅alkyl; n is an integer from 1-3; R₁ and R₂ are each independently H or C₁₋₃alkyl; L₁ is absent or C₁₋₅alkylene; R₃ is C₁₋₃alkyl; R₆ is C₁₋₅alkyl, —O—C₁₋₅alkyl, C₃₋₆cycloalkyl, or 3- to 6-membered heterocyclyl; R₁₀ is H, F, or -L₂-R₇, wherein L₂ is absent or C₁₋₅alkylene, and R₇ is H, C₁₋₅alkyl, —O—C₁₋₅alkyl, C₃₋₆cycloalkyl, or 4- to 6-membered heterocyclyl; and R₁₁ is H, F, or C₁₋₅alkyl, or an R₁₀ and R₁₁ taken together with the carbon atom to which they are attached form a C₃₋₆cycloalkyl or a 4- to 7-membered heterocyclyl as defined herein.

In some embodiments of Formula (I),

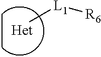

is:

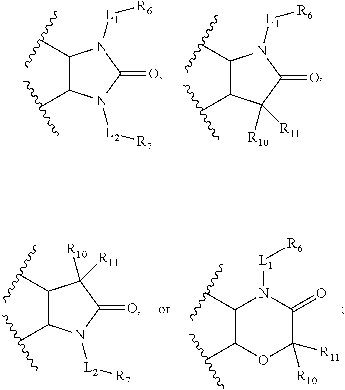

each X is independently —CF₂CH₃, —CF₂CH₂OH, —CF₂C(CH₃)₂OH, —CHF₂, —CF₃, F, or —NH₂; n is an integer from 1-3; R₁ and R₂ are each independently H or C₁₋₃alkyl; L₁ is absent or C₁₋₃alkylene; R₃ is methyl; R₆ is C₁₋₅alkyl, C₃₋₆cycloalkyl, or 4- to 6-membered heterocyclyl; R₁₀ is -L₂-R₇, wherein L₂ is absent or C₁₋₃alkylene, and R₇ is C₁₋₅alkyl; and R₁₁ is H, F, or C₁₋₅alkyl, or an R₁₀ and R₁₁ taken together with the carbon atom to which they are attached form a C₃₋₆cycloalkyl or a 4- to 7-membered heterocyclyl as defined herein.

In some embodiments, the present disclosure provides a compound of Formula (Ia):

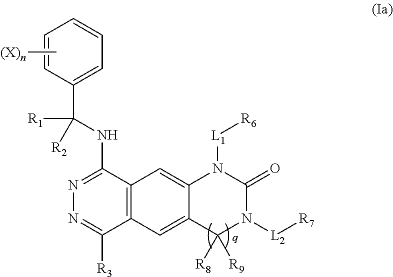

or a pharmaceutically acceptable salt thereof, wherein X, R₁, R₂, R₃, R₆, R₇, R₈, R₉, L₁, L₂, n and q are as defined herein.

In some embodiments, the present disclosure provides a compound of Formula (Ia-1):

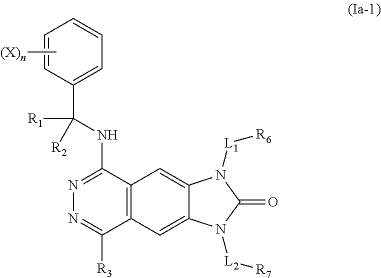

or a pharmaceutically acceptable salt thereof, wherein X, R₁, R₂, R₃, R₆, R₇, L₁, L₂, and n are as defined herein.

In some embodiments, the present disclosure provides a compound of Formula (Ib):

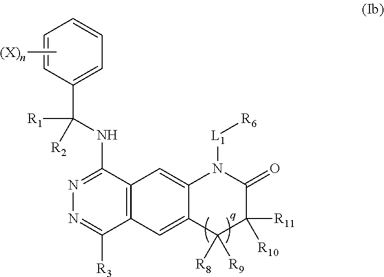

or a pharmaceutically acceptable salt thereof, wherein X, R₁, R₂, R₃, R₆, R₈, R₉, R₁₀, Rn, La, n and q are as defined herein.

In some embodiments of Formula (Ia) and Formula (Ib), q is 0. In some embodiments, q is 1.

In some embodiments, the present disclosure provides a compound of Formula (Ib-1):

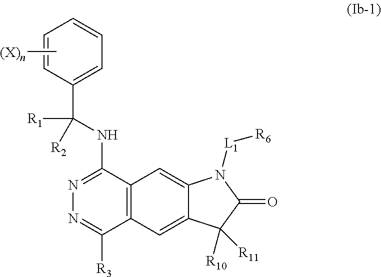

or a pharmaceutically acceptable salt thereof, wherein X, R₁, R₂, R₃, R₆, R₁₀, R₁₁, L₁, and n are as defined herein.

In some embodiments, the present disclosure provides a compound of Formula (Ic):

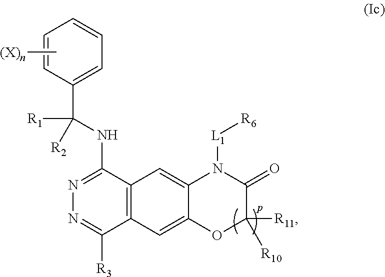

or a pharmaceutically acceptable salt thereof, wherein X, R₁, R₂, R₃, R₆, R₁₀, R₁₁, L₁, n, and p are as defined herein.

In some embodiments, the present disclosure provides a compound of Formula (Ic-1):

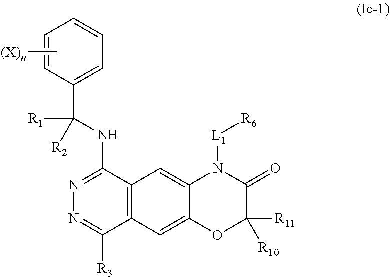

or a pharmaceutically acceptable salt thereof, wherein X, R₁, R₂, R₃, R₆, R₁₀, R₁₁, L₁, and n are as defined herein.

In some embodiments, the present disclosure provides a compound of Formula (Id-1):

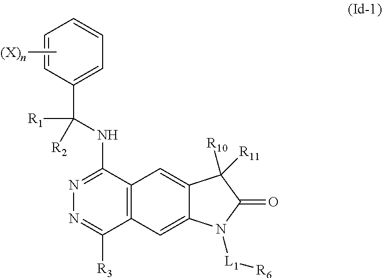

or a pharmaceutically acceptable salt thereof, wherein X, R₁, R₂, R₃, R₆, R₁₀, R₁₁, L₁, and n are as defined herein.

In some embodiments, the compound of the present disclosure is:

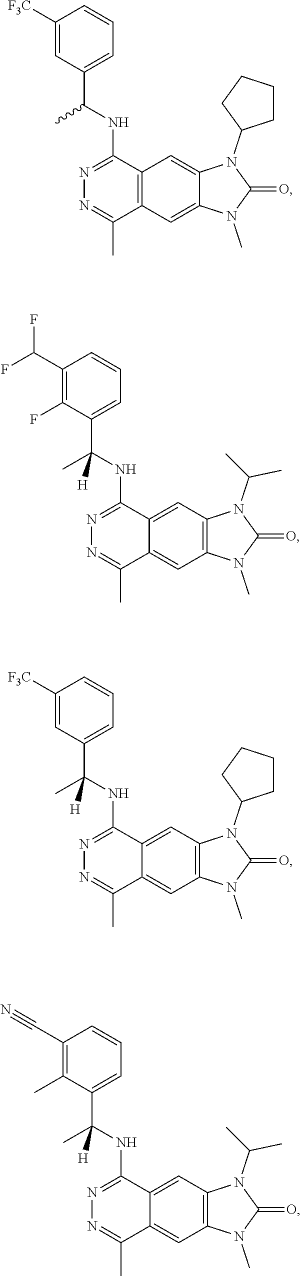 [SMILES: Cc1nnc(NC(C)c2cccc(C(F)(F)F)c2)c2cc3c(cc12)n(C)c(=O)n3C1CCCC1 ; Cc1nnc(N[C@H](C)c2cccc(C(F)F)c2F)c2cc3c(cc12)n(C)c(=O)n3C(C)C ; Cc1nnc(N[C@H](C)c2cccc(C(F)(F)F)c2)c2cc3c(cc12)n(C)c(=O)n3C1CCCC1 ; Cc1c(C#N)cccc1[C@@H](C)Nc1nnc(C)c2cc3c(cc12)n(C(C)C)c(=O)n3C]

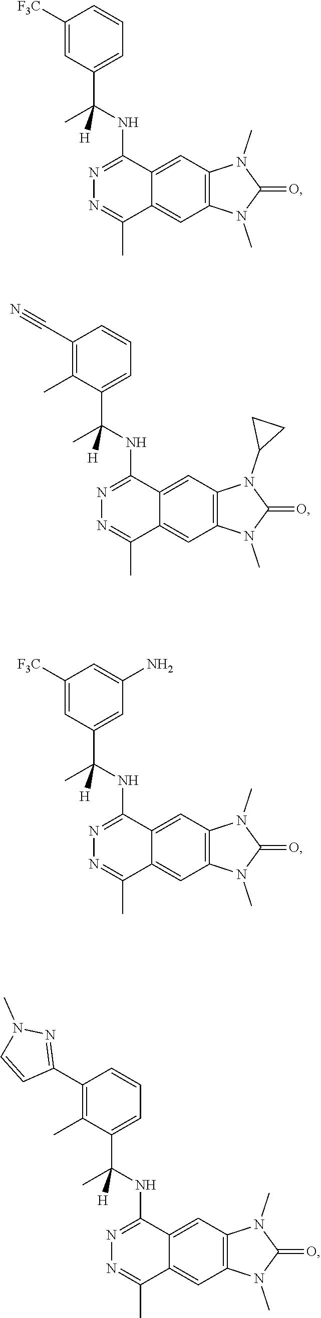 [SMILES: Cc1nnc(N[C@H](C)c2cccc(C(F)(F)F)c2)c2cc3c(cc12)n(C)c(=O)n3C ; Cc1c(C#N)cccc1[C@@H](C)Nc1nnc(C)c2cc3c(cc12)n(C1CC1)c(=O)n3C ; Cc1nnc(N[C@H](C)c2cc(N)cc(C(F)(F)F)c2)c2cc3c(cc12)n(C)c(=O)n3C ; Cc1c(-c2ccn(C)n2)cccc1[C@@H](C)Nc1nnc(C)c2cc3c(cc12)n(C)c(=O)n3C]

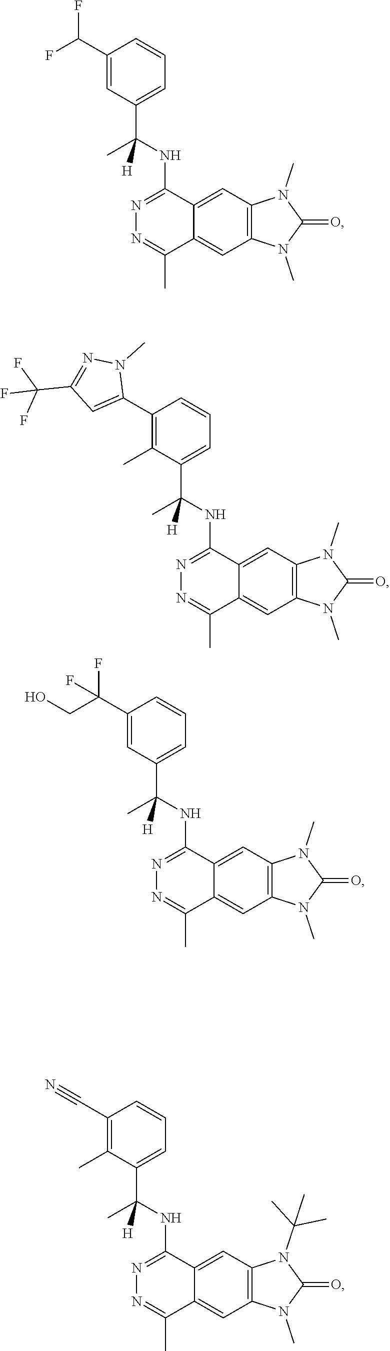 [SMILES: Cc1nnc(N[C@H](C)c2cccc(C(F)F)c2)c2cc3c(cc12)n(C)c(=O)n3C ; Cc1c(-c2cc(C(F)(F)F)nn2C)cccc1[C@@H](C)Nc1nnc(C)c2cc3c(cc12)n(C)c(=O)n3C ; Cc1nnc(N[C@H](C)c2cccc(C(F)(F)CO)c2)c2cc3c(cc12)n(C)c(=O)n3C ; Cc1c(C#N)cccc1[C@@H](C)Nc1nnc(C)c2cc3c(cc12)n(C(C)(C)C)c(=O)n3C]

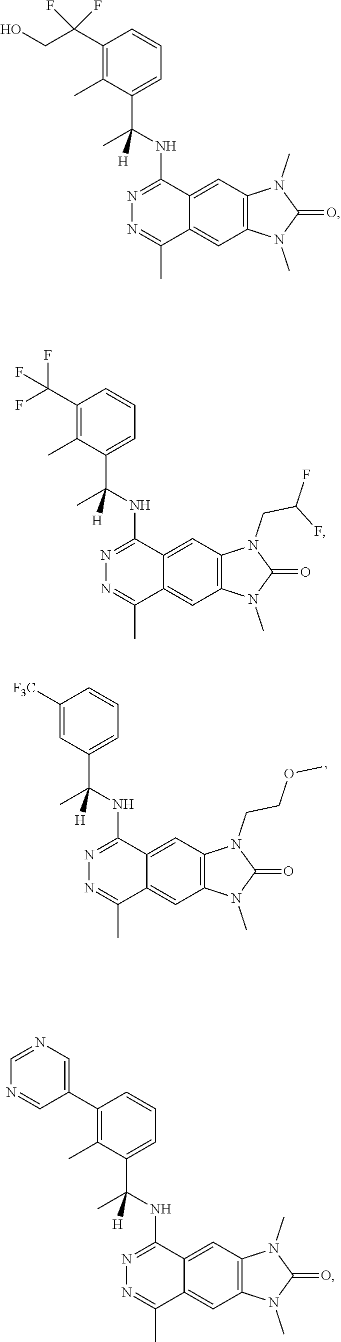 [SMILES: Cc1c([C@@H](C)Nc2nnc(C)c3cc4c(cc23)n(C)c(=O)n4C)cccc1C(F)(F)CO ; Cc1c([C@@H](C)Nc2nnc(C)c3cc4c(cc23)n(CC(F)F)c(=O)n4C)cccc1C(F)(F)F ; COCCn1c(=O)n(C)c2cc3c(C)nnc(N[C@H](C)c4cccc(C(F)(F)F)c4)c3cc21 ; Cc1c(-c2cncnc2)cccc1[C@@H](C)Nc1nnc(C)c2cc3c(cc12)n(C)c(=O)n3C]

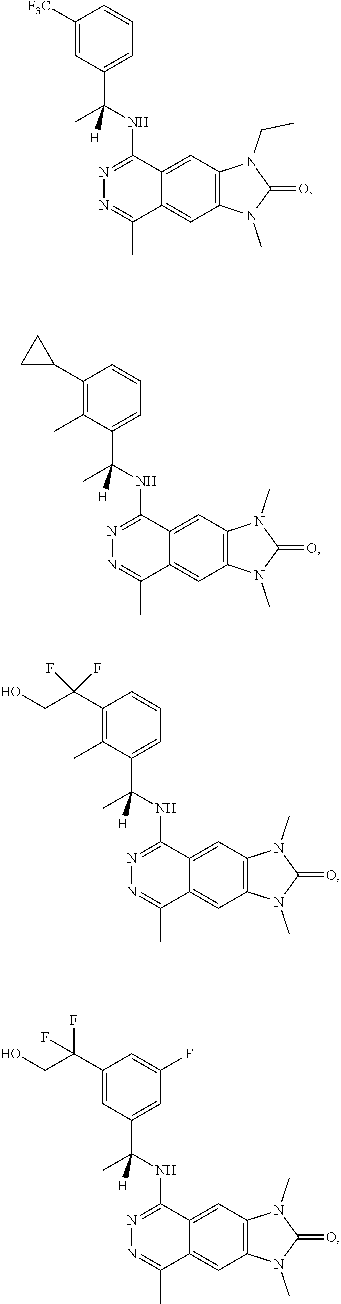 [SMILES: CCn1c(=O)n(C)c2cc3c(C)nnc(N[C@H](C)c4cccc(C(F)(F)F)c4)c3cc21 ; Cc1c(C2CC2)cccc1[C@@H](C)Nc1nnc(C)c2cc3c(cc12)n(C)c(=O)n3C ; Cc1c([C@@H](C)Nc2nnc(C)c3cc4c(cc23)n(C)c(=O)n4C)cccc1C(F)(F)CO ; Cc1nnc(N[C@H](C)c2cc(F)cc(C(F)(F)CO)c2)c2cc3c(cc12)n(C)c(=O)n3C]

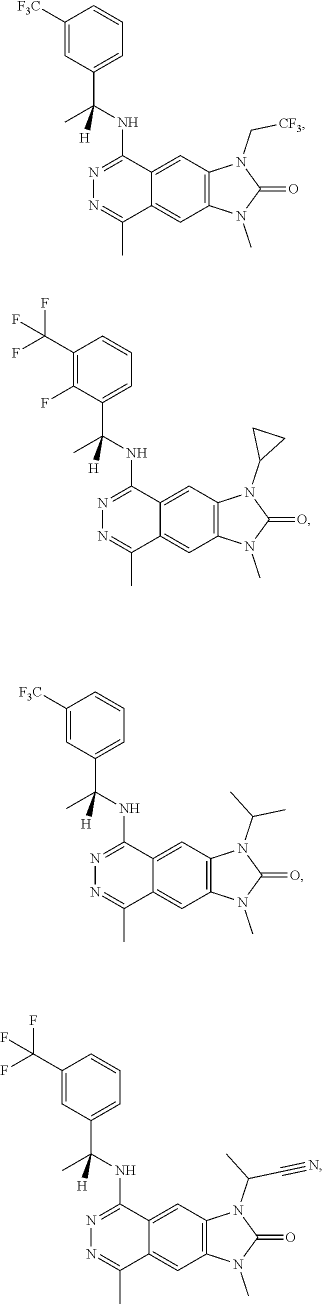 [SMILES: Cc1nnc(N[C@H](C)c2cccc(C(F)(F)F)c2)c2cc3c(cc12)n(C)c(=O)n3CC(F)(F)F ; Cc1nnc(N[C@H](C)c2cccc(C(F)(F)F)c2F)c2cc3c(cc12)n(C)c(=O)n3C1CC1 ; Cc1nnc(N[C@H](C)c2cccc(C(F)(F)F)c2)c2cc3c(cc12)n(C)c(=O)n3C(C)C ; Cc1nnc(N[C@H](C)c2cccc(C(F)(F)F)c2)c2cc3c(cc12)n(C)c(=O)n3C(C)C#N]

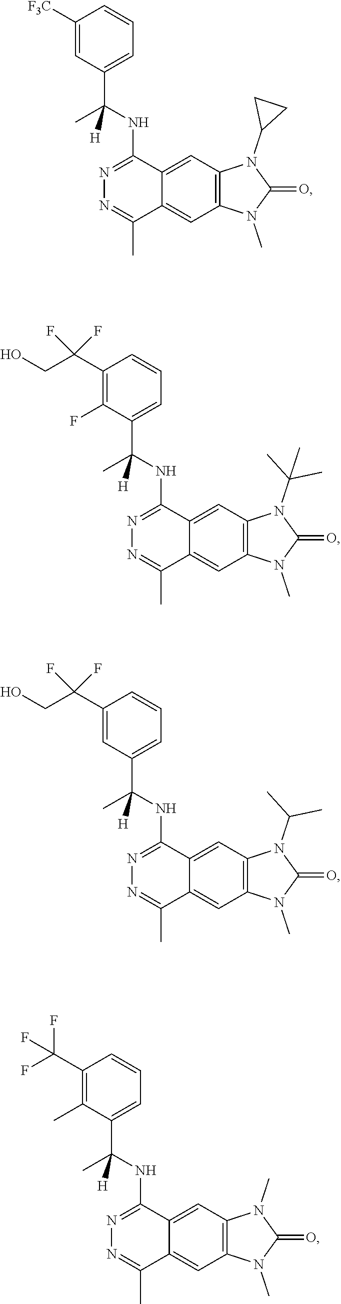 [SMILES: Cc1nnc(N[C@H](C)c2cccc(C(F)(F)F)c2)c2cc3c(cc12)n(C)c(=O)n3C1CC1 ; Cc1nnc(N[C@H](C)c2cccc(C(F)(F)CO)c2F)c2cc3c(cc12)n(C)c(=O)n3C(C)(C)C ; Cc1nnc(N[C@H](C)c2cccc(C(F)(F)CO)c2)c2cc3c(cc12)n(C)c(=O)n3C(C)C ; Cc1c([C@@H](C)Nc2nnc(C)c3cc4c(cc23)n(C)c(=O)n4C)cccc1C(F)(F)F]

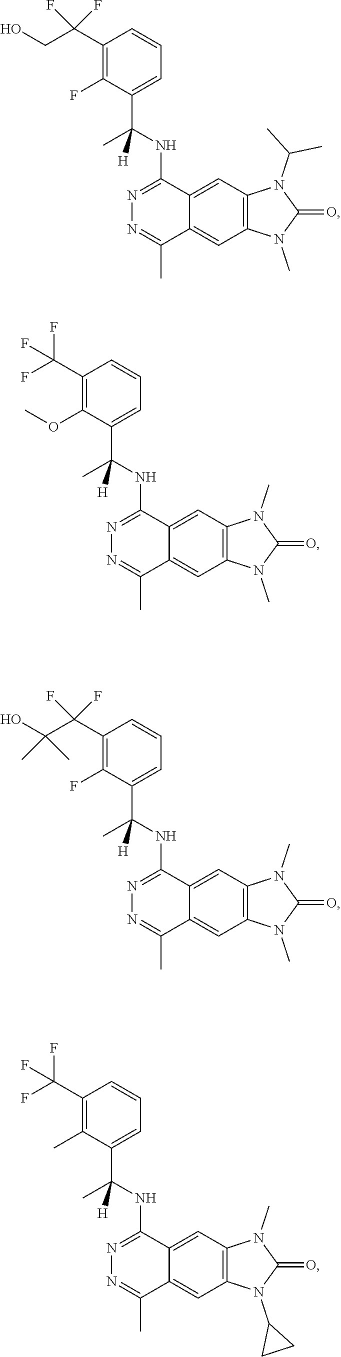 [SMILES: Cc1nnc(N[C@H](C)c2cccc(C(F)(F)CO)c2F)c2cc3c(cc12)n(C)c(=O)n3C(C)C ; COc1c([C@@H](C)Nc2nnc(C)c3cc4c(cc23)n(C)c(=O)n4C)cccc1C(F)(F)F ; Cc1nnc(N[C@H](C)c2cccc(C(F)(F)C(C)(C)O)c2F)c2cc3c(cc12)n(C)c(=O)n3C ; Cc1c([C@@H](C)Nc2nnc(C)c3cc4c(cc23)n(C)c(=O)n4C2CC2)cccc1C(F)(F)F]

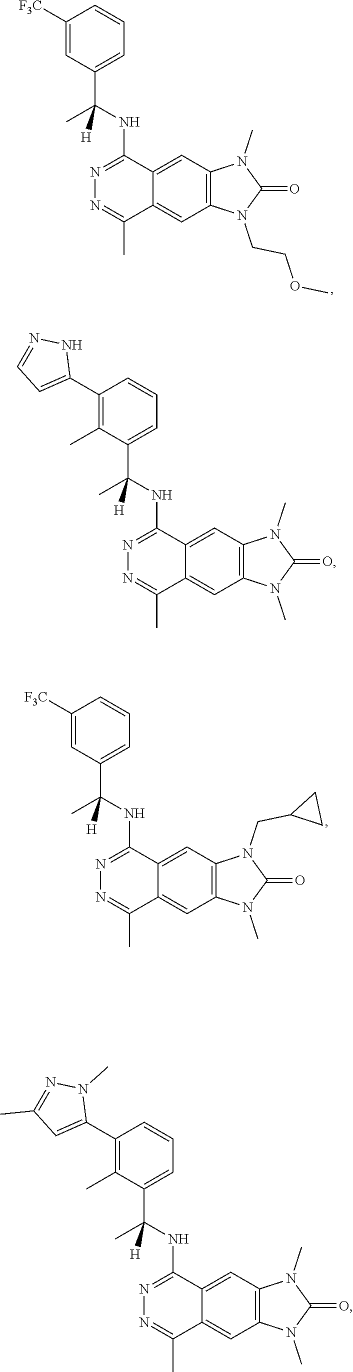

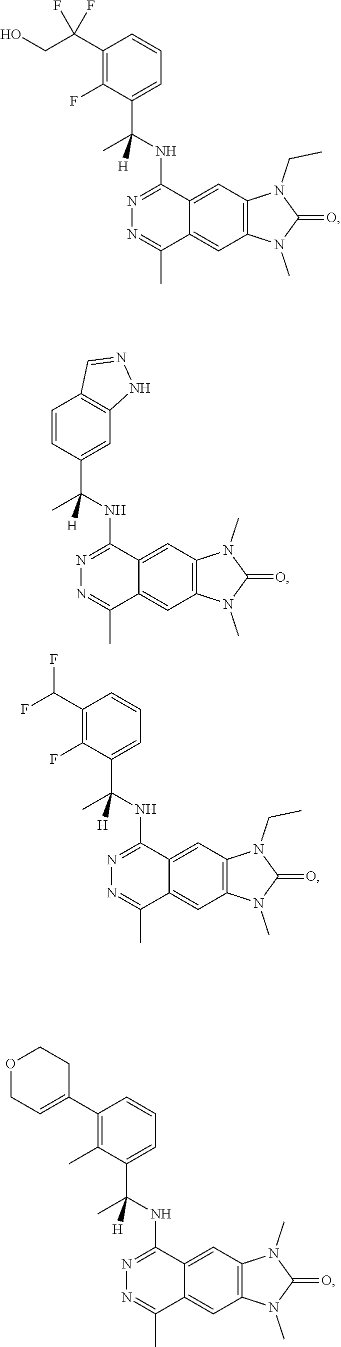 [SMILES: CCn1c(=O)n(C)c2cc3c(C)nnc(N[C@H](C)c4cccc(C(F)(F)CO)c4F)c3cc21 ; Cc1nnc(N[C@H](C)c2ccc3cn[nH]c3c2)c2cc3c(cc12)n(C)c(=O)n3C ; CCn1c(=O)n(C)c2cc3c(C)nnc(N[C@H](C)c4cccc(C(F)F)c4F)c3cc21 ; Cc1c(C2=CCOCC2)cccc1[C@@H](C)Nc1nnc(C)c2cc3c(cc12)n(C)c(=O)n3C]

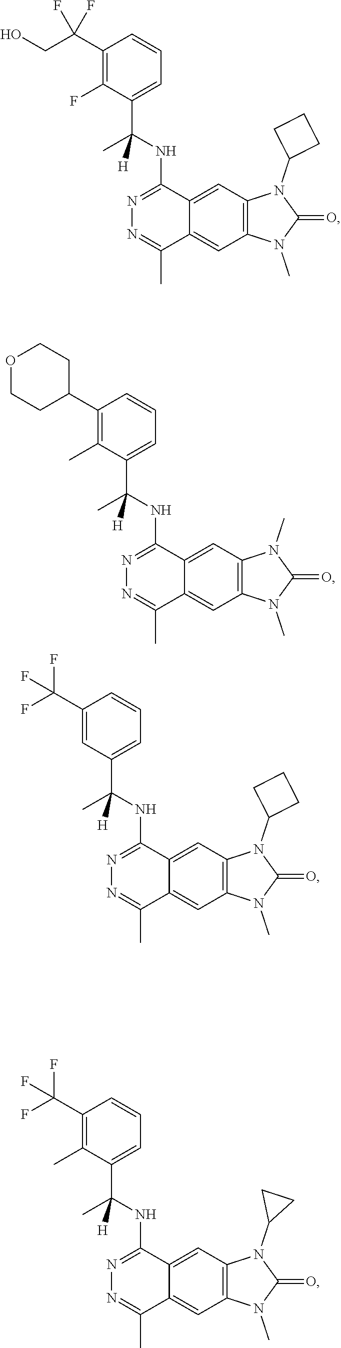 [SMILES: Cc1nnc(N[C@H](C)c2cccc(C(F)(F)CO)c2F)c2cc3c(cc12)n(C)c(=O)n3C1CCC1 ; Cc1c(C2CCOCC2)cccc1[C@@H](C)Nc1nnc(C)c2cc3c(cc12)n(C)c(=O)n3C ; Cc1nnc(N[C@H](C)c2cccc(C(F)(F)F)c2)c2cc3c(cc12)n(C)c(=O)n3C1CCC1 ; Cc1c([C@@H](C)Nc2nnc(C)c3cc4c(cc23)n(C2CC2)c(=O)n4C)cccc1C(F)(F)F]

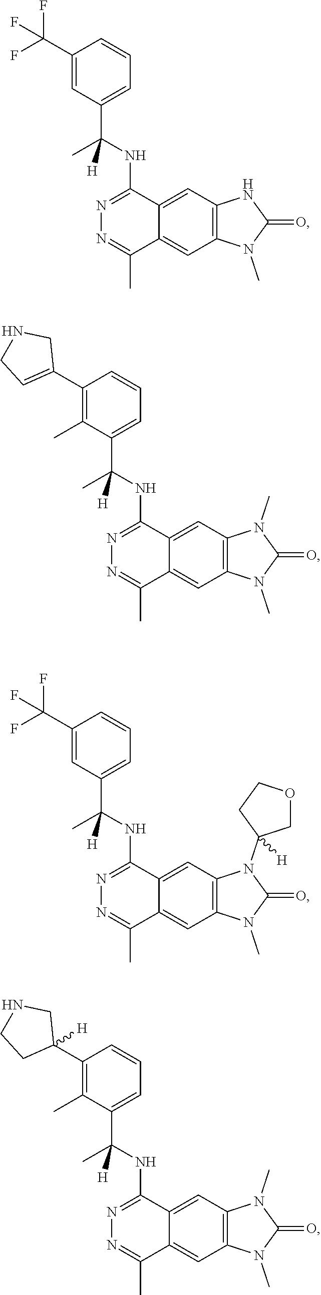 [SMILES: Cc1nnc(N[C@H](C)c2cccc(C(F)(F)F)c2)c2cc3[nH]c(=O)n(C)c3cc12 ; Cc1c(C2=CCNC2)cccc1[C@@H](C)Nc1nnc(C)c2cc3c(cc12)n(C)c(=O)n3C ; Cc1nnc(N[C@H](C)c2cccc(C(F)(F)F)c2)c2cc3c(cc12)n(C)c(=O)n3C1CCOC1 ; Cc1c(C2CCNC2)cccc1[C@@H](C)Nc1nnc(C)c2cc3c(cc12)n(C)c(=O)n3C]

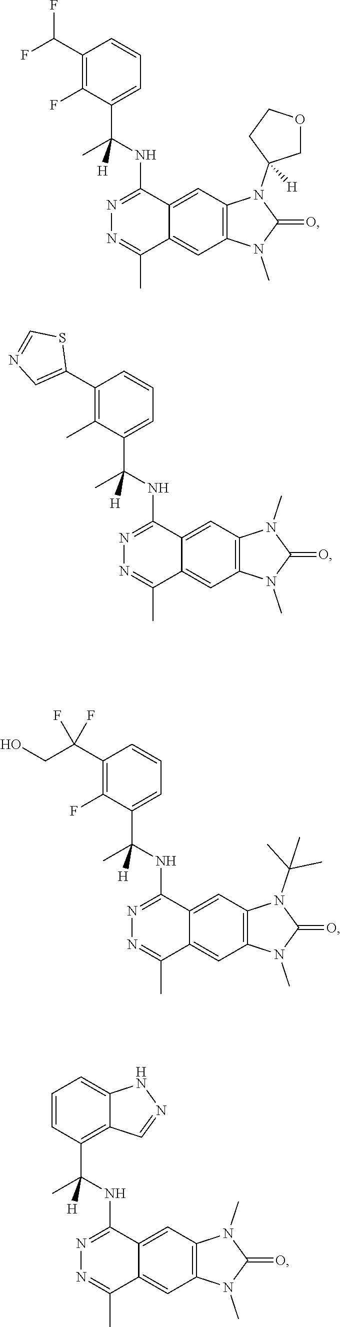 [SMILES: Cc1nnc(N[C@H](C)c2cccc(C(F)F)c2F)c2cc3c(cc12)n(C)c(=O)n3[C@H]1CCOC1 ; Cc1c(-c2cncs2)cccc1[C@@H](C)Nc1nnc(C)c2cc3c(cc12)n(C)c(=O)n3C ; Cc1nnc(N[C@H](C)c2cccc(C(F)(F)CO)c2F)c2cc3c(cc12)n(C)c(=O)n3C(C)(C)C ; Cc1nnc(N[C@H](C)c2cccc3[nH]ncc23)c2cc3c(cc12)n(C)c(=O)n3C]

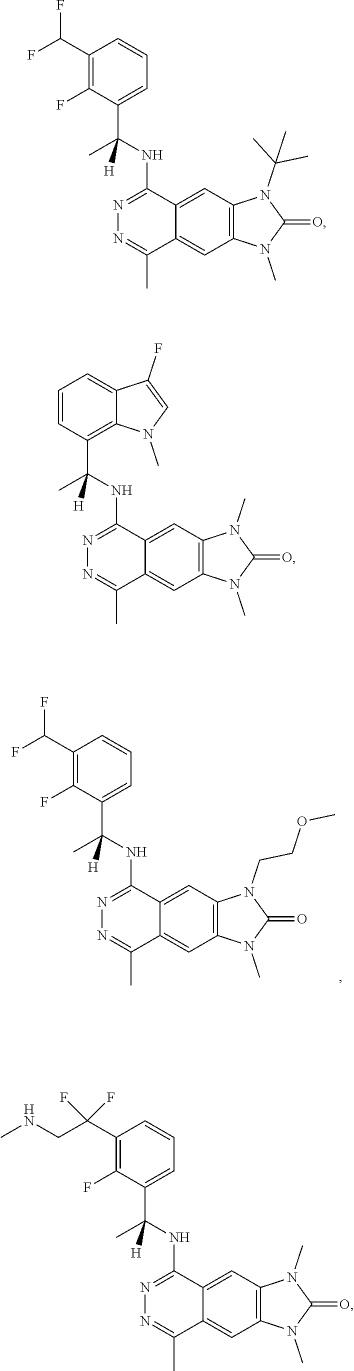

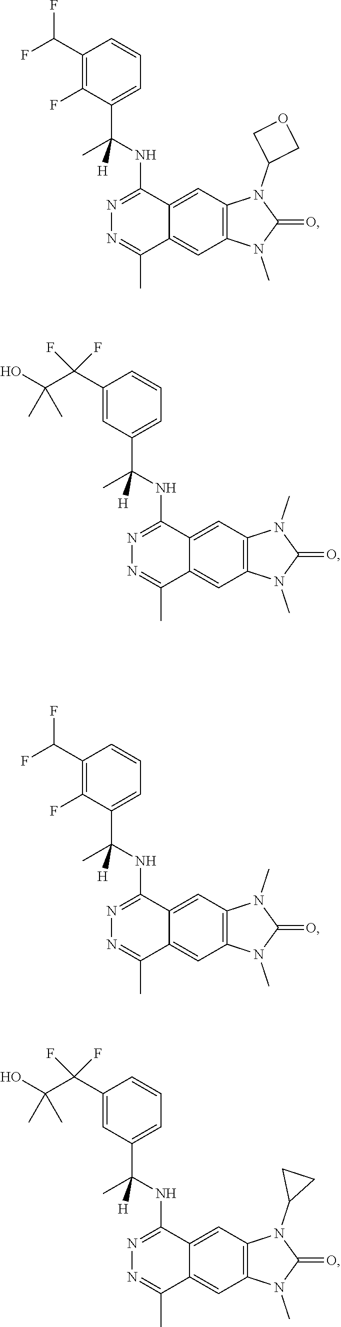 [SMILES: Cc1nnc(N[C@H](C)c2cccc(C(F)F)c2F)c2cc3c(cc12)n(C)c(=O)n3C1COC1 ; Cc1nnc(N[C@H](C)c2cccc(C(F)(F)C(C)(C)O)c2)c2cc3c(cc12)n(C)c(=O)n3C ; Cc1nnc(N[C@H](C)c2cccc(C(F)F)c2F)c2cc3c(cc12)n(C)c(=O)n3C ; Cc1nnc(N[C@H](C)c2cccc(C(F)(F)C(C)(C)O)c2)c2cc3c(cc12)n(C)c(=O)n3C1CC1]

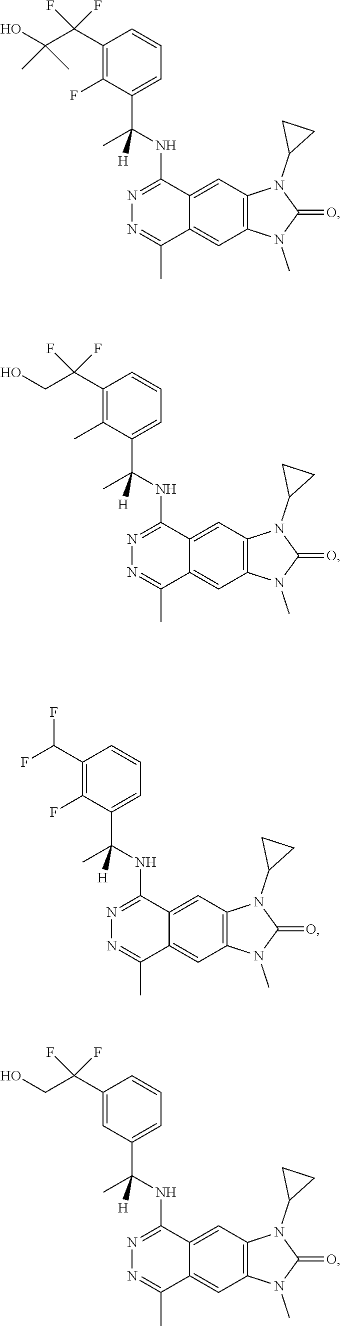 [SMILES: Cc1nnc(N[C@H](C)c2cccc(C(F)(F)C(C)(C)O)c2F)c2cc3c(cc12)n(C)c(=O)n3C1CC1 ; Cc1c([C@@H](C)Nc2nnc(C)c3cc4c(cc23)n(C2CC2)c(=O)n4C)cccc1C(F)(F)CO ; Cc1nnc(N[C@H](C)c2cccc(C(F)F)c2F)c2cc3c(cc12)n(C)c(=O)n3C1CC1 ; Cc1nnc(N[C@H](C)c2cccc(C(F)(F)CO)c2)c2cc3c(cc12)n(C)c(=O)n3C1CC1]

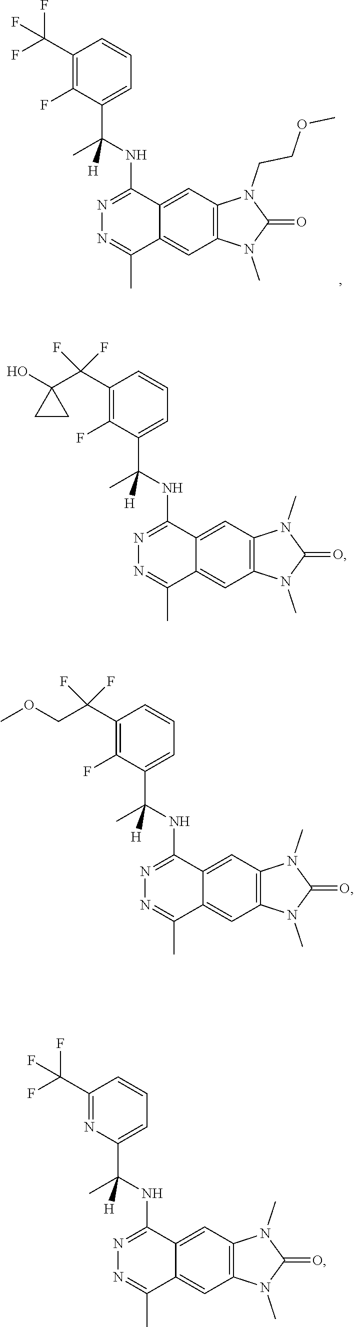

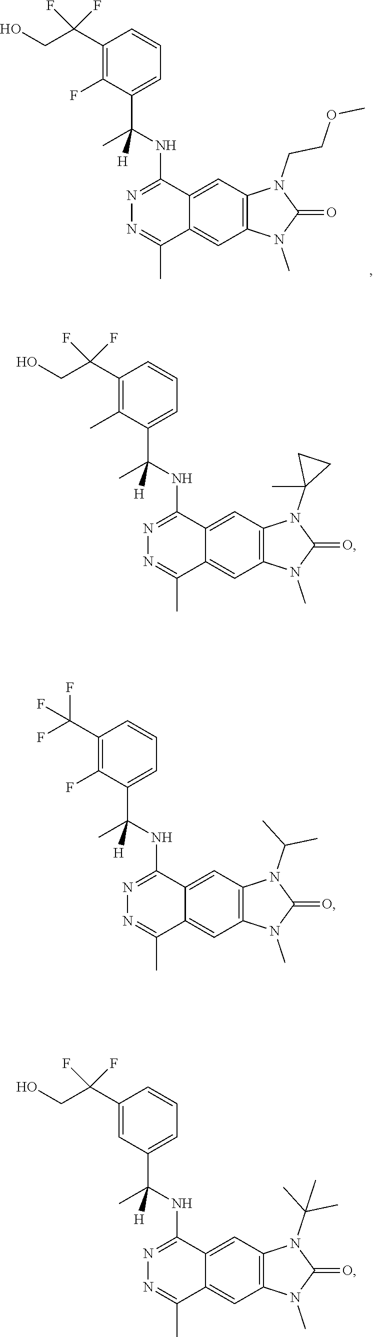 [SMILES: COCCn1c(=O)n(C)c2cc3c(C)nnc(N[C@H](C)c4cccc(C(F)(F)CO)c4F)c3cc21 ; Cc1c([C@@H](C)Nc2nnc(C)c3cc4c(cc23)n(C2(C)CC2)c(=O)n4C)cccc1C(F)(F)CO ; Cc1nnc(N[C@H](C)c2cccc(C(F)(F)F)c2F)c2cc3c(cc12)n(C)c(=O)n3C(C)C ; Cc1nnc(N[C@H](C)c2cccc(C(F)(F)CO)c2)c2cc3c(cc12)n(C)c(=O)n3C(C)(C)C]

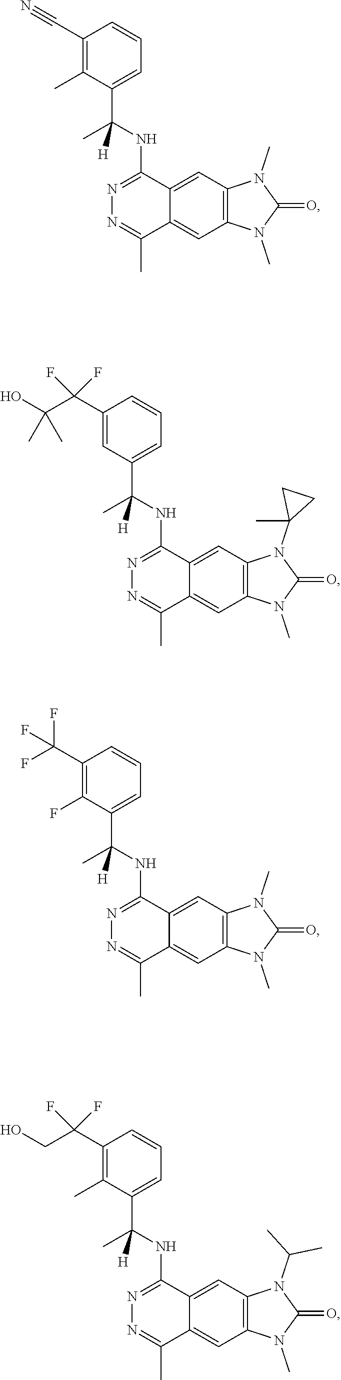 [SMILES: Cc1c(C#N)cccc1[C@@H](C)Nc1nnc(C)c2cc3c(cc12)n(C)c(=O)n3C ; Cc1nnc(N[C@H](C)c2cccc(C(F)(F)C(C)(C)O)c2)c2cc3c(cc12)n(C)c(=O)n3C1(C)CC1 ; Cc1nnc(N[C@H](C)c2cccc(C(F)(F)F)c2F)c2cc3c(cc12)n(C)c(=O)n3C ; Cc1c([C@@H](C)Nc2nnc(C)c3cc4c(cc23)n(C(C)C)c(=O)n4C)cccc1C(F)(F)CO]

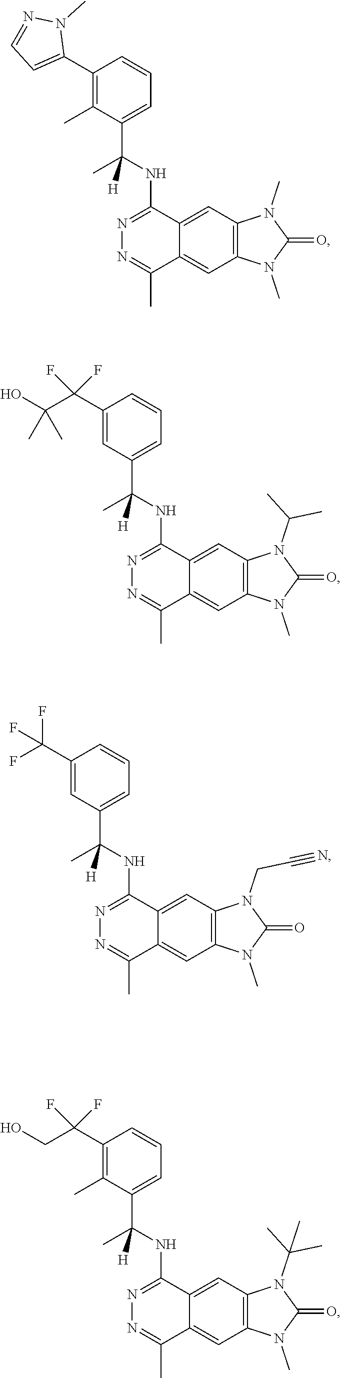 [SMILES: Cc1c(-c2ccnn2C)cccc1[C@@H](C)Nc1nnc(C)c2cc3c(cc12)n(C)c(=O)n3C ; Cc1nnc(N[C@H](C)c2cccc(C(F)(F)C(C)(C)O)c2)c2cc3c(cc12)n(C)c(=O)n3C(C)C ; Cc1nnc(N[C@H](C)c2cccc(C(F)(F)F)c2)c2cc3c(cc12)n(C)c(=O)n3CC#N ; Cc1c([C@@H](C)Nc2nnc(C)c3cc4c(cc23)n(C(C)(C)C)c(=O)n4C)cccc1C(F)(F)CO]

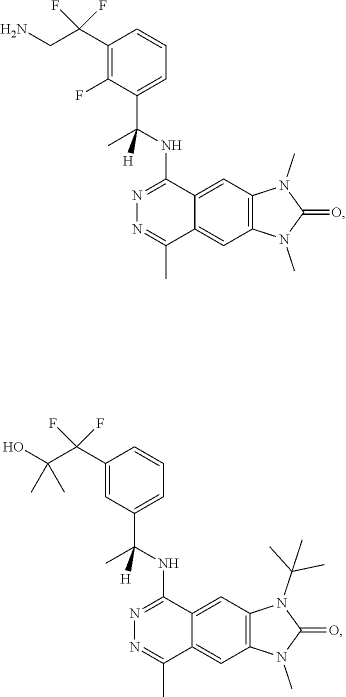 [SMILES: Cc1nnc(N[C@H](C)c2cccc(C(F)(F)CN)c2F)c2cc3c(cc12)n(C)c(=O)n3C ; Cc1nnc(N[C@H](C)c2cccc(C(F)(F)C(C)(C)O)c2)c2cc3c(cc12)n(C)c(=O)n3C(C)(C)C]

or a pharmaceutically acceptable salt thereof.

In some embodiments, the compound of the present disclosure is

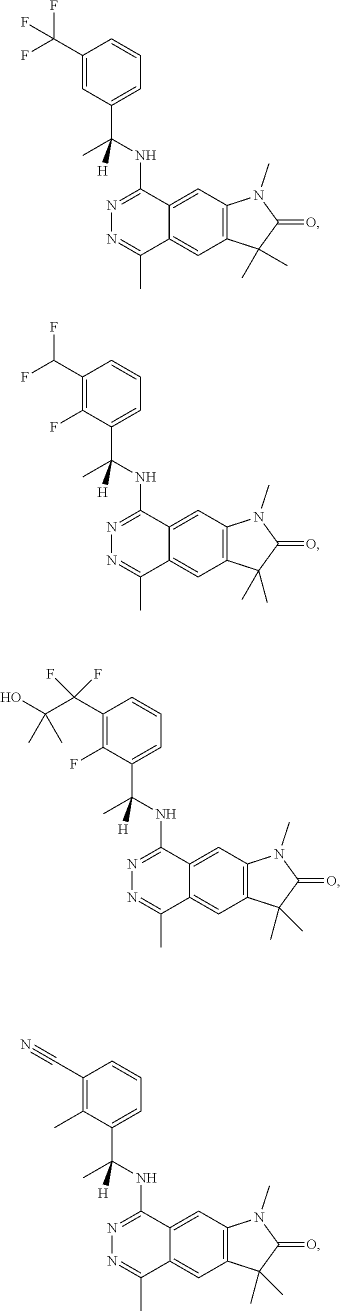 [SMILES: Cc1nnc(N[C@H](C)c2cccc(C(F)(F)F)c2)c2cc3c(cc12)C(C)(C)C(=O)N3C ; Cc1nnc(N[C@H](C)c2cccc(C(F)F)c2F)c2cc3c(cc12)C(C)(C)C(=O)N3C ; Cc1nnc(N[C@H](C)c2cccc(C(F)(F)C(C)(C)O)c2F)c2cc3c(cc12)C(C)(C)C(=O)N3C ; Cc1c(C#N)cccc1[C@@H](C)Nc1nnc(C)c2cc3c(cc12)N(C)C(=O)C3(C)C]

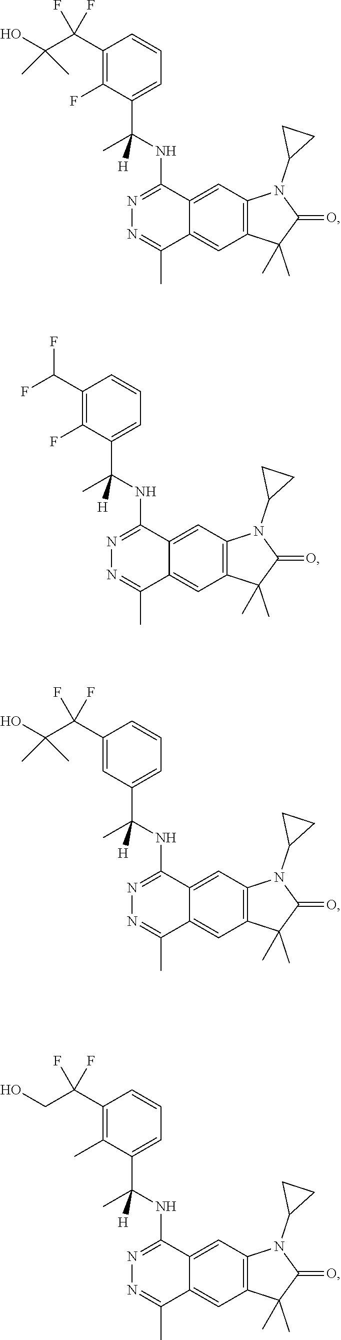 [SMILES: Cc1nnc(N[C@H](C)c2cccc(C(F)(F)C(C)(C)O)c2F)c2cc3c(cc12)C(C)(C)C(=O)N3C1CC1 ; Cc1nnc(N[C@H](C)c2cccc(C(F)F)c2F)c2cc3c(cc12)C(C)(C)C(=O)N3C1CC1 ; Cc1nnc(N[C@H](C)c2cccc(C(F)(F)C(C)(C)O)c2)c2cc3c(cc12)C(C)(C)C(=O)N3C1CC1 ; Cc1c([C@@H](C)Nc2nnc(C)c3cc4c(cc23)N(C2CC2)C(=O)C4(C)C)cccc1C(F)(F)CO]

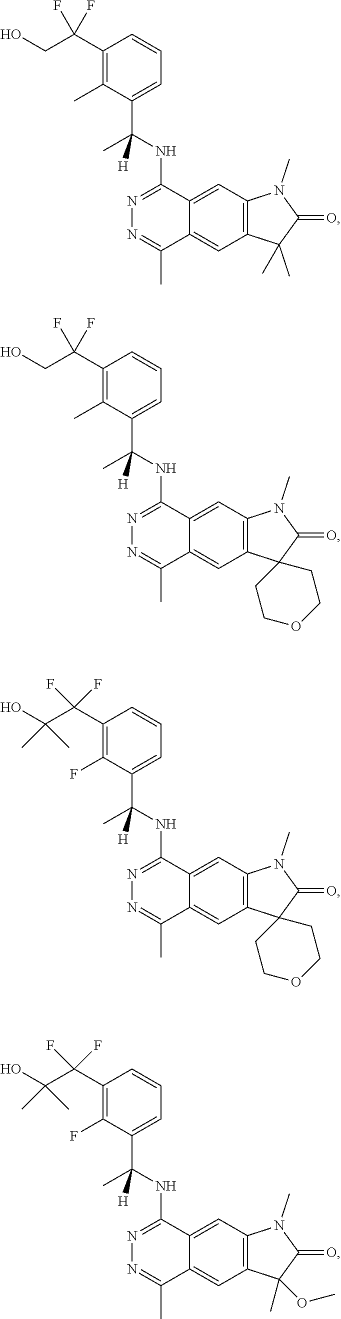 [SMILES: Cc1c([C@@H](C)Nc2nnc(C)c3cc4c(cc23)N(C)C(=O)C4(C)C)cccc1C(F)(F)CO ; Cc1c([C@@H](C)Nc2nnc(C)c3cc4c(cc23)N(C)C(=O)C42CCOCC2)cccc1C(F)(F)CO ; Cc1nnc(N[C@H](C)c2cccc(C(F)(F)C(C)(C)O)c2F)c2cc3c(cc12)C1(CCOCC1)C(=O)N3C ; COC1(C)C(=O)N(C)c2cc3c(N[C@H](C)c4cccc(C(F)(F)C(C)(C)O)c4F)nnc(C)c3cc21]

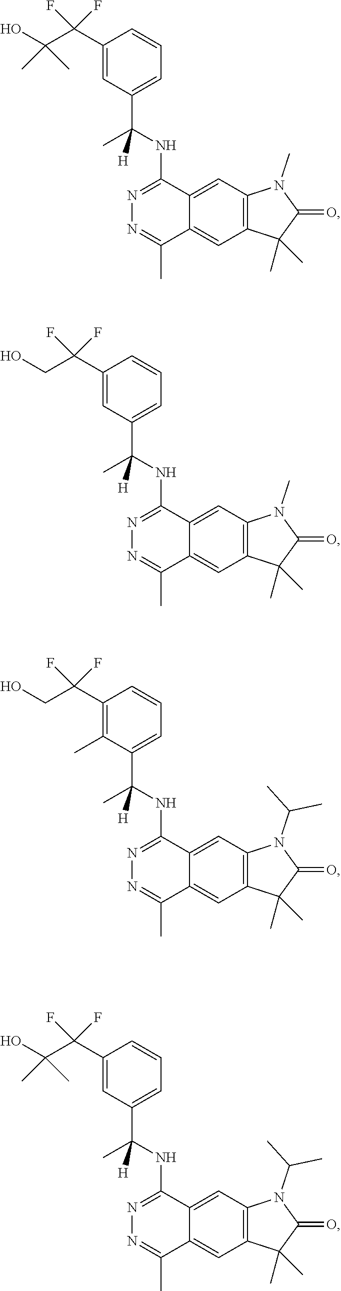 [SMILES: Cc1nnc(N[C@H](C)c2cccc(C(F)(F)C(C)(C)O)c2)c2cc3c(cc12)C(C)(C)C(=O)N3C ; Cc1nnc(N[C@H](C)c2cccc(C(F)(F)CO)c2)c2cc3c(cc12)C(C)(C)C(=O)N3C ; Cc1c([C@@H](C)Nc2nnc(C)c3cc4c(cc23)N(C(C)C)C(=O)C4(C)C)cccc1C(F)(F)CO ; Cc1nnc(N[C@H](C)c2cccc(C(F)(F)C(C)(C)O)c2)c2cc3c(cc12)C(C)(C)C(=O)N3C(C)C]

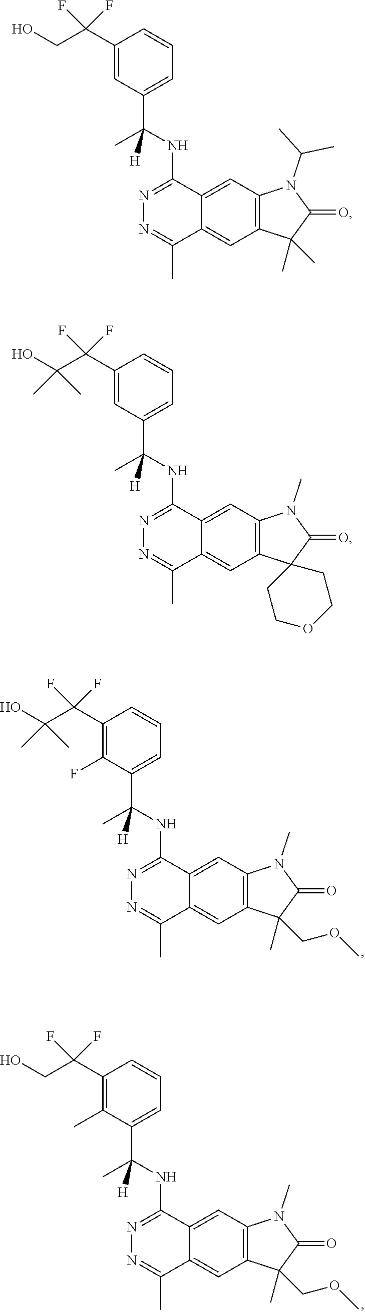 [SMILES: Cc1nnc(N[C@H](C)c2cccc(C(F)(F)CO)c2)c2cc3c(cc12)C(C)(C)C(=O)N3C(C)C ; Cc1nnc(N[C@H](C)c2cccc(C(F)(F)C(C)(C)O)c2)c2cc3c(cc12)C1(CCOCC1)C(=O)N3C ; COCC1(C)C(=O)N(C)c2cc3c(N[C@H](C)c4cccc(C(F)(F)C(C)(C)O)c4F)nnc(C)c3cc21 ; COCC1(C)C(=O)N(C)c2cc3c(N[C@H](C)c4cccc(C(F)(F)CO)c4C)nnc(C)c3cc21]

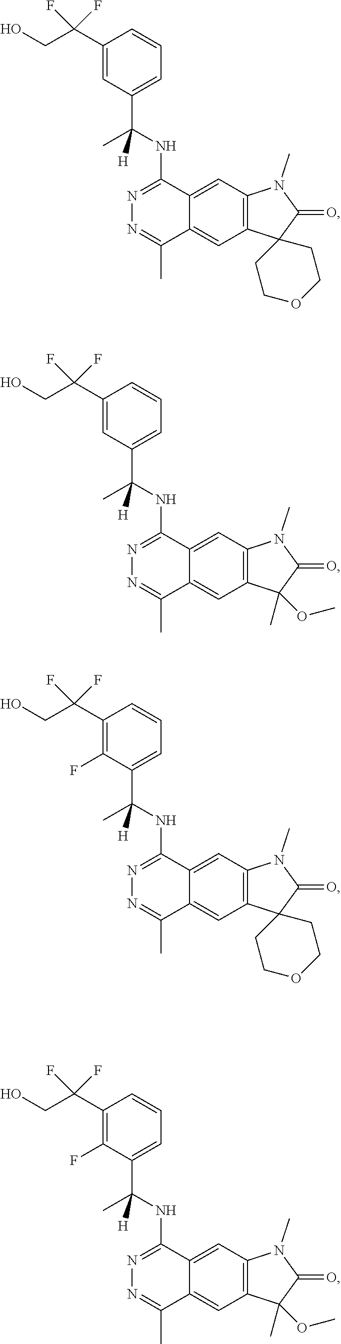 [SMILES: Cc1nnc(N[C@H](C)c2cccc(C(F)(F)CO)c2)c2cc3c(cc12)C1(CCOCC1)C(=O)N3C ; COC1(C)C(=O)N(C)c2cc3c(N[C@H](C)c4cccc(C(F)(F)CO)c4)nnc(C)c3cc21 ; Cc1nnc(N[C@H](C)c2cccc(C(F)(F)CO)c2F)c2cc3c(cc12)C1(CCOCC1)C(=O)N3C ; COC1(C)C(=O)N(C)c2cc3c(N[C@H](C)c4cccc(C(F)(F)CO)c4F)nnc(C)c3cc21]

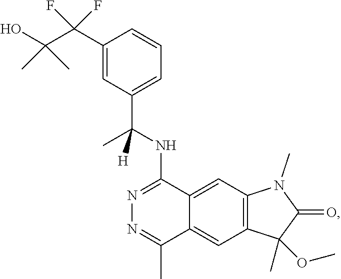 [SMILES: COC1(C)C(=O)N(C)c2cc3c(N[C@H](C)c4cccc(C(F)(F)C(C)(C)O)c4)nnc(C)c3cc21]

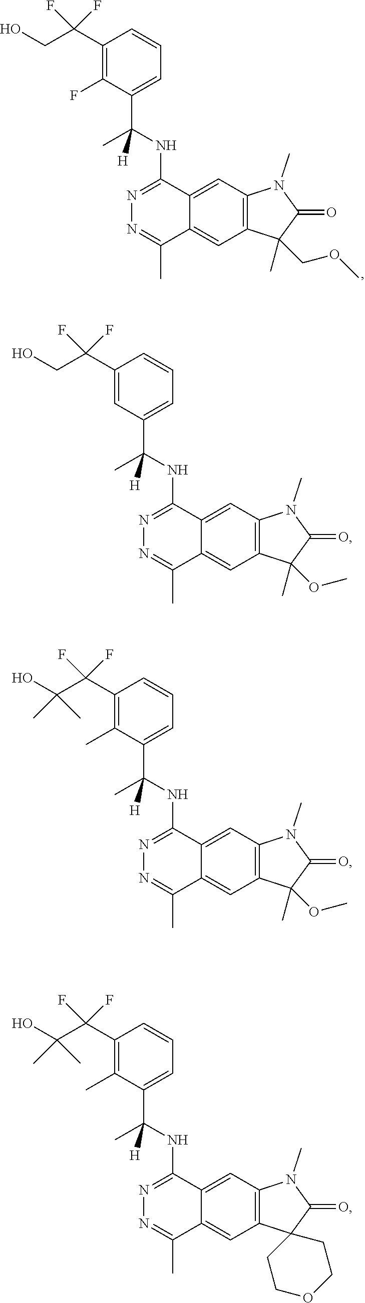 [SMILES: COCC1(C)C(=O)N(C)c2cc3c(N[C@H](C)c4cccc(C(F)(F)CO)c4F)nnc(C)c3cc21 ; COC1(C)C(=O)N(C)c2cc3c(N[C@H](C)c4cccc(C(F)(F)CO)c4)nnc(C)c3cc21 ; COC1(C)C(=O)N(C)c2cc3c(N[C@H](C)c4cccc(C(F)(F)C(C)(C)O)c4C)nnc(C)c3cc21 ; Cc1c([C@@H](C)Nc2nnc(C)c3cc4c(cc23)N(C)C(=O)C42CCOCC2)cccc1C(F)(F)C(C)(C)O]

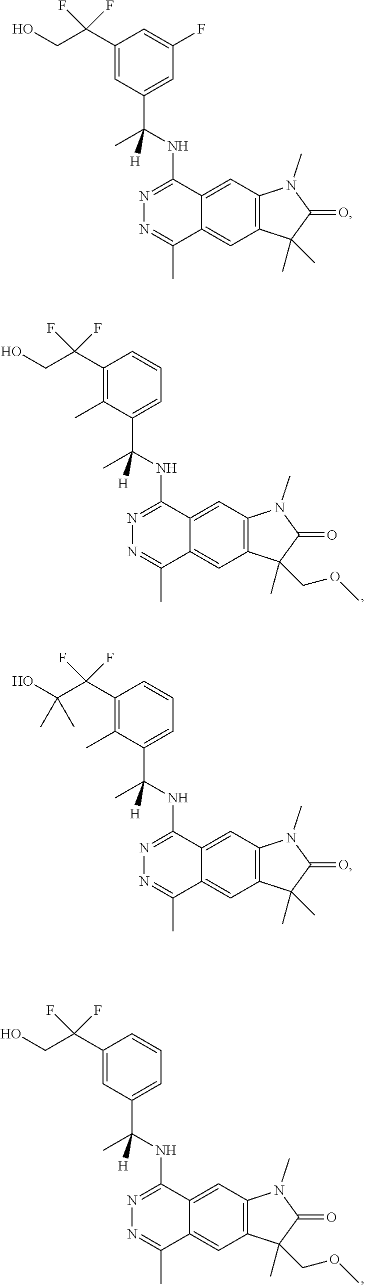 [SMILES: Cc1nnc(N[C@H](C)c2cc(F)cc(C(F)(F)CO)c2)c2cc3c(cc12)C(C)(C)C(=O)N3C ; COCC1(C)C(=O)N(C)c2cc3c(N[C@H](C)c4cccc(C(F)(F)CO)c4C)nnc(C)c3cc21 ; Cc1c([C@@H](C)Nc2nnc(C)c3cc4c(cc23)N(C)C(=O)C4(C)C)cccc1C(F)(F)C(C)(C)O ; COCC1(C)C(=O)N(C)c2cc3c(N[C@H](C)c4cccc(C(F)(F)CO)c4)nnc(C)c3cc21]

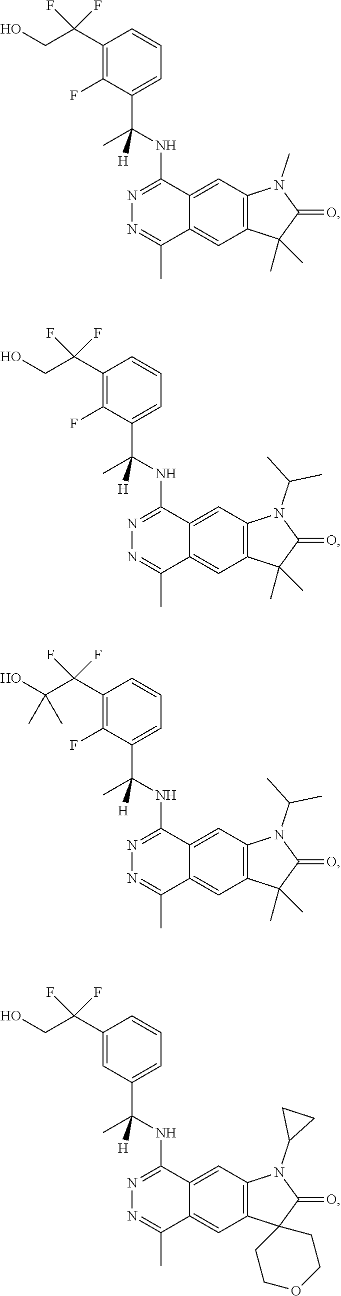 [SMILES: Cc1nnc(N[C@H](C)c2cccc(C(F)(F)CO)c2F)c2cc3c(cc12)C(C)(C)C(=O)N3C ; Cc1nnc(N[C@H](C)c2cccc(C(F)(F)CO)c2F)c2cc3c(cc12)C(C)(C)C(=O)N3C(C)C ; Cc1nnc(N[C@H](C)c2cccc(C(F)(F)C(C)(C)O)c2F)c2cc3c(cc12)C(C)(C)C(=O)N3C(C)C ; Cc1nnc(N[C@H](C)c2cccc(C(F)(F)CO)c2)c2cc3c(cc12)C1(CCOCC1)C(=O)N3C1CC1]

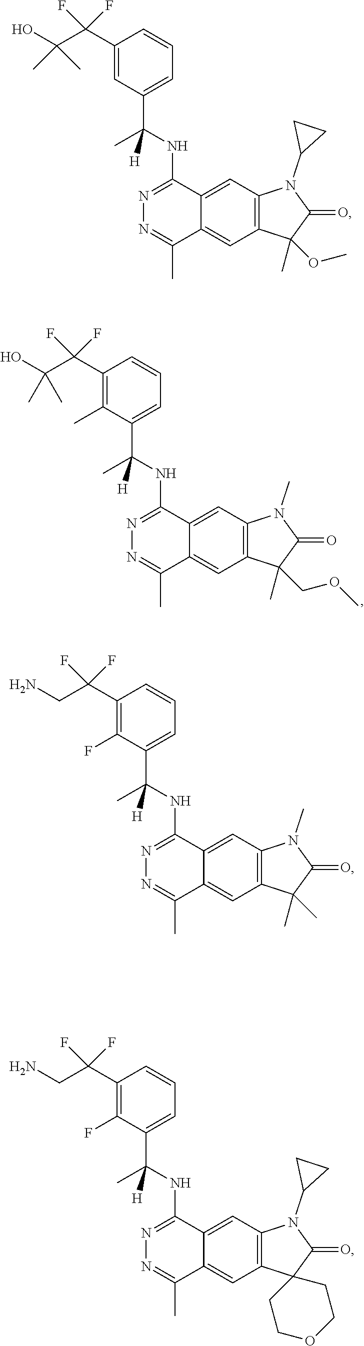 [SMILES: COC1(C)C(=O)N(C2CC2)c2cc3c(N[C@H](C)c4cccc(C(F)(F)C(C)(C)O)c4)nnc(C)c3cc21 ; COCC1(C)C(=O)N(C)c2cc3c(N[C@H](C)c4cccc(C(F)(F)C(C)(C)O)c4C)nnc(C)c3cc21 ; Cc1nnc(N[C@H](C)c2cccc(C(F)(F)CN)c2F)c2cc3c(cc12)C(C)(C)C(=O)N3C ; Cc1nnc(N[C@H](C)c2cccc(C(F)(F)CN)c2F)c2cc3c(cc12)C1(CCOCC1)C(=O)N3C1CC1]

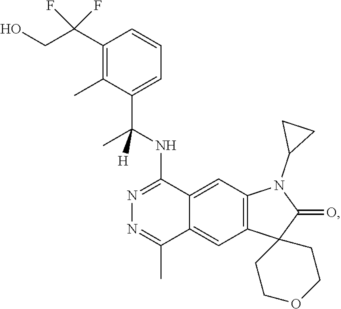 [SMILES: Cc1c([C@@H](C)Nc2nnc(C)c3cc4c(cc23)N(C2CC2)C(=O)C42CCOCC2)cccc1C(F)(F)CO]

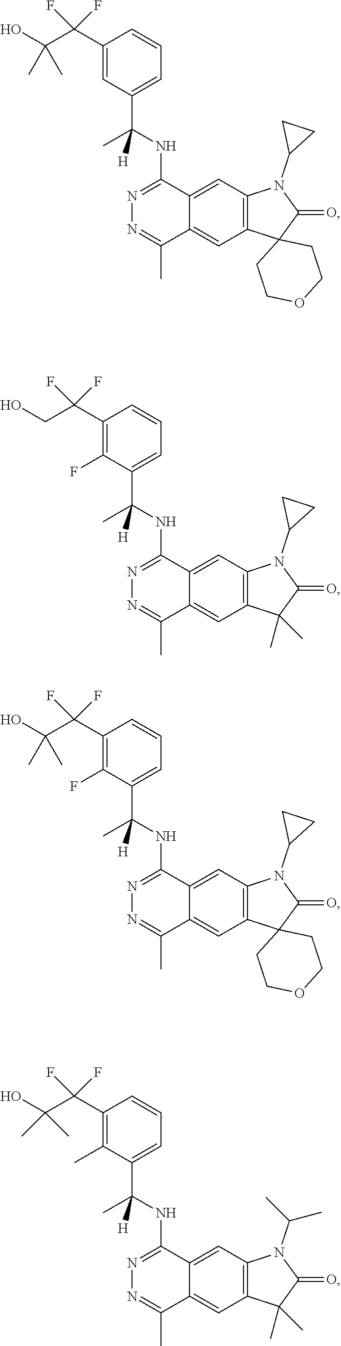 [SMILES: Cc1nnc(N[C@H](C)c2cccc(C(F)(F)C(C)(C)O)c2)c2cc3c(cc12)C1(CCOCC1)C(=O)N3C1CC1 ; Cc1nnc(N[C@H](C)c2cccc(C(F)(F)CO)c2F)c2cc3c(cc12)C(C)(C)C(=O)N3C1CC1 ; Cc1nnc(N[C@H](C)c2cccc(C(F)(F)C(C)(C)O)c2F)c2cc3c(cc12)C1(CCOCC1)C(=O)N3C1CC1 ; Cc1c([C@@H](C)Nc2nnc(C)c3cc4c(cc23)N(C(C)C)C(=O)C4(C)C)cccc1C(F)(F)C(C)(C)O]

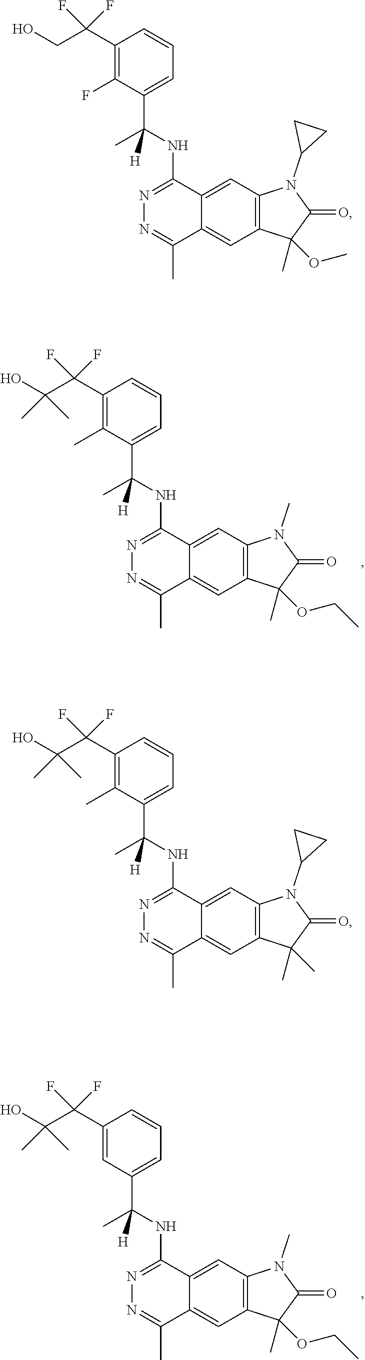 [SMILES: COC1(C)C(=O)N(C2CC2)c2cc3c(N[C@H](C)c4cccc(C(F)(F)CO)c4F)nnc(C)c3cc21 ; CCOC1(C)C(=O)N(C)c2cc3c(N[C@H](C)c4cccc(C(F)(F)C(C)(C)O)c4C)nnc(C)c3cc21 ; "," ; Cc1c([C@@H](C)Nc2nnc(C)c3cc4c(cc23)N(C2CC2)C(=O)C4(C)C)cccc1C(F)(F)C(C)(C)O ; CCOC1(C)C(=O)N(C)c2cc3c(N[C@H](C)c4cccc(C(F)(F)C(C)(C)O)c4)nnc(C)c3cc21 ; ","]

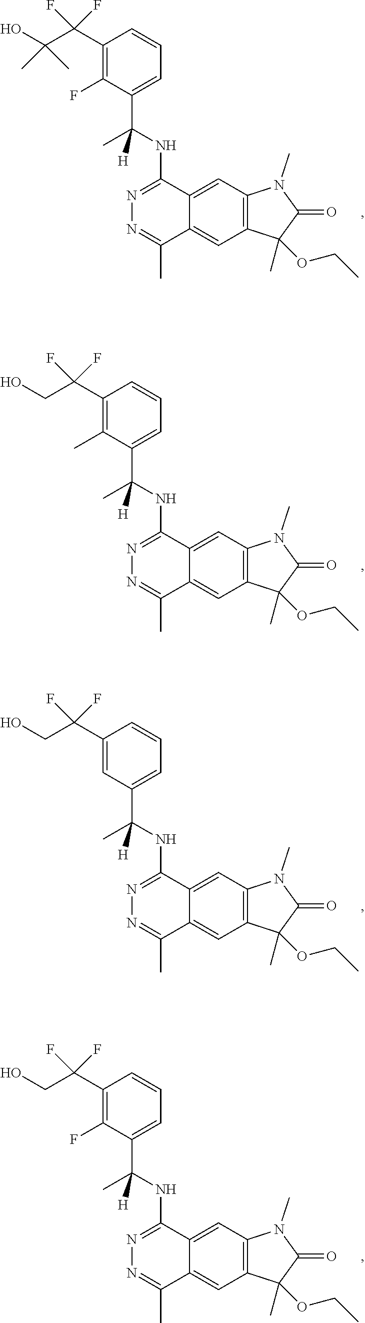

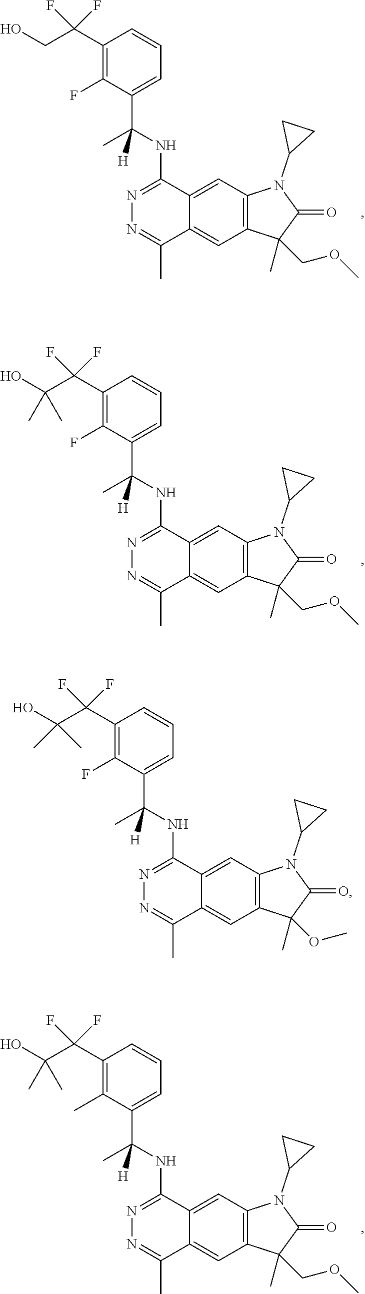

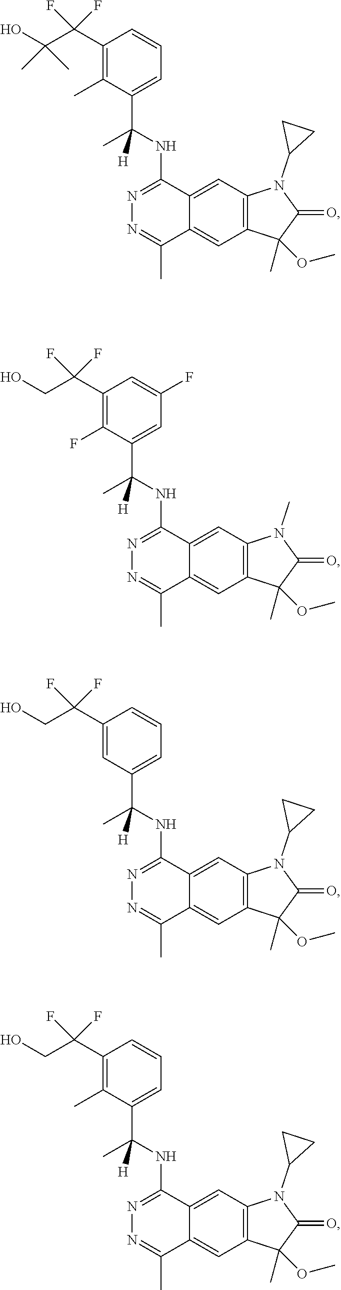 [SMILES: COC1(C)C(=O)N(C2CC2)c2cc3c(N[C@H](C)c4cccc(C(F)(F)C(C)(C)O)c4C)nnc(C)c3cc21 ; COC1(C)C(=O)N(C)c2cc3c(N[C@H](C)c4cc(F)cc(C(F)(F)CO)c4F)nnc(C)c3cc21 ; COC1(C)C(=O)N(C2CC2)c2cc3c(N[C@H](C)c4cccc(C(F)(F)CO)c4)nnc(C)c3cc21 ; COC1(C)C(=O)N(C2CC2)c2cc3c(N[C@H](C)c4cccc(C(F)(F)CO)c4C)nnc(C)c3cc21]

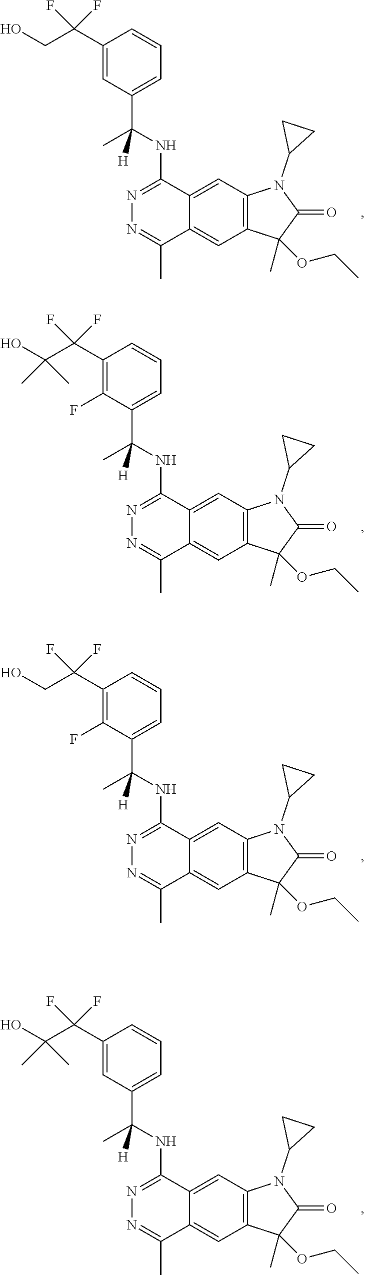

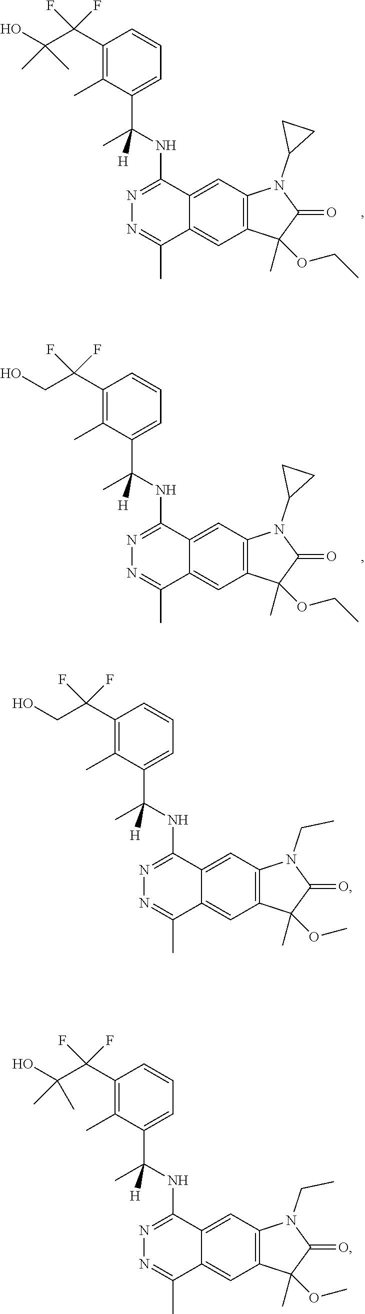 [SMILES: CCOC1(C)C(=O)N(C2CC2)c2cc3c(N[C@H](C)c4cccc(C(F)(F)C(C)(C)O)c4C)nnc(C)c3cc21 ; "," ; CCOC1(C)C(=O)N(C2CC2)c2cc3c(N[C@H](C)c4cccc(C(F)(F)CO)c4C)nnc(C)c3cc21 ; "," ; CCN1C(=O)C(C)(OC)c2cc3c(C)nnc(N[C@H](C)c4cccc(C(F)(F)CO)c4C)c3cc21 ; CCN1C(=O)C(C)(OC)c2cc3c(C)nnc(N[C@H](C)c4cccc(C(F)(F)C(C)(C)O)c4C)c3cc21]

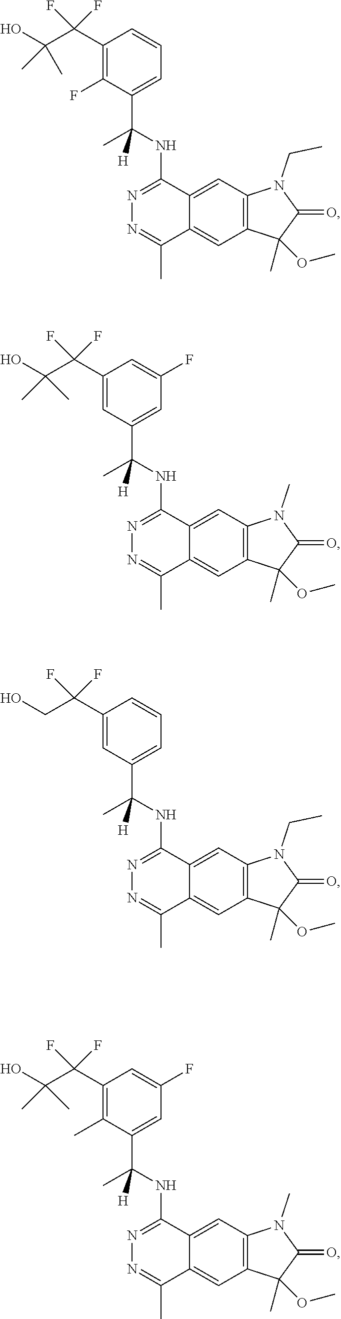 [SMILES: CCN1C(=O)C(C)(OC)c2cc3c(C)nnc(N[C@H](C)c4cccc(C(F)(F)C(C)(C)O)c4F)c3cc21 ; COC1(C)C(=O)N(C)c2cc3c(N[C@H](C)c4cc(F)cc(C(F)(F)C(C)(C)O)c4)nnc(C)c3cc21 ; CCN1C(=O)C(C)(OC)c2cc3c(C)nnc(N[C@H](C)c4cccc(C(F)(F)CO)c4)c3cc21 ; COC1(C)C(=O)N(C)c2cc3c(N[C@H](C)c4cc(F)cc(C(F)(F)C(C)(C)O)c4C)nnc(C)c3cc21]

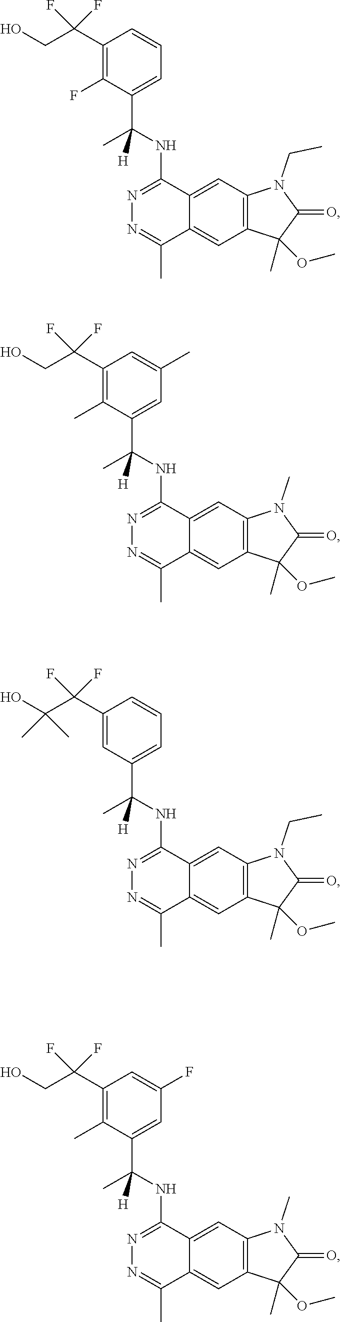 [SMILES: CCN1C(=O)C(C)(OC)c2cc3c(C)nnc(N[C@H](C)c4cccc(C(F)(F)CO)c4F)c3cc21 ; COC1(C)C(=O)N(C)c2cc3c(N[C@H](C)c4cc(C)cc(C(F)(F)CO)c4C)nnc(C)c3cc21 ; CCN1C(=O)C(C)(OC)c2cc3c(C)nnc(N[C@H](C)c4cccc(C(F)(F)C(C)(C)O)c4)c3cc21 ; COC1(C)C(=O)N(C)c2cc3c(N[C@H](C)c4cc(F)cc(C(F)(F)CO)c4C)nnc(C)c3cc21]

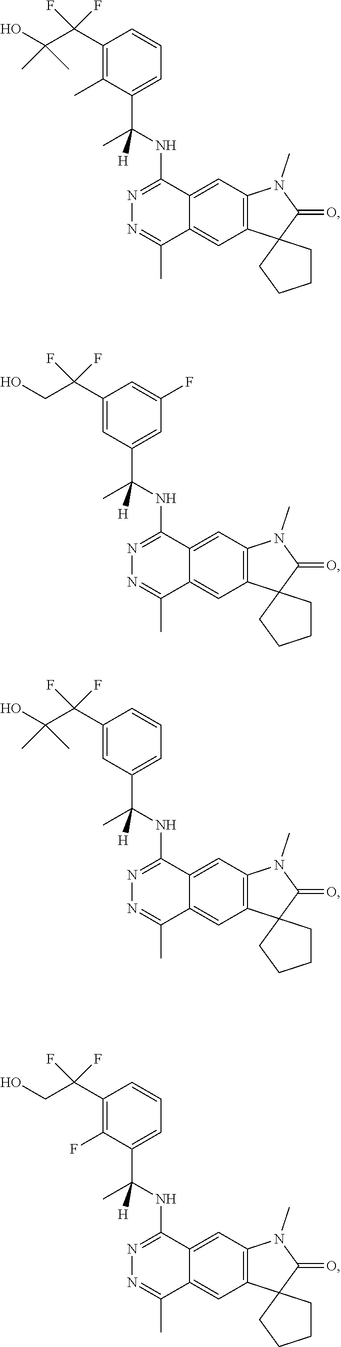 [SMILES: Cc1c([C@@H](C)Nc2nnc(C)c3cc4c(cc23)N(C)C(=O)C42CCCC2)cccc1C(F)(F)C(C)(C)O ; Cc1nnc(N[C@H](C)c2cc(F)cc(C(F)(F)CO)c2)c2cc3c(cc12)C1(CCCC1)C(=O)N3C ; Cc1nnc(N[C@H](C)c2cccc(C(F)(F)C(C)(C)O)c2)c2cc3c(cc12)C1(CCCC1)C(=O)N3C ; Cc1nnc(N[C@H](C)c2cccc(C(F)(F)CO)c2F)c2cc3c(cc12)C1(CCCC1)C(=O)N3C]

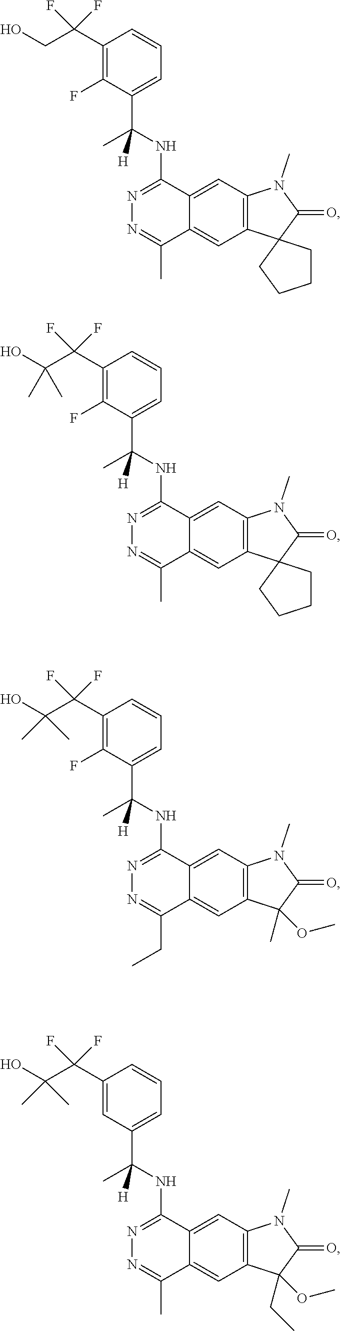 [SMILES: Cc1nnc(N[C@H](C)c2cccc(C(F)(F)CO)c2F)c2cc3c(cc12)C1(CCCC1)C(=O)N3C ; Cc1nnc(N[C@H](C)c2cccc(C(F)(F)C(C)(C)O)c2F)c2cc3c(cc12)C1(CCCC1)C(=O)N3C ; CCc1nnc(N[C@H](C)c2cccc(C(F)(F)C(C)(C)O)c2F)c2cc3c(cc12)C(C)(OC)C(=O)N3C ; CCC1(OC)C(=O)N(C)c2cc3c(N[C@H](C)c4cccc(C(F)(F)C(C)(C)O)c4)nnc(C)c3cc21]

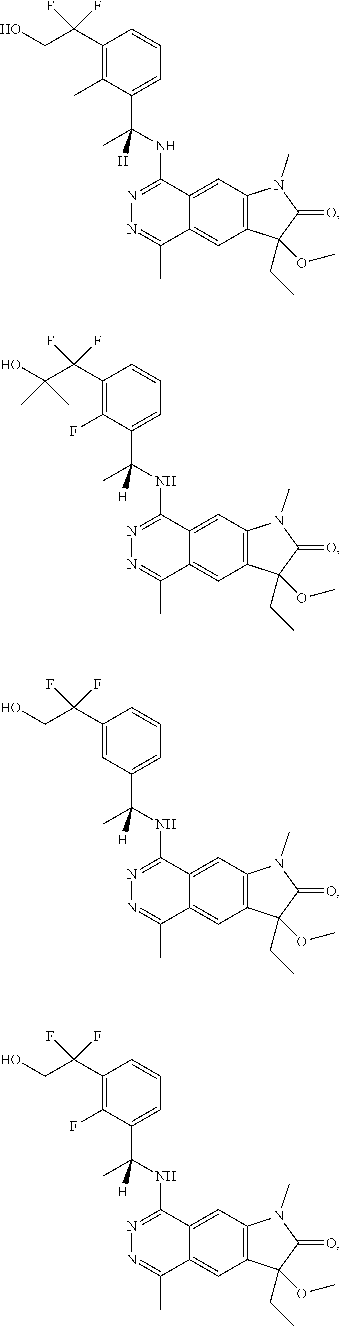 [SMILES: CCC1(OC)C(=O)N(C)c2cc3c(N[C@H](C)c4cccc(C(F)(F)CO)c4C)nnc(C)c3cc21 ; CCC1(OC)C(=O)N(C)c2cc3c(N[C@H](C)c4cccc(C(F)(F)C(C)(C)O)c4F)nnc(C)c3cc21 ; CCC1(OC)C(=O)N(C)c2cc3c(N[C@H](C)c4cccc(C(F)(F)CO)c4)nnc(C)c3cc21 ; CCC1(OC)C(=O)N(C)c2cc3c(N[C@H](C)c4cccc(C(F)(F)CO)c4F)nnc(C)c3cc21]

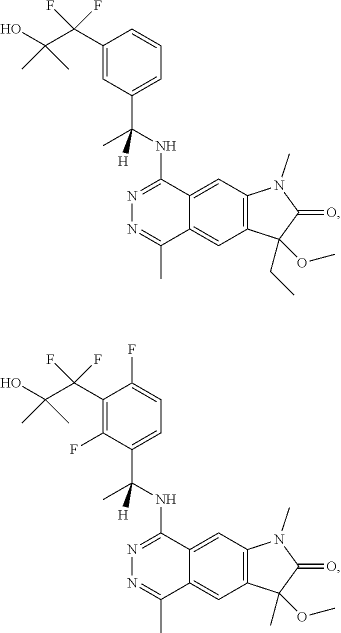 [SMILES: CCC1(OC)C(=O)N(C)c2cc3c(N[C@H](C)c4cccc(C(F)(F)C(C)(C)O)c4)nnc(C)c3cc21 ; COC1(C)C(=O)N(C)c2cc3c(N[C@H](C)c4ccc(F)c(C(F)(F)C(C)(C)O)c4F)nnc(C)c3cc21]

or a pharmaceutically acceptable salt thereof.

In some embodiments, the compound of the present disclosure is:

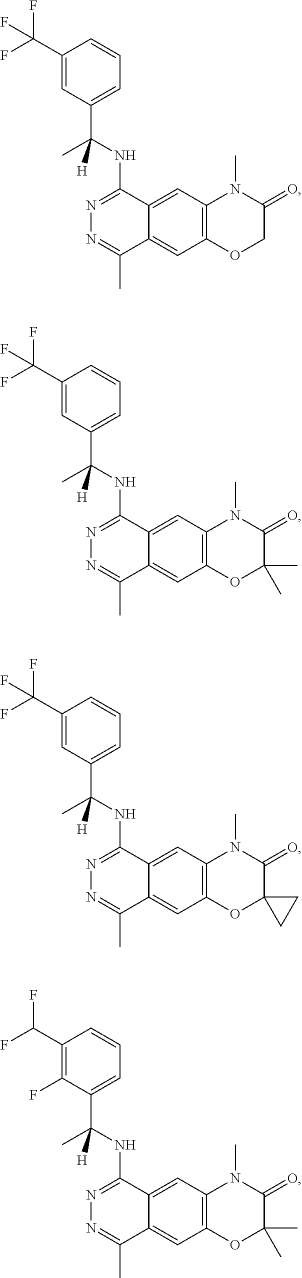 [SMILES: Cc1nnc(N[C@H](C)c2cccc(C(F)(F)F)c2)c2cc3c(cc12)OCC(=O)N3C ; Cc1nnc(N[C@H](C)c2cccc(C(F)(F)F)c2)c2cc3c(cc12)OC(C)(C)C(=O)N3C ; Cc1nnc(N[C@H](C)c2cccc(C(F)(F)F)c2)c2cc3c(cc12)OC1(CC1)C(=O)N3C ; Cc1nnc(N[C@H](C)c2cccc(C(F)F)c2F)c2cc3c(cc12)OC(C)(C)C(=O)N3C]

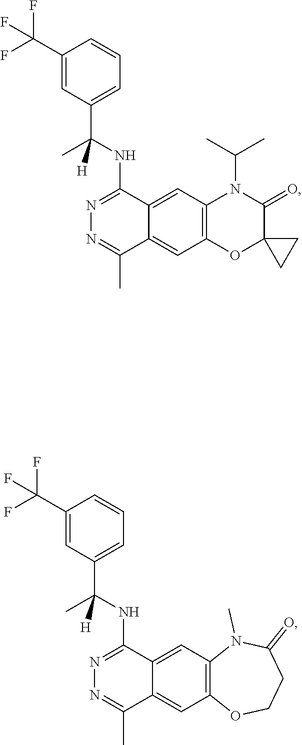 [SMILES: Cc1nnc(N[C@H](C)c2cccc(C(F)(F)F)c2)c2cc3c(cc12)OC1(CC1)C(=O)N3C(C)C ; Cc1nnc(N[C@H](C)c2cccc(C(F)(F)F)c2)c2cc3c(cc12)OCCC(=O)N3C]

or a pharmaceutically acceptable salt thereof.

In some embodiments, the compound of the present disclosure is:

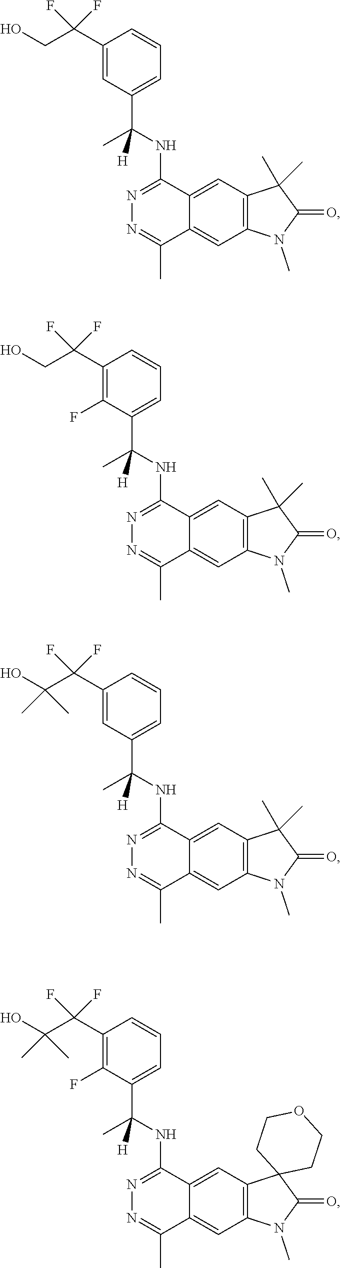 [SMILES: Cc1nnc(N[C@H](C)c2cccc(C(F)(F)CO)c2)c2cc3c(cc12)N(C)C(=O)C3(C)C ; Cc1nnc(N[C@H](C)c2cccc(C(F)(F)CO)c2F)c2cc3c(cc12)N(C)C(=O)C3(C)C ; Cc1nnc(N[C@H](C)c2cccc(C(F)(F)C(C)(C)O)c2)c2cc3c(cc12)N(C)C(=O)C3(C)C ; Cc1nnc(N[C@H](C)c2cccc(C(F)(F)C(C)(C)O)c2F)c2cc3c(cc12)N(C)C(=O)C31CCOCC1]

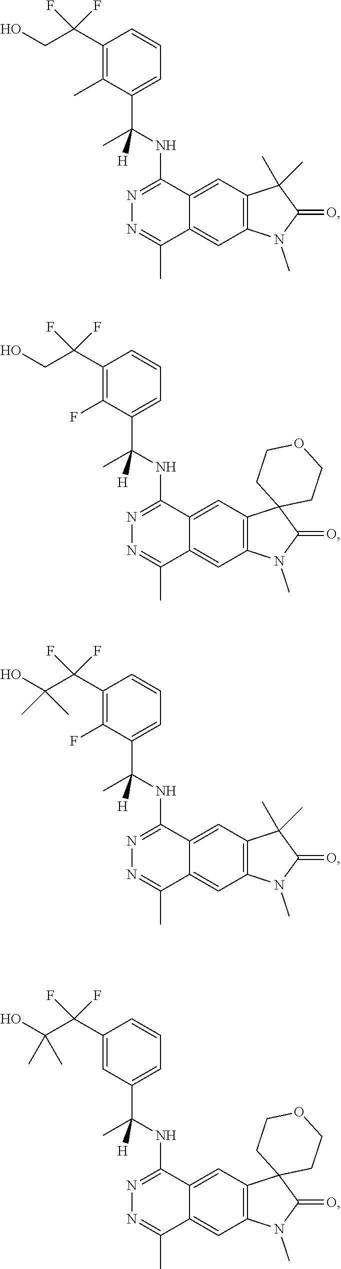 [SMILES: Cc1c([C@@H](C)Nc2nnc(C)c3cc4c(cc23)C(C)(C)C(=O)N4C)cccc1C(F)(F)CO ; Cc1nnc(N[C@H](C)c2cccc(C(F)(F)CO)c2F)c2cc3c(cc12)N(C)C(=O)C31CCOCC1 ; Cc1nnc(N[C@H](C)c2cccc(C(F)(F)C(C)(C)O)c2F)c2cc3c(cc12)N(C)C(=O)C3(C)C ; Cc1nnc(N[C@H](C)c2cccc(C(F)(F)C(C)(C)O)c2)c2cc3c(cc12)N(C)C(=O)C31CCOCC1]

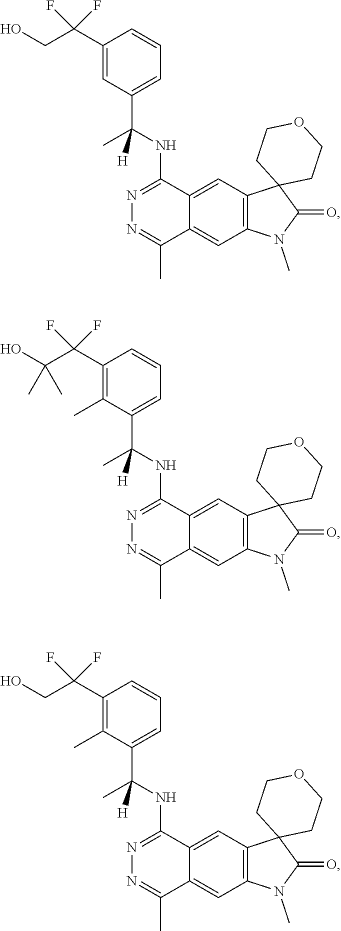 [SMILES: Cc1nnc(N[C@H](C)c2cccc(C(F)(F)CO)c2)c2cc3c(cc12)N(C)C(=O)C31CCOCC1 ; Cc1c([C@@H](C)Nc2nnc(C)c3cc4c(cc23)C2(CCOCC2)C(=O)N4C)cccc1C(F)(F)C(C)(C)O ; Cc1c([C@@H](C)Nc2nnc(C)c3cc4c(cc23)C2(CCOCC2)C(=O)N4C)cccc1C(F)(F)CO]

or a pharmaceutically acceptable salt thereof.

In some embodiments, the compound of the present disclosure is a compound provided in Table 4A, 4B, 4C, or 4D, or a pharmaceutically acceptable salt thereof.

In some embodiments, the compound of the present disclosure is a compound provided in Table 5, or a pharmaceutically acceptable salt thereof.

### Pharmaceutical Compositions

In various embodiments, the present disclosure provides a pharmaceutical composition comprising a compound disclosed herein (e.g., a compound of Formula (I), Formula (Ia), Formula (Ia-1), Formula (Ib), Formula (Ib-1), Formula (Ic), or Formula (Ic-1)) or a pharmaceutically acceptable salt thereof, and a pharmaceutically acceptable excipient.

In various embodiments, the present disclosure provides a pharmaceutical composition comprising a therapeutically effective amount of a compound disclosed herein (e.g., a compound of Formula (I), Formula (Ia), Formula (Ia-1), Formula (Ib), Formula (Ib-1), Formula (Ic), or Formula (Ic-1)) or a pharmaceutically acceptable salt thereof, and a pharmaceutically acceptable excipient.

In some embodiments, the pharmaceutically acceptable salt is a salt of 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid, (−L) malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, propionic acid, pyroglutamic acid (−L), salicylic acid, sebacic acid, stearic acid, succinic acid, sulfuric acid, tartaric acid (+L), thiocyanic acid, toluenesulfonic acid (p), and undecylenic acid.

The pharmaceutically acceptable excipients and adjuvants are added to the composition or formulation for a variety of purposes. In some embodiments, a pharmaceutical composition comprising one or more compounds disclosed herein, or a pharmaceutically acceptable salt thereof, further comprise a pharmaceutically acceptable carrier. In some embodiments, a pharmaceutically acceptable carrier includes a pharmaceutically acceptable excipient, binder, and/or diluent. In some embodiments, suitable pharmaceutically acceptable carriers include, but are not limited to, inert solid fillers or diluents and sterile aqueous or organic solutions. In some embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like. General considerations in the formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21^{st} Edition (Lippincott Williams & Wilkins, 2005).

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

### Methods of Treatment

The present disclosure is directed, in-part, to SOS1 inhibitor compounds of the present disclosure, which are useful in the treatment and/or prevention of a disease and/or condition associated with or modulated by SOS1, including wherein the inhibition of the interaction of SOS1 and a RAS-family protein and/or RAC1 is of therapeutic benefit for the treatment and/or prevention of cancer.

In some embodiments, the present disclosure provides a method of treating and/or preventing cancer comprising administering to a subject a therapeutically effective amount of a compound disclosed herein (e.g., a compound of Formula (I), Formula (Ia), Formula (Ib), Formula (Ic), or Formula (Ic-1)), a pharmaceutically acceptable salt thereof, or a pharmaceutical composition thereof.

In some embodiments, the compound of the present disclosure or pharmaceutically acceptable salt thereof is an inhibitor of SOS1.

In some embodiments, the present disclosure provides a method of treating and/or preventing a disease by inhibiting the interaction of SOS1 and a RAS-family protein or RAC1, the method comprising administering to a subject a therapeutically effective amount of a compound disclosed herein (e.g., a compound of Formula (I), Formula (Ia), Formula (Ib), Formula (Ic), or Formula (Ic-1)), a pharmaceutically acceptable salt thereof, or a pharmaceutical composition thereof.

In some embodiments, the present disclosure provides a compound disclosed herein (e.g., a compound of Formula (I), Formula (Ia), Formula (Ib), Formula (Ic), or Formula (Ic-1)), a pharmaceutically acceptable salt thereof, or a pharmaceutical composition thereof, for use in a method of treating and/or preventing a disease, such as a disease associated with or modulated by SOS1.

In some embodiments, the present disclosure provides the use of a compound disclosed herein (e.g., a compound of Formula (I), Formula (Ia), Formula (Ib), Formula (Ic), or Formula (Ic-1)), a pharmaceutically acceptable salt thereof, or a pharmaceutical composition thereof, for the manufacture of a medicament for treating a disease, such as a diseases associated with or modulated by SOS1.

In some embodiments, the disease is cancer. In some embodiments, the cancer is pancreatic cancer, lung cancer, colorectal cancer, cholangiocarcinoma, multiple myeloma, melanoma, uterine cancer, endometrial cancer, thyroid cancer, acute myeloid leukemia, bladder cancer, urothelial cancer, gastric cancer, cervical cancer, head and neck squamous cell carcinoma, diffuse large B cell lymphoma, oesophageal cancer, chronic lymphocytic leukemia, hepatocellular cancer, breast cancer, ovarian cancer, prostate cancer, glioblastoma, renal cancer or sarcoma. In some embodiments, the cancer is pancreatic cancer, lung cancer (e.g., non-small cell lung cancer (NSCLC)), cholangiocarcinoma or colorectal cancer.

In one aspect the disease/condition/cancer to be treated/prevented with a compound of the present disclosure (e.g., a SOS1 inhibitor compound) is a disease/condition/cancer defined as exhibiting one or more of the following molecular features:

### 1. Kras Alterations:

a. KRAS amplification (wild type (wt) or mutant);
b. KRAS overexpression (wt or mutant);
c. KRAS mutation(s):
   i. G12 mutations (e.g., G12C, G12V, G12S, G12A, G12V, G12R, G12F, G12D);
   ii. G13 mutations (e.g., G13C, G13D, G13R, G13V, G13S, G13A)
   iii. T35 mutation (e.g., T35I);
   iv. 136 mutation (e.g., I36L, I36M);
   v. E49 mutation (e.g., E49K);
   vi. Q61 mutation (e.g., Q61H, Q61R, Q61P, Q61E, Q61K, Q61L, Q61K);
   vii. K117 mutation (e.g., K117N);
   viii. A146 mutation (e.g., A146T, A146V);

### 2. NRAS Alterations:

a. NRAS amplification (wt or mutant);
b. NRAS overexpression (wt or mutant);
c. NRAS mutation(s):
   i. G12 mutations (e.g., G12A, G12V, G12D, G12C, G12S, G12R);
   ii. G13 mutation (e.g., G13V, G13D, G13R, G13S, G13C, G13A);
   iii. Q61 mutation (e.g., Q61K, Q61L, Q61H, Q61P, Q61R);
   iv. A146 mutation (e.g., A146T, A146V);

### 3. HRAS Alterations:

a. HRAS amplification (wt or mutant);
b. HRAS overexpression (wt or mutant);
c. HRAS mutation(s);
   i. G12 mutation (e.g., G12C, G12V, G12S, G12A, G12V, G12R, G12F, G12D);
   ii. G13 mutation (e.g., G13C, G13D, G13R, G13V, G13S, G13A);
   iii. Q61 mutation (e.g., Q61K, Q61L, Q61H, Q61P, Q61R);

### 4. EGFR Alterations:

a. EGFR amplification (wt or mutant);
b. EGFR overexpression (wt or mutant);
c. EGFR mutation(s)
   i. e.g., exon 20 insertion, exon 19 deletion (De119), G719X (e.g., G719A, G719C, G719S), T790M, C797S, T854A, L858R, L861Q, or any combination thereof;

### 5. BRAF Alterations:

a. BRAF amplifications
b. BRAF overexpression
c. BRAF mutation(s) e.g., Class 2; G464V, G469V, L597Q, K601E, or Class 3; D287H, V459L, G466V
d. Chromosomal rearrangement involving the BRAF gene

### 6. ErbB2 (Her2) Alterations:

a. ErbB2 amplification;
b. ErbB2 overexpression;
c. ErbB2 mutation(s)
   i. e.g., R678, G309, L755, D769, D769, V777, P780, V842, R896, c.2264_2278del (L755_T759del), c.2339_2340ins (G778_P780dup), S310;
      7. c-MET Alterations:
a. c-MET amplification;
b. c-MET overexpression;
c. c-MET mutation(s)
   i. e.g., E168, N375, Q648, A887, E908, T1010, V1088, H1112, R1166, R1188, Y1248, Y1253, M1268, D1304, A1357, P1382;

### 8. AXL Alterations:

a. AXL amplification;
b. AXL overexpression;

### 9. BCR-ABL Alterations:

a. chromosomal rearrangements involving the ABL gene;

### 10. ALK Alterations:

a. ALK amplification;
b. ALK overexpression;
c. ALK mutation(s)
   i. e.g., 1151Tins, L1152R, C1156Y, F1174L, L1196M, L1198F, G1202R, S1206Y, G1269A;
d. chromosomal rearrangements involving the ALK gene;

### 11. FGFR1 Alterations:

a. FGFR1 amplification;
b. FGFR1 overexpression;

### 12. FGFR2 Alterations:

a. FGFR2 amplification;
b. FGFR2 overexpression;

### 13. FGFR3 Alterations:

a. FGFR3 amplification;
b. FGFR3 overexpression;
c. chromosomal rearrangement involving the FGFR3 gene;

### 14. FGFR4 Alterations:

a. FGFR4 amplification
b. FGFR4 overexpression
c. FGFR4 mutations (e.g., N535K, V550L, V550M)
d. Chromosomal rearrangement involving the FGFR4 gene
   15. cKIT Alterations:
a. cKIT amplification
b. cKIT overexpression
c. cKIT mutations (e.g., exon 9 insertions, exon 11 alterations (insertion or deletion), W557R, V559D, V560D, L576P, K642E, V654A, D816V, D820Y, N822K, Y823D, A829P, R888W)

### 16. PDGFRA Alterations:

a. PDGFRA amplification
b. PDGFRA overexpression
c. PDGFRA mutations (e.g., D842V, N659Y)

### 17. NTRK1 Alterations:

a. chromosomal rearrangements involving the NTRK1 gene;

### 18. NF1 Alterations:

a. NF1 mutation(s) (e.g., R440*, I679Dfs*21, R1241*, Y2285Tfs*5, R2450*)
b. NF1 gene deletions/microdeletions

### 19. RET Alterations:

a. RET amplification;
b. RET overexpression;
c. chromosomal rearrangements involving the RET gene

### 20. ROS1 Alterations:

a. ROS1 amplification;
b. ROS1 overexpression;
c. ROS1 mutation(s)
   i. e.g., G2032R, D2033N, L2155S;
d. chromosomal rearrangements involving the ROS1 gene;

### 21. SOS1 Alterations

a. SOS1 amplification;
b. SOS1 overexpression;
c. SOS1 mutation(s);

### 22. RAC1 Alterations

a. RAC1 amplification;
b. RAC1 overexpression;
c. RAC1 mutation(s);

### 23. MDM2 Alterations

a. MDM2 amplification
b. MDM2 overexpression
c. MDM2 amplification in combination with functional p53
d. MDM2 amplification in combination with wild-type p53

### 24. RAS Wild-Type

a. KRAS wild-type
a. HRAS wild-type
b. NRAS wild-type

In some embodiments, the cancer to be treated with an SOS1 inhibitor of the present disclosure is:
(a) lung adenocarcinoma harboring a KRAS mutation selected from the group consisting of G12C, G12V, G12D and G12R;
(b) colorectal adenocarcinoma harboring a KRAS mutation selected from the group consisting of G12D, G12V, G12C, G12R and G13D; or
(c) pancreatic adenocarcinoma harboring a KRAS mutation selected from the group consisting of G12D, G12V, G12R, G12C and Q61 H.

In some embodiments, the disease/condition to be treated/prevented with the SOS1 inhibitor compound of the present disclosure is a RASopathy. In some embodiments, the disease/condition is Neurofibromatosis type 1 (NF1), Noonan Syndrome (NS), Noonan Syndrome with Multiple Lentigines (NSML) (also referred to as LEOPARD syndrome), Capillary Malformation-Arteriovenous Malformation Syndrome (CM-AVM), Costello Syndrome (CS), Cardio-Facio-Cutaneous Syndrome (CFC), Legius Syndrome (also known as NF1-like Syndrome), or Hereditary gingival fibromatosis.

In some embodiments, the present methods comprise administering to the subject in need thereof a compound of Formula (Ia-1):

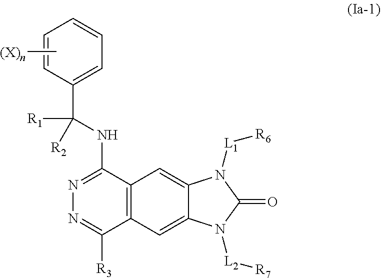

or a pharmaceutically acceptable salt thereof, wherein X, R₁, R₂, R₃, R₆, R₇, L₁, L₂, and n are as defined herein.

In some embodiments, the present methods comprise administering to the subject in need thereof a compound of Formula (Ib-1):

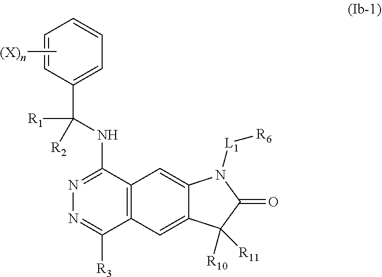

or a pharmaceutically acceptable salt thereof, wherein X, R₁, R₂, R₃, R₆, R₁₀, R₁₁, L₁, and n are as defined herein.

In some embodiments, the present methods comprise administering to the subject in need thereof a compound of Formula (Ic-1):

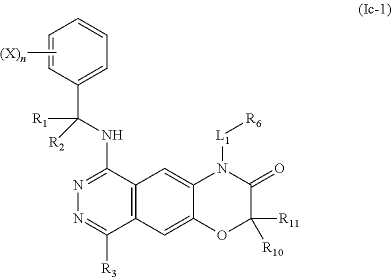

or a pharmaceutically acceptable salt thereof, wherein X, R₁, R₂, R₃, R₆, R₁₀, R₁₁, L₁, and n are as defined herein.

In some embodiments, the present methods comprise administering to the subject in need thereof a compound of Formula (Id-1):

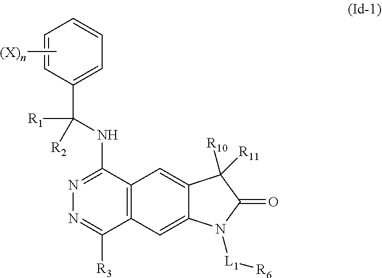

or a pharmaceutically acceptable salt thereof, wherein X, R₁, R₂, R₃, R₆, R₁₀, R₁₁, L₁, and n are as defined herein.

### EXAMPLES

### Materials and Methods

Solvents, reagents and starting materials were purchased from commercial vendors and used as received unless otherwise described. All reactions were performed at room temperature unless otherwise stated. Compound identity and purity confirmations were performed by LCMS UV using a Waters Acquity SQ Detector 2 (ACQ-SQD2 #LCA081). The diode array detector wavelength was 254 nM and the MS was in positive and negative electrospray mode (m/z: 150-800). A 2 μL aliquot was injected onto a guard column (0.2 μm×2 mm filters) and UPLC column (C18, 50×2.1 mm, <2 μm) in sequence maintained at 40° C. The samples were eluted at a flow rate of 0.6 mL/min with a mobile phase system composed of A (0.1% (v/v) Formic Acid in Water) and B (0.1% (v/v) Formic Acid in Acetonitrile) according to the gradients outlined in Table 1 below. Retention times (RT) are reported in minutes.

TABLE 1A
LCMS solvent gradients for compound analysis.

| | | |
|---|---|---|
| Time (min) | % A | % B |

Method 1 (Long acidic)

| | | |
|---|---|---|
| 0 | 95 | 5 |
| 1.1 | 95 | 5 |
| 6.1 | 5 | 95 |
| 7 | 5 | 95 |
| 7.5 | 95 | 5 |
| 8 | 95 | 5 |

Method 2 (Short acidic)

| | | |
|---|---|---|
| 0 | 95 | 5 |
| 0.3 | 95 | 5 |
| 2 | 5 | 95 |
| 2.6 | 95 | 5 |
| 3 | 95 | 5 |

NMR was also used to characterize final compounds. NMR spectra were obtained on a Bruker AVIII 400 Nanobay with 5 mm BBFO probe. Optionally, compound R_{f} values on silica thin layer chromatography (TLC) plates were measured.

Compound purification was performed by flash column chromatography on silica or by preparative LCMS. LCMS purification was performed using a Waters 3100 Mass detector in positive and negative electrospray mode (m/z: 150-800) with a Waters 2489 UV/Vis detector.

Samples were eluted at a flow rate of 20 mL/min on a XBridge™ prep C18 5 μM OBD 19×100 mm column with a mobile phase system composed of A (0.1% (v/v) Formic Acid in Water) and B (0.1% (v/v) Formic Acid in Acetonitrile) according to the gradient outlined in Table 2 below.

TABLE 2
LCMS solvent gradients for compound purification.

| Time (min) | % A | % B |
|---|---|---|
| 0 | 90 | 10 |
| 1.5 | 90 | 10 |
| 11.7 | 5 | 95 |
| 13.7 | 5 | 95 |
| 14 | 90 | 90 |
| 15 | 90 | 90 |

### Example 1. Synthesis of Intermediates

### 5-chloro-3-cyclopentyl-1,8-dimethyl-imidazo[4,5-g]phthalazin-2-one (Int-1)

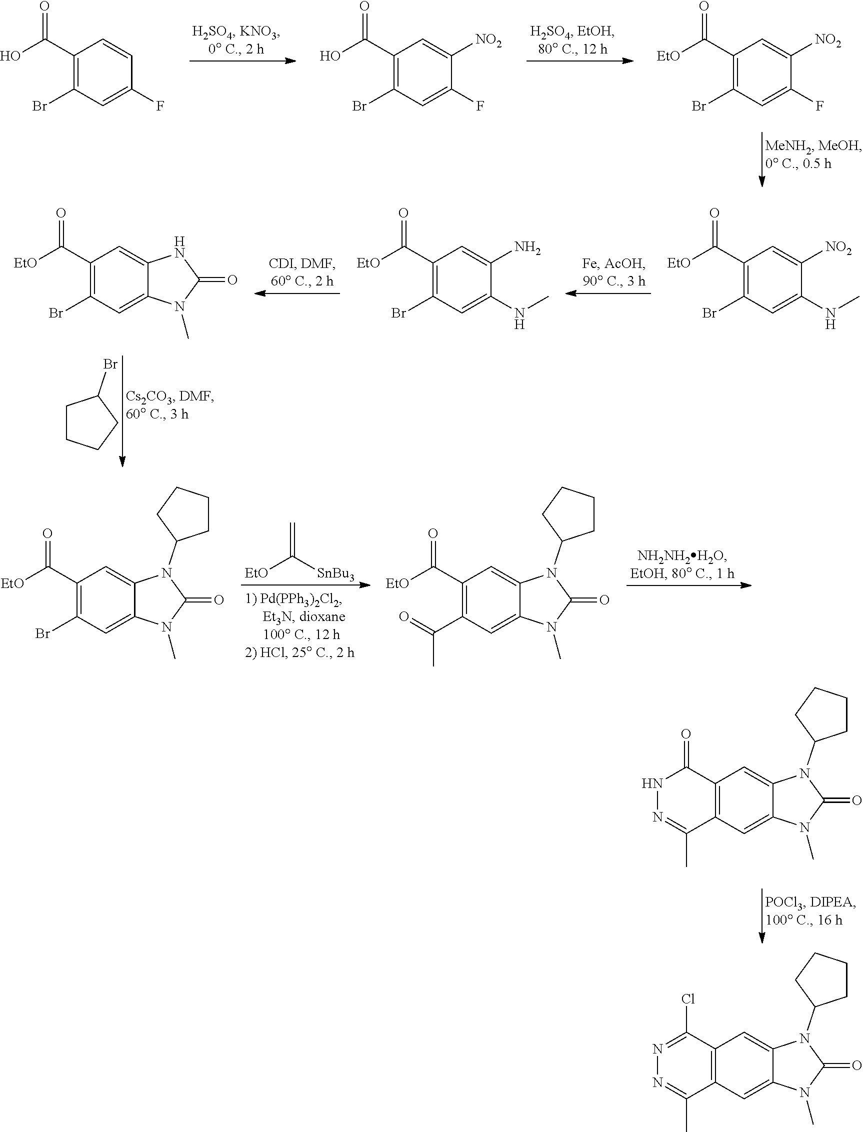

### Step 1

2-Bromo-4-fluorobenzoic acid (3.g, 13.7 mmol) was suspended in sulfuric acid (26.3 mL, 493 mmol) and cooled to 0° C. Potassium nitrate (1.45 g, 14.4 mmol) was then added in portions and the reaction stirred for 2 hours. Ice-cooled water was then added to the reaction mixture. The resulting precipitate was then filtered under vacuum and dried. This gave 2-bromo-4-fluoro-5-nitro-benzoic acid (3.52 g, 13.3 mmol, 97.3% yield) as a white solid.

¹H NMR (400 MHz, DMSO-d₆): δ 8.50 (d, J=8.1 Hz, 1H), 8.18 (d, J=10.9 Hz, 1H)

### Step 2

To a stirring solution of 2-bromo-4-fluoro-5-nitro-benzoic acid (3.52 g, 13.3 mmol) in ethanol (28.1 mL) was added sulfuric acid (1.42 mL, 26.7 mmol). The solution was heated at reflux overnight. More sulfuric acid (1.42 mL, 26.7 mmol) was added. After 3 more hours, all volatiles were removed under reduced pressure. The residue was carefully quenched with ice-cold NaHCO₃. The aqueous phase was extracted with ethyl acetate (×3). The combined organic phases were dried over MgSO₄ and concentrated to give ethyl 2-bromo-4-fluoro-5-nitro-benzoate (2.4 g, 8.22 mmol, 61.6% yield), a green solid.

UPLC-MS (ES⁺, Short acidic): 1.82 min, [M+H]⁺ (81%), mass not observed

¹H NMR (400 MHz, DMSO-d₆): δ 8.52 (d, J=8.1 Hz, 1H), 8.21 (d, J=10.6 Hz, 1H), 4.37 (q, J=7.1 Hz, 2H), 1.35 (t, J=7.2 Hz, 3H)

### Step 3

To a stirring suspension of ethyl 2-bromo-4-fluoro-5-nitrobenzoate (2.25 g, 7.70 mmol) in methanol (11.3 mL) at 0° C. was added methylamine (2M in THF, 7.7 mL, 15.4 mmol) dropwise, over the course of around 10 minutes (suspension becomes very thick over course of addition). After 30 minutes at 0° C., diethyl ether (30 mL) was added, and the suspension filtered. The solid was washed with more diethyl ether and dried under vacuum to yield ethyl 2-bromo-4-(methylamino)-5-nitrobenzoate (1.43 g, 4.72 mmol, 61.2% yield) as an orange solid.

UPLC-MS (ES⁺, Short acidic): 1.83 min, m/z 305.0 [M+H]⁺ (100%)

¹H NMR (400 MHz, DMSO-d₆): δ 8.59 (s, 1H), 8.58-8.54 (br s, 1H), 7.29 (s, 1H), 4.29 (q, J=7.2 Hz, 2H), 3.00 (d, J=5.0 Hz, 3H), 1.33 (t, J=7.2 Hz, 3H)

### Step 4

To a stirring suspension of ethyl 2-bromo-4-(methylamino)-5-nitro-benzoate (1.4 g, 4.62 mmol) in acetic acid (23.1 mL) was added iron powder (0.77 g, 13.9 mmol). The resulting mixture was heated to 90° C. for 3 hours. All insoluble were then removed by filtration, and the filtrate was neutralised with 1M aq. potassium carbonate, while cooling. The aqueous solution was then extracted with ethyl acetate (3×). The combined organic phases were dried over MgSO4, filtered and concentrated. The residue was purified by flash chromatography (25 g cartridge, eluent ethyl acetate in petroleum ether 0-50%) yielded ethyl 5-amino-2-bromo-4-(methylamino)benzoate (421 mg, 1.54 mmol, 33.4% yield) as a brown solid.

UPLC-MS (ES⁺, Short acidic): 1.55 min, m/z 275.0 [M+H]⁺ (100%)

¹H NMR (400 MHz, DMSO-d₆): δ 7.13 (s, 1H), 6.51 (s, 1H), 5.59-5.57 (m, 1H), 4.86 (br s, 2H), 4.20 (q, J=7.1 Hz, 2H), 4.76 (d, J=4.8 Hz, 3H), 1.28 (t, J=7.1 Hz, 3H)

### Step 5

In a microwave vial, 1,1′-carbonyldiimidazole (475 mg, 2.93 mmol) was added to a stirring solution of ethyl 5-amino-2-bromo-4-(methylamino)benzoate (400.mg, 1.46 mmol) in DMF (2.5 mL). The tube was then sealed and heated to 60° C. After 2 hours, the reaction mixture was cooled to room temperature. Ice-cold water was added, and the resulting precipitate filtered under vacuum and air-dried to give ethyl 6-bromo-1-methyl-2-oxo-3H-benzimidazole-5-carboxylate (403 mg, 1.35 mmol, 91.9% yield) as a brown solid.

UPLC-MS (ES⁺, Short acidic): 1.47 min, m/z 301.0 [M+H]⁺ (100%)

¹H NMR (400 MHz, DMSO-d₆): δ 11.20 (s, 1H), 7.51 (s, 1H), 7.39 (s, 1H), 4.29 (q, J=7.2 Hz, 2H), 3.31 (s, 3H), 1.32 (t, J=7.1 Hz, 3H)

### Step 6

To a vigorously stirring suspension of ethyl 6-bromo-1-methyl-2-oxo-3H-benzimidazole-5-carboxylate (390.mg, 1.3 mmol), cesium carbonate (1.27 g, 3.91 mmol) in DMF (10 mL), was added bromocyclopentane (210 μL, 1.96 mmol). After stirring for 24 hours at room temperature, more bromocyclopentane (210 μL, 1.96 mmol) was added and warmed to 60° C. After 3 hours, the suspension was cooled to room temperature then water and ethyl acetate were added. The aqueous phase was extracted with ethyl acetate (3×). The combined organic phases were washed with brine, passed through hydrophobic filter paper and concentrated to yield ethyl 6-bromo-3-cyclopentyl-1-methyl-2-oxo-benzimidazole-5-carboxylate (461 mg, 1.25 mmol, 96.3% yield) as a brown solid.

UPLC-MS (ES⁺, Short acidic): 1.89 min, m/z 369.1 [M+H]⁺ (60%)

¹H NMR (400 MHz, CDCl₃): δ 7.55 (s, 1H), 7.23 s(s, 1H), 4.81 (p, J=8.9 Hz, 1H), 4.41 (q, J=7.2 Hz, 2H), 3.39 (s, 3H), 2.08-2.02 (m, 8H), 1.43 (t, J=7.2 Hz, 3H)

### Step 7

To a stirring, thoroughly degassed solution of ethyl 6-bromo-3-cyclopentyl-1-methyl-2-oxo-benzimidazole-5-carboxylate (409.mg, 1.11 mmol), tributyl(1-ethoxyvinyl)tin (452 uL, 1.34 mmol) and triethylamine (388 uL, 2.78 mmol) in 1,4-Dioxane (8 mL), was added bis(triphenylphosphine)palladium(II) dichloride (78.2 mg, 0.11 mmol). The reaction vessel was then sealed and heated to 100° C. overnight. The reaction mixture was then cooled to room temperature and hydrogen chloride (2.78 mL, 5.57 mmol) was added. After stirring for 2 hours, water and ethyl acetate were added, and the layers were separated. The aqueous phase was extracted with ethyl acetate (3×). The combined organic phases were washed with brine, passed through hydrophobic filter paper and concentrated. The crude was purified by flash chromatography (12 g column, eluent ethyl acetate in petroleum ether 0-100%) to yield ethyl 6-acetyl-3-cyclopentyl-1-methyl-2-oxo-benzimidazole-5-carboxylate (195 mg, 0.59 mmol, 53.0% yield) as a brown solid.

UPLC-MS (ES⁺, Short acidic): 1.69 min, m/z 353.2 [M+Na]⁺ (100%)

¹H NMR (400 MHz, CDCl₃): δ 7.54 (s, 1H), 6.96 (s, 1H), 4.89-4.78 (m, 1H), 4.37 (q, J=7.2 Hz, 2H), 3.44 (s, 3H), 2.54 (s, 3H), 2.14-1.95 (m, 6H), 1.79-1.69 (m, 2H), 1.39 (t, J=7.2 Hz, 3H)

### Step 8

To a stirring suspension of ethyl 6-acetyl-3-cyclopentyl-1-methyl-2-oxo-benzimidazole-5-carboxylate (180.mg, 0.54 mmol) in ethanol (3.5 mL), was added hydrazine hydrate (72.uL, 0.82 mmol). The reaction mixture was heated to 80° C. After 1 hour, all volatiles were removed to yield 1-cyclopentyl-3,5-dimethyl-7H-imidazo[4,5-g]phthalazine-2,8-dione (158 mg, 0.53 mmol, 97.2% yield) as a grey solid.

UPLC-MS (ES⁺, Short acidic): 1.35 min, m/z 299.2 [M+H]⁺ (98%).

¹H NMR (400 MHz, DMSO-d₆): δ 12.28 (s, 1H), 7.90 (s, 1H), 7.61 (s, 1H), 4.95-4.68 (m, 1H), 3.48 (s, 3H), 2.56 (s, 3H), 2.13-1.86 (m, 8H)

### Step 9

1-Cyclopentyl-3,5-dimethyl-7H-imidazo[4,5-g]phthalazine-2,8-dione (97.3 mg, 0.33 mmol) was suspended in phosphorus oxychloride (1.5 mL, 16.1 mmol) and N,N-diisopropylethylamine (284 uL, 1.63 mmol) was added. The reaction mixture was heated to 100° C. for 2 hours, after which time excess phosphorus oxychloride was removed under reduced pressure to afford 5-chloro-3-cyclopentyl-1,8-dimethyl-imidazo[4,5-g]phthalazin-2-one (102 mg, 0.32 mmol, 98.7% yield) as a green oil, which was used directly in the next step.

UPLC-MS (ES⁺, Short acidic): 1.56 min, m/z 317.0 [M+H]⁺ (100%)

The following chlorophthalazines were prepared in a similar manner. Example 7 was made using 2-methoxyethylamine instead of methylamine.

TABLE 3A
Intermediates Prepared According to the Disclosed Methods

| Compound | Structure | Analytical data |
|---|---|---|
| Int-2 | 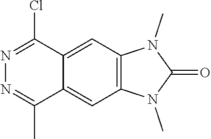 [SMILES: Cc1nnc(Cl)c2cc3c(cc12)n(C)c(=O)n3C] | UPLC-MS (ES⁺, Short acidic): 1.13 min, m/z 263.1 [M + H]⁺ (66%) |
| Int-3 | 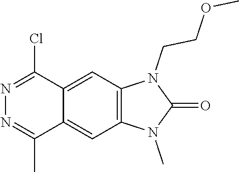 [SMILES: COCCn1c(=O)n(C)c2cc3c(C)nnc(Cl)c3cc21] | UPLC-MS (ES+, Short acidic): 1.15 min, m/z 307.5 [M + H]⁺ (97%) |
| Int-4 | 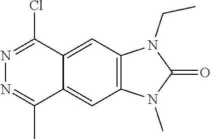 [SMILES: CCn1c(=O)n(C)c2cc3c(C)nnc(Cl)c3cc21] | UPLC-MS (ES⁺, Short acidic): 1.19 min, m/z 277.0 [M + H]⁺ (100%) |
| Int-5 | 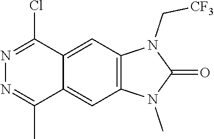 [SMILES: Cc1nnc(Cl)c2cc3c(cc12)n(C)c(=O)n3CC(F)(F)F] | UPLC-MS (ES⁺, Short acidic): 1.42 min, m/z 332.1 [M + H]⁺ (89%). |
| Int-6 | 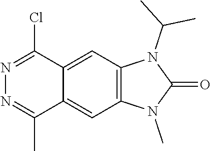 [SMILES: Cc1nnc(Cl)c2cc3c(cc12)n(C)c(=O)n3C(C)C] | UPLC-MS (ES⁺, Short acidic): 1.36 min, m/z 291.1 [M + H]⁺ (92%). |
| Int-7 | 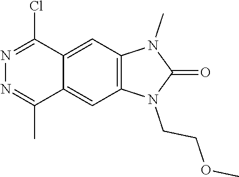 [SMILES: COCCn1c(=O)n(C)c2cc3c(Cl)nnc(C)c3cc21] | UPLC-MS (ES⁺, Short acidic): 1.22 min, m/z 307.1 [M + H]⁺ (88%) |
| Int-8 | 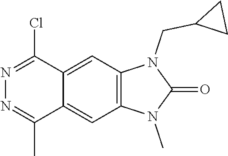 [SMILES: Cc1nnc(Cl)c2cc3c(cc12)n(C)c(=O)n3CC1CC1] | UPLC-MS (ES⁺, Short acidic): 1.40 min, m/z 303.1 [M + H]⁺ (93%) |
| Int-9 | 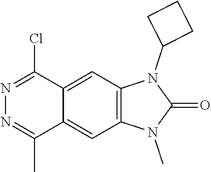 [SMILES: Cc1nnc(Cl)c2cc3c(cc12)n(C)c(=O)n3C1CCC1] | UPLC-MS (ES⁺, Short acidic): 1.48 min, m/z 303 [M + H]⁺ (82%). |
| Int-10 | 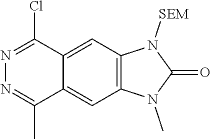 [SMILES: Cc1nnc(Cl)c2cc3c(cc12)n(C)c(=O)n3COCC[Si](C)(C)C] | UPLC-MS (ES⁺, Short acidic): 1.87 min, m/z 379.3 [M + H]⁺ (100%). |
| Int-11 | 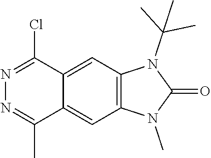 [SMILES: Cc1nnc(Cl)c2cc3c(cc12)n(C)c(=O)n3C(C)(C)C] | UPLC-MS (ES⁺, Short acidic): 1.49 min, m/z 306.2 [M + H]⁺ (98%) |
| Int-12 | 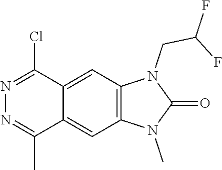 [SMILES: Cc1nnc(Cl)c2cc3c(cc12)n(C)c(=O)n3CC(F)F] | UPLC-MS (ES⁺, Short acidic): 1.20 min, m/z 313.0 [M + H]⁺ (94%) |

### Synthesis of 5-chloro-3-cyclopropyl-1,8-dimethyl-imidazo[4,5-g]phthalazin-2-one (Int-13)

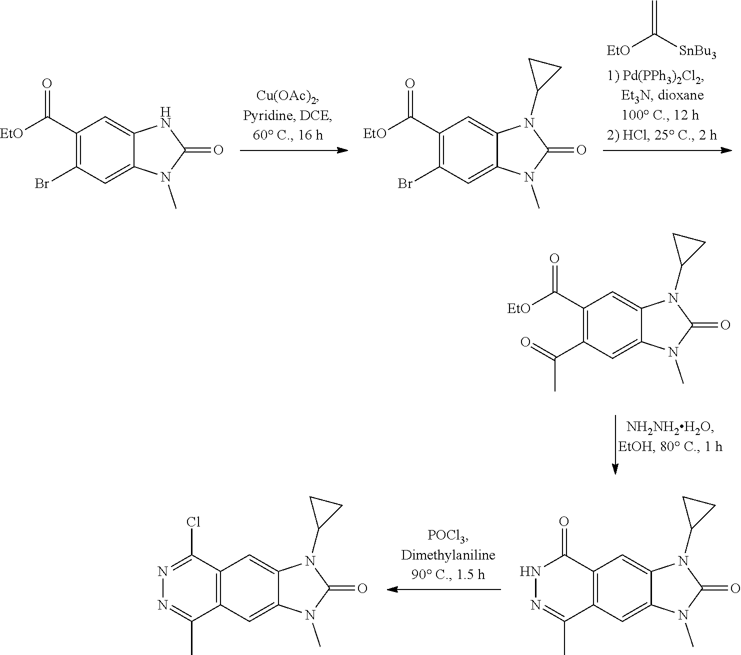

### Step 1

Copper(II) acetate (2.43 g, 13.4 mmol) was added to a stirring mixture of cyclopropylboronic acid (861.54 mg, 10.03 mmol), pyridine (1.62 mL, 20.1 mmol) and ethyl 6-bromo-1-methyl-2-oxo-3H-benzimidazole-5-carboxylate (2.00 g, 6.69 mmol) in DCE (30 mL). The reaction mixture was heated at 60° C. for 16 h. The RM was filtered through Celite washing with DCM. Water was added and the two phases were separated. The aqueous was re-extracted with DCM (2×). The combined organic extracts were passed through a phase separator and concentrated in vacuo. The crude material was purified by flash column chromatography (25 g, eluting in 0-100% EtOAc in pet ether) like fractions were pooled and concentrated in vacuo to afford ethyl 6-bromo-3-cyclopropyl-1-methyl-2-oxo-benzimidazole-5-carboxylate (1.77 g, 5.23 mmol, 78.2% yield) as a white solid.

UPLC-MS (ES⁺, Short acidic) 1.74 min, m/z 339.1 [M+H]⁺ (84%)

### Step 2

Made in the same way as 5-chloro-3-cyclopentyl-1,8-dimethyl-imidazo[4,5-g]phthalazin-2-one step 7. ethyl 6-acetyl-3-cyclopropyl-1-methyl-2-oxo-benzimidazole-5-carboxylate (1.28 g, 4.23 mmol, 80.9% yield) as a light yellow solid.

UPLC-MS (ES⁺, Short acidic): 1.50 min, m/z 303.1 [M+H]⁺ (100%)

¹H NMR (400 MHz, DMSO-d₆): δ 7.48 (s, 1H), 7.43 (s, 1H), 4.26 (q, J=7.1 Hz, 2H), 3.35 (s, 3H), 2.99-2.93 (m, 1H), 1.28 (t, J=7.1 Hz, 3H), 1.09-1.02 (m, 2H), 0.92-0.87 (m, 2H).

### Step 3

Made in the same way as 5-chloro-3-cyclopentyl-1,8-dimethyl-imidazo[4,5-g]phthalazin-2-one step 8. 1-cyclopropyl-3,5-dimethyl-7H-imidazo[4,5-g]phthalazine-2,8-dione (1.44 g, 5.31 mmol, 91.7% yield) as a white solid UPLC-MS (ES⁺, Short acidic): 1.16 min, m/z 271.0 [M+H]⁺ (100%) ¹H NMR (400 MHz, DMSO-d₆) δ 12.28 (s, 1H), 7.91 (s, 1H), 7.58 (s, 1H), 3.44 (s, 3H), 3.08-3.02 (m, 1H), 2.56 (s, 3H), 1.16-1.10 (m, 2H), 0.97-0.93 (m, 2H).

### Step 4

To 1-cyclopropyl-3,5-dimethyl-7H-imidazo[4,5-g]phthalazine-2,8-dione (500.mg, 1.85 mmol) was added phosphorus oxychloride (13.8 mL, 148 mmol) and dimethylaniline (0.47 mL, 3.70 mmol) and the reaction heated to 90° C. for 1.5 h. POCl₃ was removed under reduce pressure.

The crude was quenched with sat. aq. NaHCO₃ and the solid was filtrated and dried to afford 5-chloro-3-cyclopropyl-1,8-dimethyl-imidazo[4,5-g]phthalazin-2-one (520 mg, 1.51 mmol, 81.8% yield) as a grey/green solid.

UPLC-MS (ES⁺, short acidic): 1.23 min, m/z 289.0 [M+H]⁺ (100%).

¹H-NMR (400 MHz, DMSO-d₆): δ 7.90 (s, 1H), 7.77 (s, 1H), 3.49 (s, 3H), 3.13-3.07 (m, 1H), 2.92 (s, 3H), 1.19-1.13 (m, 2H), 1.02-0.96 (m, 2H)-contains 16% dimethylaniline

### Methyl 6-bromo-3-methyl-2-oxo-1H-benzimidazole-5-carboxylate (Int-14)

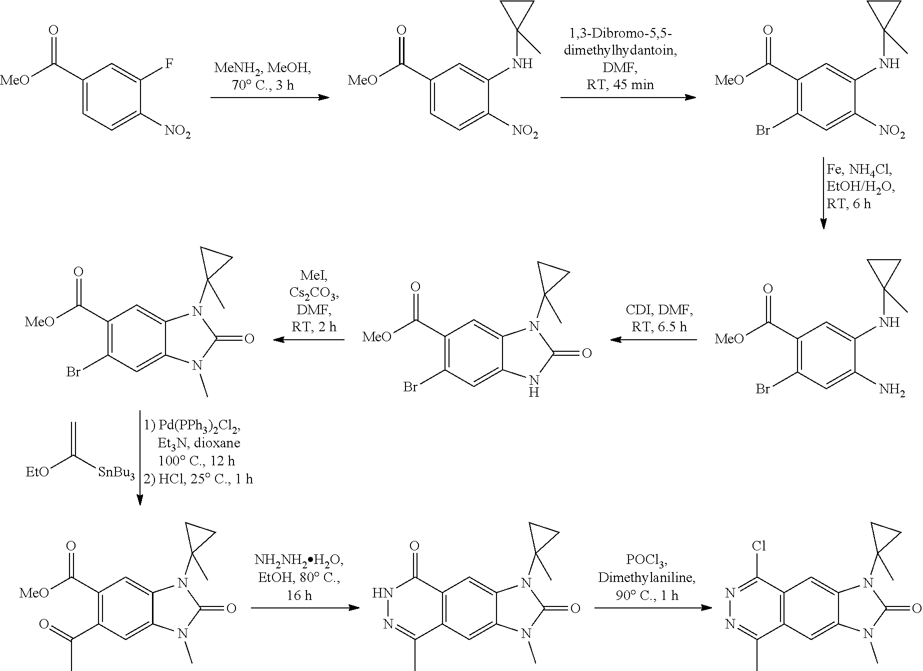

### Step 1

To a solution of 1-methylcyclopropanamine hydrochloride (1:1) (3.24 g, 30.1 mmol) in DMA (40 mL) at RT was added triethylamine (4.34 mL, 31.1 mmol) and stirred for 10 min. methyl 3-fluoro-4-nitrobenzoate (2.00 g, 10.0 mmol) was then added and the reaction mixture heated to 80° C. for 16 h.

The reaction mixture was cooled down to RT, diluted in EtOAc and washed with water and then brine (×3). The combined organic layers were passed through a hydrophobic filter and concentrated under reduced pressure. The crude product was purified by flash column chromatography (40 g, 0-100% EtOAc) affording methyl 3-[(1-methylcyclopropyl)amino]-4-nitro-benzoate (2.45 g, 9.78 mmol, 97.4% yield) as an orange solid.

UPLC-MS (ES⁺, Short acidic): 1.97 min, m/z 251.1 [M+H]⁺ (95%)

¹H NMR (400 MHz, DMSO-d₆) δ 8.23 (s, 1H), 8.17 (d, J=8.8 Hz, 1H), 7.91 (d, J=1.8 Hz, 1H), 7.21 (dd, J=1.8, 8.8 Hz, 1H), 3.91 (s, 3H), 1.41 (s, 3H), 0.90-0.82 (m, 4H).

### Step 2

To a solution of methyl 3-[(1-methylcyclopropyl)amino]-4-nitro-benzoate (1.20 g, 4.80 mmol) in DMF (32 mL) was added 1,3-Dibromo-5,5-dimethylhydantoin (1.17 g, 4.08 mmol) and the mixture was stirred at 25° C. for 45 min. The reaction mixture was partitioned between EtOAc and water. The organic layer was separated. The aqueous layer was extracted with EtOAc (×3). The organic layers were combined, washed with brine (×3), passed through a hydrophobic paper and concentrated under reduced pressure. The crude product was purified by normal phase chromatography (25 g, 0-25% EtOAc in pet ether) to give methyl 2-bromo-5-[(1-methylcyclopropyl)amino]-4-nitro-benzoate (781 mg, 2.37 mmol, 49.5% yield) as an orange solid.

UPLC-MS (ES⁺, Short acidic): 2.05 min, m/z 330.9 [M+H]⁺ (100%)

¹H NMR (400 MHz, DMSO-d₆) δ 8.29-8.27 (m, 2H), 7.61 (s, 1H), 3.92 (s, 3H), 1.38 (s, 3H), 0.86-0.83 (m, 4H)

### Step 3

A mixture of methyl 2-bromo-5-[(1-methylcyclopropyl)amino]-4-nitro-benzoate (940 mg, 2.86 mmol), iron powder (797 mg, 14.3 mmol) and ammonium chloride (1.37 g, 25.7 mmol) in Ethanol (9.5 mL)/Water (2.4 mL) was stirred at RT for 6.5 h.

The mixture was cooled to room temperature and passed through a pad of celite, washing with MeOH. Filtrated then concentrated in vacuo. EtOAc and water was added to the aqueous mixture and the layers separated. The aqueous was extracted with EtOAc (3×). The combined organics were washed with brine, passed through a hydrophobic filter and concentrated in vacuo to afford methyl 4-amino-2-bromo-5-[(1-methylcyclopropyl)amino]benzoate (788 mg, 2.63 mmol, 92.2% yield) as a brown oil.

UPLC-MS (ES⁺, Short acidic): 1.70 min, m/z 299.0 [M+H]⁺ (79%) -

¹H NMR (400 MHz, DMSO-d₆) δ 7.27 (s, 1H), 6.76 (s, 1H), 5.48 (s, 2H), 5.31 (s, 1H), 3.75 (s, 3H), 1.29 (s, 3H), 0.65 (s, 4H)

### Step 4

To a vial methyl 4-amino-2-bromo-5-[(1-methylcyclopropyl)amino]benzoate (788 mg, 2.63 mmol) in DMF (16.5 mL) and 1,1′-Carbonyldiimidazole (1.28 g, 7.90 mmol) was added. The vial was sealed and stirred at RT for 6 h 30 min. The reaction mixture was cooled to 0° C. and Ice-water was added very slowly. The resulting solid was filtered and washed with water to give methyl 6-bromo-3-(1-methylcyclopropyl)-2-oxo-1H-benzimidazole-5-carboxylate (448 mg, 1.38 mmol, 52.3% yield) as a light orange/brown solid.

UPLC-MS (ES⁺, Short acidic): 1.51 min, m/z 327.0 [M+H]⁺ (67%)

¹H NMR (400 MHz, DMSO-d₆) δ 11.22 (s, 1H), 7.54 (s, 1H), 7.24 (s, 1H), 3.86 (s, 3H), 1.38 (s, 3H), 1.04-0.94 (m, 4H)

### Step 5

To a vigorously stirring suspension of methyl 6-bromo-3-(1-methylcyclopropyl)-2-oxo-1H-benzimidazole-5-carboxylate (535.mg, 1.65 mmol), Cesium Carbonate (0.8 g, 2.47 mmol) in DMF (8.2 mL) was added Iodomethane (154 μL, 2.47 mmol). The reaction mixture stirred at RT for 2 hr.

Water and DCM were slowly added. The two phases were separated, the aqueous phase extracted with DCM (×3). The combined organic layers were washed with water, brine, passed through a hydrophobic filter and concentrated under reduced pressure to afford methyl 6-bromo-1-methyl-3-(1-methylcyclopropyl)-2-oxo-benzimidazole-5-carboxylate (540 mg, 1.59 mmol, 96.7% yield) as a light brown solid.

UPLC-MS (ES⁺, Short acidic): 1.67 min, m/z 340.9 [M+H]⁺ (62%)

¹H NMR (400 MHz, DMSO-d₆): 7.57-7.56 (m, 2H), 3.86 (s, 3H), 3.31 (s, 3H), 1.38 (s, 3H), 1.05-0.95 (m, 4H)

### Step 6

To a degassed solution of methyl 6-bromo-1-methyl-3-(1-methylcyclopropyl)-2-oxo-benzimidazole-5-carboxylate (539 mg, 1.59 mmol), Tributyl(1-ethoxyvinyl)tin (0.64 mL, 1.91 mmol), Triethylamine (0.55 mL, 3.97 mmol) in 1,4-Dioxane (10.6 mL) was added Bis(triphenylphosphine)palladium(II) dichloride (112 mg, 0.16 mmol). The reaction mixture was heated to 100° C. for 16 h. The reaction mixture was cooled to rt and hydrogen chloride (2.9 mL, 5.84 mmol) was added. The reaction mixture was stirred for 1 h. Water and EtOAc were added, and the two phases were separated. The aqueous was extracted with EtOAc (3×). The combined organic extracts were washed with brine, passed through hydrophobic filter paper and concentrated in vacuo. The crude residue was purified by flash chromatography (12 g, 0-100% EtOAc in petroleum ether) to afford methyl 6-acetyl-1-methyl-3-(1-methylcyclopropyl)-2-oxo-benzimidazole-5-carboxylate (341 mg, 1.13 mmol, 71.1% yield) as light brown solid.

UPLC-MS (ES⁺, Short acidic): 1.45 min, m/z 303.1 [M+H]⁺ (97%)

¹H NMR (400 MHz, DMSO-d₆) δ 7.50 (s, 1H), 7.45 (s, 1H), 3.80 (s, 3H), 3.35 (s, 3H), 1.39 (s, 3H), 1.04-0.99 (m, 4H) one CH₃ under the DMSO signal

### Step 7

To a stirring solution of methyl 6-acetyl-1-methyl-3-(1-methylcyclopropyl)-2-oxo-benzimidazole-5-carboxylate (341 mg, 1.13 mmol) in Ethanol (8.4 mL) was added Hydrazine Hydrate (82 μL, 1.69 mmol). The reaction mixture was heated to 80° C. for 16 h. The reaction mixture was concentrated under reduced pressure, the solid was washed with MTBE, dried in vacuo to afford 3,5-dimethyl-1-(1-methylcyclopropyl)-7H-imidazo[4,5-g]phthalazine-2,8-dione (251 mg, 0.88 mmol, 78.3% yield) as a grey solid.

UPLC-MS (ES⁺, Short acidic): 1.21 min, m/z 285.1 [M+H]⁺ (100%)

¹H NMR (400 MHz, DMSO-d₆) δ 12.29 (s, 1H), 7.90 (s, 1H), 7.59 (s, 1H), 3.44 (s, 3H), 2.55 (s, 3H), 1.44 (s, 3H), 1.12-1.04 (m, 4H)

### Step 8

To 3,5-dimethyl-1-(1-methylcyclopropyl)-7H-imidazo[4,5-g]phthalazine-2,8-dione (61 mg, 0.21 mmol) was added phosphorus oxychloride (1.6 mL, 17.2 mmol) and dimethylaniline (54 μL, 0.43 mmol) and the reaction heated to 90° C. for 1 h. POCl₃ was removed under reduce pressure.

The crude was quenched with sat. aq. NaHCO₃ and the solid collected by vacuum filtration and dried to afford 5-chloro-1,8-dimethyl-3-(1-methylcyclopropyl)imidazo[4,5-g]phthalazin-2-one (quantitative yield) as an grey/green solid.

UPLC-MS (ES⁺, short acidic): 1.33 min, m/z 303.1 [M+H]⁺ (100%).

¹H-NMR (400 MHz, DMSO-d₆) δ 7.91 (s, 1H), 7.76 (s, 1H), 3.49 (s, 3H), 2.92 (s, 3H), 1.48 (s, 3H), 1.16-1.07 (m, 4H)

### 5-chloro-1-cyclopropyl-3,8-dimethyl-imidazo[4,5-g]phthalazin-2-one (Int-15)

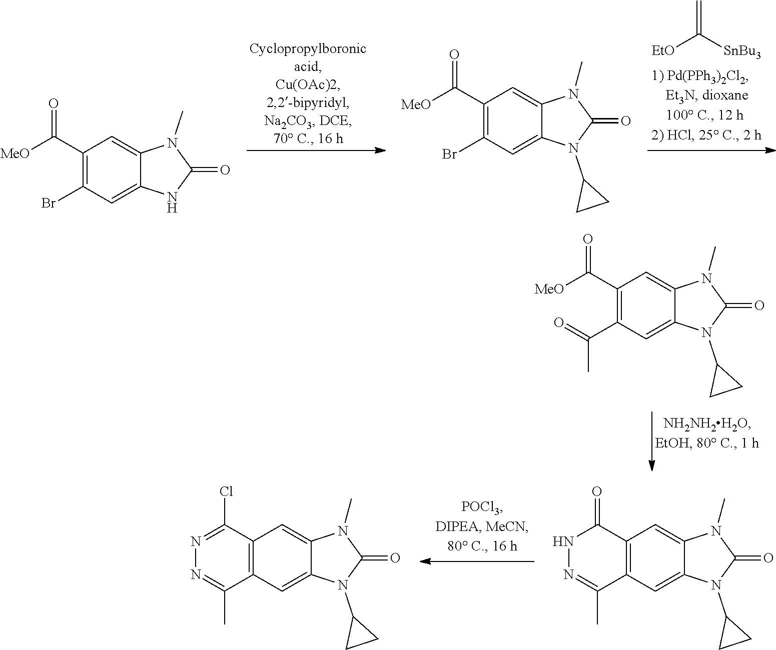

### Step 1

A mixture of methyl 6-bromo-3-methyl-2-oxo-1H-benzimidazole-5-carboxylate (200 mg, 0.60 mmol), Copper(II) acetate (114 mg, 0.63 mmol), cyclopropylboronic acid (102 mg, 1.19 mmol), 2,2′-bipyridyl (98 mg, 0.63 mmol), sodium carbonate (139 mg, 1.31 mmol) in DCE (3 mL) was stirred for 16 h at 70° C.

The reaction was quenched with sat. aq. NH₄Cl, diluted with DCM, filtered. Filtrate extracted with DCM (×3). Organic layers were combined, washed with brine, passed through a phase separator and concentrated in vacuo. The crude product was purified by flash column chromatography (12 g, 0-100% EtOAc in pet ether), to afford methyl 6-bromo-1-cyclopropyl-3-methyl-2-oxo-benzimidazole-5-carboxylate (138 mg, 0.42 mmol, 71.2% yield) as a cream solid.

UPLC-MS (ES⁺, Short acidic): 1.58 min, m/z 327.0[M+H]⁺ (81%)

1H NMR (400 MHz, CDCl₃): 7.47 (s, 1H), 7.45 (s, 1H), 3.94 (s, 3H), 3.39 (s, 3H), 2.90-2.83 (m, 1H), 1.17-1.10 (m, 2H), 1.04-0.97 (m, 2H)

### Step 2

Made in the same way as methyl 6-bromo-3-methyl-2-oxo-1H-benzimidazole-5-carboxylate Step 6. Methyl 6-acetyl-1-cyclopropyl-3-methyl-2-oxo-benzimidazole-5-carboxylate (264 mg, 0.92 mmol, 79.2% yield) as a white solid.

UPLC-MS (ES⁺, Short acidic): 1.43 min, m/z 289.0 [M+H]⁺ (98%)

1H NMR (400 MHz, CDCl₃) 7.45 (s, 1H), 7.18 (s, 1H), 3.91 (s, 3H), 3.43 (s, 3H), 2.93-2.87 (m, 1H), 2.55 (s, 3H), 1.18-1.12 (m, 2H), 1.05-0.99 (m, 2H)

### Step 3

Made in the same way as methyl 6-bromo-3-methyl-2-oxo-1H-benzimidazole-5-carboxylate Step 7. 3-cyclopropyl-1,5-dimethyl-7H-imidazo[4,5-g]phthalazine-2,8-dione (150 mg, 0.56 mmol, 60.1% yield) as a grey solid.

UPLC-MS (ES⁺, Short acidic): 1.25 min, m/z 271.0 [M+H]⁺ (100%)

1H NMR (400 MHz, DMSO-d₆): 12.26 (s, 1H), 7.87 (s, 1H), 7.49 (s, 1H), 3.42 (s, 3H), 3.06-2.99 (m, 1H), 2.56 (s, 3H), 1.16-1.09 (m, 2H), 1.00-0.95 (m, 2H)

### Step 4

3-cyclopropyl-1,5-dimethyl-7H-imidazo[4,5-g]phthalazine-2,8-dione (152 mg, 0.56 mmol) is suspended in MeCN (2 mL). Phosphorus oxychloride (0.52 mL, 5.62 mmol) N,N-Diisopropylethylamine (0.49 mL, 2.81 mmol) was added and the reaction was stirred at 80° C. for 16 h. RM cooled to rt and more phosphorus oxychloride (0.26 mL, 2.81 mmol) added. After 2 hr, cooled to rt. All volatiles removed under reduced pressure. Reside dissolved in DCM, and added to ice-cold NaHCO₃ until pH 8. Layers separated. Aq. extracted ×3 with DCM. Combined organics washed with brine, passed through hydrophobic frit and concentrated under reduced pressure to give 5-chloro-1-cyclopropyl-3,8-dimethyl-imidazo[4,5-g]phthalazin-2-one (141 mg, 0.49 mmol, 86.8% yield) as a brown solid

UPLC-MS (ES⁺, Short acidic): 1.28 min, m/z 289.0 [M+H]⁺ (97%)

1H NMR (400 MHz, DMSO-d₆)-7.80 (s, 1H), 7.76 (s, 1H), 3.48 (s, 3H), 3.10-3.04 (m, 1H), 2.94 (s, 3H), 1.20-1.13 (m, 2H), 1.05-0.99 (m, 2H)

### 6-chloro-4,9-dimethyl-pyridazino[4,5-g][1,4]benzoxazin-3-one (Int-16)

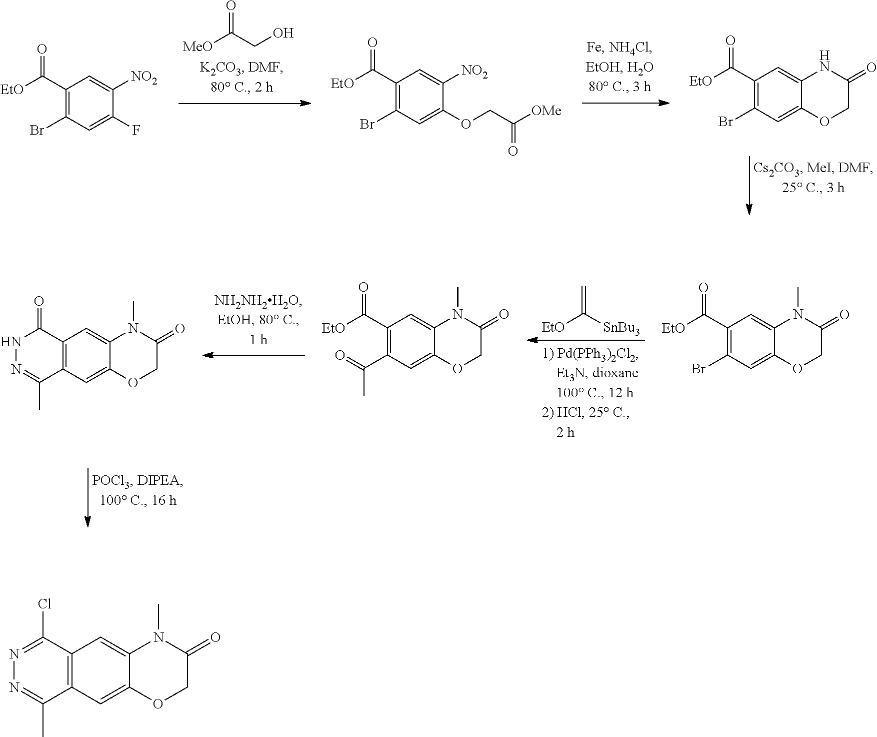

### Step 1

To a solution of ethyl 2-bromo-4-fluoro-5-nitro-benzoate (1.06 g, 3.62 mmol) in DMF (10 mL) was added Potassium carbonate (1.50 g, 10.9 mmol) and Methyl glycolate (0.31 mL, 3.98 mmol). The reaction was heated to 80° C. for 2 h. The reaction mixture was concentrated in vacuo.

The residue was taken up in EtOAc and Water. The two phases were separated and the aqueous was extracted with EtOAc (2×). The combined organic layers were dried over Na₂SO₄ and concentrated. The crude material was purified by column chromatography (12 g, 0-50% EtOAc in petroleum ether) to yield ethyl 2-bromo-4-(2-methoxy-2-oxo-ethoxy)-5-nitro-benzoate (316 mg, 0.87 mmol, 24% yield) as a yellow solid.

UPLC-MS (ES⁺, Short acidic): 1.78 min, m/z 361.9/363.9 [M+H]⁺ (89%)

¹H NMR (400 MHz, CDCl₃) 8.47 (s, 1H), 7.24 (s, 1H), 4.84 (s, 2H), 4.41 (q, J=7.2 Hz, 2H), 3.84 (s, 3H), 1.41 (t, J=7.2 Hz, 3H).

### Step 2

Ethyl 2-bromo-4-(2-methoxy-2-oxo-ethoxy)-5-nitro-benzoate (256 mg, 0.71 mmol) was suspended in Ethanol (4 mL) and water (1 mL) followed by the addition of iron (237 mg, 4.24 mmol) and ammonium chloride (303 mg, 5.66 mmol). The reaction mixture was heated at 80° C. for 3 h. Reaction was cooled to rt, filtered over celite and concentrated. The crude was partitioned between water and EtOAc. The two phases were separated. The aqueous phase was extracted with EtOAc (×3). The organic phases were combined, washed with brine, dried over Na₂SO₄ and concentrates to afford ethyl 7-bromo-3-oxo-4H-1,4-benzoxazine-6-carboxylate (187 mg, 0.62 mmol, 88.1% yield) as a pale yellow solid.

UPLC-MS (ES⁺, Short acidic): 1.59 min, m/z 299.9/301.9 [M+H]⁺.

1H NMR (400 MHz, CDCl₃): δ 7.60 (br s, 1H), 7.35 (s, 1H), 7.29 (s, 1H), 4.68 (s, 2H), 4.38 (q, J=7.2 Hz, 2H), 1.40 (t, J=7.2 Hz, 3H)

### Step 3

To a stirring suspension of ethyl 7-bromo-3-oxo-4H-1,4-benzoxazine-6-carboxylate (187.mg, 0.62 mmol) and cesium carbonate (510 mg, 1.56 mmol) in DMF (5 mL) was added Iodomethane (0.06 mL, 0.93 mmol) and the reaction was stirred at RT for 3 hrs. The reaction was concentrated. Water and EtOAc were added. The two phases were separated. The aqueous was extracted with EtOAc (×3). Combined organics were washed with brine, dried over Na₂SO₄, filtered and concentrated. The crude material was purified by flash column chromatography (0-50% EtOAc in petroleum ether) to yield ethyl 7-bromo-4-methyl-3-oxo-1,4-benzoxazine-6-carboxylate (152 mg, 0.48 mmol, 77.7% yield) as a pale yellow solid.

UPLC-MS (ES⁺, Short acidic): 1.72 min, m/z 313.9/315.9 [M+H]⁺ (97%)

¹H NMR (400 MHz, CDCl₃): δ 7.48 (s, 1H), 7.27 (s, 1H), 4.68 (s, 2H), 4.40 (q, J=7.2 Hz, 2H), 3.39 (s, 3H), 1.42 (t, J=7.2 Hz, 3H).

### Step 4

To a stirring, thoroughly degassed solution of, ethyl 7-bromo-4-methyl-3-oxo-1,4-benzoxazine-6-carboxylate (152 mg, 0.48 mmol), tributyl(1-ethoxyvinyl)tin (0.20 mL, 0.58 mmol) and triethylamine (0.17 mL, 1.21 mmol) in 1,4-Dioxane (6.2 mL) was added bis(triphenylphosphine)palladium(II) dichloride (34 mg, 0.05 mmol). The reaction mixture was heated at 100° C. for 2 h. The reaction was cooled to rt. 2M hydrogen chloride aq. solution (1.21 mL, 2.42 mmol) was added and the reaction mixture was stirred for 1 h at rt. Water and EtOAc were added, and the layers separated. The aqueous was extracted with EtOAc (×3). The combined organics were washed with brine, dried over Na₂SO₄, filtered and concentrated. The crude material was purified by column chromatography (0-100% EtOAc in petroleum ether) to yield ethyl 7-acetyl-4-methyl-3-oxo-1,4-benzoxazine-6-carboxylate (100 mg, 0.36 mmol, 74.5% yield) as an off-white solid.

UPLC-MS (ES⁺, Short acidic): 1.47 min, m/z 278.1 [M+H]⁺ (96%).

1H-NMR (400 MHz, CDCl₃): 7.47 (s, 1H), 6.99 (s, 1H), 4.70 (s, 2H), 4.36 (q, J=7.1 Hz, 2H), 3.42 (s, 3H), 2.51 (s, 3H), 1.37 (t, J=7.1 Hz, 3H).

### Step 5

To a stirring suspension of ethyl 7-acetyl-4-methyl-3-oxo-1,4-benzoxazine-6-carboxylate (100 mg, 0.36 mmol) in Ethanol (2.3 mL) was added hydrazine hydrate (26 μL, 0.54 mmol). The reaction mixture was heated at 80° C. After 1 hour, all volatiles were removed. The residue was washed with MTBE, filtered, and dried under vacuum to yield 4,9-dimethyl-7H-pyridazino[4,5-g][1,4]benzoxazine-3,6-dione (87.2 mg, 0.36 mmol, 98.6% yield) as a grey solid.

UPLC-MS (ES⁺, Short acidic): 1.16 min, m/z 246.1 [M+H]⁺ (100%).

1H-NMR (400 MHz, DMSO-d₆): δ 12.35 (s, 1H), 7.81 (s, 1H), 7.46 (s, 1H), 4.88 (s, 2H), 3.41 (s, 3H), 2.46 (s, 3H).

### Step 6

To a solution of 4,9-dimethyl-7H-pyridazino[4,5-g][1,4]benzoxazine-3,6-dione (87 mg, 0.36 mmol) in MeCN (2.5 mL) was added N,N-Diisopropylethylamine (0.31 mL, 1.78 mmol) and phosphorus oxychloride (0.33 mL, 3.56 mmol). The reaction mixture was heated at 80° C. overnight. The reaction was cooled to RT. Sat. aq. NaHCO₃ was added till neutral pH followed by extraction with EtOAc (×3) and DCM/MeOH (×3). The combined organics were washed with brine, dried over Na₂SO₄, filtered and concentrated to yield 6-chloro-4,9-dimethyl-pyridazino[4,5-g][1,4]benzoxazin-3-one (93.8 mg, 0.36 mmol, 100% yield) as a brown solid.

UPLC-MS (ES⁺, Short acidic): 1.24 min, m/z 264.0/265.9 [M+H]⁺ (60%)

The following chlorophthalazines were prepared in a similar manner.

TABLE 3B
Intermediates Prepared According to the Disclosed Methods

| Example | Structure | Analytical data |
|---|---|---|
| Int-17 | 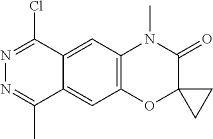 [SMILES: Cc1nnc(Cl)c2cc3c(cc12)OC1(CC1)C(=O)N3C] | UPLC-MS (ES⁺, Short acidic): 1.48 min, m/z 290.1 [M + H]⁺ (93%) |
| Int-18 | 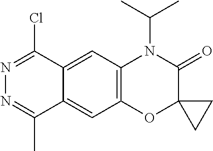 [SMILES: Cc1nnc(Cl)c2cc3c(cc12)OC1(CC1)C(=O)N3C(C)C] | UPLC-MS (ES⁺, Short acidic): 1.72 min, m/z 318.2 [M + H]⁺ (100%) |
| Int-19 | 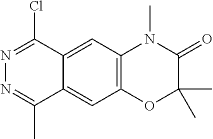 [SMILES: Cc1nnc(Cl)c2cc3c(cc12)OC(C)(C)C(=O)N3C] | UPLC-MS (ES⁺, Short acidic): 1.50 min, m/z 292.1 [M + H]⁺ (100%) |

### 7-chloro-5,10-dimethyl-2,3-dihydropyridazino[4,5-h][1,5]benzoxazepin-4-one (Int-20)

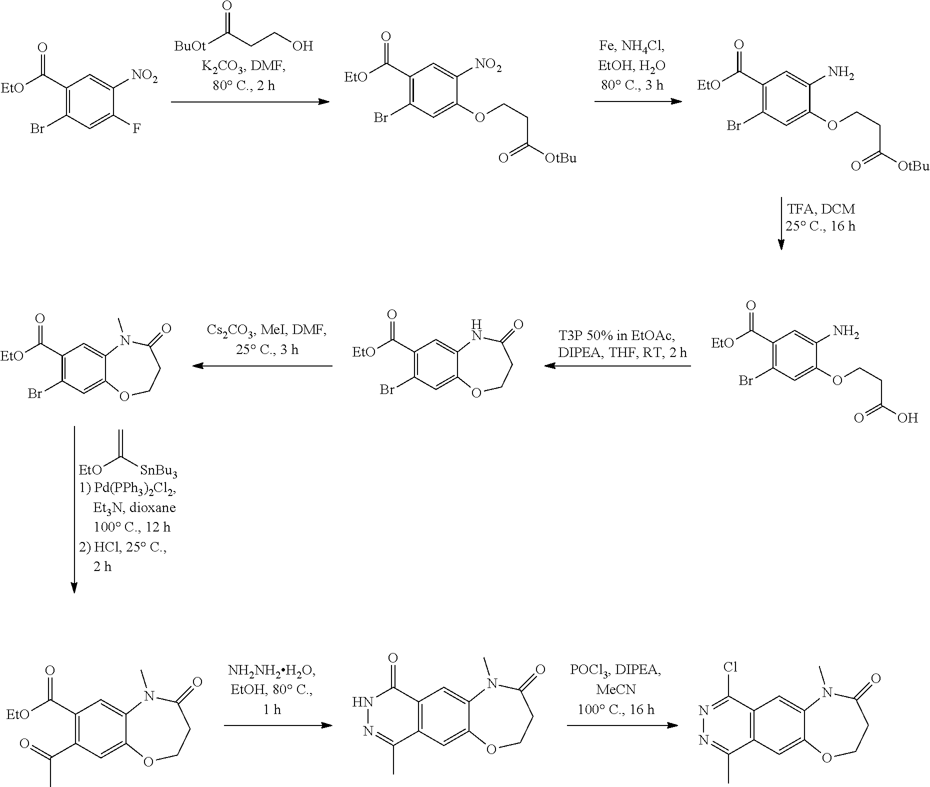

### Step 1

To a vial containing ethyl 2-bromo-4-fluoro-5-nitro-benzoate (3 g, 10.3 mmol) in DMF (10 mL) was added potassium carbonate (4.3 g, 30.8 mmol), tert-butyl 3-hydroxypropionate (1.67 mL, 11.3 mmol) and molecular sieves. The vial was sealed and heated at 80° C. overnight. The reaction mixture was filtered and concentrated in vacuo. The crude material was purified by flash column chromatography (40 g, eluting in 0-60% EtOAc in pet ether) like fractions were pooled and concentrated in vacuo to afford ethyl 2-bromo-4-(3-tert-butoxy-3-oxo-propoxy)-5-nitro-benzoate (1.47 g, 3.50 mmol, 34.1% yield) as a yellow solid.

UPLC-MS (ES−, Short acidic): 2.06 min, m/z 288.0/290.1 [M−H]⁻ (77%)

1H NMR (400 MHz, CDCl3): δ 8.44 (s, 1H), 7.41 (s, 1H), 4.42-4.36 (m, 2H), 2.79 (t, J=6.2 Hz, 2H), 1.47 (s, 9H), 1.44-1.38 (m, 5H)

### Step 2

A mixture of ethyl 2-bromo-4-(3-tert-butoxy-3-oxo-propoxy)-5-nitro-benzoate (1.47 g, 3.5 mmol), iron powder (978 mg, 17.5 mmol) and ammonium chloride (1.50 g, 28.1 mmol) in ethanol (13.0 mL)/water (3.00 mL) was heated to 80° C. for 2 hrs. Reaction was cooled to RT, passed through a syringe filter (flushing with MeOH) and concentrated. Crude residue partitioned between water and EtOAc. Two phases were separated and the aqueous was re-extracted with EtOAc. Combined organics were washed with brine, passed through phase separating filter paper and concentrated in vacuo to afford ethyl 5-amino-2-bromo-4-(3-tert-butoxy-3-oxo-propoxy)benzoate (964 mg, 2.48 mmol, 70.9% yield) as an orange oil.

UPLC-MS (ES⁺, Short acidic): 2.04 min, m/z 388.1, 390.1 [M+H]⁺ (94%)

1H NMR (400 MHz, CDCl₃) 7.24 (s, 1H), 7.02 (s, 1H), 4.34 (q, J=7.1 Hz, 2H), 4.26 (t, J=6.2 Hz, 2H), 2.74 (t, J=6.2 Hz, 2H), 1.46 (s, 9H), 1.38 (t, J=7.1 Hz, 3H)

### Step 3

To a solution of ethyl 5-amino-2-bromo-4-(3-tert-butoxy-3-oxo-propoxy)benzoate (266 mg, 0.69 mmol) in DCM (3 mL) was added trifluoroacetic acid (0.16 mL, 2.06 mmol) and the reaction mixture was stirred at RT overnight. The reaction mixture was concentrated in vacuo to afford 3-(2-amino-5-bromo-4-ethoxycarbonyl-phenoxy)propanoic acid; 2,2,2-trifluoroacetic acid (255 mg, 0.57 mmol, 83.4% yield) as a brown waxy solid. This material was telescoped through to the next step without further purification.

UPLC-MS (ES⁺, Short acidic): 1.56 min, m/z 333.9 [M+H]⁺ (69%)

### Step 4

Propylphosphonic anhydride 50% solution in EtOAc (5.91 mL, 9.92 mmol) was added dropwise to a stirred solution of N,N-diisopropylethylamine (4.32 mL, 24.8 mmol) and 3-(2-amino-5-bromo-4-ethoxycarbonyl-phenoxy)propanoic acid (824.mg, 2.48 mmol) in THF (10 mL) the reaction was stirred for 2 hours. The reaction mixture was quenched with NaHCO₃ sat solution and EtOAc was added. The two phases were separated and the aqueous was re-extracted with EtOAc (2×). The combined organic extracts were passed through phase separating filter paper and concentrated in vacuo. The crude material was purified by flash column chromatography (12 g, eluting in 0-100% EtOAc in pet ether) like fractions were pooled and concentrated in vacuo to afford ethyl 8-bromo-4-oxo-3,5-dihydro-2H-1,5-benzoxazepine-7-carboxylate (358 mg, 1.14 mmol, 45.9% yield) as a pale yellow solid.

UPLC-MS (ES⁺, Short acidic): 1.55 min, m/z 314.0, 316.0 [M+H]⁺ (97%)

1H NMR (400 MHz, DMSO) 9.97 (s, 1H), 7.64 (s, 1H), 7.31 (s, 1H), 4.44-4.40 (m, 2H), 4.29 (q, J=7.1 Hz, 2H), 2.83-2.79 (m, 2H), 1.31 (t, J=7.1 Hz, 3H)

### Step 5

To a vigorously stirring suspension of ethyl 8-bromo-4-oxo-3,5-dihydro-2H-1,5-benzoxazepine-7-carboxylate (358.mg, 1.14 mmol) Cesium Carbonate (557 mg, 1.71 mmol) in DMF (4 mL) was added Iodomethane (106.uL, 1.71 mmol). The reaction mixture was stirred at RT for 1 hr. The reaction mixture was partitioned between water and EtOAc. The two phases were separated and the aqueous was re-extracted with EtOAc (2×). Combined organic extracts were washed with brine, passed through phase separating filter paper and concentrated in vacuo to afford ethyl 8-bromo-5-methyl-4-oxo-2,3-dihydro-1,5-benzoxazepine-7-carboxylate (308 mg, 0.94 mmol, 82.4% yield) as a brown solid.

UPLC-MS (ES⁺, Short acidic): 1.79 min, m/z 328.0, 329.0 [M+H]⁺ (97%)

1H NMR (400 MHz, CDCl₃) 7.67 (s, 1H), 7.41 (s, 1H), 4.63 (t, J=6.5 Hz, 2H), 4.41 (q, J=7.1 Hz, 2H), 3.35 (s, 3H), 2.68 (t, J=6.5 Hz, 2H), 1.42 (t, J=7.1 Hz, 3H)

### Step 6

A solution of Tributyl(1-ethoxyvinyl)tin (0.38 mL, 1.13 mmol), ethyl 8-bromo-5-methyl-4-oxo-2,3-dihydro-1,5-benzoxazepine-7-carboxylate (308.mg, 0.94 mmol), triethylamine (0.33 mL, 2.35 mmol) in 1,4-Dioxane (5 mL) was degassed with nitrogen for 5 mins.

Bis(triphenylphosphine)palladium(II) dichloride (66 mg, 0.09 mmol) was added and the mixture was degassed for a further 5 mins. The reaction was heated at 100° C. overnight. The reaction was cooled down to RT and hydrogen chloride (2M aqueous solution) (2.35 mL, 4.69 mmol) was added. After 1 h, reaction was complete. The solution was then partitioned between water and EtOAc. Aq. layer extracted three times with EtOAc. Combined organics passed through phase-separating filter paper, and concentrated in vacuo. The crude material was purified by flash column chromatography (4 g, eluting in 0-100% EtOAc in pet ether) like fractions were pooled and concentrated in vacuo to afford ethyl 8-acetyl-5-methyl-4-oxo-2,3-dihydro-1,5-benzoxazepine-7-carboxylate (158 mg, 0.54 mmol, 57.8% yield) as a yellow oil.

UPLC-MS (ES⁺, Short acidic): 1.54 min, m/z 292.0 [M+H]⁺ (90%)

1H NMR (400 MHz, CDCl₃) δ 7.69 (s, 1H), 7.13 (s, 1H), 4.65 (t, J=6.5 Hz, 2H), 4.38 (q, J=7.1 Hz, 2H), 3.38 (s, 3H), 2.69 (t, J=6.5 Hz, 2H), 2.53 (s, 3H), 1.38 (t, J=7.2 Hz, 3H)

### Step 7

Ethyl 8-acetyl-5-methyl-4-oxo-2,3-dihydro-1,5-benzoxazepine-7-carboxylate (158.mg, 0.54 mmol) and Hydrazine Hydrate (40 uL, 0.81 mmol) were mixed in ethanol (3 mL). The reaction mixture was heated to 80° C. overnight. The reaction mixture was concentrated in vacuo. The solid was washed with MTBE and dried to afford 5,10-dimethyl-3,8-dihydro-2H-pyridazino[4,5-h][1,5]benzoxazepine-4,7-dione (122 mg, 0.47 mmol, 86.8% yield) as an orange solid.

UPLC-MS (ES⁺, Short acidic): 1.25 min, m/z 260.0 [M+H]⁺ (100%) 1H NMR (400 MHz, DMSO) 12.43 (s, 1H), 8.11 (s, 1H), 7.65 (s, 1H), 4.62 (t, J=6.6 Hz, 2H), 3.35 (s, 3H), 2.66 (t, J=6.6 Hz, 2H), 2.49 (s, 3H)

### Step 8

To a solution of 5,10-dimethyl-3,8-dihydro-2H-pyridazino[4,5-h][1,5]benzoxazepine-4,7-dione (122.mg, 0.47 mmol) in MeCN (3 mL) was added phosphorus oxychloride (439 uL, 4.71 mmol) and N,N-Diisopropylethylamine (410 uL, 2.35 mmol). The reaction mixture was heated at 80° C. for 19 hours. The reaction mixture was concentrated in vacuo. The residue was then taken up in DCM and NaHCO₃ sat solution. The two phases were separated and the aqueous layer was re-extracted with DCM. The combined organic extracts were filtered through phase separating filter paper and concentrated in vacuo to afford 7-chloro-5,10-dimethyl-2,3-dihydropyridazino[4,5-h][1,5]benzoxazepin-4-one (Quantitative yield) as a black solid. The material was used in the next step without further purification.

UPLC-MS (ES⁺, Short acidic): 1.34 min, m/z 278.0/280 [M+H]⁺ (97%)

### 8-chloro-1,3,3,5-tetramethyl-pyrrolo[3,2-g]phthalazin-2-one (Int-21)

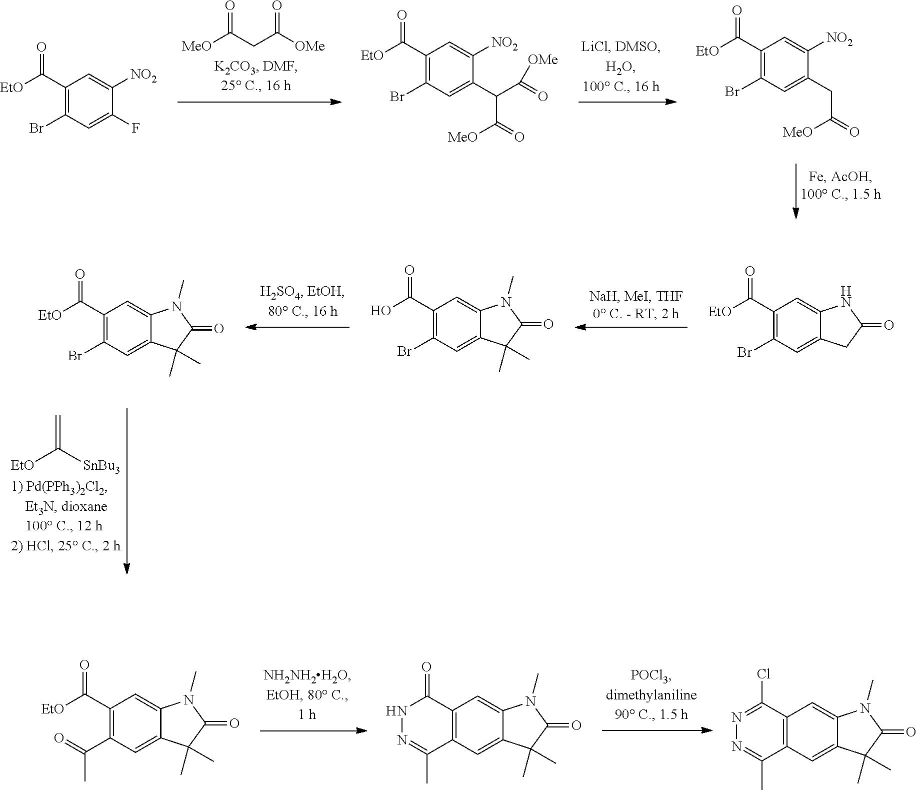

### Step 1

To a cooled solution at 0° C. of Dimethylmalonate (2.36 mL, 20.5 mmol) in DMF (33 mL) was added ethyl 2-bromo-4-fluoro-5-nitro-benzoate (4.g, 13.7 mmol) and Potassium carbonate (5.68 g, 41.1 mmol). The reaction was stirred at rt overnight. Water was added and the reaction mixture was extracted with EtOAc (3×). The combined organic extracts were washed with Brine and concentrated in vacuo. The resulting solid was washed with minimal methanol and dried by vacuum filtration to afford dimethyl 2-(5-bromo-4-ethoxycarbonyl-2-nitro-phenyl)propanedioate (36.8 g, 91.2 mmol, 76.1% yield) as a pale yellow solid.

UPLC-MS (ES⁺, Short acidic): 1.85 min, m/z 404.0/405.9 [M+H]⁺ (97%).

1H-NMR (400 MHz, CDCl₃): δ 8.51 (s, 1H), 7.84 (s, 1H), 5.21 (s, 1H), 4.45 (q, J=7.1 Hz, 2H), 3.82 (s, 6H), 1.43 (t, J=7.2 Hz, 3H)

### Step 2

A solution of dimethyl 2-(5-bromo-4-ethoxycarbonyl-2-nitro-phenyl)propanedioate (4.73 g, 11.7 mmol), lithium chloride (993 mg, 23.4 mmol) in DMSO (47 mL) and water (27 mL) was heated to 100° C. for 16H. The reaction was cooled to rt and poured into ice-water. The resulting precipitate was collected and dried by vacuum filtration to afford ethyl 2-bromo-4-(2-methoxy-2-oxo-ethyl)-5-nitro-benzoate (23.4 g, 67.5 mmol, 93% yield) as a pale yellow solid.

UPLC-MS (ES−, short acidic): 1.86 min, m/z 344.2, 346.1 [M−H]⁻ (86%),

1H NMR (400 MHz, CDCl₃): δ 8.46 (s, 1H), 8.10 (s, 1H), 4.38 (q, J=7.1 Hz, 2H), 4.15 (s, 2H), 3.63 (s, 3H), 1.35 (t, J=7.1 Hz, 3H)

### Step 3

Ethyl 2-bromo-4-(2-methoxy-2-oxo-ethyl)-5-nitro-benzoate (15.g, 43.3 mmol) was dissolved in acetic acid (60 mL) followed by the addition of iron (4.84 g, 86.7 mmol). The reaction was heated for 45 min at 100° C. The mixture was filtered over celite, while still warm, washing with EtOAc. The filtrate was concentrated in vacuo. Water was added to the residue and the solid was collected by vacuum filtration washing with water then minimal DCM (which removes the dark brown color and the solid goes light brown/golden) to afford ethyl 5-bromo-2-oxo-indoline-6-carboxylate (9.72 g, 34.2 mmol, 78.9% yield) as a golden solid.

UPLC-MS (ES⁺, Short acidic): 1.53 min, m/z 285.9 [M+H]⁺ (98%)

1H NMR (400 MHz, DMSO): δ 10.60 (s, 1H), 7.55 (s, 1H), 7.12 (s, 1H), 4.31 (q, J=7.1 Hz), 3.57 (s, 2H) 1.31 (t, J=7.1 Hz, 3H)

### Step 4

Ethyl 5-bromo-2-oxo-indoline-6-carboxylate (5.13 g, 18.1 mmol) and sodium hydride, (60% dispersed in mineral oil) (2.89 g, 72.2 mmol) were dissolved in dry THF (50 mL). The mixture was stirred for 30 minutes at 0° C. Iodomethane (4.5 mL, 72.2 mmol) was added and the RM was warmed slowly over two hours to RT. The reaction mixture was quenched with water. EtOAc was added and the two phases were separated. The aqueous reaction was re-extracted with EtOAc (2×). The combined organic extracts were filtered through phase separating filter paper and concentrated in vacuo to afford 5-bromo-1,3,3-trimethyl-2-oxo-indoline-6-carboxylic acid (5.30 g, 17.8 mmol, 98.5% yield) as a brown solid.

UPLC-MS (ES⁺, Short acidic): 1.39 min, m/z 298.0, 300.0 [M+H]⁺ (100%)

1H NMR (400 MHz, DMSO) 13.43 (s, 1H), 7.74 (s, 1H), 7.33 (s, 1H), 3.15 (s, 3H), 1.30 (s, 6H)

### Step 5

Sulfuric acid (1.48 mL, 26.7 mmol) was added to a stirred solution of 5-bromo-1,3,3-trimethyl-2-oxo-indoline-6-carboxylic acid (5.3 g, 17.8 mmol) and Ethanol (100 mL) at room temperature. The reaction was heated to 80° C. and stirred for 24 hours. The reaction was then cooled to room temperature and solvent removed in vacuo. The resulting mixture was poured onto an ice (˜500 mL)/NaHCO₃ mixture and then the resulting mixture extracted with EtOAc (2×300 mL). The combined organic layers were dried over sodium sulfate and solvent removed in vacuo. The crude material was purified by flash column chromatography (80 g, eluting in 0-80% EtOAc in pet ether) like fractions were pooled and concentrated in vacuo to afford to give ethyl 5-bromo-1,3,3-trimethyl-2-oxo-indoline-6-carboxylate (4.4 g, 13.5 mmol, 75.9% yield) as a yellow solid.

UPLC-MS (ES⁺, Short acidic): 1.85 min, m/z 326.1/328.0 [M+H]⁺; (100%)

1H-NMR (400 MHz, CDCl3): δ 7.44 (s, 1H), 7.21 (s, 1H), 4.43 (q, J=7.1 Hz, 2H), 3.23 (s, 3H), 1.43 (t, J=7.1 Hz, 3H), 1.38 (s, 6H).

### Step 6

To a stirring, thoroughly degassed solution of ethyl 5-bromo-1,3,3-trimethyl-2-oxo-indoline-6-carboxylate (4.5 g, 13.8 mmol), tributyl(1-ethoxyvinyl)tin (5.6 mL, 16.6 mmol) Triethylamine (4.8 mL, 34.5 mmol) in dry 1,4-Dioxane (90 mL) was added Bis(triphenylphosphine)palladium(II) dichloride (968 mg, 1.38 mmol). Reaction heated to 100° C. overnight.

The reaction mixture was cooled to rt and Hydrogen Chloride (conc.) (5.75 mL, 69.0 mmol) was added and stirred for 2 h. Water and EtOAc were added, and the layers were separated. Aq. extracted with EtOAc (×3). Combined organics washed with brine, dried over Na₂SO₄, filtered and concentrated. The crude was purified by column chromatography (80 g 0-100% EtOAc in pet ether) like fractions were pooled and concentrated in vacuo to afford ethyl 5-acetyl-1,3,3-trimethyl-2-oxo-indoline-6-carboxylate (3.99 g, 13.8 mmol, 99.96% yield) as a yellow solid.

UPLC-MS (ES⁺, Short acidic): 1.60 min, m/z 290.0 [M+H]⁺ (94%).

1H-NMR (400 MHz, CDCl₃): δ 7.35 (s, 1H), 7.19 (s, 1H), 4.40 (q, J=7.2 Hz, 2H), 3.26 (s, 3H), 2.55 (s, 3H), 1.40 (s, 6H), 1.39 (t, J=7.2 Hz, 3H).

### Step 7

To a stirring solution of ethyl 5-acetyl-1,3,3-trimethyl-2-oxo-indoline-6-carboxylate (3.9 g, 13.5 mmol) in Ethanol (50 mL) was added Hydrazine Hydrate (1.31 mL, 27.0 mmol). The reaction mixture was heated to 80° C. for 2 hours. A grey precipitate formed.

The reaction was concentrated under reduced pressure. Then the solid was filtered, washed with MTBE and dried in vacuum oven to afford 1,3,3,5-tetramethyl-7H-pyrrolo[3,2-g]phthalazine-2,8-dione (3.10 g, 12.1 mmol, 89.4% yield) as a grey solid.

UPLC-MS (ES⁺, Short acidic): 1.30 min, m/z 258.1 [M+H]⁺ (92%)

¹H-NMR (400 MHz, DMSO-d₆): δ 12.37 (s, 1H), 7.97 (s, 1H), 7.69 (s, 1H), 3.26 (s, 3H), 2.53 (s, 3H), 1.39 (s, 6H).

### Step 8

To 1,3,3,5-tetramethyl-7H-pyrrolo[3,2-g]phthalazine-2,8-dione (3.10 g, 12.1 mmol) was added phosphorus oxychloride (67.4 mL, 723 mmol) and dimethylaniline (3.05 mL, 24.1 mmol) and the reaction heated to 90° C. for 2.5 h.

Excess POCl₃ was removed under reduce pressure and quenched separately. The crude RM residue was quenched with sat. aq. NaHCO₃. The solid was filtered and dried under vacuum to give 8-chloro-1,3,3,5-tetramethyl-pyrrolo[3,2-g]phthalazin-2-one (2.68 g, 9.72 mmol, 80.7% yield) as a grey solid.

UPLC-MS (ES⁺, Short acidic): 1.40 min, m/z 276.1/278.0 [M+H]⁺ (100%)

1H NMR (400 MHz, DMSO-d6): δ 8.33 (s, 1H), 7.54 (s, 1H), 3.33 (s, 3H), 2.91 (s, 3H), 1.43 (s, 6H)

### 8-chloro-1,5-dimethyl-spiro[pyrrolo[3,2-g]phthalazine-3,4′-tetrahydropyran]-2-one (Int-22)

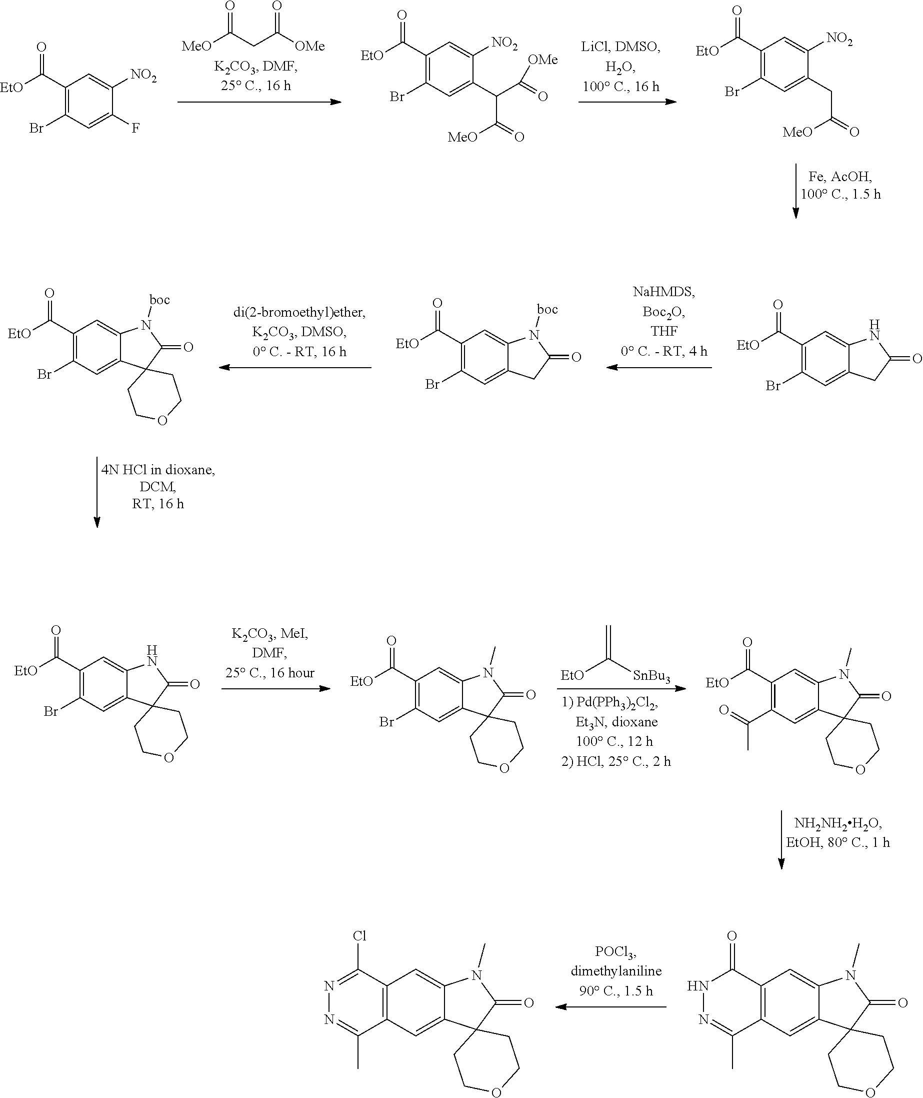

### Step 1

To a solution of Dimethylmalonate (1.18 mL, 10.3 mmol) in DMF (16 mL) at 0° C. was added ethyl 2-bromo-4-fluoro-5-nitro-benzoate (2.00 g, 6.85 mmol) and Potassium carbonate (2.84 g, 20.6 mmol). The reaction mixture was stirred at rt overnight. The reaction was poured into ice cold 1M aq. HCl and extracted with EtOAc (×3). Combined organic layers were washed with water, brine, dried over Na₂SO₄ and concentrated. The solid was washed with a small amount of MeOH and dried by vacuum filtration to afford dimethyl 2-(5-bromo-4-ethoxycarbonyl-2-nitro-phenyl)propanedioate (1.86 g, 4.60 mmol, 67% yield) as a yellow solid.

UPLC-MS (ES⁺, Short acidic): 1.85 min, m/z 404.0/405.9 [M+H]⁺ (97%).

1H-NMR (400 MHz, CDCl₃): δ 8.51 (s, 1H), 7.84 (s, 1H), 5.21 (s, 1H), 4.45 (q, J=7.1 Hz, 2H), 3.82 (s, 6H), 1.43 (t, J=7.2 Hz, 3H)

### Step 2

A solution of dimethyl 2-(5-bromo-4-ethoxycarbonyl-2-nitro-phenyl)propanedioate (1.86 g, 4.6 mmol) and Lithium chloride (390 mg, 9.20 mmol) in DMSO (18.5 mL) and Water (10.7 mL) was heated at 100° C. overnight. The reaction was cooled to rt and poured into ice-water. The solid was collected by vacuum filtration washing with water and dried to afford ethyl 2-bromo-4-(2-methoxy-2-oxo-ethyl)-5-nitro-benzoate (1.43 g, 4.12 mmol, 89.5% yield) as a pale yellow solid.

UPLC-MS (ES−, Short acidic): 1.86 min, m/z 344.2, 346.1 [M−H]⁻. (93%)

1H-NMR (400 MHz, CDCl₃): δ 8.58 (s, 1H), 7.70 (s, 1H), 4.45 (q, J=7.1 Hz, 2H), 4.04 (s, 2H), 3.72 (s, 3H), 1.44 (t, J=7.2 Hz, 3H).

### Step 3

To a solution of ethyl 2-bromo-4-(2-methoxy-2-oxo-ethyl)-5-nitro-benzoate (1.16 g, 3.34 mmol) in Acetic acid (11.6 mL) was added iron (1.12 g, 20.0 mmol). The reaction was heated at 100° C. for 45 min. The reaction mixture was filtered over celite, while still warm, washing with EtOAc. The filtrate was concentrated. The residue was taken up in water. The precipitate was collected by vacuum filtration washing with water then minimal DCM to afford ethyl 5-bromo-2-oxo-indoline-6-carboxylate (737 mg, 2.59 mmol, 77.7% yield) as a dark yellow solid.

UPLC-MS (ES⁺, Short acidic): 1.55 min, m/z 283.9/285. [M+H]⁻ ⁷⁹Br/⁸¹Br (98%).

¹H-NMR (400 MHz, DMSO-d₆) δ 10.60 (s, 1H), 7.56 (s, 1H), 7.13 (s, 1H), 4.31 (q, J=7.1 Hz, 2H), 3.58 (s, 2H), 1.32 (t, J=7.1 Hz, 3H).

### Step 4

To a solution of ethyl 5-bromo-2-oxo-indoline-6-carboxylate (5.19 g, 18.3 mmol) in dry THF (56 mL) at 0° C. was added dropwise Sodium bis(trimethylsilyl)amide 2M in THF (15.5 mL, 31.0 mmol). After 30 min at 0° C., Di-tert-butyl dicarbonate (6.38 g, 29.2 mmol) was added. The reaction mixture was stirred at 0° C. for 15 min and then warmed to RT for 3 hours. The reaction mixture was quenched with NH₄Cl (aq.). DCM was added and the two phases separated. The aqueous was extracted (3×) with DCM The combined organic extracts were washed with brine, dried over a phase separator, and concentrated in vacuo. The residue was purified by column chromatography (80 g, 0-50% EtOAc in petroleum ether), like fractions were pooled and concentrated to afford 01-tert-butyl 06-ethyl 5-bromo-2-oxo-indoline-1,6-dicarboxylate (3.31 g, 8.60 mmol, 47% yield) as a yellow solid.

LC-MS (ES−, Short acidic): 1.93 min, m/z 382.3/384.3 [M+H]⁻ (94%)

1H NMR (400 MHz, DMSO-d₆): δ 8.07 (s, 1H), 7.69 (s, 1H), 4.33 (q, J=7.1 Hz, 2H), 3.80 (s, 2H), 1.57 (s, 9H), 1.32 (t, J=7.1 Hz, 3H)

### Step 5

To a stirred solution of O1-tert-butyl 06-ethyl 5-bromo-2-oxo-indoline-1,6-dicarboxylate (1.0 g, 2.60 mmol) in DMSO (3.3 mL) at 0° C. was added Potassium carbonate (1.44 g, 10.4 mmol) and di(2-bromoethyl)ether (0.46 mL, 3.64 mmol). The reaction was stirred at 0° C. for 10 min and then warmed up to RT for 16 h. The reaction mixture was poured onto water followed by extraction with EtOAc (×2). The combined organic layers were washed with brine (×2), dried over Na₂SO₄, filtered and the solvent removed under reduce pressure. The crude material was purified by column chromatography (12 g, 0-40% EtOAc in petroleum ether) like fractions were pooled and concentrated in vacuo to afford 01-tert-butyl 06-ethyl 5-bromo-2-oxo-spiro[indoline-3,4′-tetrahydropyran]-1,6-dicarboxylate (899 mg, 1.98 mmol, 76% yield) as a yellow oil, which solidified upon standing.

UPLC-MS (ES⁺, Short acidic): 2.11 min, m/z 354.0/356.0 [M-Boc+H]⁺ (91%).

1H-NMR (400 MHz, CDCl₃): δ 8.27 (s, 1H), 7.56 (s, 1H), 4.42 (q, J=7.2 Hz, 2H), 4.27-4.19 (m, 2H), 3.93-3.86 (m, 2H), 1.98-1.90 (m, 2H), 1.88-1.81 (m, 2H), 1.66 (s, 9H), 1.41 (t, J=7.1 Hz, 3H)

### Step 6

To a stirred solution of O1-tert-butyl 06-ethyl 5-bromo-2-oxo-spiro[indoline-3,4′-tetrahydropyran]-1,6-dicarboxylate (899 mg, 1.98 mmol) in DCM (8.1 mL) was added hydrogen chloride 4N in dioxane (2.47 mL, 9.89 mmol) at 0° C. The reaction was stirred at RT overnight. The reaction was poured into water and extracted with EtOAc (×3). The combined organic layers were washed with water, dried over Na₂SO₄, filtered and concentrated in vacuo. The crude was purified by column (12 g, 0-10% MeOH in DCM) to give ethyl 5-bromo-2-oxo-spiro[indoline-3,4′-tetrahydropyran]-6-carboxylate (603 mg, 1.70 mmol, 86% yield) as a pale yellow solid.

UPLC-MS (ES⁺, Short acidic): 1.62 min, m/z 354.1/356.0 [M+H]⁺ (90%)

¹H-NMR (400 MHz, DMSO-d₆): δ 10.65 (s, 1H), 7.82 (s, 1H), 7.15 (s, 1H), 4.31 (q, J=7.1 Hz, 2H), 4.07-4.00 (m, 2H), 3.82-3.74 (m, 2H), 1.87-1.78 (m, 2H), 1.74-1.67 (m, 2H), 1.31 (t, J=7.1 Hz, 3H).

### Step 7

Ethyl 5-bromo-2-oxo-spiro[indoline-3,4′-tetrahydropyran]-6-carboxylate (603 mg, 1.70 mmol) and Potassium carbonate (294 mg, 2.13 mmol) were suspended in DMF (3.4 mL) and Iodomethane (0.12 mL, 1.87 mmol) was added. The reaction was stirred at rt overnight. Water was added and the reaction was extracted with EtOAc (×3). The combined organic layers were washed with brine (×2), dried over Na₂SO₄, filtered, and concentrated in vacuo. The crude was purified by column chromatography (12 g, 0-40% EtOAc in petroleum ether) like fractions were pooled and concentrated in vacuo to afford ethyl 5-bromo-1-methyl-2-oxo-spiro[indoline-3,4′-tetrahydropyran]-6-carboxylate (488 mg, 1.33 mmol, 77.8% yield) as a light brown solid.

UPLC-MS (ES⁺, Short acidic): 1.84 min, m/z 368.0/370.0 [M+H]⁺ (100%).

1H-NMR (400 MHz, CDCl₃): δ 7.60 (s, 1H), 7.21 (s, 1H), 4.43 (q, J=7.1 Hz, 2H), 4.29-4.21 (m, 2H), 3.94-3.86 (m, 2H), 3.22 (s, 3H), 1.87-1.81 (m, 4H), 1.43 (t, J=7.1 Hz, 3H).

### Step 8

To a solution of ethyl 5-bromo-1-methyl-2-oxo-spiro[indoline-3,4′-tetrahydropyran]-6-carboxylate (488 mg, 1.33 mmol), Tributyl(1-ethoxyvinyl)tin (0.54 mL, 1.59 mmol), Triethylamine (0.46 mL, 3.31 mmol) in 1,4-Dioxane (15 mL) was added Bis(triphenylphosphine)palladium(II) dichloride (93 mg, 0.13 mmol). The reaction mixture was heated to 100° C. overnight for 16 h. The reaction mixture was then cooled to RT and aq. 2M Hydrogen Chloride (3.31 mL, 6.63 mmol) was added and stirred for 1 hour. Water and EtOAc were added, and the layers separated. The aqueous was extracted with EtOAc (×3). Combined organics were washed with brine, dried over Na₂SO₄, filtered and concentrated. The crude material was purified by column chromatography (12 g, 0-100% EtOAc in petroleum ether) like fractions were pooled to afford ethyl 5-acetyl-1-methyl-2-oxo-spiro[indoline-3,4′-tetrahydropyran]-6-carboxylate (439 mg, 1.33 mmol, 100% yield) as an off-white solid.

UPLC-MS (ES⁺, Short acidic): 1.60 min, m/z 332.1 [M+H]⁺ (98%).

1H-NMR (400 MHz, CDCl₃): δ 7.48 (s, 1H), 7.18 (s, 1H), 4.40 (q, J=7.2 Hz, 2H), 4.31-4.23 (m, 2H), 3.95-3.88 (m, 2H), 3.25 (s, 3H), 2.55 (s, 3H), 1.96-1.87 (m, 2H), 1.86-1.77 (m, 2H), 1.39 (t, J=7.2 Hz, 3H).

### Step 9

To a stirring solution of ethyl 5-acetyl-1-methyl-2-oxo-spiro[indoline-3,4′-tetrahydropyran]-6-carboxylate (439 mg, 1.33 mmol) in Ethanol (8.3 mL) was added Hydrazine Hydrate (96.7 μL, 1.99 mmol). Reaction stirred at 80° C. for 3 h. The reaction mixture was concentrated in vacuo. The residue was then washed with MTBE, filtered, and dried to give 1,5-dimethylspiro[7H-pyrrolo[3,2-g]phthalazine-3,4′-tetrahydropyran]-2,8-dione (337 mg, 1.13 mmol, 85.0% yield) as a white solid.

UPLC-MS (ES⁺, Short acidic): 1.17 min, m/z 300.1 [M+H]⁺ (95%).

¹H-NMR (400 MHz, DMSO-d₆): δ 12.39 (s, 1H), 8.01 (s, 1H), 7.68 (s, 1H), 4.16-4.08 (m, 2H), 3.91-3.84 (m, 2H), 3.26 (s, 3H), 2.55 (s, 3H), 2.09-2.00 (m, 2H), 1.78-1.70 (m, 2H).

### Step 10

To 1,5-dimethylspiro[7H-pyrrolo[3,2-g]phthalazine-3,4′-tetrahydropyran]-2,8-dione (317.mg, 1.06 mmol) in phosphorus oxychloride (7.9 mL, 84.7 mmol) was added dimethylaniline (0.27 mL, 2.12 mmol) and the reaction heated to 90° C. for 1 h. POCl₃ was removed under reduce pressure. Sat. aq. NaHCO₃ was added till pH ˜7-8. The solid was filtered and dried in the vacuum oven to give 8-chloro-1,5-dimethyl-spiro[pyrrolo[3,2-g]phthalazine-3,4′-tetrahydropyran]-2-one (336.5 mg, 1.06 mmol, 100% yield) as a grey solid.

UPLC-MS (ES⁺, Short acidic): 1.28 min, m/z 318.1/320.1 [M+H]⁺ (100%).

1H-NMR (400 MHz, DMSO-d₆): 8.39 (s, 1H), 7.56 (s, 1H), 4.16-4.08 (m, 2H), 3.95-3.85 (m, 2H), 3.32 (s, 3H), 2.94 (s, 3H), 2.16-2.07 (m, 2H), 1.81-1.72 (m, 2H).

The following chlorophthalazine was prepared in a similar manner.

TABLE 3C
Intermediates Prepared According to the Disclosed Methods

| Compound | Structure | Analytical data |
|---|---|---|
| Int-23 | 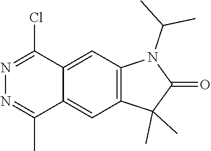 [SMILES: Cc1nnc(Cl)c2cc3c(cc12)C(C)(C)C(=O)N3C(C)C] | UPLC-MS (ES⁺, short acidic): 1.59 min, m/z 304.1/306.1 [M + H]⁺ (100%). |
| Int-24 | 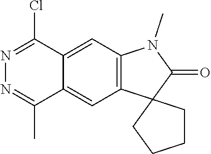 [SMILES: Cc1nnc(Cl)c2cc3c(cc12)C1(CCCC1)C(=O)N3C] | UPLC-MS (ES⁺, short acidic): 1.73 min, m/z 302.0/304.0 [M + H]+ (100%) |

### 8-chloro-1-cyclopropyl-3,3,5-trimethyl-pyrrolo[3,2-g]phthalazin-2-one (Int-25)

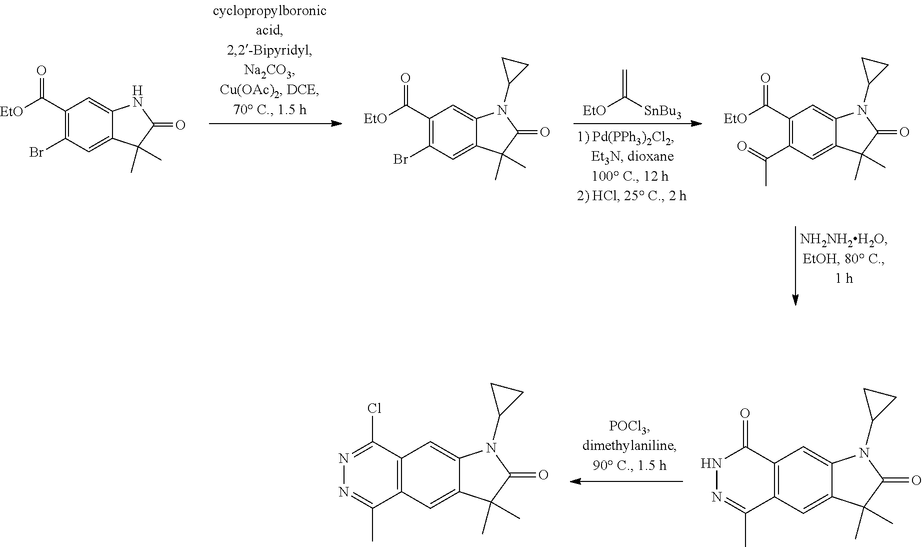

### Step 1

A mixture of ethyl 5-bromo-3,3-dimethyl-2-oxo-indoline-6-carboxylate (193.mg, 0.62 mmol), Copper(II) acetate (118 mg, 0.65 mmol), cyclopropylboronic acid (106.mg, 1.24 mmol), 2,2′-bipyridyl (101 mg, 0.65 mmol), sodium carbonate (144 mg, 1.36 mmol) in DCE (5.8 mL) was bubbled with air and stirred at 70° C. for 2 h. The reaction was quenched with sat. aq. NH₄Cl, extracted with DCM (×3). Organic layers were combined, washed with brine, dried over Na₂SO₄, filtered and concentrated in vacuo. The crude product was purified by column chromatography using as eluent a gradient 0-50% EtOAc in petroleum ether affording ethyl 5-bromo-1-cyclopropyl-3,3-dimethyl-2-oxo-indoline-6-carboxylate (241 mg, 0.45 mmol, 73.1% yield) as a pale yellow oil.

UPLC-MS (ES⁺, short acidic): 1.89 min, m/z 352.1/354.0 [M+H]⁺ (67%).

1H-NMR (400 MHz, CDCl₃): 7.43 (s, 1H), 7.41 (s, 1H), 4.44 (q, J=7.1 Hz, 2H), 2.70-2.62 (m, 1H), 1.43 (t, J=7.1 Hz, 3H), 1.34 (s, 6H), 1.12-1.05 (m, 2H), 0.93-0.87 (m, 2H)

### Step 2

Made in the same way as 8-chloro-1,5-dimethyl-spiro[pyrrolo[3,2-g]phthalazine-3,4′-tetrahydropyran]-2-one Step 8. Ethyl 5-acetyl-1-cyclopropyl-3,3-dimethyl-2-oxo-indoline-6-carboxylate (quantitative yield) as a pale yellow solid.

UPLC-MS (ES⁺, Short acidic): 1.77 min, m/z 316.1 [M+H]⁺ (89%).

1H-NMR (400 MHz, CDCl3):7.42 (s, 1H), 7.33 (s, 1H), 4.40 (q, J=7.1 Hz, 2H), 2.71-2.66 (m, 1H), 2.54 (s, 3H), 1.39 (t, J=7.1 Hz, 3H), 1.36 (s, 6H), 1.15-1.09 (m, 2H), 0.95-0.88 (m, 2H)

### Step 3

Made in the same way as 8-chloro-1,5-dimethyl-spiro[pyrrolo[3,2-g]phthalazine-3,4′-tetrahydropyran]-2-one Step 9. 1-cyclopropyl-3,3,5-trimethyl-7H-pyrrolo[3,2-g]phthalazine-2,8-dione (117 mg, 0.41 mmol, 57.9% yield) as an off-white solid.

UPLC-MS (ES⁺, Short acidic): 1.42 min, m/z 284.0 [M+H]⁺ (100%).

¹H-NMR (400 MHz, DMSO-d₆): 12.36 (s, 1H), 7.94 (s, 1H), 7.79 (s, 1H), 2.88-2.80 (m, 1H), 2.52 (s, 3H), 1.35 (s, 6H), 1.11-1.05 (m, 2H), 0.86-0.81 (m, 2H)

### Step 4

Made in the same way as 8-chloro-1,5-dimethyl-spiro[pyrrolo[3,2-g]phthalazine-3,4′-tetrahydropyran]-2-one Step 9 8-chloro-1-cyclopropyl-3,3,5-trimethyl-pyrrolo[3,2-g]phthalazin-2-one (180 mg, 0.40 mmol, 96.8% yield) (purity 67%—in mixture with dimethylaniline.

UPLC-MS (ES⁺, short acidic): 1.47 min, m/z 302.1/304.0 [M+H]⁺ (100%).

¹H-NMR (400 MHz, DMSO-d₆): 8.32 (s, 1H), 7.63 (s, 1H), 2.91 (s, 3H), 2.91-2.87 (m, 1H), 1.40 (s, 6H), 1.16-1.10 (m, 2H), 0.91-0.85 (m, 2H).

The following chlorophthalazine was prepared in a similar manner.

TABLE 3D
Intermediates Prepared According to the Disclosed Methods

| Compound | Structure | Analytical data |
|---|---|---|
| Int-26 | 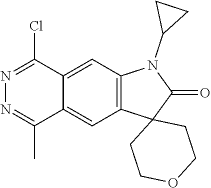 [SMILES: Cc1nnc(Cl)c2cc3c(cc12)C1(CCOCC1)C(=O)N3C1CC1] | UPLC-MS (ES⁺, short acidic, UPLCMS- SQD): 1.40 min, m/z 344.2/346.1 [M + H]⁺ (100%). |

### 8-chloro-3-(methoxymethyl)-1,3,5-trimethyl-pyrrolo[3,2-g]phthalazine-2-one (Int-27)

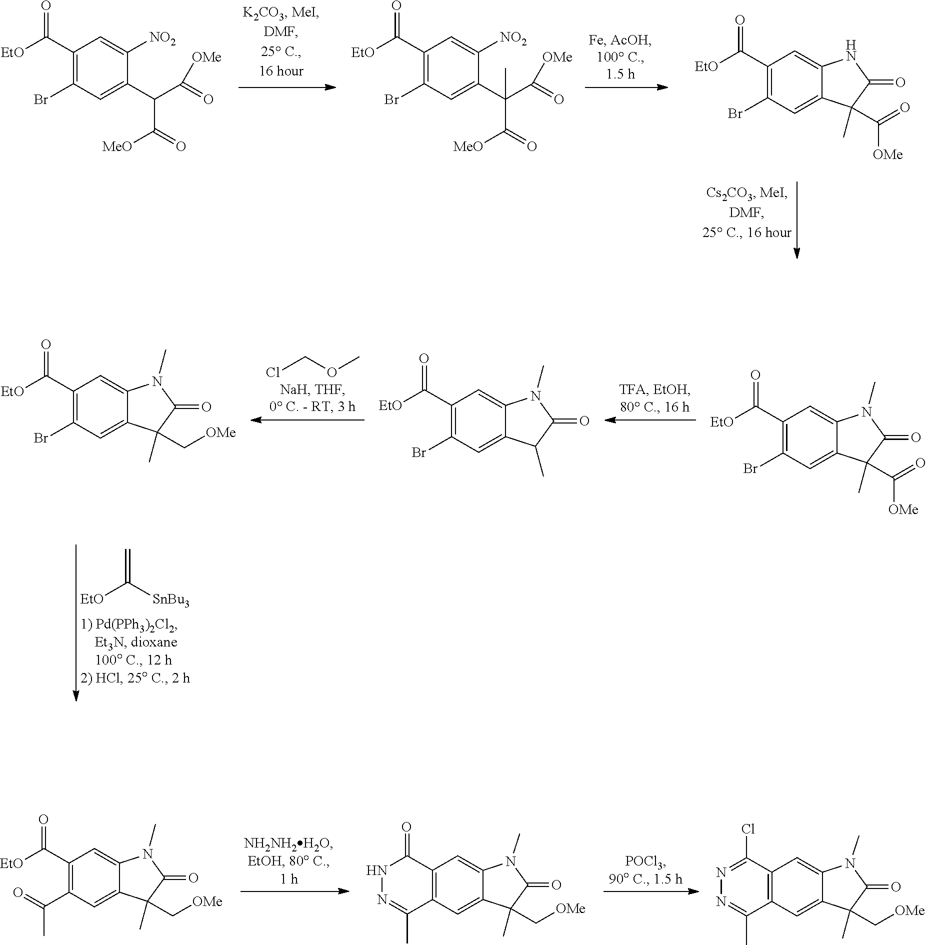

### Step 1

To a stirred solution of dimethyl 2-(5-bromo-4-ethoxycarbonyl-2-nitro-phenyl)propanedioate (4.87 g, 12.1 mmol) in DMF (15 mL) was added potassium carbonate (2.33 g, 16.9 mmol) and iodomethane (1.05 mL, 16.9 mmol) the reaction was stirred at RT overnight. The mixture was diluted with water and EtOAc and the two phases were separated. The aqueous was extracted with EtOAc (2×). The combined organic extracts were washed with Brine, passed through phase separating filter paper and concentrated in vacuo to afford dimethyl 2-(5-bromo-4-ethoxycarbonyl-2-nitro-phenyl)-2-methyl-propanedioate (4.86 g, 11.6 mmol, 96.4% yield) as a yellow solid.

UPLC-MS (ES⁺, Short acidic): 2.04 min, m/z 418.0 [M+H]⁺ (100%)

1H NMR (400 MHz, CDCl₃): 8.51 (s, 1H), 7.67 (s, 1H), 4.45 (q, J=7.1 Hz, 2H), 3.75 (s, 6H), 2.02 (s, 3H), 1.43 (t, J=7.1 Hz, 3H).

### Step 2

To a suspension of dimethyl 2-(5-bromo-4-ethoxycarbonyl-2-nitro-phenyl)-2-methyl-propanedioate (5.28 g, 12.6 mmol) in Acetic acid (49.9 mL) and Ethanol (49.9 mL) was added iron (1.94 g, 34.7 mmol). The RM was stirred at 100° C. for 1 hr. The reaction mixture was filtered through Celite and concentrated in vacuo. The crude material was partitioned between water and EtOAc the two phases were separated. The aqueous was extracted with EtOAc (3×). The combined organics were washed with brine, passed through hydrophobic filter paper and concentrated in vacuo to afford 06-ethyl O3-methyl 5-bromo-3-methyl-2-oxo-indoline-3,6-dicarboxylate (2.10 g, 5.90 mmol, 46.7% yield) as a purple solid.

UPLC-MS (ES⁺, Short acidic): 1.81 min, m/z 358.0 [M+H]⁺ (100%)

1H NMR (400 MHz, CDCl₃): 7.51 (s, 1H), 7.37 (br.s, 1H), 7.27 (s, 1H), 4.25 (q, J=7.0 Hz, 2H), 3.72 (s, 3H), 1.71 (s, 3H), 1.41 (t, J=7.0 Hz, 3H)

### Step 3

To a suspension of 06-ethyl O3-methyl 5-bromo-3-methyl-2-oxo-indoline-3,6-dicarboxylate (2.07 g, 5.81 mmol), cesium carbonate (3.79 g, 11.6 mmol) in DMF (15 mL) was added Iodomethane (0.47 mL, 7.56 mmol). The RM was stirred for 3 hrs at RT. Water and EtOAc added, the two phases were separated and the aqueous was extracted with EtOAc (×2). The combined organics were washed with water, brine, passed through hydrophobic filter paper and concentrated in vacuo to afford 06-ethyl O3-methyl 5-bromo-1,3-dimethyl-2-oxo-indoline-3,6-dicarboxylate (1.97 g, 5.32 mmol, 91.6% yield) as a red solid.

UPLC-MS (ES⁺, Short acidic): 1.91 min, m/z 370.0 372.0 [M+H]⁺ (100%)

1H NMR (400 MHz, CDCl₃) 7.50 (s, 1H), 7.22 (s, 1H), 4.43 (q, J=7.1 Hz, 2H), 3.68 (s, 3H), 3.27 (s, 3H), 1.67 (s, 3H), 1.43 (t, J=7.1 Hz, 3H)

### Step 4

O6-ethyl O3-methyl 5-bromo-1,3-dimethyl-2-oxo-indoline-3,6-dicarboxylate (1.36 g, 3.67 mmol) was suspended in Sulfuric acid (1.96 mL, 36.7 mmol) and Trifluoroacetic acid (2.81 mL, 36.7 mmol). The RM was heated at 60° C. for 16 hours. After cooling to RT, the TFA is removed in vacuo. The crude residue was taken up in Ethanol (20 mL) and heated at 80° C. for 16 hours. After cooling to rt the RM was concentrated in vacuo. The residue taken up in EtOAc, and basified to pH-8 using ice cold, sat. aq. NaHCO₃. The two phases were separated and the aqueous was extracted with EtOAc (2×). The combined organics were washed with brine, passed through hydrophobic filter paper and concentrated in vacuo. The crude material was purified by flash chromatography (25 g, 0-100% EtOAc in pet ether) like fractions were pooled and concentrated in vacuo to afford ethyl 5-bromo-1,3-dimethyl-2-oxo-indoline-6-carboxylate (763 mg, 2.44 mmol, 66.5% yield) as a yellow solid.

UPLC-MS (ES⁺, Short acidic): 1.83 min, m/z 312.0 314.0 [M+H]⁺ (93%)

1H NMR (400 MHz, CDCl₃) 7.49 (d, J=1.0 Hz, 1H), 7.19 (s, 1H), 4.43 (q, J=7.1 Hz, 2H), 3.50-3.43 (m, 1H), 3.22 (s, 3H), 1.48 (d, J=7.6 Hz, 3H), 1.43 (t, J=7.1 Hz, 3H)

### Step 5

Sodium hydride, (60% dispersed in mineral oil) (117 mg, 2.93 mmol) was added to a suspension of ethyl 5-bromo-1,3-dimethyl-2-oxo-indoline-6-carboxylate (763 mg, 2.44 mmol) in THF (20 mL) at 0° C. After 30 mins, chloromethyl methyl ether (278 μL, 3.67 mmol) was added.

The RM was warmed slowly to RT and stirred for 16 hours. The RM was cooled to 0° C. and Sodium hydride, (60% dispersed in mineral oil) (57 mg, 1.47 mmol) was added. After 30 mins Chloromethyl methyl ether (0.37 mL, 4.89 mmol) was added. The RM was stirred for 24 hrs.

The RM was quenched with water at RT. The aqueous was extracted with EtOAc (3×). Combined organics were washed with brine, passed through hydrophobic filter paper and concentrated in vacuo. The crude material was purified by flash chromatography (25 g, 10-40% EtOAc in Pet ether) like fractions were pooled and concentrated in vacuo to afford ethyl 5-bromo-3-(methoxymethyl)-1,3-dimethyl-2-oxo-indoline-6-carboxylate (681 mg, 1.91 mmol, 78.2% yield) as a yellow solid.

UPLC-MS (ES⁺, Short acidic): 1.86 min, m/z 356.0 [M+H]⁺ (78%)

1H NMR (400 MHz, CDCl₃) 7.50 (s, 1H), 7.20 (s, 1H), 4.42 (q, J=7.1 Hz, 2H), 3.61 (s, 2H), 3.23-3.22 (m, 6H), 1.42 (t, J=7.1 Hz, 3H), 1.33 (s, 3H)

### Step 6

A solution of ethyl 5-bromo-3-(methoxymethyl)-1,3-dimethyl-2-oxo-indoline-6-carboxylate (681 mg, 1.91 mmol), tributyl(1-ethoxyvinyl)tin (0.97 mL, 2.87 mmol), Triethylamine (0.53 mL, 3.82 mmol) in 1,4-Dioxane (15 mL) was degassed with N₂ for 5 mins. Bis(triphenylphosphine)palladium(II) dichloride (134 mg, 0.19 mmol) was added. The RM was heated at 100° C. for 5 hrs. After cooling to rt, hydrogen chloride (2N aqueous solution) (4.78 mL, 9.56 mmol) was added, and reaction was stirred RT for 16 hours. The RM was partitioned between water and EtOAc. The two phases were separated and the aqueous was extracted with EtOAc (2×). The combined organics were washed with brine, passed through hydrophobic filter paper and concentrated in vacuo. The crude material was purified by flash column chromatography (25 g, 20-60% EtOAc in Pet Ether) like fractions were pooled and concentrated in vacuo to afford ethyl 5-acetyl-3-(methoxymethyl)-1,3-dimethyl-2-oxo-indoline-6-carboxylate (455 mg, 1.42 mmol, 74.5% yield) as a colourless oil.

UPLC-MS (ES⁺, Short acidic): 1.58 min, m/z 320.1 [M+H]⁺ (84%)

1H NMR (400 MHz, CDCl₃,) 7.42 (s, 1H), 7.16 (s, 1H), 4.39 (q, J=7.1 Hz, 2H), 3.63 (q, J=8.2 Hz, 2H), 3.26 (s, 3H), 3.23 (s, 3H), 2.55 (s, 3H), 1.38 (t, J=7.1 Hz, 3H), 1.35 (s, 3H)

### Step 7

To a solution of ethyl 5-acetyl-3-(methoxymethyl)-1,3-dimethyl-2-oxo-indoline-6-carboxylate (455 mg, 1.42 mmol) in Ethanol (15 mL) was added Hydrazine Hydrate (0.14 mL, 2.85 mmol). The RM was heated at 80° C. for 16 hours. The RM was concentrated in vacuo. The residue was suspended in MTBE and filtered to afford 3-(methoxymethyl)-1,3,5-trimethyl-7H-pyrrolo[3,2-g]phthalazine-2,8-dione (355 mg, 1.24 mmol, 86.7% yield) as a yellow solid.

UPLC-MS (ES⁺, Short acidic): 1.39 min, m/z 288.0 [M+H]⁺ (88%)

1H NMR (400 MHz, DMSO-d₆): 12.38 (s, 1H), 7.97 (s, 1H), 7.67 (s, 1H), 3.85 (d, J=8.9 Hz, 1H), 3.67 (d, J=8.9 Hz, 1H), 3.25 (s, 3H), 3.07 (s, 3H), 2.53 (s, 3H), 1.30 (s, 3H)

### Step 8

A suspension of 3-(methoxymethyl)-1,3,5-trimethyl-7H-pyrrolo[3,2-g]phthalazine-2,8-dione (115 mg, 0.40 mmol) in phosphorus oxychloride (0.75 mL, 8.01 mmol) was heated at 90° C. for 90 minutes. The reaction mixture was concentrated in vacuo. To the residue was added a small amount ice-cold water. This was added to ice-cold NaHCO3 (sat. aq.) so that pH-8. The solid was collected by vacuum filtration and dried to afford 8-chloro-3-(methoxymethyl)-1,3,5-trimethyl-pyrrolo[3,2-g]139hthalazine-2-one (101 mg, 0.33 mmol, 82.5% yield) as a yellow solid.

UPLC-MS (ES⁺, Short acidic): 1.45 min, m/z 306.0 [M+H]⁺ (92%)

1H NMR (400 MHz, CDCl₃), 7.86 (s, 1H), 7.49 (s, 1H), 3.74 (q, J=9.7 Hz, 2H), 3.40 (s, 3H), 3.23 (s, 3H), 2.98 (s, 3H), 1.46 (s, 3H)

### 8-chloro-1-cyclopropyl-3-(methoxymethyl)-3,5-dimethyl-1H, 2H, 3H-pyrrolo[2,3-g]phthalazin-2-one (Int-28)

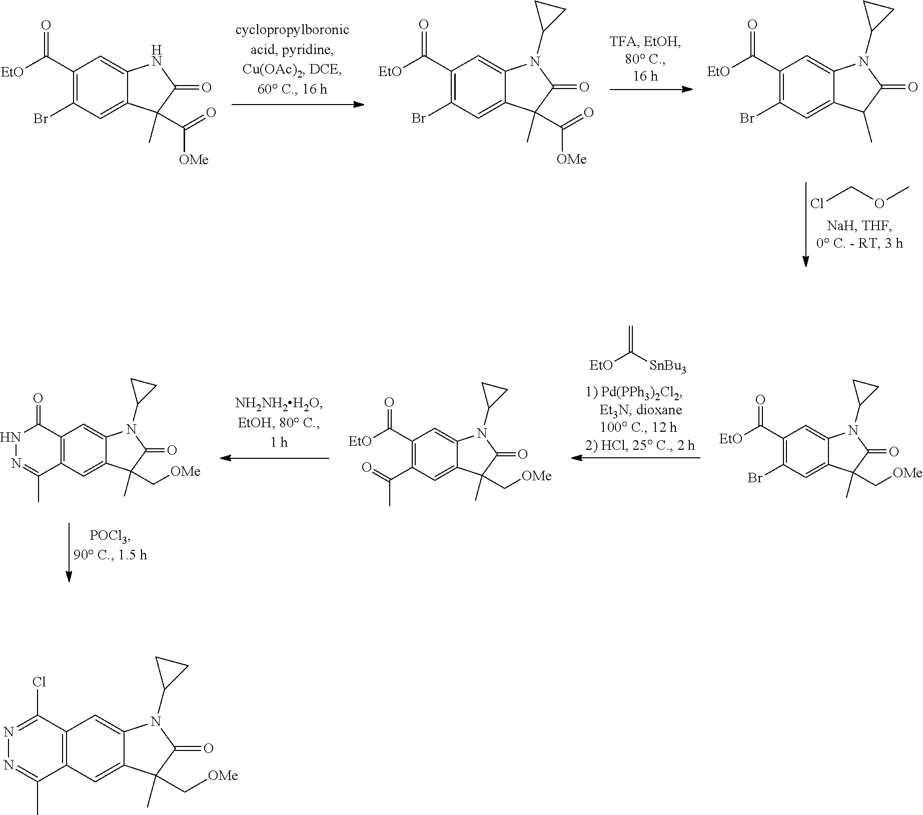

### Step 1

To a suspension of diethyl 5-bromo-3-methyl-2-oxo-indoline-3,6-dicarboxylate (4.g, 10.8 mmol) in DCE (40 mL) is added cyclopropylboronic acid (1.86 g, 21.6 mmol) Pyridine (0.87 mL, 10.8 mmol) sodium carbonate (2.52 g, 23.8 mmol) Copper(II) acetate (2.06 g, 11.4 mmol). Mixed in DCE (40 mL) at 60° C. for 20 h. Water and DCM added, and the mix filtered. Layers separated, and aq. extracted ×3 with DCM. Combined organics washed with brine, passed through hydrophobic frit and concentrated in vacuo. Crude residue purified by flash chromatography (40 g, dry-load, 10-60% EtOAc in Petroleum Ether) to yield diethyl 5-bromo-1-cyclopropyl-3-methyl-2-oxo-indoline-3,6-dicarboxylate (1.3 g, 3.17 mmol, 29.3% yield) as a clear oil.

UPLC-MS (ES⁺, Short acidic): 1.99 min, m/z 412.0 [M+H]⁺ (81%)

1H NMR (400 MHz, CDCl3): 7.49 (s, 1H), 7.42 (s, 1H), 4.44 (q, J=7.1 Hz, 2H), 4.21-4.08 (m, 2H), 2.74-2.67 (m, 1H), 1.63 (s, 3H), 1.47-1.41 (m, 3H), 1.19-1.07 (m, 5H), 1.00-0.83 (m 2H)

### Step 2

Made in the same way as 8-chloro-3-(methoxymethyl)-1,3,5-trimethyl-pyrrolo[3,2-g]140hthalazine-2-one Step 4. Ethyl 5-bromo-1-cyclopropyl-3-methyl-2-oxo-indoline-6-carboxylate (590 mg, 1.74 mmol, 27.2% yield) as a yellow oil

UPLC-MS (ES⁺, Short acidic): 1.95 min, m/z 338.0 [M+H]⁺ (61%)

1H NMR (400 MHz, CDCl3, AD-N203650-20-01_1HNMR): 7.45 (s, 1H), 7.41 (s, 1H), 4.43 (q, J=7.1 Hz, 2H), 3.40 (q, J=7.47, 1H), 2.68-2.60 (m, 1H), 1.48-1.40 (m, 6H), 1.13-1.04 (m, 2H), 0.98-0.84 (m, 2H)

### Step 3

Made in the same way as 8-chloro-3-(methoxymethyl)-1,3,5-trimethyl-pyrrolo[3,2-g]140hthalazine-2-one Step 5. ethyl 5-bromo-1-cyclopropyl-3-(methoxymethyl)-3-methyl-2-oxo-indoline-6-carboxylate (582 mg, 1.52 mmol, 87.3% yield) as a yellow oil.

UPLC-MS (ES⁺, Short acidic): 1.96 min, m/z 384.0 [M+H]⁺ (79%)

### Step 4

Made in the same way as 8-chloro-3-(methoxymethyl)-1,3,5-trimethyl-pyrrolo[3,2-g]140hthalazine-2-one Step 6. ethyl 5-acetyl-1-cyclopropyl-3-(methoxymethyl)-3-methyl-2-oxo-indoline-6-carboxylate (224 mg, 0.65 mmol, 42.6% yield) as a clear oil.

UPLC-MS (ES⁺, Short acidic): 1.71 min, m/z 346.1.1 [M+H]⁺ (81%)

### Step 5

Made in the same way as 8-chloro-3-(methoxymethyl)-1,3,5-trimethyl-pyrrolo[3,2-g]140hthalazine-2-one Step 7.

1-cyclopropyl-3-(methoxymethyl)-3,5-dimethyl-7H-pyrrolo[3,2-g]phthalazine-2,8-dione (161 mg, 0.51 mmol, 79.2% yield) as a white solid.

UPLC-MS (ES⁺, Short acidic): 1.39 min, m/z 314.1 [M+H]⁺ (77%)

1H NMR (400 MHz, DMSO-d₆): 12.41 (s, 1H), 7.94 (s, 1H), 7.77 (s, 1H), 3.81 (d, J=9.0 Hz, 1H), 3.61 (d, J=9.0 Hz, 1H), 3.06 (s, 3H), 2.86-2.80 (m, 1H), 1.27 (s, 3H), 1.12-1.06 (m, 2H), 0.82-0.76 (m, 2H). CH₃ group under the DMSO solvent peak.

### Step 6

Made in the same way as 8-chloro-3-(methoxymethyl)-1,3,5-trimethyl-pyrrolo[3,2-g]141hthalazine-2-one Step 8. 8-chloro-3-(methoxymethyl)-1,3,5-trimethyl-pyrrolo[3,2-g]phthalazin-2-one (321 mg, 1.05 mmol, 86.2% yield) as a yellow/off-white solid

UPLC-MS (ES⁺, Short acidic): 1.55 min, m/z 332.1 [M+H]⁺ (67%)

### 8-chloro-3-methoxy-1,3,5-trimethyl-pyrrolo[3,2-g]phthalazin-2-one (Int-29)

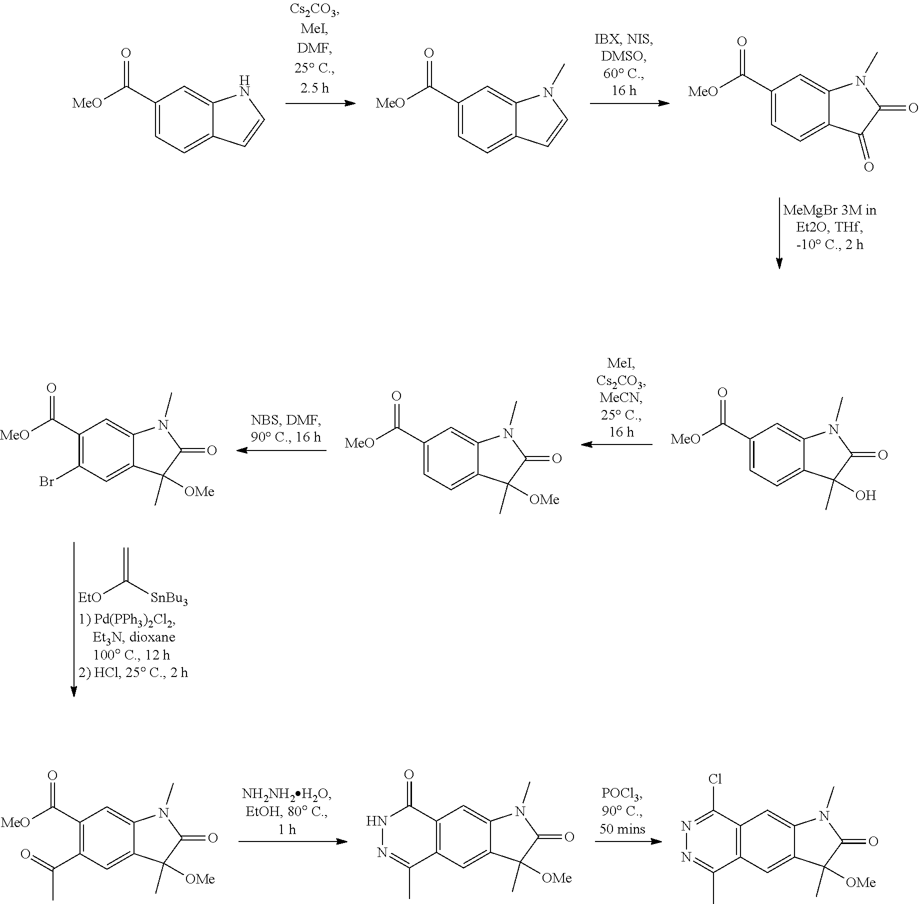

### Step 1

To a stirring suspension of Methyl 1H-indole-6-carboxylate (500 mg, 2.85 mmol) and cesium carbonate (1.39 g, 4.28 mmol) in DMF (3 mL) was added iodomethane (355 μL, 5.71 mmol). The mixture was stirred at 25° C. for 2.5 hours. Ice was added and the resulting precipitate was filtered, washed with cold water and dried in the vacuum oven to afford methyl 1-methylindole-6-carboxylate (481 mg, 2.54 mmol, 89.1% yield) as an off-white solid.

UPLC-MS (ES⁺, Short acidic): 1.80 min, m/z 190.0 [M+H]⁺ (100%) ¹H NMR (400 MHz, DMSO-d₆): 8.11-8.08 (m, 1H), 7.67-7.61 (m, 2H), 7.57 (d, J=3.0 Hz, 1H), 6.52 (dd, J=0.9, 3.0 Hz, 1H), 3.87 (s, 3H), 3.86 (s, 3H)

### Step 2

To a stirring solution of methyl 1-methylindole-6-carboxylate (2.16 g, 11.4 mmol) in DMSO (16 mL) was added N-iodosuccinimide (3.85 g, 17.1 mmol) followed by IBX (17.8 g, 28.5 mmol). The reaction was heated to 60° C. overnight. The reaction was partitioned between DCM and water, then filtered. The two phases were separated and the aqueous layer was extracted with DCM (3×). The combined organic extracts were washed with brine 3×, passed through a phase separator and concentrated under reduced pressure. The residue was then purified by flash column chromatography twice (40 g, eluting with 20-100% EtOAc in PET then 0-20% MeOH in DCM) to afford methyl 1-methyl-2,3-dioxo-indoline-6-carboxylate (1.80 g, 8.21 mmol, 71.9% yield) as an orange solid.

UPLC-MS (ES⁺, Short acidic): 1.42 min, m/z 219.9 [M+H]⁺ (100%)

1H NMR (400 MHz, DMSO-d₆): δ 7.73-7.63 (m, 2H), 7.57-7.54 (m, 1H), 3.91 (s, 3H), 3.20 (s, 3H)

### Step 3

To a suspension of methyl 1-methyl-2,3-dioxo-indoline-6-carboxylate (1.83 g, 8.35 mmol) in THF (30 mL) under nitrogen at −10° C. was added bromo(methyl)magnesium 3M in diethyl ether (5.6 mL, 16.7 mmol). After 2 hours, the reaction was quenched with a sat. sol. of citric acid, extracted with EtOAc (2×). The combined organic extracts were dried over MgSO4 and concentrated. The residue was then purified by flash column chromatography (40 g, eluting with 20-100% EtOAc in petroleum ether) to afford methyl 3-hydroxy-1,3-dimethyl-2-oxo-indoline-6-carboxylate (1.77 g, 7.52 mmol, 90.1% yield) as a green gum.

UPLC-MS (ES⁺, Short acidic): 1.25 min, m/z 236.0 [M+H]⁺ (90%)

1H NMR (400 MHz, DMSO-d₆): δ 7.71 (dd, J=1.4, 7.6 Hz, 1H), 7.48 (d, J=7.6 Hz, 1H), 7.47 (d, J=1.2 Hz, 1H), 6.11 (s, 1H), 3.87 (s, 3H), 3.15 (s, 3H), 1.40 (s, 3H)

### Step 4

To a suspension of methyl 3-hydroxy-1,3-dimethyl-2-oxo-indoline-6-carboxylate (2.00 g, 8.50 mmol) in MeCN (20 mL) was added cesium carbonate (11.0 g, 33.8 mmol) followed by iodomethane (2.65 mL, 42.5 mmol) at 25° C. The reaction was stirred overnight. The reaction was partitioned between DCM and water. The aqueous layer was extracted with DCM 3×. The organic phase was passed through a phase separator and concentrated under reduced pressure. The residue was purified by flash column chromatography (40 g, eluting with 0-100% EtOAc in petroleum ether) to afford methyl 3-methoxy-1,3-dimethyl-2-oxo-indoline-6-carboxylate (1.02 g, 4.09 mmol, 48.1% yield) as a green solid.

UPLC-MS (ES⁺, Short acidic): 1.57 min, m/z 250.0 [M+H]⁺ (100%)

1H NMR (400 MHz, DMSO-d₆): δ7.84 (dd, J=1.4, 7.6 Hz, 1H), 7.50 (d, J=1.1 Hz, 1H), 7.39 (dd, J=0.4, 7.6 Hz, 1H), 3.95 (s, 3H), 3.27 (s, 3H), 3.02 (s, 3H), 1.56 (s, 3H)

### Step 5

A stirring solution of methyl 3-methoxy-1,3-dimethyl-2-oxo-indoline-6-carboxylate (852 mg, 3.42 mmol) and N-bromosuccinimide (1.22 g, 6.84 mmol) in DMF (3 mL) was heated to 90° C. overnight. The reaction was partitioned between DCM and water. The two phases were separated, and the aqueous layer was extracted with DCM (3×). The combined organic extracts were washed with brine 2×, passed through a phase separator and concentrated under reduced pressure. The residue was then purified by flash column chromatography (25 g, eluting with 0-100% EtOAc in petroleum ether) to afford methyl 5-bromo-3-methoxy-1,3-dimethyl-2-oxo-indoline-6-carboxylate (855 mg, 2.61 mmol, 76.2% yield) as a pale yellow solid.

UPLC-MS (ES⁺, Short acidic): 1.70 min, m/z 328.0/330.0 [M+H]⁺ (100%)

1H NMR (400 MHz, DMSO-d₆): δ 7.58 (s, 1H), 7.22 (s, 1H), 3.97 (s, 3H), 3.23 (s, 3H), 3.04 (s, 3H), 1.55 (s, 3H)

### Step 6

To a thoroughly degassed solution of ethyl 6-bromo-1,3-dimethyl-2-oxo-benzimidazole-5-carboxylate (7.76 g, 24.8 mmol), Tributyl(1-ethoxyvinyl)tin (9.2 mL, 27.3 mmol) and triethylamine (8.7 mL, 62.7 mmol) in 1,4-Dioxane (20 mL) was added Bis(triphenylphosphine)palladium(II) dichloride (1.74 g, 2.48 mmol). The mixture was degassed for an additional 10 min, then the reaction heated to 90° C. for 2 hours. The reaction was cooled with an ice-water bath and conc. hydrogen chloride (6.2 mL, 74.3 mmol) was slowly added. The reaction was stirred for 30 min. The mixture was taken up in H₂O and DCM. The two phases were separated and the aqueous phase was extracted with DCM (3×). The combined organic extracts were washed with brine, passed through a phase separator and concentrated in vacuo. The crude residue was purified by flash column chromatography (25 g, eluting with 0-100% EtOAc in petroleum ether) to afford methyl 5-acetyl-3-methoxy-1,3-dimethyl-2-oxo-indoline-6-carboxylate (727 mg, 2.50 mmol, 92.5% yield) as a beige solid.

UPLC-MS (ES⁺, Short acidic): 1.44 min, m/z 292.0 [M+H]⁺ (100%)

1H NMR (400 MHz, CDCl₃): δ 7.54 (s, 1H), 7.15 (s, 1H), 3.93 (s, 3H), 3.27 (s, 3H), 3.04 (s, 3H). 2.57 (s, 3H), 1.57 (s, 3H)

### Step 7

To a stirring solution of methyl 5-acetyl-3-methoxy-1,3-dimethyl-2-oxo-indoline-6-carboxylate (720 mg, 2.47 mmol) in Ethanol (10 mL) was added hydrazine hydrate (240 μL, 4.94 mmol). The reaction mixture was heated to 80° C. for 3 hours then overnight at room temperature. The reaction was concentrated under reduced pressure, stirred with a minimal amount of cold EtOH in an ice-water bath. The solid was filtered, washed with EtOH then MTBE and dried in vacuum oven to afford 3-methoxy-1,3,5-trimethyl-7H-pyrrolo[3,2-g]phthalazine-2,8-dione (522 mg, 1.91 mmol, 77.3% yield) as a pale grey solid.

UPLC-MS (ES⁺, Short acidic): 1.25 min, m/z 274.0 [M+H]⁺ (100%)

1H NMR (400 MHz, DMSO-d₆): δ 12.46 (s, 1H), 7.92 (s, 1H), 7.73 (s, 1H), 3.28 (s, 3H), 2.92 (s, 3H), 2.54 (s, 3H), 1.54 (s, 3H)

### Step 8

A stirring suspension of 3-methoxy-1,3,5-trimethyl-7H-pyrrolo[3,2-g]phthalazine-2,8-dione (910 mg, 3.33 mmol) in phosphorus oxychloride (4.00 mL, 42.9 mmol) was heated to 90° C. for 50 minutes. POCl₃ was removed under reduce pressure. The reaction was cooled down to 0° C. and basified with an ice-cold saturated solution of Na2CO₃ (pH 7). The solid was collected by vacuum filtration, washed with water and dried to afford 8-chloro-3-methoxy-1,3,5-trimethyl-pyrrolo[3,2-g]phthalazin-2-one (930 mg, 3.19 mmol, 95.7% yield) as a pale grey solid.

UPLC-MS (ES⁺, short acidic): 1.39 min, m/z 292.0/294.0 [M+H]⁺ (100%)

The following chlorophthalazine was prepared in a similar manner.

TABLE 3E
Intermediates Prepared According to the Disclosed Methods

| Compound | Structure | Analytical data |
|---|---|---|
| Int-30 | 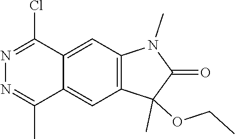 [SMILES: CCOC1(C)C(=O)N(C)c2cc3c(Cl)nnc(C)c3cc21] | UPLC-MS (ES⁺, Short acidic,): 1.51 min, m/z 306.0/308.0 [M + H]⁺ (95%) |
| Int-31 | 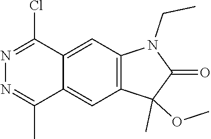 [SMILES: CCN1C(=O)C(C)(OC)c2cc3c(C)nnc(Cl)c3cc21] | UPLC-MS (ES⁺, short acidic): 1.46 min, m/z 306.0/308.0 [M + H]⁺ (100%) |
| Int-32 | 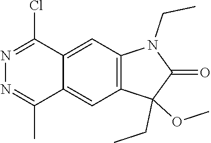 | UPLC-MS (ES⁺, short acidic): 1.56 min, m/z 306.0/308.0 [M + H]⁺ (100%) |

### 8-chloro-1-cyclopropyl-3-methoxy-3,5-dimethyl-pyrrolo[3,2-g]phthalazin-2-one (Int-33)

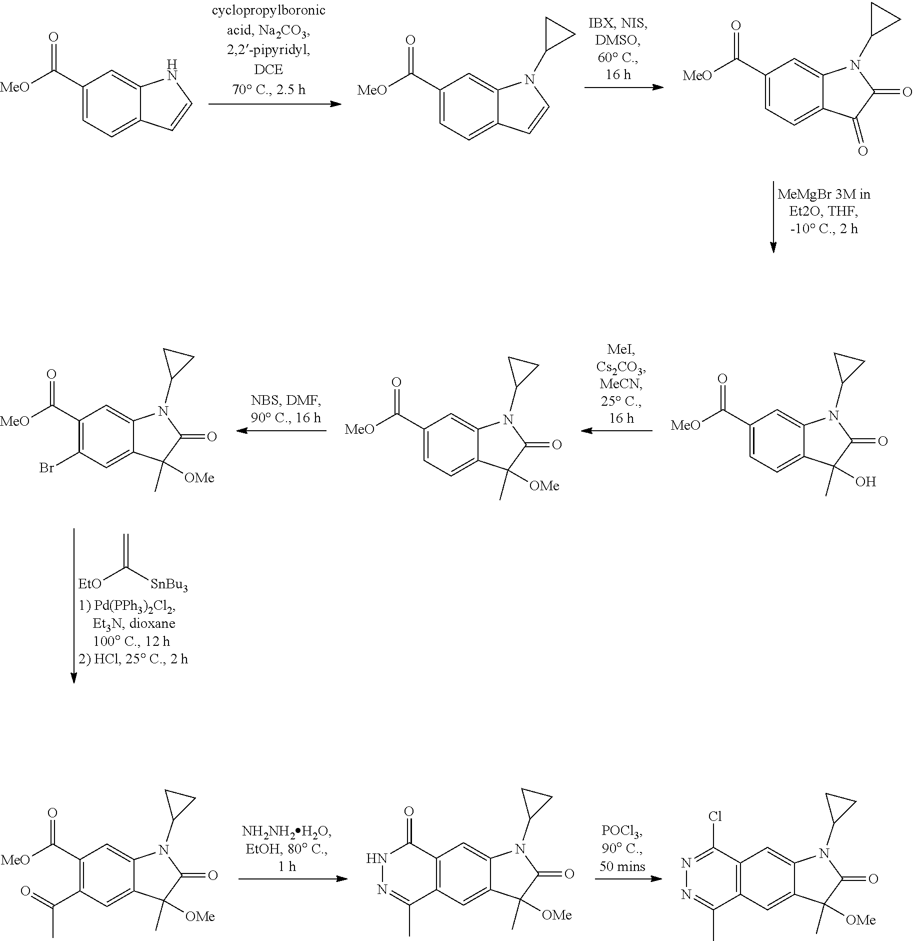

### Step 1

A solution of Methyl 1H-indole-6-carboxylate (500.mg, 2.85 mmol), Copper(II) acetate (544.3 mg, 3.mmol), cyclopropylboronic acid (490 mg, 5.71 mmol), 2,2′-bipyridyl (468 mg, 3.mmol) and sodium carbonate (665 mg, 6.28 mmol) in DCE (6 mL) was bubbled with air and stirred at 70° C. for 2.5 hours and at RT overnight.

The reaction was quenched with sat. aq. NH₄Cl, extracted with DCM (×3). Organic layers were combined, washed with brine, passed through a phase separator and concentrated in vacuo. The crude product was purified by column chromatography (25 g, wet-load, using as eluent a gradient 0-50% EtOAc in petroleum ether) affording methyl 1-cyclopropylindole-6-carboxylate (464 mg, 2.16 mmol, 75.5% yield) as a pale yellow oil.

UPLC-MS (ES⁺, Short acidic): 2.04 min, m/z 216.0 [M+H]⁺ (97%)

1H NMR (400 MHz, CDCl₃): 8.32-8.29 (m, 1H), 7.81 (dd, J 1.5, 8.3 Hz, 1H), 7.60 (dd, J=0.6, 8.3 Hz, 1H), 7.28 (d, J=3.1 Hz, 1H), 6.46 (dd, J=0.8, 3.2 Hz, 1H), 3.96 (s, 3H), 3.40 (septet, J=3.7 Hz, 1H), 1.17-0.99 (m, 4H)

### Step 2

Made in the same way as 8-chloro-3-methoxy-1,3,5-trimethyl-pyrrolo[3,2-g]phthalazin-2-one Step 2. Methyl 1-cyclopropyl-2,3-dioxo-indoline-6-carboxylate (Quantitative yield) as a red solid.

UPLC-MS (ES⁺, Short acidic): 1.64 min, m/z 245.9 [M+H]⁺ (75%)

### Step 3

Made in the same way as 8-chloro-3-methoxy-1,3,5-trimethyl-pyrrolo[3,2-g]phthalazin-2-one Step 3. methyl 1-cyclopropyl-3-hydroxy-3-methyl-2-oxo-indoline-6-carboxylate (1.88 g, 7.20 mmol, 47.7% yield) as a yellow solid.

UPLC-MS (ES⁺, Short acidic): 1.40 min, m/z 262.0 [M+H]⁺ (100%)

1H NMR (400 MHz, DMSO-d₆7.71 (dd, J=1.5, 7.6 Hz, 1H), 7.58 (d, J=1.1 Hz, 1H), 7.47 (d, J=7.6 Hz, 1H), 6.05 (s, 1H), 3.87 (s, 3H), 2.76-2.67 (m, 1H), 1.36 (s, 3H), 1.06-0.98 (m, 2H), 0.84-0.71 (m, 2H)

### Step 4

Made in the same way as 8-chloro-3-methoxy-1,3,5-trimethyl-pyrrolo[3,2-g]phthalazin-2-one Step 4. methyl 1-cyclopropyl-3-methoxy-3-methyl-2-oxo-indoline-6-carboxylate (Quantitative yield) as a yellow oil.

UPLC-MS (ES⁺, Short acidic): 1.70 min, m/z 276.0 [M+H]⁺ (100%)

1H NMR (400 MHz, CDCl₃): 7.86-7.81 (m, 1H), 7.75-7.73 (m, 1H), 7.37 (d, J=7.7 Hz, 1H), 3.95 (s, 3H), 3.00 (s, 3H), 2.69 (septet, J=3.6 Hz, 1H), 1.52 (s, 3H), 1.18-1.09 (m, 2H), 0.97-0.88 (m, 2H)

### Step 5

Made in the same way as 8-chloro-3-methoxy-1,3,5-trimethyl-pyrrolo[3,2-g]phthalazin-2-one Step 5. methyl 5-bromo-1-cyclopropyl-3-methoxy-3-methyl-2-oxo-indoline-6-carboxylate (1.4 g, 2.13 mmol, 25.7% yield) as a yellow oil. The product was carried on to the next step without further purification.

UPLC-MS (ES⁺, Short acidic): 1.83 min, m/z 354/356 [M+H]⁺ (54%)

### Step 6

Made in the same way as 8-chloro-3-methoxy-1,3,5-trimethyl-pyrrolo[3,2-g]phthalazin-2-one Step 6. methyl 5-acetyl-1-cyclopropyl-3-methoxy-3-methyl-2-oxo-indoline-6-carboxylate (Quantitative yield) as a pale yellow solid.

UPLC-MS (ES⁺, Short acidic): 1.61 min, m/z 318.0 [M+H]⁺ (100%)

### Step 7

Made in the same way 8-chloro-3-methoxy-1,3,5-trimethyl-pyrrolo[3,2-g]phthalazin-2-one Step 7. 1-cyclopropyl-3-methoxy-3,5-dimethyl-7H-pyrrolo[3,2-g]phthalazine-2,8-dione (158 mg, 0.53 mmol, 90.1% yield) as a grey solid.

UPLC-MS (ES⁺, Short acidic): 1.41 min, m/z 300.1 [M+H]⁺ (100%)

### Step 8

Made in the same way as 8-chloro-3-methoxy-1,3,5-trimethyl-pyrrolo[3,2-g]phthalazin-2-one Step 8. 8-chloro-1-cyclopropyl-3-methoxy-3,5-dimethyl-pyrrolo[3,2-g]phthalazin-2-one (163 mg, 0.51 mmol, 97.2% yield) as a pale grey solid.

UPLC-MS (ES⁺, short acidic): 1.56 min, m/z 318.0/320.0 [M+H]⁺ (100%)

The following chlorophthalazine were prepared in a similar manner.

TABLE 3F
Intermediates Prepared According to the Disclosed Methods

| Compound | Structure | Analytical data |
|---|---|---|
| Int-34 | 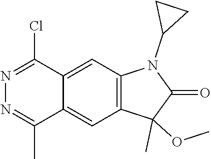 | UPLC-MS (ES⁺, short acidic): 1.59 min, m/z 332/334.1 [M + H]⁺ (100%) |

### 8-chloro-5-ethyl-3-methoxy-1,3-dimethyl-pyrrolo[3,2-g]phthalazin-2-one (Int-35)

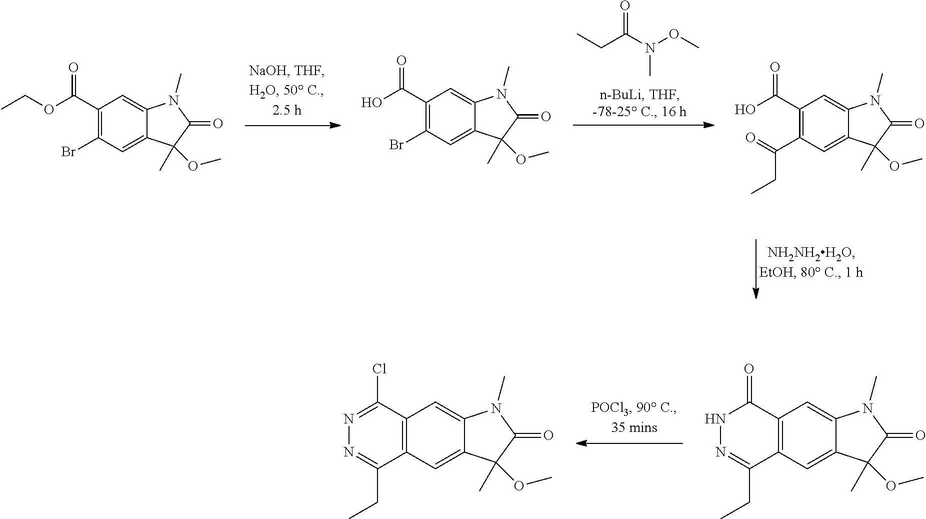

### Step 1

To a stirred solution of ethyl 5-bromo-3-methoxy-1,3-dimethyl-2-oxo-indoline-6-carboxylate (3.78 g, 11.0 mmol) in THF (40 mL) and Water (30 mL) Sodium hydroxide (2.21 g, 55.2 mmol) was added and the reaction mixture was stirred at 50° C. for 2.5 h. The reaction mixture was cooled and the pH was adjusted to <6 via addition of 1M HCl. EtOAc was added and the two phases were separated. The aqueous was then extracted with EtOAc (×2), the combined organic extracts were dried over magnesium sulphate, filtered and concentrated in vacuo to afford 5-bromo-3-methoxy-1,3-dimethyl-2-oxo-indoline-6-carboxylic acid (3.19 g, 10.2 mmol, 91.9% yield) as a yellow solid.

UPLC-MS (ES⁺, Short acidic): 1.39 min, m/z 313.8/311.8 [M−H]⁻ (100%)

### Step 2

To a stirring solution of 5-bromo-3-methoxy-1,3-dimethyl-2-oxo-indoline-6-carboxylic acid (1.00 g, 3.18 mmol) in THF (50 mL) under nitrogen atmosphere at −78° C. was added dropwise n-Butyllithium solution (2.93 mL, 7.32 mmol). The reaction was stirred for 30 min then N-Methoxy-N-methylpropionamide (1.14 mL, 9.55 mmol) was added. The mixture was stirred at −78° C. for 1 h. The reaction mixture was warmed to r.t and stirred at r.t overnight. The reaction mixture was quenched with 1N HCl and extracted with EtOAc, (×3). The combined organic extracts were dried over MgSO₄, filtered and concentrated in vacuo to afford 3-methoxy-1,3-dimethyl-2-oxo-5-propanoyl-indoline-6-carboxylic acid Reaction assumed quantitative and telescoped through to the next step without further purification.

UPLC-MS (ES⁺, Short acidic): 1.26 min, m/z 292.0 [M+H]⁺ (28%)

### Step 3

To a stirring solution of 3-methoxy-1,3-dimethyl-2-oxo-5-propanoyl-indoline-6-carboxylic acid (1.85 g, 6.36 mmol) in Ethanol (50 mL) was added Hydrazine Hydrate (0.77 mL, 15.9 mmol). The reaction mixture was heated to 80° C. for 3 hours.

The reaction was concentrated in vacuo. The crude material was purified via flash chromatography (25 g, 0-100% EtOAc in Pet Ether) like fractions combined to afford 5-ethyl-3-methoxy-1,3-dimethyl-7H-pyrrolo[3,2-g]phthalazine-2,8-dione (170 mg, 0.59 mmol, 9.30% yield) as a white solid

UPLC-MS (ES⁺, Short acidic): 1.53 min, m/z 288.0 [M+H]⁺ (97%)

### Step 4

A stirring suspension of 5-ethyl-3-methoxy-1,3-dimethyl-7H-pyrrolo[3,2-g]phthalazine-2,8-dione (170.mg, 0.59 mmol) in phosphorus oxychloride (0.55 mL, 5.92 mmol) was heated to 90° C. for 35 minutes. POCl₃ was removed in vacuo. The reaction was cooled down with an ice-water bath and quenched with sat. aq. Na₂CO₃. The solid was filtered, washed with water and dried to afford 8-chloro-5-ethyl-3-methoxy-1,3-dimethyl-pyrrolo[3,2-g]phthalazin-2-one (100 mg, 0.33 mmol, 55.3% yield) as a pale grey solid.

UPLC-MS (ES⁺, short acidic): 1.55 min, m/z 306.0/308.0 [M+H]⁺ (100%)

### 5-chloro-1,3,3,8-tetramethyl-pyrrolo[2,3-g]phthalazin-2-one (Int-36)

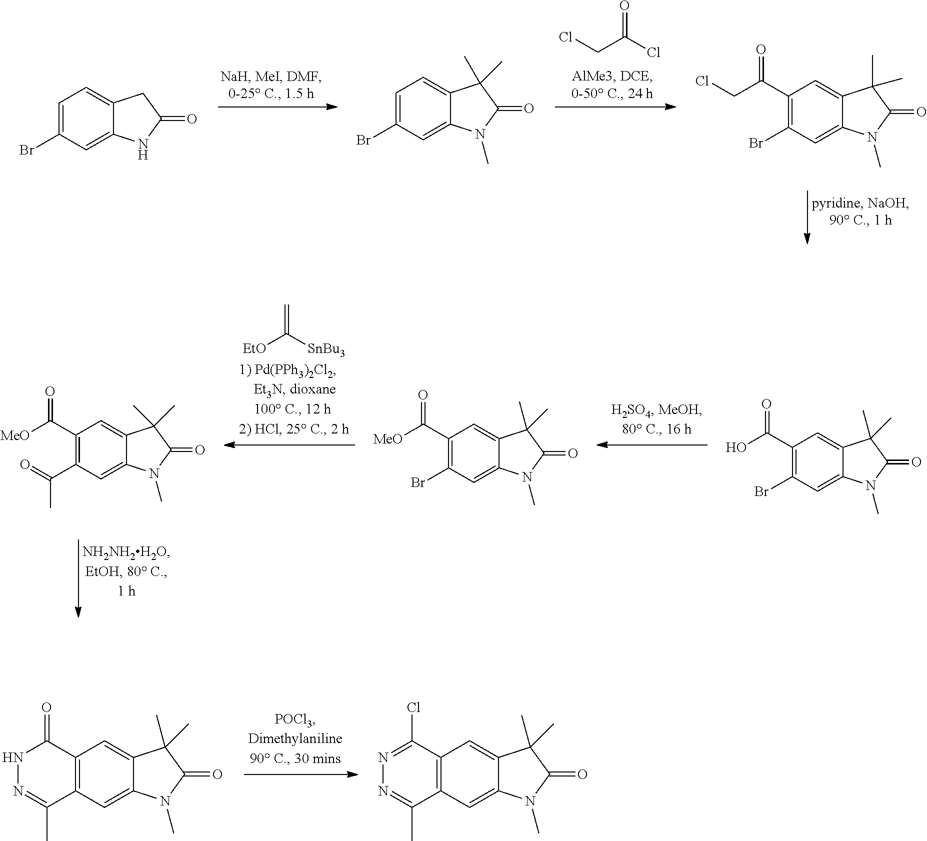

### Step 1

Sodium hydride (755 mg, 18.9 mmol) and dry THF (4 mL) were mixed under inert atmosphere. A suspension of 6-bromoindolin-2-one (1.g, 4.72 mmol) in THF (10 mL) was added dropwise. The mixture was stirred at room temperature for 20 min. The reaction was cooled to 0 deg then Iodomethane (1.17 mL, 18.9 mmol) was added dropwise. The suspension was stirred at rt overnight. The reaction mixture was cooled and quenched with aq. NH4Cl (4 mL) and sat. aq. NaHCO₃ were added followed by extraction with EtOAc (×3). The combined organic layers were washed with brine, dried over Na₂SO₄ and the solvent removed under reduce pressure. The crude was purified by column chromatography using as eluent a gradient 0-30% EtOAc in petroleum ether to give 6-bromo-1,3,3-trimethyl-indolin-2-one (1.05 g, 4.15 mmol, 87.9% yield) as a white solid.

UPLC-MS (ES⁺, short acidic): 1.93 min, m/z 253.9/255.9 [M+H]⁺ (100%).

1H-NMR (400 MHz, 400 MHz, CDCl₃): 7.19 (dd, J=1.7, 7.8 Hz, 1H), 7.06 (d, J=7.8 Hz, 1H), 6.99 (d, J=1.7 Hz, 1H), 3.19 (s, 3H), 1.35 (s, 6H).

### Step 2

To a suspension of 6-bromo-1,3,3-trimethyl-indolin-2-one (1.06 g, 4.15 mmol) and aluminium chloride (1.99 g, 15.0 mmol) in DCE (12.6 mL), chloroacetyl chloride (0.66 mL, 8.30 mmol) was added dropwise at 0° C. The resulting mixture was stirred at 0° C. for 20 min and then at 50° C. overnight. The reaction was cooled to 0° C. aluminium chloride (2.77 g, 20.8 mmol) and chloroacetyl chloride (0.99 mL, 12.5 mmol) were added and the reaction stirred at 0° C. for 20 min and then 50° C. for 4 h. Water/ice were added followed by extraction with DCM (×3). The combined organic layers were washed with brine, dried over Na₂SO₄, filtered and the solvent removed under reduce pressure. The crude was purified by column chromatography (12 g, eluting in 0-100% EtOAc in petroleum ether) to give 6-bromo-5-(2-chloroacetyl)-1,3,3-trimethyl-indolin-2-one (1.06 g, 3.21 mmol, 77.2% yield) as a yellow solid.

UPLC-MS (ES⁺, short acidic): 1.75 min, m/z 329.9/332.0/334.3 [M+H]⁺ (90%).

1H-NMR (400 MHz, DMSO-d6): 7.39 (s, 1H), 7.09 (s, 1H), 4.73 (s, 2H), 3.23 (s, 3H), 1.39 (s, 6H).

### Step 3

A mixture of 6-bromo-5-(2-chloroacetyl)-1,3,3-trimethyl-indolin-2-one (1.06 g, 3.21 mmol) and Pyridine (7.8 mL, 96.2 mmol) was heated to 90° C. for 30 min. The solvent was removed under reduce pressure and stripped with toluene (×2). To the resulting solid, aq. 2.5M Sodium hydroxide (10.9 mL, 27.3 mmol) was added and the resulting mixture heated to 80° C. for 30 min.

The reaction was cooled to RT and acidified using aq. 2M HCl to pH 2-3. The resulting precipitate was collected by vacuum filtration, washed with water. The solid was dissolved in MeOH, dried over Na₂SO₄ and the solvent removed under reduce pressure to give 6-bromo-1,3,3-trimethyl-2-oxo-indoline-5-carboxylic acid (987 mg, 2.98 mmol, 92.9% yield) as a dark brown solid.

UPLC-MS (ES⁺, short acidic): 1.41 min, m/z 298.0/300.0 [M+H]⁺ (87%)

¹H-NMR (400 MHz, DMSO-d₆): 7.72 (s, 1H), 7.33 (s, 1H), 3.15 (s, 3H), 1.28 (s, 6H).

### Step 4

To a suspension of 6-bromo-1,3,3-trimethyl-2-oxo-indoline-5-carboxylic acid (987 mg, 2.98 mmol) in Methanol (13 mL) was added Sulfuric acid (3.18 mL, 59.6 mmol) was added and the reaction stirred at 80° C. for 6 h. The mixture was cooled to RT, MeOH was evaporated, and the residue was neutralised with sat. aq. NaHCO₃ followed by extraction with EtOAc (×3). The organic phases were washed with brine, dried over Na₂SO₄, filtered and the solvent removed under reduce pressure to give methyl 6-bromo-1,3,3-trimethyl-2-oxo-indoline-5-carboxylate (930 mg, 2.98 mmol, 100% yield) as a brown solid.

UPLC-MS (ES⁺, short acidic): 1.73 min, m/z 312.0/314.0 [M+H]⁺ (83%).

1H-NMR (400 MHz, CDCl3): 7.71 (s, 1H), 7.13 (s, 1H), 3.93 (s, 3H), 3.22 (s, 3H), 1.38 (s, 6H).

### Step 5

To a stirring, thoroughly degassed solution of methyl 6-bromo-1,3,3-trimethyl-2-oxo-indoline-5-carboxylate (930 mg, 2.98 mmol), Tributyl(1-ethoxyvinyl)tin (1.21 mL, 3.58 mmol), triethylamine (1.04 mL, 7.45 mmol) in dry 1,4-Dioxane (35 mL) was added Bis(triphenylphosphine)palladium(II) dichloride (209 mg, 0.30 mmol). Reaction heated to 100° C. overnight and then at rt for 2 days. The RM was cooled to RT and aq. 2M Hydrogen Chloride (7.45 mL, 14.9 mmol) was added and stirred for 45 min. Water and EtOAc were added, and the layers were separated. The aqueous was extracted with EtOAc (×3). Combined organic extracts were washed with brine, dried over Na₂SO₄, filtered and concentrated. The crude was purified by column chromatography (eluting in 0-60% EtOAc in petroleum ether) to yield methyl 6-acetyl-1,3,3-trimethyl-2-oxo-indoline-5-carboxylate (524 mg, 1.90 mmol, 63.9% yield) as a pale yellow solid.

UPLC-MS (ES⁺, Short acidic): 1.54 min, m/z 276.1 [M+H]⁺ (97%).

¹H-NMR (400 MHz, DMSO-d₆): δ 7.78 (s, 1H), 6.72 (s, 1H), 3.90 (s, 3H), 3.24 (s, 3H), 2.54 (s, 3H). 1.40 (s, 6H).

### Step 6

To a stirring solution of methyl 6-acetyl-1,3,3-trimethyl-2-oxo-indoline-5-carboxylate (524 mg, 1.90 mmol) in Ethanol (12 mL) was added hydrazine Hydrate (139 μL, 2.86 mmol).

Reaction was stirred at 80° C. for 2 h. All volatiles removed under reduced pressure. Remaining solid was then washed with MTBE, filtered, and dried to give 1,3,3,8-tetramethyl-6H-pyrrolo[2,3-g]phthalazine-2,5-dione (423 mg, 1.64 mmol, 86.4% yield) as an off-white solid.

UPLC-MS (ES⁺, Short acidic): 1.24 min, m/z 258.1 [M+H]⁺ (100%).

¹H-NMR (400 MHz, DMSO-d6): δ 12.33 (s, 1H), 8.21 (s, 1H), 7.36 (s, 1H), 3.29 (s, 3H), 2.54 (s, 3H), 1.36 (s, 6H).

### Step 7

To 1,3,3,8-tetramethyl-6H-pyrrolo[2,3-g]phthalazine-2,5-dione (423 mg, 1.64 mmol) in phosphorus oxychloride (12.3 mL, 131.5 mmol) was added dimethylaniline (0.42 mL, 3.29 mmol) and the reaction heated to 90° C. for 30 min. POCl₃ was removed under reduced pressure.

An ice cold sat. aq. NaHCO₃ was added till pH ˜7-8. The solid was collected by vacuum filtration, dried under vacuum to give 5-chloro-1,3,3,8-tetramethyl-pyrrolo[2,3-g]phthalazin-2-one (552 mg, 1.68 mmol, 100% yield) as a cream solid.

UPLC-MS (ES⁺, Short acidic): 1.46 min, m/z 276.1/278.0 [M+H]⁺ (100%).

1H-NMR (400 MHz, DMSO-d₆): δ 8.26 (s, 1H), 7.66 (s, 1H), 3.34 (s, 3H), 2.92 (s, 3H), 1.43 (s, 6H)

### 5-chloro-1,8-dimethyl-spiro[pyrrolo[2,3-g]phthalazine-3,4′-tetrahydropyran]-2-one (Int-37)

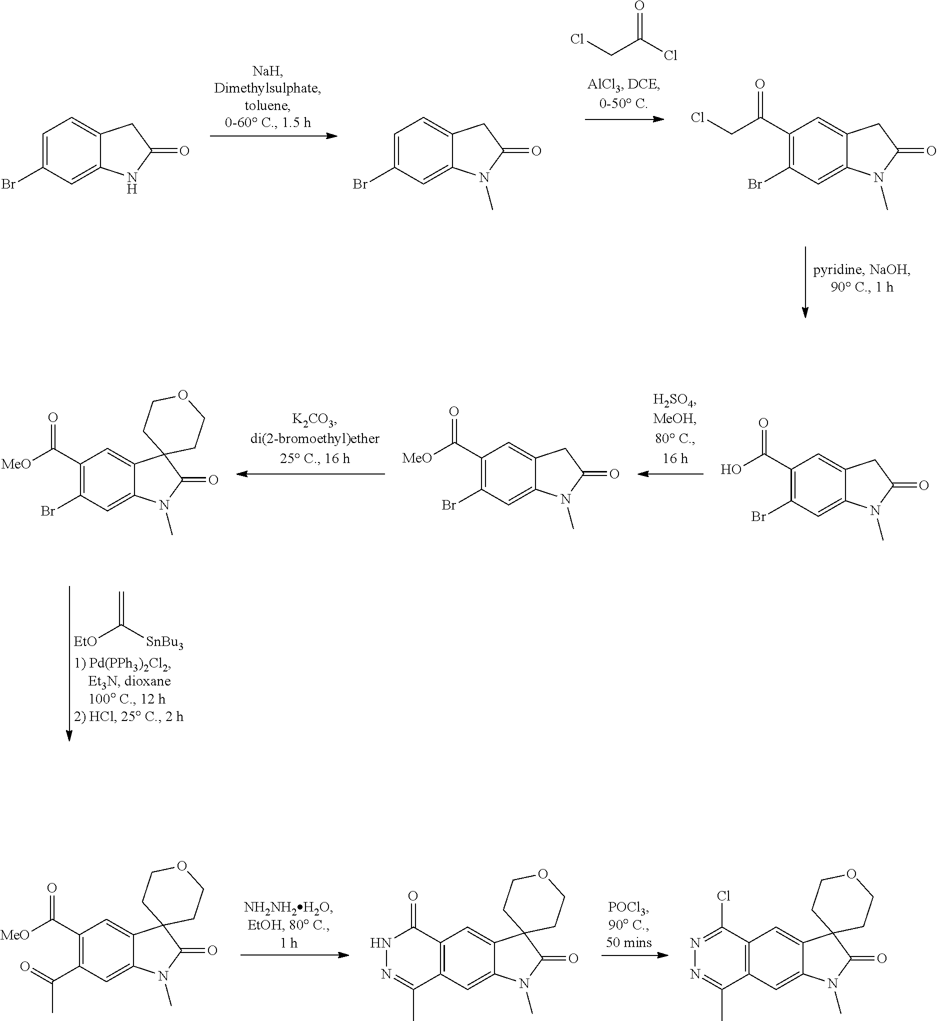

### Step 1

A solution of 6-Bromo-2-oxindole (1.g, 4.72 mmol) in Toluene (31.4 mL) was cooled to 0° C., Sodium hydride, (60% dispersed in mineral oil) (283 mg, 7.07 mmol) was added portionwise. The reaction mixture was stirred for 10 min at 0° C. and then stirred at RT for 30 min. Dimethyl sulphate (0.58 mL, 6.13 mmol) was added and the reaction mixture was heated up to 60° C. for 1 h

The reaction mixture was cooled to 0° C. and quenched with water, then diluted with EtOAc The two phases were separated and the aqueous was extracted with EtOAc (2×). The combined organic extracts were washed with sat aqueous solution of NaHCO₃ and then brine, passed through a hydrophobic filter and concentrated under reduced pressure. The crude product was purified by flash column chromatography (12 g, 0-100% EtOAc in petroleum ether) affording 6-bromo-1-methyl-indolin-2-one (827 mg, 3.66 mmol, 77.5% yield) as light brown solid.

UPLC-MS (ES⁺, Short acidic): 1.60 min, m/z 227.9 [M+H]⁺ (100%)

1H NMR (400 MHz, DMSO-d6) δ 7.23-7.22 (m, 1H), 7.19-7.18 (m, 2H), 3.52 (s, 2H), 3.11 (s, 3H).

### Step 2

Made in the same way as 5-chloro-1,3,3,8-tetramethyl-pyrrolo[2,3-g]phthalazin-2-one Step 2. 6-bromo-5-(2-chloroacetyl)-1-methyl-indolin-2-one (729 mg, 2.42 mmol, 85.9% yield) as a brown solid.

UPLC-MS (ES⁺, Short acidic): 1.66 min, m/z 303.9 [M+H]⁺ (90%)

1H NMR (400 MHz, DMSO-d₆) δ 7.72 (s, 1H), 7.41 (s, 1H), 5.00 (s, 2H), 3.59 (s, 2H), 3.15 (s, 3H).

### Step 3

Made in the same way as 5-chloro-1,3,3,8-tetramethyl-pyrrolo[2,3-g]phthalazin-2-one Step 3. 6-bromo-1-methyl-2-oxo-indoline-5-carboxylic acid (1.78 g, 6.58 mmol, 89.2% yield) as a dark brown solid.

UPLC-MS (ES⁺, Short acidic): 1.28 min, m/z 271.9 [M+H]⁺ (61%)

1H NMR (400 MHz, DMSO-d6) δ 13.04 (s, 1H), 7.70 (s, 1H), 7.34 (s, 1H), 3.57 (s, 2H), 3.14 (s, 3H)

### Step 4

Made in the same way as 5-chloro-1,3,3,8-tetramethyl-pyrrolo[2,3-g]phthalazin-2-one Step 4. methyl 6-bromo-1-methyl-2-oxo-indoline-5-carboxylate (1.12 g, 3.93 mmol, 56.8% yield) as a pink solid.

UPLC-MS (ES⁺, Short acidic): 1.45 min, m/z 286.0 [M+H]⁺ (95%)

1H NMR (400 MHz, DMSO-d₆) δ 7.71 (s, 1H), 7.38 (s, 1H), 3.82 (s, 3H), 3.58 (s, 2H), 3.14 (s, 3H).

### Step 5

To a stirred solution of methyl 6-bromo-1-methyl-2-oxo-indoline-5-carboxylate (648 mg, 2.28 mmol) in DMSO at 0° C. Potassium carbonate (1.26 g, 9.12 mmol) and di(2-bromoethyl)ether (0.4 mL, 3.19 mmol) were added. The reaction was stirred at 0° C. for 10 min and then at RT for 16 h.

The reaction mixture was poured onto water, the two layers were separated, and the aqueous layer was extracted with DCM (×3). The combined organic layers were washed with brine (×2), passed through a hydrophobic filter and concentrated under reduced pressure. The crude was purified by column chromatography (12 g, 0-100% EtOAc in pet ether) to afford methyl 6-bromo-1-methyl-2-oxo-spiro[indoline-3,4′-tetrahydropyran]-5-carboxylate (380 mg, 1.07 mmol, 47.1% yield) as a yellow solid.

UPLC-MS (ES⁺, Short acidic): 1.81 min, m/z 356.0 [M+H]⁺ (87%)

1H NMR (400 MHz, DMSO-d₆) δ 7.92 (s, 1H), 7.44 (s, 1H), 4.07-4.02 (m, 2H), 3.85-3.75 (m, 5H), 3.15 (s, 3H), 1.84-1.71 (m, 4H).

### Step 6

Made in the same way as 5-chloro-1,3,3,8-tetramethyl-pyrrolo[2,3-g]phthalazin-2-one Step 5. methyl 6-acetyl-1-methyl-2-oxo-spiro[indoline-3,4′-tetrahydropyran]-5-carboxylate (285 mg, 0.90 mmol, 83.7% yield) as light brown solid.

UPLC-MS (ES⁺, Short acidic): 1.60 min, m/z 318.2 [M+H]⁺ (93%)

¹H NMR (400 MHz, DMSO-d₆) δ 7.92 (s, 1H), 7.20 (s, 1H), 4.11-4.04 (m, 2H), 3.82-3.77 (m, 5H), 3.18 (s, 3H), 1.89-1.82 (m, 2H), 1.76-1.71 (m, 2H)—CH₃ signal under the DMSO peak

### Step 7

Made in the same way as 5-chloro-1,3,3,8-tetramethyl-pyrrolo[2,3-g]phthalazin-2-one Step 6. 1,8-dimethylspiro[6H-pyrrolo[2,3-g]phthalazine-3,4′-tetrahydropyran]-2,5-dione (243 mg, 0.81 mmol, 90.3% yield) as a grey solid.

UPLC-MS (ES⁺, Short acidic): 1.36 min, m/z 300.1 [M+H]⁺ (96%)

### Step 8

Made in the same way as 5-chloro-1,3,3,8-tetramethyl-pyrrolo[2,3-g]phthalazin-2-one Step 7. 5-chloro-1,8-dimethyl-spiro[pyrrolo[2,3-g]phthalazine-3,4′-tetrahydropyran]-2-one (237 mg, 0.75 mmol, 91.8% yield) as a grey solid,

UPLC-MS (ES⁺, short acidic): 1.49 min, m/z 319.1 [M+H]⁺ (100%).

¹H-NMR (400 MHz, DMSO-d₆) δ 8.29 (s, 1H), 7.66 (s, 1H), 4.17-4.09 (m, 2H), 3.88-3.83 (m, 2H), 2.92 (s, 3H), 2.11-2.02 (m, 2H), 1.79 (d, J=13.3 Hz, 2H)—CH₃ under the DMSO peak

### 5-chloro-3-methoxy-1,3,8-trimethyl-pyrrolo[2,3-g]phthalazin-2-one (Int-38)

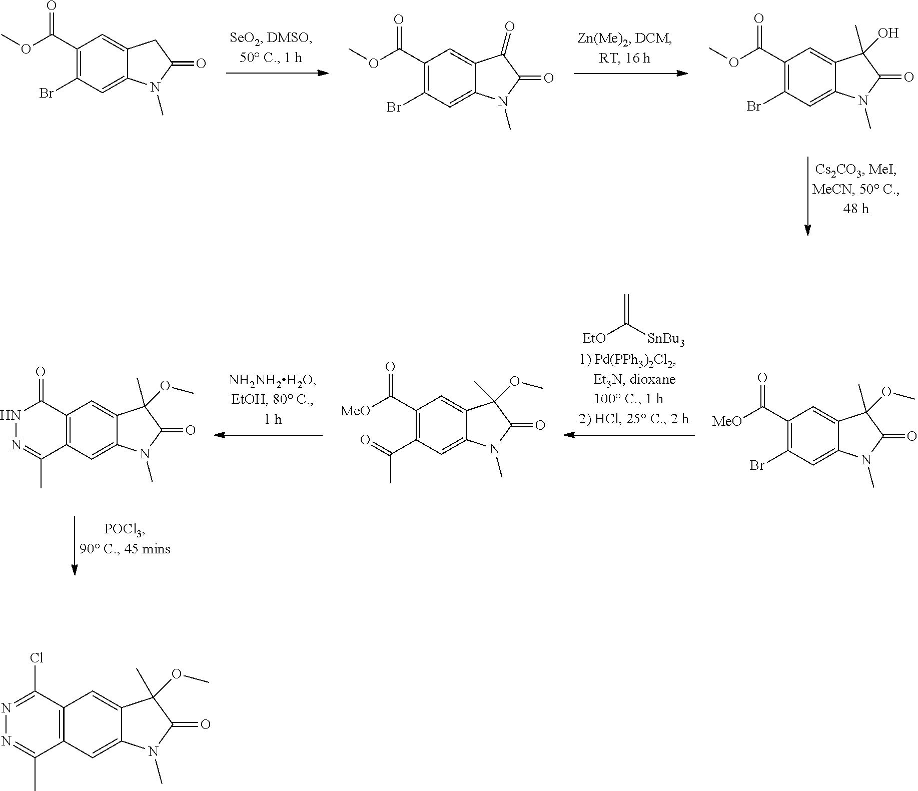

### Step 1

A suspension of methyl 6-bromo-1-methyl-2-oxo-indoline-5-carboxylate (2.73 g, 9.61 mmol) and Selenium dioxide (2.71 g, 24.4 mmol) in DMSO (14 mL) was heated at 50° C. for 1 hour. The reaction mixture was cooled down and water was added. The resulting red precipitate was filtered off, washed with water and dried to afford methyl 6-bromo-1-methyl-2,3-dioxo-indoline-5-carboxylate (quantitative yield) as a red solid.

UPLC-MS (ES⁺, Short acidic, 1.44 min m/z 297.8/299.9 [M+H]⁺ (100%)

1H NMR (400 MHz, DMSO-d₆): 7.87 (s, 1H), 7.63 (s, 1H), 3.84 (s, 3H), 3.17 (s, 3H)

### Step 2

To a stirred solution of methyl 6-bromo-1-methyl-2,3-dioxo-indoline-5-carboxylate (448 mg, 1.5 mmol) in DCM (12 mL) at 0° C. Dimethyl zinc 2M in toluene (5.11 mL, 10.2 mmol) was added dropwise. The reaction mixture was warmed to room temperature and stirred overnight. The reaction mixture was cooled to 0° C. and quenched slowly with saturated aq. of NH₄Cl. The two phases were separated and the aqueous was extracted with EtOAc (×3). The combined organic extracts were washed with brine, dried over Na₂SO₄, filtered and concentrated in vacuo to afford:methyl 6-bromo-3-hydroxy-1,3-dimethyl-2-oxo-indoline-5-carboxylate (443 mg, 1.18 mmol, 78.9% yield) as a brown solid.

UPLC-MS (ES⁺, Short acidic): 1.49 min, m/z 313.9, 315.9 [M+H]⁺ (37%)

1H NMR (400 MHz, DMSO-d₆) 7.77 (s, 1H), 7.43 (s, 1H), 6.13 (s, 1H), 3.83 (s, 3H), 3.14 (s, 3H), 1.40 (s, 3H)

### Step 3

To a suspension of methyl 6-bromo-3-hydroxy-1,3-dimethyl-2-oxo-indoline-5-carboxylate (332.mg, 1.06 mmol) in MeCN (6.7 mL) was added Cesium Carbonate (584 mg, 4.23 mmol) followed by Iodomethane (0.33 mL, 5.28 mmol). The reaction mixture was heated at 50° C. for 16 h. H₂O and DCM were added, the phases were separated. The aqueous phase was extracted with DCM (3×). The combined organic extracts were passed through a phase separator and concentrated in vacuo. The crude material was purified by column chromatography (eluting in 0-100% EtOAc in petroleum ether) to afford methyl 6-bromo-3-methoxy-1,3-dimethyl-2-oxo-indoline-5-carboxylate (300 mg, 0.91 mmol, 86.5% yield) as an off-white solid.

UPLC-MS (ES⁺, Short acidic): 1.69 min, (100%)

1H NMR (400 MHz, DMSO-d₆) 7.77 (s, 1H), 7.52 (s, 1H), 3.84 (s, 3H), 3.18 (s, 3H), 2.87 (s, 3H), 1.43 (s, 3H)

### Step 4

A solution of methyl 6-bromo-3-methoxy-1,3-dimethyl-2-oxo-indoline-5-carboxylate (168.mg, 0.51 mmol), Tributyl(1-ethoxyvinyl)tin (0.21 mL, 0.61 mmol) and Triethylamine (0.21 mL, 1.54 mmol) in 1,4-Dioxane (5 mL) was degassed with N₂ for 10 minutes. Bis(triphenylphosphine)palladium(II) dichloride (36 mg, 0.05 mmol) was added, the reaction mixture was degassed with N₂ then was heated at 100° C. for 1 hour. The reaction mixture was cooled down and Hydrogen Chloride (0.21 mL, 2.56 mmol) was added. The reaction mixture was stirred at room temperature overnight. Water was added and the aqueous phase was extracted with DCM (3×). The combined organic extracts were washed with brine, passed through a phase separator and evaporated to dryness. The residue was purified by flash chromatography (4 g cartridge, eluent ethyl acetate in petroleum ether 0-100%) to afford methyl 6-acetyl-3-methoxy-1,3-dimethyl-2-oxo-indoline-5-carboxylate (122 mg, 0.42 mmol, 81.8% yield) as a pale pink solid.

UPLC-MS (ES⁺, Short acidic): 1.37 min, m/z no mass [M+H]⁺ (85%)

1H NMR (400 MHz, DMSO-d₆) 7.80 (s, 1H), 7.25 (s, 1H), 3.82 (s, 3H), 3.20 (s, 3H), 2.89 (s, 3H), 1.46 (s, 3H), CH₃ under solvent peak

### Step 5

To a solution of methyl 6-acetyl-3-methoxy-1,3-dimethyl-2-oxo-indoline-5-carboxylate (122.mg, 0.42 mmol) in Ethanol (3.5 mL) was added Hydrazine Hydrate (0.04 mL, 0.5 mmol). The reaction mixture was heated at 80° C. for 2 hours. The reaction mixture was evaporated to dryness and the residue was dissolved in a minimum amount of ethanol then cooled in an ice bath. The grey solid was collected and dried to afford 3-methoxy-1,3,8-trimethyl-6H-pyrrolo[2,3-g]phthalazine-2,5-dione (75 mg, 0.27 mmol, 65.5% yield).

UPLC-MS (ES⁺, Short acidic): 1.14 min, m/z 274.1 [M+H]⁺ (100%) Step 6

A suspension of 3-methoxy-1,3,8-trimethyl-6H-pyrrolo[2,3-g]phthalazine-2,5-dione (75.mg, 0.27 mmol) in phosphorus oxychloride (0.8 mL, 8.58 mmol) was heated at 90° C. for 1 hour.

The reaction mixture was cooled down and quenched with ice then with sat. aq. NaHCO₃. The aqueous phase was extracted with dichloromethane (3×), the organic phases were washed with brine and evaporated to dryness to afford 5-chloro-3-methoxy-1,3,8-trimethyl-pyrrolo[2,3-g]phthalazin-2-one (80 mg, 0.26 mmol, 92.9% yield) as a grey solid.

UPLC-MS (ES⁺, Short acidic): 1.29 min, m/z 293.0/294.0 [M+H]⁺ (93%)

The following chlorophthalazine were prepared in a similar manner.

TABLE 3G
Intermediates Prepared According to the Disclosed Methods

| Compound | Structure | Analytical data |
|---|---|---|
| Int-39 | 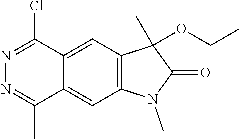 [SMILES: CCOC1(C)C(=O)N(C)c2cc3c(C)nnc(Cl)c3cc21] | UPLC-MS (ES⁺, short acidic,): 1.65 min, m/z 306.0/308.0 [M + H]+ (100%) |

### 5-chloro-3-methoxy-3-ethyl-1,8-dimethyl-pyrrolo[2,3-g]phthalazin-2-one (Int-40)

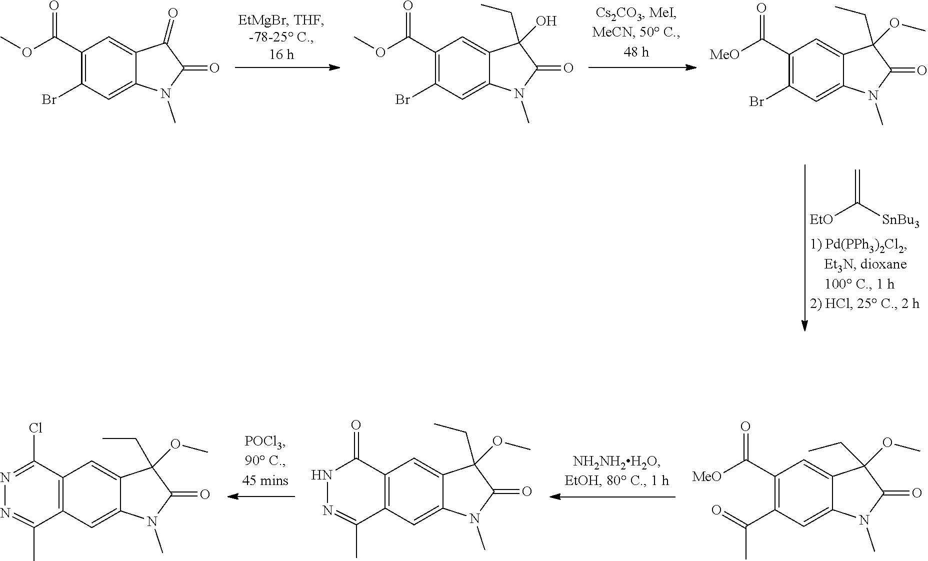

### Step 1

A solution of methyl 6-bromo-1-methyl-2,3-dioxo-indoline-5-carboxylate (500.mg, 1.68 mmol) in THF (8 mL) was cooled to −78° C., a 1M solution in THF of Ethylmagnesium bromide (1.68 mL, 1.68 mmol) was added dropwise. The reaction mixture was then allowed to warm to 25° C. overnight. The reaction was quenched with sat aq solution of NH₄Cl and diluted with DCM. The two phases were separated, the aqueous layer extracted with DCM (×3), organic extracts were combined, washed with brine, passed through a hydrophobic filter and concentrated under reduced pressure. The crude material was purified by flash column chromatography (25 g, dry loading, 0-100% EtOAc in petroleum ether) to afford methyl 6-bromo-3-ethyl-3-hydroxy-1-methyl-2-oxo-indoline-5-carboxylate (210 mg, 0.64 mmol, 38.2% yield)

UPLC-MS (ES⁺, Short acidic): 1.57 min, m/z 311.9 [M+H−H2O]⁺ (83%)

### Step 2

Made in the same way as 5-chloro-3-methoxy-1,3,8-trimethyl-pyrrolo[2,3-g]phthalazin-2-one Step 3. methyl 6-bromo-3-ethyl-3-methoxy-1-methyl-2-oxo-indoline-5-carboxylate (855 mg, 2.50 mmol, 86.3% yield) as an grey solid,

UPLC-MS (ES⁺, short acidic): 1.91 min, (91%) mass not observed

### Step 3

Made in the same way as 5-chloro-3-methoxy-1,3,8-trimethyl-pyrrolo[2,3-g]phthalazin-2-one Step 4. methyl 6-acetyl-3-ethyl-3-methoxy-1-methyl-2-oxo-indoline-5-carboxylate (622 mg, 2.04 mmol, 81.5% yield) as a white solid

UPLC-MS (ES⁺, short acidic): 1.48 min, m/z 306.1[M+H]⁺ (43%)

### Step 4

Made in the same way as 5-chloro-3-methoxy-1,3,8-trimethyl-pyrrolo[2,3-g]phthalazin-2-one Step 5. 3-ethyl-3-methoxy-1,8-dimethyl-6H-pyrrolo[2,3-g]phthalazine-2,5-dione (515 mg, 1.79 mmol, 88.0% yield) as a grey solid.

UPLC-MS (ES⁺, short acidic): 1.44 min, m/z 288.0 [M+H]⁺ (100%)

### Step 5

Made in the same way as 5-chloro-3-methoxy-1,3,8-trimethyl-pyrrolo[2,3-g]phthalazin-2-one Step 6. 5-chloro-3-ethyl-3-methoxy-1,8-dimethyl-pyrrolo[2,3-g]phthalazin-2-one (443 mg, 1.45 mmol, 99.6% yield) as a grey/brown solid.

UPLC-MS (ES⁺, short acidic): 1.63 min, m/z 306.0 [M+H]⁺ (100%)

### Example 2. Synthesis of Compounds of the Disclosure

### 3-cyclopentyl-1,8-dimethyl-5-[1-[3-(trifluoromethyl)phenyl]-ethylamino]⁻ imidazo[4,5-g]phthalazin-2-one (1)

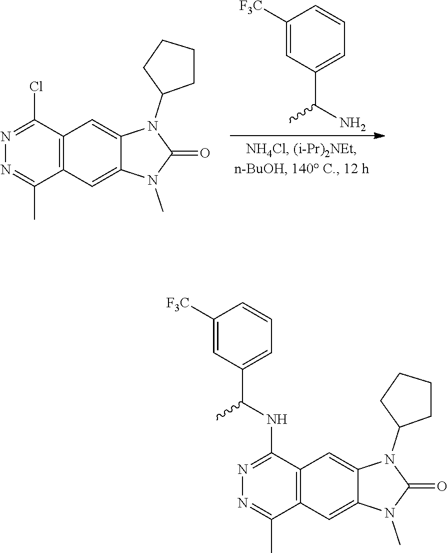

### Step 1

To a stirring solution of 5-chloro-3-cyclopentyl-1,8-dimethyl-imidazo[4,5-g]phthalazin-2-one (90.mg, 0.2800 mmol) in n-butanol (2.2 mL) were added alpha-methyl-3-(trifluoromethyl)benzylamine (54 uL, 0.34 mmol), ammonium chloride (23 mg, 0.4300 mmol) and N,N-diisopropylethylamine (74 uL, 0.43 mmol). The reaction vial was sealed and heated to 140° C. overnight. More alpha-Methyl-3-(trifluoromethyl)benzylamine (54 uL, 0.34 mmol), ammonium chloride (23 mg, 0.43 mmol) and N,N-diisopropylethylamine (74 uL, 0.43 mmol) were added, the tube was sealed, and heated to 140° C. for 1.5 hours under microwave irradiation. All volatiles were removed under reduced pressure and the crude residue was purified by flash chromatography (eluent ethyl acetate in petroleum ether 50-100% then methanol in dichloromethane to 0-20%) followed by a second column (4 g column, eluent methanol in dichloromethane 0-20%) yielded 3-cyclopentyl-1,8-dimethyl-5-[1[-3-(trifluoromethyl)phenyl]ethylamino]imidazo[4,5-g]phthalazin-2-one (8.4 mg, 0.0179 mmol, 6.3% yield) as a yellow solid.

UPLC-MS (ES⁺, Long acidic): 3.47 min, m/z 470.8 [M+H]0 (100%)

¹H NMR (400 MHz, DMSO-d₆): δ 8.14 (s, 1H), 7.78-7.43 (s, 2H), 7.60 (s, 1H), 7.55-7.52 (m, 2H), 7.31 (d, J=7.7 Hz, 1H), 5.59-5.53 (m, 1H), 4.85-4.77 (m, 1H), 3.50 (s, 3H), 2.66 (s, 3H), 2.29-2.21 (m, 2H), 2.10-1.95 (m, 4H), 1.73-1.67 (m, 2H), 1.64 (d, J=7.1 Hz, 3H)

The following examples were prepared in a similar manner, starting from the corresponding chlorophthalazine and respective amine.

TABLE 4A
Compounds Prepared According to the Disclosed Methods

| Compound | Structure | Analytical data |
|---|---|---|
| 2 | 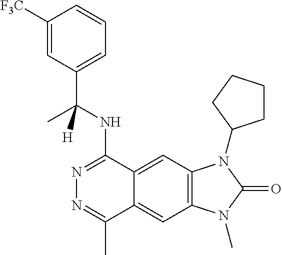 [SMILES: Cc1nnc(N[C@H](C)c2cccc(C(F)(F)F)c2)c2cc3c(cc12)n(C)c(=O)n3C1CCCC1] | UPLC-MS (ES⁺, Long acidic): 3.50 min, m/z 470.7 [M + H]⁺ (97%) ¹H NMR (400 MHz, DMSO-d₆): δ 8.14 (s, 1H), 7.78 (s, 1H), 7.77-7.73 (m, 1H), 7.60 (s, 1H), 7.57-7.52 (m, 2H), 7.32- 7.30 (m, 1H), 5.60-5.52 (m, 1H), 4.85- 4.77 (m, 1H), 3.46 (s, 3H), 2.66 (s, 3H), 1.73-1.69 (m, 2H), 1.64 (d, J = 7.1 Hz, 3H), 1.36-1.28 (m, 2H), 1.24 (s, 2H), 0.88-0.86 (m, 2H) |
| 3 | 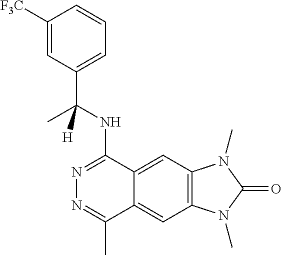 [SMILES: Cc1nnc(N[C@H](C)c2cccc(C(F)(F)F)c2)c2cc3c(cc12)n(C)c(=O)n3C] | UPLC-MS (ES⁺, Long acidic): 3.08 min, m/z 416.6 [M + H]⁺ (100%) ¹H NMR (400 MHz, DMSO-d₆): δ 8.16 (s, 1H), 7.84-7.74 (m, 2H), 7.61 (s, 1H), 7.56-7.52 (m, 2H), 7.36 (d, J = 7.6 Hz, 1H), 5.6-5.5 (m, 1H), 3.51 (s, 3H), 3.48 (s, 3H), 2.66 (s, 3H), 1.63 (d, J = 7.0 Hz, 3H) |
| 4 | 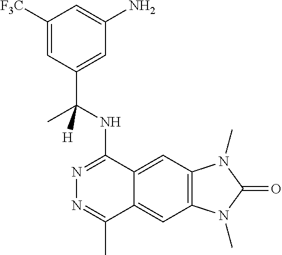 [SMILES: Cc1nnc(N[C@H](C)c2cc(N)cc(C(F)(F)F)c2)c2cc3c(cc12)n(C)c(=O)n3C] | UPLC-MS (ES⁺, Long acidic): 3.06 min, m/z 431.4 [M + H]⁺ (96%) ¹H NMR (400 MHz, DMSO-d₆): δ 8.17 (s, 1H), 7.62 (s, 1H), 7.28-7.20 (m, 1H), 6.92-6.89 (m, 2H), 6.70 (s, 1H), 5.47- 5.39 (m, 1H), 3.51 (s, 3H), 3.49 (s, 3H). 2.71 (s, 3H), 1.60 (d, J = 7.1 Hz, 3H) |
| 5 | 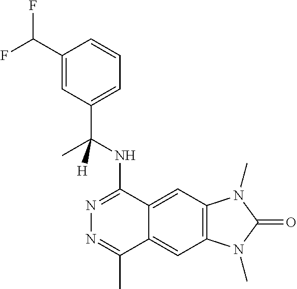 [SMILES: Cc1nnc(N[C@H](C)c2cccc(C(F)F)c2)c2cc3c(cc12)n(C)c(=O)n3C] | UPLC-MS (ES⁺, Long acidic): 3.33 min, m/z 398.3 [M + H]⁺ (95%) ¹H NMR (400 MHz, DMSO-d₆): 8.24 (s, 1H), 7.75-7.67 (m, 1H), 7.66-7.59 (m, 2H), 7.47-7.44 (m, 1H), 7.42-7.39 (m, 1H), 7.01 (t, J = 55.9 Hz, 1H), 5.53- 5.49 (m, 1H), 3.52 (s, 3H), 3.49 (s, 3H), 2.70 (s, 3H), 1.63 (d, J = 7.0 Hz, 3H). |
| 6 | 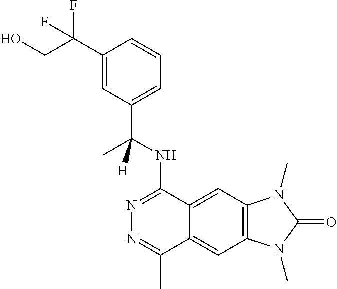 [SMILES: Cc1nnc(N[C@H](C)c2cccc(C(F)(F)CO)c2)c2cc3c(cc12)n(C)c(=O)n3C] | UPLC-MS (ES⁺, Long acidic): 2.50 min, m/z 428.4 [M + H]⁺ (100%) ¹H NMR (400 MHz, DMSO-d₆): 8.17 (s, 1H), 7.62-7.59 (m, 2H), 7.58-7.56 (m, 1H), 7.43-7.38 (m, 1H), 7.36-7.32 (m, 1H), 7.31-7.28 (m, 1H), 5.65-5.52 (m, 2H), 3.88-3.76 (m, 2H), 3.51 (s, 3H), 3.48 (s, 3H), 2.67 (s, 3H), 1.61 (d, J = 7.0 Hz, 3H) |
| 7 | 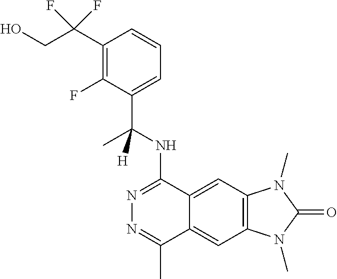 [SMILES: Cc1nnc(N[C@H](C)c2cccc(C(F)(F)CO)c2F)c2cc3c(cc12)n(C)c(=O)n3C] | UPLC-MS (ES⁺, Long acidic): 2.56 min, m/z 446.4 [M + H]⁺ (100%) ¹H NMR (400 MHz, DMSO-d₆): 8.21 (s, 1H), 7.62 (s, 1H), 7.60-7.54 (m, 1H), 7.41-7.32 (m, 2H), 7.21-7.17 (m, 1H), 5.81-5.70 (m, 2H), 4.02-3.90 (m, 2H), 3.52 (s, 3H), 3.49 (s, 3H), 2.66 (s, 3H), 1.62 (d, J = 7.0 Hz, 3H) |
| 8 | 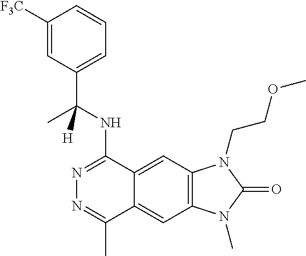 [SMILES: COCCn1c(=O)n(C)c2cc3c(C)nnc(N[C@H](C)c4cccc(C(F)(F)F)c4)c3cc21] | UPLC-MS (ES⁺, Long acidic): 3.08 min, m/z 460.4 [M + H]⁺ (100%) ¹H NMR (400 MHz, DMSO-d₆) 8.21 (s, 1H), 7.80-7.72 (m, 2H), 7.63 (s, 1H), 7.57-7.52 (m, 2H), 7.41-7.35 (m, 1H), 5.63-5.54 (m, 1H), 4.18 (t, J = 5.6 Hz, 2H), 3.76 (t, J = 5.6 Hz, 2H), 3.49 (s, 3H), 3.27 (s, 3H), 2.67 (s, 3H), 1.64 (d, J = 7.0 Hz, 3H) |
| 9 | 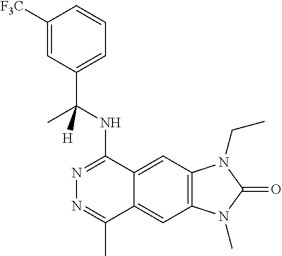 [SMILES: CCn1c(=O)n(C)c2cc3c(C)nnc(N[C@H](C)c4cccc(C(F)(F)F)c4)c3cc21] | UPLC-MS (ES⁺, Long acidic): 3.09 min, m/z 430.4 [M + H]⁺ (98%) ¹H NMR (400 MHz, DMSO-d₆): 8.19 (s, 1H), 7.78 (s, 1H), 7.76-7.73 (m, 1H), 7.62 (s, 1H), 7.55-7.53 (m, 2H), 7.36 (d, J = 7.2 Hz, 1H), 5.61-5.42 (m, 1H), 4.03 (q, J = 7.2 Hz, 2H), 3.48 (s, 3H), 2,66 (s, 3H), 1.64 (d, J - 7.0 Hz, 3H), 1.36 (t, J = 7.1 Hz, 3H) |
| 10 | 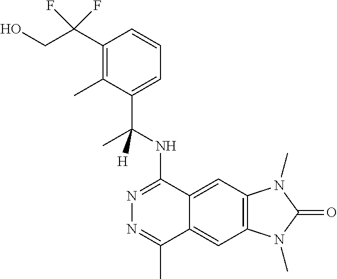 [SMILES: Cc1c([C@@H](C)Nc2nnc(C)c3cc4c(cc23)n(C)c(=O)n4C)cccc1C(F)(F)CO] | UPLC-MS (ES⁺, Long acidic): 2.68 min, m/z 442.6 [M + H]⁺ (100%) ¹H NMR (400 MHz, DMSO-d₆) 8.20 (s, 1H), 7.64 (d, J = 7.5 Hz, 1H), 7.59 (s, 1H), 7.34-7.30 (m, 2H), 7.21 (t, J = 7.7 Hz, 1H), 5.79-5.72 (m, 1H), 5.68 (t, J = 6.4 Hz, 1H), 3.91 (td, J = 14.7, 6.3 Hz, 2H), 3.51 (s, 3H), 3.48 (s, 3H), 2.65 (s, 3H), 2.56 (s, 3H), 1.55 (d, J = 6.9 Hz, 3H). |
| 11 | 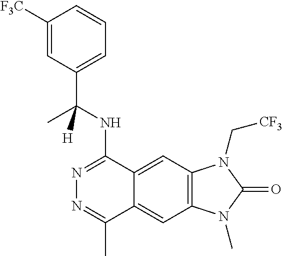 [SMILES: Cc1nnc(N[C@H](C)c2cccc(C(F)(F)F)c2)c2cc3c(cc12)n(C)c(=O)n3CC(F)(F)F] | UPLC-MS (ES⁺, Long acidic): 3.35 min, m/z 484.5 [M + H]⁺ (100%) ¹H NMR (400 MHz, DMSO-d₆) d 8.30 (s, 1H), 7.77 (s, 1H), 7.76-7.72 (m, 1H), 7.69 (s, 1H), 7.57-7.52 (m, 2H), 7.28 (d, J = 7.3 Hz, 1H), 5.64-5.55 (m, 1H), 4.88-4.76 (m, 2H), 3.52 (s, 3H), 2.68 (s, 3H), 1.65 (d, J = 7.0 Hz, 3H) |
| 12 | 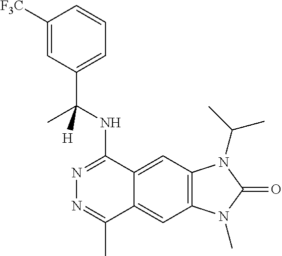 [SMILES: Cc1nnc(N[C@H](C)c2cccc(C(F)(F)F)c2)c2cc3c(cc12)n(C)c(=O)n3C(C)C] | UPLC-MS (ES⁺, Long acidic): 3.33 min, m/z 444.5 [M + H]⁺ (100%) ¹H NMR (400 MHz, DMSO-d₆) d 8.17 (s, 1H), 7.78 (s, 1H), 7.76-7.73 (m, 1H), 7.60 (s, 1H), 7.56-7.53 (m, 2H), 7.42 (d, J = 7.2 Hz, 1H), 5.63-5.56 (m, 1H), 4.79-4.71 (m, 1H), 3.46 (s, 3H), 2.66 (s, 3H), 1.65 (d, J = 7.1 Hz, 3H), 1.63-1.59 (m, 6H) |
| 13 | 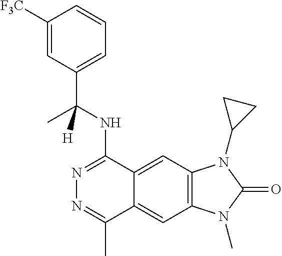 [SMILES: Cc1nnc(N[C@H](C)c2cccc(C(F)(F)F)c2)c2cc3c(cc12)n(C)c(=O)n3C1CC1] | UPLC-MS (ES⁺, Long acidic): 3.18 min, m/z 442.5 [M + H]⁺ (97%) ¹H NMR (400 MHz, DMSO-d₆) d 8.15 (s, 1H), 7.79 (s, 1H), 7.78-7.74 (m, 1H), 7.58 (s, 1H), 7.56-7.51 (m, 3H), 5.62-5.55 (m, 1H), 3.44 (s, 3H), 3.10- 3.04 (m, 1H), 2.66 (s, 3H), 1.65 (d, J = 7.1 Hz, 3H), 1.20-1.16 (m, 2H), 1.08-0.98 (m, 2H) |
| 14 | 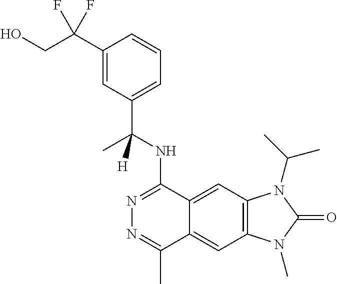 [SMILES: Cc1nnc(N[C@H](C)c2cccc(C(F)(F)CO)c2)c2cc3c(cc12)n(C)c(=O)n3C(C)C] | UPLC-MS (ES⁺, Long acidic): 2.93 min, m/z 456.6 [M + H]⁺ (100%) ¹H NMR (400 MHz, DMSO-d₆) d 8.18 (s, 1H), 7.62-7.55 (m, 3H), 7.44-7.33 (m, 3H), 5.63-5.58 (m, 2H), 4.79- 4.71 (m, 1H), 3.88-3.76 (m, 2H), 3.46 (s, 3H), 2.66 (s, 3H), 1.65-1.58 (m, 9H) |
| 15 | 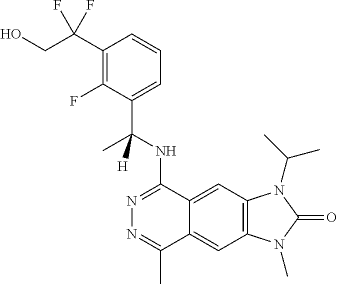 [SMILES: Cc1nnc(N[C@H](C)c2cccc(C(F)(F)CO)c2F)c2cc3c(cc12)n(C)c(=O)n3C(C)C] | UPLC-MS (ES⁺, Long acidic): 2.93 min m/z 474.6 [M + H]⁺ (96%) ¹H NMR (400 MHz, DMSO-d₆) d 8.22 (s, 1H), 7.61 (s, 1H), 7.58-7.55 (m, 1H), 7.43-7.37 (m, 2H), 7.20 (t, J = 7.8 Hz, 1H), 5.79-5.73 (m, 2H), 4.80- 4.72 (m, 1H), 4.03-3.90 (m, 2H), 3.46 (s, 3H), 2.66 (s, 3H), 1.65-1.60 (m, 9H) |
| 16 | 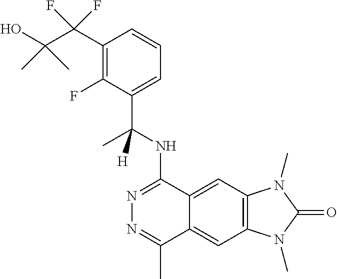 [SMILES: Cc1nnc(N[C@H](C)c2cccc(C(F)(F)C(C)(C)O)c2F)c2cc3c(cc12)n(C)c(=O)n3C] | UPLC-MS (ES⁺, Long acidic): 2.87 min, m/z 474.5 [M + H]⁺ (100%) ¹H NMR (400 MHz, DMSO-d₆): δ 8.24 (s, 1H), 7.66 (s, 1H), 7.58-7.52 (m, 1H), 7.40 (br s, 1H), 7.32-7.26 (m, 1H), 7.19-7.13 (m, 1H), 5.78-5.69 (m, 1H), 5.35 (s, 1H), 3.53 (s, 3H), 3.49 (s, 3H), 2.68 (s, 3H), 1.61 (d, J = 6.9 Hz, 3H), 1.25-1.23 (m, 6H) |
| 17 | 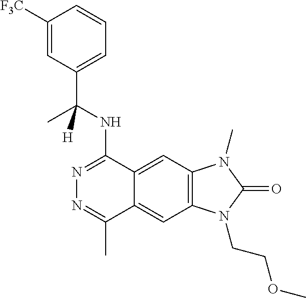 [SMILES: COCCn1c(=O)n(C)c2cc3c(N[C@H](C)c4cccc(C(F)(F)F)c4)nnc(C)c3cc21] | UPLC-MS (ES⁺, Long acidic): 3.13 min, m/z 460.5 [M + H]⁺ (100%) ¹H NMR (400 MHz, DMSO-d₆): δ 8.20 (s, 1H), 7.79 (s, 1H), 7.76-7.73 (m, 1H), 7.70 (s, 1H), 7.57-7.52 (m, 2H), 7.46 (br s, 1H), 5.58-5.51 (m, 1H), 4.18 (t, J = 5.3 Hz, 2H), 3.69 (t, J = 5.3 Hz, 2H), 3.52 (s, 3H), 3.25 (s, 3H), 2.66 (s, 3H), 1.63 (d, J = 7.1 Hz, 3H) |
| 18 | 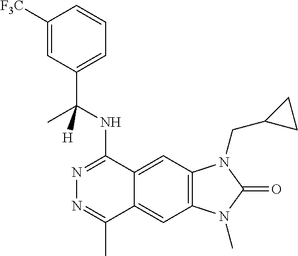 [SMILES: Cc1nnc(N[C@H](C)c2cccc(C(F)(F)F)c2)c2cc3c(cc12)n(C)c(=O)n3CC1CC1] | UPLC-MS (ES+, Long acidic): 3.35 min, m/z 456.5 [M + H]⁺ (100%) ¹H NMR (400 MHz, DMSO-d₆): δ 8.28 (s, 1H), 7.79 (s, 1H), 7.77-7.72 (m, 1H), 7.68 (br s, 1H), 7.59-7.38 (m, 3H), 5.62-5.53 (m, 1H), 3.88 (d, J = 7.1 Hz, 2H), 3.50 (s, 3H), 2.69 (s, 3H), 1.65 (d, J = 7.0 Hz, 3H), 1.44-1.35 (m, 1H), 0.60-0.43 (m, 4H) |
| 19 | 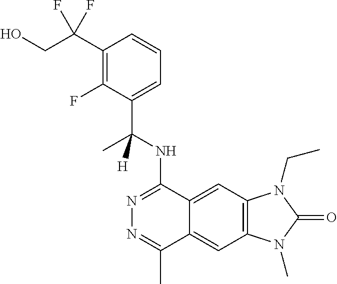 [SMILES: CCn1c(=O)n(C)c2cc3c(C)nnc(N[C@H](C)c4cccc(C(F)(F)CO)c4F)c3cc21] | UPLC-MS (ES⁺, Long acidic): 2.82 min, m/z 460.5 [M + H]⁺ (100%) ¹H NMR (400 MHz, DMSO-d₆): δ 8.24 (s, 1H), 7.64 (s, 1H), 7.60-7.54 (m, 1H), 7.42-7.33 (m, 2H), 7.21-7.18 (m, 1H), 5.79-5.71 (m, 2H), 4.05 (q, J = 7.1 Hz, 2H), 4.01-3.91 (m, 2H), 3.49 (s, 3H), 2.67 (s, 3H), 1.63 (d, J = 7.0 Hz, 3H), 1.38 (t, J = 7.2 Hz, 3H) |
| 20 | 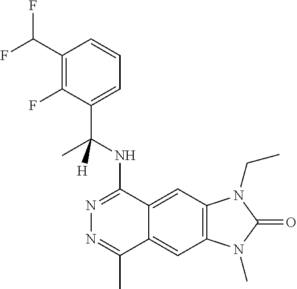 [SMILES: CCn1c(=O)n(C)c2cc3c(C)nnc(N[C@H](C)c4cccc(C(F)F)c4F)c3cc21] | UPLC-MS (ES⁺, Long acidic): 3.12 min, m/z 430.4 [M + H]⁺ (100%) ¹H NMR (400 MHz, DMSO-d₆): δ 8.23 (s, 1H), 7.65-7.57 (m, 2H), 7.49-7.43 (m, 1H), 7.40-7.11 (m, 3H), 5.79-5.69 (m, 1H), 4.05 (q, J = 7.4 Hz, 2H), 3.49 (s, 3H), 2.66 (s, 3H), 1.64 (d, J = 6.7 Hz, 3H), 1.37 (t, J = 7.1 Hz, 3H) |
| 21 | 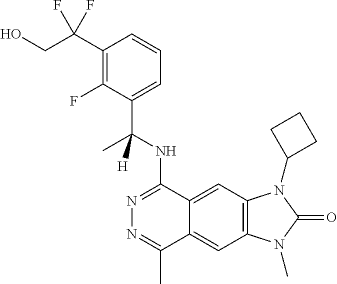 [SMILES: Cc1nnc(N[C@H](C)c2cccc(C(F)(F)CO)c2F)c2cc3c(cc12)n(C)c(=O)n3C1CCC1] | UPLC-MS (ES⁺, Long acidic): 3.06 min, m/z 486.6 [M + H]⁺ (100%) ¹H NMR (400 MHz, DMSO-d₆) 8.22 (s, 1H), 7.61 (s, 1H), 7.59-7.55 (m, 1H), 7.41-7.33 (m, 2H), 7.22-7.17 (m, 1H), 5.78-5.72 (m, 2H), 4.98- 4.89 (m, 1H), 3.96 (td, J = 14.6, 6.5 Hz, 2H), 3.46 (s, 3H), 3.09-2.99 (m, 2H), 2.66 (s, 3H), 2.44-2.36 (m, 2H), 1.98- 1.81 (m, 2H), 1.63 (d, J = 7.0 Hz, 3H). |
| 22 | 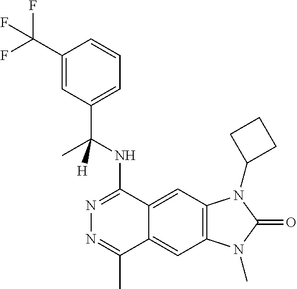 [SMILES: Cc1nnc(N[C@H](C)c2cccc(C(F)(F)F)c2)c2cc3c(cc12)n(C)c(=O)n3C1CCC1] | UPLC-MS (ES⁺, Long acidic): 3.43 min, m/z 456.4 [M + H]⁺ (100%) ¹H NMR (400 MHz, DMSO-d₆) 8.18 (s, 1H), 7.79 (s, 1H), 7.77-7.74 (m, 1H), 7.60 (s, 1H), 7.58-7.52 (m, 2H), 7.40-7.36 (m, 1H), 5.61-5.53 (m, 1H), 4.97-4.87 (m, 1H), 3.46 (s, 3H), 3.09-2.97 (m, 2H), 2.66 (s, 3H), 2.44- 2.36 (m, 2H), 1.98-1.80 (m, 2H), 1.65 (d, J = 7.1 Hz, 3H). |
| 23 | 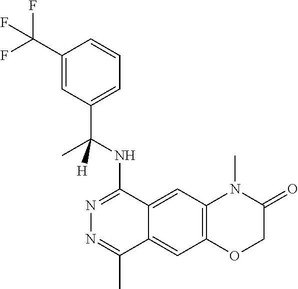 [SMILES: Cc1nnc(N[C@H](C)c2cccc(C(F)(F)F)c2)c2cc3c(cc12)OCC(=O)N3C] | UPLC-MS (ES⁺, Long acidic): 3.18 min, m/z 417.4 [M + H]⁺ (100%) ¹H NMR (400 MHz, DMSO-d₆) 8.02 (s, 1H), 7.78-7.76 (m, 1H), 7.76-7.71 (m, 1H), 7.58-7.52 (m, 3H), 7.47 (s, 1H), 5.63-5.51 (m, 1H), 4.86 (s, 2H), 3.50 (s, 3H), 2.56 (s, 3H), 1.63 (d, J = 7.1 Hz, 3H). |
| 24 | 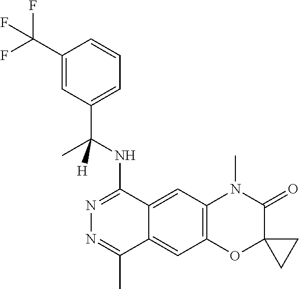 [SMILES: Cc1nnc(N[C@H](C)c2cccc(C(F)(F)F)c2)c2cc3c(cc12)OC1(CC1)C(=O)N3C] | UPLC-MS (ES⁺, Long acidic): 3.34 min, m/z 443.5 [M + H]⁺ (100%) ¹H NMR (400 MHz, DMSO-d₆): 8.04 (s, 1H), 7.79-7.73 (m, 2H), 7.60-7.52 (m, 3H), 7.42 (s, 1H), 5.62-5.53 (m, 1H), 3.53 (s, 3H), 2.55 (s, 3H), 1.63 (d, J = 7.1 Hz, 3H), 1.43-1.32 (m, 4H). |
| 25 | 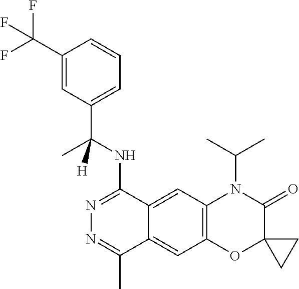 [SMILES: Cc1nnc(N[C@H](C)c2cccc(C(F)(F)F)c2)c2cc3c(cc12)OC1(CC1)C(=O)N3C(C)C] | UPLC-MS (ES⁺, Long acidic): 3.50 min, m/z 471.6 [M + H]⁺ (100%) ¹H NMR (400 MHz, DMSO-d₆) 8.05 (s, 1H), 7.79-7.77 (m, 1H), 7.76-7.73 (m, 1H), 7.60-7.54 (m, 3H), 7.46 (s, 1H), 5.63-5.54 (m, 1H), 4.78-4.69 (m, 1H), 2.55 (s, 3H), 1.68-1.60 (m, 9H), 1.32-1.22 (m, 4H). |
| 26 | 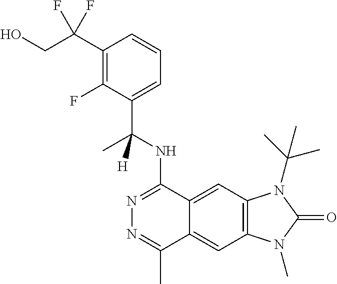 [SMILES: Cc1nnc(N[C@H](C)c2cccc(C(F)(F)CO)c2F)c2cc3c(cc12)n(C)c(=O)n3C(C)(C)C] | UPLC-MS (ES⁺, Long acidic): 3.04 min, m/z 488.6 [M + H]⁺ (97%) ¹H NMR (400 MHz, DMSO-d₆) d 8.20 (s, 1H), 7.60-7.55 (m, 2H), 7.50 (d, J = 7.2 Hz, 1H), 7.41-7.36 (m, 1H), 7.22-7.17 (m, 1H), 5.80-5.73 (m, 2H), 4.02-3.91 (m, 2H), 3.42 (s, 3H), 2.64 (s, 3H), 1.87 (s, 9H), 1.63 (d, J = 7.1 Hz, 3H) |
| 27 | 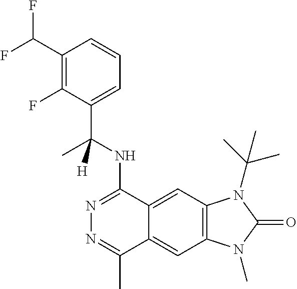 [SMILES: Cc1nnc(N[C@H](C)c2cccc(C(F)F)c2F)c2cc3c(cc12)n(C)c(=O)n3C(C)(C)C] | UPLC-MS (ES⁺, Long acidic): 3.29 min, m/z 458.6 [M + H]⁺ (100%) ¹H NMR (400 MHz, DMSO): 8.19 (s, 1H), 7.64-7.59 (m, 1H), 7.55 (s, 1H), 7.52 (d, J = 7.0 Hz, 1H), 7.48-7.44 (m, 1H), 7.40-7.12 (m, 2H), 5.77-5.73 (m, 1H), 3.42 (s, 3H), 2.64 (s, 3H), 1.87 (s, 9H), 1.64 (d, J = 7.0 Hz, 3H) |
| 28 | 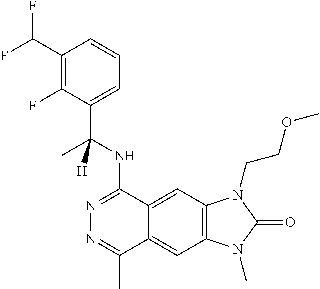 [SMILES: COCCn1c(=O)n(C)c2cc3c(C)nnc(N[C@H](C)c4cccc(C(F)F)c4F)c3cc21] | UPLC-MS (ES⁺, Long acidic): 3.14 min, m/z 460.4 [M + H]⁺ (92%) ¹H NMR (400 MHz, DMSO-d₆): 8.24 (s, 1H), 7.62-7.57 (m, 2H), 7.49-7.45 (m, 1H), 7.40-7.12 (m, 3H), 5.77- 5.70 (m, 1H), 4.18 (t, J = 5.7 Hz, 2H), 3.77 (t, J = 5.6 Hz, 2H), 3.49 (s, 3H), 3.28 (s, 3H), 2.66 (s, 3H), 1.64 (d, J = 7.0 Hz, 3H). |
| 29 | 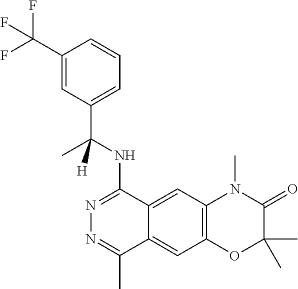 [SMILES: Cc1nnc(N[C@H](C)c2cccc(C(F)(F)F)c2)c2cc3c(cc12)OC(C)(C)C(=O)N3C] | UPLC-MS (ES⁺, Long acidic): 3.35 min, m/z 445.5 [M + H]⁺ (100%) ¹H NMR (400 MHz, DMSO-d₆): 8.03 (s, 1H), 7.79-7.77 (m, 1H), 7.77-7.73 (m, 1H), 7.59-7.54 (m, 3H), 7.48 (s, 1H), 5.62-5.54 (m, 1H), 3.52 (s, 3H), 2.57 (s, 3H), 1.63 (d, J = 7.0 Hz, 3H), 1.51 (s, 3H), 1.49 (s, 3H). |
| 30 | 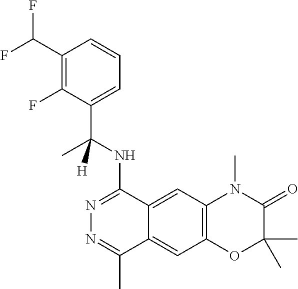 [SMILES: Cc1nnc(N[C@H](C)c2cccc(C(F)F)c2F)c2cc3c(cc12)OC(C)(C)C(=O)N3C] | UPLC-MS (ES⁺, Long acidic): 3.27 min, m/z 445.4 [M + H]⁺ (96%) ¹H NMR (400 MHz, DMSO-d₆): 8.08 (s, 1H), 7.65-7.56 (m, 2H), 7.50-7.45 (m, 2H), 7.26 (t, J = 54.4 Hz, 1H), 7.27- 7.22 (m, 1H), 5.77-5.69 (m, 1H), 3.53 (s, 3H), 2.56 (s, 3H), 1.63 (d, J = 7.0 Hz, 3H), 1.51 (s, 3H), 1.50 (s, 3H). |
| 31 | 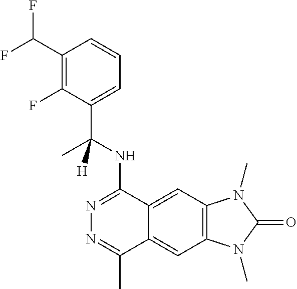 [SMILES: Cc1nnc(N[C@H](C)c2cccc(C(F)F)c2F)c2cc3c(cc12)n(C)c(=O)n3C] | UPLC-MS (ES⁺, Long acidic, UPLCMS-LONG-SQD): 2.81 min, m/z 416.4 [M + H] (100%) ¹H NMR (400 MHz, DMSO-d₆, 1HNMR) d 8.20 (s, 1H), 7.63-7.59 (m, 2H), 7.49-7.45 (m, 1H), 7.40- 7.11 (m, 3H), 5.77-5.69 (m, 1H), 3.52 (s, 3H), 3.48 (s, 3H), 2.66 (s, 3H), 1.63 (d, J = 7.0 Hz, 3H) |
| 32 | 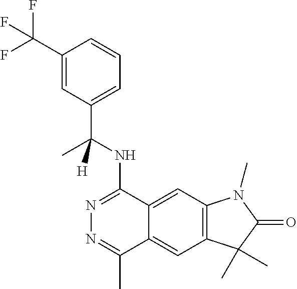 [SMILES: Cc1nnc(N[C@H](C)c2cccc(C(F)(F)F)c2)c2cc3c(cc12)C(C)(C)C(=O)N3C] | UPLC-MS (ES⁺, Long acidic): 3.09 min, m/z 429.6 [M + H]⁺ (98%) ¹H-NMR (400 MHz, DMSO-d₆,): 8.00 (s, 1H), 7.95 (s, 1H), 7.78 (s, 1H), 7.76- 7.72 (m, 1H), 7.58-7.51 (m, 2H), 7.47- 7.40 (m, 1H), 5.59-5.51 (m, 1H), 3.33 (s, 3H), 2.64 (s, 3H), 1.63 (d, J = 7.1 Hz, 3H), 1.40 (s, 3H), 1.39 (s, 3H). |
| 33 | 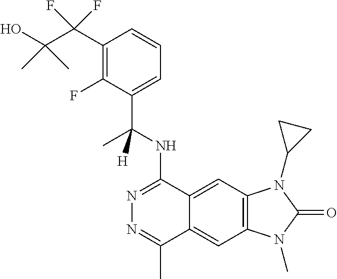 [SMILES: Cc1nnc(N[C@H](C)c2cccc(C(F)(F)C(C)(C)O)c2F)c2cc3c(cc12)n(C)c(=O)n3C1CC1] | UPLC-MS (ES⁺, Long acidic): 2.86 min, m/z 500.7 [M + H]⁺ (100%) ¹H NMR (400 MHz, DMSO-d₆,) d 8.18 (s, 1H), 7.58-7.51 (m, 2H), 7.47 (d, J = 7.3 Hz, 1H), 7.30-7.24 (m, 1H), 7.17-7.12 (m, 1H), 5.80-5.72 (m, 1H), 5.34 (s, 1H), 3.43 (s, 3H), 3.10- 3.03 (m, 1H), 2.64 (s, 3H), 1.61 (d, J = 7.0 Hz, 3H), 1.26-1.21 (6H, m), 1.19-1.13 (m, 2H), 1.08-0.98 (m, 2H) |
| 34 | 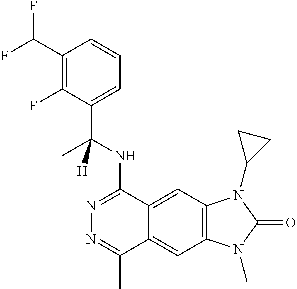 [SMILES: Cc1nnc(N[C@H](C)c2cccc(C(F)F)c2F)c2cc3c(cc12)n(C)c(=O)n3C1CC1] | UPLC-MS (ES⁺, Long acidic,): 2.94 min, m/z 442.4 [M + H]⁺ (100%) ¹H NMR (400 MHz, DMSO-d₆,) d 8.18 (s, 1H), 7.64-7.58 (m, 1H), 7.57 (s, 1H), 7.51 (d, J = 7.1 Hz, 1H), 7.47-7.42 (m, 1H), 7.39-7.11 (m, 2H), 5.78- 5.69 (m, 1H), 3.43 (s, 3H), 3.10-3.04 (m, 1H), 2.64 (s, 3H), 1.64 (d, J = 7.1 Hz, 3H), 1.19-1.17 (m, 2H), 1.09- 0.99 (m, 2H) |
| 35 | 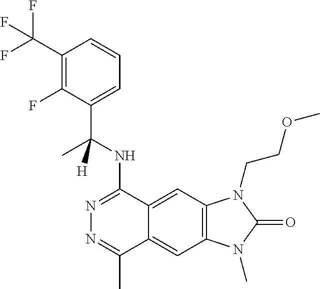 [SMILES: COCCn1c(=O)n(C)c2cc3c(C)nnc(N[C@H](C)c4cccc(C(F)(F)F)c4F)c3cc21] | UPLC-MS (ES⁺, Long acidic,): 3.07 min, m/z 478.4 [M + H]⁺ (100%) ¹H NMR (400 MHz, DMSO-d₆,) 8.24 (s, 1H), 7.76-7.70 (m, 1H), 7.64-7.59 (m, 2H), 7.41-7.37 (m, 1H), 7.34-7.28 (m, 1H), 5.75-5.70 (m, 1H), 4.20-4.16 (m, 2H), 3.79-3.75 (m, 2H), 3.49 (s, 3H), 3.28 (s, 3H), 2.66 (s, 3H), 1.65 (d, J = 7.1 Hz, 3H). |
| 36 | 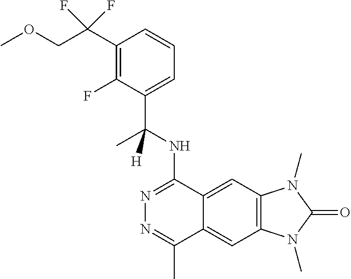 [SMILES: COCC(F)(F)c1cccc([C@@H](C)Nc2nnc(C)c3cc4c(cc23)n(C)c(=O)n4C)c1F] | UPLC-MS (ES⁺, Long acidic,): 2.88 min, m/z 460.4 [M + H]⁺ (98%) 1H NMR (400 MHz, DMSO-d₆,): 8.21 (s, 1H), 7.63 (s, 1H), 7.61-7.57 (m, 1H), 7.42-7.37 (m, 2H), 7.23-7.19 (m, 1H), 5.78-5.71 (m, 1H), 4.00 (t, J = 14.2 Hz, 2H), 3.52 (s, 3H), 3.49 (s, 3H), 3.37 (s, 3H), 2.67 (s, 3H), 1.62 (d, J = 7.0 Hz, 3H). |
| 37 | 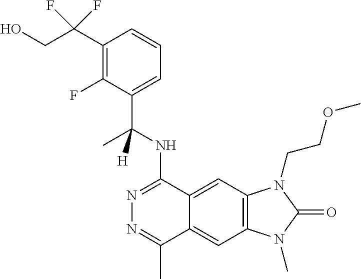 [SMILES: COCCn1c(=O)n(C)c2cc3c(C)nnc(N[C@H](C)c4cccc(C(F)(F)CO)c4F)c3cc21] | UPLC-MS (ES⁺, Long acidic,): 2.65 min, m/z 490.4 [M + H]⁺ (100%) ¹H NMR (400 MHz, DMSO-d₆) 8.25 (s, 1H), 7.62 (s, 1H), 7.59-7.54 (m, 1H), 7.41-7.36 (m, 1H), 7.32 (d, J = 7.4 Hz, 1H), 7.22-7.17 (m, 1H), 5.79- 5.72 (m, 2H), 4.18 (t, J = 5.6 Hz, 2H), 4.01-3.91 (m, 2H), 3.77 (t, J = 5.7 Hz, 2H), 3.49 (s, 3H), 3.28 (s, 3H), 2.66 (s, 3H), 1.63 (d, J = 7.0 Hz, 3H). |
| 38 | 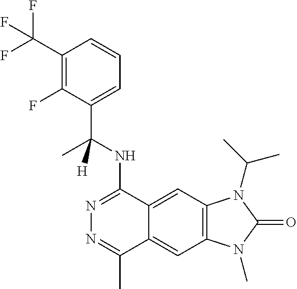 [SMILES: Cc1nnc(N[C@H](C)c2cccc(C(F)(F)F)c2F)c2cc3c(cc12)n(C)c(=O)n3C(C)C] | UPLC-MS (ES⁺, Long acidic,): 3.24 min, m/z 462.4 [M + H]⁺ (96%) 1H NMR (400 MHz, DMSO-d₆) 8.22 (s, 1H), 7.78-7.70 (m, 1H), 7.66-7.58 (m, 2H), 7.55-7.48 (brs, 1H), 7.36-7.30 (m, 1H), 5.78-5.70 (m, 1H), 4.79-4.72 (m, 1H), 3.47 (s, 3H), 2.66 (s, 3H), 1.67 (d, J 7.0 Hz, 3H), 1.62 (d, J = 6.9 Hz, 6H). |
| 39 | 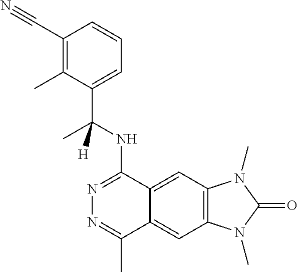 [SMILES: Cc1c(C#N)cccc1[C@@H](C)Nc1nnc(C)c2cc3c(cc12)n(C)c(=O)n3C] | UPLC-MS (ES⁺, Long acidic,): 2.68 min, m/z 387.2 [M + H]⁺ (100%) 1H NMR (400 MHz, DMSO-d6,): 8.36 (s, 1H), 7.81-7.79 (m, 1H), 7.61- 7.58 (m, 2H), 7.52-7.50 (m, 1H), 7.32- 7.28 (m, 1H), 5.63-5.56 (m, 1H), 3.51 (s, 3H), 3.49 (s, 3H), 2.69 (s, 3H), 2.64 (s, 3H), 1.56 (d, J = 7.0 Hz, 3H). |
| 40 | 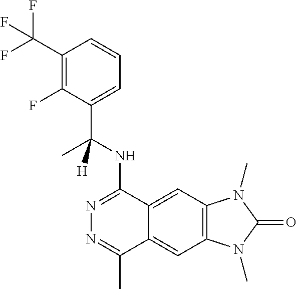 [SMILES: Cc1nnc(N[C@H](C)c2cccc(C(F)(F)F)c2F)c2cc3c(cc12)n(C)c(=O)n3C] | UPLC-MS (ES⁺, Long acidic): 3.00 min, m/z 434.4 [M + H]⁺ (100%) ¹H NMR (400 MHz, DMSO-d₆,) d 8.19 (s, 1H), 7.76-7.72 (m, 1H), 7.63-7.58 (m, 2H), 7.41 (d, J = 7.3 Hz, 1H), 7.32- 7.27 (m, 1H), 5.76-5.68 (m, 1H), 3.51 (s, 3H), 3.48 (s, 3H), 2.65 (s, 3H), 1.64 (d, J = 7.0 Hz, 3H) |
| 41 | 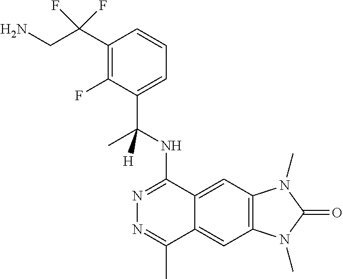 [SMILES: Cc1nnc(N[C@H](C)c2cccc(C(F)(F)CN)c2F)c2cc3c(cc12)n(C)c(=O)n3C] | UPLC-MS (ES⁺, Long acidic,): 1.97 min, m/z 445.2 [M + H]⁺ (100%) 1H NMR (400 MHz, DMSO-d6,) d 8.20 (s, 1H), 7.62 (s, 1H), 7.58-7.53 (m, 1H), 7.39-7.35 (m, 1H), 7.33 (d, J = 7.5 Hz, 1H), 7.20-7.16 (m, 1H), 5.79-5.71 (m, 1H), 3.51 (s, 3H), 3.48 (s, 3H), 3.25 (t, J = 15.5 Hz, 2H), 2.66 (s, 3H), 1.61 (d, J = 7.0 Hz, 3H) |
| 42 | 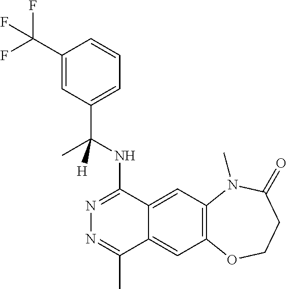 [SMILES: Cc1nnc(N[C@H](C)c2cccc(C(F)(F)F)c2)c2cc3c(cc12)OCCC(=O)N3C] | UPLC-MS (ES⁺, Long acidic,): 2.91 min, m/z 431.4 [M + H]⁺ (97%) 1H NMR (400 MHz, DMSO-d6, ): 8.41 (s, 1H), 7.77 (s, 1H), 7.76-7.71 (m, 1H), 7.67 (s, 1H), 7.61-7.50 (m, 3H), 5.55 (quint, J = 7.0 Hz, 1H), 4.64-4.52 (m, 2H), 3.44 (s, 3H), 2.73-2.61 (m, 2H), 2.59 (s, 3H), 1.62 (d, J = 7.0 Hz, 3H) |
| 43 | 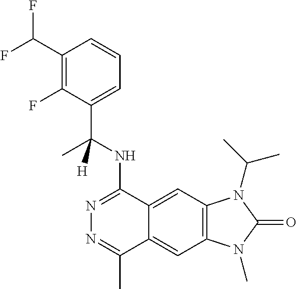 [SMILES: Cc1nnc(N[C@H](C)c2cccc(C(F)F)c2F)c2cc3c(cc12)n(C)c(=O)n3C(C)C] | UPLC-MS (ES⁺, Long acidic,): 3.08 min, m/z 444.5 [M + H]⁺ (100%) ¹H NMR (400 MHz, DMSO-d₆,) 8.20 (s, 1H), 7.64-7.56 (m, 2H), 7.50-7.40 (m, 2H), 7.25 (t, J = 54.6 Hz, 1H), 7.25- 7.21 (m, 1H), 5.78-5.69 (m, 1H), 4.78- 4.70 (m, 1H), 3.46 (s, 3H), 2.64 (s, 3H), 1.66-1.58 (m, 9H). |
| 44 | 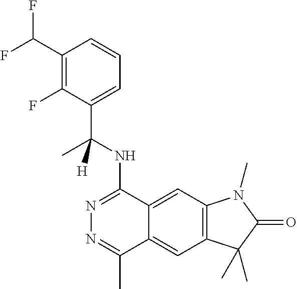 [SMILES: Cc1nnc(N[C@H](C)c2cccc(C(F)F)c2F)c2cc3c(cc12)C(C)(C)C(=O)N3C] | UPLC-MS (ES⁺, Long acidic,): 2.98 min, m/z 429.6 [M + H]⁺ (100%) ¹H-NMR (400 MHz, DMSO-d₆,): 8.00- 7.98 (m, 2H), 7.61-7.56 (m, 1H), 7.48-7.41 (m, 2H), 7.39-7.11 (m, 2H), 5.76-5.67 (m, 1H), 3.33 (s, 3H), 2.63 (s, 3H), 1.63 (d, J = 7.0 Hz, 3H), 1.40 (s, 3H), 1.39 (s, 3H) |
| 45 | 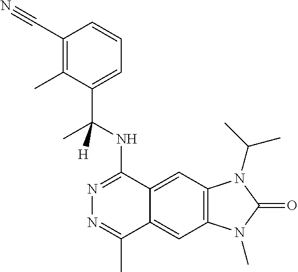 [SMILES: Cc1c(C#N)cccc1[C@@H](C)Nc1nnc(C)c2cc3c(cc12)n(C(C)C)c(=O)n3C] | UPLC-MS (ES⁺, Long acidic): 2.96 min, m/z 415.4 [M + H]⁺ (100%). 1H-NMR (400 MHz, DMSO-d₆): 8.18 (s, 1H), 7.76 (dd, J = 7.8, 1.0 Hz, 1H), 7.63-7.58 (m, 2H), 7.55-7.45 (m, 1H), 7.35-7.29 (m, 1H), 5.62-5.57 (m, 1H), 4.78-4.69 (m, 1H), 3.45 (s, 3H), 2.69 (s, 3H), 2.65 (s, 3H), 1.62-1.56 (m, 9H). |
| 46 | 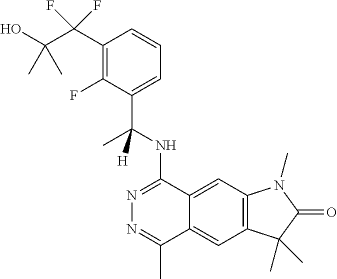 [SMILES: Cc1nnc(N[C@H](C)c2cccc(C(F)(F)C(C)(C)O)c2F)c2cc3c(cc12)C(C)(C)C(=O)N3C] | UPLC-MS (ES⁺, Long acidic,): 2.95 min, m/z 487.5 [M + H]⁺ (100%) ¹H-NMR (400 MHz, DMSO-d₆,): 7.99 (s, 2H), 7.54-7.49 (m, 1H), 7.40-7.36 (m, 1H), 7.30-7.25 (m, 1H), 7.17-7.12 (m, 1H), 5.77-5.70 (m, 1H), 5.34 (s, 1H), 3.33 (s, 3H), 2.63 (s, 3H), 1.60 (d, J = 7.0 Hz, 3H), 1.40 (s, 3H), 1.39 (s, 3H), 1.24 (s, 3H), 1.23 (s, 3H). |
| 47 | 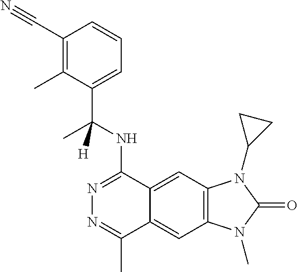 [SMILES: Cc1c(C#N)cccc1[C@@H](C)Nc1nnc(C)c2cc3c(cc12)n(C1CC1)c(=O)n3C] | UPLC-MS (ES⁺, Long acidic,): 2.81 min, m/z 413.6 [M + H]⁺ (100%) 1H NMR (400 MHz, DMSO-d₆,) d 8.14 (s, 1H), 7.80-7.77 (m, 1H), 7.60- 7.58 (m, 1H), 7.56-7.52 (m, 2H), 7.33- 7.28 (m, 1H), 5.63-5.56 (m, 1H), 3.42 (s, 3H), 3.09-3.03 (m, 1H), 2.69 (s, 3H), 2.64 (s, 3H), 1.57 (d, J = 7.0 Hz, 3H), 1.20-1.15 (m, 2H), 1.08-0.97 (m, 2H). |
| 48 | 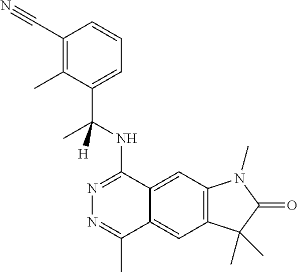 [SMILES: Cc1c(C#N)cccc1[C@@H](C)Nc1nnc(C)c2cc3c(cc12)N(C)C(=O)C3(C)C] | UPLC-MS (ES⁺, Long acidic,): 2.84 min, m/z 400.6 [M + H]⁺ (98%) 1H NMR (400 MHz, DMSO-d₆,) d 7.98 (s, 1H), 7.95 (s, 1H), 7.77-7.74 (m, 1H), 7.62-7.59 (m, 1H), 7.46 (d, J = 6.8 Hz, 1H), 7.33-7.28 (m, 1H), 5.63-5.55 (m, 1H), 2.68 (s, 3H), 2.62 (s, 3H), 1.56 (d, J = 7.0 Hz, 3H), 1.39 (s, 3H), 1.37 (s, 3H). CH₃ is under solvent peak. |
| 49 | 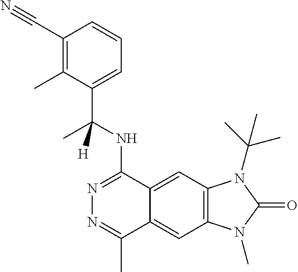 [SMILES: Cc1c(C#N)cccc1[C@@H](C)Nc1nnc(C)c2cc3c(cc12)n(C(C)(C)C)c(=O)n3C] | UPLC-MS (ES⁺, Long acidic,): 3.10 min, m/z 429.6 [M + H]⁺ (98%) 1H NMR (400 MHz, DMSO-d₆,) d 8.15 (s, 1H), 7.78 (dd, J = 8.1, 1.2 Hz, 1H), 7.59 (dd, J = 8.0, 1.3 Hz, 1H), 7.55- 7.51 (m, 2H), 7.33-7.28 (m, 1H), 5.63-5.55 (m, 1H), 3.41 (s, 3H), 2.69 (s, 3H), 2.63 (s, 3H), 1.86 (s, 9H), 1.57 (d, J = 7.0 Hz, 3H) |
| 50 | 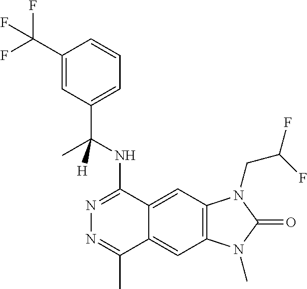 [SMILES: Cc1nnc(N[C@H](C)c2cccc(C(F)(F)F)c2)c2cc3c(cc12)n(C)c(=O)n3CC(F)F] | UPLC-MS (ES⁺, Long acidic,): 3.10 min, m/z 466.4 [M + H]⁺ (100%) 1H NMR (400 MHz, DMSO-d₆,): 8.25 (s, 1H), 7.78 (s, 1H), 7.75-7.72 (m, 1H), 7.65 (s, 1H), 7.55-7.53 (m, 2H), 7.30-7.27 (m, 1H), 6.64-6.37 (m, 1H), 5.61-5.53 (m, 1H), 4,47-4.36 (m, 2H), 3.50 (s, 3H), 2.66 (s, 3H), 1.63 (d, J = 7.2 Hz, 3H). |
| 51 | 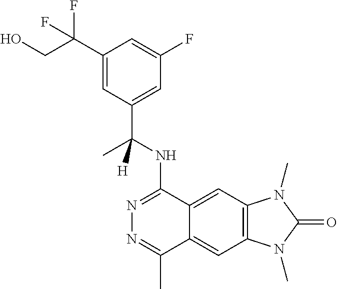 [SMILES: Cc1nnc(N[C@H](C)c2cc(F)cc(C(F)(F)CO)c2)c2cc3c(cc12)n(C)c(=O)n3C] | UPLC-MS (ES⁺, Long acidic,): 2.60 min, m/z 446.4 [M + H]⁺ (100%) 1H NMR (400 MHz, DMSO-d₆, ): 8.14 (s, 1H), 7.61 (s, 1H), 7.47 (s, 1H), 7.42- 7.31 (m, 2H), 7.21-7.13 (m, 1H), 5.64 (t, J = 6.3 Hz, 1H), 5.54 (quint, J = 7.1 Hz, 1H), 3.90-3.77 (m, 2H), 3.50 (s, 3H), 3.47 (s, 3H), 2.67 (s, 3H), 1.61 (d, J = 7.0 Hz, 3H) |
| 52 | 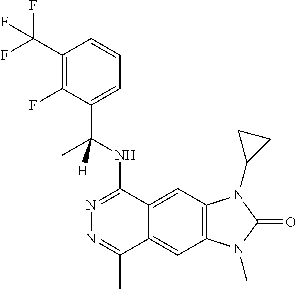 [SMILES: Cc1nnc(N[C@H](C)c2cccc(C(F)(F)F)c2F)c2cc3c(cc12)n(C)c(=O)n3C1CC1] | UPLC-MS (ES⁺, Long acidic,): 3.12 min, m/z 460.4 [M + H]⁺ (100%) 1H NMR (400 MHz, DMSO-d₆,) d 8.18 (s, 1H), 7.79-7.72 (m, 1H), 7.63- 7.55 (m, 3H), 7.33-7.28 (m, 3H), 5.77- 5.70 (m, 1H), 3.44 (s, 3H), 3.11-3.05 (m, 1H), 2.65 (s, 3H), 1.66 (d, J = 7.1 Hz, 3H), 1.20-1.18 (m, 2H), 1.09- 0.99 (m, 2H) |
| 53 | 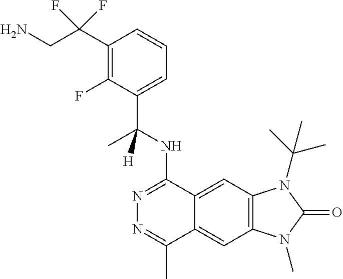 [SMILES: Cc1nnc(N[C@H](C)c2cccc(C(F)(F)CN)c2F)c2cc3c(cc12)n(C)c(=O)n3C(C)(C)C] | UPLC-MS (ES⁺, Long acidic,): 2.33 min, m/z 487.4 [M + H]⁺ (100%) 1H NMR (400 MHz, DMSO-d6,): 8.19 (s, 1H), 7.59-7.53 (m, 2H), 7.49 (d, J = 7.2 Hz, 1H), 7.39-7.34 (m, 1H), 7.22- 7.15 (m, 1H), 5.80-5.72 (m, 1H), 3.42 (s, 3H), 3.25 (t, J = 15.3 Hz, 2H), 2.64 (s, 3H), 1.86 (s, 9H), 1.62 (d, J = 7.0 Hz, 3H). Exchangeable protons not observed (—NH2). |
| 54 | 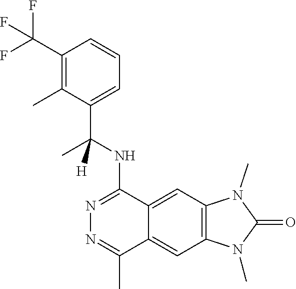 [SMILES: Cc1c([C@@H](C)Nc2nnc(C)c3cc4c(cc23)n(C)c(=O)n4C)cccc1C(F)(F)F] | UPLC-MS (ES⁺, Long acidic,): 3.10 min, m/z 430.5 [M + H]⁺ (97%). 1H NMR (400 MHz, DMSO-d₆,): 8.18 (s, 1H), 7.77 (d, J = 7.8 Hz, 1H), 7.59 (s, 1H), 7.53-7.49 (m, 1H), 7.39 (d, J = 7.0 Hz, 1H), 7.34-7.27 (m, 1H), 5.75- 5.67 (m, 1H), 3.50 (s, 3H), 3.47 (s, 3H), 2.65 (s, 3H), 2.59 (br s, 3H), 1.57 (d, J = 7.0 Hz, 3H). |
| 55 | 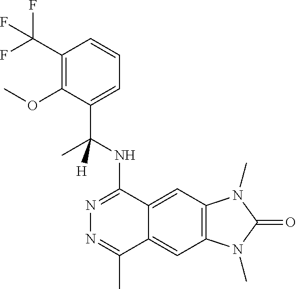 [SMILES: COc1c([C@@H](C)Nc2nnc(C)c3cc4c(cc23)n(C)c(=O)n4C)cccc1C(F)(F)F] | UPLC-MS (ES⁺, Long acidic,): 3.04 min, m/z 446.2 [M + H]⁺ (100%) 1H NMR (400 MHz, DMSO-d6): 8.24 (s, 1H), 7.79-7.72 9m, 1H), 7.65 (s, 1H), 7.55-7.42 (m, 2H), 7.27-7.17 (m, 1H), 5.74 (quint, J = 6.9 Hz, 1H), 4.14 (s, 3H), 3.52 (s, 3H), 3.48 (s, 3H), 2.67 (s, 3H), 1.57 (d, J = 6.9 Hz, 3H) |
| 56 | 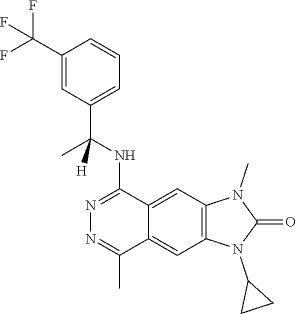 [SMILES: Cc1nnc(N[C@H](C)c2cccc(C(F)(F)F)c2)c2cc3c(cc12)n(C1CC1)c(=O)n3C] | UPLC-MS (ES⁺, Long acidic,): 3.08 min, m/z 442.3 [M + H]⁺ (97%) 1H NMR (400 MHz, DMSO-d6,): 8.14 (s, 1H), 7.76 (s, 1H), 7.75-7.72 (m, 1H), 7.57-7.49 (m, 3H), 7.44-7.38 (m, 1H), 5.57-5.51 (m, 1H), 3.46 (s, 3H), 3.05-2.99 (m, 1H), 2.67 (s, 3H), 1.62 (d, J = 7.1 Hz, 3H), 1.15-1.11 (m, 2H), 0.99- 0.95 (m, 2H). |
| 57 | 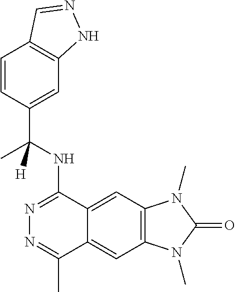 [SMILES: Cc1nnc(N[C@H](C)c2ccc3cn[nH]c3c2)c2cc3c(cc12)n(C)c(=O)n3C] | UPLC-MS (ES⁺, Long acidic,): 2.40 min, m/z 388.3 [M + H]⁺ (100%) 1H NMR (400 MHz, DMSO-d6): 12.86 (1H, s), 8.24 (1H, s), 7.97 (1H, s), 7.67 (1H, d, J = 8.4 Hz), 7.63 (1H, s), 7.52 (1H, s), 7.43 (1H, br s), 7.24 (1H, dd, J = 1.3, 8.4 Hz), 5.62 (1H, m), 3.51 (3H, s), 3.48 (3H, s), 2.66 (3H, s), 1.67 (3H, d, J = 7.0 Hz). |
| 58 | 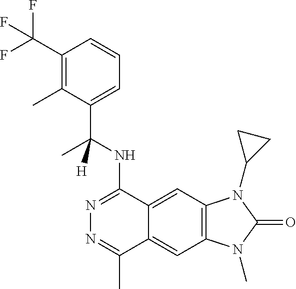 [SMILES: Cc1c([C@@H](C)Nc2nnc(C)c3cc4c(cc23)n(C2CC2)c(=O)n4C)cccc1C(F)(F)F] | UPLC-MS (ES⁺, Long acidic,): 3.24 min, m/z 456.3 [M + H]⁺ (100%) 1H NMR (400 MHz, DMSO-d₆,) d 8.17 (s, 1H), 7.79 (d, J = 7.8 Hz, 1H), 7.57-7.55 (m, 2H), 7.51 (d, J = 7.8 Hz, 1H), 7.33-7.28 (m, 1H), 5.75-5.67 (m, 1H), 3.42 (s, 3H), 3.10-3.03 (m, 1H), 2.64 (s, 3H), 2.59 (s, 3H), 1.58 (d, J = 7.0 Hz, 3H), 1.19-1.17 (m, 2H), 1.08-0.99 (m, 2H) |
| 59 | 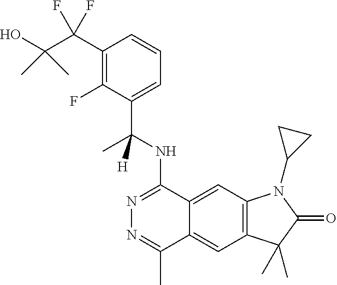 [SMILES: Cc1nnc(N[C@H](C)c2cccc(C(F)(F)C(C)(C)O)c2F)c2cc3c(cc12)C(C)(C)C(=O)N3C1CC1] | UPLC-MS (ES⁺, Long acidic, 3.08 min, m/z 513.3 [M + H]⁺ and 535.3 [M + Na]⁺ (100%) ¹H-NMR (400 MHz, DMSO-d₆,): 8.02 (s, 1H), 7.97 (s, 1H), 7.55-7.12 (m, 2H), 7.30-7.25 (m, 1H), 7.17-7.48 (m, 1H), 5.78-5.70 (m, 1H), 5.35 (s, 1H), 2.90-2.84 (m, 1H), 2.62 (s, 3H), 1.61 (d, J = 7.0 Hz, 3H), 1.37-1.35 (m, 6H), 1.24 (s, 3H), 1.23 (s, 3H), 1.19-1.14 (m, 2H), 0.99-0.92 (m, 1H), 0.92-0.83 (m, 1H) |
| 60 | 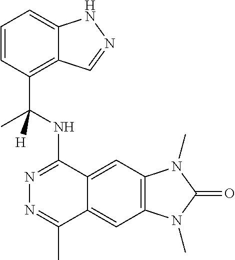 [SMILES: Cc1nnc(N[C@H](C)c2cccc3[nH]ncc23)c2cc3c(cc12)n(C)c(=O)n3C] | UPLC-MS (ES⁺, Long acidic,): 2.38 min, m/z 388.1 [M + H]⁺ (100%) 1H NMR (400 MHz, DMSO-d₆,) δ 13.00 (s, 1H), 8.25 (s, 1H), 8.22 (s, 1H), 7.59 (s, 1H), 7.37-7.32 (m, 2H), 7.28-7.21 (m, 1H), 7.12 (d, J = 7.1 Hz, 1H), 5.96-5.89 (m, 1H), 3.51 (s, 3H), 3.47 (s, 3H), 2.64 (s, 3H), 1.73 (d, J = 7.0 Hz, 3H). |
| 61 | 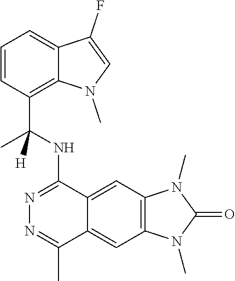 [SMILES: Cc1nnc(N[C@H](C)c2cccc3c(F)cn(C)c23)c2cc3c(cc12)n(C)c(=O)n3C] | UPLC-MS (ES⁺, Long acidic): 3.00 min, m/z 419.3 [M + H]⁺ (98%) ¹H-NMR (400 MHz, DMSO-d₆,): 8.20 (s, 1H), 7.59 (s, 1H), 7.41-7.34 (m, 3H), 7.30 (d, J = 2.5 Hz, 1H), 7.02-6.97 (m, 1H), 6.39-6.31 (m, 1H), 4.10 (s, 3H), 3.49 (s, 3H), 3.47 (s, 3H), 2.65 (s, 3H), 1.69 (d, J = 6.8 Hz, 3H) |
| 62 | 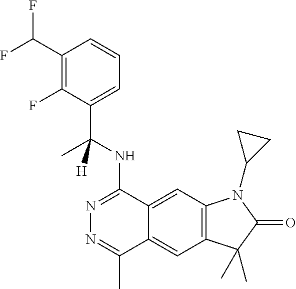 [SMILES: Cc1nnc(N[C@H](C)c2cccc(C(F)F)c2F)c2cc3c(cc12)C(C)(C)C(=O)N3C1CC1] | UPLC-MS (ES⁺, Long acidic,): 3.18 min, m/z 455.3 [M + H]⁺ (100%) ¹H-NMR (400 MHz, DMSO-d₆,): 8.01 (s, 1H), 7.97 (s, 1H), 7.62-7.57 (m, 1H), 7.54 (d, J = 7.1 Hz, 1H), 7.48-7.43 (m, 1H), 7.25 (t, J = 54.6 Hz, 1H), 7.24- 7.20 (m, 1H), 5.77-5.68 (m, 1H), 2.90- 2.83 (m, 1H), 2.62 (s, 3H), 1.64 (d, J = 7.1 Hz, 3H), 1.36 (s, 3H), 1.35 (s, 3H), 1.20-1.12 (m, 2H), 0.99-0.83 (m, 2H). |
| 63 | 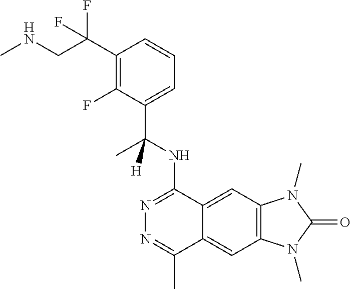 [SMILES: CNCC(F)(F)c1cccc([C@@H](C)Nc2nnc(C)c3cc4c(cc23)n(C)c(=O)n4C)c1F] | UPLC-MS (ES⁺, Long acidic, 2.01 min, m/z 459.3 [M + H]⁺ (100%) 1H-NMR (400 MHz, DMSO-d₆,): 8.21 (s, 1H), 7.63 (s, 1H), 7.58-7.53 (m, 1H), 7.40-7.33 (m, 2H), 7.20-7.15 (m, 1H), 5.79-5.71 (m, 1H), 3.51 (s, 3H), 3.48 (s, 3H), 3.29-3.21 (m, 2H), 2.66 (s, 3H), 2.31 (s, 3H), 1.61 (d, J = 7.0 Hz, 3H). Exchangeable proton not observed. |
| 64 | 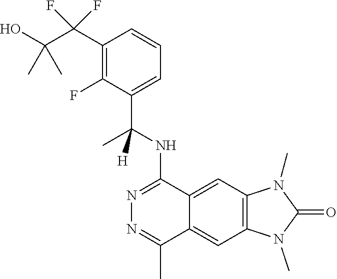 [SMILES: Cc1nnc(N[C@H](C)c2cccc(C(F)(F)C(C)(C)O)c2F)c2cc3c(cc12)n(C)c(=O)n3C] | UPLC-MS (ES⁺, Long acidic): 2.81 min, m/z 456.3 [M + H]⁺ (100%) 1H NMR (400 MHz, DMSO-d₆) 8.17 (s, 1H), 7.60 (s, 1H), 7.56-7.52 (m, 2H), 7.39-7.34 (m, 1H), 7.31-7.25 (m, 2H), 5.60-5.52 (m, 1H), 5.21 (s, 1H), 3.50 (s, 3H), 3.48 (s, 3H), 2.66 (s, 3H), 1.62 (d, J = 7.0 Hz, 3H), 1.09 (d, J = 14.8 Hz, 6H) |
| 65 | 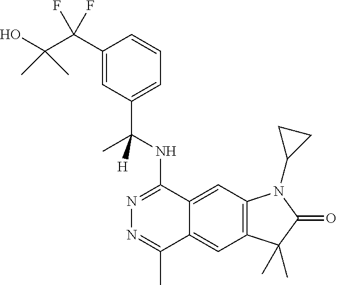 [SMILES: Cc1nnc(N[C@H](C)c2cccc(C(F)(F)C(C)(C)O)c2)c2cc3c(cc12)C(C)(C)C(=O)N3C1CC1] | UPLC-MS (ES⁺, Long acidic,): 3.05 min, m/z 495.3 [M + H]⁺ (98%) ¹H-NMR (400 MHz, DMSO-d₆,): 7.98 (s, 1H), 7.96 (s, 1H), 7.58-7.47 (m, 3H), 7.39-7.32 (m, 1H), 7.31-7.27 (m, 1H), 5.61-5.52 (m, 1H), 5.21 (s, 1H), 2.88-2.82 (m, 1H), 2.63 (s, 3H), 1.63 (d, J = 7.0 Hz, 3H), 1.35 (s, 6H), 1.17- 1.13 (m, 2H), 1.12 (s, 3H), 1.09 (s, 3H), 0.95-0.85 (m, 2H). |
| 66 | 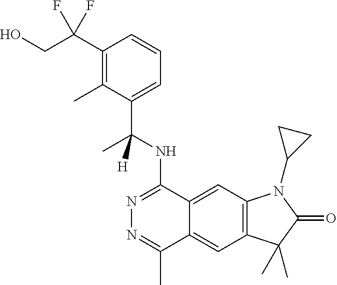 [SMILES: Cc1c([C@@H](C)Nc2nnc(C)c3cc4c(cc23)N(C2CC2)C(=O)C4(C)C)cccc1C(F)(F)CO] | UPLC-MS (ES⁺, Long acidic): 2.96 min, m/z 481.3 [M + H]⁺ (100%) ¹H-NMR (400 MHz, DMSO-d₆): 8.02 (s, 1H), 7.97 (s, 1H), 7.63 (d, J = 7.9 Hz, 1H), 7.55 (br s, 1H), 7.33-7.30 (m, 1H), 7.22-7.17 (m, 1H), 5.75-5.68 (m, 1H), 5.67 (t, J = 6.4 Hz, 1H), 3.97-3.86 (m, 2H), 2.90-2.84 (m, 1H), 2.63 (s, 3H), 2.54 (s, 3H,), 1.56 (d, J = 6.9 Hz, 3H), 1.35 (s, 3H), 1.35 (s, 3H), 1.19-1.13 (m, 2H), 1.00-0.84 (m, 2H). |
| 67 | 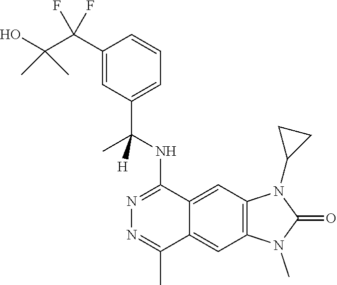 [SMILES: Cc1nnc(N[C@H](C)c2cccc(C(F)(F)C(C)(C)O)c2)c2cc3c(cc12)n(C)c(=O)n3C1CC1] | UPLC-MS (ES⁺, Long acidic): 2.89 min, m/z 482.3 [M + H]+ (100%) 1H NMR (400 MHz, DMSO-d₆,) d 8.15 (s, 1H), 7.57-7.52 (m, 3H), 7.44 (d, J = 7.5 Hz, 1H), 7.39-7.33 (m, 1H), 7.30-7.27 (m, 1H), 5.63-5.54 (m, 1H), 5.19 (s, 1H), 3.43 (s, 3H), 3.08- 3.01 (m, 1H), 2.65 (s, 3H), 1.63 (d, J = 7.0 Hz, 3H), 1.20-1.14 (m, 2H), 1.11 (s, 3H), 1.07 (s, 3H), 1.03-0.99 (m, 2H) |
| 68 | 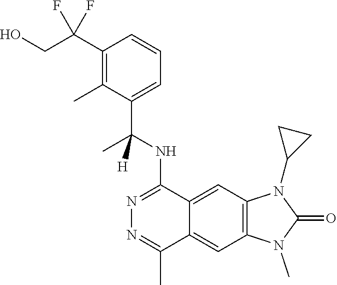 [SMILES: Cc1c([C@@H](C)Nc2nnc(C)c3cc4c(cc23)n(C2CC2)c(=O)n4C)cccc1C(F)(F)CO] | UPLC-MS (ES⁺, Long acidic,): 2.78 min, m/z 468.4 [M + H]⁺ (100%) 1H NMR (400 MHz, DMSO-d6,) d 8.17 (s, 1H), 7.65 (d, J = 7.6 Hz, 1H), 7.55 (s, 1H), 7.47 (d, J = 7.2 Hz, 1H), 7.32-7.30 (m, 1H), 7.22-7.16 (m, 1H), 5.79-5.71 (m, 1H), 5.67 (t, J = 6.4 Hz, 1H), 3.91 (td, J = 14.6, 6.5 Hz, 2H), 3.42 (s, 3H), 3.09-3.03 (m, 1H), 2.64 (s, 3H), 2.55 (s, 3H), 1.56 (d, J = 7.0 Hz, 3H), 1.20-1.16 (m, 2H), 1.09-0.96 (m, 2H) |
| 69 | 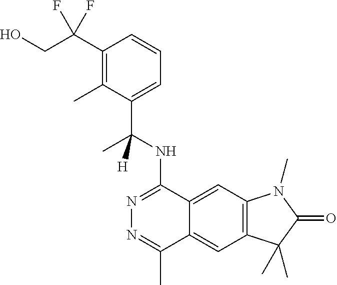 [SMILES: Cc1c([C@@H](C)Nc2nnc(C)c3cc4c(cc23)N(C)C(=O)C4(C)C)cccc1C(F)(F)CO] | UPLC-MS (ES⁺, Long acidic): 2.83 min, m/z 455.3 [M + H]⁺ (100%) ¹H-NMR (400 MHz, DMSO-d₆): 7.99- 7.96 (m, 2H), 7.60 (d, J = 7.1 Hz, 1H), 7.38 (d, J = 7.1 Hz, 1H), 7.31 (d, J = 7.6 Hz, 1H), 7.23-7.17 (m, 1H), 5.77- 5.70 (m, 1H), 5.67 (t, J = 6.4 Hz, 1H), 3.97-3.85 (m, 2H), 2.62 (s, 3H), 2.54 (s, 3H), 1.55 (d, J = 6.8 Hz, 3H), 1.39 (s, 3H), 1.38 (s, 3H). CH₃ under water peak. |
| 70 | 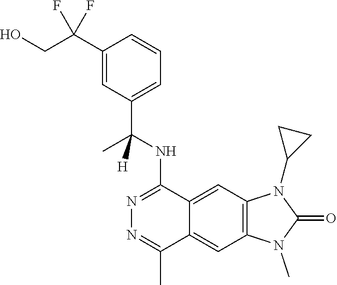 [SMILES: Cc1nnc(N[C@H](C)c2cccc(C(F)(F)CO)c2)c2cc3c(cc12)n(C)c(=O)n3C1CC1] | UPLC-MS (ES⁺, Long acidic,): 2.68 min, m/z 454.3 [M + H]⁺ (100%) 1H NMR (400 MHz, DMSO-d₆,) d 8.15 (s, 1H), 7.62 (s, 1H), 7.59-7.55 (m, 2H), 7.47 (d, J = 7.5 Hz, 1H), 7.43- 7.37 (m, 1H), 7.35-7.31 (m, 1H), 5.63- 5.55 (m, 2H), 3.81 (td, J = 14.3, 6.4 Hz, 2H), 3.43 (s, 3H), 3.09-3.02 (m, 1H), 2.65 (s, 3H), 1.63 (d, J = 7.1 Hz, 3H), 1.19-1.15 (m, 2H), 1.08-0.96 (m, 2H) |
| 72 | 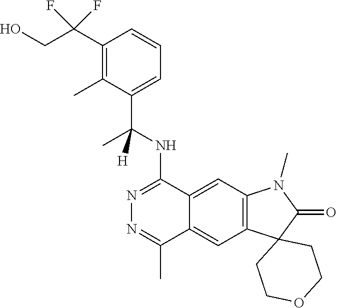 [SMILES: Cc1c([C@@H](C)Nc2nnc(C)c3cc4c(cc23)N(C)C(=O)C42CCOCC2)cccc1C(F)(F)CO] | UPLC-MS (ES⁺, Long acidic,): 2.78 min, m/z 497.3 [M + H]⁺ (100%) ¹H-NMR (400 MHz, DMSO-d₆,): 8.03 (s, 1H), 7.98 (s, 1H), 7.62-7.59 (m, 1H), 7.42-7.37 (m, 1H), 7.34-7.29 (m, 1H), 7.22-7.16 (m, 1H), 5.76-5.70 (m, 1H), 5.67 (t, J = 6.5 Hz, 1H), 4.16-4.07 (m, 2H), 3.96-3.82 (m, 4H), 2.65 (s, 3H), 2.06-1.93 (m, 2H), 1.80-1.71 (m, 2H), 1.55 (d, J = 6.9 Hz, 3H)- 3H missing under water peak and 3H under DMSO peak. |
| 73 | 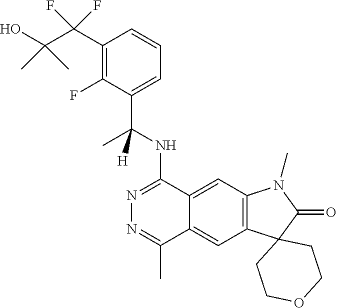 [SMILES: Cc1nnc(N[C@H](C)c2cccc(C(F)(F)C(C)(C)O)c2F)c2cc3c(cc12)C1(CCOCC1)C(=O)N3C] | UPLC-MS (ES⁺, Long acidic): 2.94 min, m/z 529.3 [M + H]⁺ (100%) ¹H-NMR (400 MHz, DMSO-d₆): 8.05 (s, 1H), 7.99 (s, 1H), 7.54-7.48 (m, 1H), 7.39 (d, J = 7.4 Hz, 1H), 7.30-7.25 (m, 1H), 7.17-7.12 (m, 1H), 5.77-5.69 (m, 1H), 5.34 (s, 1H), 4.16-4.08 (m, 2H), 3.92-3.83 (m, 2H), 3.33 (s, 3H), 2.65 (s, 3H), 2.08-1.96 (m, 2H), 1.80- 1.72 (m, 2H), 1.60 (d, J = 7.0 Hz, 3H), 1.25-1.22 (m, 6H). |
| 74 | 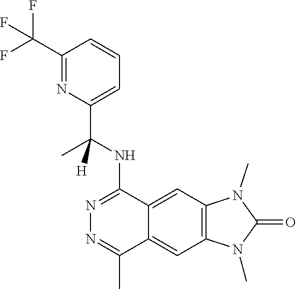 [SMILES: Cc1nnc(N[C@H](C)c2cccc(C(F)(F)F)n2)c2cc3c(cc12)n(C)c(=O)n3C] | UPLC-MS (ES⁺, Long acidic,): 2.76 min, m/z 417.2 [M + H]⁺ (100%) 1H NMR (400 MHz, Methanol-d₄,) δ 8.55 (s, 1H), 8.15 (s, 1H), 7.94-7.87 (m, 1H), 7.72 (d, J = 7.9 Hz, 1H), 7.66 (s, 1H), 7.62 (d, J = 7.7 Hz, 1H), 5.61-5.54 (m, 1H), 3.61 (s, 3H), 3.58 (s, 3H), 2.75 (s, 3H), 1.76 (d, J = 7.0 Hz, 3H). |
| 75 | 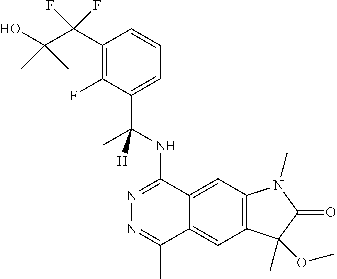 [SMILES: COC1(C)C(=O)N(C)c2cc3c(N[C@H](C)c4cccc(C(F)(F)C(C)(C)O)c4F)nnc(C)c3cc21] | UPLC-MS (ES⁺, Long acidic): 2.91 min, m/z 503.3 [M + H]⁺ (100%) 1H NMR (400 MHz, DMSO-d₆): 8.04 (s, 1H), 7.97 (s, 1H), 7.57-7.48 (m, 1H), 7.49-7.42 (m, 1H), 7.32-7.23 (m, 1H), 7.20-7.10 (m, 1H), 5.73 (quintet, J = 7.1 Hz, 1H), 5.35 (s, 1H), 3.35 (s, 3H), 2.92 (d, J = 2.6 Hz, 3H), 2.65 (s, 3H), 1.61 (d, J = 7.0 Hz, 3H), 1.54 (s, 3H), 1.24 (s, 3H), 1.23 (s, 6H) |
| 76 | 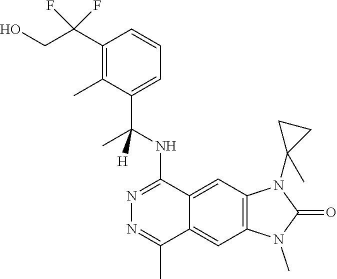 | UPLC-MS (ES⁺, Long acidic): 2.91 min, m/z 4682.3 [M + H]⁺ (100%) 1H NMR (400 MHz, DMSO-d₆d 8.15 (s, 1H), 7.65 (d, J = 7.6 Hz, 1H), 7.56 (s, 1H), 7.49 (d, J = 7.0 Hz, 1H), 7.31 (d, J = 7.8 Hz, 1H), 7.23-7.17 (m, 1H), 5.80-5.72 (m, 1H), 5.67 (t, J = 6.5 Hz, 1H), 3.97-3.85 (m, 2H), 3.42 (s, 3H), 2.64 (s, 3H), 2.55 (s, 3H), 1.58 (d, J = 6.9 Hz, 3H), 1.53 (s, 3H), 1.16-1.09 (m, 4H). |
| 77 | 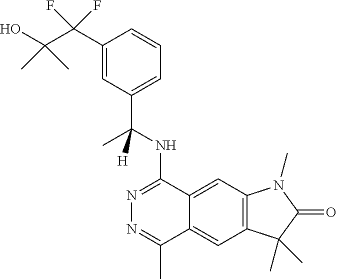 [SMILES: Cc1nnc(N[C@H](C)c2cccc(C(F)(F)C(C)(C)O)c2)c2cc3c(cc12)C(C)(C)C(=O)N3C] | UPLC-MS (ES⁺, Long acidic): 2.96 min, m/z 469.3 [M + H]⁺ (100%) ¹H-NMR (400 MHz, DMSO-d₆): 7.99 (s, 1H), 7.96 (s, 1H), 7.54-7.50 (m, 2H), 7.40-7.32 (m, 2H), 7.31-7.28 (m, 1H), 5.58-5.50 (m, 1H), 5.21 (s, 1H), 2.64 (s, 3H), 1.61 (d, J = 7.1 Hz, 3H), 1.39 (s, 3H), 1.39 (s, 3H), 1.12 (s, 3H), 1.09 (s, 3H)- CH₃ under solvent peak |
| 78 | 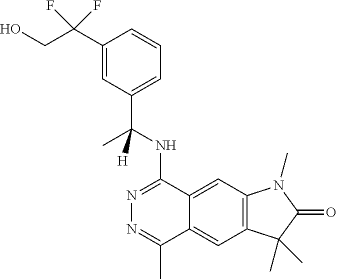 [SMILES: Cc1nnc(N[C@H](C)c2cccc(C(F)(F)CO)c2)c2cc3c(cc12)C(C)(C)C(=O)N3C] | UPLC-MS (ES⁺, Long acidic): 2.73 min, m/z 441.3 [M + H]⁺ (100%) ¹H-NMR (400 MHz, DMSO-d₆): 7.99 (s, 1H), 7.96 (s, 1H), 7.59 (br s, 1H), 7.57-7.53 (m, 1H), 7.43-7.32 (m, 3H), 5.62-5.51 (m, 2H), 3.87-3.76 (m, 2H), 2.64 (s, 3H), 1.61 (d, J = 7.0 Hz, 3H), 1.39 (s, 3H), 1.39 (s, 3H). CH₃ under solvent peak |
| 79 | 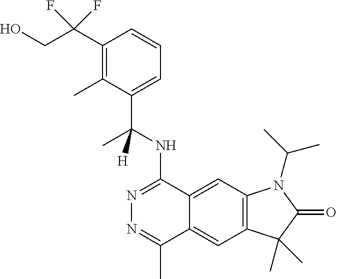 [SMILES: Cc1c([C@@H](C)Nc2nnc(C)c3cc4c(cc23)N(C(C)C)C(=O)C4(C)C)cccc1C(F)(F)CO] | UPLC-MS (ES⁺, Long acidic): 3.15 min, m/z 483.3 [M + H]⁺ (100%) ¹H-NMR (400 MHz, DMSO-d₆): 7.98- 7.96 (m, 2H), 7.60 (d, J = 7.6 Hz, 1H), 7.46-7.42 (m, 1H), 7.34-7.30 (m, 1H), 7.23-7.19 (m, 1H), 5.77-5.70 (m, 1H), 5.67 (t, J = 6.4 Hz, 1H), 4.67-4.57 (m, 1H), 3.87-3.85 (m, 2H), 2.61 (s, 3H), 2.54 (s, 3H), 1.58-1.53 (m, 9H), 1.36 (s, 3H), 1.35 (s, 3H). |
| 80 | 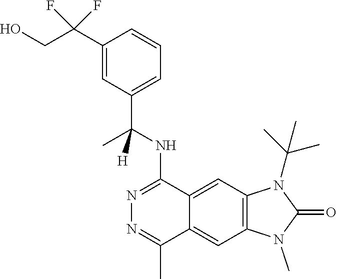 [SMILES: Cc1nnc(N[C@H](C)c2cccc(C(F)(F)CO)c2)c2cc3c(cc12)n(C)c(=O)n3C(C)(C)C] | UPLC-MS (ES⁺, Long acidic): 2.99 min, m/z 470.3 [M + H]⁺ (100%) 1H NMR (400 MHz, DMSO-d₆,): 8.15 (s, 1H), 7.62 (br s, 1H), 7.56 (br d, J = 7.8 Hz, 1H), 7.53 (s, 1H), 7.47 (d, J = 7.5 Hz, 1H), 7.43-7.37 (m, 1H), 7.35- 7.31 (m, 1H), 5.66-5.56 (m, 2H), 3.87- 3.76 (m, 2H), 3.41 (s, 3H), 2.65 (s, 3H), 1.85 (s, 9H), 1.62 (d, J = 7.2 Hz, 3H). |
| 81 | 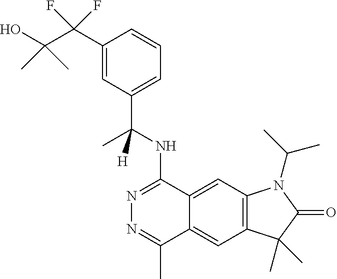 [SMILES: Cc1nnc(N[C@H](C)c2cccc(C(F)(F)C(C)(C)O)c2)c2cc3c(cc12)C(C)(C)C(=O)N3C(C)C] | UPLC-MS (ES⁺, Long acidic, 3.24 min, m/z 497.4 [M + H]⁺ (100%) ¹H-NMR (400 MHz, DMSO-d₆): 7.97 (s, 1H), 7.94 (s, 1H), 7.56-7.50 (m, 2H), 7.41-7.34 (m, 2H), 7.31-7.28 (m, 1H), 5.63-5.54 (m, 1H), 5.21 (s, 1H), 4.62 (sept, J = 6.9 Hz, 1H), 2.63 (s, 3H), 1.63 (d, J = 7.0 Hz, 3H), 1.57-1.52 (m, 6H), 1.36 (s, 3H), 1.36 (s, 3H), 1.12 (s, 3H), 1.08 (s, 3H). |
| 82 | 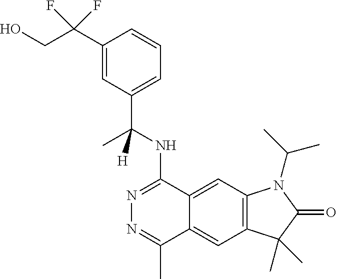 [SMILES: Cc1nnc(N[C@H](C)c2cccc(C(F)(F)CO)c2)c2cc3c(cc12)C(C)(C)C(=O)N3C(C)C] | UPLC-MS (ES⁺, Long acidic): 3.07 min, m/z 469.3 [M + H]⁺ (100%) ¹H-NMR (400 MHz, DMSO-d₆): 7.99 (s, 1H), 7.96 (s, 1H), 7.60 (br s, 1H), 7.55 (d, J = 7.5 Hz, 1H), 7.46 (br s, 1H), 7.43-7.38 (m, 1H), 7.36-7.32 (m, 1H), 5.62-5.55 (m, 2H), 4.67-4.58 (m, 1H), 3.87-3.77 (m, 2H), 2.63 (s, 3H), 1.63 (d, J = 7.1 Hz, 3H), 1.57-1.53 (m, 6H), 1.37 (s, 3H), 1.36 (s, 3H). |
| 83 | 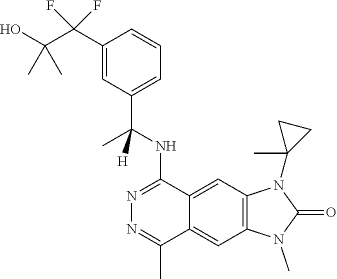 [SMILES: Cc1nnc(N[C@H](C)c2cccc(C(F)(F)C(C)(C)O)c2)c2cc3c(cc12)n(C)c(=O)n3C1(C)CC1] | UPLC-MS (ES⁺, Long acidic): 3.04 min, m/z 496.3 [M + H]⁺ (100%) ¹H-NMR (400 MHz, DMSO-d₆): 8.12 (s, 1H), 7.59-7.52 (m, 3H), 7.45 (d, J = 7.7 Hz, 1H), 7.43-7.38 (m, 1H), 7.31- 7.27 (m, 1H), 5.67-5.58 (m, 1H), 5.19 (s, 1H), 3.43 (s, 3H), 2.65 (s, 3H), 1.66 (d, J = 7.1 Hz, 3H), 1.52 (s, 3H), 1.13- 1.06 (m, 10H). |
| 84 | 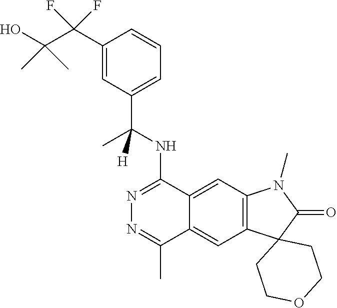 [SMILES: Cc1nnc(N[C@H](C)c2cccc(C(F)(F)C(C)(C)O)c2)c2cc3c(cc12)C1(CCOCC1)C(=O)N3C] | UPLC-MS (ES⁺, Long acidic): 2.93 min, m/z 511.3 [M + H]⁺ (100%) ¹H-NMR (400 MHz, DMSO-d₆): 8.04 (s, 1H), 7.95 (s, 1H), 7.54-7.49 (m, 2H), 7.38-7.32 (m, 2H), 7.31-7.27 (m, 1H), 5.58-5.50 (m, 1H), 5.20 (s, 1H), 4.16-4.08 (m, 2H), 3.91-3.84 (m, 2H), 3.31 (s, 3H), 2.65 (s, 3H), 2.06-1.96 (m, 2H), 1.79-1.72 (m, 2H), 1.61 (d, J = 7.0 Hz, 3H), 1.12 (s, 3H), 1.09 (s, 3H). |
| 85 | 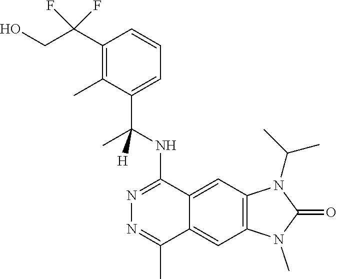 [SMILES: Cc1c([C@@H](C)Nc2nnc(C)c3cc4c(cc23)n(C(C)C)c(=O)n4C)cccc1C(F)(F)CO] | UPLC-MS (ES⁺, Long acidic): 2.94 min, m/z 470.3 [M + H]⁺ (100%) ¹H-NMR (400 MHz, DMSO-d₆): 8.20 (s, 1H), 7.62 (d, J = 7.6 Hz, 1H), 7.57 (s, 1H), 7.38 (d, J = 7.2 Hz, 1H), 7.33-7.29 (m, 1H), 7.23-7.17 (m, 1H), 5.79-5.71 (m, 1H), 5.67 (t, J = 6.4 Hz, 1H), 4.78- 4.70 (m, 1H), 3.96-3.85 (m, 2H), 3.45 (s, 3H), 2.64 (s, 3H), 2.55 (s, 3H), 1.62- 1.58 (m, 6H), 1.56 (d, J = 6.9 Hz, 3H). |
| 86 | 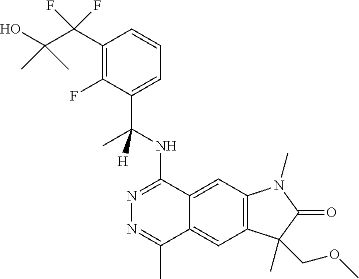 [SMILES: COCC1(C)C(=O)N(C)c2cc3c(N[C@H](C)c4cccc(C(F)(F)C(C)(C)O)c4F)nnc(C)c3cc21] | UPLC-MS (ES⁺, Long acidic): 3.00 min, m/z 517.3 [M + H]⁺ (100%) 1H NMR (400 MHz, DMSO-d₆) 8.00 (s, 1H), 7.98-7.96 (m, 1H), 7.54-7.5 (m, 1H), 7.41-7.38 (m, 1H), 7.29-7.26 (m, 1H), 7.17-7.12 (m, 1H), 5.76-5.70 (m, 1H), 5.34 (s, 1H), 3.85-3.83 (m, 1H), 3.68-3.65 (m, 1H), 3.32 (s, 3H), 3.07- 3.06 (m, 3H), 2.63-2.62 (m, 3H), 1.60 (d, J = 7.5 Hz, 3H), 1.31-1.30 (m, 3H), 1.24 (s, 3H), 1.23 (s, 3H). |
| 87 | 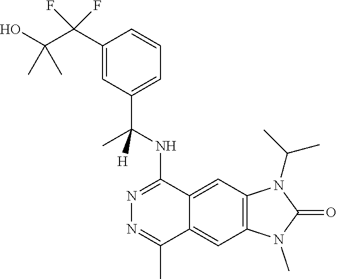 [SMILES: Cc1nnc(N[C@H](C)c2cccc(C(F)(F)C(C)(C)O)c2)c2cc3c(cc12)n(C)c(=O)n3C(C)C] | UPLC-MS (ES⁺, Long acidic): 3.07 min, m/z 484.2 [M + H]⁺ (100%) ¹H-NMR (400 MHz, DMSO-d₆): 8.17 (s, 1H), 7.58 (s, 1H), 7.56-7.51 (m, 2H), 7.39-7.33 (m, 2H), 7.31-7.27 (m, 1H), 5.65-5.56 (m, 1H), 5.20 (s, 1H), 4.74 (sept, J = 7.0 Hz, 1H), 3.45 (s, 3H), 2.65 (s, 3H), 1.63 (d, J = 6.9 Hz, 3H), 1.61-1.56 (m, 6H), 1.11 (s, 3H), 1.06 (s, 3H). |
| 88 | 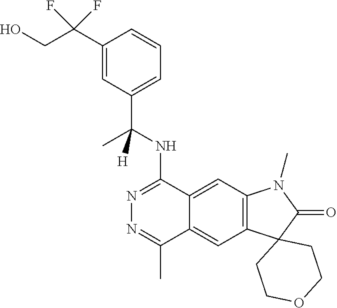 [SMILES: Cc1nnc(N[C@H](C)c2cccc(C(F)(F)CO)c2)c2cc3c(cc12)C1(CCOCC1)C(=O)N3C] | UPLC-MS (ES⁺, Long acidic): 2.73 min, m/z 483.2 [M + H]⁺ (100%). ¹H-NMR (400 MHz, DMSO-d₆): 8.04 (s, 1H), 7.95 (s, 1H), 7.59 (br s, 1H), 7.55 (d, J = 7.1 Hz, 1H), 7.42-7.32 (m, 3H), 5.62-5.53 (m, 2H), 4.16-4.08 (m, 2H), 3.91-3.76 (m, 4H), 3.31 (s, 3H,), 2.66 (s, 3H), 2.06-1.97 (m, 2H), 1.80- 1.73 (m, 2H), 1.61 (d, J = 7.1 Hz, 3H). |
| 89 | 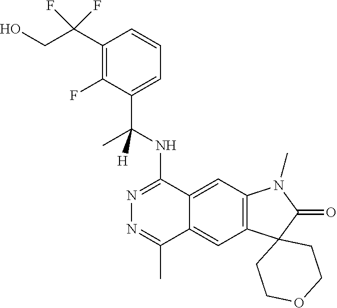 [SMILES: Cc1nnc(N[C@H](C)c2cccc(C(F)(F)CO)c2F)c2cc3c(cc12)C1(CCOCC1)C(=O)N3C] | UPLC-MS (ES⁺, Long acidic): 2.78 min, m/z 501.2 [M + H]⁺ ¹H-NMR (400 MHz, DMSO-d₆): 8.06 (s, 1H), 7.99 (s, 1H), 7.57-7.51 (m, 1H), 7.43-7.53 (m, 2H), 7.21-7.15 (m, 1H), 5.77-5.70 (m, 2H), 4.17-4.08 (m, 2H), 4.00-3.83 (m, 4H), 3.33 (s, 3H), 2.65 (s, 3H), 2.07-1.96 (m, 2H), 1.80- 1,73 (m, 2H), 1.61 (d, J = 7.0 Hz, 3H). |
| 90 | 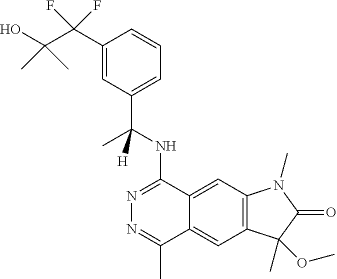 [SMILES: COC1(C)C(=O)N(C)c2cc3c(N[C@H](C)c4cccc(C(F)(F)C(C)(C)O)c4)nnc(C)c3cc21] | UPLC-MS (ES⁺, Long acidic): 2.90 + 2.91 min, m/z 485.3 [M + H]⁺ (100%) 1H NMR (400 MHz, DMSO-d₆): 8.00 (s, 1H), 7.96 (s, 1H), 7.56-7.49 (m, 2H), 7.44-7.32 (m, 2H), 7.33-7.27 (m, 1H), 5.55 (quint, J = 6.7 Hz, 1H), 5.21 (s, 1H), 3.33 (s, 3H), 2.91 (d, J = 2.9 Hz, 3H), 2.65 (s, 3H), 1.62 (d, J = 6.3 Hz, 3H), 1.54 (s, 3H), 1.16-1.06 (m, 6H) |
| 91 | 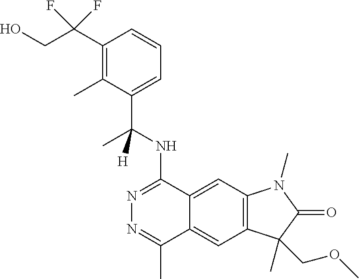 | UPLC-MS (ES⁺, Long acidic): 3.00 min, m/z 499.3 [M + H]⁺ (95%) 1H NMR (400 MHz, DMSO-d₆): 8.00 (s, 1H), 7.94 (s, 1H), 7.54-7.51 (m, 1H), 7.40-7.37 (m, 2H), 7.30-7.27 (m, 1H), 5.56-5.50 (m, 1H), 5.21-5.20 (m, 1H), 3,83 (d, J = 9.2 HZ, 1H), 3.66 (d, J = 9.1 Hz, 1H), 3.23 (s, 3H), 3.06 (d, J = 2.8 Hz, 3H), 2.63 (s, 3H), 1.62 (d, J = 6.4 Hz, 3H), 1,30 (d, J = 2.9 Hz, 3H) 1.12 (s, 3H), 1,09 (s, 3H). |
| 92 | 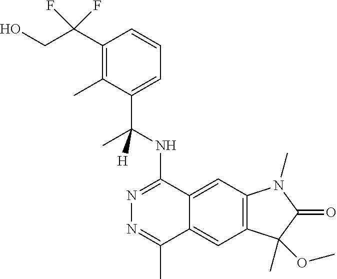 [SMILES: COC1(C)C(=O)N(C)c2cc3c(N[C@H](C)c4cccc(C(F)(F)CO)c4C)nnc(C)c3cc21] | UPLC-MS (ES⁺, Long acidic): 2.78 min, m/z 471.6 [M + H]⁺ (98%) 1H NMR (400 MHz, DMSO-d₆): 8.03 (s, 1H), 7.95 (s, 1H), 7.65-7.57 (m, 1H), 7.49-7.41 (m, 1H), 7.35-7.29 (m, 1H), 7.25-7.16 (m, 1H), 5.78-5.69 (m, 1H), 5.67 (t, J = 6.4 Hz, 1H), 4.00-3.80 (m, 2H), 3.33 (d, J = 0.96 Hz, 3H), 2.91 (d, J = 6.4 Hz, 3H), 2.64 (s, 3H), 2.53 (s, 3H), 1.59-1.50 (m, 6H) |
| 93 | 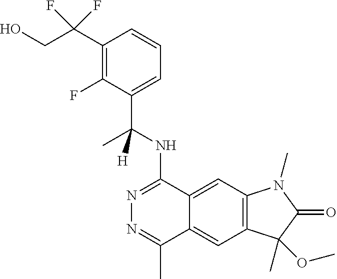 [SMILES: COC1(C)C(=O)N(C)c2cc3c(N[C@H](C)c4cccc(C(F)(F)CO)c4F)nnc(C)c3cc21] | UPLC-MS (ES⁺, Long acidic): 2.74 min, m/z 475.5 [M + H]⁺ (100%) 1H NMR (400 MHz, DMSO-d₆) 8.04 (s, 1H), 7.97 (s, 1H), 7.62-7.52 (m, 1H), 7.51-7.43 (m, 1H), 7.42-7.34 (m, 1H), 7.25-7.14 (m, 1H), 5.79-5.68 (m, 2H), 4.04-3.87 (m, 2H), 3.35 (s, 3H), 2.92 (d, J = 2.9 Hz, 3H), 2.65 (s, 3H), 1.62 (d, J = 7.0 Hz, 3H), 1.54 (d, J = 1.3 Hz, 3H) |
| 94 | 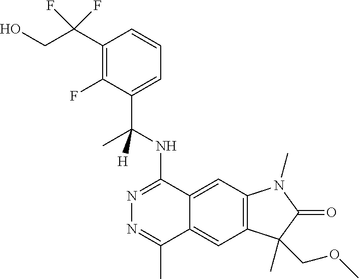 [SMILES: COCC1(C)C(=O)N(C)c2cc3c(N[C@H](C)c4cccc(C(F)(F)CO)c4F)nnc(C)c3cc21] | UPLC-MS (ES⁺, Long acidic): 2.80 min, m/z 489.8 [M + H]⁺ (100%) 1H NMR (400 MHz, DMSO-d₆): 8.00 (s, 1H), 7.96 (d, J = 2.3 Hz, 1H), 7.58-7.53 (m, 1H), 7.41-7.36 (m, 2H), 7.21-7.16 (m, 1H), 5.76-5.73 (m, 2H), 3.99-3.92 (m, 2H), 3.85 (d, J = 8.6 Hz, 1H) 3.65 (dd, J = 8.6 and 1.9 Hz, 1H), 3.32 (s, 3H), 3.07 (d, J = 2.0 Hz, 3H), 2.63 (d, J = 1.2 Hz, 3H), 1.61 (d, J = 6.2 Hz, 3H), 1.30 (d, J = 1.9 Hz, 3H) |
| 95 | 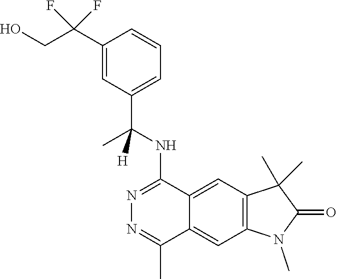 [SMILES: Cc1nnc(N[C@H](C)c2cccc(C(F)(F)CO)c2)c2cc3c(cc12)N(C)C(=O)C3(C)C] | UPLC-MS (ES⁺, Long acidic): 2.78 min, m/z 441.5 [M + H]⁺ (100%) ¹H-NMR (400 MHz, DMSO-d₆): 8.47 (s, 1H), 7.59 (br s, 1H), 7.57-7.53 (m, 1H), 7.43-7.37 (m, 1H), 7.36-7.30 (m, 3H), 5.60 (t, J = 6.4 Hz, 1H), 5.56-5.50 (m, 1H), 3.87-3.77 (m, 2H), 3.29 (s, 3H), 2.65 (s, 3H), 1.60 (d, J = 7.0 Hz, 3H), 1.42 (s, 3H), 1.41 (s, 3H). |
| 96 | 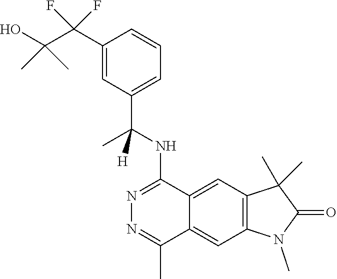 [SMILES: Cc1nnc(N[C@H](C)c2cccc(C(F)(F)C(C)(C)O)c2)c2cc3c(cc12)N(C)C(=O)C3(C)C] | UPLC-MS (ES⁺, Long acidic): 3.03 min, m/z 469.6 [M + H]⁺ (100%) ¹H-NMR (400 MHz, DMSO-d₆): 8.47 (s, 1H), 7.55-7.50 (m, 2H), 7.38-7.33 (m, 2H), 7.32-7.27 (m, 2H), 5.58-5.50 (m, 1H), 5.20 (s, 1H), 3.28 (s, 3H), 2.65 (s, 3H), 1.60 (d, J = 7.0 Hz, 3H), 1.40 (s, 6H), 1.16 (s, 3H), 1.07 (s, 3H). |
| 97 | 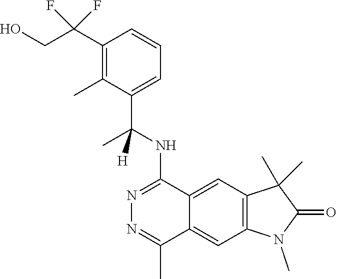 [SMILES: Cc1c([C@@H](C)Nc2nnc(C)c3cc4c(cc23)C(C)(C)C(=O)N4C)cccc1C(F)(F)CO] | UPLC-MS (ES⁺, Long acidic): 2.89 min, m/z 455.7 [M + H]⁺ (100%) ¹H-NMR (400 MHz, DMSO-d₆): 8.50 (s, 1H), 7.61 (d, J = 7.4 Hz, 1H), 7.37- 7.30 (m, 3H), 7.24-7.18 (m, 1H), 5.74- 5.68 (m, 1H), 5.66 (t, J = 6.4 Hz, 1H), 3.97-3.85 (m, 2H), 3.28 (s, 3H), 2.64 (s, 3H), 2.54 (s, 3H), 1.53 (d, J = 6.9 Hz, 3H), 1.42 (s, 3H), 1.41 (s, 3H). |
| 98 | 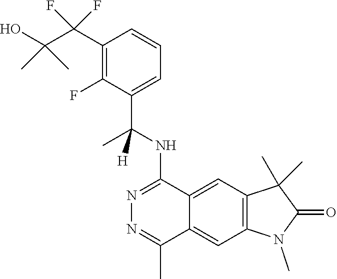 [SMILES: Cc1nnc(N[C@H](C)c2cccc(C(F)(F)C(C)(C)O)c2F)c2cc3c(cc12)N(C)C(=O)C3(C)C] | UPLC-MS (ES⁺, Long acidic): 3.07 min, m/z 487.7 [M + H]⁺ (100%) ¹H-NMR (400 MHz, DMSO-d₆): 8.51 (s, 1H), 7.55-7.50 (m, 1H), 7.38-7.33 (m, 2H), 7.30-7.25 (m, 1H), 7.19-7.13 (m, 1H), 5.77-5.66 (m, 1H), 5.34 (s, 1H), 3.29 (s, 3H), 2.64 (s, 3H), 1.59 (d, J = 7.0 Hz, 3H), 1.43 (s, 3H), 1.42 (s, 3H), 1.24 (s, 3H), 1.22 (s, 3H). |
| 99 | 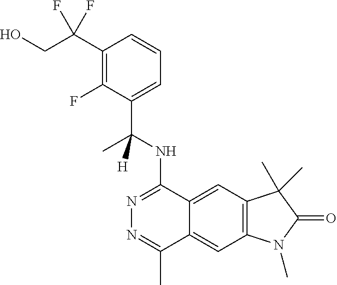 [SMILES: Cc1nnc(N[C@H](C)c2cccc(C(F)(F)CO)c2F)c2cc3c(cc12)N(C)C(=O)C3(C)C] | UPLC-MS (ES⁺, Long acidic): 2.84 min, m/z 459.6 [M + H]⁺ (100%) ¹H-NMR (400 MHz, DMSO-d₆): 8.51 (s, 1H), 7.58-7.53 (m, 1H), 7.41-7.34 (m, 3H), 7.23-7.17 (m, 1H), 5.77-5.68 (m, 2H), 4.01-3.90 (m, 2H), 3.29 (s, 3H), 2.64 (s, 3H), 1.60 (d, J = 7.1 Hz, 3H), 1.43 (s, 3H), 1.42 (s, 3H). |
| 100 | 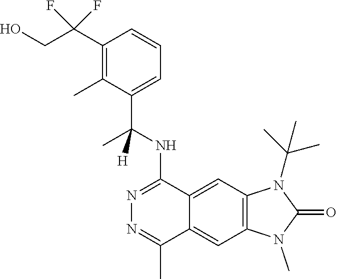 [SMILES: Cc1c([C@@H](C)Nc2nnc(C)c3cc4c(cc23)n(C(C)(C)C)c(=O)n4C)cccc1C(F)(F)CO] | UPLC-MS (ES⁺, Long acidic): 3.17, m/z 484.6 [M + H]⁺ (100%) 1H NMR (400 MHz, DMSO-d₆): 8.17 (s, 1H), 7.64 (d, J = 8.1 Hz, 1H), 7.51 (s, 1H), 7.49 (d, J = 6.7 Hz, 1H), 7.32- 7.29 (m, 1H), 7.22-7.16 (m, 1H), 5.78- 5.64 (m, 2H), 3.97-3.86 (m, 2H), 3.41 (s, 3H), 2.63 (s, 3H), 2.55 (s, 3H), 1.86 (s, 9H), 1.55 (d, J = 6.9 Hz, 3H) |
| 101 | 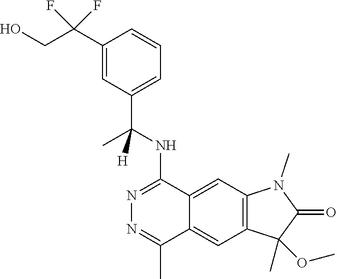 [SMILES: COC1(C)C(=O)N(C)c2cc3c(N[C@H](C)c4cccc(C(F)(F)CO)c4)nnc(C)c3cc21] | UPLC-MS (ES⁺, Long acidic): 2.66 min, m/z 457.7 [M + H]⁺ (100%) 1H NMR (400 MHz, DMSO-d₆): 8.00 (s, 1H), 7.96 (s, 1H), 7.63-7.58 (m, 1H), 7.58-7.52 (m, 1H), 7.48-7.37 (m, 2H), 7.37-7.31 (m, 1H), 5.65-5.50 (m, 2H), 3.90-3.72 (m, 2H), 3.33 (s, 3H), 2.92 (d, J = 2.7 Hz, 3H), 2.65 (s, 3H), 1.61 (d, J = 6.2 Hz, 3H), 1.54 (s, 3H) |
| 102 | 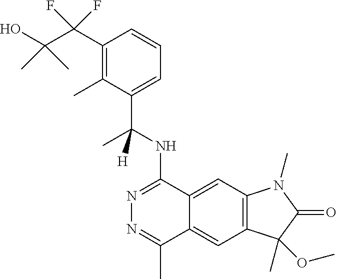 [SMILES: COC1(C)C(=O)N(C)c2cc3c(N[C@H](C)c4cccc(C(F)(F)C(C)(C)O)c4C)nnc(C)c3cc21] | UPLC-MS (ES⁺, Long acidic): 2.99 min, m/z 499.7 [M + H]⁺ (100%) 1H NMR (400 MHz, DMSO-d₆): 8.03 (s, 1H), 7.94 (s, 1H), 7.64-7.55 (m, 1H), 7.45-7.37 (m, 1H), 7.27-7.21 (m, 1H), 7.21-7.12 (m, 1H), 5.85-5.70 (m, 1H), 5.30 (s, 1H), 3.33 (s, 3H), 2.91 (d, J = 5.8 Hz, 3H), 2.64 (s, 3H), 2.58 (s, 3H), 1.58-1.49 (m, 6H), 1.24 (s, 3H), 1.23 (s, 3H) |
| 103 | 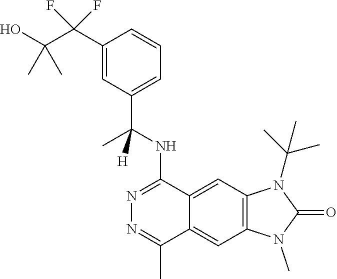 | UPLC-MS (ES⁺, Long acidic): 3.26, m/z 456.4 [M + H]⁺ (100%) 1H NMR (400 MHz, DMSO-d₆): 8.15 (s, 1H), 7.58-7.50 (m, 3H), 7.50-7.45 (m, 1H), 7.39-7.34 (m, 1H), 7.31-7.27 (m, 1H), 5.66-5.57 (m, 1H), 5.20 (s, 1H), 3.41 (s, 3H), 2.65 (s, 3H), 1.84 (s, 9H), 1.63 (d, J = 7.0 Hz, 3H), 1.12 (s, 3H), 1.07 (s, 3H) |
| 104 | 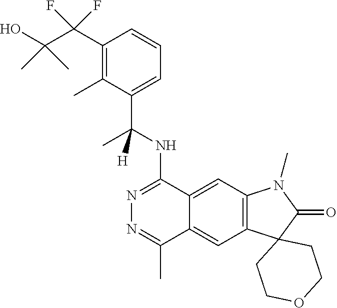 [SMILES: Cc1c([C@@H](C)Nc2nnc(C)c3cc4c(cc23)N(C)C(=O)C42CCOCC2)cccc1C(F)(F)C(C)(C)O] | UPLC-MS (ES⁺, Long acidic): 3.22 min, m/z 525.1 [M + H]⁺ (100%) ¹H-NMR (400 MHz, DMSO-d₆) δ 8.02 (s, 1H), 7.99 (s, 1H), 7.61-7.56 (m, 1H), 7.39-7.34 (m, 1H), 7.25-7.13 (m, 2H), 5.81-5.73 (m, 1H), 5.31 (s, 1H), 4.16-4.07 (m, 2H), 3.91-3.83 (m, 2H), 2.69-2.62 (m, 6H), 2.06-1.96 (m, 2H), 1.80-1.71 (m, 2H), 1.55 (d, J = 7.3 Hz, 3H), 1.28-1.21 (m, 6H). CH₃ under solvent peak. |
| 105 | 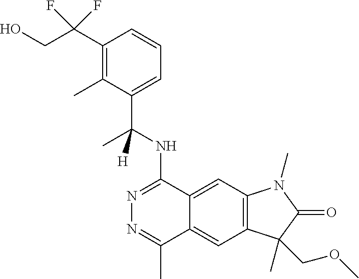 [SMILES: COCC1(C)C(=O)N(C)c2cc3c(N[C@H](C)c4cccc(C(F)(F)CO)c4C)nnc(C)c3cc21] | UPLC-MS (ES⁺, Long acidic): 3.07 min, m/z 485.7 [M + H]⁺ (100%) 1H NMR (400 MHz, DMSO-d₆): 7.97 (s, 1H), 7.96 (s, 1H), 7.62-7.60 (m, 1H), 7.39-7.36 (m, 1H), 7.32-7.30 (m, 1H), 7.22-7.18 (m, 1H), 5.75- 5.69 (m, 1H), 5.69-5.65 (m, 1H), 3.96- 3.87 (m, 2H), 3.84-3.82 (m, 1H), 3,67- 3.653 (m, 1H), 3.31 (s, 3H), 3.06- 3.05 (m, 3H), 2.63 (s, 3H), 2.54 (s, 3H), 1.55 (d, J = 6.7 Hz, 3H), 1.30 (d, J = 5.0 Hz, 3H). |
| 106 | 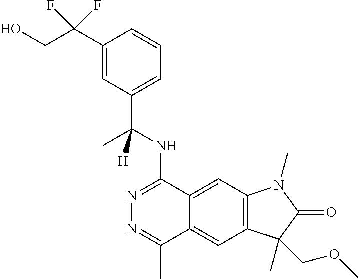 [SMILES: COCC1(C)C(=O)N(C)c2cc3c(N[C@H](C)c4cccc(C(F)(F)CO)c4)nnc(C)c3cc21] | UPLC-MS (ES⁺, Long acidic): 2.95 min, m/z 471.7 [M + H]⁺ (100%) 1H NMR (400 MHz, DMSO- d₆): 7.98 (s, 1H), 7.92 (s, 1H), 7.59 (s, 1H), 7.57-7.54 (m, 1H), 7.43-7.36 (m, 1H), 7.35-7.31 (m, 2H), 5.59-5.51 (m, 2H), 3.84-3.82 (m, 2H), 3.79-3.76 (m, 1H), 3.67-3.64 (m, 1H), 3.31 (s, 3H), 3.07 (s, 3H), 2.63 (s, 3H), 1.62-1.60 (m, 3H), 1.31 (s, 3H). |
| 107 | 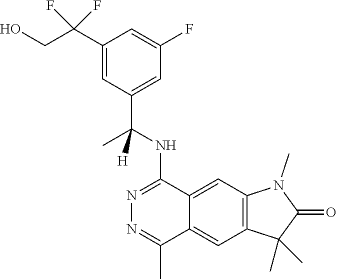 [SMILES: Cc1nnc(N[C@H](C)c2cc(F)cc(C(F)(F)CO)c2)c2cc3c(cc12)C(C)(C)C(=O)N3C] | UPLC-MS (ES⁺, Long acidic): 3.12 min, m/z 459.6 [M + H]⁺ (100%) 1H NMR (400 MHz, DMSO-d₆): 8.00 (s, 1H), 7.93 (s, 1H), 7.46 (s, 1H), 7.42- 7.33 (m, 2H), 7.22-7.13 (m, 1H), 5.65 (t, J = 6.3 Hz, 1H), 5.53 (quintet, J = 7.2 Hz, 1H), 3.92-3.73 (m, 2H), 3.32 (s, 3H), 2.64 (s, 3H), 1.61 (d, J = 7.0 Hz, 3H), 1.39 (s, 6H) |
| 108 | 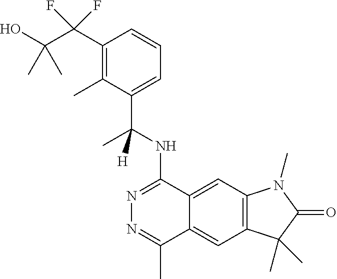 [SMILES: Cc1c([C@@H](C)Nc2nnc(C)c3cc4c(cc23)N(C)C(=O)C4(C)C)cccc1C(F)(F)C(C)(C)O] | UPLC-MS (ES⁺, Long acidic): 3.09 min, m/z 483.6 [M + H]⁺ (87%) 1H NMR (400 MHz, DMSO-d₆): 7.97 (d, J = 6.5 Hz, 2H), 7.59 (d, J = 8.3 Hz, 1H), 7.35 (d, J = 7.4 Hz, 1H), 7.23 (d, J = 6.6 Hz, 1H), 7.18-7.14 (m, 1H), 5.79-5.76 (m, 1H), 5.30 (s, 1H), 2.63 (s, 3H), 2.58 (s, 3H), 1.55 (d, J = 6.7 Hz, 3H), 1.39 (d, J = 4.0 Hz, 6H), 1.23 (d, J = 6.2 Hz, 6H). CH₃ under solvent peak |
| 109 | 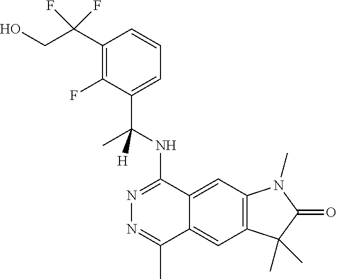 [SMILES: Cc1nnc(N[C@H](C)c2cccc(C(F)(F)CO)c2F)c2cc3c(cc12)C(C)(C)C(=O)N3C] | UPLC-MS (ES⁺, Long acidic): 2.77 min, m/z 459.4 [M + H]⁺ (100%) 1H NMR (400 MHz, MeOD): 8.00 (s, 1H), 7.98 (s, 1H), 7.60-7.54 (m, 1H), 7.47-7.40 (m, 1H), 7.18-7.12 (m, 1H), 5.80-5.72 (m, 1H), 4.11-3.99 (m, 2H), 3.44 (s, 3H), 2.72 (s, 3H), 1.73 (d, J = 7.8 Hz, 3H), 1.53-1.46 (m, 6H) |
| 110 | 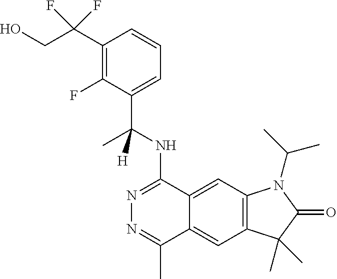 [SMILES: Cc1nnc(N[C@H](C)c2cccc(C(F)(F)CO)c2F)c2cc3c(cc12)C(C)(C)C(=O)N3C(C)C] | UPLC-MS (ES⁺, Long acidic): 3.08 min, m/z 487.6 [M + H]⁺ (100%) ¹H-NMR (400 MHz, DMSO-d₆): 8.02- 7.97 (m, 2H), 7.58-7.52 (m, 1H), 7.49- 7.44 (m, 1H), 7.42-7.36 (m, 1H), 7.22- 7.17 (m, 1H), 5.77-5.72 (m, 2H), 4.69- 4.59 (m, 1H), 4.03-3.89 (m, 2H), 2.62 (s, 3H), 1.63 (d, J = 7.0 Hz, 3H), 1.59- 1.54 (m, 6H), 1.39-1.34 (m, 6H). |
| 111 | 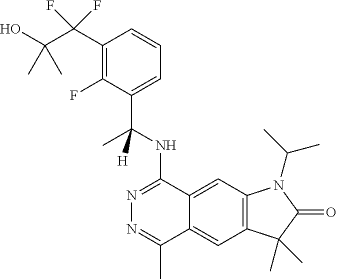 [SMILES: Cc1nnc(N[C@H](C)c2cccc(C(F)(F)C(C)(C)O)c2F)c2cc3c(cc12)C(C)(C)C(=O)N3C(C)C] | UPLC-MS (ES⁺, Long acidic): 3.24 min, m/z 515.7 [M + H]⁺ (100%) ¹H-NMR (400 MHz, DMSO-d₆): 8.00- 7.97 (m, 2H), 7.55-7.48 (m, 1H), 7.45- 7.40 (m, 1H), 7.31-7.26 (m, 1H), 7.18- 7.13 (m, 1H), 5.79-5.72 (m, 1H), 5.35 (s, 1H), 4.67-4.58 (m, 1H), 2.62 (s, 3H), 1.61 (d, J = 7.0 Hz, 3H), 1.59-1.54 (m, 6H), 1.39-1.35 (m, 6H), 1.26-1.21 (m, 6H). |
| 112 | 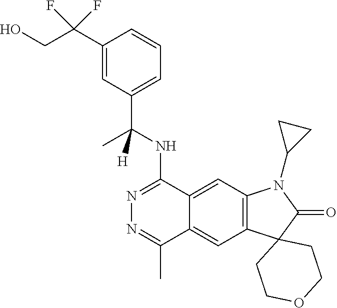 [SMILES: Cc1nnc(N[C@H](C)c2cccc(C(F)(F)CO)c2)c2cc3c(cc12)C1(CCOCC1)C(=O)N3C1CC1] | UPLC-MS (ES⁺, Long acidic): 2.81 min, m/z 509.6 [M + H]⁺ (100%) ¹H-NMR (400 MHz, DMSO-d₆): 8.01 (s, 1H), 7.98 (s, 1H), 7.61 (br s, 1H), 7.57-7.53 (m, 1H), 7.52-7.49 (s, 1H), 7.43-7.37 (m, 1H), 7.36-7.32 (m, 1H), 5.62-5.54 (m, 2H), 4.13-4.05 (m, 2H), 3.89-3.77 (m, 4H), 2.89-2.80 (m, 1H), 2.65 (s, 3H), 2.03-1.93 (m, 2H), 1.78- 1.70 (m, 2H), 1.63 (d, J = 7.0 Hz, 3H), 1.18-1.12 (m, 2H), 0.98-0.85 (m, 2H). |
| 113 | 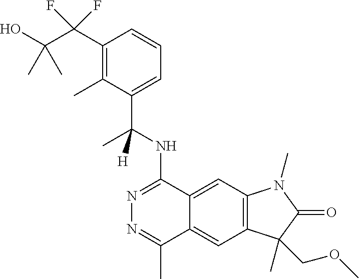 [SMILES: COCC1(C)C(=O)N(C)c2cc3c(N[C@H](C)c4cccc(C(F)(F)C(C)(C)O)c4C)nnc(C)c3cc21] | UPLC-MS (ES⁺, Long acidic): 3.11 min, m/z 513.6 [M + H]⁺ (98%) 1H NMR (400 MHz, DMSO-d₆) 7.97 (s, 1H), 7.96-7.94 (m, 1H), 7.61-7.58 (m, 1H), 7.36-7.32 (m, 1H), 7.25-7.22 (m, 1H), 7.18-7.14 (m, 1H), 5.79-5.73 (m, 1H), 5.30 (s, 1H), 3.84-3.81 (m, 1H), 3.67-3.64 (m, 1H), 3.06 (d, J = 5.0 Hz, 3H), 2.63 (s, 3H), 2.59 (s, 3H), 2.49 (s, 3H, under DMSO), 1.54 (d, J = 6.8 Hz, 3H), 1.30 (d, J = 4.5 Hz, 3H) 1.35 (s, 3H), 1.23 (s, 3H) |
| 114 | 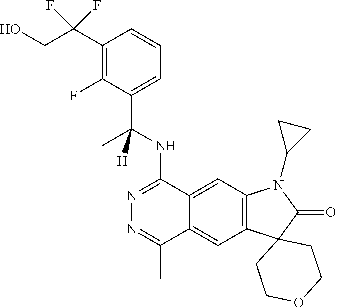 [SMILES: Cc1nnc(N[C@H](C)c2cccc(C(F)(F)CO)c2F)c2cc3c(cc12)C1(CCOCC1)C(=O)N3C1CC1] | UPLC-MS (ES⁺, Long acidic): 2.91 min, m/z 527.5 [M + H]⁺ (100%) ¹H-NMR (400 MHz, DMSO-d₆): 8.04- 8.00 (m, 2H), 7.58-7.51 (m, 2H), 7.41- 7,.35 (m, 1H), 7.21-7.15 (m, 1H), 5.78- 5.71 (m, 2H), 4.15-4.06 (m, 2H), 4.01- 3.91 (m, 2H), 3.89-3.82 (m, 2H), 2.88- 2.82 (m, 1H), 2.64 (s, 3H), 2.04-1.94 (m, 2H), 1.77-1.70 (m, 2H), 1.63 (d, J = 6.8 Hz, 3H), 1.21-1.12 (m, 2H), 1.02- 0.86 (m, 2H). |
| 115 | 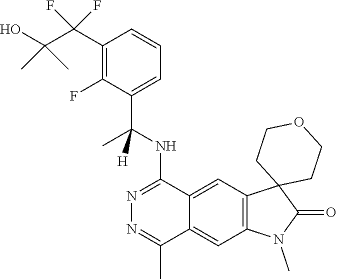 [SMILES: Cc1nnc(N[C@H](C)c2cccc(C(F)(F)C(C)(C)O)c2F)c2cc3c(cc12)N(C)C(=O)C31CCOCC1] | UPLC-MS (ES⁺, Long acidic): 2.93 min, m/z 529.5 [M + H]⁺ (100%) 1H NMR (400 MHz, DMSO-d₆) 8.63 (s, 1H), 7.55-7.50 (m, 1H), 7.48 (d, J = 7.1 Hz, 1H), 7.36 (s, 1H), 7.30-7.25 (m, 1H), 7.20-7.14 (m, 1H), 5.78- 5.71 (m, 1H), 5.34 (s, 1H), 4.13-4.08 (m, 2H), 4.03-3.97 (m, 2H), 3.28 (s, 3H), 2.64 (s, 3H), 2.00-1.91 (m, 2H), 1.86-1.80 (m, 2H), 1.60 (d, J = 6.9 Hz, 3H), 1.23 (d, J = 5.4 Hz, 6H) |
| 116 | 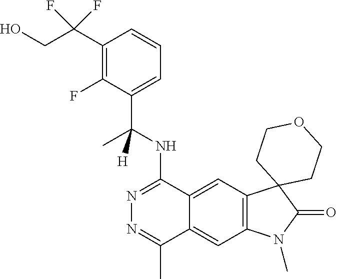 [SMILES: Cc1nnc(N[C@H](C)c2cccc(C(F)(F)CO)c2F)c2cc3c(cc12)N(C)C(=O)C31CCOCC1] | UPLC-MS (ES⁺, Long acidic): 2.73 min, m/z 501.4 [M + H]⁺ (100%) 1H NMR (400 MHz, DMSO-d₆) 8.63 (s, 1H), 7.59-7.53 (m, 1H), 7.50 (d, J = 7.9 Hz, 1H), 7.41-7.37 (m, 2H), 7.23-7.17 (m, 1H), 5.78-5.71 (m, 2H), 4.15-4.07 (m, 2H), 4.04-3.90 (m, 4H), 3.29 (s, 3H), 2.64 (s, 3H), 2.00- 1.90 (m, 2H), 1.89-1.82 (m, 2H), 1.61 (d, J = 7.0 Hz, 3H). |
| 117 | 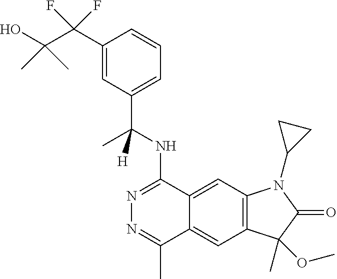 [SMILES: COC1(C)C(=O)N(C2CC2)c2cc3c(N[C@H](C)c4cccc(C(F)(F)C(C)(C)O)c4)nnc(C)c3cc21] | UPLC-MS (ES⁺, Long acidic): 3.00 + 3.01 min, m/z 511.6 [M + H]⁺ (40 + 57%), 1H NMR (400 MHz, DMSO-d₆): 8.03 (s, 1H), 7.94 (s, 1H), 7.60-7.48 (m, 3H), 7.41-7.33 (m, 1H), 7.33-7.26 (m, 1H), 5.65-5.53 (m, 1H), 5.21 (s, 1H), 2.92-2.83 (m, 4H), 2.65 (s, 3H), 1.63 (d, J = 7.0 Hz, 3H), 1.50 (s, 3H), 1.25- 1.14 (m, 2H), 1.14-1.05 (m, 6H), 0.99- 0.79 (m, 2H) |
| 118 | 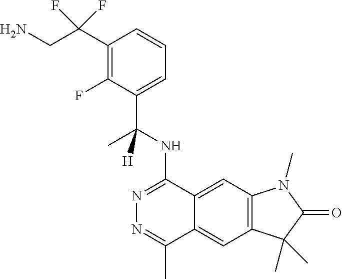 [SMILES: Cc1nnc(N[C@H](C)c2cccc(C(F)(F)CN)c2F)c2cc3c(cc12)C(C)(C)C(=O)N3C] | UPLC-MS (ES⁺, Long acidic): 2.15 min, m/z 458.3 [M + H]⁺ (100%) 1H NMR (400 MHz, DMSO-d₆) 7.99 (app s, 2H), 7.57-7.52 (m, 1H), 7.42- 7.35 (m, 2H), 7.21-7.16 (m, 1H), 5.77- 5.70 (m, 1H), 3.34 (s, 3H), 3.25 (t, J = 14.8 Hz, 2H), 2.63 (s, 3H), 1.62 (d, J = 6.9 Hz, 3H), 1.72 (br. s. 2H), 1.41- 1.37 (m, 6H) |
| 119 | 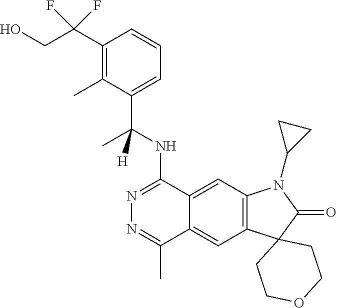 [SMILES: Cc1c([C@@H](C)Nc2nnc(C)c3cc4c(cc23)N(C2CC2)C(=O)C42CCOCC2)cccc1C(F)(F)CO] | UPLC-MS (ES⁺, Long acidic): 2.91 min, m/z 523.6 [M + H]⁺ (100%) ¹H-NMR (400 MHz, DMSO-d₆): 8.00 (app s, 2H), 7.64-7.61 (m, 1H), 7.53- 7.50 (m, 1H), 7.33-7.29 (m, 1H), 7.21- 7.16 (m, 1H), 5.75-5.70 (m, 1H), 5.70- 5.65 (m, 1H), 4.13-4.04 (m, 2H), 3.96- 3.81 (m, 4H), 2.88-2.81 (m, 1H), 2.63 (s, 3H), 2.04-1.93 (m, 2H), 1.78-1.70 (m, 2H), 1.56 (d, J = 7.0 Hz, 3H), 1.18- 1.14 (m, 2H), 0.98-0.94 (m, 1H), 0.91- 0.84 (m, 1H). CH₃ under solvent peak |
| 120 | 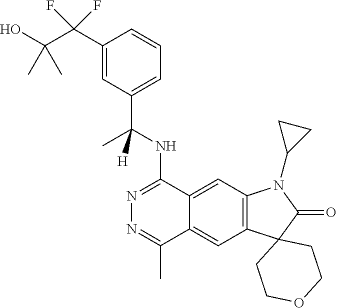 [SMILES: Cc1nnc(N[C@H](C)c2cccc(C(F)(F)C(C)(C)O)c2)c2cc3c(cc12)C1(CCOCC1)C(=O)N3C1CC1] | UPLC-MS (ES⁺, Long acidic): 3.02 min, m/z 537.5 [M + H]⁺ (100%) ¹H-NMR (400 MHz, DMSO-d₆): 8.01 (s, 1H), 7.97 (s, 1H), 7.56-7.46 (m, 3H), 7.39-7.33 (m, 1H), 7.32-7.27 (m, 1H), 5.62-5.52 (m, 1H), 5.20 (s, 1H), 4.14-4.04 (m, 2H), 3.89-3.81 (m, 2H), 2.86-2.80 (m, 1H), 2.65 (s, 3H), 2.03- 1.94 (m, 2H), 1.77-1.68 (m, 2H), 1.63 (d, J = 7.0 Hz, 3H), 1.17-1.07 (m, 8H), 0.96-0.85 (m, 2H). |
| 121 | 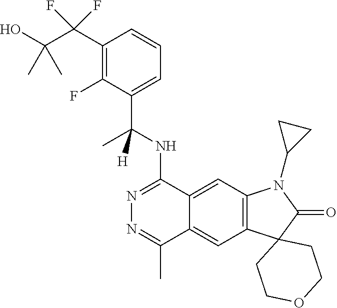 [SMILES: Cc1nnc(N[C@H](C)c2cccc(C(F)(F)C(C)(C)O)c2F)c2cc3c(cc12)C1(CCOCC1)C(=O)N3C1CC1] | UPLC-MS (ES⁺, Long acidic): 3.05 min, m/z 555.7 [M + H]⁺ (100%) ¹H-NMR (400 MHz, DMSO-d₆): 8.02 (s, 1H), 8.01 (s, 1H), 7.55-7.48 (m, 2H), 7.31-7.24 (m, 1H), 7.17-7.11 (m, 1H), 5.77-5.70 (m, 1H), 5.35 (s, 1H), 4.14-4.04 (m, 2H), 3.90-3.81 (m, 2H), 2.89-2.80 (m, 1H), 2.65 (s, 3H), 2.04- 1.95 (m, 2H), 1.78-1.70 (m, 2H), 1.61 (d, J = 7.0 Hz, 3H), 1.24 (s, 3H), 1.23 (s, 3H), 1.20-1.10 (m, 2H), 1.00-0.85 (m, 2H). |
| 122 | 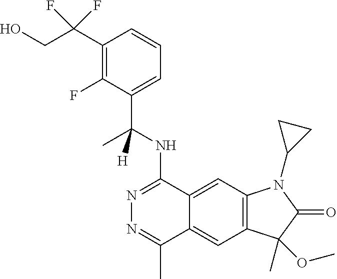 [SMILES: COC1(C)C(=O)N(C2CC2)c2cc3c(N[C@H](C)c4cccc(C(F)(F)CO)c4F)nnc(C)c3cc21] | UPLC-MS (ES⁺, Long acidic): 2.83 min, m/z 501.5 [M + H]⁺ (100%) 1H NMR (400 MHz, DMSO-d₆): 8.07 (s, 1H), 7.96 (s, 1H), 7.66-7.51 (m, 2H), 7.43-7.33 (m, 1H), 7.26-7.14 (m, 1H), 5.80-5.67 (m, 2H), 4.05-3.86 (m, 2H), 2.93-2.82 (m, 4H), 2.64 (s, 3H), 1.63 (d, J = 7.0 Hz, 3H), 1.50 (s, 3H), 1.27-1.09 (m, 2H), 1.06-0.79 (m, 2H) |
| 123 | 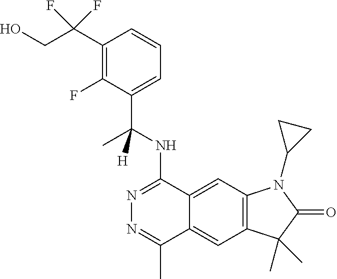 [SMILES: Cc1nnc(N[C@H](C)c2cccc(C(F)(F)CO)c2F)c2cc3c(cc12)C(C)(C)C(=O)N3C1CC1] | UPLC-MS (ES⁺, Long acidic): 2.90 min, m/z 485.7 [M + H]⁺ (100%) ¹H-NMR (400 MHz, DMSO-d₆): 8.02 (s, 1H), 7.97 (s, 1H), 7.59-7.50 (m, 2H), 7.40-7.35 (m, 1H), 7.22-7.15 (m, 1H), 5.78-5.70 (m, 2H), 4.02-3.89 (m, 2H), 2.91-2.84 (m, 1H), 2.62 (s, 3H), 1.63 (d, J = 6.9 Hz, 3H), 1.36 (s, 6H), 1.20-1.12 (m, 2H), 1.00-0.85 (m, 2H). |
| 124 | 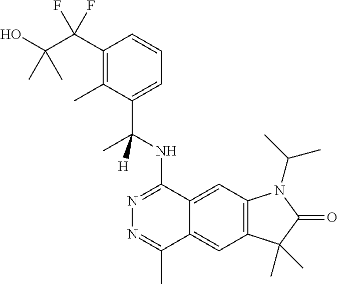 [SMILES: Cc1c([C@@H](C)Nc2nnc(C)c3cc4c(cc23)N(C(C)C)C(=O)C4(C)C)cccc1C(F)(F)C(C)(C)O] | UPLC-MS (ES⁺, Long acidic): 3.36 min, m/z 511.8 [M + H]⁺ (100%) ¹H-NMR (400 MHz, DMSO-d₆): 7.97 (app s, 2H), 7.61-7.57 (m, 1H), 7.43- 7.39 (m, 1H), 7.26-7.22 (m, 1H), 7.20- 7.13 (m, 1H), 5.81-5.75 (m, 1H), 5.31 (s, 1H), 4.66-4.58 (m, 1H), 2.61 (s, 3H), 2.58 (s, 3H), 1.59-1.52 (m, 9H), 1.36 (s, 6H), 1.25 (s, 3H), 1.23 (s, 3H). |
| 125 | 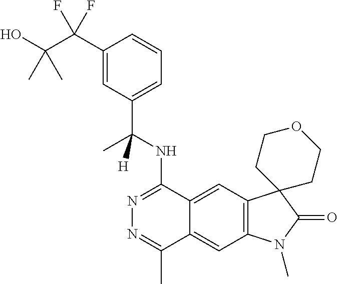 [SMILES: Cc1nnc(N[C@H](C)c2cccc(C(F)(F)C(C)(C)O)c2)c2cc3c(cc12)N(C)C(=O)C31CCOCC1] | UPLC-MS (ES⁺, Long acidic): 2.87 min, m/z 511.6 [M + H]⁺ (100%) 1H NMR (400 MHz, DMSO-d₆) 8.59 (s, 1H), 7.56-7.50 (m, 2H), 7.45 (d, J = 7.4 Hz, 1H), 7.40-7.34 (m, 2H), 7.29 (d, J = 7.6 Hz, 1H), 5.62-5.54 (m, 1H), 5.20 (s, 1H), 4.12-4.06 (m, 2H), 4.00-3.97 (m, 2H), 3.28 (s, 3H), 2.65 (s, 3H), 1.93-1.82 (m, 4H), 1.61 (d, J = 7.0 Hz, 3H), 1.13 (s, 3H), 1.07 (s, 3H) |
| 126 | 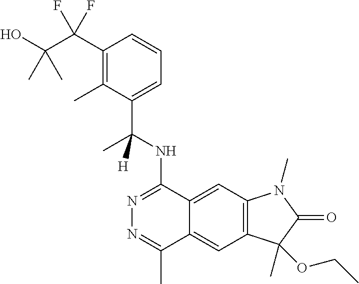 [SMILES: CCOC1(C)C(=O)N(C)c2cc3c(N[C@H](C)c4cccc(C(F)(F)C(C)(C)O)c4C)nnc(C)c3cc21] | UPLC-MS (ES⁺, Long acidic): 3.11 + 3.13 min, m/z 513.3 [M + H]⁺ (100%) 1H NMR (400 MHz, DMSO-d₆): 8.02 (s, 1H), 7.94 (s, 1H), 7.64-7.55 (m, 1H), 7.46-7.37 (m, 1H), 7.27-7.20 (m, 1H), 7.20-7.13 (m, 1H), 5.86-5.71 (m, 1H), 5.30 (s, 1H), 3.16-2.96 (m, 2H), 2.64 (s, 3H), 2.58 (s, 3H), 1.59-1.45 (m, 6H), 1.25 (s, 3H), 1.23 (s, 3H), 1.07- 0.95 (m, 3H), CH₃ under solvent peak |
| 127 | 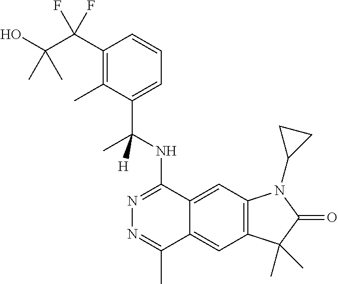 [SMILES: Cc1c([C@@H](C)Nc2nnc(C)c3cc4c(cc23)N(C2CC2)C(=O)C4(C)C)cccc1C(F)(F)C(C)(C)O] | UPLC-MS (ES⁺, Long acidic): 3.23 min, m/z 509.6 [M + H]⁺ (100%) ¹H-NMR (400 MHz, DMSO-d₆): 8.01 (s, 1H), 7.94 (s, 1H), 7.64-7.59 (m, 1H), 7.49-7.45 (m, 1H), 7.26-7.22 (m, 1H), 7.19-7.13 (m, 1H), 5.81-5.72 (m, 1H), 5.31 (s, 1H), 2.90-2.84 (m, 1H), 2.62 (s, 3H), 2.59 (s, 3H), 1.56 (d, J = 6.9 Hz, 3H), 1.35 (s, 6H), 1.24 (s, 3H), 1.23 (s, 3H), 1.18-1.14 (m, 2H), 1.00- 0.85 (m, 2H). |
| 128 | 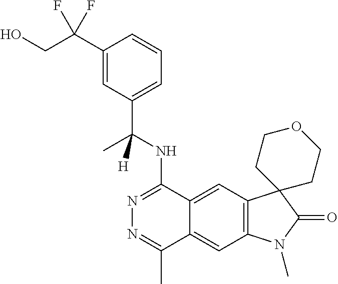 [SMILES: Cc1nnc(N[C@H](C)c2cccc(C(F)(F)CO)c2)c2cc3c(cc12)N(C)C(=O)C31CCOCC1] | UPLC-MS (ES⁺, Long acidic): 2.66 min, m/z 483.3 [M + H]⁺ (100%) ¹H-NMR (400 MHz, DMSO-d₆) 8.59 (s, 1H), 7.61 (s, 1H), 7.56 (d, J = 7.9 Hz, 1H), 7.47 (d, J = 7.0 Hz, 1H), 7.44-7.39 (m, 1H), 7.37-7.33 (m, 2H), 5.63- 5.57 (m, 2H), 4.14-4.07 (m, 2H), 4.03- 3.95 (m, 2H), 3.82 (td, J = 14.4, 6.0 Hz, 2H), 3.28 (s, 3H), 2.65 (s, 3H), 1.97- 1.81 (m, 4H), 1.61 (d, J = 6.9 Hz, 3H) |
| 129 | 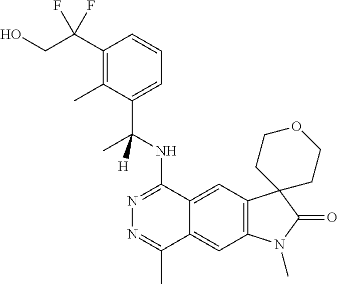 [SMILES: Cc1c([C@@H](C)Nc2nnc(C)c3cc4c(cc23)C2(CCOCC2)C(=O)N4C)cccc1C(F)(F)CO] | UPLC-MS (ES⁺, Long acidic): 2.75 min, m/z 497.5 [M + H]⁺ (100%) 1H NMR (400 MHz, DMSO-d₆) d 8.62 (s, 1H), 7.61 (d, J = 7.6 Hz, 1H), 7.49 (d, J = 7.4 Hz, 1H), 7.35-7.30 (m, 2H), 7.24-7.19 (m, 1H), 5.75-5.64 (m, 2H), 4.12-4.08 (m, 2H), 4.02-3.86 (m, 4H), 3.28 (s, 3H), 2.64 (s, 3H), 2.55 (s, 3H), 2.00-1.90 (m, 2H), 1.85-1.81 (m, 2H), 1.54 (d, J = 6.8 Hz, 3H) |
| 130 | 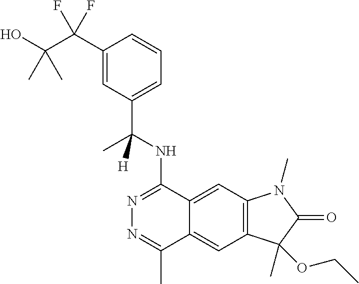 [SMILES: CCOC1(C)C(=O)N(C)c2cc3c(N[C@H](C)c4cccc(C(F)(F)C(C)(C)O)c4)nnc(C)c3cc21] | UPLC-MS (ES⁺, Long acidic): 2.95 + 2.97 min, m/z 499.5 [M + H] (100%) 1H NMR (400 MHz, DMSO-d₆): 7.99 (s, 1H), 7.95 (s, 1H), 7.57-7.49 (m, 2H), 7.43-7.32 (m, 2H), 7.33-7.26 (m, 1H), 5.55 (quintet, J = 6.8 Hz, 1H), 5.21 (s, 1H), 3.15-2.93 (m, 2H), 2.65 (s, 3H), 1.62 (d, J = 7.0 Hz, 3H), 1.54 (s, 3H), 1.12 (s, 3H), 1.09 (s, 3H), 1.07- 0.98 (m, 3H), one CH₃ under solvent peak. |
| 131 | 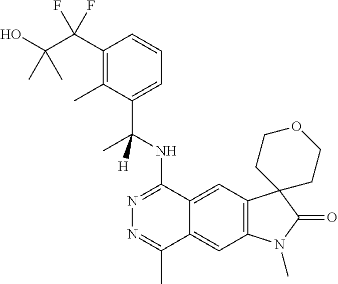 [SMILES: Cc1c([C@@H](C)Nc2nnc(C)c3cc4c(cc23)C2(CCOCC2)C(=O)N4C)cccc1C(F)(F)C(C)(C)O] | UPLC-MS (ES⁺, Long acidic): 3.02 min, m/z 525.7 [M + H]⁺ (100%) 1H NMR (400 MHz, DMSO-d₆) 8.62 (s, 1H), 7.60 (d, J = 7.5 Hz, 1H), 7.46 (d, J = 7.2 Hz, 1H), 7.34 (s, 1H), 7.24 (d, J = 7.9 Hz, 1H), 7.21-7.16 (m, 1H), 5.82-5.76 (m, 1H), 5.30 (s, 1H), 4.13- 4.06 (m, 2H), 4.04-3.96 (m, 2H), 3.28 (s, 3H), 2.64 (s, 3H), 2.59 (s, 3H), 2.00- 1.91 (m, 2H), 1.87-1.81 (m, 2H), 1.54 (d, J = 6.8 Hz, 3H), 1.24 (d, J = 5.3 Hz, 6H). |
| 132 | 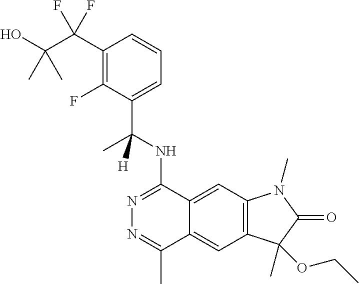 [SMILES: CCOC1(C)C(=O)N(C)c2cc3c(N[C@H](C)c4cccc(C(F)(F)C(C)(C)O)c4F)nnc(C)c3cc21] | UPLC-MS (ES⁺, Long acidic): 2.99 min, m/z 517.6 [M + H]⁺ (100%) 1H NMR (400 MHz, DMSO-d₆ 8.03 (s, 1H), 7.96 (s, 1H), 7.58-7.48 (m, 1H), 7.48-7.39 (m, 1H), 7.33-7.23 (m, 1H), 7.20-7.09 (m, 1H), 5.74 (quintet, J = 6.7 Hz, 1H), 5.35 (s, 1H), 3.34 (s, 3H), 3.13-2.94 (m, 2H), 2.64 (s, 3H), 1.61 (d, J = 7.0 Hz, 3H), 1.54 (s, 3H), 1.24 (s, 3H), 1.23 (s, 3H), 1.1-0.96 (m, 3H) |
| 133 | 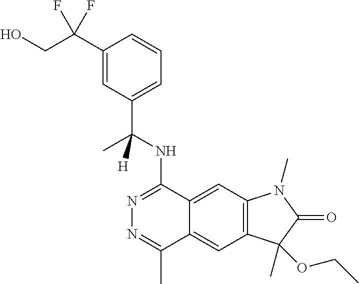 [SMILES: CCOC1(C)C(=O)N(C)c2cc3c(N[C@H](C)c4cccc(C(F)(F)CO)c4)nnc(C)c3cc21] | UPLC-MS (ES⁺, Long acidic): 2.74 min, m/z 471.5 [M + H]⁺ (100%) 1H NMR (400 MHz, DMSO-d₆): 8.00 (s, 1H), 7.95 (s, 1H), 7.63-7.58 (m, 1H), 7.58-7.51 (m, 1H), 7.48-7.37 (m, 2H), 7.37-7.30 (m, 1H), 5.66-5.47 (m, 2H), 3.91-3.73 (m, 2H), 3.15-2.95 (m, 2H), 2.65 (s, 3H), 2.65 (s, 3H), 1.61 (d, J = 7.0 Hz, 3H), 1.54 (s, 3H), 1.08- .98 (m, 3H) |
| 134 | 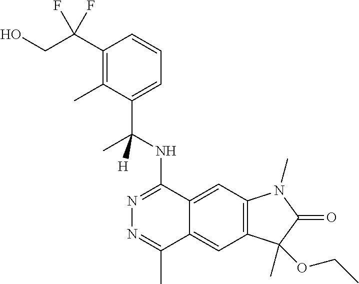 [SMILES: CCOC1(C)C(=O)N(C)c2cc3c(N[C@H](C)c4cccc(C(F)(F)CO)c4C)nnc(C)c3cc21] | UPLC-MS (ES⁺, Long acidic): 2.86 min, m/z 485.4 [M + H]⁺ (100%) 1H NMR (400 MHz, DMSO-d₆): 8.02 (s, 1H), 7.94 (s, 1H), 7.65-7.56 (m, 1H), 7.48-7.39 (m, 1H), 7.35-7.27 (m, 1H), 7.25-7.15 (m, 1H), 5.82-5.59 (m, 2H), 4.03-3.78 (m, 2H), 3.32 (s, 3H), 3.15-2.92 (m, 2H), 2.64 (s, 3H), 2.53 (s, 3H), 1.61-1.46 (m, 6H), 1.10-0.94 (m, 3H) |
| 135 | 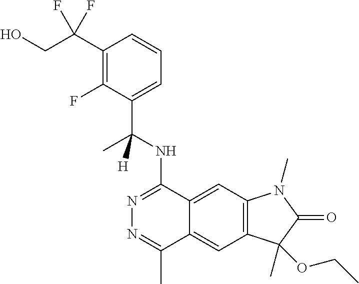 [SMILES: CCOC1(C)C(=O)N(C)c2cc3c(N[C@H](C)c4cccc(C(F)(F)CO)c4F)nnc(C)c3cc21] | UPLC-MS (ES⁺, Long acidic): 2.78 min, m/z 489.6 [M + H]⁺ (100%) 1H NMR (400 MHz, DMSO-d₆): 8.03 (s, 1H), 7.96 (s, 1H), 7.60-7.51 (m, 1H), 7.50-7.43 (m, 1H), 7.43-7.34 (m, 1H), 7.25-7.13 (m, 1H), 5.81-6.65 (m, 2H), 4.06-3.81 (m, 2H), 3.34 (s, 3H), 3.17-2.91 (m, 2H), 2.65 (s, 3H), 1.62 (d, J = 6.9 Hz, 3H), 1.54 (s, 3H), 1.08- 0.98 (m, 3H) |
| 136 | 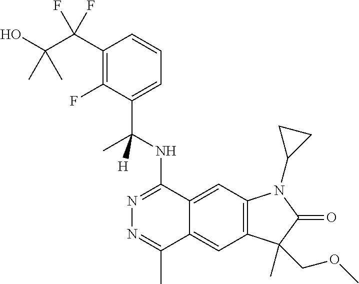 [SMILES: COCC1(C)C(=O)N(C2CC2)c2cc3c(N[C@H](C)c4cccc(C(F)(F)C(C)(C)O)c4F)nnc(C)c3cc21] | UPLC-MS (ES⁺, Long acidic): 3.10 min, m/z 543.6 [M + H]⁺ (100%) 1H NMR (400 MHz, DMSO-d₆): 8.00- 7.98 (m, 1H), 7.97 (s, 1H), 7.58-7.46 (m, 2H), 7.30-7.25 (m, 1H), 7.17-7.11 (m, 1H), 5.76-5.69 (m, 1H), 5.35 (s, 1H), 3.81-3.78 (m, 1H), 3.63-3.60 (m, 1H), 3.06 (s, 3H), 2.88-2.82 (m, 1H), 2.62 (s, 3H), 1.60 (d, J = 7.3 Hz, 3H), 1.28 (s, 3H), 1.12 (s, 3H), 1.16 (s, 3H), 1.18- 1.16 (m, 2H), 0.95-0.83 (m, 2H). |
| 137 | 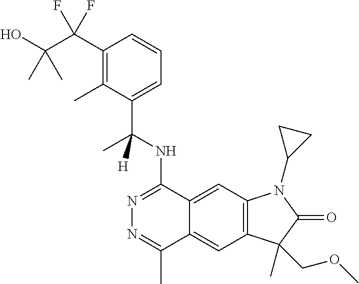 [SMILES: COCC1(C)C(=O)N(C2CC2)c2cc3c(N[C@H](C)c4cccc(C(F)(F)C(C)(C)O)c4C)nnc(C)c3cc21] | UPLC-MS (ES⁺, Long acidic): 3.22 min, m/z 539.5 [M + H]⁺ (100%) 1H NMR (400 MHz, DMSO-d₆) 7.99- 7.98 (m, 1H), 7.95 (s, 1H), 7.62-7.60 (m, 1H), 7.48-7.43 (m, 1H), 7.24-7.22 (m, 1H), 7.18-7.13 (m, 1H), 5.80-5.73 (m, 1H), 5.31 (s, 1H), 3.80-3.78 (m, 1H), 3.62-3.59 (m, 1H), 3.05 (s, 3H), 2.88- 2.82 (m, 1H), 2.62 (s, 3H), 2.59 (s, 3H), 1.56 (d, J = 7.6 Hz, 3H), 1.27 (s, 3H), 1.35 (s, 3H), 1.23 (s, 3H), 1.17-1.14 (m, 2H), 0.94-0.91 (m, 2H). |
| 138 | 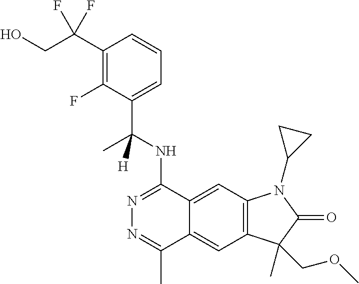 [SMILES: COCC1(C)C(=O)N(C2CC2)c2cc3c(N[C@H](C)c4cccc(C(F)(F)CO)c4F)nnc(C)c3cc21] | UPLC-MS (ES⁺, Long acidic): 2.90 min, m/z 515.7 [M + H]⁺ (100%) 1H NMR (400 MHz, DMSO-d₆): 7.99- 7.98 (m, 1H), 7.97 (s, 1H), 7.58-7.53 (m, 1H), 7.53-7.48 (m, 1H), 7.39- 7.36 (m 1H), 7.20-7.17 (m, 1H), 5.77- 5.72 (m, 2H) 4.00-3.91 (m, 2H), 3.81- 3.78 (m, 1H), 3.63-3.61 (m, 1H), 3.06 (s, 3H), 2.88-2.82 (s, 1H), 2.63 (s, 3H), 1.62 (d, J = 6.9 Hz, 3H), 1.28 (s, 3H), 1.25-1.17 (m, 2H), 0.96-0.82 (m, 2H). |
| 139 | 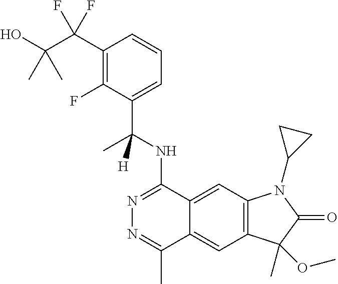 [SMILES: COC1(C)C(=O)N(C2CC2)c2cc3c(N[C@H](C)c4cccc(C(F)(F)C(C)(C)O)c4F)nnc(C)c3cc21] | UPLC-MS (ES⁺, Long acidic): 3.04 min, m/z 529.6 [M + H]⁺ (100%) 1H NMR (400 MHz, DMSO-d₆): 8.06 (s, 1H), 7.95 (d, J = 1.6 Hz, 1H), 7.64- 7.47 (m, 2H), 7.32-7.24 (m, 1H), 7.20- 7.10 (m, 1H), 5.74 (quintet, J = 7.1 Hz, 1H), 5.35 (app d, J = 0.8 Hz, 1H), 2.96- 2.83 (m, 4H), 2.64 (s, 3H), 1.62 (d, J = 7.0 Hz, 3H), 1.51 (s, 3H), 1.24 (s, 3H), 1.23 (s, 3H), 1.21-1.12 (m, 2H), 1.07- 0.78 (m, 2H) |
| 140 | 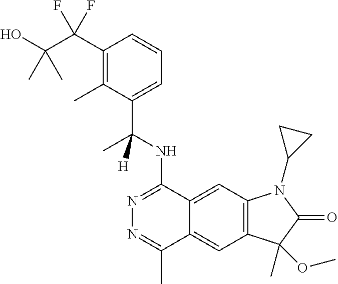 [SMILES: COC1(C)C(=O)N(C2CC2)c2cc3c(N[C@H](C)c4cccc(C(F)(F)C(C)(C)O)c4C)nnc(C)c3cc21] | UPLC-MS (ES⁺, Long acidic): 3.16 min, m/z 525.6 [M + H]⁺ (100%) 1H NMR (400 MHz, DMSO-d₆): 8.06 (s, 1H), 7.94-7.90 (m, 1H), 7.65-7.58 (m, 1H), 7.58-7.50 (m, 1H), 7.27-7.20 (m, 1H), 7.20-7.12 (m, 1H), 5.77 (quintet, J = 7.0 Hz, 1H), 5.30 (s, 1H), 2.96-2.81 (m, 4H), 2.64 (s, 3H), 2.58 (s, 3H), 1.56 (d, J = 6.9 Hz, 3H), 1.49 (s, 3H), 1.25 (s, 3H), 1.23 (s, 3H), 1.20- 1.11 (m, 2H), 1.06-0.80 (m, 2H) |
| 141 | 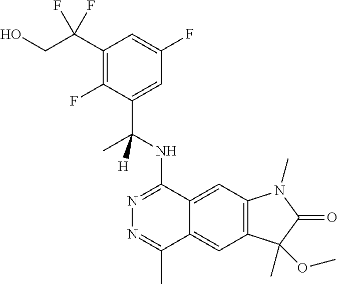 [SMILES: COC1(C)C(=O)N(C)c2cc3c(N[C@H](C)c4cc(F)cc(C(F)(F)CO)c4F)nnc(C)c3cc21] | UPLC-MS (ES⁺, Long acidic): 2.78 min, m/z 493.5 [M + H]⁺ (100%) 1H NMR (400 MHz, DMSO-d₆): 8.00- 7.98 (m, 2H), 7.47 (d, J = 6.9 Hz, 1H), 7.42-7.34 (m, 1H), 7.24-7.19 (m, 1H), 5.81 (t, J = 6.5 Hz, 1H), 5.73-5.66 (m, 1H), 4.02-3.91 (m, 2H), 3.35 (s, 3H), 2.93-2.92 (m, 3H), 2.66 (s, 3H), 1.62 (d, J = 7.0 Hz, 3H), 1.55 (s, 3H). |
| 142 | 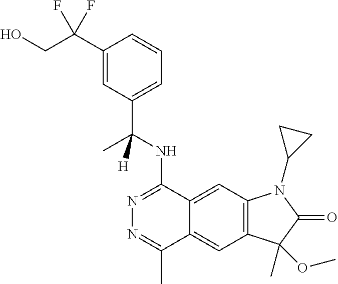 [SMILES: COC1(C)C(=O)N(C2CC2)c2cc3c(N[C@H](C)c4cccc(C(F)(F)CO)c4)nnc(C)c3cc21] | UPLC-MS (ES⁺, Long acidic): 2.78 min, m/z 483.6 [M + H]⁺ (100%) 1H NMR (400 MHz, DMSO-d₆): 8.06- 8.01 (m, 1H), 7.96-7.92 (m, 1H), 7.66- 7.60 (m, 1H), 7.60-7.52 (m, 2H), 7.46- 7.37 (m, 1H), 7.37-7.31 (m, 1H), 5.65- 5.52 (m, 2H), 3.91-3.70 (m, 2H), 2.95- 2.81 (m, 4H), 2.65 (s, 3H), 1.63 (d, J = 7.0 Hz, 3H), 1.53-1.46 (m, 3H), 1.24- 1.10 (m, 2H), 1.06-0.81 (m, 2H) |
| 143 | 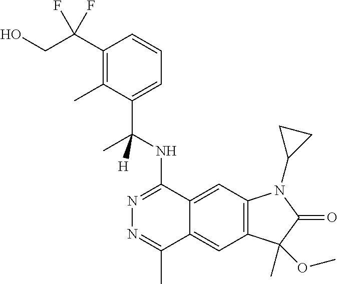 [SMILES: COC1(C)C(=O)N(C2CC2)c2cc3c(N[C@H](C)c4cccc(C(F)(F)CO)c4C)nnc(C)c3cc21] | UPLC-MS (ES⁺, Long acidic): 2.87 min, m/z 497.6 [M + H]⁺ (100%) 1H NMR (400 MHz, DMSO-d₆): 8.06 (s, 1H), 7.94-7.89 (m, 1H), 7.68-7.60 (m, 1H), 7.60-7.54 (m, 1H), 7.35-7.29 (m, 1H), 7.26-7.15 (m, 1H), 5.81-5.61 (m, 2H), 4.0-3.77 (m, 2H), 2.96-2.80 (m, 4H), 2.63 (s, 3H), 2.54 (s, 3H), 1.56 (d, J = 7.0 Hz, 3H), 1.50 (m, 3H), 1.24- 1.10 (m, 2H), 1.06-0.81 (m, 2H |
| 162 | 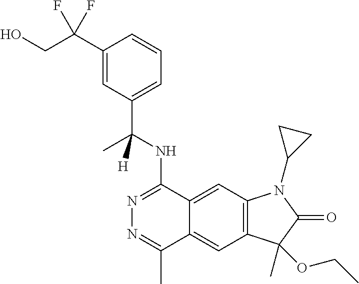 [SMILES: CCOC1(C)C(=O)N(C2CC2)c2cc3c(N[C@H](C)c4cccc(C(F)(F)CO)c4)nnc(C)c3cc21] | UPLC-MS (ES⁺, Long acidic): 2.90 min, m/z 497.5 [M + H]⁺ (100%) 1H NMR (400 MHz, DMSO-d₆): 8.02 (s, 1H), 7.93 (s, 1H), 7.67-7.60 (m 1H), 7.60-7.51 (m, 2H), 7.44-7.38 (m, 1H), 7.38-7.31 (m, 1H), 5.64-5.52 (m, 2H), 3.90-3.74 (m, 2H), 3.13-2.92 (m, 2H), 2.92-2.81 (m, 1H), 2.65 (s, 3H), 1.63 (d, J = 7.0 Hz, 3H), 1.50 (s, 3H), 1.20-1.10 (m, 2H), 1.07-0.98 (m, 3H), 0.98-0.82 (m, 2H) |
| 163 | 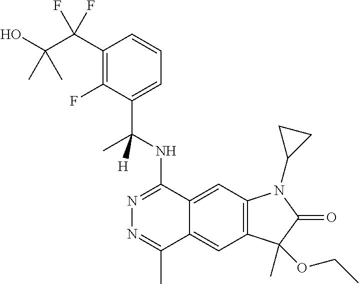 [SMILES: CCOC1(C)C(=O)N(C2CC2)c2cc3c(N[C@H](C)c4cccc(C(F)(F)C(C)(C)O)c4F)nnc(C)c3cc21] | UPLC-MS (ES⁺, Long acidic): 3.13 min, m/z 543.6 [M + H]⁺ (97%) 1H NMR (400 MHz, DMSO-d₆): 8.05 (s, 1H), 7.93 (s, 1H), 7.63-7.46 (m, 2H), 7.33-7.23 (m, 1H), 7.19-7.11 (m, 1H), 5.80-5.68 (m, 1H), 5.35 (s, 1H), 3.11-2.94 (m, 2H), 2.93-2.84 (m, 1H), 2.64 (s, 3H), 1.62 (d, J = 7.0 Hz, 3H), 1.50 (s, 3H), 1.24 (s, 3H), 1.23 (s, 3H), 1.20-1.11 (m, 2H), 1.07-0.99 (m, 3H), 0.99-0.82 (m, 2H) |
| 164 | 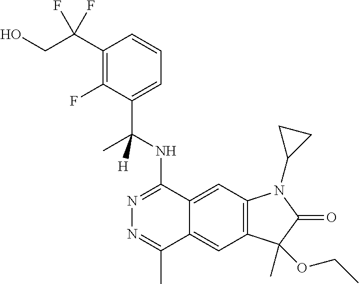 [SMILES: CCOC1(C)C(=O)N(C2CC2)c2cc3c(N[C@H](C)c4cccc(C(F)(F)CO)c4F)nnc(C)c3cc21] | UPLC-MS (ES⁺, Long acidic): 2.93 + 2.94 min, m/z 515.5 [M + H]⁺ (38 + 62%) 1H NMR (400 MHz, DMSO-d₆): 8.06 (s, 1H), 7.94 (s, 1H), 7.65-7.53 (m, 2H), 7.44-7.34 (m, 1H), 7.25-7.14 (m, 1H), 5.79-5.69 (m, 2H), 4.05-3.88 (m, 2H), 3.12-2.93 (m, 2H), 2.93-2.84 (m, 1H), 2.64 (s, 3H), 1.63 (d, J = 7.0 Hz, 3H), 1.50 (s, 3H), 1.24-1.11 (m, 2H), 1.07-0.99 (m, 3H), 0.99-0.83 (m, 2H) |
| 165 | 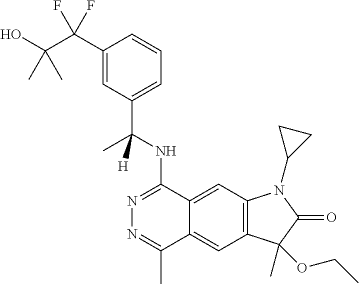 [SMILES: CCOC1(C)C(=O)N(C2CC2)c2cc3c(N[C@H](C)c4cccc(C(F)(F)C(C)(C)O)c4)nnc(C)c3cc21] | UPLC-MS (ES⁺, Long acidic): 3.11 min, m/z 525.6 [M + H]⁺ (100%) 1H NMR (400 MHz, DMSO-d₆): 8.01 (s, 1H), 7.93 (s, 1H), 7.58-7.49 (m, 1H), 5.65-5.51 (m, 1H), 5.21 (s, 1H), 3.11-2.93 (m, 2H), 2.93-2.84 (m, 1H), 2.64 (s, 3H), 1.64 (d, J = 7.0 Hz, 3H), 1.50 (s, 3H), 1.20-1.13 (m, 2H), 1.12 (s, 3H), 1.11-1.06 (m, 3H), 1.05-0.98 (m, 3H), 0.98-0.81 (m, 2H) |
| 166 | 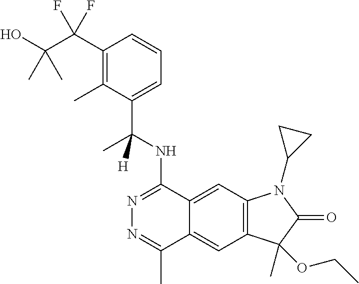 [SMILES: CCOC1(C)C(=O)N(C2CC2)c2cc3c(N[C@H](C)c4cccc(C(F)(F)C(C)(C)O)c4C)nnc(C)c3cc21] | UPLC-MS (ES⁺, Long acidic): 3.24 + 3.26 min, m/z 539.6 [M + H]⁺ (39 + 61%) 1H NMR (400 MHz, DMSO-d₆): 8.05 (s, 1H), 7.92 (s, 1H), 7.65-7.58 (m, 1H), 7.57-7.50 (m, 1H), 7.26-7.21 (m, 1H), 7.20-7.12 (m, 1H), 5.82-5.70 (m, 1H), 5.30 (s, 1H), 3.10-2.92 (m, 2H), 2.92-2.84 (m, 1H), 2.63 (s, 3H), 2.58 (s, 3H), 1.56 (d, J = 7.0 Hz, 3H), 1.49 (s, 3H), 1.25 (s, 3H), 1.23 (s, 3H), 1.20- 1.12 (m, 2H), 1.06-0.98 (m, 3H), 0.98- 0.83 (m, 2H) |
| 167 | 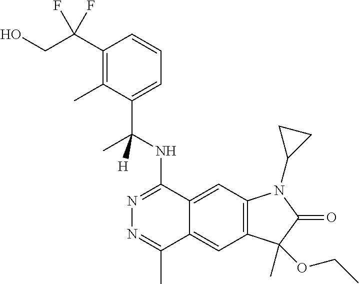 [SMILES: CCOC1(C)C(=O)N(C2CC2)c2cc3c(N[C@H](C)c4cccc(C(F)(F)CO)c4C)nnc(C)c3cc21] | UPLC-MS (ES⁺, Long acidic): 3.00 + 3.01 min, m/z 511.5 [M + H]⁺ (43 + 57%) 1H NMR (400 MHz, DMSO-d₆): 8.05 (s, 1H), 7.91 (s, 1H), 7.66-7.59 (m, 1H), 7.59-7.53 (m, 1H), 7.35-7.28 (m, 1H), 7.24-7.15 (m, 1H), 5.79-5.69 (m, 1H), 5.69-5.62 (m, 1H), 3.99-3.83 (m, 2H), 3.12-2.92 (m, 2H), 2.93-2.83 (m, 1H), 2.63 (s, 3H), 2.54 (s, 3H), 1.56 (d, J = 7.0 Hz, 3H), 1.49 (s, 3H), 1.22-1.11 (m, 2H), 1.06-0.98 (m, 3H), 0.98-0.80 (m, 2H) |
| 168 | 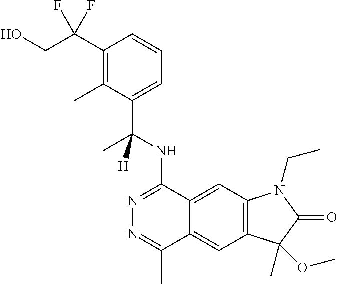 [SMILES: CCN1C(=O)C(C)(OC)c2cc3c(C)nnc(N[C@H](C)c4cccc(C(F)(F)CO)c4C)c3cc21] | UPLC-MS (ES⁺, Long acidic): 2.87 min, m/z 485.5 [M + H]⁺ (100%) ¹H-NMR (400 MHz, DMSO-d₆): 8.04 (s, 1H), 7.96 (s, 1H), 7.64-7.59 (m, 1H), 7.48-7.43 (m, 1H), 7.35-7.30 (m, 1H), 7.24-7.18 (m, 1H), 5.78-5.68 (m, 2H), 3.97-3.84 (m, 4H), 2.91-2.90 (m, 3H), 2.64 (s, 3H), 2.53-2.53 (m, 3H), 1.56 (d, J = 6.9 Hz, 3H), 1.53 (m, 3H), 1.30 (t J = 7.1 Hz, 3H). |
| 169 | 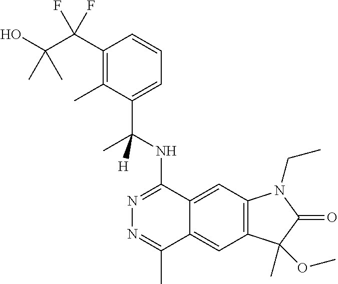 [SMILES: CCN1C(=O)C(C)(OC)c2cc3c(C)nnc(N[C@H](C)c4cccc(C(F)(F)C(C)(C)O)c4C)c3cc21] | UPLC-MS (ES⁺, Long acidic): 3.15 min, m/z 513.6 [M + H]⁺ (100%) ¹H-NMR (400 MHz, DMSO-d₆): 8.05 (s, 1H), 7.96 (s, 1H), 7.82-7.57 (m, 1H), 7.45-7.41 (m, 1H), 7.26-7.23 (m, 1H), 7.20-7.15 (m, 1H), 5.81-5.72 (m, 1H), 5.33 (s, 1H), 3.95-3.85 (m, 2H), 2.91-2.89 (m, 3H), 2.64 (s, 3H), 2.60- 2.57 (m, 3H), 1.56 (d, J = 6.8 Hz, 3H), 1.53-1.52 (m, 3H), 1.30 (t, J = 7.2 Hz, 3H), 1.24 (s, 3H), 1.23 (s, 3H). |
| 170 | 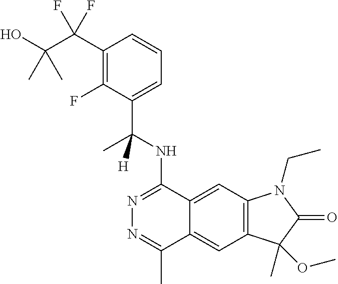 [SMILES: CCN1C(=O)C(C)(OC)c2cc3c(C)nnc(N[C@H](C)c4cccc(C(F)(F)C(C)(C)O)c4F)c3cc21] | UPLC-MS (ES⁺, Long acidic): 3.03 min, m/z 517.6 [M + H]⁺ (100%) ¹H-NMR (400 MHz, DMSO-d₆): 8.05 (s, 1H), 7.82-7.80 (m, 1H)*, 7.56-7.50 (m, 1H), 7.45 (d, J = 7.3 Hz, 1H), 7.32- 7.26 (m, 1H), 7.19-7.13 (m, 1H), 5.77- 5.69 (m, 1H), 5.38 (s, 1H), 3.94-3.86 (m, 2H), 2.92-2.91 (m, 3H), 2.64 (s, 3H), 1.62 (d, J - 6.9 Hz, 3H), 1.53-1.52 (m, 3H), 1.31 (t, J = 7.1 Hz, 3H), 1.24 (s, 3H), 1.23 (s, 3H) |
| 171 | 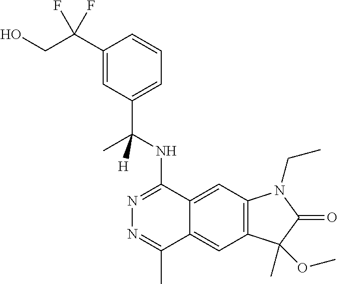 [SMILES: CCN1C(=O)C(C)(OC)c2cc3c(C)nnc(N[C@H](C)c4cccc(C(F)(F)CO)c4)c3cc21] | UPLC-MS (ES⁺, Long acidic): 2.79 min, m/z 471.5 [M + H]⁺ (100%) ¹H-NMR (400 MHz, DMSO-d₆): 8.02- 8.01 (m, 1H), 7.97 (s, 1H), 7.61 (br d, J = 6.7 Hz, 1H), 7.58-7.53 (m, 1H), 7.44- 7.39 (m, 2H), 7.34 (br d, J = 7.8 Hz, 1H), 5.63-5.54 (m, 2H), 3.96-3.76 (m, 4H), 2.91-2.90 (m, 3H), 2.65 (s, 3H), 1.63 (d, J = 6.9 Hz, 3H), 1.53 (s, 3H), 1.29 (t, J = 7.2 Hz, 3H). |
| 172 | 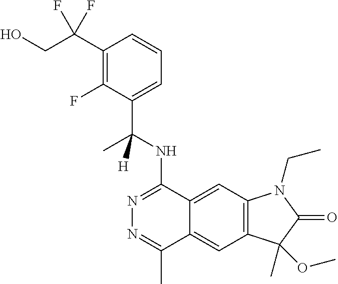 [SMILES: CCN1C(=O)C(C)(OC)c2cc3c(C)nnc(N[C@H](C)c4cccc(C(F)(F)CO)c4F)c3cc21] | UPLC-MS (ES⁺, Long acidic): 2.82 min, m/z 489.5 [M + H]⁺ (100%) ¹H-NMR (400 MHz, DMSO-d₆): 8.06 (s, 1H), 7.99 (s, 1H), 7.60-7.53 (m, 1H), 7.46 (d, J = 7.2 Hz, 1H), 7.42-7.36 (m, 1H), 7.23-7.17 (m, 1H), 5.78-5.70 (m, 2H), 4.01-3.84 (m, 4H), 2.92-2.92 (m, 3H), 2.64 (s, 3H), 1.63 (d, J = 7.0 Hz, 3H), 1.54-1.53 (m, 3H), 1.31 (d, J = 7.1 Hz, 3H) |
| 173 | 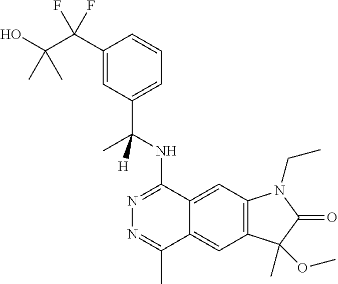 [SMILES: CCN1C(=O)C(C)(OC)c2cc3c(C)nnc(N[C@H](C)c4cccc(C(F)(F)C(C)(C)O)c4)c3cc21] | UPLC-MS (ES⁺, Long acidic): 2.99 min, m/z 499.6 [M + H]⁺ (100%) ¹H-NMR (400 MHz, DMSO-d₆): 8.02 (s, 1H), 7.97-7.96 (m, 1H), 7.56-7.50 (m, 2H), 7.42-7.34 (m, 2H), 7.32-7.27 (m, 1H), 5.63-5.52 (m, 1H), 5.21-5.20 (m, 1H), 3.97-3.79 (m, 2H), 2.91-2.90 (m, 3H), 2.65 (s, 3H), 1.63 (d, J = 7.0 Hz, 3H), 1.53 (m, 3H)*, 1.29 (t, J = 7.2 Hz, 3H), 1.12 (s, 3H), 1.08-1.07 (m, 3H) |
| 174 | 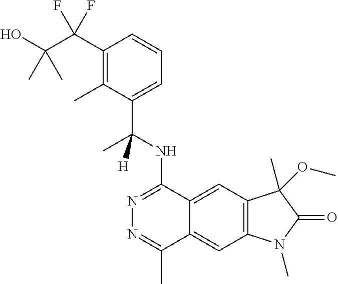 [SMILES: COC1(C)C(=O)N(C)c2cc3c(C)nnc(N[C@H](C)c4cccc(C(F)(F)C(C)(C)O)c4C)c3cc21] | UPLC-MS (ES⁺, Long acidic): 3.03 min, m/z 499.5 [M + H]⁺ (97%) 1H NMR (400 MHz, DMSO-d₆): 8.60 (d, J = 9.1 Hz, 1H), 7.65-7.50 (m, 2H), 7.40 (s, 1H), 7.14-7.25 (m, 2H), 5.74 (m, 1H), 5.30 (s, 1H), 3.30 (s, 3H), 2.94 (d, J = 4.44 Hz, 3H), 2.65 (m, 3H), 2.58 (s, 3H), 1.52-1.57 (m, 6H), 1.24 (s, 3H), 1.23 (s, 3H) |
| 175 | 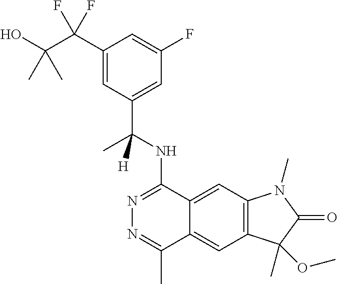 [SMILES: COC1(C)C(=O)N(C)c2cc3c(N[C@H](C)c4cc(F)cc(C(F)(F)C(C)(C)O)c4)nnc(C)c3cc21] | UPLC-MS (ES⁺, Long acidic): 3.31 min, m/z 503.2 [M + H]⁺ (100%) 1H NMR (400 MHz, DMSO-d₆): 7.98 (s, 1H), 7.97 (s, 1H), 7.47-7.41 (m, 1H), 7.41-7.30 (m, 2H), 7.12-7.02 (m, 1H), 5.60-5.46 (m, 1H), 5.31 (s, 1H), 3.33 (s, 3H), 2.91 (d, J = 1.2 Hz, 3H), 2.65 (s, 3H), 1.62 (d, J = 7.0 Hz, 3H), 1.54 (s, 3H), 1.13 (s, 3H), 1.09 (s, 3H) |
| 176 | 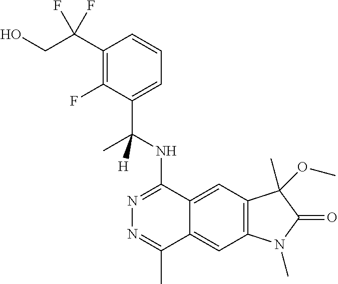 [SMILES: COC1(C)C(=O)N(C)c2cc3c(C)nnc(N[C@H](C)c4cccc(C(F)(F)CO)c4F)c3cc21] | UPLC-MS (ES⁺, Long acidic): 2.66 min, m/z 475.5 [M + H]⁺ (100%) 1H NMR (400 MHz, DMSO-d₆): 8.60- 8.58 (m, 1H), 7.61-7.52 (m, 2H), 7.43- 7.35 (m, 2H), 7.23-7.16 (m, 1H), 5.78- 5.68 (m, 2H), 4.03-3.89 (m, 2H), 3.31 (s, 3H), 2.96-2.93 (m, 3H), 2.66-2.65 (m, 3H), 1.62-1.55 (m, 6H) |
| 177 | 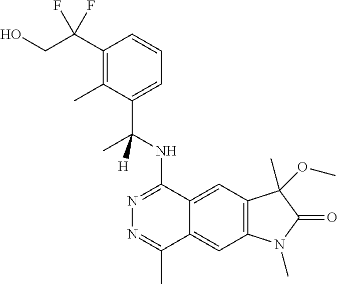 [SMILES: COC1(C)C(=O)N(C)c2cc3c(C)nnc(N[C@H](C)c4cccc(C(F)(F)CO)c4C)c3cc21] | UPLC-MS (ES⁺, Long acidic): 2.70 min, m/z 471.5 [M + H]⁺ (100%) 1H NMR (400 MHz, DMSO-d₆): 8.60- 8.58 (m, 1H), 7.62-7.52 (m, 2H), 7.39 (s, 1H), 7.34-7.29 (m, 1H), 7.25-7.17 (m, 1H), 5.75-5.64 (m, 2H), 3.97-3.85 (m, 2H), 3.30 (s, 3H), 2.94-2.93 (m, 3H), 2.65-2.64 (m, 3H), 2.55-2.54 (m, 3H), 1.58-1.51 (m, 6H) |
| 178 | 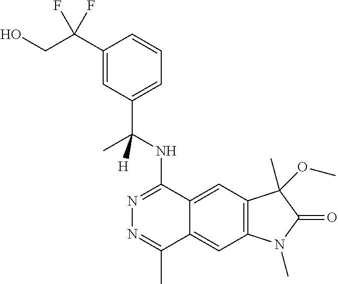 [SMILES: COC1(C)C(=O)N(C)c2cc3c(C)nnc(N[C@H](C)c4cccc(C(F)(F)CO)c4)c3cc21] | UPLC-MS (ES⁺, Long acidic): 2.60 min, m/z 457.4 [M + H]⁺ (100%) 1H NMR (400 MHz, DMSO-d₆): 8.57- 8.55 (m, 1H), 7.63-7.50 (m, 3H), 7.43- 7.37 (m, 2H), 7.36-7.31 (m, 1H), 5.63- 5.50 (m, 2H), 3.87-3.76 (m, 2H), 3.30 (s, 3H), 2.93-2.92 (m, 3H), 2.66-2.65 (m, 3H), 1.61-1.58 (m, 3H), 1.57-1.54 (m, 3H) |
| 179 | 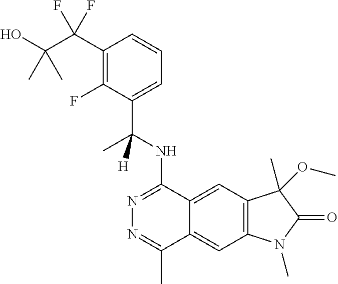 [SMILES: COC1(C)C(=O)N(C)c2cc3c(C)nnc(N[C@H](C)c4cccc(C(F)(F)C(C)(C)O)c4F)c3cc21] | UPLC-MS (ES⁺, Long acidic): 2.88 min, m/z 503.6 [M + H]⁺ (100%) 1H NMR (400 MHz, DMSO-d₆) 8.60- 8.58 (m, 1H), 7.58-7.49 (m, 2H), 7.41 (s, 1H), 7.32-7.25 (m, 1H), 7.19-7.13 (m, 1H), 5.76-5.66 (m, 1H), 5.34 (s, 1H), 3.31 (s, 3H), 2.96-2.93 (m, 3H), 2.66-2.65 (m, 3H), 1.60-1.55 (m, 6H), 1.25-1.20 (m, 6H). |
| 180 | 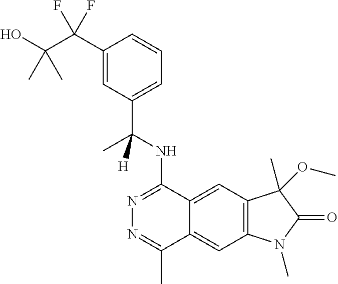 [SMILES: COC1(C)C(=O)N(C)c2cc3c(C)nnc(N[C@H](C)c4cccc(C(F)(F)C(C)(C)O)c4)c3cc21] | UPLC-MS (ES⁺, Long acidic): 2.84 min, m/z 485.6 [M + H]⁺ (100%) 1H NMR (400 MHz, DMSO-d₆): 8.57- 8.56 (m, 1H), 7.57-7.49 (m, 3H), 7.40- 7.32 (m, 2H), 7.31-7.27 (m, 1H), 5.59- 5.50 (m, 1H), 5.20-5.19 (m, 1H), 3.30 (s, 3H), 2.93-2.92 (m, 3H), 2.65 (s, 3H), 1.62-1.58 (m, 3H), 1.55 (s, 3H), 1.13-1.03 (m, 6H) |
| 181 | 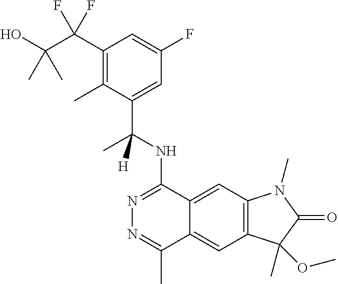 [SMILES: COC1(C)C(=O)N(C)c2cc3c(N[C@H](C)c4cc(F)cc(C(F)(F)C(C)(C)O)c4C)nnc(C)c3cc21] | UPLC-MS (ES⁺, Long acidic): 3.12 min, m/z 517.6 [M + H]⁺ (100%) 1H NMR (400 MHz, DMSO-d₆): 8.00 (s, 1H), 7.96 (s, 1H), 7.48-7.38 (m, 2H), 7.02 (dd, J = 2.8, 10.0 Hz, 1H), 5.77-5.67 (m, 1H), 5.40 (s, 1H), 3.35- 3.34 (m, 3H), 2.92-2.91 (m, 3H), 2.65 (s, 3H), 2.56 (s, 3H), 1.56-1.53 (m, 6H), 1.27-1.23 (m, 6H) |
| 182 | 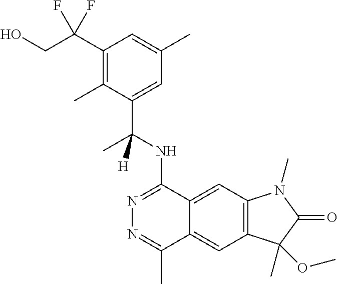 [SMILES: COC1(C)C(=O)N(C)c2cc3c(N[C@H](C)c4cc(C)cc(C(F)(F)CO)c4C)nnc(C)c3cc21] | UPLC-MS (ES⁺, Long acidic): 2.86 min, m/z 485.5 [M + H]⁺ (100%) 1H NMR (400 MHz, DMSO-d₆): 8.01 (s, 1H), 7.95 (s, 1H), 7.46-7.43 (m, 1H), 7.42-7.38 (m, 1H), 7.15 (s, 1H), 5.74-5.62 (m, 2H), 3.94-3.82 (m, 2H), 2.92 (s, 3H), 2.65 (s, 3H), 2.47 (s, 3H), 2.24-2.23 (m, 3H)*, 1.56-1.51 (m, 6H). CH₃ under solvent peak |
| 183 | 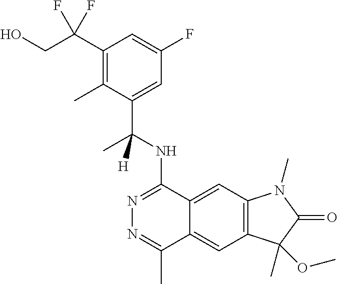 [SMILES: COC1(C)C(=O)N(C)c2cc3c(N[C@H](C)c4cc(F)cc(C(F)(F)CO)c4C)nnc(C)c3cc21] | UPLC-MS (ES⁺, Long acidic): 2.84 min, m/z 489.5 [M + H]⁺ (100%) 1H NMR (400 MHz, DMSO-d₆): 7.99 (s, 1H), 7.96 (s, 1H), 7.47 (d, J = 7.0 Hz, 1H), 7.45-7.39 (m, 1H), 7.10 (dd, J = 2.8, 9.6 Hz, 1H), 5.76-5.66 (m, 2H), 4.00-3.88 (m, 2H), 3.35-3.34 (m, 3H), 2.92-2.91 (m, 3H), 2.65 (s, 3H), 1.57- 1.52 (m, 6H). CH₃ under solvent peak |
| 184 | 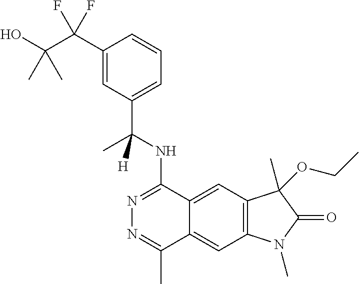 [SMILES: CCOC1(C)C(=O)N(C)c2cc3c(C)nnc(N[C@H](C)c4cccc(C(F)(F)C(C)(C)O)c4)c3cc21] | UPLC-MS (ES⁺, Long acidic): 2.94 + 2.96 min, m/z 499.8 [M + H]⁺ (38 + 63%) 1H NMR (400 MHz, DMSO-d₆): 8.58 (s, 1H), 7.57-7.49 (m, 3H), 7.38 (s, 1H), 7.37-7.31 (m, 1H), 7.31-7.25 (m, 1H), 5.62-5.47 (m, 1H), 5.21-5.17 (m, 1H), 3.29 (s, 3H), 3.12-2.93 (m, 2H), 2.65 (s, 3H), 1.65-1.57 (m, 3H), 1.55 (s, 3H), 1.13-1.09 (m, 3H), 1.09-0.98 (m, 6H) |
| 185 | 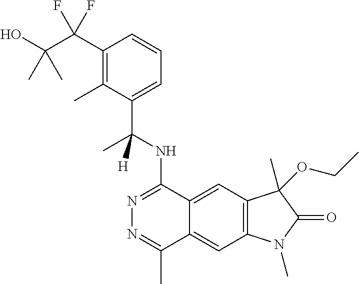 [SMILES: CCOC1(C)C(=O)N(C)c2cc3c(C)nnc(N[C@H](C)c4cccc(C(F)(F)C(C)(C)O)c4C)c3cc21] | UPLC-MS (ES⁺, Long acidic): 3.09 + 3.11 min, m/z 513.9 [M + H]⁺ (35 + 65%) 1H NMR (400 MHz, DMSO-d₆): 8.64- 8.57 (m, 1H), 7.64-7.49 (m, 2H), 7.37 (s, 1H), 7.28-7.11 (m, 2H), 5.75 (quintet, J = 7.0 Hz, 1H), 5.29 (s, 1H), 3.29 (s, 3H), 3.13-2.97 (m, 2H), 2.66- 2.63 (m, 3H), 2.60-2.56 (m, 3H), 1.59- 1.49 (m, 6H), 1.24 (s, 3H), 1.22 (s, 3H), 1.06 (t, J = 7.0 Hz, 3H) |
| 186 | 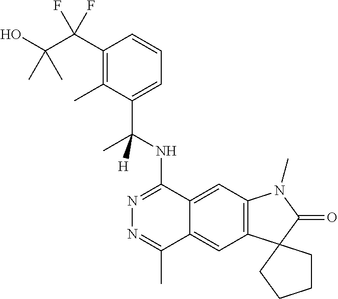 [SMILES: Cc1c([C@@H](C)Nc2nnc(C)c3cc4c(cc23)N(C)C(=O)C42CCCC2)cccc1C(F)(F)C(C)(C)O] | UPLCMS (ES⁺, long acidic): 3.02 min, m/z 509.7 [M + H]+ (100%) 1H NMR (400 MHz, DMSO-d₆): 7.95 (s, 1H), 7.80 (s, 1H), 7.58 (d, J = 7.4 Hz, 1H), 7.35 (d, J = 7.5 Hz, 1H), 7.26- 7.21 (m, 1H), 7.19-7.13 (m, 1H), 5.80- 5.72 (m, 1H), 5.32 (s, 1H), 3.31 (s, 3H), 2.63 (s, 3H), 2.58 (s, 3H), 2.09- 1.89 (m, 8H), 1.54 (d, j = 6.88 Hz, 3H), 1.24 (s, 3H), 1.23 (s, 3H) |
| 187 | 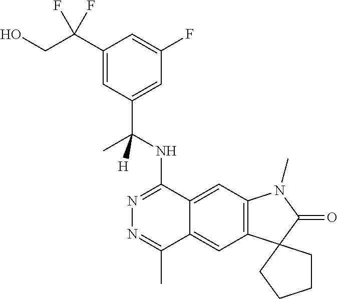 [SMILES: Cc1nnc(N[C@H](C)c2cc(F)cc(C(F)(F)CO)c2)c2cc3c(cc12)C1(CCCC1)C(=O)N3C] | UPLC-MS (ES⁺, Long acidic): 3.03 min, m/z 485.6 [M + H]⁺ (100%) 1H NMR (400 MHz, DMSO-d₆): 7.90 (s, 1H), 7.82 (s, 1H), 7.46 (s, 1H), 7.40- 7.34 (m, 2H), 7.20-7.15 (m, 1H), 5.65 (t, J = 6.3 Hz, 1H), 5.56-5.48 (m, 1H), 3.88-3.80 (td, J = 6.44/14.24 Hz, 2H), 3.31 (s, 3H), 2.64 (s, 3H), 2.06-1.92 (m, 8H), 1.61 (d, J = 7.0 Hz, 3H). |
| 188 | 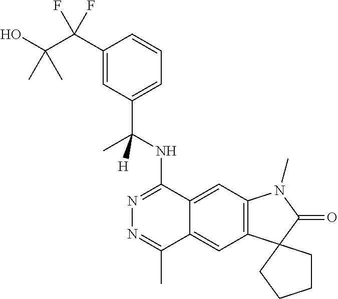 [SMILES: Cc1nnc(N[C@H](C)c2cccc(C(F)(F)C(C)(C)O)c2)c2cc3c(cc12)C1(CCCC1)C(=O)N3C] | UPLC-MS (ES⁺, Long acidic): 3.11 min, m/z 495.8 [M + H]⁺ (100%) 1H NMR (400 MHz, DMSO-d₆): 7.92 (s, 1H), 7.81 (s, 1H), 7.52-7.50 (m, 2H), 7.37-7.32 (m, 2H), 7.30-7.28 (m, 1H), 5.57-5.50 (m, 1H), 5.21 (s, 1H), 3.31 (s, 3H), 2.63 (s, 3H), 2.06-1.91 (m, 8H), 1.61 (d, J = 7.0 Hz, 3H), 1.12 (s, 3H), 1.09 (s, 3H) |
| 189 | 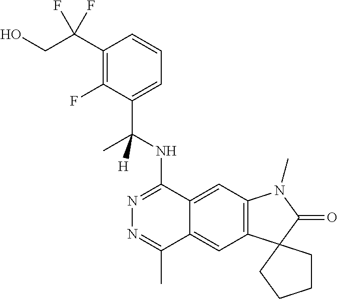 [SMILES: Cc1nnc(N[C@H](C)c2cccc(C(F)(F)CO)c2F)c2cc3c(cc12)C1(CCCC1)C(=O)N3C] | UPLC-MS (ES⁺, Long acidic): 2.97 min, m/z 485.6 [M + H]⁺ (100%) 1H NMR (400 MHz, DMSO-d6, WH- N203678-38-01_1HNMR): 7.96 (s, 1H), 7.82 (s, 1H), 7.57-7.51 (m, 1H), 7.40-7.36 (m, 2H), 7.21-7.15 (m, 1H), 5.76-5.70 (m, 2H), 4.00-3.91 (m, 2H), 2.62 (s, 3H), 2.01-1.91 (m, 8H), 1.61 (d, J = 7.0 Hz, 3H), CH₃ under solvent peak |
| 190 | 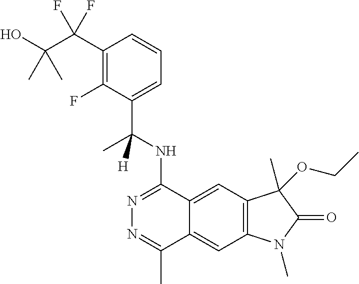 [SMILES: CCOC1(C)C(=O)N(C)c2cc3c(C)nnc(N[C@H](C)c4cccc(C(F)(F)C(C)(C)O)c4F)c3cc21] | UPLC-MS (ES⁺, Long acidic): 3.01 min, m/z 517.7 [M + H]⁺ (100%) 1H NMR (400 MHz, DMSO-d₆): 8.66- 8.59 (m, 1H), 7.61-7.47 (m, 2H), 7.40 (s, 1H), 7.33-7.22 (m, 1H), 7.21- 7.11 (m, 1H), 5.80-5.64 (m, 1H), 5.34 (s, 1H), 3.30 (s, 3H), 3.14-2.99 (m, 2H), 2.68-2.61 (m, 3H), 1.61-1.52 (m, 6H), 1.27-1.17 (m, 6H), 1.11-1.01 (m, 3H) |
| 191 | 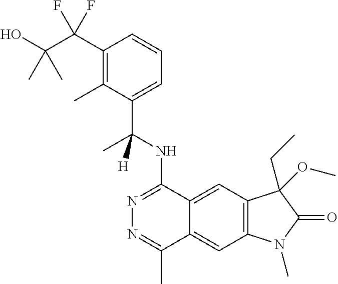 [SMILES: CCC1(OC)C(=O)N(C)c2cc3c(C)nnc(N[C@H](C)c4cccc(C(F)(F)C(C)(C)O)c4C)c3cc21] | UPLC-MS (ES⁺, Long acidic): 3.14 min, m/z 513.8 [M + H]⁺ (61 + 39%)- mixture of diastereomers 1H NMR (400 MHz, DMSO-d₆) 8.57- 8.54 (m, 1H), 7.62-7.54 (m, 2H), 7.39 (s, 1H), 7.25-7.13 (m, 2H), 5.77-5.71 (m, 1H), 5.29 (s, 1H), 2.85 (s, 3H), 2.96- 2.94 (m, 3H), 2.66-2.63 (m, 3H), 2.60- 2.57 (m, 3H), 2.02-1.96 (m, 2H), 1.54- 1.51 (m, 3H), 1.24 (s, 3H), 1.22 (s, 3H), 0.69-0.63 (m, 3H). |
| 192 | 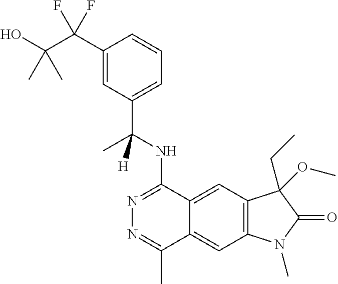 [SMILES: CCC1(OC)C(=O)N(C)c2cc3c(C)nnc(N[C@H](C)c4cccc(C(F)(F)C(C)(C)O)c4)c3cc21] | UPLC-MS (ES⁺, Long acidic, AD- N203738-30-01_UPLCMS-LONG- SQD): 2.98 min, m/z 499.8 [M + H]⁺ (48 + 52%) mixture of Diastereomers 1H NMR (400 MHz, DMSO-d₆): 8.53- 8.52 (m, 1H), 7.56-7.54 (m, 3H), 7.40 (s, 1H), 7.36-7.32 (m, 1H), 7.29-7.27 (m, 1H), 5.57-5.50 (m, 1H), 5.19 (s, 1H), 3.31 (s, 3H), 2.94-2.92 (m, 3H), 2.66 (s, 3H), 2.02-1.94 (m, 2H), 1.61-1.58 (m, 3H), 1.10 (s, 3H), 1.03 (s, 3H), 0.65 (t, J = 7.8 Hz, 3H). |
| 193 | 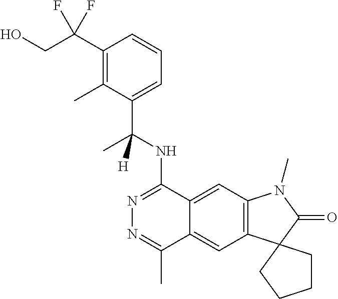 [SMILES: Cc1c([C@@H](C)Nc2nnc(C)c3cc4c(cc23)N(C)C(=O)C42CCCC2)cccc1C(F)(F)CO] | UPLC-MS (ES⁺, Long acidic): 3.05 min, m/z 481.6 [M + H]⁺ (100%) 1H NMR (400 MHz, DMSO-d₆): 7.95 (s, 1H), 7.80 (s, 1H), 7.60 (d, J = 7.88 Hz, 1H), 7.34 (d, J = 7.88 Hz, 1H), 7.31 (d, J = 7.31 Hz, 1H), 7.22-7.16 (m, 1H), 5.74-5.67 (m, 2H), 3.95-3.86 (m, 2H), 3.31 (s, 3H), 2.62 (s, 3H), 2.05-1.89 (m, 8H), 1.54 (d, J = 6.88 Hz, 3H), CH₃ under solvent peak |
| 194 | 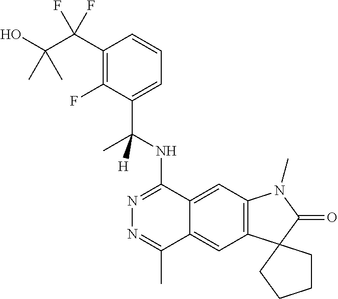 [SMILES: Cc1nnc(N[C@H](C)c2cccc(C(F)(F)C(C)(C)O)c2F)c2cc3c(cc12)C1(CCCC1)C(=O)N3C] | UPLC-MS (ES⁺, Long acidic): 3.19 min, m/z 513.8 [M + H]⁺ (100%) 1H NMR (400 MHz, DMSO-d₆): 7.96 (s, 1H), 7.82 (s, 1H), 7.54-7.48 (m, 1H), 7.32 (d, J = 7.38 Hz, 1H), 7.30- 7.25 (m, 1H), 7.17-7.11 (m, 1H), 5.77- 5.68 (m, 1H), 5.34 (s, 1H), 2.63 (s, 3H), 2.63 (s, 3H), 2.06-1.92 (m, 8H), 1.60 (d, J = 7.00 Hz, 3H), 1.24 (s, 3H), 1.23 (s, 3H). |
| 195 | 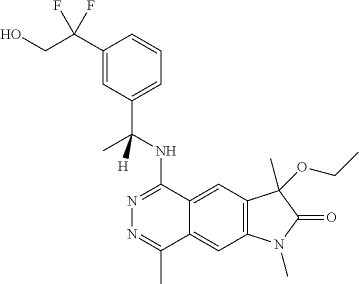 [SMILES: CCOC1(C)C(=O)N(C)c2cc3c(C)nnc(N[C@H](C)c4cccc(C(F)(F)CO)c4)c3cc21] | UPLC-MS (ES⁺, Long acidic): 2.77 min, m/z 471.6 [M + H]⁺ (100%) 1H NMR (400 MHz, DMSO-d₆): 8.61- 8.54 (m, 1H), 7.64-7.49 (m, 3H), 7.45- 7.36 (m, 2H), 7.36-7.28 (m, 1H), 5.64- 5.48 (m, 2H), 3.91-3.71 (m, 2H), 3.29 (s, 3H), 3.14-2.95 (m, 2H), 2.70-2.60 (m, 3H), 1.64-1.57 (m, 3H), 1.57-1.51 (m, 3H), 1.10-1.0 (m, 3H) |
| 196 | 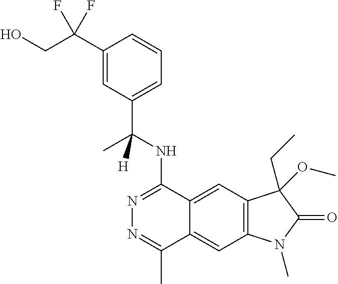 [SMILES: CCC1(OC)C(=O)N(C)c2cc3c(C)nnc(N[C@H](C)c4cccc(C(F)(F)CO)c4)c3cc21] | UPLC-MS (ES⁺, Long acidic): 2.82 min, m/z 471.6 [M + H]⁺ (100%) 1H NMR (400 MHz, DMSO-d₆): 8.52- 8.51 (m, 1H), 7.61 (s, 1H), 7.59-7.53 (m, 2H), 7.43-7.38 (m, 2H), 7.35-7.31 (m, 1H), 5.61-5.50 (m, 2H), 3.86-3.77 (m, 2H), 3.31 (s, 3H), 2.94 (s, 3H), 2.66 (s, 3H), 2.03-1.95 (m, 2H), 1.61-1.58 (m, 3H), 0.69-0.63 (m, 3H). |
| 197 | 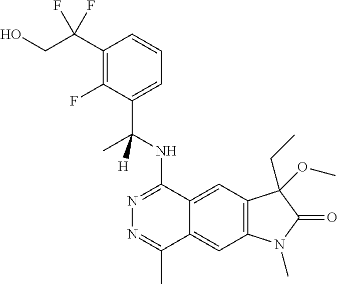 [SMILES: CCC1(OC)C(=O)N(C)c2cc3c(C)nnc(N[C@H](C)c4cccc(C(F)(F)CO)c4F)c3cc21] | UPLC-MS (ES⁺, Long acidic): 2.89 min, m/z 489.7 [M + H]⁺ (100%) 1H NMR (400 MHz, DMSO-d₆): 8.57- 8.55 (m, 1H), 7,61-7.54 (m, 2H), 7.42 (s, 1H), 7.41-7.35 (m, 1H), 7.21-7.18 (m, 1H), 5.75-5.66 (m, 2H), 3.99-3.91 (m, 2H), 2.97-2.94 (m, 3H), 2.66 (s, 3H), 2.05-1.95 (m, 2H), 1.61-1.58 (m, 3H), 0.72-0.64 (m, 3H). CH3 under solvent peak |
| 198 | 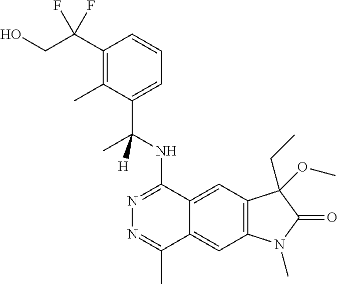 [SMILES: CCC1(OC)C(=O)N(C)c2cc3c(C)nnc(N[C@H](C)c4cccc(C(F)(F)CO)c4C)c3cc21] | UPLC-MS (ES⁺, Long acidic): 2.90 min, m/z 485.6 [M + H]⁺ (100%) 1H NMR (400 MHz, DMSO-d₆): 8.56- 8.55 (m, 1H), 7.64-7.57 (m, 2H), 7.40 (s, 1H), 7.34-7.7.29 (m, 1H), 7.24-7.18 (m, 1H), 5.75-5.65 (m, 2H), 3.96-3.86 (m, 2H), 3.31 (s, 3H), 2.95 (d, J = 3.9 Hz, 3H), 2.66-2.65 (m, 3H), 2.55-5.54 (m, 3H), 2.02-1.95 (m, 2H), 1.54-1.52 (m, 3H), 0.70-0.63 (m, 3H). |
| 199 | 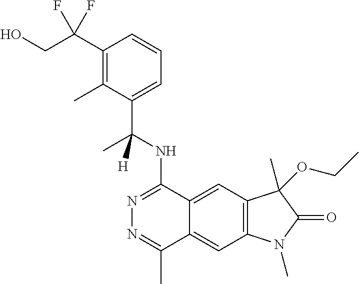 [SMILES: CCOC1(C)C(=O)N(C)c2cc3c(C)nnc(N[C@H](C)c4cccc(C(F)(F)CO)c4C)c3cc21] | UPLC-MS (ES⁺, Long acidic): 2.88 min, m/z 485.7 [M + H]⁺ (100%) 1H NMR (400 MHz, DMSO-d₆): 8.66- 8.55 (m, 1H), 7.66-7.51 (m, 2H), 7.40- 7.35 (m, 1H), 7.35-7.26 (m, 1H), 7.25- 7.16 (m, 1H), 5.78-5.61 (m, 2H), 3.99- 3.81 (m, 2H), 3.29 (s, 3H), 3.14-2.96 (m, 2H), 2.68-2.60- (m, 3H), 2.59-2.51 (m, 3H), 1.61-1.48 (m, 6H), 1.06 (t, J = 7.0 Hz, 3H) |
| 200 | 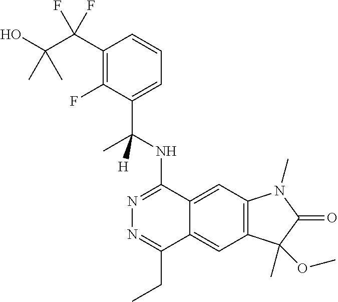 [SMILES: CCc1nnc(N[C@H](C)c2cccc(C(F)(F)C(C)(C)O)c2F)c2cc3c(cc12)C(C)(OC)C(=O)N3C] | UPLC-MS (ES⁺, Long acidic): 3.22 min, 517.7 m/z [M + H]⁺ (100%) 1H NMR (400 MHz, DMSO-d₆): 8.04 (s, 1H), 8.01 (s, 1H), 7.55 (m, 1H), 7.44 (m, 1H), 7.29 (m, 1H), 7.16 (m, 1H), 5.79-5.72 (m, 1H), 5.35 (s, 1H), 3.11- 3.04 (m, 2H) 2.92 (d, J = 3.16 Hz, 3H), 1.61 (d, J = 6.96 Hz, 3H), 1.25 (m, 9H). 6H under solvent peaks |
| 201 | 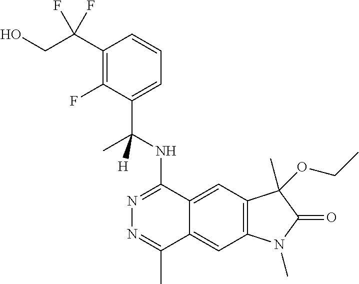 [SMILES: CCOC1(C)C(=O)N(C)c2cc3c(C)nnc(N[C@H](C)c4cccc(C(F)(F)CO)c4F)c3cc21] | UPLC-MS (ES⁺, Long acidic): 2.96 min, m/z 489.4 [M + H]⁺ (100%) 1H NMR (400 MHz, DMSO-d₆): 8.64- 8.55 (m, 1H), 7.64-7.49 (m, 2H), 7.45- 7.33 (m, 2H), 7.24-7.13 (m, 1H), 5.79- 5.66 (m, 2H), 4.03-3.87 (m, 2H), 3.30 (s, 3H), 3.14-2.95 (m, 2H), 2.65 (s, 3H), 1.65-1.47 (m, 6H), 1.11-1.03 (m, 3H) |
| 202 | 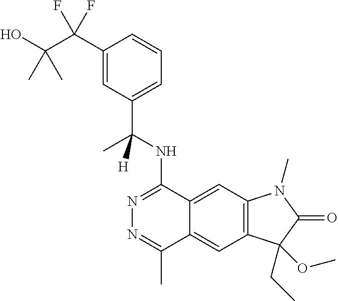 [SMILES: CCC1(OC)C(=O)N(C)c2cc3c(N[C@H](C)c4cccc(C(F)(F)C(C)(C)O)c4)nnc(C)c3cc21] | UPLC-MS (ES⁺, Long acidic): 4.86 min, m/z 499.8 [M + H]⁺ (98%) 1H NMR (400 MHz, DMSO-d₆): 8.01 (s, 1H), 7.90 (s, 1H), 7.58-7.49 (m, 2H), 7.45-7.39 (m, 1H), 7.39-7.33 (m, 1H), 7.33-7.27 (m, 1H), 5.56 (quintet, J = 7.0 Hz, 1H), 5.23-5.20 (m, 1H), 3.33 (s, 3H), 2.97-2.84 (m, 3H), 2.65 (s, 3H), 2.10-1.85 (m, 2H), 1.62 (d, J = 7.0 Hz, 3H), 1.12 (s, 3H), 1.09 (d, J = 5.7 Hz, 3H), 0.64 (t, J = 7.1 Hz, 3H) |
| 203 | 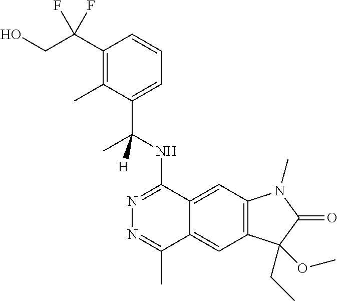 [SMILES: CCC1(OC)C(=O)N(C)c2cc3c(N[C@H](C)c4cccc(C(F)(F)CO)c4C)nnc(C)c3cc21] | UPLC-MS (ES⁺, Long acidic): 5.30 min, m/z 485.7 [M + H]⁺ (100%) 1H NMR (400 MHz, DMSO-d₆): 8.06- 8.00 (m, 1H), 7.89 (s, 1H), 7.66- 7.59 (m, 1H), 7.50-7.41 (m, 1H), 7.36- 7.29 (m, 1H), 7.25-7.16 (m, 1H), 5.81- 5.69 (m, 1H), 5.67 (t, J = 6.4 Hz, 1H), 4.01-3.79 (m, 2H), 3.34 (s, 3H), 2.96- 2.86 (m, 3H), 2.64 (s, 3H), 2.54 (s, 3H), 2.11-1.86 (m, 2H), 1.55 (d, J = 6.9 Hz, 3H), 0.64 (t, J = 7.0 Hz, 3H) |
| 204 | 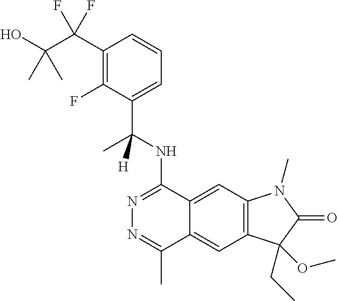 [SMILES: CCC1(OC)C(=O)N(C)c2cc3c(N[C@H](C)c4cccc(C(F)(F)C(C)(C)O)c4F)nnc(C)c3cc21] | UPLC-MS (ES⁺, Long acidic): 5.40 min, m/z 517.8 [M + H]⁺ (97%) 1H NMR (400 MHz, DMSO-d₆): 8.05 (s, 1H), 7.91 (s, 1H), 7.58-7.50 (m, 1H), 7.49-7.42 (m, 1H), 7.33-7.24 (m, 1H), 7.20-7.12 (m, 1H), 5.73 (quintet, J = 6.8 Hz, 1H), 5.34 (s, 1H), 3.35 (s, 3H), 2.93 (s, 3H), 2.65 (s, 3H), 2.09- 1.87 (m, 2H), 1.61 (d, J = 7.0 Hz, 3H), 1.24 (s, 3H), 1.23 (s, 3H), 0.69-0.57 (m, 3H) |
| 205 | 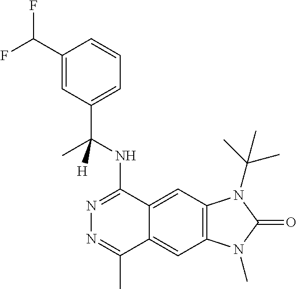 [SMILES: Cc1nnc(N[C@H](C)c2cccc(C(F)F)c2)c2cc3c(cc12)n(C)c(=O)n3C(C)(C)C] | UPLC-MS (ES⁺, Long acidic): 3.16 min, m/z 440.5 [M + H]⁺ (100%) 1H NMR (400 MHz, DMSO-d₆): 8.15 (s, 1H), 7.64-7.60 (m, 2H), 7.53 (s, 1H), 7.52-7.49 (m, 1H), 7.46-7.43 (m, 1H), 7.38-7.39 (m, 1H), 6.99 (t, J = 55.0 Hz, 1H), 5.66-5.55 (m, 1H), 3.41 (s, 3H), 2.64 (s, 3H), 1.85 (s, 9H), 1.63 (d, J = 7.1 Hz, 3H). |
| 206 | 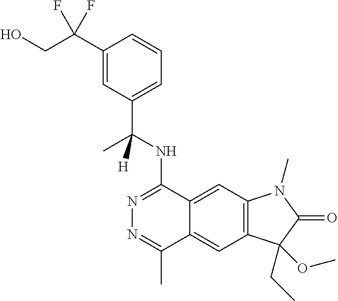 [SMILES: CCC1(OC)C(=O)N(C)c2cc3c(N[C@H](C)c4cccc(C(F)(F)CO)c4)nnc(C)c3cc21] | UPLC-MS (ES⁺, Long acidic): 2.72 min, m/z 471.5 [M + H]⁺ (100%) 1H NMR (400 MHz, DMSO-d₆): 8.01 (s, 1H), 7.90 (s, 1H), 7.61 (s, 1H), 7.59- 7.53 (m, 1H), 7.47-7.41 (m, 1H), 7.41- 7.37 (m, 1H), 7.37-7.30 (m, 1H), 5.65- 5.50 (m, 2H), 3.92-3.74 (m, 2H), 3.34 (s, 3H), 2.92 (s, 3H), 2.65 (s, 3H), 2.09- 1.87 (m, 2H), 1.61 (d, J = 7.0 Hz, 3H), 0.71-0.57 (m, 3H) |
| 207 | 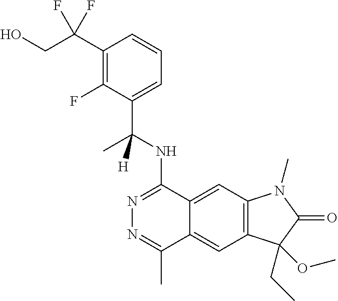 [SMILES: CCC1(OC)C(=O)N(C)c2cc3c(N[C@H](C)c4cccc(C(F)(F)CO)c4F)nnc(C)c3cc21] | UPLC-MS (ES⁺, Long acidic): 2.77 min, m/z 489.5 [M + H]⁺ (100%) 1H NMR (400 MHz, DMSO-d₆): 8.09- 8.0 (m, 1H), 7.91 (s, 1H), 7.62-7.52 (m, 1H), 7.51-7.43 (m, 1H), 7.43-7.34 (m, 1H), 7.25-7.15 (m, 1H), 5.81-5.66 (m, 2H), 4.04-3.88 (m, 2H), 3.36 (s, 3H), 2.93 (s, 3H), 2.65 (s, 3H), 2.11-1.84 (m, 2H), 1.61 (d, J = 7.0 Hz, 3H), 0.72-0.59 (m, 3H) |
| 208 | 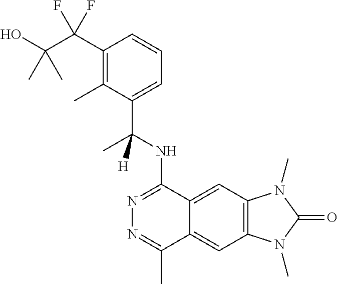 [SMILES: Cc1c([C@@H](C)Nc2nnc(C)c3cc4c(cc23)n(C)c(=O)n4C)cccc1C(F)(F)C(C)(C)O] | UPLC-MS (ES⁺, Long acidic): 2.89 min, m/z 470.7 [M + H]⁺ (100%) 1H NMR (400 MHz, DMSO-d₆): 8.19 (s, 1H), 7.62-7.60 (m, 1H), 7.57 (s, 1H), 7.29-7.27 (m, 1H), 7.24-7.22 (m, 1H), 7.18-7.14 (m, 1H), 5.81-5.77 (m, 1H), 5.30 (s, 1H), 3.49 (s, 3H), 3.46 (s, 3H), 2.65 (s, 3H), 2.59 (s, 3H), 1.54 (d, J - 7.0 Hz, 3H), 1.24 (s, 3H), 1.23 (s, 3H). |
| 209 | 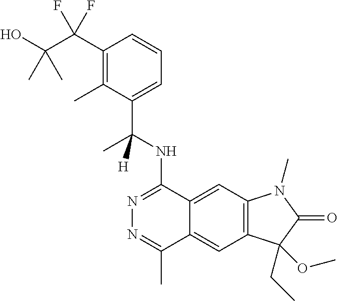 [SMILES: CCC1(OC)C(=O)N(C)c2cc3c(N[C@H](C)c4cccc(C(F)(F)C(C)(C)O)c4C)nnc(C)c3cc21] | UPLC-MS (ES⁺, Long acidic): 3.05 + 3.08 min, m/z 513.8 [M + H]⁺ (35 + 65%) 1H NMR (400 MHz, DMSO-d₆): 8.04 (s, 1H), 7.89 (s, 1H), 7.66-7.56 (m, 1H), 7.47-7.38 (m, 1H), 7.28-7.21 (m, 1H), 7.21-7.12 (m, 1H), 5.87-5.71 (m, 1H), 5.30 (s, 1H), 3.34 (s, 3H), 2.95- 2.87 (m, 3H), 2.65 (s, 3H), 2.59 (s, 3H), 2.08-1.85 (m, 2H), 1.55 (d, J = 6.9 Hz, 3H), 1.24 (s, 3H), 1.23 (s, 3H), 0.64 (t, J = 7.3 Hz, 3H) |
| 210 | 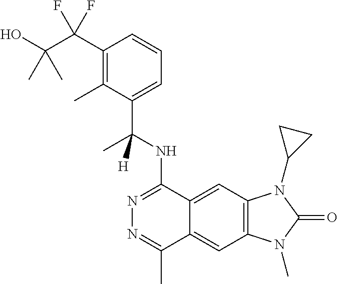 [SMILES: Cc1c([C@@H](C)Nc2nnc(C)c3cc4c(cc23)n(C2CC2)c(=O)n4C)cccc1C(F)(F)C(C)(C)O] | UPLC-MS (ES⁺, Long acidic): 3.03 min, m/z 496.6 [M + H]⁺ (100%) 1H NMR (400 MHz, DMSO-d₆): 8.17 (s, 1H), 7.64-7.62 (m, 1H), 7.54 (s, 1H), 7.46-7.44 (m, 1H), 7.24-7.22 (m, 1H), 7.19-7.14 (m, 1H), 5.81-5.77 (m, 1H), 5.30 (s, 1H), 3.42 (s, 3H), 3.08- 3.04 (m, 1H), 2.64 (s, 3H), 2.59 (s, 3H), 1.55 (d, J = 6.9 Hz, 3H), 1.25 (s, 3H), 1.23 (s, 3H), 1.17-1.16 (m, 2H), 1.07- 0.97 (m, 2H). |
| 211 | 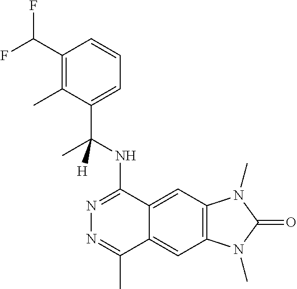 [SMILES: Cc1c(C(F)F)cccc1[C@@H](C)Nc1nnc(C)c2cc3c(cc12)n(C)c(=O)n3C] | UPLC-MS (ES⁺, Long acidic): 2.91 min, m/z 412.4 [M + H]⁺ (100%) 1H NMR (400 MHz, DMSO-d₆) 8.18 (s, 1H), 7.64 (m, 1H), 7.59 (s, 1H), 7.37-7.07 (m, 4H), 5.72 (m, 1H), 3.50 (s, 3H), 3.47 (s, 3H), 2.64 (s, 3H), 1.55 (d, J = 6.92 Hz, 3H), CH₃ under solvent peak |
| 212 | 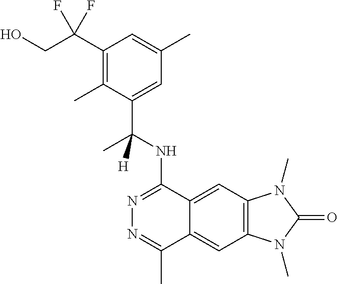 [SMILES: Cc1cc([C@@H](C)Nc2nnc(C)c3cc4c(cc23)n(C)c(=O)n4C)c(C)c(C(F)(F)CO)c1] | UPLC-MS (ES⁺, Long acidic): 2.74 min, m/z 456.4 [M + H]⁺ (100%) 1H NMR (400 MHz, DMSO-d₆) 8.18 (s, 1H), 7.59 (s, 1H), 7.44 (s, 1H), 7.26 (m, 1H), 7.13 (s, 1H), 5.70 (m, 1H), 5.64 (t, J = 6.16 Hz, 1H), 3.94-3.83 (m, 2H), 3.49 (s, 3H), 3.47 (s, 3H), 2.66 (s, 3H), 2.22 (s, 3H), 1.53 (d, J = 6.92 Hz, 3H). |
| 213 | 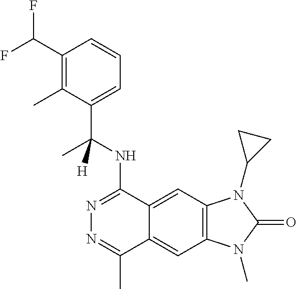 [SMILES: Cc1c(C(F)F)cccc1[C@@H](C)Nc1nnc(C)c2cc3c(cc12)n(C1CC1)c(=O)n3C] | UPLC-MS (ES⁺, Long acidic): 3.02 min, m/z 438.6 [M + H]⁺ (100%) 1H NMR (400 MHz, DMSO-d₆): 8.17 (s, 1H), 7.68-7.75 (m, 1H), 7.56 (s, 1H), 7.52-7.49 (s, 1H), 7.73-7.33 (m, 1H) 7.26-7.21 (m, 1H), 7.22 (t, J = 55.6 Hz, 1H), 5.74-5.68 (m, 1H), 4.42 (s, 3H), 3.09-3.03 (m, 1H), 2.64 (s, 3H), 2.52 (s, 3H), 1.56 (d, J = 7.0 Hz, 3H), 1.19-1.16 (m, 2H), 1.09-0.97 (m, 2H). |
| 214 | 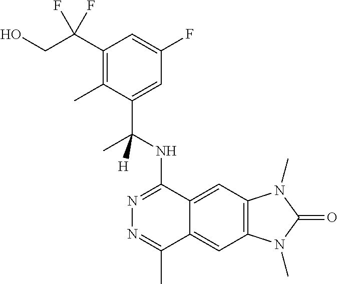 [SMILES: Cc1c([C@@H](C)Nc2nnc(C)c3cc4c(cc23)n(C)c(=O)n4C)cc(F)cc1C(F)(F)CO] | UPLC-MS (ES⁺, Long acidic): 2.71 min, m/z 460.3 [M + H]⁺ (100%) 1H NMR (400 MHz, DMSO-d₆) 8.16 (s, 1H), 7.61 (s, 1H), 7.43 (m, 1H), 7.35 (m, 1H), 7.09 (m, 1H), 5.75-5.69 (m, 2H), 3.98-3.89 (m, 2H), 3.51 (s, 3H), 3.48 (s, 3H), 2.66 (s, 3H), 1.56 (d, J = 7.00 Hz, 3H). CH₃ under solvent peak |
| 215 | 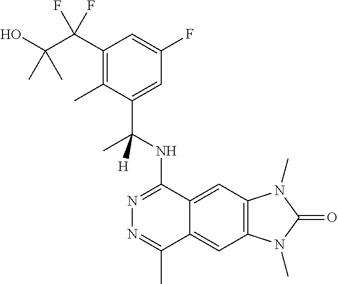 [SMILES: Cc1c([C@@H](C)Nc2nnc(C)c3cc4c(cc23)n(C)c(=O)n4C)cc(F)cc1C(F)(F)C(C)(C)O] | UPLC-MS (ES⁺, Long acidic): 3.01 min, m/z 488.4 [M + H]⁺ (100%) 1H NMR (400 MHz, DMSO-d₆) 8.16 (s, 1H), 7.60 (s, 1H), 7.42 (m, 1H), 7.32 (m, 1H), 7.00 (m, 1H), 5.77-5.70 (m, 1H), 5.40 (s, 1H), 3.51 (s, 3H), 3.47 (s, 3H), 2.66 (s, 3H), 2.56 (s, 3H), 1.54 (d, J = 6.88 Hz, 3H), 1.26-1.22 (m, 6H) |
| 216 | 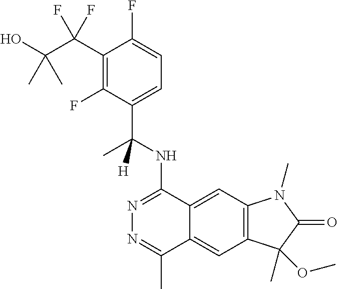 [SMILES: COC1(C)C(=O)N(C)c2cc3c(N[C@H](C)c4ccc(F)c(C(F)(F)C(C)(C)O)c4F)nnc(C)c3cc21] | UPLC-MS (ES⁺, Long acidic): 2.87 min, m/z 521.7 [M + H]⁺ (100%) 1H NMR (400 MHz, DMSO-d₆): 8.02 (s, 1H), 7.98 (s, 1H), 7.58-7.51 (m, 1H), 7.49-7.46 (m, 1H), 7.09-7.04 (m, 1H), 5.69-5.65 (m, 1H), 5.45 (s, 1H), 3.34 (s, 3H), 2.92 (d, J = 3.4 Hz, 3H), 2.65 (s, 3H), 1.60 (d, J - 7.3 Hz, 3H), 1.54 (s, 3H), 1.25 (s, 6H). |

### 1,8-dimethyl-3-tetrahydrofuran-3-yl-5-[[(1R)-1-[3-(trifluoromethyl)phenyl]ethyl]amino]imidazo[4,5-g]phthalazin-2-one (144)

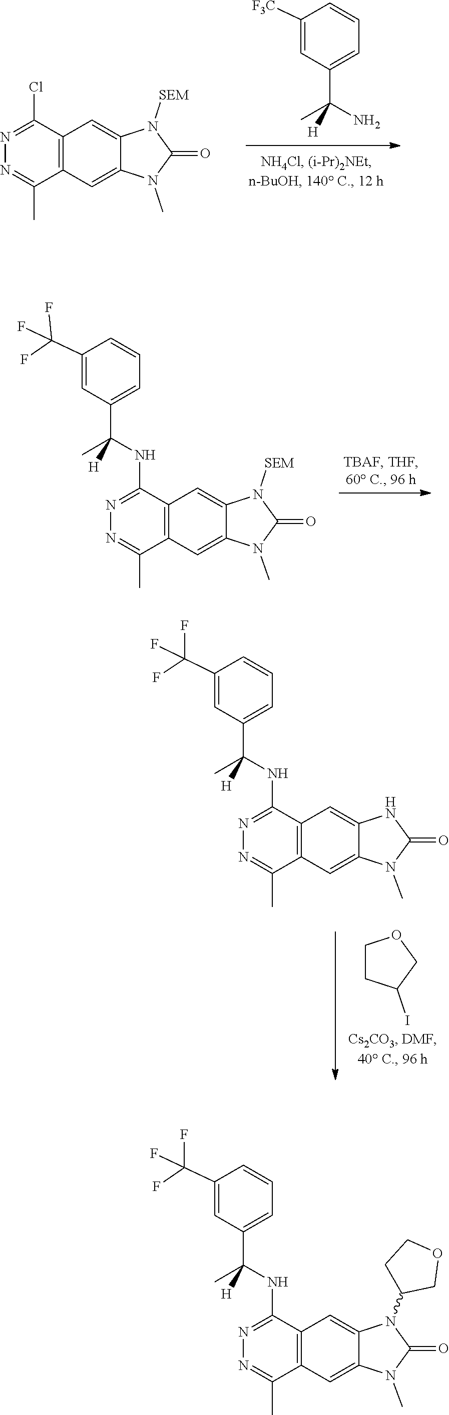

### Step 1

A solution of 5-chloro-1,8-dimethyl-3-(2-trimethylsilylethoxymethyl)imidazo[4,5-g]phthalazin-2-one (507 mg, 1.34 mmol), N,N-diisopropylethylamine (350 μL, 2.01 mmol), (1R)-1-[3-(trifluoromethyl)phenyl]ethylamine (230 μL, 1.34 mmol) and ammonium chloride (107 mg, 2.01 mmol) in 1-Butanol (14 mL) was placed in a sealed vial and heated to 140° C. overnight.

Reaction mixture was cooled to RT and concentrated. The crude product was purified by flash column chromatography (0-100% EtOAc in petroleum ether, then 0-20% MeOH in DCM). The mixture was then purified by reverse phase flash chromatography (eluting in 0-100% acetonitrile+0.1% formic acid and water+0.1% formic acid). Like fractions were pooled and loaded onto an SCX cartridge eluting with 1M NH₃ in MeOH to yield 1,8-dimethyl-5-[[(1R)-1-[3-(trifluoromethyl)phenyl]ethyl]amino]-3-(2-trimethylsilylethoxymethyl)imidazo[4,5-g]phthalazin-2-one (162 mg, 0.30 mmol, 22.8% yield) as a yellow oil.

UPLC-MS (ES⁺, Short acidic): 1.87 min, m/z 532.2 [M+H]⁺ (100%)

¹H NMR (400 MHz, CDCl₃) δ 7.75-7.71 (m, 2H), 7.52-7.48 (m, 2H), 7.47-7.41 (m, 1H), 7.37 (s, 1H), 5.76-5.69 (m, 1H), 5.48 (s, 2H), 5.18 (d, J=5.4 Hz, 1H), 3.69 (t, J=8.2 Hz, 2H), 3.59 (s, 3H), 2.85 (s, 3H), 1.73 (d, J=6.9 Hz, 3H), 1.06-0.95 (m, 2H) −0.00 (s, 9H).

### Step 2

To a solution of 1,8-dimethyl-5-[[(1R)-1-[3-(trifluoromethyl)phenyl]ethyl]amino]-3-(2-trimethylsilylethoxymethyl)imidazo[4,5-g]phthalazin-2-one (162.mg, 0.3 mmol) in THF (4 mL) was added tetrabutylammonium fluoride 1.0 M in THF (1.52 mL, 1.52 mmol). The reaction was heated at 60° C. for 96 h. The reaction was quenched with sat aq. solution NH₄Cl and diluted with EtOAc, the two layers were separated and the aqueous layer was extracted with EtOAc (×3).

Combined organic extracts were dried over Na₂SO₄, filtered and concentrated. The crude material was purified by reverse phase column chromatography (eluting in 0-50% acetonitrile+0.1% formic acid in water+0.1% formic acid) like fractions were pooled and loaded onto an SCX cartridge eluting with 1M NH₃ in MeOH to yield 1,8-dimethyl-5-[[(1R)-1-[3-(trifluoromethyl)-phenyl]ethyl]amino]-3H-imidazo[4,5-g]phthalazin-2-one (61 mg, 0.15 mmol, 49.9% yield) as a pale yellow solid.

UPLC-MS (ES⁺, Short acidic): 1.44 min, m/z 402.1 [M+H]⁺ (100%)

¹H-NMR (400 MHz, DMSO-d₆): δ 11.70 (s, 1H), 8.01 (s, 1H), 7.78 (s, 1H), 7.76-7.72 (m, 1H), 7.55-7.51 (m, 3H), 7.44 (d, J=7.5 Hz, 1H), 5.58-5.48 (m, 1H), 3.44 (s, 3H), 2.65 (s, 3H), 1.59 (d, J=7.0 Hz, 3H).

### Step 3

To a solution of 1,8-dimethyl-5-[[(1R)-1-[3-(trifluoromethyl)phenyl]ethyl]amino]-3H-imidazo[4,5-g]phthalazin-2-one (61 mg, 0.15 mmol) and cesium carbonate (74 mg, 0.23 mmol) in DMF (0.6 mL) was added 3-iodotetrahydrofuran (0.02 mL, 0.15 mmol) and the reaction stirred at rt for 2 days. Cesium carbonate (49.5 mg, 0.15 mmol) and 3-iodotetrahydrofuran (0.01 mL, 0.08 mmol) were added and the reaction stirred at rt for 1 day. The reaction was heated to 40° C. for 6 h. Cesium carbonate (74 mg, 0.23 mmol) and 3-iodotetrahydrofuran (0.02 mL, 0.15 mmol) were added and the reaction stirred at 40° C. overnight. The reaction mixture was cooled to RT and concentrated The crude material was purified by flash column chromatography (eluting in 0-100% EtOAc in petroleum ether then 0-8% MeOH in DCM) to yield 1,8-dimethyl-3-tetrahydrofuran-3-yl-5-[[(1R)-1-[3-(trifluoromethyl)phenyl]ethyl]amino]imidazo[4,5-g]phthalazin-2-one (26.5 mg, 0.0562 mmol, 36.985% yield) as a white solid.

UPLC-MS (ES⁺, Long acidic): 3.16 min, m/z 472.5 [M+H]⁺ (100%).

1H-NMR (400 MHz, DMSO-d₆): 8.20-8.17 (m, 1H), 7.81-7.73 (m, 2H), 7.61 (s, 1H), 7.57-7.51 (m, 2H), 7.24-7.21 (m, 1H), 5.61-5.51 (m, 1H), 5.16-5.03 (m, 1H), 4.22-4.14 (m, 1H), 4.12-4.07 (m, 1H), 4.05-3.98 (m, 1H), 3.96-3.89 (, 1H), 3.46 (s, 3H), 2.66 (s, 3H), 2.41-2.35 (i, 2H), 1.63 (d, J (d7.1 Hz, 3H).

Compounds 146-149 were prepared in a similar manner, starting from the corresponding chlorophthalazine and respective amine

TABLE 4B
Compounds Prepared According to the Disclosed Methods

| Compound | Structure | Analytical data |
|---|---|---|
| 145 | 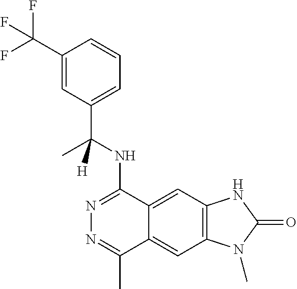 [SMILES: Cc1nnc(N[C@H](C)c2cccc(C(F)(F)F)c2)c2cc3[nH]c(=O)n(C)c3cc12] | UPLC-MS (ES⁺, long acidic): 2.95 min, m/z 402.4 [M + H]⁺ (100%) ¹H-NMR (400 MHz, DMSO-d₆): 11.70 (s, 1H), 8.01 (s, 1H), 7.78 (s, 1H), 7.76-7.72 (m, 1H), 7.55-7.51 (m, 3H), 7.44 (d, J = 7.5 Hz, 1H), 5.58- 5.48 (m, 1H), 3.44 (s, 3H), 2.65 (s, 3H), 1.59 (d, J = 7.0 Hz, 3H). |
| 146 | 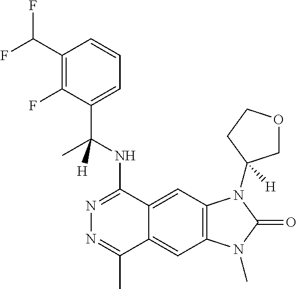 [SMILES: Cc1nnc(N[C@H](C)c2cccc(C(F)F)c2F)c2cc3c(cc12)n(C)c(=O)n3[C@H]1CCOC1] | UPLC-MS (ES⁺, Long acidic): 3.07 min, m/z 472.4 [M + H]⁺ (98%). 1H-NMR (400 MHz, DMSO-d₆): 8.23 (s, 1H), 7.65-7.59 (m, 2H), 7.49-7.44 (m, 1H), 7.26 (t, J = 54.5 Hz, 1H), 7.26-7.20 (m, 2H), 5.74-5.67 (m, 1H), 5.18-5.08 (m, 1H), 4.23-4.16 (m, 1H), 4.13- 4.08 (m, 1H), 4.06-4.00 (m, 1H), 3.98-3.91 (m, 1H), 3.47 (s, 3H), 2.66 (s, 3H), 2.43-2.35 (m, 2H,), 1.63 (d, J = 7.1 Hz, 3H). |
| 147 | 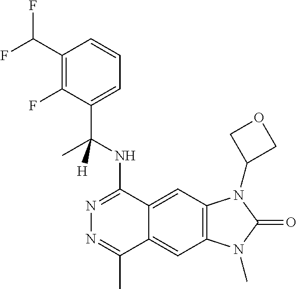 [SMILES: Cc1nnc(N[C@H](C)c2cccc(C(F)F)c2F)c2cc3c(cc12)n(C)c(=O)n3C1COC1] | UPLC-MS (ES⁺, Long acidic2.98 min, m/z 458.1 [M + H]⁺ (93%). 1H-NMR (400 MHz, DMSO-d₆): 8.25 (s, 1H), 7.69-7.58 (m, 2H), 7.48-7.44 (m, 1H), 7.43-7.34 (m, 1H), 7.26 (t, J = 54.6 Hz, 1H), 7.26-7.21 (m, 1H), 5.74-5.63 (m, 2H), 5.40-5.32 (m, 2H), 4.98- 4.88 (m, 2H), 3.49 (s, 3H), 2.67 (s, 3H), 1.64 (d, J = 7.0 Hz, 3H). |
| 148 | 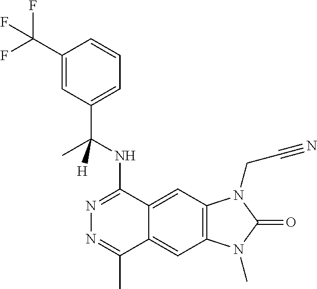 [SMILES: Cc1nnc(N[C@H](C)c2cccc(C(F)(F)F)c2)c2cc3c(cc12)n(C)c(=O)n3CC#N] | UPLC-MS (ES⁺, Long acidic2.97 min, m/z 441.4 [M + H]⁺ (96%) ¹H NMR (400 MHz, DMSO-d₆) 8.33 (s, 1H), 7.80-7.74 (m, 2H), 7.71 (s, 1H), 7.58-7.53 (m, 2H), 7.32 (d, J = 7.1 Hz, 1H), 5.61-5.52 (m, 1H), 5.23-5.11 (m, 2H), 3.51 (s, 3H), 2.68 (s, 3H), 1.65 (d, J = 7.1 Hz, 3H). |
| 149 | 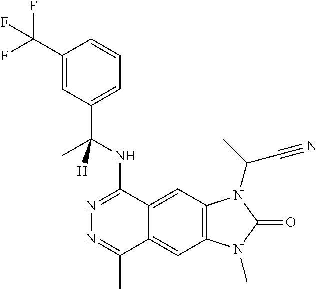 [SMILES: Cc1nnc(N[C@H](C)c2cccc(C(F)(F)F)c2)c2cc3c(cc12)n(C)c(=O)n3C(C)C#N] | UPLC-MS (ES⁺, Long acidic): 3.05 min, m/z 455.6 [M + H]⁺ (100%) ¹H NMR (400 MHz, DMSO-d₆) 8.27 (d, J = 7.0 Hz, 1H), 7.80-7.72 (m, 2H), 7.70 (d, J = 2.0 Hz, 1H), 7.58-7.53 (m, 2H), 7.47-7.40 (m, 1H), 5.91-5.84 (m, 1H), 5.64-5.55 (m, 1H), 3.50- 3.49 (m, 3H), 2.68-2.66 (m, 3H), 1.92 (d, J = 7.3 Hz, 3H), 1.67-1.63 (m, 3H) |

### 1,3,8-trimethyl-5-[[rac-(1R)-1-[2-methyl-3-(2-methylpyrazol-3-yl)phenyl]ethyl]amino]imidazo[4,5-g]phthalazin-2-one (150)

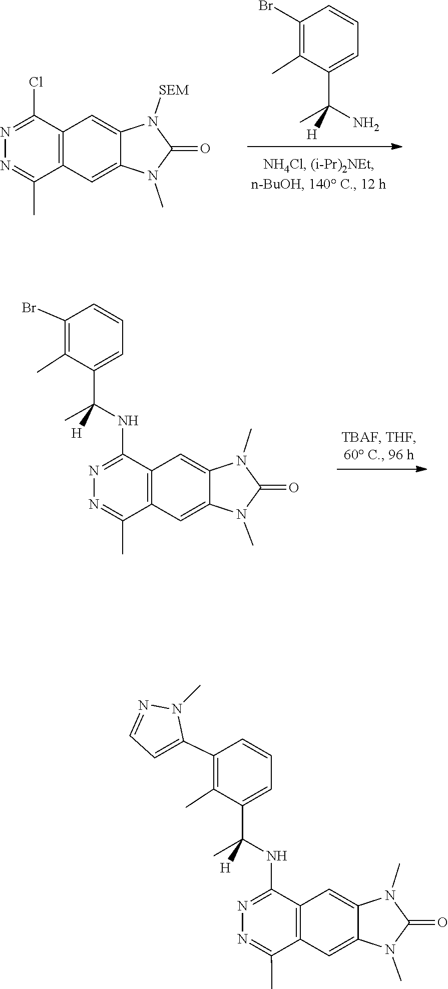 [SMILES: Cc1nnc(Cl)c2cc3c(cc12)n(C)c(=O)n3COCC[Si](C)(C)C ; Cc1c(Br)cccc1[C@@H](C)N ; Cc1c(Br)cccc1[C@@H](C)Nc1nnc(C)c2cc3c(cc12)n(C)c(=O)n3C ; Cc1c(-c2ccnn2C)cccc1[C@@H](C)Nc1nnc(C)c2cc3c(cc12)n(C)c(=O)n3C]

### Step 1

To a vial was added, 5-chloro-1,3,8-trimethyl-imidazo[4,5-g]phthalazin-2-one (369 mg, 1.4 mmol) and (1R)-1-(3-bromo-2-methyl-phenyl)ethanamine (241 mg, 1.12 mmol) in 1-butanol (4.5 mL). N,N-diisopropylethylamine (1.22 mL, 7.02 mmol) and ammonium chloride (225.mg, 4.21 mmol) were added. The vial was sealed and the reaction was heated to 140° C. After 24 hrs, N,N-diisopropylethylamine (1.22 mL, 7.02 mmol) was added and RM mixed at 140° C. over-the-weekend. The reaction mixture was cooled to RT and concentrated in vacuo. The residue purified by flash chromatography (25 g, eluting in 0-20% MeOH in DCM). Like fractions were pooled and concentrated in vacuo. The residue was passed through an SCX cartridge (2 g, pre-equilibrated with MeOH flushing with 1.0M NH₃ in MeOH) to give 5-[[(1R)-1-(3-bromo-2-methyl-phenyl)ethyl]amino]-1,3,8-trimethyl-imidazo[4,5-g]phthalazin-2-one (142 mg, 0.32 mmol, 23.0% yield) as an orange oil.

UPLC-MS (ES⁺, Short acidic): 1.43 min, m/z 442.1 [M+H]⁺ (77%)

### Step 2

A suspension of 1,3,8-trimethyl-5-[[rac-(1R)-1-(3-bromo-2-methyl-phenyl)ethyl]⁻ amino]imidazo[4,5-g]phthalazin-2-one (75.mg, 0.17 mmol), 1-methyl-1H-pyrazole-5-boronic acid, pinacolester (46.mg, 0.22 mmol) and Potassium carbonate (71 mg, 0.51 mmol) in 1,4-dioxane (1.2 mL) and water (0.3 mL) was degassed for 5 mins. [1,1′-bis(diphenylphosphino)ferrocene]Palladium(II) chloride dichloromethane complex (14 mg, 0.02 mmol) was added, and the reaction was stirred at 100° C. for 16 hours. The reaction mixture was cooled to RT, filtered through hydrophobic filter paper, washing with EtOAc. The filtrate concentrated in vacuo. The residue was purified by flash chromatography (4 g, 0-20% MeOH in DCM) like fractions were pooled and concentrated in vacuo to yield 1,3,8-trimethyl-5-[[(1R)-1-[2-methyl-3-(2-methylpyrazol-3-yl)phenyl]ethyl]amino]imidazo[4,5-g]phthalazin-2-one (29.5 mg, 0.067 mmol, 39.2% yield) as a brown solid.

UPLC-MS (ES⁺, Long acidic): 2.76 min, m/z 442.4 [M+H]⁺ (95%)

1H NMR (400 MHz, DMSO-d₆): δ 8.21 (s, 1H), 7.62-7.60 (m, 1H), 7.58 (s, 1H), 7.50-7.49 (m, 1H), 7.34-7.32 (m, 1H), 7.24-7.19 (m, 1H), 7.08-7.06 (m, 1H), 6.24 (d, J=1.9 Hz, 1H), 5.76-5.66 (m, 1H), 3.57 (s, 3H), 3.51 (s, 3H), 3.47 (s, 3H), 2.65 (s, 3H), 2.26 (s, 3H), 1.59 (d, J=7.9 Hz, 3H).

The following compounds were prepared in a similar manner, starting from the corresponding chlorophthalazine and respective amine

TABLE 4C
Compounds Prepared According to the Disclosed Methods

| Compound | Structure | Analytical data |
|---|---|---|
| 151 | 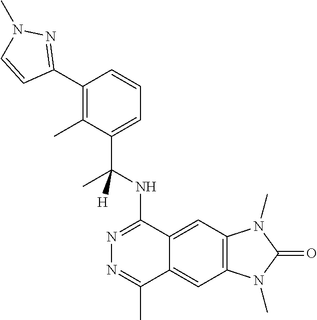 [SMILES: Cc1c(-c2ccn(C)n2)cccc1[C@@H](C)Nc1nnc(C)c2cc3c(cc12)n(C)c(=O)n3C] | UPLC-MS (ES⁺, Long acidic): 2.76 min, m/z 442.3 [M + H]⁺ (100%) 1H NMR (400 MHz, DMSO-d₆): 8.20 (s, 1H), 7.63-7.59 (m, 1H), 7.57 (s, 1H), 7.50 (d, J = 1.7 Hz, 1H), 7.35-7.29 (m, 1H), 7.25-7.18 (m, 1H), 7.09-7.03 (m, 1H), 6.24 (d, J = 1.7 Hz, 1H), 5.71 (quint, J = 7.0 Hz, 1H), 3.58 (s, 3H), 3.50 (s, 3H), 3.47 (s, 3H), 2.65 (s, 3H), 2.26 (s, 3H), 1.59 (d, J = 6.9 Hz, 3H) |
| 152 | 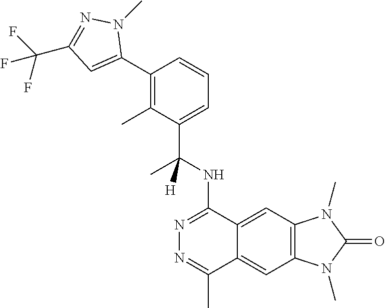 [SMILES: Cc1c(-c2cc(C(F)(F)F)nn2C)cccc1[C@@H](C)Nc1nnc(C)c2cc3c(cc12)n(C)c(=O)n3C] | UPLC-MS (ES⁺, Long acidic): 3.24 min, m/z 510.5 [M + H]⁺ (100%) 1H NMR (400 MHz, DMSO-d₆) 8.21(s, 1H), 7.67-7.64(m, 1H), 7.59(s, 1H), 7.36-7.34(m, 1H), 7.28-7.24(m, 1H), 7.16-7.14(m, 1H), 6.78(s, 1H), 5.75-5.69(m, 1H), 3.67(s, 3H), 3.51(s, 3H), 3,47(s, 3H), 2.65(s, 3H), 2.28(s, 3H), 1.59(d, J =7.0 Hz, 3H). |
| 153 | 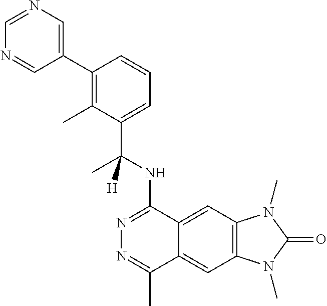 [SMILES: Cc1c(-c2cncnc2)cccc1[C@@H](C)Nc1nnc(C)c2cc3c(cc12)n(C)c(=O)n3C] | UPLC-MS (ES⁺, Long acidic): 2.57 min, m/z 440.4 [M + H]⁺ (100%) 1H NMR (400 MHz, DMSO-d₆): 9.21 (s, 1H), 8.83 (s, 2H), 8.22 (s, 1H), 7.64-7.60 (m, 1H), 7.59 (s, 1H), 7.37-7.33 (m, 1H), 7.29-7.21 (m, 1H), 7.17-7.10 (m, 1H), 5.75 (quint, J = 7.1 Hz, 1H), 3.51 (s, 3H), 3.47 (s, 3H), 2.65 (s, 3H), 2.38 (s, 3H), 1.60 (d, J = 6.9 Hz, 3H) |
| 154 | 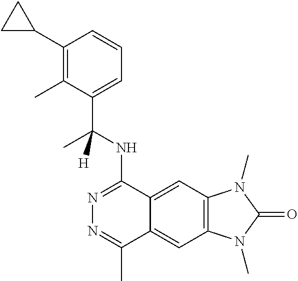 [SMILES: Cc1c(C2CC2)cccc1[C@@H](C)Nc1nnc(C)c2cc3c(cc12)n(C)c(=O)n3C] | UPLC-MS (ES⁺, Long acidic): 3.11 min, m/z 402.4 [M + H]⁺ (100%) 1H NMR (400 MHz, DMSO-d₆): 8.20 (s, 1H), 7.57 (s, 1H), 7.34-7.29 (m, 1H), 7.25-7.17 (m, 1H), 7.05-6.95 (m, 1H), 6.89-6.80 (m, 1H), 5.75 (quint, J = 7.1 Hz, 1H), 3.48 (s, 3H), 3.46 (s, 3H), 2.65 (s, 3H), 1.99-1.84 (m, 1H), 1.53 (d, J = 6.9 Hz, 3H), 0.96-0.85 (m, 2H), 0.62- 0.49 (m, 2H), signal for CH3 under DMSO peak |
| 155 | 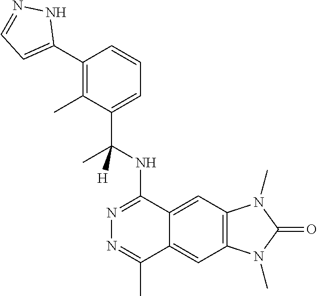 [SMILES: Cc1c(-c2ccn[nH]2)cccc1[C@@H](C)Nc1nnc(C)c2cc3c(cc12)n(C)c(=O)n3C] | UPLC-MS (ES⁺, Long acidic): 2.67 min, m/z 428.2 [M + H]⁺ (98%) 1H NMR (400 MHz, DMSO-d₆): 8.22 (s, 1H), 7.78 (br s, 1H), 7.58 (s, 1H), 7.50 (br s, 1H), 7.36-7.27 (m, 1H), 7.27-7.05 (m, 2H), 6.38 (s, 1H), 5.74 (quint, J = 7.2 Hz, 1H), 3.50 (s, 3H), 3.47 (s, 3H), 2.65 (s, 3H), 1.58 (d, J = 6.9 Hz, 3H), CH₃ under solvent peak |
| 156 | 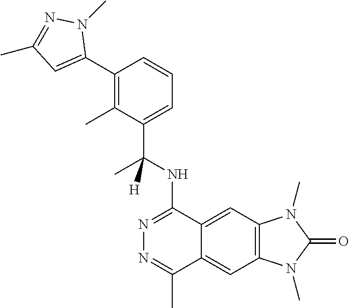 [SMILES: Cc1cc(-c2cccc([C@@H](C)Nc3nnc(C)c4cc5c(cc34)n(C)c(=O)n5C)c2C)n(C)n1] | UPLC-MS (ES⁺, Long acidic): 2.84 min, m/z 456.3 [M + H]⁺ (100%) 1H NMR (400 MHz, DMSO-d₆): 8.20 (s, 1H), 7.65-7.50 (m, 2H), 7.38-7.28 (m, 1H), 7.25-7.13 (m, 1H), 7.08-6.99 (m, 1H),6.01 (s, 1H), 5.70 (quint, J = 7.0 Hz, 1H), 3.50 (s, 3H), 3.48 (s, 3H), 3.46 (s, 3H), 2.65 (s, 3H), 2.26 (s, 3H), 2.19 (s, 3H), 1.58 (d, J = 6.9 Hz, 3H) |
| 157 | 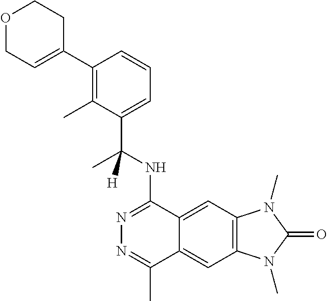 [SMILES: Cc1c(C2=CCOCC2)cccc1[C@@H](C)Nc1nnc(C)c2cc3c(cc12)n(C)c(=O)n3C] | UPLC-MS (ES⁺, Long acidic): 2.90 min, m/z 444.3 [M + H]⁺ (92%) 1H NMR (400 MHz, DMSO-d₆): 8.20(s, 1H), 7.58(s, 1H), 7.43-7.40(m, 1H), 7.23(d, J = 7.4 Hz, 1H), 7.09-7.05(m, 1H), 6.92-6.89(m, 1H), 5.72-5.68(m, 1H), 5.59-5.57(m, 1H), 4.21- 4.17(m, 2H), 3.83(t, J = 5.5 Hz, 2H), 3.49(s, 3H), 3.47(s, 3H), 2.65(s, 3H), 2.39(s, 3H), 2.27- 2.16(, 2H), 1.53(d, J = 7.0 Hz, 3H). |
| 158 | 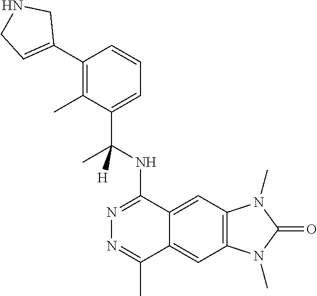 [SMILES: Cc1c(C2=CCNC2)cccc1[C@@H](C)Nc1nnc(C)c2cc3c(cc12)n(C)c(=O)n3C] | UPLC-MS (ES⁺, Long acidic): 2.06 min, m/z 429.3 [M + H]⁺ (100%) 1H NMR (400 MHz, DMSO-d₆): 8.21 (s, 1H), 7.59 (s, 1H), 7.45-7.40 (m, 1H), 7.30-7.23 (m, 1H), 7.11-7.05 (m, 1H), 7.01-6.96 (m, 1H), 5.84-5.79 (m, 1H), 5.77-5.68 (m, 1H), 3.97-3.78 (m, 4H), 3.51 (s, 3H), 3.48 (s, 3H), 2.66 (s, 3H), 2.44 (s, 3H), 1.56 (d, J = 6.9 Hz, 3H). NH not observed |
| 159 | 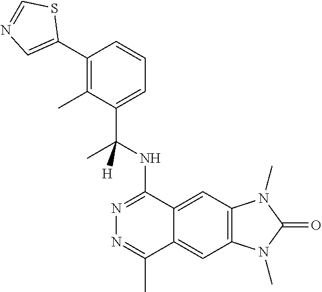 [SMILES: Cc1c(-c2cncs2)cccc1[C@@H](C)Nc1nnc(C)c2cc3c(cc12)n(C)c(=O)n3C] | UPLC-MS (ES⁺, Long acidic): 2.81 min, m/z 445.3 [M + H]⁺ (100%) 1H NMR (400 MHz, Methanol-d4) δ 9.07 (s, 1H), 8.54 (s, 1H), 8.23 (s, 1H), 7.81 (s, 1H), 7.73 (s, 1H), 7.62-7.58 (m, 1H), 7.27 - 7.15 (m, 2H), 5.77-5.69 (m,1H), 3.63 (s, 3H), 3.59 (s, 3H), 2.79 (s, 3H), 2.51 (s, 3H), 1.71 (d, J = 6.9 Hz, 3H), |

### 1,3,8-trimethyl-5-[[(1R)-1-(2-methyl-3-tetrahydropyran-4-yl-phenyl)ethyl]amino]imidazo[4,5-g]phthalazin-2-one (160)

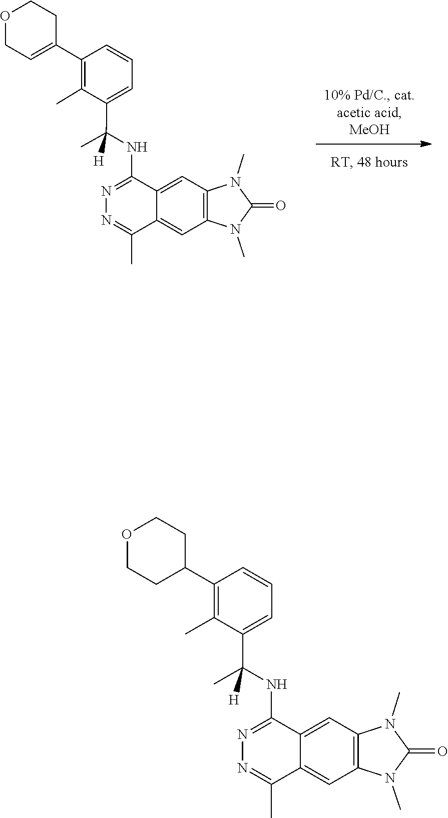 [SMILES: Cc1c(C2=CCOCC2)cccc1[C@@H](C)Nc1nnc(C)c2cc3c(cc12)n(C)c(=O)n3C ; Cc1c(C2CCOCC2)cccc1[C@@H](C)Nc1nnc(C)c2cc3c(cc12)n(C)c(=O)n3C]

To a suspension of 5-[[I(1R)-1-[3-(3,6-dihydro-2H-pyran-4-yl)-2-methyl-phenyl]-ethyl]amino]-1,3,8-trimethyl-imidazo[4,5-g]phthalazin-2-one (49.mg, 0.11 mmol) in methanol (3 mL) was added Palladium, 10 wt. % on carbon powder, dry (117.54 mg, 0.11 mmol) and of Acetic Acid (Glacial) (16.07 uL, 0.28 mmol). Flask is evacuated and re-filled with N₂×3. Then placed under a H₂ atmosphere via 3× vac/H2 cycles. Stirred for 2 days at RT. Upon completion, mixture filtered through a pad of celite, rinsing thoroughly with MeOH. Filtrate concentrated in vacuo. Crude residue purified by flash chromatography (4 g, dry-load, 4-8% MeOH in DCM). Relevant fractions concentrated, and the residue passed through an SCX cartridge (1 g, pre-equilibrated with MeOH flushing with 1.0M NH₃ in MeOH). The ammonia filtrate concentrated in vacuo to give 1,3,8-trimethyl-5-[[(1R)-1-(2-methyl-3-tetrahydropyran-4-yl-phenyl)-ethyl]amino]imidazo[4,5-g]phthalazin-2-one (18.4 mg, 0.0413 mmol, 37.4% yield) as an off-white solid.

UPLC-MS (ES⁺, Long acidic): 2.84 min, m/z 446.3 [M+H]⁺ (93%)

1H NMR (400 MHz, DMSO-d₆): δ 8.22 (s, 1H), 7.59 (s, 1H), 7.37-7.32 (m, 1H), 7.28-7.22 (m, 1H) 7.09-7.07 (m, 2H), 5.78-5.72 (m, 1H), 3.99-3.91 (m, 2H), 3.54-3.50 (m, 2H), 3.49 (s, 3H), 3.47 (s, 3H), 3.11-3.05 (m, 1H), 2.66 (s, 3H), 2.42 (s, 3H), 1.69-1.59 (m, 4H), 1.53 (d, J=6.9 Hz, 3H).

Compound 161 was prepared in a similar manner, starting from the corresponding chlorophthalazine and respective amine

TABLE 4D
Compounds Prepared According to the Disclosed Methods

| Compound | Structure | Analytical data |
|---|---|---|
| 161 | 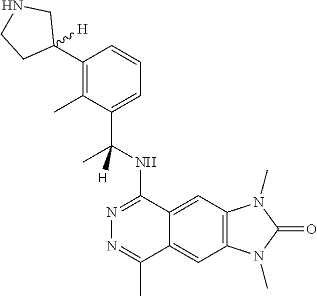 [SMILES: Cc1c(C2CCNC2)cccc1[C@@H](C)Nc1nnc(C)c2cc3c(cc12)n(C)c(=O)n3C] | UPLC-MS (ES⁺, Long acidic): 2.03 min + 2.07 min m/z 431.4 [M + H]⁺ (48 + 52%) 1H NMR (400 MHz, DMSO-d₆): 8.20 (s, 1H), 7.58 (s, 1H), 7.41-7.30 (m, 1H), 7.25-7.17 (m, 1H), 7.15-7.00 (m, 2H), 5.76 (quint, J = 6.8 Hz, 1H), 3.49 (s, 3H), 3.47 (s, 3H), 3.43-3.38 (m, 2H), 3.23-3.15 (m, 1H), 3.02-2.87 (m, 1H), 2.71- 2.57 (m, 4H), 2.42 (s, 3H), 2.17-2.06 (m, 1H), 1.72-1.60 (m, 1H), 1.53 (d, J = 6.8 Hz, 3H) NH not observed. |

### Example 3. Biological Analysis of Disclosed Compounds

The capacity of compounds to inhibit SOS1 binding to KRAS-WT (wild-type) was quantified using a FRET-based protein-protein interaction assay. The assay is based on the transfer of energy between two fluorophores, a donor and an acceptor, when in close proximity. In this instance, the donor is a Europium-conjugated α-GST antibody that binds to GST-tagged KRAS-WT, and the acceptor is an XL665-conjugated α-His 6 antibody that binds to His 6-tagged SOS1. Binding of SOS1 to KRAS-WT results in an increased fluorescent signal at emission wavelength of 665 nm which can be detected on the EnVision plate reader. Compounds that inhibit binding will reduce the 665 nm signal emitted. Recombinant KRAS-WT protein (40 nM; Human KRAS, aa1-188 recombinant protein with N-terminal GST-tag) and SOS1 protein (40 nM; Human SOS1 exchange domain, aa564-1049 with N-terminal 6His-tag) were mixed together in assay buffer (5 mM HEPES pH7.3, 150 mM NaCl, 10 mM EDTA, 5 mM MgCl₂, 0.05% BSA, 0.0025% NP-40, 1 mM DTT and 100 mM KF) and incubated at room temperature with a dose response of compound in a 384-well low volume white plate and a final volume of 5 ul. After a 60 minute incubation, 5 ul of 4 nM anti-GST-Eu(K) (Cisbio, France) combined with 20 nM anti-6His-XL665 (Cisbio, France), diluted in assay buffer, was added to the plate. Following a further 4 hr incubation at room temperature, time-resolved fluorescence was measured on the EnVision plate reader. DMSO (0.05%) and 10 μM reference compound were used to generate the Max and Min assay signals, respectively. Data was analysed using a four-parameter logistic model to calculate IC₅₀ values, with at least two independent replicates performed for each compound.

Data was analyzed using a four-parameter logistic model to calculate IC₅₀ values, with at least two independent replicates performed for each compound.

TABLE 5
SOS1/KRAS WT HTRF binding assay data

| | | | SOS1/KRAS WT |
|---|---|---|---|
| | | | HTRF Binding |
| | Name | Structure | assay pIC50 |
| 1 | 3-cyclopentyl-1,8-dimethyl-5-[1- [3- (trifluoromethyl)phenyl]ethylamino] imidazo[4,5-g]phthalazin-2-one | 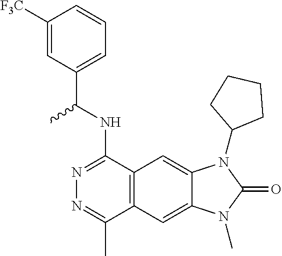 [SMILES: Cc1nnc(NC(C)c2cccc(C(F)(F)F)c2)c2cc3c(cc12)n(C)c(=O)n3C1CCCC1] | 7.43 |
| 2 | 3-cyclopentyl-1,8-dimethyl-5- [[(1R)-1-[3- (trifluoromethyl)phenyl]ethyl]amino] imidazo[4,5-g]phthalazin-2-one | 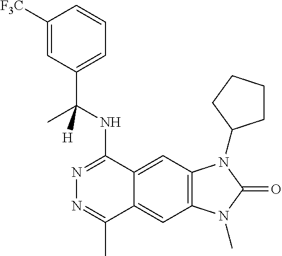 [SMILES: Cc1nnc(N[C@H](C)c2cccc(C(F)(F)F)c2)c2cc3c(cc12)n(C)c(=O)n3C1CCCC1] | 7.88 |
| 3 | 1,3,8-trimethyl-5-[[(1R)-1-[3- (trifluoromethyl)phenyl]ethyl]amino] imidazo[4,5-g]phthalazin-2-one | 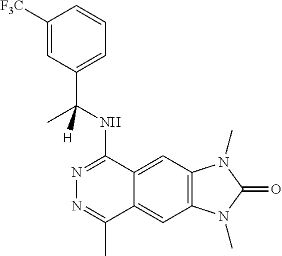 [SMILES: Cc1nnc(N[C@H](C)c2cccc(C(F)(F)F)c2)c2cc3c(cc12)n(C)c(=O)n3C] | 7.68 |
| 4 | 5-[[(1R)-1-[3-amino-5- (trifluoromethyl)phenyl]ethyl] amino]-1,3,8-trimethyl-imidazo[4,5- g]phthalazin-2-one | 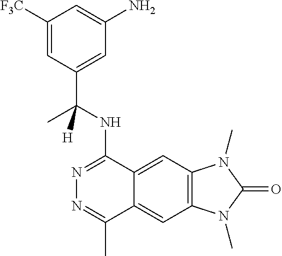 [SMILES: Cc1nnc(N[C@H](C)c2cc(N)cc(C(F)(F)F)c2)c2cc3c(cc12)n(C)c(=O)n3C] | 7.77 |
| 5 | 5-[[(1R)-1-[3- (difluoromethyl)phenyl]ethyl]amino]- 1,3,8-trimethyl-imidazo[4,5- g]phthalazin-2-one | 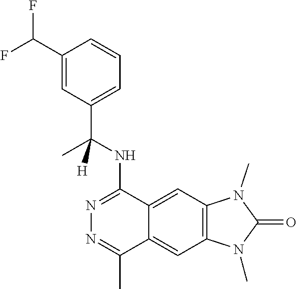 [SMILES: Cc1nnc(N[C@H](C)c2cccc(C(F)F)c2)c2cc3c(cc12)n(C)c(=O)n3C] | 7.40 |
| 6 | 5-[[(1R)-1-[3-(1,1-difluoro-2- hydroxy- ethyl)phenyl]ethyl]amino]-1,3,8- trimethyl-imidazo[4,5- g]phthalazin-2-one | 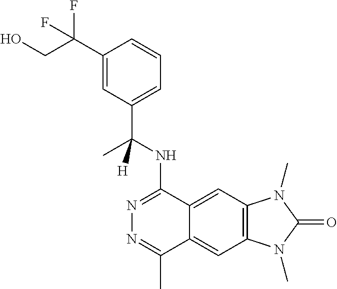 [SMILES: Cc1nnc(N[C@H](C)c2cccc(C(F)(F)CO)c2)c2cc3c(cc12)n(C)c(=O)n3C] | 7.73 |
| 7 | 5-[[(1R)-1-[3-(1,1-difluoro-2- hydroxy-ethyl)-2-fluoro- phenyl]ethyl]amino]-1,3,8- trimethyl-imidazo[4,5- g]phthalazin-2-one | 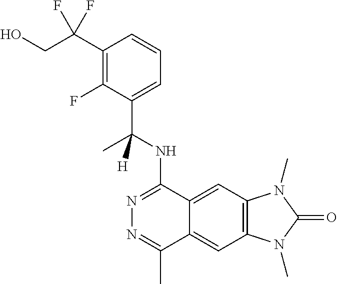 [SMILES: Cc1nnc(N[C@H](C)c2cccc(C(F)(F)CO)c2F)c2cc3c(cc12)n(C)c(=O)n3C] | 7.76 |
| 8 | 3-(2-methoxyethyl)-1,8-dimethyl- 5-[[(1R)-1-[3- (trifluoromethyl)phenyl]ethyl] amino]imidazo[4,5-g]phthalazin-2-one | 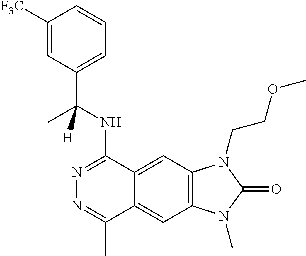 [SMILES: COCCn1c(=O)n(C)c2cc3c(C)nnc(N[C@H](C)c4cccc(C(F)(F)F)c4)c3cc21] | 7.90 |
| 9 | 3-ethyl-1,8-dimethyl-5-[[(1R)-1-[3- (trifluoromethyl)phenyl]ethyl]amino] imidazo[4,5-g]phthalazin-2-one | 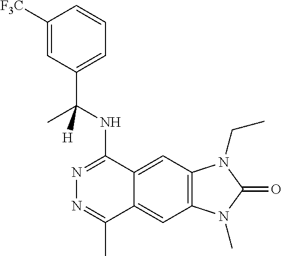 [SMILES: CCn1c(=O)n(C)c2cc3c(C)nnc(N[C@H](C)c4cccc(C(F)(F)F)c4)c3cc21] | 7.92 |
| 10 | 5-[[(1R)-1-[3-(1,1-difluoro-2- hydroxy-ethyl)-2-methyl- phenyl]ethyl]amino]-1,3,8- trimethyl-imidazo[4,5- g]phthalazin-2-one | 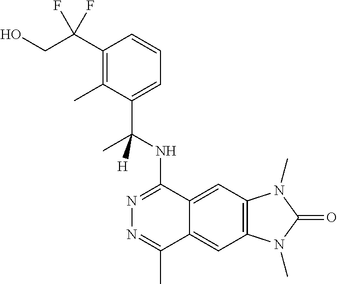 [SMILES: Cc1c([C@@H](C)Nc2nnc(C)c3cc4c(cc23)n(C)c(=O)n4C)cccc1C(F)(F)CO] | 8.04 |

| | | | |
|---|---|---|---|
| 11 | 1,8-dimethyl-5-[[(1R)-1-[3- (trifluoromethyl)phenyl]ethyl] amino]-3-(2,2,2- trifluoroethyl)imidazo[4,5- g]phthalazin-2-one | 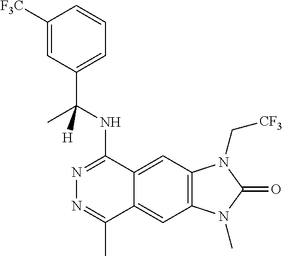 [SMILES: Cc1nnc(N[C@H](C)c2cccc(C(F)(F)F)c2)c2cc3c(cc12)n(C)c(=O)n3CC(F)(F)F] | 7.92 |

| | | | |
|---|---|---|---|
| 12 | 3-isopropyl-1,8-dimethyl-5-[[(1R)- 1-[3- (trifluoromethyl)phenyl]ethyl] amino]imidazo[4,5-g]phthalazin-2-one | 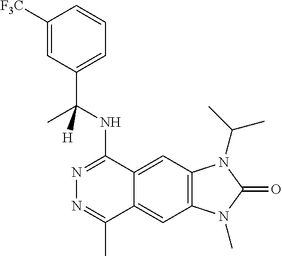 [SMILES: Cc1nnc(N[C@H](C)c2cccc(C(F)(F)F)c2)c2cc3c(cc12)n(C)c(=O)n3C(C)C] | 8.09 |

| | | | |
|---|---|---|---|
| 13 | 3-cyclopropyl-1,8-dimethyl-5- [[(1R)-1-[3- (trifluoromethyl)phenyl]ethyl] amino]imidazo[4,5-g]phthalazin-2-one | 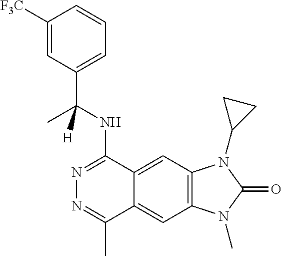 [SMILES: Cc1nnc(N[C@H](C)c2cccc(C(F)(F)F)c2)c2cc3c(cc12)n(C)c(=O)n3C1CC1] | 7.94 |

| | | | |
|---|---|---|---|
| 14 | 3-isopropyl-1,8-dimethyl-5-[[(1R)- 1-[3-(1,1-difluoro-2-hydroxy- ethyl)phenyl]ethyl]amino]imidazo [4,5-g]phthalazin-2-one | 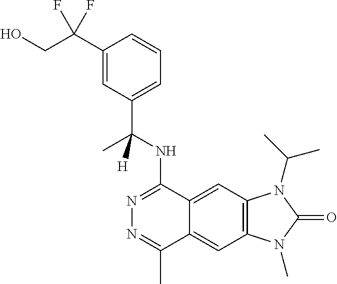 [SMILES: Cc1nnc(N[C@H](C)c2cccc(C(F)(F)CO)c2)c2cc3c(cc12)n(C)c(=O)n3C(C)C] | 8.12 |

| | | | |
|---|---|---|---|
| 15 | 3-isopropyl-1,8-dimethyl-5-[[(1R)- 1-[3-(1,1-difluoro-2-hydroxy- ethyl)-2-fluoro- phenyl]ethyl]amino]imidazo[4,5- g]phthalazin-2-one | 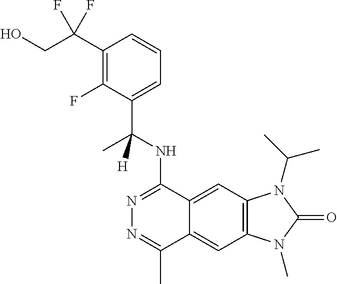 [SMILES: Cc1nnc(N[C@H](C)c2cccc(C(F)(F)CO)c2F)c2cc3c(cc12)n(C)c(=O)n3C(C)C] | 8.08 |

| | | | |
|---|---|---|---|
| 16 | 1,3,8-trimethyl-5-[[(1R)-1-[3-(1,1- difluoro-2-hydroxy-2-methyl- propyl)-2-fluoro- phenyl]ethyl]amino]imidazo[4,5- g]phthalazin-2-one | 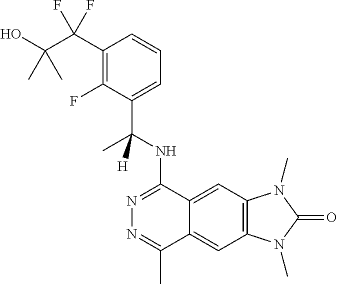 [SMILES: Cc1nnc(N[C@H](C)c2cccc(C(F)(F)C(C)(C)O)c2F)c2cc3c(cc12)n(C)c(=O)n3C] | 7.95 |

| | | | |
|---|---|---|---|
| 17 | 1-(2-methoxyethyl)-3,8-dimethyl- 5-[[(1R)-1-[3- (trifluoromethyl)phenyl]ethyl]amino] imidazo[4,5-g]phthalazin-2-one | 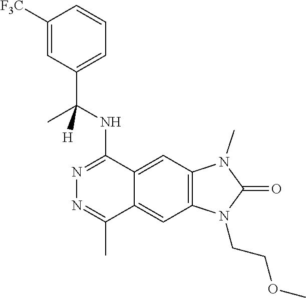 [SMILES: COCCn1c(=O)n(C)c2cc3c(N[C@H](C)c4cccc(C(F)(F)F)c4)nnc(C)c3cc21] | 7.64 |

| | | | |
|---|---|---|---|
| 18 | 3-(cyclopropylmethyl)-1,8- dimethyl-5-[[(1R)-1-[3- (trifluoromethyl)phenyl]ethyl]amino] imidazo[4,5-g]phthalazin-2-one | 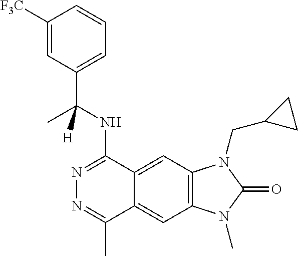 [SMILES: Cc1nnc(N[C@H](C)c2cccc(C(F)(F)F)c2)c2cc3c(cc12)n(C)c(=O)n3CC1CC1] | 7.63 |

| | | | |
|---|---|---|---|
| 19 | 3-ethyl-1,8-dimethyl-5-[[(1R)-1- [3-(1,1-difluoro-2-hydroxy-ethyl)- 2-fluoro- phenyl]ethyl]amino]imidazo[4,5- g]phthalazin-2-one | 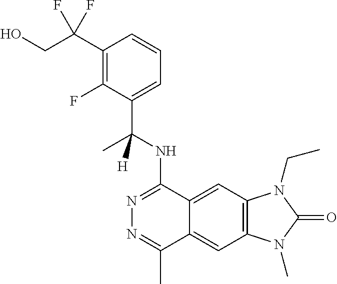 [SMILES: CCn1c(=O)n(C)c2cc3c(C)nnc(N[C@H](C)c4cccc(C(F)(F)CO)c4F)c3cc21] | 8.04 |

| | | | |
|---|---|---|---|
| 20 | 3-ethyl-1,8-dimethyl-5-[[(1R)-1- [3-(difluoromethyl)-2-fluoro- phenyl]ethyl]amino]imidazo[4,5- g]phthalazin-2-one | 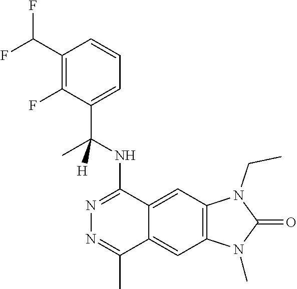 [SMILES: CCn1c(=O)n(C)c2cc3c(C)nnc(N[C@H](C)c4cccc(C(F)F)c4F)c3cc21] | 8.00 |

| | | | |
|---|---|---|---|
| 21 | 3-cyclobutyl-1,8-dimethyl-5- [[(1R)-1-[3-(1,1-difluoro-2- hydroxy-ethyl)-2-fluoro- phenyl]ethyl]amino]imidazo[4,5- g]phthalazin-2-one | 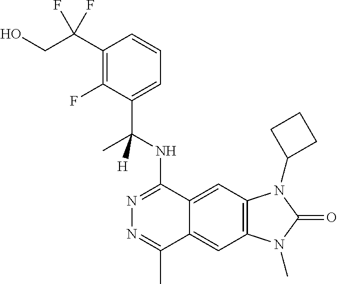 [SMILES: Cc1nnc(N[C@H](C)c2cccc(C(F)(F)CO)c2F)c2cc3c(cc12)n(C)c(=O)n3C1CCC1] | 8.01 |

| | | | |
|---|---|---|---|
| 22 | 3-cyclobutyl-1,8-dimethyl-5- [[(1R)-1-[3- (trifluoromethyl)phenyl]ethyl]amino] imidazo[4,5-g]phthalazin-2-one | 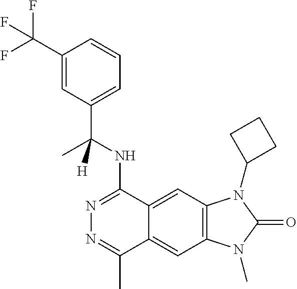 [SMILES: Cc1nnc(N[C@H](C)c2cccc(C(F)(F)F)c2)c2cc3c(cc12)n(C)c(=O)n3C1CCC1] | 7.94 |

| | | | |
|---|---|---|---|
| 23 | 4,9-dimethyl-6-[[(1R)-1-[3- (trifluoromethyl)phenyl]ethyl] amino]pyridazino[4,5- g][1,4]benzoxazin-3-one | 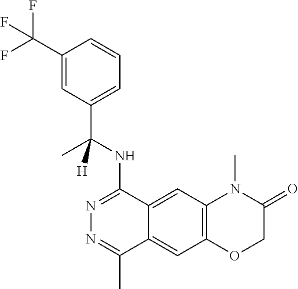 [SMILES: Cc1nnc(N[C@H](C)c2cccc(C(F)(F)F)c2)c2cc3c(cc12)OCC(=O)N3C] | 7.35 |

| | | |
|---|---|---|
| 24 | 4′,9′-dimethyl-6′-[[(1R)-1-[3- (trifluoromethyl)phenyl]ethyl] amino]spiro[cyclopropane-1,2′- pyridazino[4,5- g][1,4]benzoxazine]-3′-one | 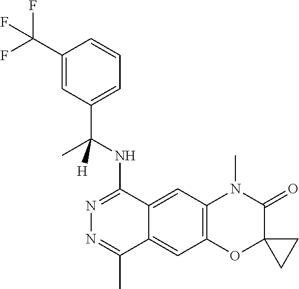 [SMILES: Cc1nnc(N[C@H](C)c2cccc(C(F)(F)F)c2)c2cc3c(cc12)OC1(CC1)C(=O)N3C] |

| | | | |
|---|---|---|---|
| 25 | 4′-isopropyl-9′-methyl-6′-[[(1R)-1-[3- (trifluoromethyl)phenyl]ethyl] amino]spiro[cyclopropane-1,2′- pyridazino[4,5- g][1,4]benzoxazine]-3′-one | 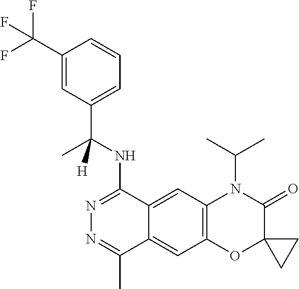 [SMILES: Cc1nnc(N[C@H](C)c2cccc(C(F)(F)F)c2)c2cc3c(cc12)OC1(CC1)C(=O)N3C(C)C] | 7.20 |

| | | | |
|---|---|---|---|
| 26 | 3-tert-butyl-1,8-dimethyl-5-[[(1R)- 1-[3-(1,1-difluoro-2-hydroxy- ethyl)-2-fluoro- phenyl]ethyl]amino]imidazo[4,5- g]phthalazin-2-one | 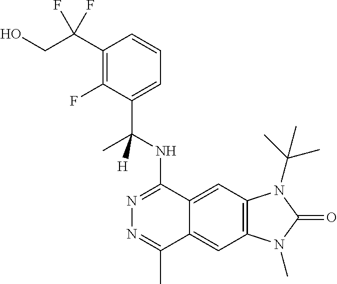 [SMILES: Cc1nnc(N[C@H](C)c2cccc(C(F)(F)CO)c2F)c2cc3c(cc12)n(C)c(=O)n3C(C)(C)C] | 7.84 |

| | | | |
|---|---|---|---|
| 27 | 3-tert-butyl-1,8-dimethyl-5-[[(1R)- 1-[3-(difluoromethyl)-2-fluoro- phenyl]ethyl]amino]imidazo[4,5- g]phthalazin-2-one | 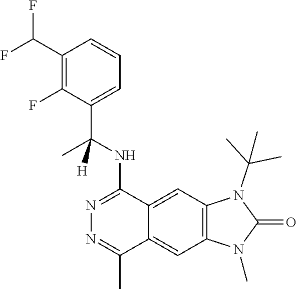 [SMILES: Cc1nnc(N[C@H](C)c2cccc(C(F)F)c2F)c2cc3c(cc12)n(C)c(=O)n3C(C)(C)C] | 8.03 |

| | | | |
|---|---|---|---|
| 28 | 3-(2-methoxyethyl)-1,8-dimethyl- 5-[[(1R)-1-[3-(difluoromethyl)-2- fluoro- phenyl]ethyl]amino]imidazo[4,5- g]phthalazin-2-one | 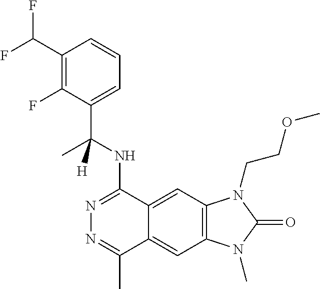 [SMILES: COCCn1c(=O)n(C)c2cc3c(C)nnc(N[C@H](C)c4cccc(C(F)F)c4F)c3cc21] | 7.91 |

| | | |
|---|---|---|
| 29 | 2,2,4,9-tetramethyl-6-[[(1R)-1-[3- (trifluoromethyl)phenyl]ethyl]amino] pyridazino[4,5- g][1,4]benzoxazin-3-one | 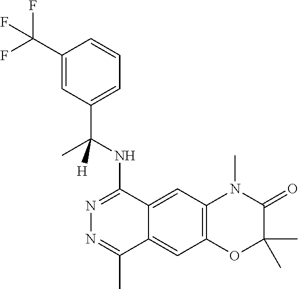 [SMILES: Cc1nnc(N[C@H](C)c2cccc(C(F)(F)F)c2)c2cc3c(cc12)OC(C)(C)C(=O)N3C] |

| | | | |
|---|---|---|---|
| 30 | 2,2,4,9-tetramethyl-6-[[(1R)-1-[3- (difluoromethyl)-2-fluoro- phenyl]ethyl]amino]pyridazino[4,5 -g][1,4]benzoxazin-3-one | 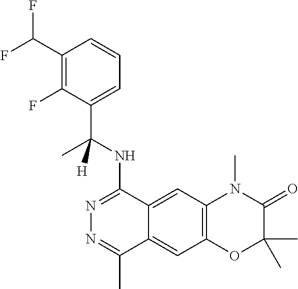 [SMILES: Cc1nnc(N[C@H](C)c2cccc(C(F)F)c2F)c2cc3c(cc12)OC(C)(C)C(=O)N3C] | 7.83 |

| | | | |
|---|---|---|---|
| 31 | 1,3,8-trimethyl-5-[[(1R)-1-[3- (difluoromethyl)-2-fluoro- phenyl]ethyl]amino]imidazo[4,5- g]phthalazin-2-one | 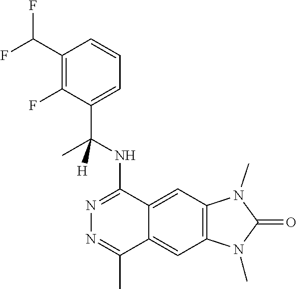 [SMILES: Cc1nnc(N[C@H](C)c2cccc(C(F)F)c2F)c2cc3c(cc12)n(C)c(=O)n3C] | 8.02 |

| | | | |
|---|---|---|---|
| 32 | 1,3,3,5-tetramethyl-8-[[(1R)-1-[3- (trifluoromethyl)phenyl]ethyl]amino] pyrrolo[2,3-g]phthalazin-2-one | 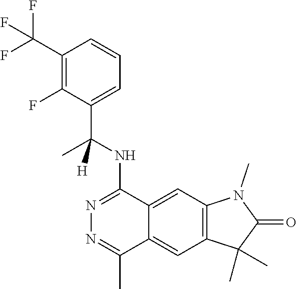 [SMILES: Cc1nnc(N[C@H](C)c2cccc(C(F)(F)F)c2F)c2cc3c(cc12)C(C)(C)C(=O)N3C] | 7.97 |

| | | | |
|---|---|---|---|
| 33 | 3-cyclopropyl-1,8-dimethyl-5- [[(1R)-1-[3-(1,1-difluoro-2- hydroxy-2-methyl-propyl)-2- fluoro- phenyl]ethyl]amino]imidazo[4,5- g]phthalazin-2-one | 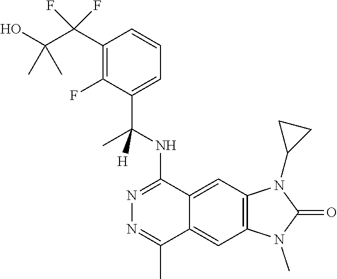 [SMILES: Cc1nnc(N[C@H](C)c2cccc(C(F)(F)C(C)(C)O)c2F)c2cc3c(cc12)n(C)c(=O)n3C1CC1] | 8.01 |

| | | | |
|---|---|---|---|
| 34 | 3-cyclopropyl-1,8-dimethyl-5- [[(1R)-1-[3-(difluoromethyl)-2- fluoro- phenyl]ethyl]amino]imidazo[4,5- g]phthalazin-2-one | 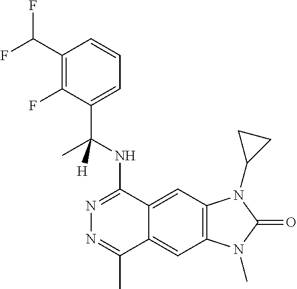 [SMILES: Cc1nnc(N[C@H](C)c2cccc(C(F)F)c2F)c2cc3c(cc12)n(C)c(=O)n3C1CC1] | 8.01 |

| | | | |
|---|---|---|---|
| 35 | 3-(2-methoxyethyl)-1,8-dimethyl- 5-[[(1R)-1-[2-fluoro-3- (trifluoromethyl)phenyl]ethyl]amino] imidazo[4,5-g]phthalazin-2-one | 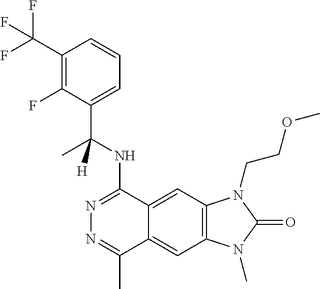 [SMILES: COCCn1c(=O)n(C)c2cc3c(C)nnc(N[C@H](C)c4cccc(C(F)(F)F)c4F)c3cc21] | 7.97 |

| | | | |
|---|---|---|---|
| 36 | 5-[[(1R)-1-[3-(1,1-difluoro-2- methoxy-ethyl)-2-fluoro- phenyl]ethyl]amino]-1,3,8- trimethyl-imidazo[4,5- g]phthalazin-2-one | 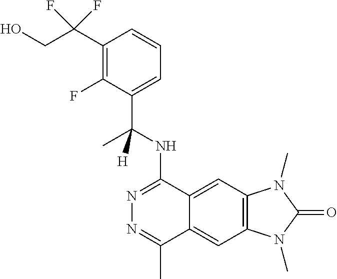 [SMILES: Cc1nnc(N[C@H](C)c2cccc(C(F)(F)CO)c2F)c2cc3c(cc12)n(C)c(=O)n3C] | 7.92 |

| | | | |
|---|---|---|---|
| 37 | 3-(2-methoxyethyl)-1,8-dimethyl- 5-[[(1R)-1-[3-(1,1-difluoro-2- hydroxy-ethyl)-2-fluoro- phenyl]ethyl]amino]imidazo[4,5- g]phthalazin-2-one | 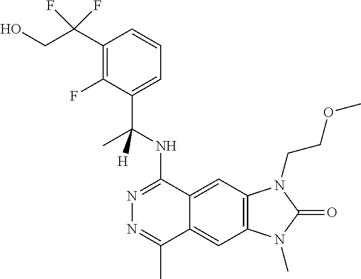 [SMILES: COCCn1c(=O)n(C)c2cc3c(C)nnc(N[C@H](C)c4cccc(C(F)(F)CO)c4F)c3cc21] | 7.98 |

| | | | |
|---|---|---|---|
| 38 | 3-isopropyl-1,8-dimethyl-5-[[(1R)- 1-[2-fluoro-3- (trifluoromethyl)phenyl]ethyl]amino] imidazo[4,5-g]phthalazin-2-one | 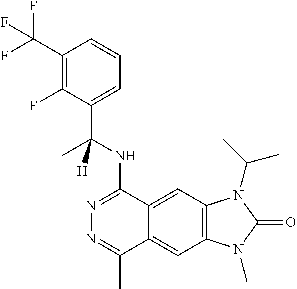 [SMILES: Cc1nnc(N[C@H](C)c2cccc(C(F)(F)F)c2F)c2cc3c(cc12)n(C)c(=O)n3C(C)C] | 8.12 |

| | | | |
|---|---|---|---|
| 39 | 2-methyl-3-[(1R)-1-[(1,3,8- trimethyl-2-oxo-imidazo[4,5- g]phthalazin-5- yl)amino]ethyl]benzonitrile | 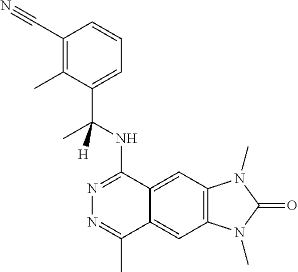 [SMILES: Cc1c(C#N)cccc1[C@@H](C)Nc1nnc(C)c2cc3c(cc12)n(C)c(=O)n3C] | 7.35 |

| | | | |
|---|---|---|---|
| 40 | 1,3,8-trimethyl-5-[[(1R)-1-[2- fluoro-3- (trifluoromethyl)phenyl]ethyl] amino]imidazo[4,5-g]phthalazin-2-one | 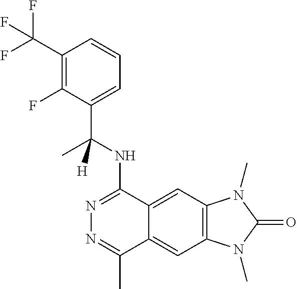 [SMILES: Cc1nnc(N[C@H](C)c2cccc(C(F)(F)F)c2F)c2cc3c(cc12)n(C)c(=O)n3C] | 8.05 |

| | | | |
|---|---|---|---|
| 41 | 1,3,8-trimethyl-5-[[(1R)-1-[3-(2- amino-1,1-difluoro-ethyl)-2- fluoro- phenyl]ethyl]amino]imidazo[4,5- g]phthalazin-2-one | 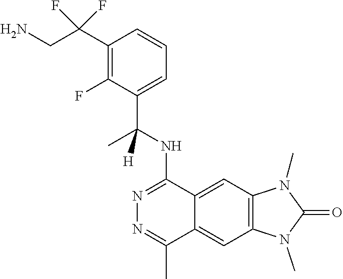 [SMILES: Cc1nnc(N[C@H](C)c2cccc(C(F)(F)CN)c2F)c2cc3c(cc12)n(C)c(=O)n3C] | 7.79 |

| | | | |
|---|---|---|---|
| 42 | 5,10-dimethyl-7-[[(1R)-1-[3- (trifluoromethyl)phenyl]ethyl]amino]- 2,3-dihydropyridazino[4,5- h][1,5]benzoxazepin-4-one | 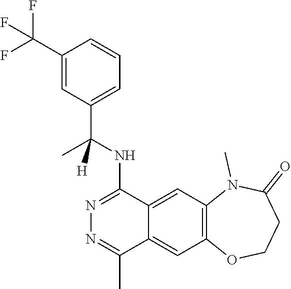 [SMILES: Cc1nnc(N[C@H](C)c2cccc(C(F)(F)F)c2)c2cc3c(cc12)OCCC(=O)N3C] | 7.46 |

| | | | |
|---|---|---|---|
| 43 | 3-isopropyl-1,8-dimethyl-5-[[(1R)- 1-[3-(difluoromethyl)-2-fluoro- phenyl]ethyl]amino]imidazo[4,5- g]phthalazin-2-one | 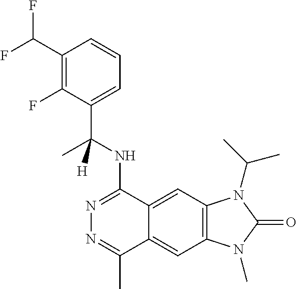 [SMILES: Cc1nnc(N[C@H](C)c2cccc(C(F)F)c2F)c2cc3c(cc12)n(C)c(=O)n3C(C)C] | 8.55 |

| | | | |
|---|---|---|---|
| 44 | 1,3,3,5-tetramethyl-8-[[(1R)-1-[3- (difluoromethyl)-2-fluoro- phenyl]ethyl]amino]pyrrolo[2,3- g]phthalazin-2-one | 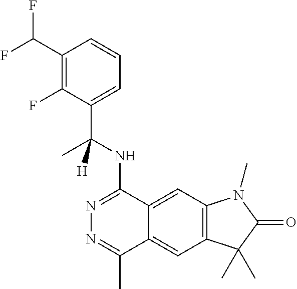 [SMILES: Cc1nnc(N[C@H](C)c2cccc(C(F)F)c2F)c2cc3c(cc12)C(C)(C)C(=O)N3C] | 8.06 |

| | | | |
|---|---|---|---|
| 45 | 2-methyl-3-[(1R)-1-[(3-isopropyl- 1,8-dimethyl-2-oxo-imidazo[4,5- g]phthalazin-5- yl)amino]ethyl]benzonitrile | 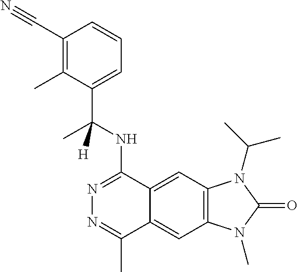 [SMILES: Cc1c(C#N)cccc1[C@@H](C)Nc1nnc(C)c2cc3c(cc12)n(C(C)C)c(=O)n3C] | 8.01 |

| | | | |
|---|---|---|---|
| 46 | 1,3,3,5-tetramethyl-8-[[(1R)-1-[3- (1,1-difluoro-2-hydroxy-2-methyl- propyl)-2-fluoro- phenyl]ethyl]amino]pyrrolo[2,3- g]phthalazin-2-one | 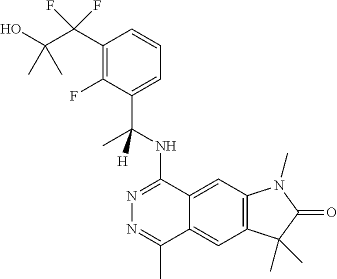 [SMILES: Cc1nnc(N[C@H](C)c2cccc(C(F)(F)C(C)(C)O)c2F)c2cc3c(cc12)C(C)(C)C(=O)N3C] | 7.96 |

| | | | |
|---|---|---|---|
| 47 | 2-methyl-3-[(1R)-1-[(3- cyclopropyl-1,8-dimethyl-2-oxo- imidazo[4,5-g]phthalazin-5- yl)amino]ethyl]benzonitrile | 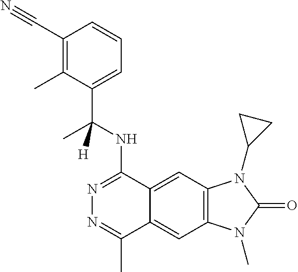 [SMILES: Cc1c(C#N)cccc1[C@@H](C)Nc1nnc(C)c2cc3c(cc12)n(C1CC1)c(=O)n3C] | 7.86 |

| | | | |
|---|---|---|---|
| 48 | 2-methyl-3-[(1R)-1-[(1,3,3,5- tetramethyl-2-oxo-pyrrolo[3,2- g]phthalazin-8- yl)amino]ethyl]benzonitrile | 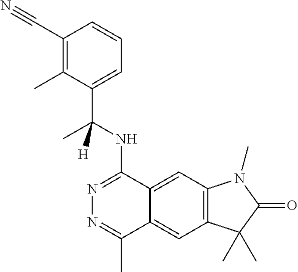 [SMILES: Cc1c(C#N)cccc1[C@@H](C)Nc1nnc(C)c2cc3c(cc12)N(C)C(=O)C3(C)C] | 7.87 |

| | | | |
|---|---|---|---|
| 49 | 2-methyl-3-[(1R)-1-[(3-tert-butyl- 1,8-dimethyl-2-oxo-imidazo[4,5- g]phthalazin-5- yl)amino]ethyl]benzonitrile | 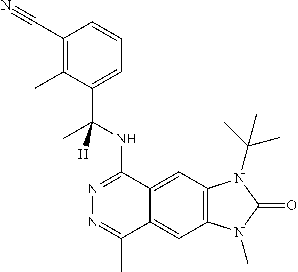 [SMILES: Cc1c(C#N)cccc1[C@@H](C)Nc1nnc(C)c2cc3c(cc12)n(C(C)(C)C)c(=O)n3C] | 7.95 |
| 50 | 3-(2,2-difluoroethyl)-1,8- dimethyl-5-[[(1R)-1-[3- (trifluoromethyl)phenyl]ethyl]amino] imidazo[4,5-g]phthalazin-2-one | 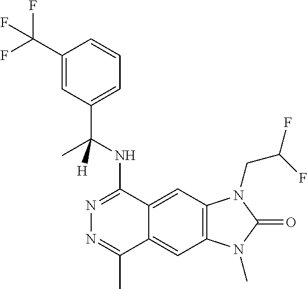 [SMILES: Cc1nnc(N[C@H](C)c2cccc(C(F)(F)F)c2)c2cc3c(cc12)n(C)c(=O)n3CC(F)F] | 7.88 |
| 51 | 5-[[(1R)-1-[3-(1,1-difluoro-2- hydroxy-ethyl)-5-fluoro- phenyl]ethyl]amino]-1,3,8- trimethyl-imidazo[4,5- g]phthalazin-2-one | 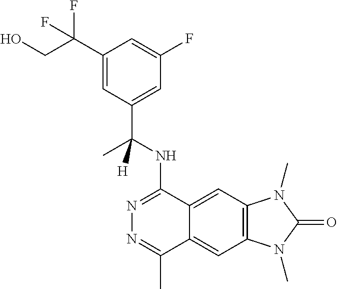 [SMILES: Cc1nnc(N[C@H](C)c2cc(F)cc(C(F)(F)CO)c2)c2cc3c(cc12)n(C)c(=O)n3C] | 7.63 |
| 52 | 3-cyclopropyl-1,8-dimethyl-5- [[(1R)-1-[2-fluoro-3- (trifluoromethyl)phenyl]ethyl]amino] imidazo[4,5-g]phthalazin-2-one | 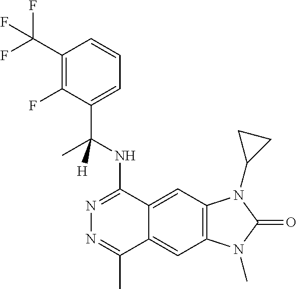 [SMILES: Cc1nnc(N[C@H](C)c2cccc(C(F)(F)F)c2F)c2cc3c(cc12)n(C)c(=O)n3C1CC1] | 8.09 |
| 53 | 3-tert-butyl-1,8-dimethyl-5-[[(1R)- 1-[3-(2-amino-1,1-difluoro-ethyl)- 2-fluoro- phenyl]ethyl]amino]imidazo[4,5- g]phthalazin-2-one | 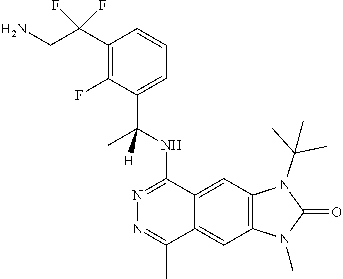 [SMILES: Cc1nnc(N[C@H](C)c2cccc(C(F)(F)CN)c2F)c2cc3c(cc12)n(C)c(=O)n3C(C)(C)C] | 8.30 |
| 54 | 1,3,8-trimethyl-5-[[(1R)-1-[2- methyl-3- (trifluoromethyl)phenyl]ethyl]amino] imidazo[4,5-g]phthalazin-2-one | 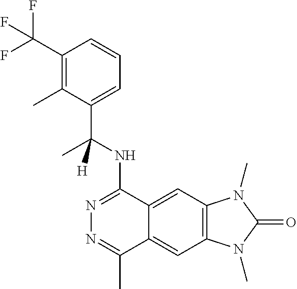 [SMILES: Cc1c([C@@H](C)Nc2nnc(C)c3cc4c(cc23)n(C)c(=O)n4C)cccc1C(F)(F)F] | 8.21 |
| 55 | 5-[[(1R)-1-[2-methoxy-3- (trifluoromethyl)phenyl]ethyl]amino]- 1,3,8-trimethyl-imidazo[4,5- g]phthalazin-2-one | 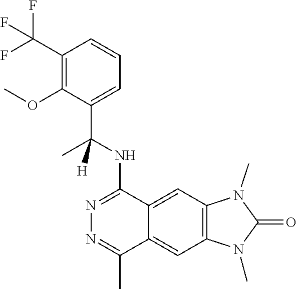 [SMILES: COc1c([C@@H](C)Nc2nnc(C)c3cc4c(cc23)n(C)c(=O)n4C)cccc1C(F)(F)F] | 7.24 |
| 56 | 1-cyclopropyl-3,8-dimethyl-5- [[(1R)-1-[3- (trifluoromethyl)phenyl]ethyl]amino] imidazo[4,5-g]phthalazin-2-one | 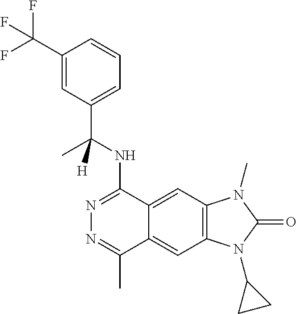 [SMILES: Cc1nnc(N[C@H](C)c2cccc(C(F)(F)F)c2)c2cc3c(cc12)n(C1CC1)c(=O)n3C] | 7.94 |
| 57 | 5-[[(1R)-1-(1H-indazol-6- yl)ethyl]amino]-1,3,8-trimethyl- imidazo[4,5-g]phthalazin-2-one | 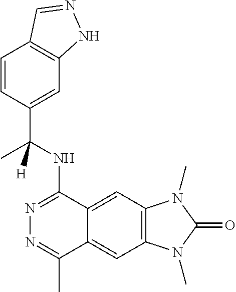 [SMILES: Cc1nnc(N[C@H](C)c2ccc3cn[nH]c3c2)c2cc3c(cc12)n(C)c(=O)n3C] | 7.03 |
| 58 | 3-cyclopropyl-1,8-dimethyl-5- [[1R)-1-[2-methyl-3- (trifluoromethyl)phenyl]ethyl]amino] imidazo[4,5-g]phthalazin-2-one | 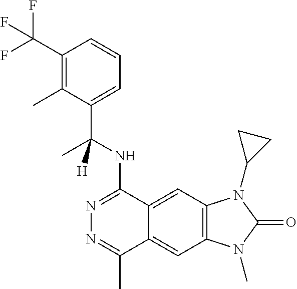 [SMILES: Cc1c([C@@H](C)Nc2nnc(C)c3cc4c(cc23)n(C2CC2)c(=O)n4C)cccc1C(F)(F)F] | 8.18 |
| 59 | 1-cyclopropyl-3,3,5-trimethyl-8- [[(1R)-1-[3-(1,1-difluoro-2- hydroxy-2-methyl-propyl)-2-fluoro- phenyl]ethyl]amino]pyrrolo[2,3- g]phthalazin-2-one | 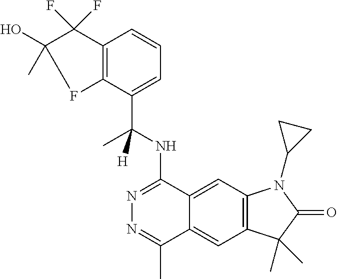 | 8.10 |
| 60 | 5-[[(1R)-1-(1H-indazol-4- yl)ethyl]amino]-1,3,8-trimethyl- imidazo[4,5-g]phthalazin-2-one | 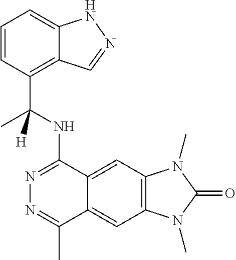 [SMILES: Cc1nnc(N[C@H](C)c2cccc3[nH]ncc23)c2cc3c(cc12)n(C)c(=O)n3C] | 6.68 |
| 61 | 1,3,8-trimethyl-5-[[(1R)-1-(3- fluoro-1-methyl-indol-7- yl)ethyl]amino]imidazo[4,5- g]phthalazin-2-one | 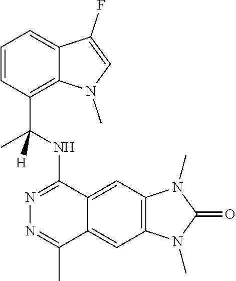 [SMILES: Cc1nnc(N[C@H](C)c2cccc3c(F)cn(C)c23)c2cc3c(cc12)n(C)c(=O)n3C] | 7.00 |
| 62 | 1-cyclopropyl-3,3,5-trimethyl-8- [[(1R)-1-[3-(difluoromethyl)-2- fluoro- phenyl]ethyl]amino]pyrrolo[2,3- g]phthalazin-2-one | 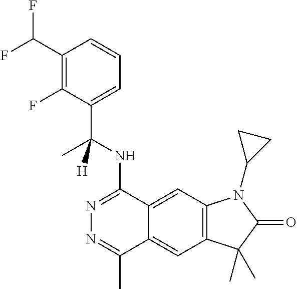 [SMILES: Cc1nnc(N[C@H](C)c2cccc(C(F)F)c2F)c2cc3c(cc12)C(C)(C)C(=O)N3C1CC1] | 7.97 |
| 63 | 1,3,8-trimethyl-5-[[(1R)-1-[3-[1,1- difluoro-2-(methylamino)ethyl]-2- fluoro- phenyl]ethyl]amino]imidazo[4,5- g]phthalazin-2-one | 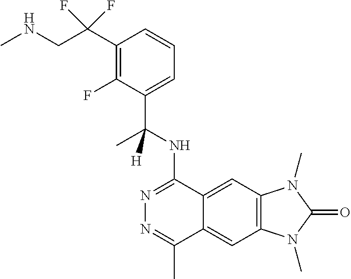 [SMILES: CNCC(F)(F)c1cccc([C@@H](C)Nc2nnc(C)c3cc4c(cc23)n(C)c(=O)n4C)c1F] | 7.80 |
| 64 | 1,3,8-trimethyl-5-[[(1R)-1-[3-(1,1- difluoro-2-hydroxy-2-methyl- propyl)phenyl]ethyl]amino]imidazo [4,5-g]phthalazin-2-one | 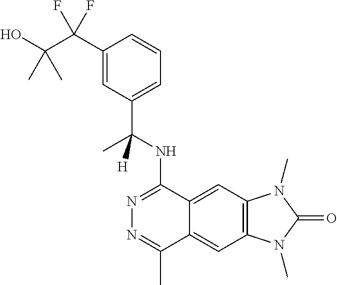 [SMILES: Cc1nnc(N[C@H](C)c2cccc(C(F)(F)C(C)(C)O)c2)c2cc3c(cc12)n(C)c(=O)n3C] | 8.09 |
| 65 | 1-cyclopropyl-3,3,5-trimethyl-8- [[(1R)-1-[3-(1,1-difluoro-2- hydroxy-2-methyl- propyl)phenyl]ethyl]amino]pyrrolo [2,3-g]phthalazin-2-one | 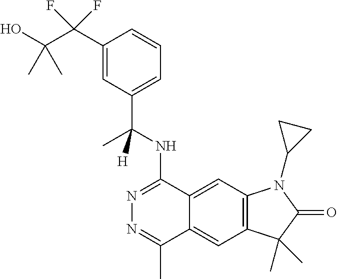 [SMILES: Cc1nnc(N[C@H](C)c2cccc(C(F)(F)C(C)(C)O)c2)c2cc3c(cc12)C(C)(C)C(=O)N3C1CC1] | 8.01 |
| 66 | 1-cyclopropyl-3,3,5-trimethyl-8- [[(1R)-1-[3-(1,1-difluoro-2- hydroxy-ethyl)-2-methyl- phenyl]ethyl]amino]pyrrolo[2,3- g]phthalazin-2-one | 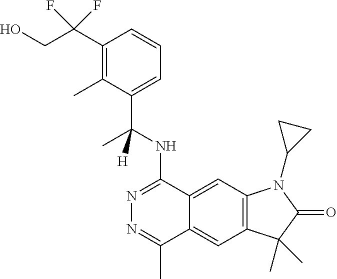 [SMILES: Cc1c([C@@H](C)Nc2nnc(C)c3cc4c(cc23)N(C2CC2)C(=O)C4(C)C)cccc1C(F)(F)CO] | 8.07 |
| 67 | 3-cyclopropyl-1,8-dimethyl-5- [[(1R)-1-[3-(1,1-difluoro-2- hydroxy-2-methyl- propyl)phenyl]ethyl]amino]imidazo [4,5-g]phthalazin-2-one | 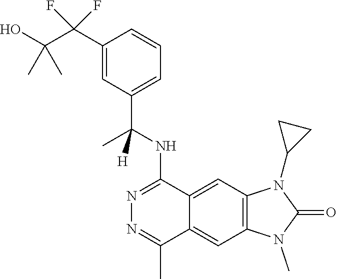 [SMILES: Cc1nnc(N[C@H](C)c2cccc(C(F)(F)C(C)(C)O)c2)c2cc3c(cc12)n(C)c(=O)n3C1CC1] | 8.13 |
| 68 | 3-cyclopropyl-1,8-dimethyl-5- [[(1R)-1-[3-(1,1-difluoro-2- hydroxy-ethyl)-2-methyl- phenyl]ethyl]amino]imidazo[4,5- g]phthalazin-2-one | 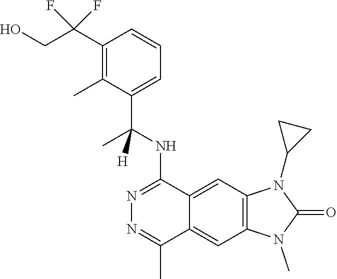 [SMILES: Cc1c([C@@H](C)Nc2nnc(C)c3cc4c(cc23)n(C2CC2)c(=O)n4C)cccc1C(F)(F)CO] | 8.02 |
| 69 | 1,3,3,5-tetramethyl-8-[[(1R)-1-[3- (1,1-difluoro-2-hydroxy-ethyl)-2- methyl- phenyl]ethyl]amino]pyrrolo[2,3- g]phthalazin-2-one | 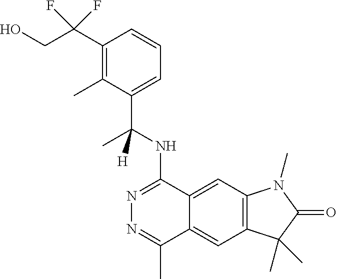 [SMILES: Cc1c([C@@H](C)Nc2nnc(C)c3cc4c(cc23)N(C)C(=O)C4(C)C)cccc1C(F)(F)CO] | 8.04 |
| 70 | 3-cyclopropyl-1,8-dimethyl-5- [[(1R)-1-[3-(1,1-difluoro-2- hydroxy- ethyl)phenyl]ethyl]amino]imidazo [4,5-g]phthalazin-2-one | 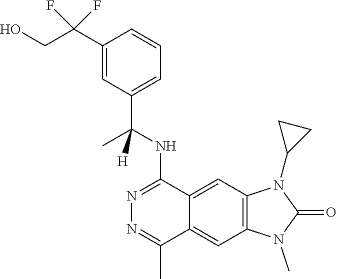 [SMILES: Cc1nnc(N[C@H](C)c2cccc(C(F)(F)CO)c2)c2cc3c(cc12)n(C)c(=O)n3C1CC1] | 7.94 |
| 72 | 1,5-dimethyl-8-[[(1R)-1-[3-(1,1- difluoro-2-hydroxy-ethyl)-2- methyl- phenyl]ethyl]amino]spiro[pyrrolo [2,3-g]phthalazine-3,4′- tetrahydropyran]-2-one | 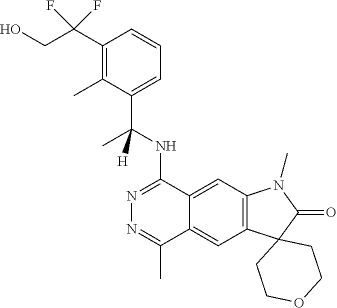 [SMILES: Cc1c([C@@H](C)Nc2nnc(C)c3cc4c(cc23)N(C)C(=O)C42CCOCC2)cccc1C(F)(F)CO] | 8.01 |
| 73 | 1,5-dimethyl-8-[[(1R)-1-[3-(1,1- difluoro-2-hydroxy-2-methyl- propyl)-2-fluoro- phenyl]ethyl]amino]spiro[pyrrolo [2,3-g]phthalazine-3,4′- tetrahydropyran]-2-one | 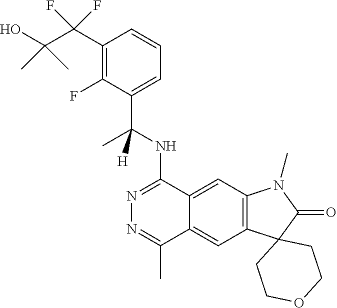 [SMILES: Cc1nnc(N[C@H](C)c2cccc(C(F)(F)C(C)(C)O)c2F)c2cc3c(cc12)C1(CCOCC1)C(=O)N3C] | 8.16 |
| 74 | 1,3,8-trimethyl-5-[[(1R)-1-[6- (trifluoromethyl)-2- pyridyl]ethyl]amino]imidazo[4,5- g]phthalazin-2-one | 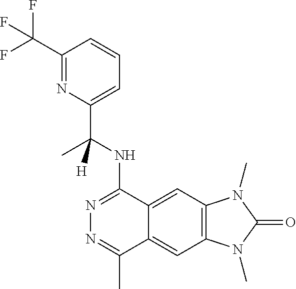 [SMILES: Cc1nnc(N[C@H](C)c2cccc(C(F)(F)F)n2)c2cc3c(cc12)n(C)c(=O)n3C] | 6.04 |
| 75 | 3-methoxy-1,3,5-trimethyl-8- [[(1R)-1-[3-(1,1-difluoro-2- hydroxy-2-methyl-propyl)-2- fluoro- phenyl]ethyl]amino]pyrrolo[2,3- g]phthalazin-2-one | 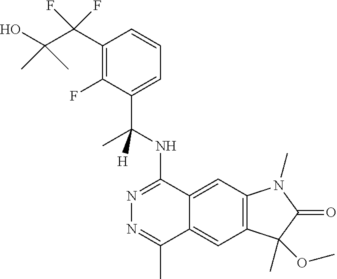 [SMILES: COC1(C)C(=O)N(C)c2cc3c(N[C@H](C)c4cccc(C(F)(F)C(C)(C)O)c4F)nnc(C)c3cc21] | 8.06 |
| 76 | 1,8-dimethyl-3-(1- methylcyclopropyl)-5-[[(1R)-1-[3- (1,1-difluoro-2-hydroxy-ethyl)-2- methyl- phenyl]ethyl]amino]imidazo[4,5- g]phthalazin-2-one | 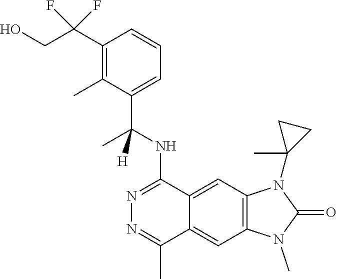 [SMILES: Cc1c([C@@H](C)Nc2nnc(C)c3cc4c(cc23)n(C2(C)CC2)c(=O)n4C)cccc1C(F)(F)CO] | 8.22 |
| 77 | 1,3,3,5-tetramethyl-8-[[(1R)-1-[3- (1,1-difluoro-2-hydroxy-2-methyl- propyl)phenyl]ethyl]amino]pyrrolo [2,3-g]phthalazin-2-one | 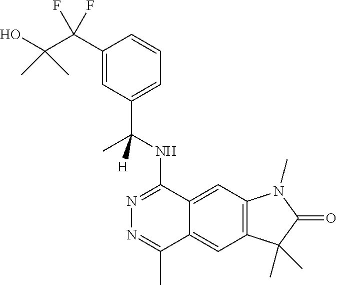 [SMILES: Cc1nnc(N[C@H](C)c2cccc(C(F)(F)C(C)(C)O)c2)c2cc3c(cc12)C(C)(C)C(=O)N3C] | 7.95 |
| 78 | 1,3,3,5-tetramethyl-8-[[(1R)-1-[3- (1,1-difluoro-2-hydroxy- ethyl)phenyl]ethyl]amino]pyrrolo [2,3-g]phthalazin-2-one | 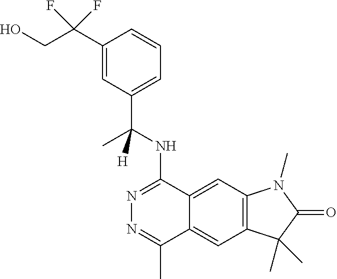 [SMILES: Cc1nnc(N[C@H](C)c2cccc(C(F)(F)CO)c2)c2cc3c(cc12)C(C)(C)C(=O)N3C] | 8.02 |
| 79 | 1-isopropyl-3,3,5-trimethyl-8- [[(1R)-1-[3-(1,1-difluoro-2- hydroxy-ethyl)-2-methyl- phenyl]ethyl]amino]pyrrolo[2,3- g]phthalazin-2-one | 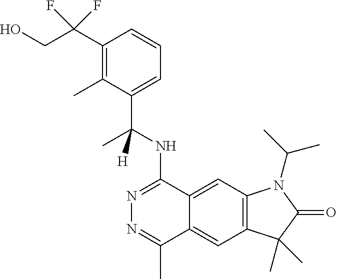 [SMILES: Cc1c([C@@H](C)Nc2nnc(C)c3cc4c(cc23)N(C(C)C)C(=O)C4(C)C)cccc1C(F)(F)CO] | 8.17 |
| 80 | 3-tert-butyl-1,8-dimethyl-5-[[(1R)- 1-[3-(1,1-difluoro-2-hydroxy- ethyl)phenyl]ethyl]amino]imidazo [4,5-g]phthalazin-2-one | 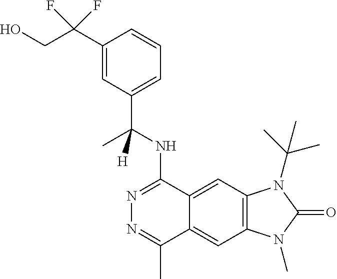 [SMILES: Cc1nnc(N[C@H](C)c2cccc(C(F)(F)CO)c2)c2cc3c(cc12)n(C)c(=O)n3C(C)(C)C] | 8.21 |
| 81 | 1-isopropyl-3,3,5-trimethyl-8- [[(1R)-1-[3-(1,1-difluoro-2- hydroxy-2-methyl- propyl)phenyl]ethyl]amino]pyrrol o[2,3-g]phthalazin-2-one | 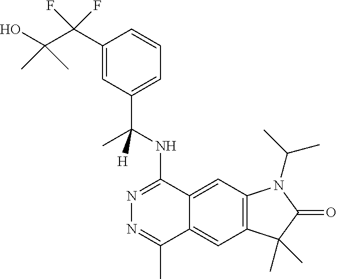 [SMILES: Cc1nnc(N[C@H](C)c2cccc(C(F)(F)C(C)(C)O)c2)c2cc3c(cc12)C(C)(C)C(=O)N3C(C)C] | 8.07 |
| 82 | 1-isopropyl-3,3,5-trimethyl-8- [[(1R)-1-[3-(1,1-difluoro-2- hydroxy- ethyl)phenyl]ethyl]amino]pyrrolo [2,3-g]phthalazin-2-one | 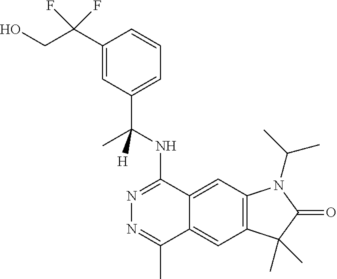 [SMILES: Cc1nnc(N[C@H](C)c2cccc(C(F)(F)CO)c2)c2cc3c(cc12)C(C)(C)C(=O)N3C(C)C] | 8.03 |
| 83 | 1,8-dimethyl-3-(1- methylcyclopropyl)-5-[[(1R)-1-[3- (1,1-difluoro-2-hydroxy-2-methyl- propyl)phenyl]ethyl]amino]imidazo [4,5-g]phthalazin-2-one | 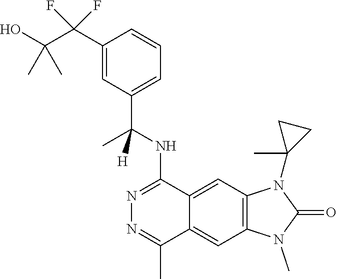 [SMILES: Cc1nnc(N[C@H](C)c2cccc(C(F)(F)C(C)(C)O)c2)c2cc3c(cc12)n(C)c(=O)n3C1(C)CC1] | 8.10 |
| 84 | 1,5-dimethyl-8-[[(1R)-1-[3-(1,1- difluoro-2-hydroxy-2-methyl- propyl)phenyl]ethyl]amino]spiro [pyrrolo[2,3-g]phthalazine-3,4′- tetrahydropyran]-2-one | 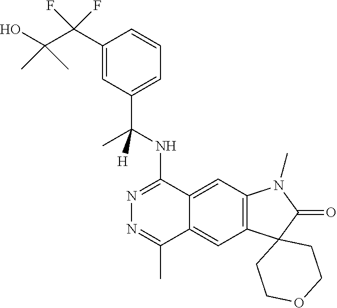 [SMILES: Cc1nnc(N[C@H](C)c2cccc(C(F)(F)C(C)(C)O)c2)c2cc3c(cc12)C1(CCOCC1)C(=O)N3C] | 8.05 |
| 85 | 3-isopropyl-1,8-dimethyl-5-[[(1R)- 1-[3-(1,1-difluoro-2-hydroxy- ethyl)-2-methyl- phenyl]ethyl]amino]imidazo[4,5- g]phthalazin-2-one | 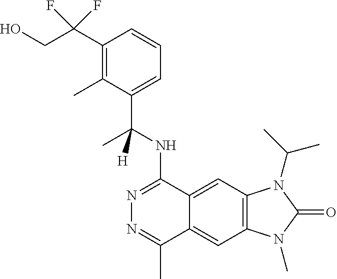 [SMILES: Cc1c([C@@H](C)Nc2nnc(C)c3cc4c(cc23)n(C(C)C)c(=O)n4C)cccc1C(F)(F)CO] | 8.08 |
| 86 | D8-[[(1R)-1-[3-(1,1-difluoro-2- hydroxy-2-methyl-propyl)-2- fluoro-phenyl]ethyl]amino]-3- (methoxymethyl)-1,3,5-trimethyl- pyrrolo[3,2-g]phthalazin-2-one | 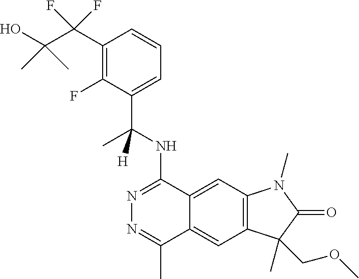 [SMILES: COCC1(C)C(=O)N(C)c2cc3c(N[C@H](C)c4cccc(C(F)(F)C(C)(C)O)c4F)nnc(C)c3cc21] | 7.99 |
| 87 | 3-isopropyl-1,8-dimethyl-5-[[(1R)- 1-[3-(1,1-difluoro-2-hydroxy-2- methyl- propyl)phenyl]ethyl]amino]imidazo [4,5-g]phthalazin-2-one | 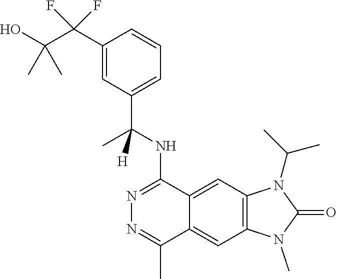 [SMILES: Cc1nnc(N[C@H](C)c2cccc(C(F)(F)C(C)(C)O)c2)c2cc3c(cc12)n(C)c(=O)n3C(C)C] | 8.07 |
| 88 | 1,5-dimethyl-8-[[(1R)-1-[3-(1,1- difluoro-2-hydroxy- ethyl)phenyl]ethyl]amino]spiro [pyrrolo[2,3-g]phthalazine-3,4′- tetrahydropyran]-2-one | 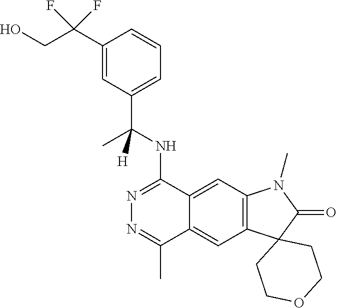 [SMILES: Cc1nnc(N[C@H](C)c2cccc(C(F)(F)CO)c2)c2cc3c(cc12)C1(CCOCC1)C(=O)N3C] | 8.11 |
| 89 | 1,5-dimethyl-8-[[(1R)-1-[3-(1,1- difluoro-2-hydroxy-ethyl)-2- fluoro- phenyl]ethyl]amino]spiro[pyrrolo [2,3-g]phthalazine-3,4′- tetrahydropyran]-2-one | 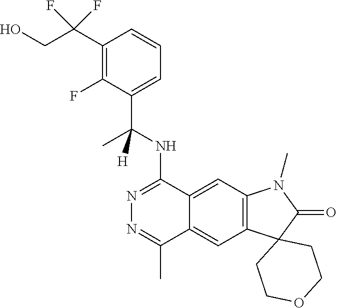 [SMILES: Cc1nnc(N[C@H](C)c2cccc(C(F)(F)CO)c2F)c2cc3c(cc12)C1(CCOCC1)C(=O)N3C] | 8.09 |
| 90 | 3-methoxy-1,3,5-trimethyl-8- [[(1R)-1-[3-(1,1-difluoro-2- hydroxy-2-methyl- propyl)phenyl]ethyl]amino]pyrrolo [2,3-g]phthalazin-2-one | 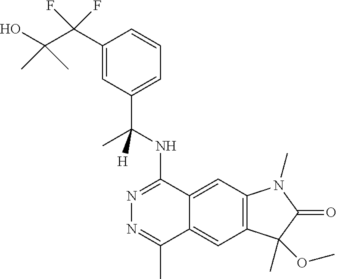 [SMILES: COC1(C)C(=O)N(C)c2cc3c(N[C@H](C)c4cccc(C(F)(F)C(C)(C)O)c4)nnc(C)c3cc21] | 7.97 |
| 91 | D8-[[(1R)-1-[3-(1,1-difluoro-2- hydroxy-2-methyl- propyl)phenyl]ethyl]amino]-3- (methoxymethyl)-1,3,5-trimethyl- pyrrolo[3,2-g]phthalazin-2-one | 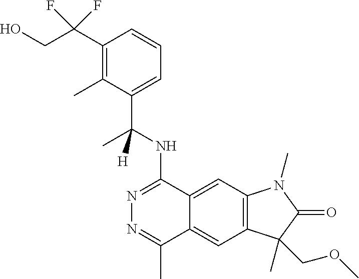 [SMILES: COCC1(C)C(=O)N(C)c2cc3c(N[C@H](C)c4cccc(C(F)(F)CO)c4C)nnc(C)c3cc21] | 7.75 |
| 92 | 3-methoxy-1,3,5-trimethyl-8- [[(1R)-1-[3-(1,1-difluoro-2- hydroxy-ethyl)-2-fluoro- phenyl]ethyl]amino]pyrrolo[2,3- g]phthalazin-2-one | 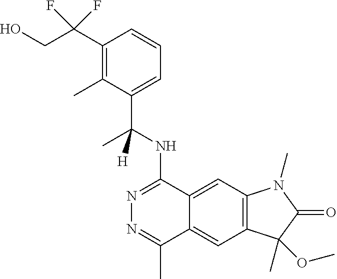 [SMILES: COC1(C)C(=O)N(C)c2cc3c(N[C@H](C)c4cccc(C(F)(F)CO)c4C)nnc(C)c3cc21] | 8.01 |
| 93 | 3-methoxy-1,3,5-trimethyl-8- [[(1R)-1-[3-(1,1-difluoro-2- hydroxy-ethyl)-2-fluoro- phenyl]ethyl]amino]pyrrolo[2,3- g]phthalazin-2-one | 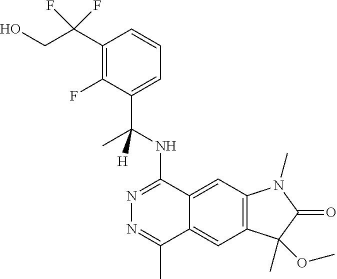 [SMILES: COC1(C)C(=O)N(C)c2cc3c(N[C@H](C)c4cccc(C(F)(F)CO)c4F)nnc(C)c3cc21] | 8.13 |
| 94 | 8-[[(1R)-1-[3-(1,1-difluoro-2- hydroxy-ethyl)-2-fluoro- phenyl]ethyl]amino]-3- (methoxymethyl)-1,3,5-trimethyl- pyrrolo[3,2-g]phthalazin-2-one | 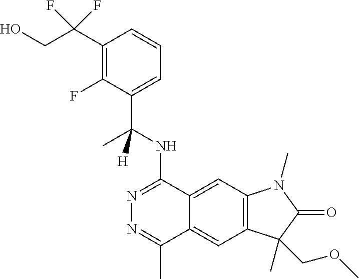 [SMILES: COCC1(C)C(=O)N(C)c2cc3c(N[C@H](C)c4cccc(C(F)(F)CO)c4F)nnc(C)c3cc21] | 8.05 |
| 95 | 1,3,3,8-tetramethyl-5-[[(1R)-1-[3- (1,1-difluoro-2-hydroxy- ethyl)phenyl]ethyl]amino]pyrrolo [3,2-g]phthalazin-2-one | 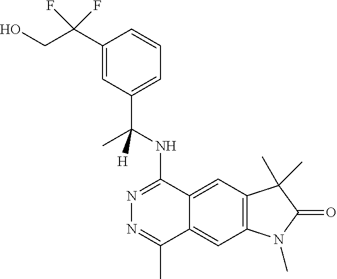 [SMILES: Cc1nnc(N[C@H](C)c2cccc(C(F)(F)CO)c2)c2cc3c(cc12)N(C)C(=O)C3(C)C] | 8.13 |
| 96 | 1,3,3,8-tetramethyl-5-[[(1R)-1-[3- (1,1-difluoro-2-hydroxy-2-methyl- propyl)phenyl]ethyl]amino]pyrrolo [3,2-g]phthalazin-2-one | 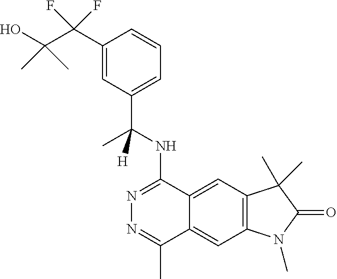 [SMILES: Cc1nnc(N[C@H](C)c2cccc(C(F)(F)C(C)(C)O)c2)c2cc3c(cc12)N(C)C(=O)C3(C)C] | 7.87 |
| 97 | 1,3,3,8-tetramethyl-5-[[(1R)-1-[3- (1,1-difluoro-2-hydroxy-ethyl)-2- methyl- phenyl]ethyl]amino]pyrrolo[3,2- g]phthalazin-2-one | 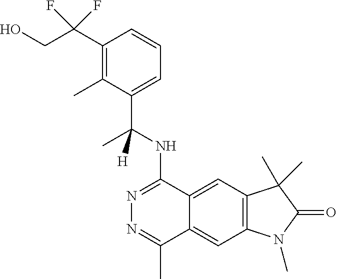 [SMILES: Cc1c([C@@H](C)Nc2nnc(C)c3cc4c(cc23)C(C)(C)C(=O)N4C)cccc1C(F)(F)CO] | 7.88 |
| 98 | 1,3,3,8-tetramethyl-5-[[(1R)-1-[3- (1,1-difluoro-2-hydroxy-2-methyl- propyl)-2-fluoro- phenyl]ethyl]amino]pyrrolo[3,2- g]phthalazin-2-one | 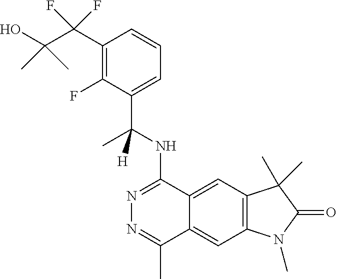 [SMILES: Cc1nnc(N[C@H](C)c2cccc(C(F)(F)C(C)(C)O)c2F)c2cc3c(cc12)N(C)C(=O)C3(C)C] | 7.92 |
| 99 | 1,3,3,8-tetramethyl-5-[[(1R)-1-[3- (1,1-difluoro-2-hydroxy-ethyl)-2- fluoro- phenyl]ethyl]amino]pyrrolo[3,2- g]phthalazin-2-one | 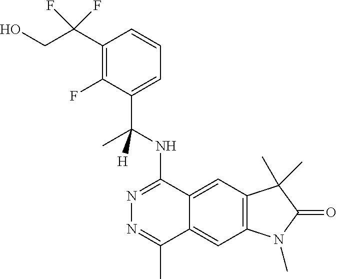 [SMILES: Cc1nnc(N[C@H](C)c2cccc(C(F)(F)CO)c2F)c2cc3c(cc12)N(C)C(=O)C3(C)C] | 8.09 |
| 100 | 3-tert-butyl-1,8-dimethyl-5-[[(1R)- 1-[3-(1,1-difluoro-2-hydroxy- ethyl)-2-methyl- phenyl]ethyl]amino]imidazo[4,5- g]phthalazin-2-one | 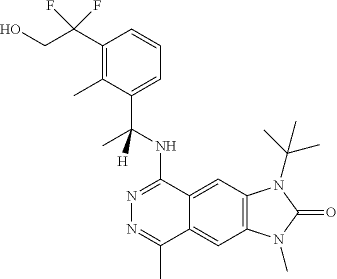 [SMILES: Cc1c([C@@H](C)Nc2nnc(C)c3cc4c(cc23)n(C(C)(C)C)c(=O)n4C)cccc1C(F)(F)CO] | 8.00 |
| 101 | 3-methoxy-1,3,5-trimethyl-8- [[(1R)-1-[3-(1,1-difluoro-2- hydroxy- ethyl)phenyl]ethyl]amino]pyrrolo [2,3-g]phthalazin-2-one | 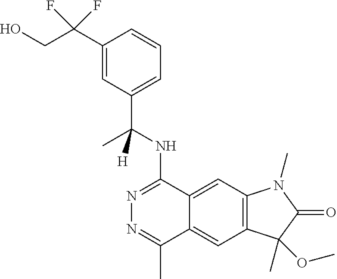 [SMILES: COC1(C)C(=O)N(C)c2cc3c(N[C@H](C)c4cccc(C(F)(F)CO)c4)nnc(C)c3cc21] | 8.00 |
| 102 | 3-methoxy-1,3,5-trimethyl-8- [[(1R)-1-[3-(1,1-difluoro-2- hydroxy-2-methyl-propyl)-2- methyl- phenyl]ethyl]amino]pyrrolo[2,3- g]phthalazin-2-one | 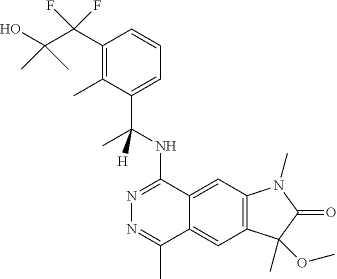 [SMILES: COC1(C)C(=O)N(C)c2cc3c(N[C@H](C)c4cccc(C(F)(F)C(C)(C)O)c4C)nnc(C)c3cc21] | 8.08 |
| 103 | 3-tert-butyl-1,8-dimethyl-5-[[(1R)- 1-[3-(1,1-difluoro-2-hydroxy-2- methyl- propyl)phenyl]ethyl]amino]imidazo [4,5-g]phthalazin-2-one | 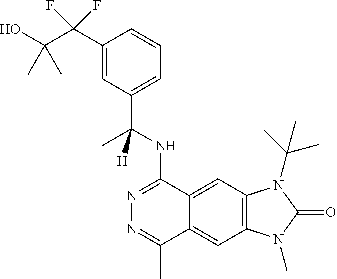 [SMILES: Cc1nnc(N[C@H](C)c2cccc(C(F)(F)C(C)(C)O)c2)c2cc3c(cc12)n(C)c(=O)n3C(C)(C)C] | 8.10 |
| 104 | 1,5-dimethyl-8-[[(1R)-1-[3-(1,1- difluoro-2-hydroxy-2-methyl- propyl)-2-methyl- phenyl]ethyl]amino]spiro[pyrrolo[ 2,3-g]phthalazine-3,4′- tetrahydropyran]-2-one | 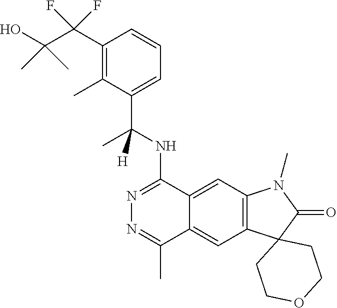 [SMILES: Cc1c([C@@H](C)Nc2nnc(C)c3cc4c(cc23)N(C)C(=O)C42CCOCC2)cccc1C(F)(F)C(C)(C)O] | 7.61 |
| 105 | 8-[[(1R)-1-[3-(1,1-difluoro-2- hydroxy-ethyl)-2-methyl- phenyl]ethyl]amino]-3- (methoxymethyl)-1,3,5-trimethyl- pyrrolo[3,2-g]phthalazin-2-one | 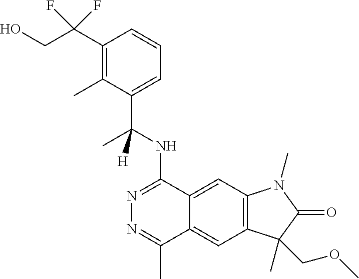 [SMILES: COCC1(C)C(=O)N(C)c2cc3c(N[C@H](C)c4cccc(C(F)(F)CO)c4C)nnc(C)c3cc21] | 7.97 |
| 106 | 8-[[(1R)-1-[3-(1,1-difluoro-2- hydroxy- ethyl)phenyl]ethyl]amino]-3- (methoxymethyl)-1,3,5-trimethyl- pyrrolo[3,2-g]phthalazin-2-one | 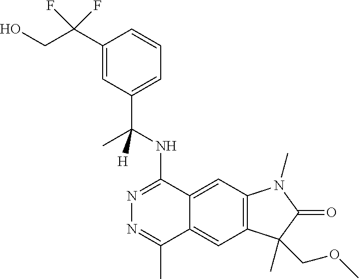 [SMILES: COCC1(C)C(=O)N(C)c2cc3c(N[C@H](C)c4cccc(C(F)(F)CO)c4)nnc(C)c3cc21] | 7.92 |
| 107 | 1,3,3,5-tetramethyl-8-[[(1R)-1-[3- (1,1-difluoro-2-hydroxy-ethyl)-5- fluoro- phenyl]ethyl]amino]pyrrolo[2,3- g]phthalazin-2-one | 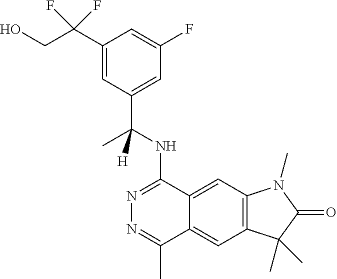 [SMILES: Cc1nnc(N[C@H](C)c2cc(F)cc(C(F)(F)CO)c2)c2cc3c(cc12)C(C)(C)C(=O)N3C] | 7.73 |
| 108 | 1,3,3,5-tetramethyl-8-[[(1R)-1-[3- (1,1-difluoro-2-hydroxy-2-methyl- propyl)-2-methyl- phenyl]ethyl]amino]pyrrolo[2,3- g]phthalazin-2-one | 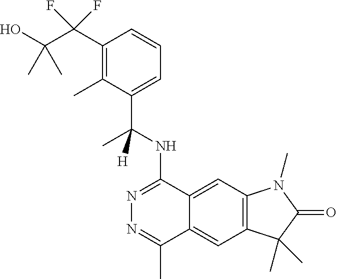 [SMILES: Cc1c([C@@H](C)Nc2nnc(C)c3cc4c(cc23)N(C)C(=O)C4(C)C)cccc1C(F)(F)C(C)(C)O] | 7.98 |
| 109 | 1,3,3,5-tetramethyl-8-[[(1R)-1-[3- (1,1-difluoro-2-hydroxy-ethyl)-2- fluoro- phenyl]ethyl]amino]pyrrolo[2,3- g]phthalazin-2-one | 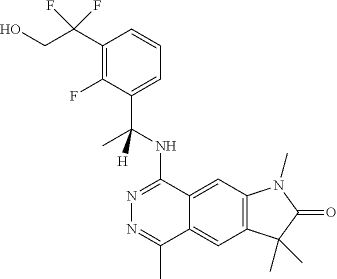 [SMILES: Cc1nnc(N[C@H](C)c2cccc(C(F)(F)CO)c2F)c2cc3c(cc12)C(C)(C)C(=O)N3C] | 8.15 |
| 110 | 1-isopropyl-3,3,5-trimethyl-8- [[(1R)-1-[3-(1,1-difluoro-2- hydroxy-ethyl)-2-fluoro- phenyl]ethyl]amino]pyrrolo[2,3- g]phthalazin-2-one | 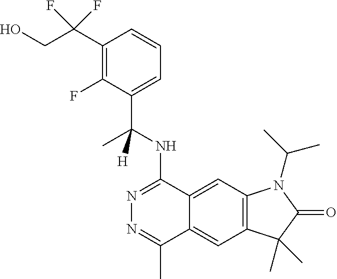 [SMILES: Cc1nnc(N[C@H](C)c2cccc(C(F)(F)CO)c2F)c2cc3c(cc12)C(C)(C)C(=O)N3C(C)C] | 8.15 |
| 111 | 1-isopropyl-3,3,5-trimethyl-8- [[(1R)-1-[3-(1,1-difluoro-2- hydroxy-2-methyl-propyl)-2- fluoro- phenyl]ethyl]amino]pyrrolo[2,3- g]phthalazin-2-one | 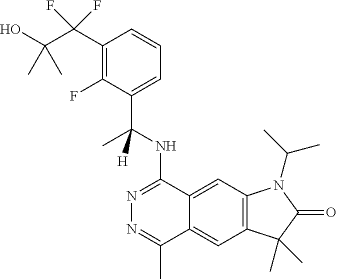 [SMILES: Cc1nnc(N[C@H](C)c2cccc(C(F)(F)C(C)(C)O)c2F)c2cc3c(cc12)C(C)(C)C(=O)N3C(C)C] | 8.13 |
| 112 | 1-cyclopropyl-5-methyl-8-[[(1R)- 1-[3-(1,1-difluoro-2-hydroxy- ethyl)phenyl]ethyl]amino]spiro [pyrrolo[2,3-g]phthalazine-3,4′- tetrahydropyran]-2-one | 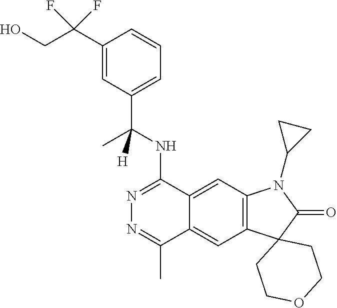 [SMILES: Cc1nnc(N[C@H](C)c2cccc(C(F)(F)CO)c2)c2cc3c(cc12)C1(CCOCC1)C(=O)N3C1CC1] | 7.96 |
| 113 | 8-[[(1R)-1-[3-(1,1-difluoro-2- hydroxy-2-methyl-propyl)-2- methyl-phenyl]ethyl]amino]-3- (methoxymethyl)-1,3,5-trimethyl- pyrrolo[3,2-g]phthalazin-2-one | 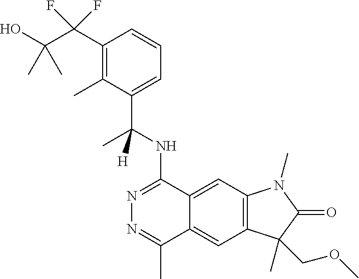 [SMILES: COCC1(C)C(=O)N(C)c2cc3c(N[C@H](C)c4cccc(C(F)(F)C(C)(C)O)c4C)nnc(C)c3cc21] | 7.87 |
| 114 | 1-cyclopropyl-5-methyl-8-[[(1R)- 1-[3-(1, 1-difluoro-2-hydroxy- ethyl)-2-fluoro- phenyl]ethyl]amino]spiro[pyrrolo [2,3-g]phthalazine-3,4′- tetrahydropyran]-2-one | 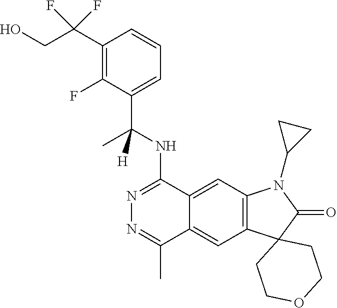 [SMILES: Cc1nnc(N[C@H](C)c2cccc(C(F)(F)CO)c2F)c2cc3c(cc12)C1(CCOCC1)C(=O)N3C1CC1] | 8.04 |
| 115 | 1,8-dimethyl-5-[[(1R)-1-[3-(1,1- difluoro-2-hydroxy-2-methyl- propyl)-2-fluoro- phenyl]ethyl]amino]spiro[pyrrolo[ 3,2-g]phthalazine-3,4′- tetrahydropyran]-2-one | 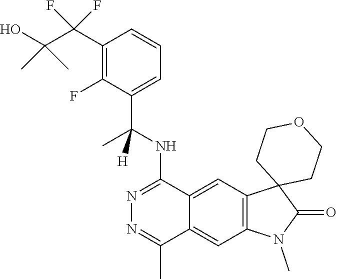 [SMILES: Cc1nnc(N[C@H](C)c2cccc(C(F)(F)C(C)(C)O)c2F)c2cc3c(cc12)N(C)C(=O)C31CCOCC1] | 8.18 |
| 116 | 1,8-dimethyl-5-[[(1R)-1-[3-(1,1- difluoro-2-hydroxy-ethyl)-2- fluoro- phenyl]ethyl]amino]spiro[pyrrolo [3,2-g]phthalazine-3,4′- tetrahydropyran]-2-one | 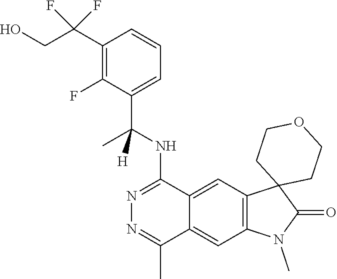 [SMILES: Cc1nnc(N[C@H](C)c2cccc(C(F)(F)CO)c2F)c2cc3c(cc12)N(C)C(=O)C31CCOCC1] | 8.18 |
| 117 | 1-cyclopropyl-3-methoxy-3,5- dimethyl-8-[[(1R)-1-[3-(1,1- difluoro-2-hydroxy-2-methyl- propyl)phenyl]ethyl]amino]pyrrolo [2,3-g]phthalazin-2-one | 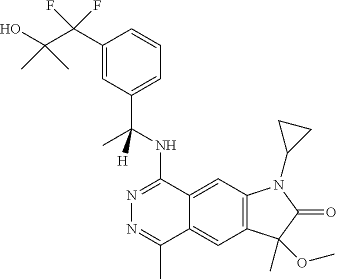 [SMILES: COC1(C)C(=O)N(C2CC2)c2cc3c(N[C@H](C)c4cccc(C(F)(F)C(C)(C)O)c4)nnc(C)c3cc21] | 8.02 |
| 118 | 1,3,3,5-tetramethyl-8-[[(1R)-1-[3- (2-amino-1,1-difluoro-ethyl)-2- fluoro- phenyl]ethyl]amino]pyrrolo[2,3- g]phthalazin-2-one | 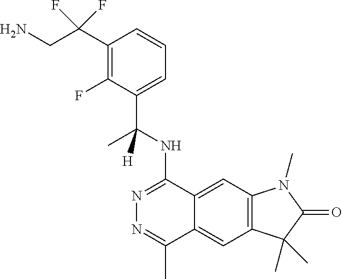 [SMILES: Cc1nnc(N[C@H](C)c2cccc(C(F)(F)CN)c2F)c2cc3c(cc12)C(C)(C)C(=O)N3C] | 7.94 |
| 119 | 1-cyclopropyl-5-methyl-8-[[(1R)- 1-[3-(1,1-difluoro-2-hydroxy- ethyl)-2-methyl- phenyl]ethyl]amino]spiro[pyrrolo [2,3-g]phthalazine-3,4′- tetrahydropyran]-2-one | 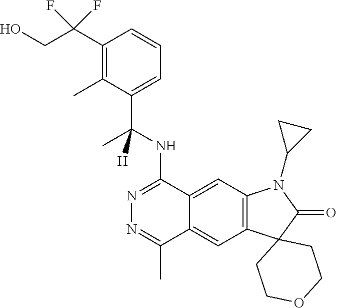 [SMILES: Cc1c([C@@H](C)Nc2nnc(C)c3cc4c(cc23)N(C2CC2)C(=O)C42CCOCC2)cccc1C(F)(F)CO] | 8.13 |
| 120 | 1-cyclopropyl-5-methyl-8-[[(1R)- 1-[3-(1,1-difluoro-2-hydroxy-2- methyl- propyl)phenyl]ethyl]amino]spiro [pyrrolo[2,3-g]phthalazine-3,4′- tetrahydropyran]-2-one | 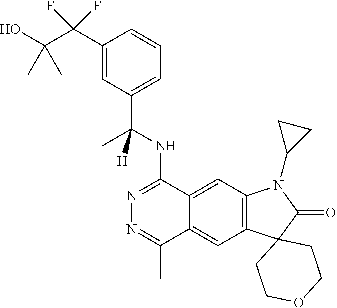 [SMILES: Cc1nnc(N[C@H](C)c2cccc(C(F)(F)C(C)(C)O)c2)c2cc3c(cc12)C1(CCOCC1)C(=O)N3C1CC1] | 7.98 |
| 121 | 1-cyclopropyl-5-methyl-8-[[(1R)- 1-[3-(1,1-difluoro-2-hydroxy-2- methyl-propyl)-2-fluoro- phenyl]ethyl]amino]spiro[pyrrolo [2,3-g]phthalazine-3,4′- tetrahydropyran]-2-one | 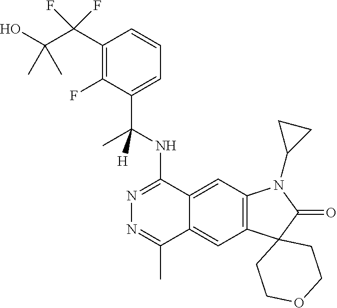 [SMILES: Cc1nnc(N[C@H](C)c2cccc(C(F)(F)C(C)(C)O)c2F)c2cc3c(cc12)C1(CCOCC1)C(=O)N3C1CC1] | 8.20 |
| 122 | 1-cyclopropyl-3-methoxy-3,5- dimethyl-8-[[(1R)-1-[3-(1,1- difluoro-2-hydroxy-ethyl)-2- fluoro- phenyl]ethyl]amino]pyrrolo[2,3- g]phthalazin-2-one | 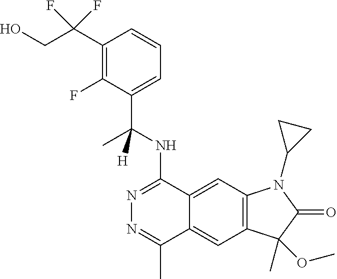 [SMILES: COC1(C)C(=O)N(C2CC2)c2cc3c(N[C@H](C)c4cccc(C(F)(F)CO)c4F)nnc(C)c3cc21] | 8.24 |
| 123 | 1-cyclopropyl-3,3,5-trimethyl-8- [[(1R)-1-[3-(1,1-difluoro-2- hydroxy-ethyl)-2-fluoro- phenyl]ethyl]amino]pyrrolo[2,3- g]phthalazin-2-one | 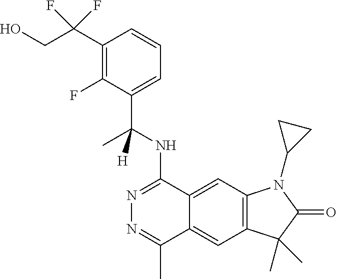 [SMILES: Cc1nnc(N[C@H](C)c2cccc(C(F)(F)CO)c2F)c2cc3c(cc12)C(C)(C)C(=O)N3C1CC1] | 8.06 |
| 124 | 1-isopropyl-3,3,5-trimethyl-8- [[(1R)-1-[3-(1,1-difluoro-2- hydroxy-2-methyl-propyl)-2- methyl- phenyl]ethyl]amino]pyrrolo[2,3- g]phthalazin-2-one | 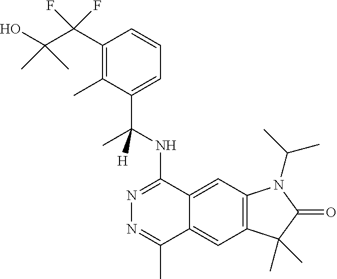 [SMILES: Cc1c([C@@H](C)Nc2nnc(C)c3cc4c(cc23)N(C(C)C)C(=O)C4(C)C)cccc1C(F)(F)C(C)(C)O] | 8.21 |
| 125 | 1,8-dimethyl-5-[[(1R)-1-[3-(1,1- difluoro-2-hydroxy-2-methyl- propyl)phenyl]ethyl]amino]spiro [pyrrolo[3,2-g]phthalazine-3,4′- tetrahydropyran]-2-one | 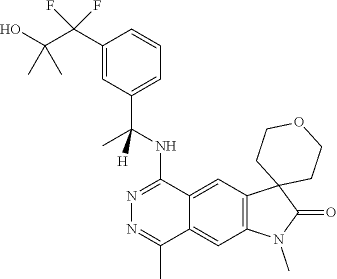 [SMILES: Cc1nnc(N[C@H](C)c2cccc(C(F)(F)C(C)(C)O)c2)c2cc3c(cc12)N(C)C(=O)C31CCOCC1] | 8.18 |
| 126 | 3-ethoxy-1,3,5-trimethyl-8-[[(1R)- 1-[3-(1, 1-difluoro-2-hydroxy-2- methyl-propyl)-2-methyl- phenyl]ethyl]amino]pyrrolo[2,3- g]phthalazin-2-one | 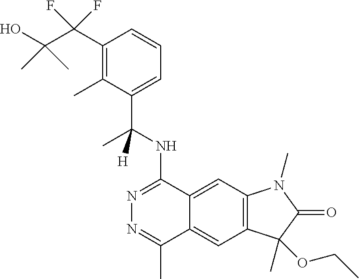 [SMILES: CCOC1(C)C(=O)N(C)c2cc3c(N[C@H](C)c4cccc(C(F)(F)C(C)(C)O)c4C)nnc(C)c3cc21] | 8.04 |
| 127 | 1-cyclopropyl-3,3,5-trimethyl-8- [[(1R)-1-[3-(1,1-difluoro-2- hydroxy-2-methyl-propyl)-2- methyl- phenyl]ethyl]amino]pyrrolo[2,3- g]phthalazin-2-one | 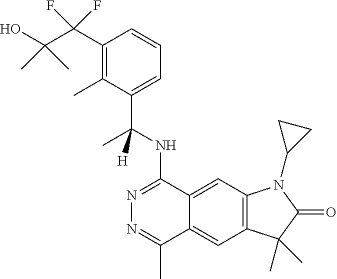 [SMILES: Cc1c([C@@H](C)Nc2nnc(C)c3cc4c(cc23)N(C2CC2)C(=O)C4(C)C)cccc1C(F)(F)C(C)(C)O] | 7.89 |
| 128 | 1,8-dimethyl-5-[[(1R)-1-[3-(1,1- difluoro-2-hydroxy- ethyl)phenyl]ethyl]amino]spiro [pyrrolo[3,2-g]phthalazine-3,4′- tetrahydropyran]-2-one | 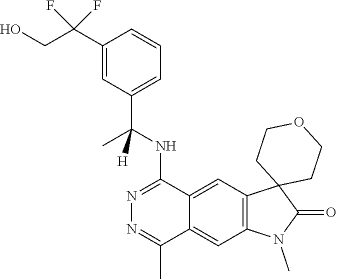 [SMILES: Cc1nnc(N[C@H](C)c2cccc(C(F)(F)CO)c2)c2cc3c(cc12)N(C)C(=O)C31CCOCC1] | 8.20 |
| 129 | 1,8-dimethyl-5-[[(1R)-1-[3-(1,1- difluoro-2-hydroxy-ethyl)-2- methyl- phenyl]ethyl]amino]spiro[pyrrolo [3,2-g]phthalazine-3,4′- tetrahydropyran]-2-one | 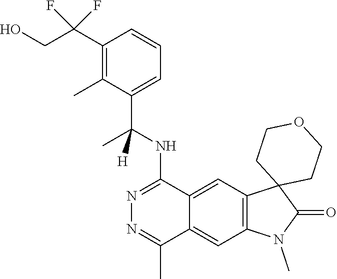 [SMILES: Cc1c([C@@H](C)Nc2nnc(C)c3cc4c(cc23)C2(CCOCC2)C(=O)N4C)cccc1C(F)(F)CO] | 8.18 |
| 130 | 3-ethoxy-1,3,5-trimethyl-8-[[(1R)- 1-[3-(1,1-difluoro-2-hydroxy-2- methyl- propyl)phenyl]ethyl]amino]pyrrolo [2,3-g]phthalazin-2-one | 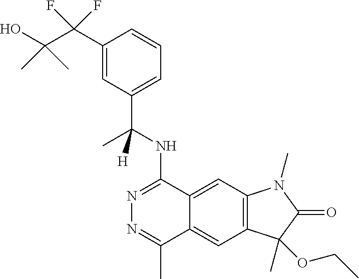 [SMILES: CCOC1(C)C(=O)N(C)c2cc3c(N[C@H](C)c4cccc(C(F)(F)C(C)(C)O)c4)nnc(C)c3cc21] | 8.14 |
| 131 | 1,8-dimethyl-5-[[(1R)-1-[3-(1,1- difluoro-2-hydroxy-2-methyl- propyl)-2-methyl- phenyl]ethyl]amino]spiro[pyrrolo [3,2-g]phthalazine-3,4′- tetrahydropyran]-2-one | 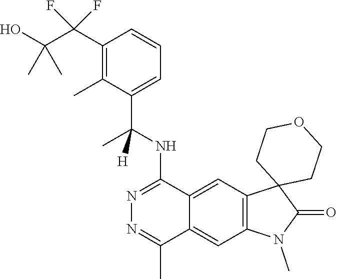 [SMILES: Cc1c([C@@H](C)Nc2nnc(C)c3cc4c(cc23)C2(CCOCC2)C(=O)N4C)cccc1C(F)(F)C(C)(C)O] | 8.10 |
| 132 | 3-ethoxy-1,3,5-trimethyl-8-[[(1R)- 1-[3-(1, 1-difluoro-2-hydroxy-2- methyl-propyl)-2-fluoro- phenyl]ethyl]amino]pyrrolo[2,3- g]phthalazin-2-one | 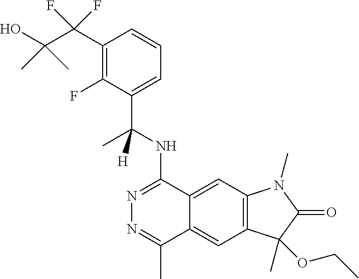 [SMILES: CCOC1(C)C(=O)N(C)c2cc3c(N[C@H](C)c4cccc(C(F)(F)C(C)(C)O)c4F)nnc(C)c3cc21] | 8.10 |
| 133 | 3-ethoxy-1,3,5-trimethyl-8-[[(1R)- 1-[3-(1, 1-difluoro-2-hydroxy- ethyl)phenyl]ethyl]amino]pyrrolo [2,3-g]phthalazin-2-one | 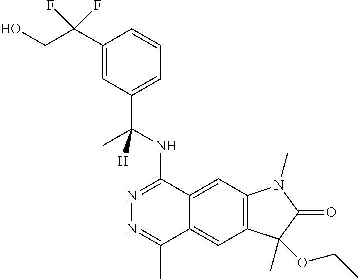 [SMILES: CCOC1(C)C(=O)N(C)c2cc3c(N[C@H](C)c4cccc(C(F)(F)CO)c4)nnc(C)c3cc21] | 8.07 |
| 134 | 3-ethoxy-1,3,5-trimethyl-8-[[(1R)- 1-[3-(1, 1-difluoro-2-hydroxy- ethyl)-2-methyl- phenyl]ethyl]amino]pyrrolo[2,3- g]phthalazin-2-one | 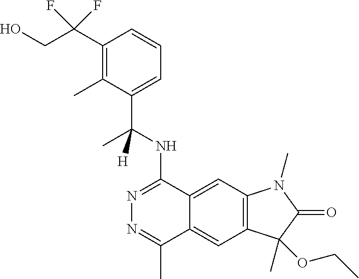 [SMILES: CCOC1(C)C(=O)N(C)c2cc3c(N[C@H](C)c4cccc(C(F)(F)CO)c4C)nnc(C)c3cc21] | 8.21 |
| 135 | 3-ethoxy-1,3,5-trimethyl-8-[[(1R)- 1-[3-(1,1-difluoro-2-hydroxy- ethyl)-2-fluoro- phenyl]ethyl]amino]pyrrolo[2,3- g]phthalazin-2-one | 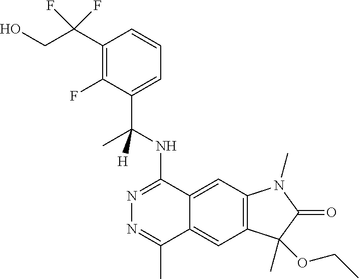 [SMILES: CCOC1(C)C(=O)N(C)c2cc3c(N[C@H](C)c4cccc(C(F)(F)CO)c4F)nnc(C)c3cc21] | 8.00 |
| 136 | 1-cyclopropyl-8-[[(1R)-1-[3-(1,1- difluoro-2-hydroxy-2-methyl- propyl)-2-fluoro- phenyl]ethyl]amino]-3- (methoxymethyl)-3,5-dimethyl- pyrrolo[3,2-g]phthalazin-2-one | 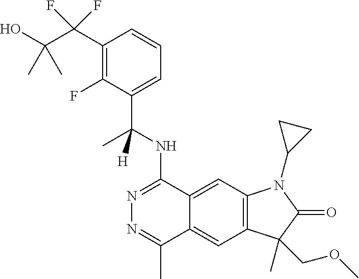 [SMILES: COCC1(C)C(=O)N(C2CC2)c2cc3c(N[C@H](C)c4cccc(C(F)(F)C(C)(C)O)c4F)nnc(C)c3cc21] | 7.85 |
| 137 | 1-cyclopropyl-8-[[(1R)-1-[3-(1,1- difluoro-2-hydroxy-2-methyl- propyl)-2-methyl- phenyl]ethyl]amino]-3- (methoxymethyl)-3,5-dimethyl- pyrrolo[3,2-g]phthalazin-2-one | 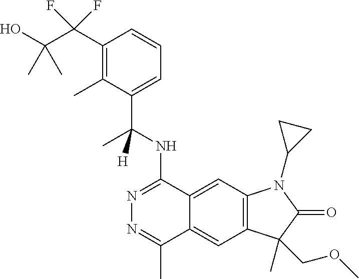 [SMILES: COCC1(C)C(=O)N(C2CC2)c2cc3c(N[C@H](C)c4cccc(C(F)(F)C(C)(C)O)c4C)nnc(C)c3cc21] | 7.80 |
| 138 | 1-cyclopropyl-8-[[(1R)-1-[3-(1,1- difluoro-2-hydroxy-ethyl)-2- fluoro-phenyl]ethyl]amino]-3- (methoxymethyl)-3,5-dimethyl- pyrrolo[3,2-g]phthalazin-2-one | 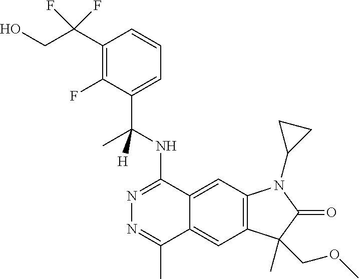 [SMILES: COCC1(C)C(=O)N(C2CC2)c2cc3c(N[C@H](C)c4cccc(C(F)(F)CO)c4F)nnc(C)c3cc21] | 8.18 |
| 139 | 1-cyclopropyl-3-methoxy-3,5- dimethyl-8-[[(1R)-1-[3-(1,1- difluoro-2-hydroxy-2-methyl- propyl)-2-fluoro- phenyl]ethyl]amino]pyrrolo[2,3- g]phthalazin-2-one | 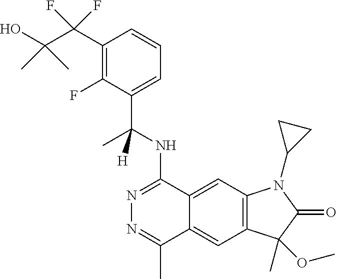 [SMILES: COC1(C)C(=O)N(C2CC2)c2cc3c(N[C@H](C)c4cccc(C(F)(F)C(C)(C)O)c4F)nnc(C)c3cc21] | 7.96 |
| 140 | 1-cyclopropyl-3-methoxy-3,5- dimethyl-8-[[(1R)-1-[3-(1,1- difluoro-2-hydroxy-2-methyl- propyl)-2-methyl- phenyl]ethyl]amino]pyrrolo[2,3- g]phthalazin-2-one | 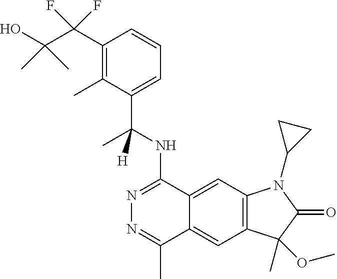 [SMILES: COC1(C)C(=O)N(C2CC2)c2cc3c(N[C@H](C)c4cccc(C(F)(F)C(C)(C)O)c4C)nnc(C)c3cc21] | 7.81 |
| 141 | 3-methoxy-1,3,5-trimethyl-8- [[(1R)-1-[3-(1,1-difluoro-2- hydroxy-ethyl)-2,5-difluoro- phenyl]ethyl]amino]pyrrolo[2,3- g]phthalazin-2-one | 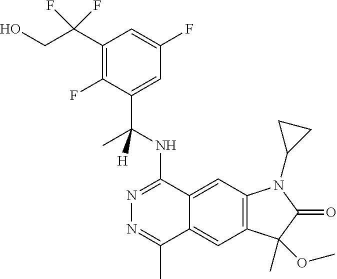 [SMILES: COC1(C)C(=O)N(C2CC2)c2cc3c(N[C@H](C)c4cc(F)cc(C(F)(F)CO)c4F)nnc(C)c3cc21] | 7.70 |
| 142 | 1-cyclopropyl-3-methoxy-3,5- dimethyl-8-[[(1R)-1-[3-(1,1- difluoro-2-hydroxy- ethyl)phenyl]ethyl]amino]pyrrolo [2,3-g]phthalazin-2-one | 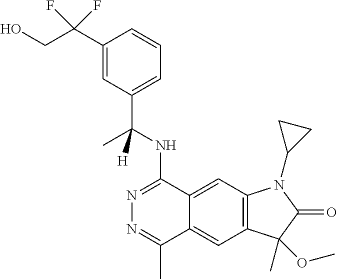 [SMILES: COC1(C)C(=O)N(C2CC2)c2cc3c(N[C@H](C)c4cccc(C(F)(F)CO)c4)nnc(C)c3cc21] | 7.89 |
| 143 | 1-cyclopropyl-3-methoxy-3,5- dimethyl-8-[[(1R)-1-[3-(1,1- difluoro-2-hydroxy-ethyl)-2- methyl- phenyl]ethyl]amino]pyrrolo[2,3- g]phthalazin-2-one | 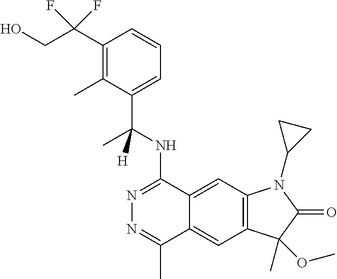 [SMILES: COC1(C)C(=O)N(C2CC2)c2cc3c(N[C@H](C)c4cccc(C(F)(F)CO)c4C)nnc(C)c3cc21] | 7.96 |
| 144 | 1,8-dimethyl-3-tetrahydrofuran-3- yl-5-[[(1R)-1-[3- (trifluoromethyl)phenyl]ethyl]amino] imidazo[4,5-g]phthalazin-2-one | 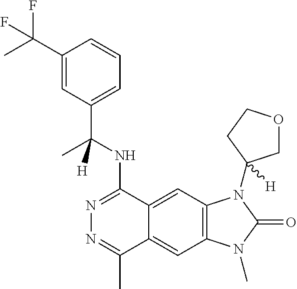 [SMILES: Cc1nnc(N[C@H](C)c2cccc(C(C)(F)F)c2)c2cc3c(cc12)n(C)c(=O)n3C1CCOC1] | 7.93 |
| 145 | 1,8-dimethyl-5-[[(1R)-1-[3- (trifluoromethyl)phenyl]ethyl]amino]- 3H-imidazo[4,5-g]phthalazin- 2-one | 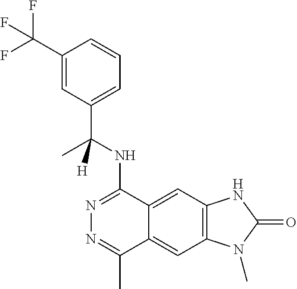 [SMILES: Cc1nnc(N[C@H](C)c2cccc(C(F)(F)F)c2)c2cc3[nH]c(=O)n(C)c3cc12] | 7.18 |
| 146 | 5-[[(1R)-1-[3-(difluoromethyl)-2- fluoro-phenyl]ethyl]amino]-1,8- dimethyl-3-[(3S)-tetrahydrofuran- 3-yl]imidazo[4,5-g]phthalazin-2- one | 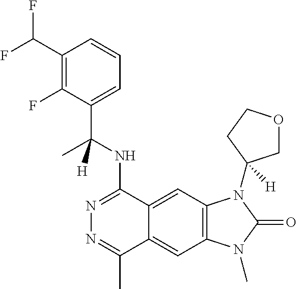 [SMILES: Cc1nnc(N[C@H](C)c2cccc(C(F)F)c2F)c2cc3c(cc12)n(C)c(=O)n3[C@H]1CCOC1] | 7.91 |
| 147 | 5-[[(1R)-1-[3-(difluoromethyl)-2- fluoro-phenyl]ethyl]amino]-1,8- dimethyl-3-(oxetan-3- yl)imidazo[4,5-g]phthalazin-2-one | 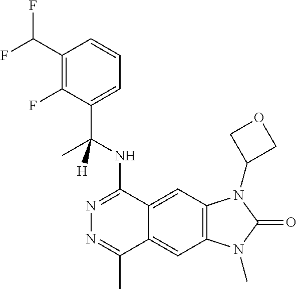 [SMILES: Cc1nnc(N[C@H](C)c2cccc(C(F)F)c2F)c2cc3c(cc12)n(C)c(=O)n3C1COC1] | 7.91 |
| 148 | 2-[1,8-dimethyl-2-oxo-5-[[(1R)-1- [3- (trifluoromethyl)phenyl]ethyl] amino]imidazo[4,5-g]phthalazin-3- yl]acetonitrile | 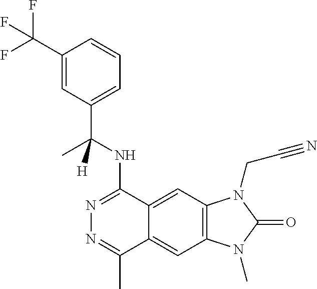 [SMILES: Cc1nnc(N[C@H](C)c2cccc(C(F)(F)F)c2)c2cc3c(cc12)n(C)c(=O)n3CC#N] | 7.67 |
| 149 | 2-[1,8-dimethyl-2-oxo-5-[[(1R)-1-[3- (trifluoromethyl)phenyl]ethyl] amino]imidazo[4,5-g]phthalazin-3- yl]propanenitrile | 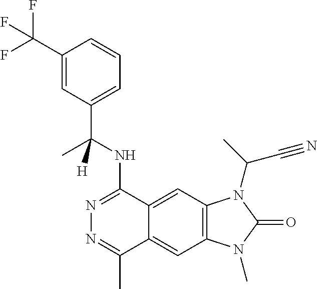 [SMILES: Cc1nnc(N[C@H](C)c2cccc(C(F)(F)F)c2)c2cc3c(cc12)n(C)c(=O)n3C(C)C#N] | 7.99 |
| 150 | 1,3,8-trimethyl-5-[[(1R)-1-[2- methyl-3-(2-methylpyrazol-3- yl)phenyl]ethyl]amino]imidazo[4, 5-g]phthalazin-2-one | 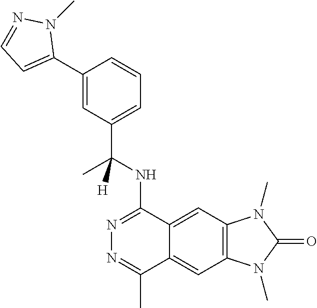 [SMILES: Cc1nnc(N[C@H](C)c2cccc(-c3ccnn3C)c2)c2cc3c(cc12)n(C)c(=O)n3C] | 8.05 |
| 151 | 1,3,8-trimethyl-5-[[(1R)-1-[2- methyl-3-(1-methylpyrazol-3- yl)phenyl]ethyl]amino]imidazo[4, 5-g]phthalazin-2-one | 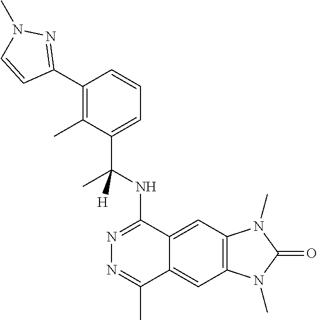 [SMILES: Cc1c(-c2ccn(C)n2)cccc1[C@@H](C)Nc1nnc(C)c2cc3c(cc12)n(C)c(=O)n3C] | 7.96 |
| 152 | 1,3,8-trimethyl-5-[[(1R)-1-[2- methyl-3-[2-methyl-5- (trifluoromethyl)pyrazol-3- yl]phenyl]ethyl]amino]imidazo[4, 5-g]phthalazin-2-one | 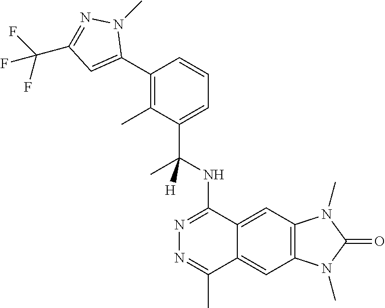 [SMILES: Cc1c(-c2cc(C(F)(F)F)nn2C)cccc1[C@@H](C)Nc1nnc(C)c2cc3c(cc12)n(C)c(=O)n3C] | 7.07 |
| 153 | 1,3,8-trimethyl-5-[[(1R)-1-(2- methyl-3-pyrimidin-5-yl- phenyl)ethyl]amino]imidazo[4,5- g]phthalazin-2-one | 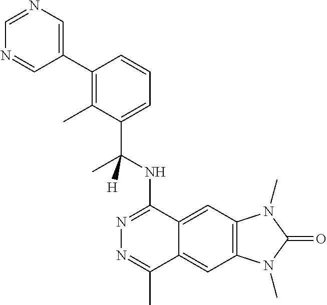 [SMILES: Cc1c(-c2cncnc2)cccc1[C@@H](C)Nc1nnc(C)c2cc3c(cc12)n(C)c(=O)n3C] | 6.97 |
| 154 | 1,3,8-trimethyl-5-[[(1R)-1-(3- cyclopropyl-2-methyl- phenyl)ethyl]amino]imidazo[4,5- g]phthalazin-2-one | 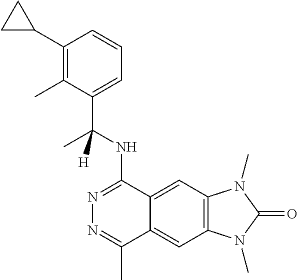 [SMILES: Cc1c(C2CC2)cccc1[C@@H](C)Nc1nnc(C)c2cc3c(cc12)n(C)c(=O)n3C] | 7.80 |
| 155 | 1,3,8-trimethyl-5-[[(1R)-1-[2- methyl-3-(1H-pyrazol-5- yl)phenyl]ethyl]amino]imidazo[4, 5-g]phthalazin-2-one | 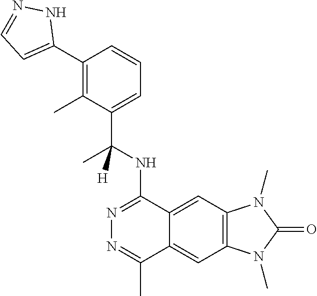 [SMILES: Cc1c(-c2ccn[nH]2)cccc1[C@@H](C)Nc1nnc(C)c2cc3c(cc12)n(C)c(=O)n3C] | 7.51 |
| 156 | 1,3,8-trimethyl-5-[[(1R)-1-[3-(2,5- dimethylpyrazol-3-yl)-2-methyl- phenyl]ethyl]amino]imidazo[4,5- g]phthalazin-2-one | 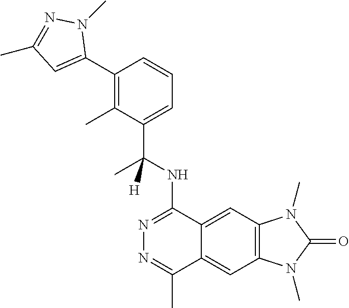 [SMILES: Cc1cc(-c2cccc([C@@H](C)Nc3nnc(C)c4cc5c(cc34)n(C)c(=O)n5C)c2C)n(C)n1] | 7.05 |
| 157 | 5-[[(1R)-1-[3-(3,6-dihydro-2H- pyran-4-yl)-2-methyl- phenyl]ethyl]amino]-1,3,8- trimethyl-imidazo[4,5- g]phthalazin-2-one | 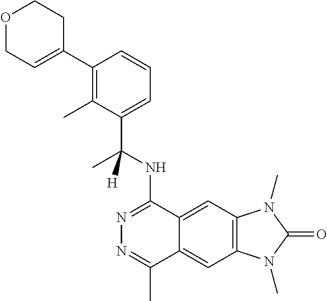 [SMILES: Cc1c(C2=CCOCC2)cccc1[C@@H](C)Nc1nnc(C)c2cc3c(cc12)n(C)c(=O)n3C] | 6.69 |
| 158 | 1,3,8-trimethyl-5-[[(1R)-1-[3-(2,5- dihydro-1H-pyrrol-3-yl)-2-methyl- phenyl]ethyl]amino]imidazo[4,5- g]phthalazin-2-one | 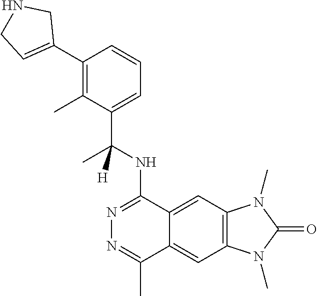 [SMILES: Cc1c(C2=CCNC2)cccc1[C@@H](C)Nc1nnc(C)c2cc3c(cc12)n(C)c(=O)n3C] | 6.92 |
| 159 | 1,3,8-trimethyl-5-[[(1R)-1-(2- methyl-3-thiazol-5-yl- phenyl)ethyl]amino]imidazo[4,5- g]phthalazin-2-one | 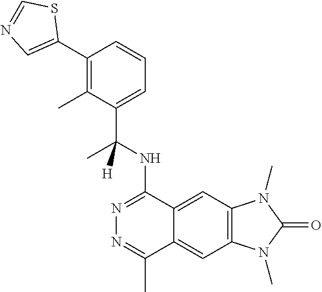 [SMILES: Cc1c(-c2cncs2)cccc1[C@@H](C)Nc1nnc(C)c2cc3c(cc12)n(C)c(=O)n3C] | 7.75 |
| 160 | 1,3,8-trimethyl-5-[[(1R)-1-(2- methyl-3-tetrahydropyran-4-yl- phenyl)ethyl]amino]imidazo[4,5- g]phthalazin-2-one | 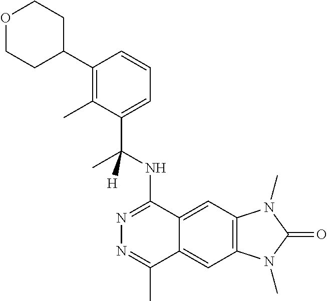 [SMILES: Cc1c(C2CCOCC2)cccc1[C@@H](C)Nc1nnc(C)c2cc3c(cc12)n(C)c(=O)n3C] | 6.57 |
| 161 | 1,3,8-trimethyl-5-[[(1R)-1-(2- methyl-3-pyrrolidin-3-yl- phenyl)ethyl]amino]imidazo[4,5- g]phthalazin-2-one | 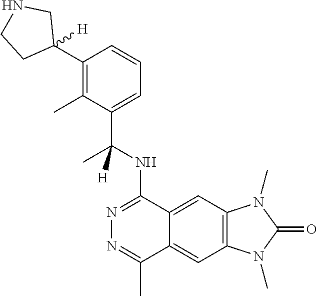 [SMILES: Cc1c(C2CCNC2)cccc1[C@@H](C)Nc1nnc(C)c2cc3c(cc12)n(C)c(=O)n3C] | 6.23 |
| 162 | 1-cyclopropyl-8-[[(1R)-1-[3-(1,1- difluoro-2-hydroxy- ethyl)phenyl]ethyl]amino]-3- ethoxy-3,5-dimethyl-pyrrolo[3,2- g]phthalazin-2-one | 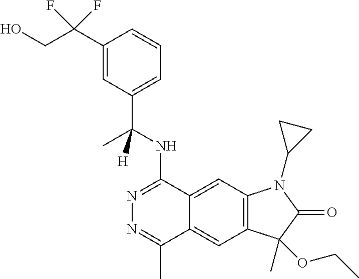 [SMILES: CCOC1(C)C(=O)N(C2CC2)c2cc3c(N[C@H](C)c4cccc(C(F)(F)CO)c4)nnc(C)c3cc21] | 8.04 |
| 163 | 1-cyclopropyl-3-ethoxy-3,5- dimethyl-8-[[(1R)-1-[3-(1,1- difluoro-2-hydroxy-2-methyl- propyl)-2-fluoro- phenyl]ethyl]amino]pyrrolo[2,3- g]phthalazin-2-one | 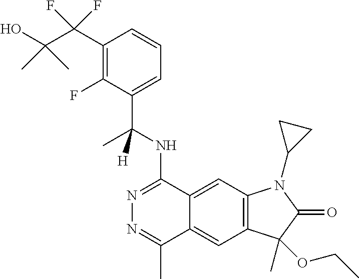 [SMILES: CCOC1(C)C(=O)N(C2CC2)c2cc3c(N[C@H](C)c4cccc(C(F)(F)C(C)(C)O)c4F)nnc(C)c3cc21] | 7.97 |
| 164 | 1-cyclopropyl-3-ethoxy-3,5- dimethyl-8-[[(1R)-1-[3-(1,1- difluoro-2-hydroxy-ethyl)-2- fluoro- phenyl]ethyl]amino]pyrrolo[2,3- g]phthalazin-2-one | 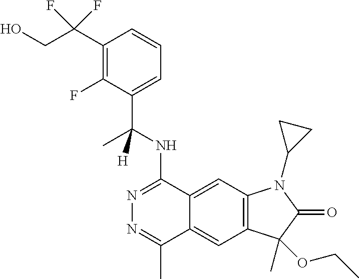 [SMILES: CCOC1(C)C(=O)N(C2CC2)c2cc3c(N[C@H](C)c4cccc(C(F)(F)CO)c4F)nnc(C)c3cc21] | 7.94 |
| 165 | 1-cyclopropyl-3-ethoxy-3,5- dimethyl-8-[[(1R)-1-[3-(1,1- difluoro-2-hydroxy-2-methyl- propyl)phenyl]ethyl]amino]pyrrolo [2,3-g]phthalazin-2-one | 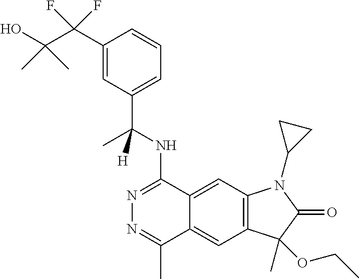 [SMILES: CCOC1(C)C(=O)N(C2CC2)c2cc3c(N[C@H](C)c4cccc(C(F)(F)C(C)(C)O)c4)nnc(C)c3cc21] | 8.00 |
| 166 | 1-cyclopropyl-3-ethoxy-3,5- dimethyl-8-[[(1R)-1-[3-(1,1- difluoro-2-hydroxy-2-methyl- propyl)-2-methyl- phenyl]ethyl]amino]pyrrolo[2,3- g]phthalazin-2-one | 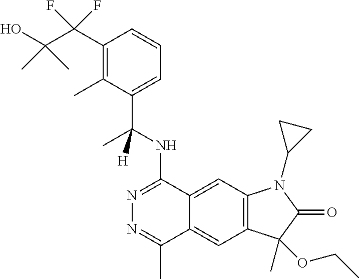 [SMILES: CCOC1(C)C(=O)N(C2CC2)c2cc3c(N[C@H](C)c4cccc(C(F)(F)C(C)(C)O)c4C)nnc(C)c3cc21] | 7.83 |
| 167 | 1-cyclopropyl-3-ethoxy-3,5- dimethyl-8-[[(1R)-1-[3-(1,1- difluoro-2-hydroxy-ethyl)-2- methyl- phenyl]ethyl]amino]pyrrolo[2,3- g]phthalazin-2-one | 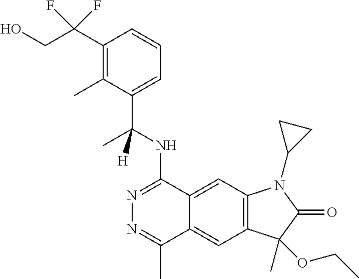 [SMILES: CCOC1(C)C(=O)N(C2CC2)c2cc3c(N[C@H](C)c4cccc(C(F)(F)CO)c4C)nnc(C)c3cc21] | 7.90 |
| 168 | 1-ethyl-3-methoxy-3,5-dimethyl- 8-[[(1R)-1-[3-(1,1-difluoro-2- hydroxy-ethyl)-2-methyl- phenyl]ethyl]amino]pyrrolo[2,3- g]phthalazin-2-one | 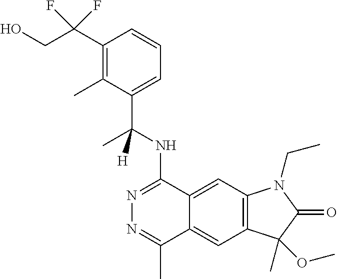 [SMILES: CCN1C(=O)C(C)(OC)c2cc3c(C)nnc(N[C@H](C)c4cccc(C(F)(F)CO)c4C)c3cc21] | 8.05 |
| 169 | 1-ethyl-3-methoxy-3,5-dimethyl- 8-[[(1R)-1-[3-(1,1-difluoro-2- hydroxy-2-methyl-propyl)-2- methyl- phenyl]ethyl]amino]pyrrolo[2,3- g]phthalazin-2-one | 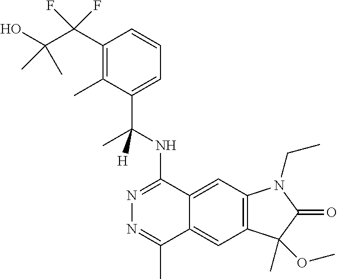 [SMILES: CCN1C(=O)C(C)(OC)c2cc3c(C)nnc(N[C@H](C)c4cccc(C(F)(F)C(C)(C)O)c4C)c3cc21] | 7.99 |
| 170 | 1-ethyl-3-methoxy-3,5-dimethyl- 8-[[(1R)-1-[3-(1,1-difluoro-2- hydroxy-2-methyl-propyl)-2- fluoro- phenyl]ethyl]amino]pyrrolo[2,3- g]phthalazin-2-one | 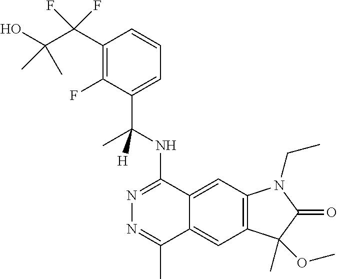 [SMILES: CCN1C(=O)C(C)(OC)c2cc3c(C)nnc(N[C@H](C)c4cccc(C(F)(F)C(C)(C)O)c4F)c3cc21] | 8.04 |
| 171 | 1-ethyl-3-methoxy-3,5-dimethyl- 8-[[(1R)-1-[3-(1,1-difluoro-2- hydroxy- ethyl)phenyl]ethyl]amino]pyrrolo [2,3-g]phthalazin-2-one | 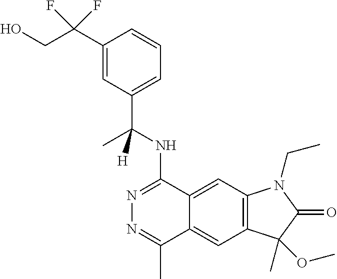 [SMILES: CCN1C(=O)C(C)(OC)c2cc3c(C)nnc(N[C@H](C)c4cccc(C(F)(F)CO)c4)c3cc21] | 8.01 |
| 172 | 1-ethyl-3-methoxy-3,5-dimethyl- 8-[[(1R)-1-[3-(1,1-difluoro-2- hydroxy-ethyl)-2-fluoro- phenyl]ethyl]amino]pyrrolo[2,3- g]phthalazin-2-one | 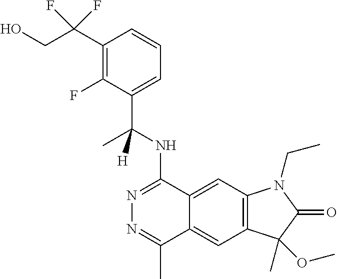 [SMILES: CCN1C(=O)C(C)(OC)c2cc3c(C)nnc(N[C@H](C)c4cccc(C(F)(F)CO)c4F)c3cc21] | 8.01 |
| 173 | 1-ethyl-3-methoxy-3,5-dimethyl- 8-[[(1R)-1-[3-(1,1-difluoro-2- hydroxy-2-methyl- propyl)phenyl]ethyl]amino]pyrrolo [2,3-g]phthalazin-2-one | 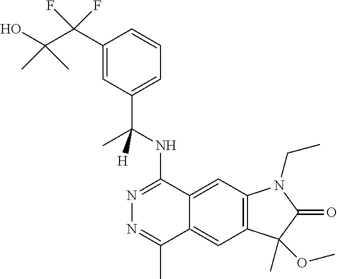 [SMILES: CCN1C(=O)C(C)(OC)c2cc3c(C)nnc(N[C@H](C)c4cccc(C(F)(F)C(C)(C)O)c4)c3cc21] | 7.93 |
| 174 | 3-methoxy-1,3,8-trimethyl-5- [[(1R)-1-[3-(1,1-difluoro-2- hydroxy-2-methyl-propyl)-2- methyl- phenyl]ethyl]amino]pyrrolo[3,2- g]phthalazin-2-one | 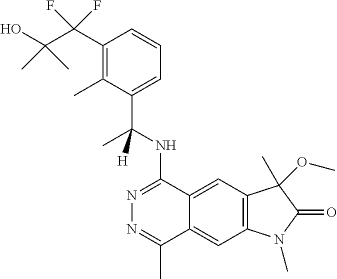 [SMILES: COC1(C)C(=O)N(C)c2cc3c(C)nnc(N[C@H](C)c4cccc(C(F)(F)C(C)(C)O)c4C)c3cc21] | 7.80 |
| 175 | 3-methoxy-1,3,5-trimethyl-8- [[(1R)-1-[3-(1,1-difluoro-2- hydroxy-2-methyl-propyl)-5- fluoro- phenyl]ethyl]amino]pyrrolo[2,3- g]phthalazin-2-one | 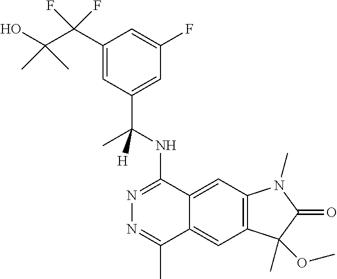 [SMILES: COC1(C)C(=O)N(C)c2cc3c(N[C@H](C)c4cc(F)cc(C(F)(F)C(C)(C)O)c4)nnc(C)c3cc21] | 7.84 |
| 176 | 3-methoxy-1,3,8-trimethyl-5- [[(1R)-1-[3-(1,1-difluoro-2- hydroxy-ethyl)-2-fluoro- phenyl]ethyl]amino]pyrrolo[3,2- g]phthalazin-2-one | 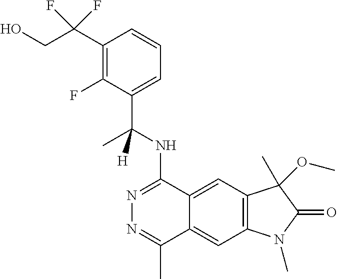 [SMILES: COC1(C)C(=O)N(C)c2cc3c(C)nnc(N[C@H](C)c4cccc(C(F)(F)CO)c4F)c3cc21] | 7.97 |
| 177 | 3-methoxy-1,3,8-trimethyl-5- [[(1R)-1-[3-(1,1-difluoro-2- hydroxy-ethyl)-2-methyl- phenyl]ethyl]amino]pyrrolo[3,2- g]phthalazin-2-one | 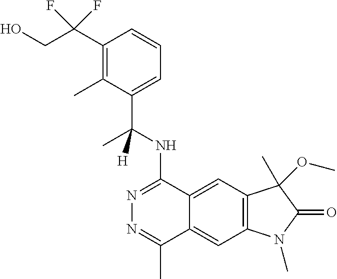 [SMILES: COC1(C)C(=O)N(C)c2cc3c(C)nnc(N[C@H](C)c4cccc(C(F)(F)CO)c4C)c3cc21] | 8.01 |
| 178 | 3-methoxy-1,3,8-trimethyl-5- [[(1R)-1-[3-(1,1-difluoro-2- hydroxy- ethyl)phenyl]ethyl]amino]pyrrolo [3,2-g]phthalazin-2-one | 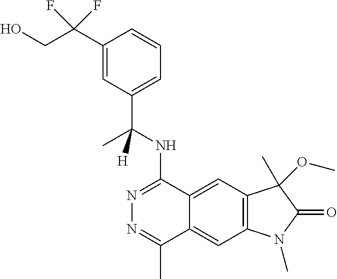 [SMILES: COC1(C)C(=O)N(C)c2cc3c(C)nnc(N[C@H](C)c4cccc(C(F)(F)CO)c4)c3cc21] | 7.78 |
| 179 | 3-methoxy-1,3,8-trimethyl-5- [[(1R)-1-[3-(1,1-difluoro-2- hydroxy-2-methyl-propyl)-2- fluoro- phenyl]ethyl]amino]pyrrolo[3,2- g]phthalazin-2-one | 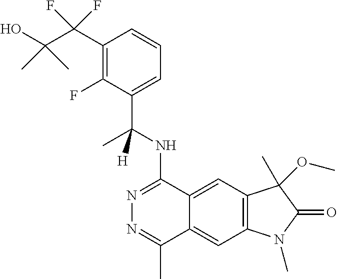 [SMILES: COC1(C)C(=O)N(C)c2cc3c(C)nnc(N[C@H](C)c4cccc(C(F)(F)C(C)(C)O)c4F)c3cc21] | 7.99 |
| 180 | 3-methoxy-1,3,8-trimethyl-5- [[(1R)-1-[3-(1,1-difluoro-2- hydroxy-2-methyl- propyl)phenyl]ethyl]amino]pyrrolo [3,2-g]phthalazin-2-one | 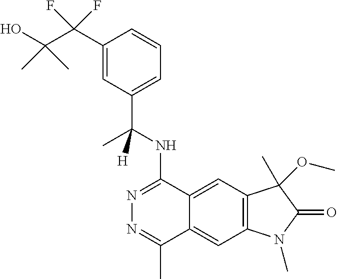 [SMILES: COC1(C)C(=O)N(C)c2cc3c(C)nnc(N[C@H](C)c4cccc(C(F)(F)C(C)(C)O)c4)c3cc21] | 7.92 |
| 181 | 3-methoxy-1,3,5-trimethyl-8- [[(1R)-1-[3-(1,1-difluoro-2- hydroxy-2-methyl-propyl)-5- fluoro-2-methyl- phenyl]ethyl]amino]pyrrolo[2,3- g]phthalazin-2-one | 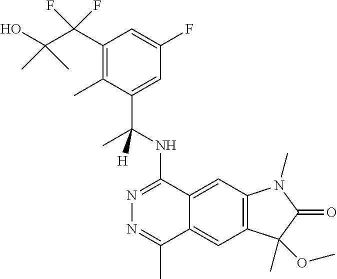 [SMILES: COC1(C)C(=O)N(C)c2cc3c(N[C@H](C)c4cc(F)cc(C(F)(F)C(C)(C)O)c4C)nnc(C)c3cc21] | 7.88 |
| 182 | 3-methoxy-1,3,5-trimethyl-8- [[(1R)-1-[3-(1,1-difluoro-2- hydroxy-ethyl)-2,5-dimethyl- phenyl]ethyl]amino]pyrrolo[2,3- g]phthalazin-2-one | 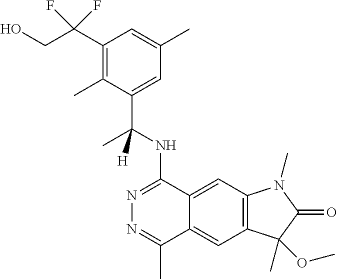 [SMILES: COC1(C)C(=O)N(C)c2cc3c(N[C@H](C)c4cc(C)cc(C(F)(F)CO)c4C)nnc(C)c3cc21] | 8.01 |
| 183 | 3-methoxy-1,3,5-trimethyl-8- [[(1R)-1-[3-(1,1-difluoro-2- hydroxy-ethyl)-5-fluoro-2-methyl- phenyl]ethyl]amino]pyrrolo[2,3- g]phthalazin-2-one | 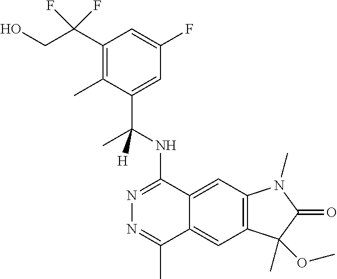 [SMILES: COC1(C)C(=O)N(C)c2cc3c(N[C@H](C)c4cc(F)cc(C(F)(F)CO)c4C)nnc(C)c3cc21] | 7.96 |
| 184 | 3-ethoxy-1,3,8-trimethyl-5-[[(1R)- 1-[3-(1,1-difluoro-2-hydroxy-2- methyl- propyl)phenyl]ethyl]amino]pyrrolo [3,2-g]phthalazin-2-one | 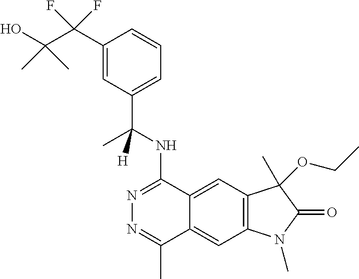 [SMILES: CCOC1(C)C(=O)N(C)c2cc3c(C)nnc(N[C@H](C)c4cccc(C(F)(F)C(C)(C)O)c4)c3cc21] | 7.83 |
| 185 | 3-ethoxy-1,3,8-trimethyl-5-[[(1R)- 1-[3-(1,1-difluoro-2-hydroxy-2- methyl-propyl)-2-methyl- phenyl]ethyl]amino]pyrrolo[3,2- g]phthalazin-2-one | 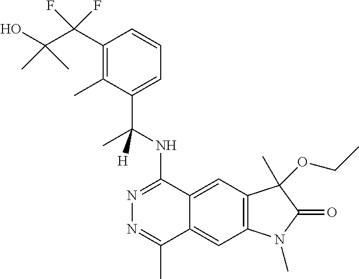 [SMILES: CCOC1(C)C(=O)N(C)c2cc3c(C)nnc(N[C@H](C)c4cccc(C(F)(F)C(C)(C)O)c4C)c3cc21] | 7.61 |
| 186 | 1′,5′-dimethyl-8′-[[(1R)-1-[3-(1,1- difluoro-2-hydroxy-2-methyl- propyl)-2-methyl- phenyl]ethyl]amino]spiro [cyclopentane-1,3′-pyrrolo[2,3- g]phthalazine]-2′-one | 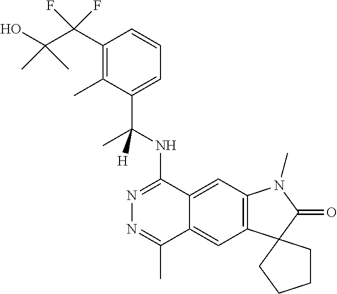 [SMILES: Cc1c([C@@H](C)Nc2nnc(C)c3cc4c(cc23)N(C)C(=O)C42CCCC2)cccc1C(F)(F)C(C)(C)O] | 8.08 |
| 187 | 1′,5′-dimethyl-8′-[[(1R)-1-[3-(1,1- difluoro-2-hydroxy-ethyl)-5- fluoro- phenyl]ethyl]amino]spiro [cyclopentane-1,3′-pyrrolo[2,3- g]phthalazine]-2′-one | 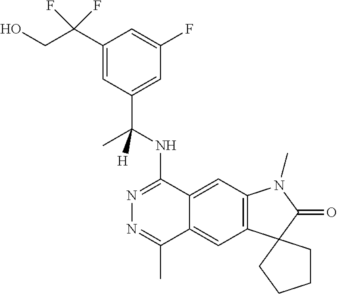 [SMILES: Cc1nnc(N[C@H](C)c2cc(F)cc(C(F)(F)CO)c2)c2cc3c(cc12)C1(CCCC1)C(=O)N3C] | 7.66 |
| 188 | 1′,5′-dimethyl-8′-[[(1R)-1-[3-(1,1- difluoro-2-hydroxy-2-methyl- propyl)phenyl]ethyl]amino]spiro [cyclopentane-1,3′-pyrrolo[2,3- g]phthalazine]-2′-one | 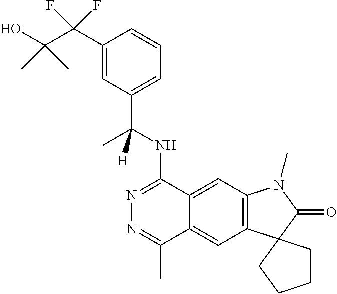 [SMILES: Cc1nnc(N[C@H](C)c2cccc(C(F)(F)C(C)(C)O)c2)c2cc3c(cc12)C1(CCCC1)C(=O)N3C] | 8.03 |
| 189 | 1′,5′-dimethyl-8′-[[(1R)-1-[3-(1,1- difluoro-2-hydroxy-ethyl)-2- fluoro- phenyl]ethyl]amino]spiro[cyclope ntane-1,3′-pyrrolo[2,3-g] phthalazine]-2′-one | 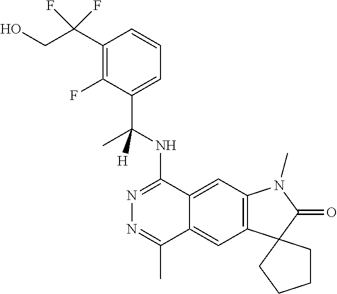 [SMILES: Cc1nnc(N[C@H](C)c2cccc(C(F)(F)CO)c2F)c2cc3c(cc12)C1(CCCC1)C(=O)N3C] | 8.17 |
| 190 | 3-ethoxy-1,3,8-trimethyl-5-[[(1R)- 1-[3-(1,1-difluoro-2-hydroxy-2- methyl-propyl)-2-fluoro- phenyl]ethyl]amino]pyrrolo[3,2- g]phthalazin-2-one | 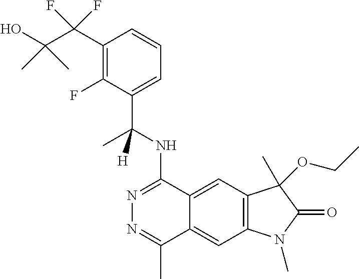 [SMILES: CCOC1(C)C(=O)N(C)c2cc3c(C)nnc(N[C@H](C)c4cccc(C(F)(F)C(C)(C)O)c4F)c3cc21] | 7.78 |
| 191 | 5-[[(1R)-1-[3-(1,1-difluoro-2- hydroxy-2-methyl-propyl)-2- methyl-phenyl]ethyl]amino]-3- ethyl-3-methoxy-1,8-dimethyl- pyrrolo[2,3-g]phthalazin-2-one | 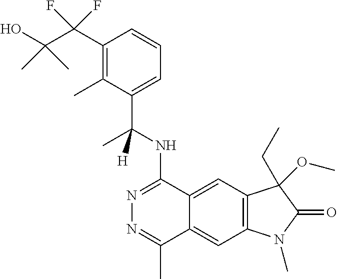 [SMILES: CCC1(OC)C(=O)N(C)c2cc3c(C)nnc(N[C@H](C)c4cccc(C(F)(F)C(C)(C)O)c4C)c3cc21] | 7.63 |
| 192 | 5-[[(1R)-1-[3-(1,1-difluoro-2- hydroxy-2-methyl- propyl)phenyl]ethyl]amino]-3- ethyl-3-methoxy-1,8-dimethyl- pyrrolo[2,3-g]phthalazin-2-one | 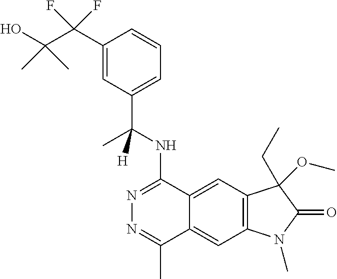 [SMILES: CCC1(OC)C(=O)N(C)c2cc3c(C)nnc(N[C@H](C)c4cccc(C(F)(F)C(C)(C)O)c4)c3cc21] | 7.79 |
| 193 | 1′,5′-dimethyl-8′-[[(1R)-1-[3-(1,1- difluoro-2-hydroxy-ethyl)-2- methyl-phenyl] ethyl]amino]spiro[cyclopentane- 1,3′-pyrrolo[2,3- g]phthalazine]-2′-one | 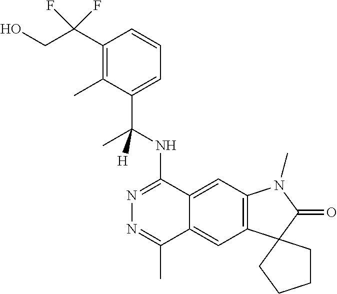 [SMILES: Cc1c([C@@H](C)Nc2nnc(C)c3cc4c(cc23)N(C)C(=O)C42CCCC2)cccc1C(F)(F)CO] | 8.06 |
| 194 | 1′,5′-dimethyl-8′-[[(1R)-1-[3-(1,1- difluoro-2-hydroxy-2-methyl- propyl)-2-fluoro- phenyl]ethyl]amino]spiro [cyclopentane-1,3′-pyrrolo[2,3- g]phthalazine]-2′-one | 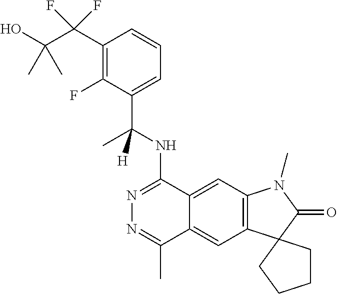 [SMILES: Cc1nnc(N[C@H](C)c2cccc(C(F)(F)C(C)(C)O)c2F)c2cc3c(cc12)C1(CCCC1)C(=O)N3C] | 8.07 |
| 195 | 3-ethoxy-1,3,8-trimethyl-5-[[(1R)- 1-[3-(1,1-difluoro-2-hydroxy- ethyl)phenyl]ethyl]amino]pyrrolo [3,2-g]phthalazin-2-one | 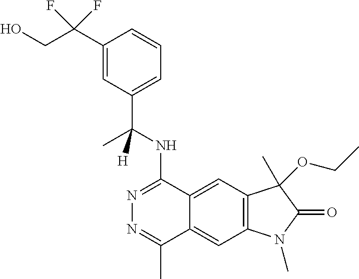 [SMILES: CCOC1(C)C(=O)N(C)c2cc3c(C)nnc(N[C@H](C)c4cccc(C(F)(F)CO)c4)c3cc21] | 7.68 |
| 196 | 5-[[(1R)-1-[3-(1,1-difluoro-2- hydroxy- ethyl)phenyl]ethyl]amino]-3-ethyl- 3-methoxy-1,8-dimethyl- pyrrolo[2,3-g]phthalazin-2-one | 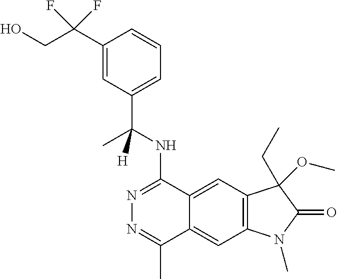 [SMILES: CCC1(OC)C(=O)N(C)c2cc3c(C)nnc(N[C@H](C)c4cccc(C(F)(F)CO)c4)c3cc21] | 7.60 |
| 197 | 5-[[(1R)-1-[3-(1,1-difluoro-2- hydroxy-ethyl)-2-fluoro- phenyl]ethyl]amino]-3-ethyl-3- methoxy-1,8-dimethyl- pyrrolo[2,3-g]phthalazin-2-one | 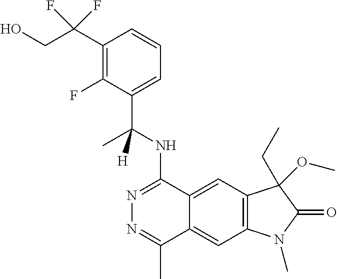 [SMILES: CCC1(OC)C(=O)N(C)c2cc3c(C)nnc(N[C@H](C)c4cccc(C(F)(F)CO)c4F)c3cc21] | 7.84 |
| 198 | 5-[[(1R)-1-[3-(1,1-difluoro-2- hydroxy-ethyl)-2-methyl- phenyl]ethyl]amino]-3-ethyl-3- methoxy-1,8-dimethyl- pyrrolo[2,3-g]phthalazin-2-one | 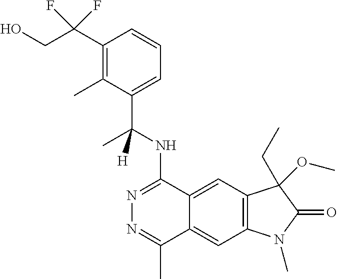 [SMILES: CCC1(OC)C(=O)N(C)c2cc3c(C)nnc(N[C@H](C)c4cccc(C(F)(F)CO)c4C)c3cc21] | 7.71 |
| 199 | 3-ethoxy-1,3,8-trimethyl-5-[[(1R)- 1-[3-(1,1-difluoro-2-hydroxy- ethyl)-2-methyl- phenyl]ethyl]amino]pyrrolo[3,2- g]phthalazin-2-one | 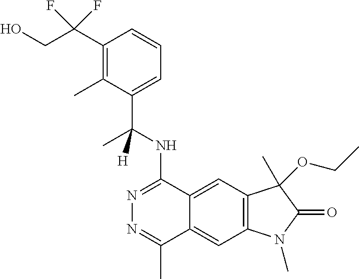 [SMILES: CCOC1(C)C(=O)N(C)c2cc3c(C)nnc(N[C@H](C)c4cccc(C(F)(F)CO)c4C)c3cc21] | 7.66 |
| 200 | 5-ethyl-3-methoxy-1,3-dimethyl- 8-[[(1R)-1-[3-(1,1-difluoro-2- hydroxy-2-methyl-propyl)-2- fluoro- phenyl]ethyl]amino]pyrrolo[2,3- g]phthalazin-2-one | 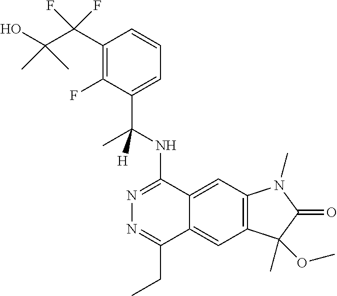 [SMILES: CCc1nnc(N[C@H](C)c2cccc(C(F)(F)C(C)(C)O)c2F)c2cc3c(cc12)C(C)(OC)C(=O)N3C] | 7.88 |
| 201 | 3-ethoxy-1,3,8-trimethyl-5-[[(1R)- 1-[3-(1, 1-difluoro-2-hydroxy- ethyl)-2-fluoro- phenyl]ethyl]amino]pyrrolo[3,2- g]phthalazin-2-one | 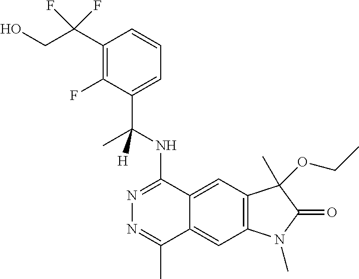 [SMILES: CCOC1(C)C(=O)N(C)c2cc3c(C)nnc(N[C@H](C)c4cccc(C(F)(F)CO)c4F)c3cc21] | 7.74 |
| 202 | 3-ethyl-3-methoxy-1,5-dimethyl- 8-[[(1R)-1-[3-(1,1-difluoro-2- hydroxy-2-methyl- propyl)phenyl]ethyl]amino]pyrrolo [2,3-g]phthalazin-2-one | 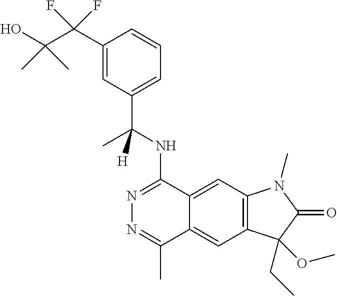 [SMILES: CCC1(OC)C(=O)N(C)c2cc3c(N[C@H](C)c4cccc(C(F)(F)C(C)(C)O)c4)nnc(C)c3cc21] | 7.97 |
| 203 | 3-ethyl-3-methoxy-1,5-dimethyl- 8-[[(1R)-1-[3-(1,1-difluoro-2- hydroxy-ethyl)-2-methyl- phenyl]ethyl]amino]pyrrolo[2,3- g]phthalazin-2-one | 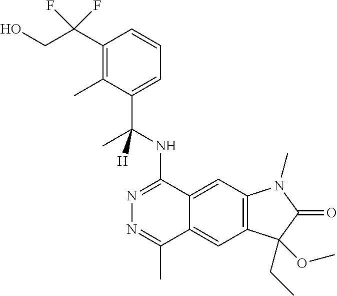 [SMILES: CCC1(OC)C(=O)N(C)c2cc3c(N[C@H](C)c4cccc(C(F)(F)CO)c4C)nnc(C)c3cc21] | 7.93 |
| 204 | 3-ethyl-3-methoxy-1,5-dimethyl- 8-[[(1R)-1-[3-(1,1-difluoro-2- hydroxy-2-methyl-propyl)-2- fluoro- phenyl]ethyl]amino]pyrrolo[2,3- g]phthalazin-2-one | 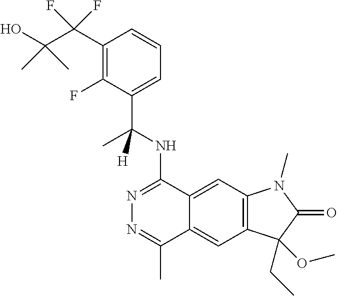 [SMILES: CCC1(OC)C(=O)N(C)c2cc3c(N[C@H](C)c4cccc(C(F)(F)C(C)(C)O)c4F)nnc(C)c3cc21] | 8.00 |
| 205 | 3-tert-butyl-5-[[(1R)-1-[3- (difluoromethyl)phenyl]ethyl]amino]- 1,8-dimethyl-imidazo[4,5- g]phthalazin-2-one | 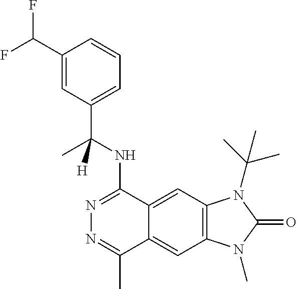 [SMILES: Cc1nnc(N[C@H](C)c2cccc(C(F)F)c2)c2cc3c(cc12)n(C)c(=O)n3C(C)(C)C] | 7.93 |
| 206 | 3-ethyl-3-methoxy-1,5-dimethyl- 8-[[(1R)-1-[3-(1,1-difluoro-2- hydroxy- ethyl)phenyl]ethyl]amino]pyrrolo [2,3-g]phthalazin-2-one | 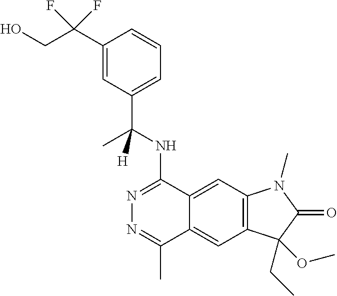 [SMILES: CCC1(OC)C(=O)N(C)c2cc3c(N[C@H](C)c4cccc(C(F)(F)CO)c4)nnc(C)c3cc21] | 7.84 |
| 207 | 3-ethyl-3-methoxy-1,5-dimethyl- 8-[[(1R)-1-[3-(1,1-difluoro-2- hydroxy-ethyl)-2-fluoro- phenyl]ethyl]amino]pyrrolo[2,3- g]phthalazin-2-one | 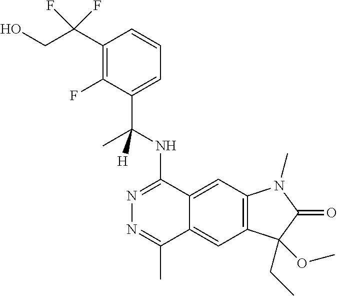 [SMILES: CCC1(OC)C(=O)N(C)c2cc3c(N[C@H](C)c4cccc(C(F)(F)CO)c4F)nnc(C)c3cc21] | 8.07 |
| 208 | 5-[[(1R)-1-[3-(1,1-difluoro-2- hydroxy-2-methyl-propyl)-2- methyl-phenyl]ethyl]amino]-1,3,8- trimethyl-imidazo[4,5- g]phthalazin-2-one | 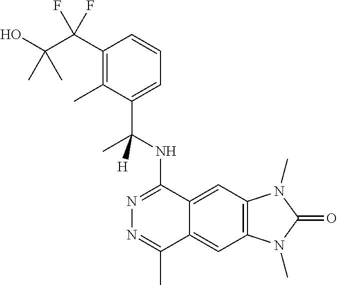 [SMILES: Cc1c([C@@H](C)Nc2nnc(C)c3cc4c(cc23)n(C)c(=O)n4C)cccc1C(F)(F)C(C)(C)O] | |
| 209 | 3-ethyl-3-methoxy-1,5-dimethyl- 8-[[(1R)-1-[3-(1,1-difluoro-2- hydroxy-2-methyl-propyl)-2- methyl- phenyl]ethyl]amino]pyrrolo[2,3- g]phthalazin-2-one | 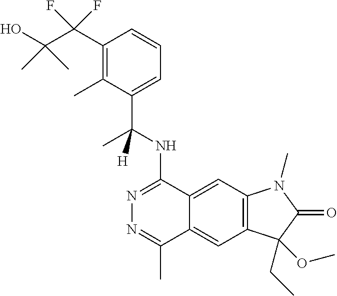 [SMILES: CCC1(OC)C(=O)N(C)c2cc3c(N[C@H](C)c4cccc(C(F)(F)C(C)(C)O)c4C)nnc(C)c3cc21] | 8.02 |
| 210 | 3-cyclopropyl-5-[[(1R)-1-[3-(1,1- difluoro-2-hydroxy-2-methyl- propyl)-2-methyl- phenyl]ethyl]amino]-1,8-dimethyl- imidazo[4,5-g]phthalazin-2-one | 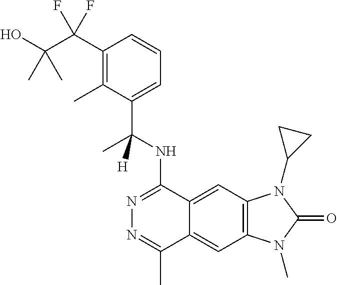 [SMILES: Cc1c([C@@H](C)Nc2nnc(C)c3cc4c(cc23)n(C2CC2)c(=O)n4C)cccc1C(F)(F)C(C)(C)O] | 7.88 |
| 211 | 5-[[(1R)-1-[3-(difluoromethyl)-2- methyl-phenyl]ethyl]amino]-1,3,8- trimethyl-imidazo[4,5- g]phthalazin-2-one | 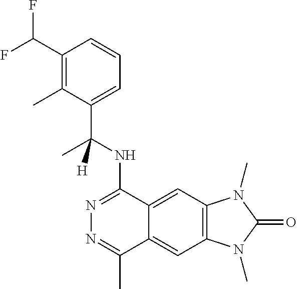 [SMILES: Cc1c(C(F)F)cccc1[C@@H](C)Nc1nnc(C)c2cc3c(cc12)n(C)c(=O)n3C] | 8.01 |
| 212 | 5-[[(1R)-1-[3-(1,1-difluoro-2- hydroxy-ethyl)-2,5-dimethyl- phenyl]ethyl]amino]-1,3,8- trimethyl-imidazo[4,5- g]phthalazin-2-one | 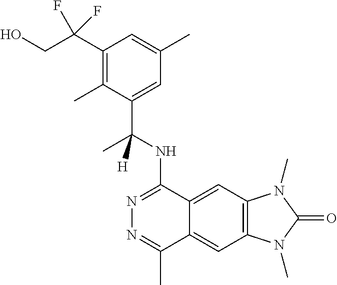 [SMILES: Cc1cc([C@@H](C)Nc2nnc(C)c3cc4c(cc23)n(C)c(=O)n4C)c(C)c(C(F)(F)CO)c1] | 7.90 |
| 213 | 3-cyclopropyl-5-[[(1R)-1-[3- (difluoromethyl)-2-methyl- phenyl]ethyl]amino]-1,8-dimethyl- imidazo[4,5-g]phthalazin-2-one | 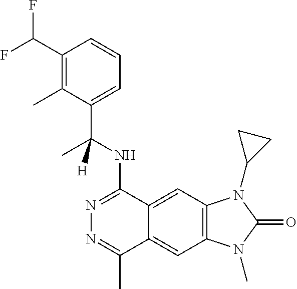 [SMILES: Cc1c(C(F)F)cccc1[C@@H](C)Nc1nnc(C)c2cc3c(cc12)n(C1CC1)c(=O)n3C] | 7.96 |
| 214 | 5-[[(1R)-1-[3-(1,1-difluoro-2- hydroxy-ethyl)-5-fluoro-2-methyl- phenyl]ethyl]amino]-1,3,8- trimethyl-imidazo[4,5- g]phthalazin-2-one | 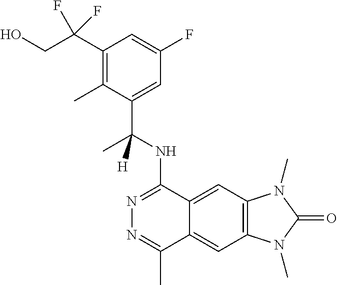 [SMILES: Cc1c([C@@H](C)Nc2nnc(C)c3cc4c(cc23)n(C)c(=O)n4C)cc(F)cc1C(F)(F)CO] | |
| 215 | 5-[[(1R)-1-[3-(1, 1-difluoro-2- hydroxy-2-methyl-propyl)-5- fluoro-2-methyl- phenyl]ethyl]amino]-1,3,8- trimethyl-imidazo[4,5- g]phthalazin-2-one | 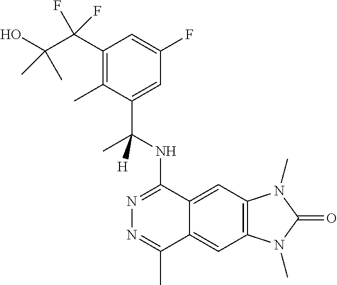 [SMILES: Cc1c([C@@H](C)Nc2nnc(C)c3cc4c(cc23)n(C)c(=O)n4C)cc(F)cc1C(F)(F)C(C)(C)O] | |
| 216 | 8-[[(1R)-1-[3-(1,1-difluoro-2- hydroxy-2-methyl-propyl)-2,4- difluoro-phenyl]ethyl]amino]-3- methoxy-1,3,5-trimethyl- pyrrolo[3,2-g]phthalazin-2-one | 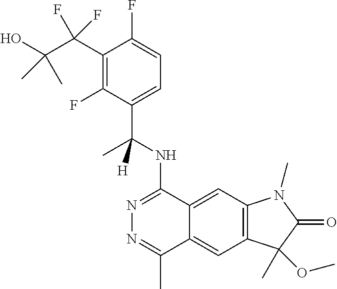 [SMILES: COC1(C)C(=O)N(C)c2cc3c(N[C@H](C)c4ccc(F)c(C(F)(F)C(C)(C)O)c4F)nnc(C)c3cc21] | |

What is claimed is:

1. A compound of Formula (I): 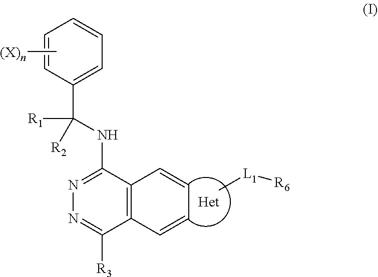
or a pharmaceutically acceptable salt thereof,
wherein
each X is independently halogen, alkyl, alkoxy, amino, amido, nitrile, acyl, cycloalkyl, heterocyclyl, or heteroaryl, and n is an integer from 1-5, and/or two X groups together with the atoms to which they are attached form a heterocyclyl or heteroaryl ring;
R₁ and R₂ are each independently hydrogen, alkyl, or R₁ and R₂ together with the atom to which they are attached form a cycloalkyl or heterocyclyl, wherein at least one of R₁ and R₂ is not hydrogen;
R₃ is hydrogen, alkyl, —(C═O)—OR_{A}, —(C═O)—N(R_{A})₂, cycloalkyl, heterocyclyl, aryl or heteroaryl, wherein each R_{A} is independently H or alkyl; and 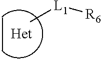
is a nitrogen-containing heterocyclyl substituted with L₁-R₆ and 0-6 substituents independently selected from R₈, R₉, R₁₀, and R, wherein
L₁ is absent, alkylene, alkenylene, or alkynylene;
R₆ is alkyl, —O-alkyl, cycloalkyl, or heterocyclyl;
R₈ and R₉ are each independently H, halogen, or alkyl, or an R₈ and R₉ together with the carbon atom to which they are attached form a C₃₋₆cycloalkyl, a 3- to 6-membered heterocyclyl, or a carbonyl;
R₁₀ is H, halogen, or -L₂-R₇, wherein L₂ is absent, alkylene, alkenylene, or alkynylene; and R₇ is H, alkyl, —O-alkyl, cycloalkyl, or heterocyclyl; and
R₁₁ is H, halogen, or alkyl, or an R₁₀ and R₁₁ together with the carbon atom to which they are attached form a C₃₋₆cycloalkyl, a 3- to 6-membered heterocyclyl, or a carbonyl.

2. The compound of claim 1, wherein 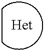
is a 5- or 6-membered heterocyclyl comprising 1-3 heteroatoms selected from nitrogen and oxygen, wherein at least one of the heteroatoms is nitrogen.

3. The compound of claim 1 or 2, wherein 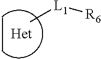
is: 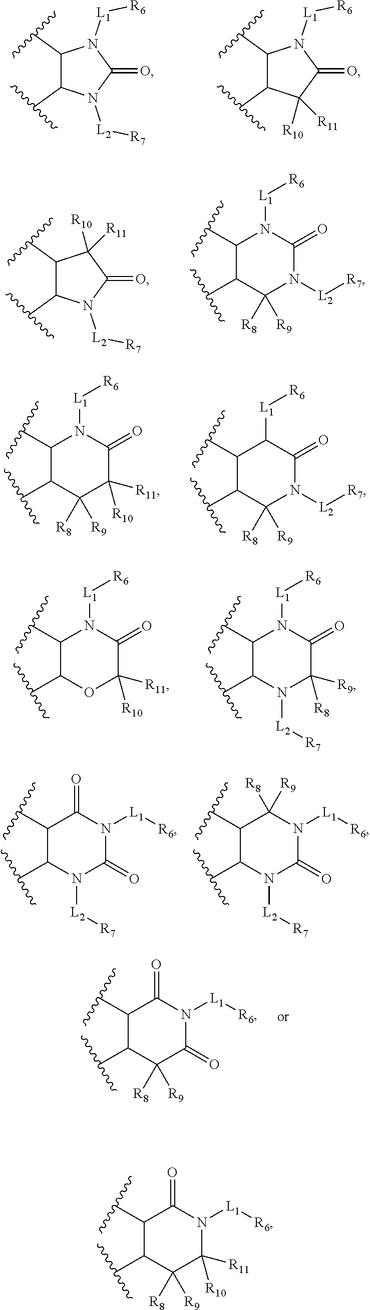
wherein
R₈ and R₉ are each independently H, F, or C₁₋₅alkyl, or an R₈ and R₉ together with the carbon atom to which they are attached form a C₃₋₆cycloalkyl or a 3- to 6-membered heterocyclyl;
R₁₀ is H, F, C₁₋₅alkyl, or -L₂-R₇; and
R₁₁ is H, F, or C₁₋₅alkyl, or an R₁₀ and R₁₁ together with the carbon atom to which they are attached form a C₃₋₆cycloalkyl or a 3- to 6-membered heterocyclyl.

4. The compound of any one of claims 1-3, wherein L₁ and L₂ is each independently absent or C₁₋₅alkylene.

5. The compound of any one of claims 1-4, wherein L₁ is C₁₋₅alkylene.

6. The compound of any one of claims 1-5, wherein L₁ is —CH₂— or —CH₂CH₂—.

7. The compound of any one of claims 1-5, wherein L₂ is absent.

8. The compound of any one of claims 1-3, wherein L₁ and L₂ are absent.

9. The compound of any one of claims 1-8, wherein R₆ is C₁₋₅alkyl, —O—C₁₋₅alkyl, C₃₋₆cycloalkyl, or 4- to 6-membered heterocyclyl.

10. The compound of any one of claims 1-9, wherein R₆ is 3- to 6-membered heterocyclyl.

11. The compound of claim 9 or 10, wherein the 3- to 6-membered heterocyclyl is

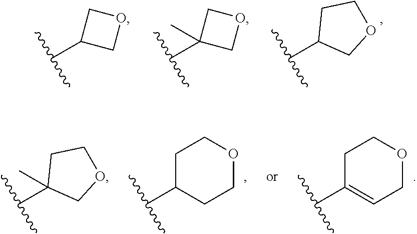

12. The compound of any one of claims 1-8, wherein R₆ is C₃₋₆cycloalkyl.

13. The compound of claim 9 or 12, wherein the C₃₋₆cycloalkyl is 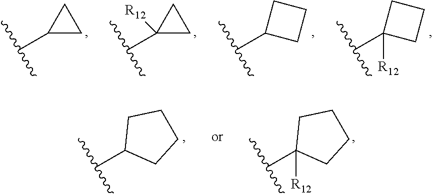
wherein R₁₂ is C₁₋₅alkyl.

14. The compound of claim 13, wherein R₁₂ is —CH₃, —CF₃, or —CF₂H.

15. The compound of any one of claims 1-9, wherein R₆ is C₁₋₅alkyl.

16. The compound of claim 9 or 15, wherein the C₁₋₅alkyl is methyl, ethyl, isopropyl, tert-butyl, —CH₂CN, —CH(CH₃)CN, —CH₂CH₂OCH₃, —CH₂CF₃, CH₂CF₂H, —CH(CH₃)CF₃, CH(CH₃)CF₂H, —C(CH₃)₂CF₃, or —C(CH₃)₂CF₂H.

17. The compound of any one of claims 1-9, wherein R₆ is methyl, ethyl, isopropyl, tert-butyl, —CH₂CN, —CH(CH₃)CN, —CH₂CF₃, —CH₂CH₂OCH₃, cyclopropyl, cyclobutyl, cyclopentyl, —CH₂cyclopropyl, —CH₂cyclobutyl, —CH₂cyclopentyl, 3-oxetanyl, or 3-tetrahydrofuranyl.

18. The compound of claim 9 or 17, wherein R₆ is methyl.

19. The compound of any one of claims 1-18, wherein R₈ is H, F, or C₁₋₅alkyl.

20. The compound of any one of claims 1-19, wherein R₉ is H, F, or C₁₋₅alkyl.

21. The compound of claim 19 or 20, wherein the C₁₋₅alkyl is methyl or ethyl.

22. The compound of any one of claims 1-18, wherein an R₈ and R₉ together with the carbon atom to which they are attached form a C₃₋₆cycloalkyl.

23. The compound of any one of claims 1-22, wherein R₁₀ is H, F, C₁₋₅alkyl, or -L₂-R₇.

24. The compound of any one of claims 1-23, wherein R₇ is C₁₋₅alkyl, C₃₋₅cycloalkyl, or 4- to 6-membered heterocyclyl.

25. The compound of any one of claims 1-25, wherein R₇ is C₁₋₅alkyl.

26. The compound of any one of claims 1-26, wherein R₇ is methyl.

27. The compound of any one of claims 1-23, wherein R₁₀ is methyl.

28. The compound of any one of claims 1-27, wherein R₁₁ is H, F, or C₁₋₅alkyl.

29. The compound of any one of claims 1-28, wherein R₁₁ is methyl or —O-methyl.

30. The compound of any one of claims 1-22, wherein R₁₀ and R₁₁ together with the carbon atom to which they are attached form C₃₋₆cycloalkyl or a 3- to 6-membered heterocyclyl.

31. The compound of claim 29, wherein R₁₀ and R₁₁ together with the carbon atom to which they are attached form

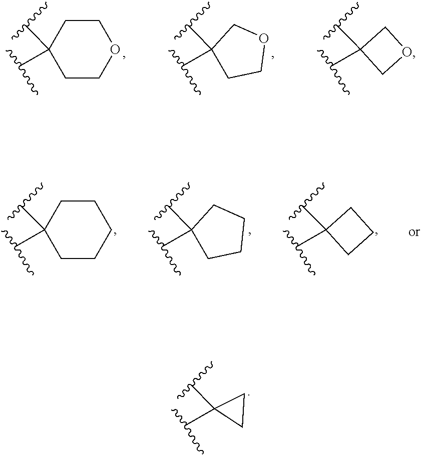

32. The compound of any one of claims 1-31, wherein X is an alkyl substituted with one or more halogen, hydroxyl, alkoxy, amino, or combination thereof.

33. The compound of any one of claims 1-31, wherein X is an alkyl substituted with one or more halogen, —OH, —O—(C₁₋₅ alkyl), —NH₂, —NH—(C₁₋₅ alkyl), C₁₋₂ haloalkyl, and/or —O—(C₁₋₂ haloalkyl).

34. The compound of any one of claims 1-31, wherein each X is independently halogen, haloalkyl or amino.

35. The compound of claim 34, wherein the haloalkyl is a fluoroalkyl.

36. The compound of any one of claims 1-35, wherein each X is independently —CH₂F, —CHF₂, —CF₃, —CF₂CH₂OH, —CF₂C(CH₃)₂OH, —CF₂C(CH₃)(CH₂OMe)OH, —CF₂C(CH₃)(CH₂NHMe)OH, —CF₂C(CH₃)(CH₂NMe₂)OH, —CF₂C(CH₃)₂NH₂, F, or —NH₂.

37. The compound of any one of claims 1-35, wherein two X groups together with the atoms to which they are attached form a 5- or 6-membered heterocyclyl ring.

38. The compound of any one of claims 1-35, wherein two X groups together with the atoms to which they are attached form a 5-membered heterocyclyl ring.

39. The compound of any one of claims 1-38, wherein n is 1.

40. The compound of any one of claims 1-38, wherein n is 2.

41. The compound of any one of claims 1-31, wherein 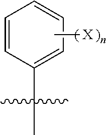
is: 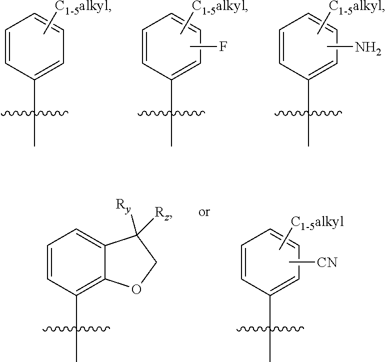
wherein R_{y} and R_{z} are each independently H, F, or alkyl, or R_{y} and R_{z} together with the carbon atom to which they are attached form a C₃₋₆cycloalkyl.

42. The compound of claim 41, wherein the C₁₋₅alkyl is —CH₂F, —CHF₂, —CF₃, —CH₂CF₃, —CF₂CH₂OH, or —CF₂C(Me)₂OH.

43. The compound of any one of claims 1-31, wherein 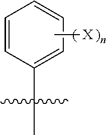
is:

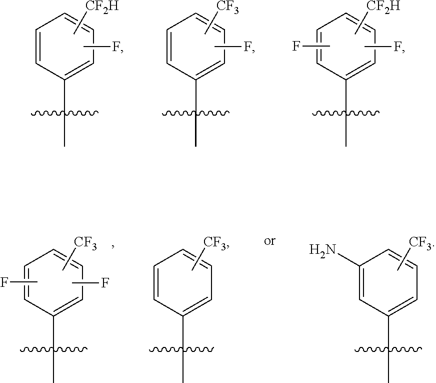

44. The compound of any one of claims 1-31, wherein 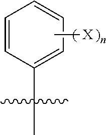
is:

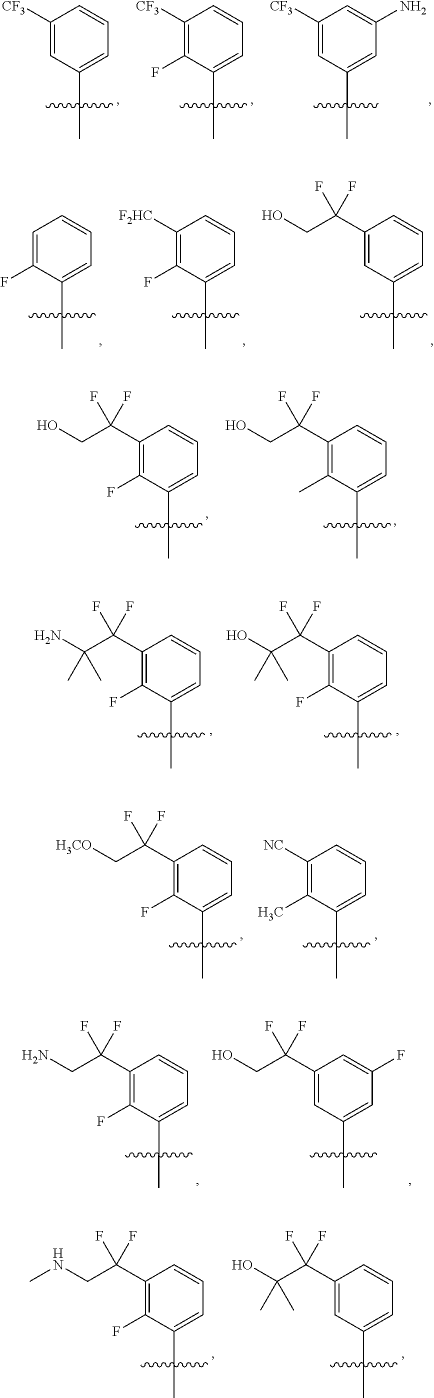

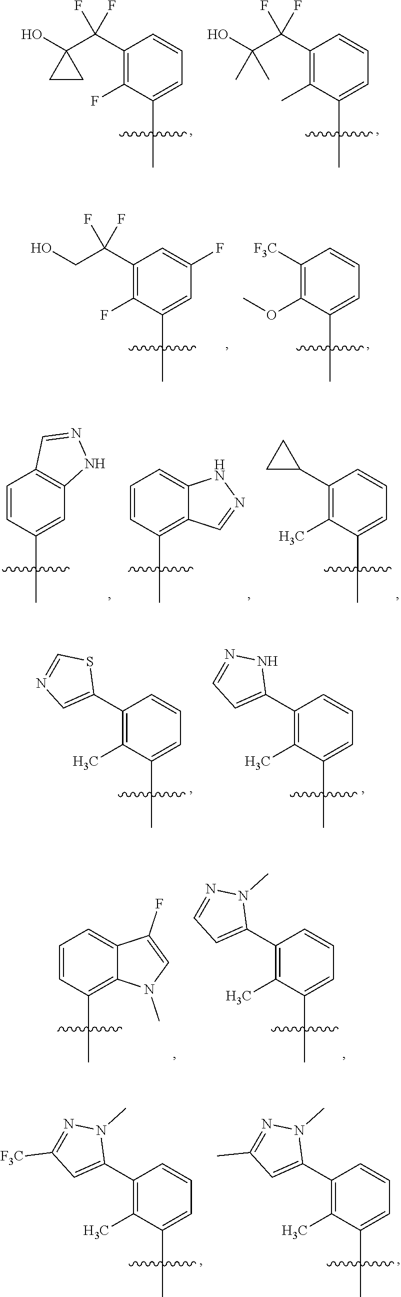

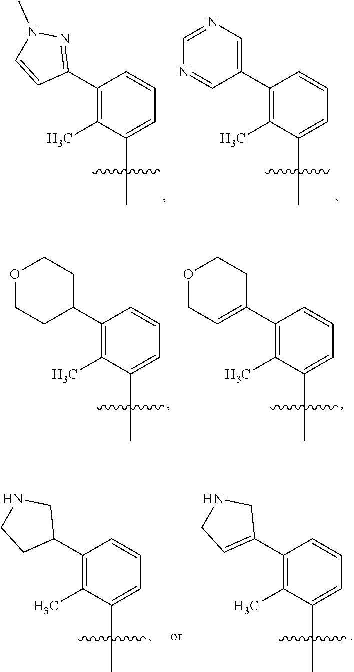

45. The compound of any one of claims 1-44, wherein R₃ is methyl, ethyl, isopropyl, n-propyl, —CH₂OH, —CH₂OCH₃, —CH₂N(CH₃)₂, —CH(OH)(CH₃)₂ or —CH₂(OH)CH₃.

46. The compound of any one of claims 1-44, wherein R₃ is methyl.

47. The compound of any one of claims 1-46, wherein R₁ is methyl and R₂ is H.

48. The compound of any one of claims 1-46, wherein R₁ and R₂ together with the atom to which they are attached form a cyclopropyl.

49. The compound of any one of claims 1-22 and 32-48, having the structure of Formula (Ia): 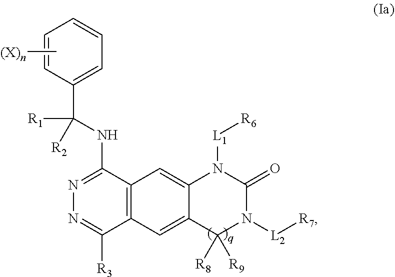
wherein q is 0 or 1.

50. The compound of any one of claims 1-48, having the structure of Formula (Ib): 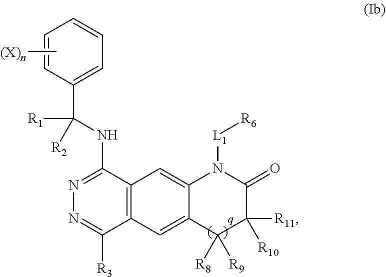
wherein q is 0 or 1.

51. The compound of claim 49 or 50, wherein q is 0.

52. The compound of any one of claims 1-18 and 23-48, having the structure of Formula (Ic-1):

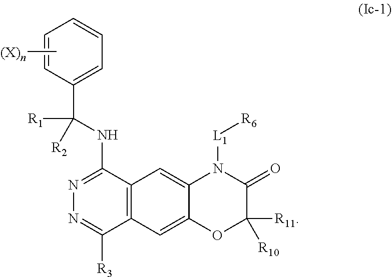

53. The compound of claim 1, wherein the compound is: 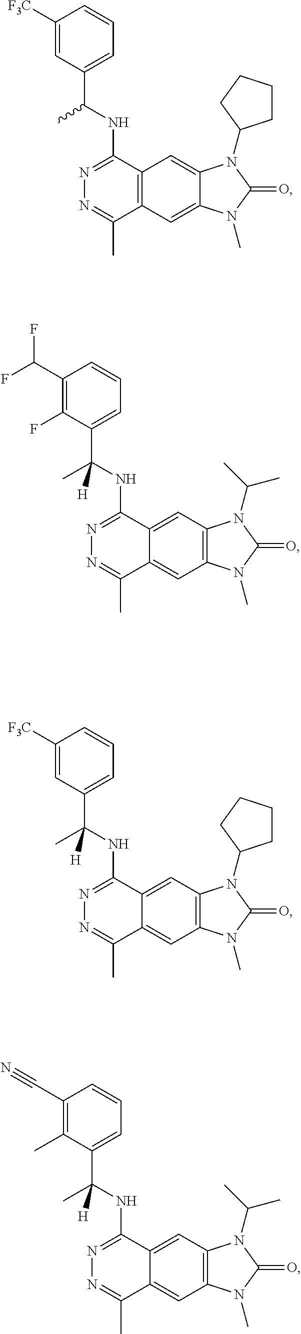 [SMILES: Cc1nnc(NC(C)c2cccc(C(F)(F)F)c2)c2cc3c(cc12)n(C)c(=O)n3C1CCCC1 ; Cc1nnc(N[C@H](C)c2cccc(C(F)F)c2F)c2cc3c(cc12)n(C)c(=O)n3C(C)C ; Cc1nnc(N[C@H](C)c2cccc(C(F)(F)F)c2)c2cc3c(cc12)n(C)c(=O)n3C1CCCC1 ; Cc1c(C#N)cccc1[C@@H](C)Nc1nnc(C)c2cc3c(cc12)n(C(C)C)c(=O)n3C] 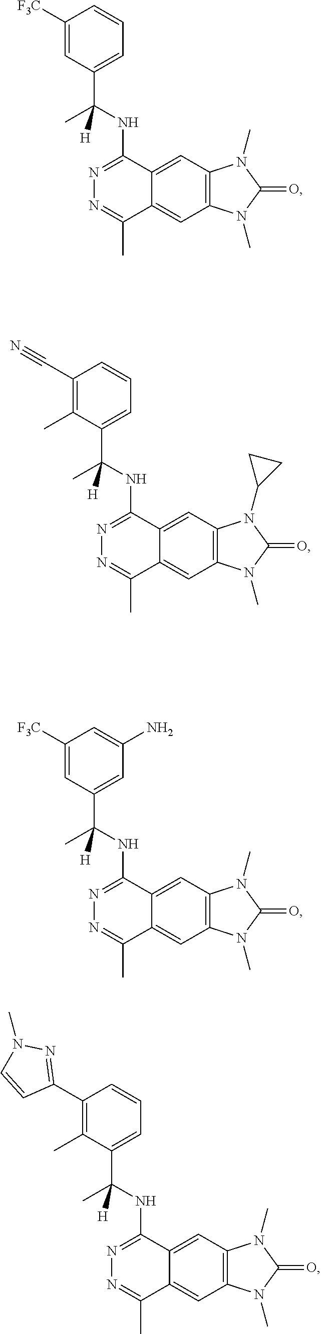 [SMILES: Cc1nnc(N[C@H](C)c2cccc(C(F)(F)F)c2)c2cc3c(cc12)n(C)c(=O)n3C ; Cc1c(C#N)cccc1[C@@H](C)Nc1nnc(C)c2cc3c(cc12)n(C1CC1)c(=O)n3C ; Cc1nnc(N[C@H](C)c2cc(N)cc(C(F)(F)F)c2)c2cc3c(cc12)n(C)c(=O)n3C ; Cc1c(-c2ccn(C)n2)cccc1[C@@H](C)Nc1nnc(C)c2cc3c(cc12)n(C)c(=O)n3C] 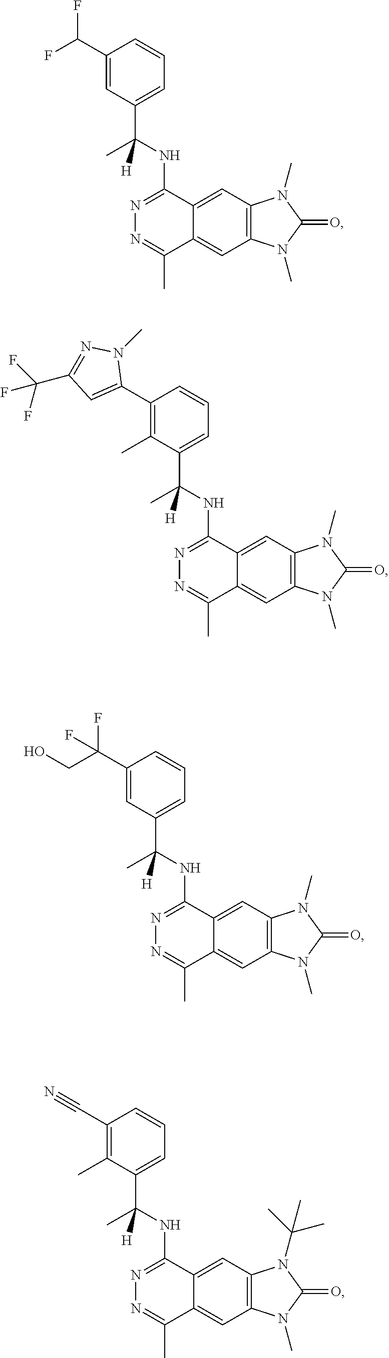 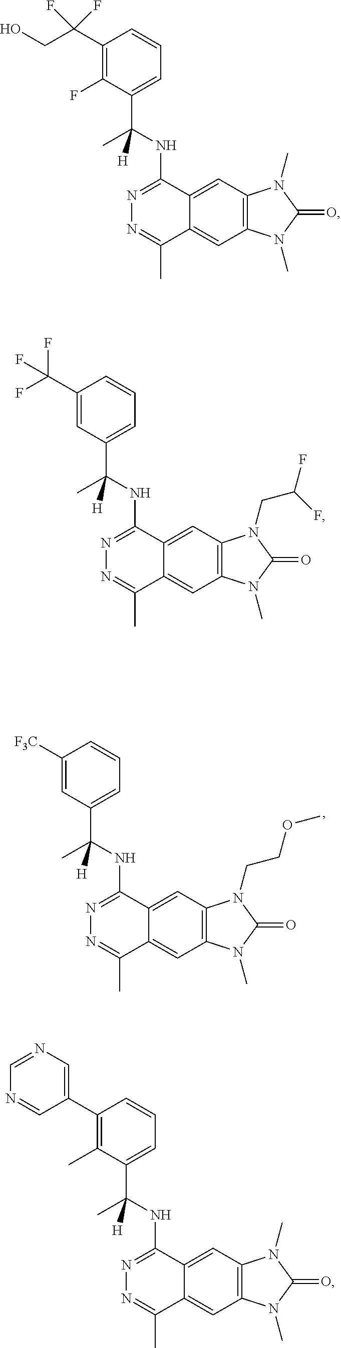 [SMILES: Cc1nnc(N[C@H](C)c2cccc(C(F)(F)CO)c2F)c2cc3c(cc12)n(C)c(=O)n3C ; Cc1nnc(N[C@H](C)c2cccc(C(F)(F)F)c2)c2cc3c(cc12)n(C)c(=O)n3CC(F)F ; COCCn1c(=O)n(C)c2cc3c(C)nnc(N[C@H](C)c4cccc(C(F)(F)F)c4)c3cc21 ; Cc1c(-c2cncnc2)cccc1[C@@H](C)Nc1nnc(C)c2cc3c(cc12)n(C)c(=O)n3C] 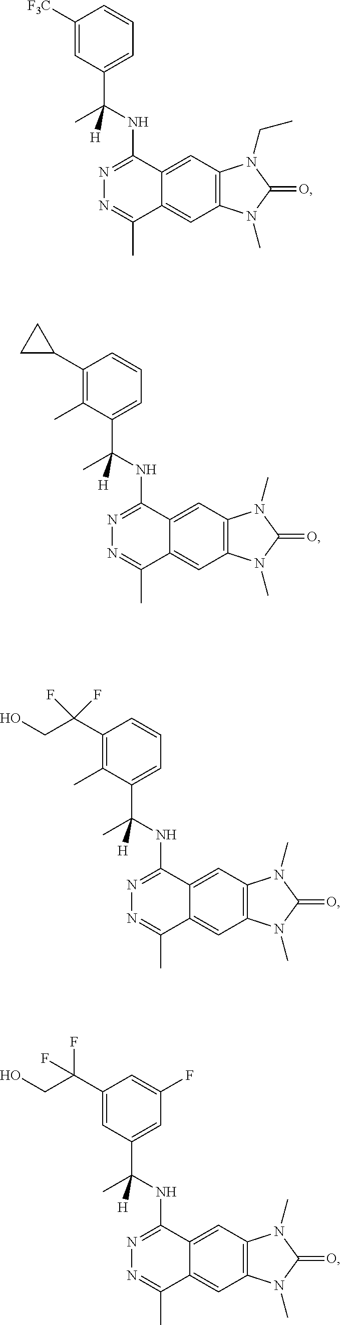 [SMILES: CCn1c(=O)n(C)c2cc3c(C)nnc(N[C@H](C)c4cccc(C(F)(F)F)c4)c3cc21 ; Cc1c(C2CC2)cccc1[C@@H](C)Nc1nnc(C)c2cc3c(cc12)n(C)c(=O)n3C ; Cc1c([C@@H](C)Nc2nnc(C)c3cc4c(cc23)n(C)c(=O)n4C)cccc1C(F)(F)CO ; Cc1nnc(N[C@H](C)c2cc(F)cc(C(F)(F)CO)c2)c2cc3c(cc12)n(C)c(=O)n3C] 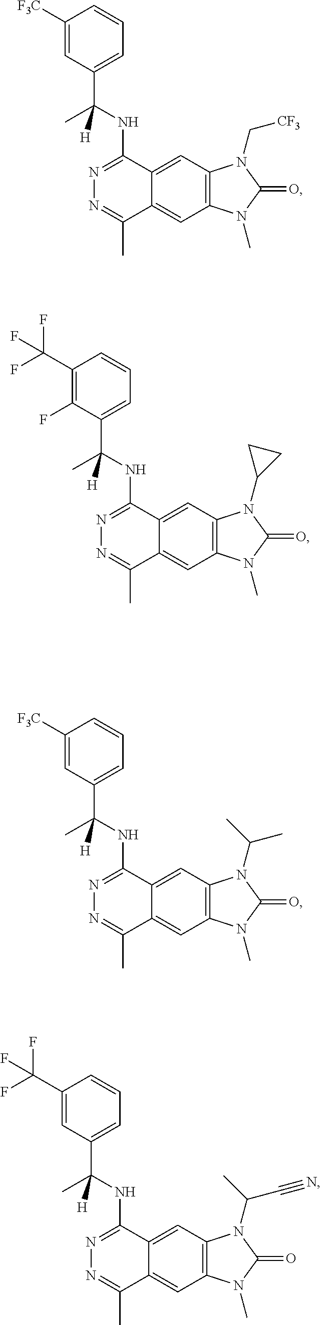 [SMILES: Cc1nnc(N[C@H](C)c2cccc(C(F)(F)F)c2)c2cc3c(cc12)n(C)c(=O)n3CC(F)(F)F ; Cc1nnc(N[C@H](C)c2cccc(C(F)(F)F)c2F)c2cc3c(cc12)n(C)c(=O)n3C1CC1 ; Cc1nnc(N[C@H](C)c2cccc(C(F)(F)F)c2)c2cc3c(cc12)n(C)c(=O)n3C(C)C ; Cc1nnc(N[C@H](C)c2cccc(C(F)(F)F)c2)c2cc3c(cc12)n(C)c(=O)n3C(C)C#N] 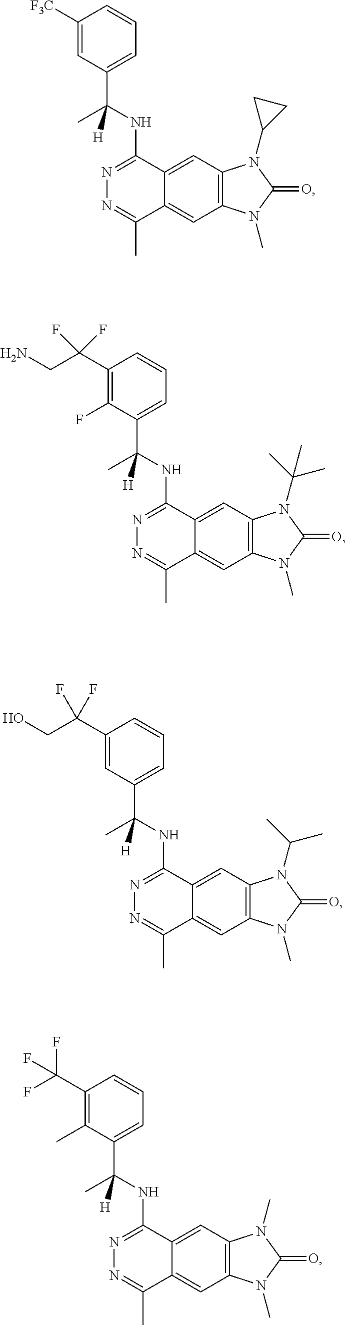 [SMILES: Cc1nnc(N[C@H](C)c2cccc(C(F)(F)F)c2)c2cc3c(cc12)n(C)c(=O)n3C1CC1 ; Cc1nnc(N[C@H](C)c2cccc(C(F)(F)CN)c2F)c2cc3c(cc12)n(C)c(=O)n3C(C)(C)C ; Cc1nnc(N[C@H](C)c2cccc(C(F)(F)CO)c2)c2cc3c(cc12)n(C)c(=O)n3C(C)C ; Cc1c([C@@H](C)Nc2nnc(C)c3cc4c(cc23)n(C)c(=O)n4C)cccc1C(F)(F)F] 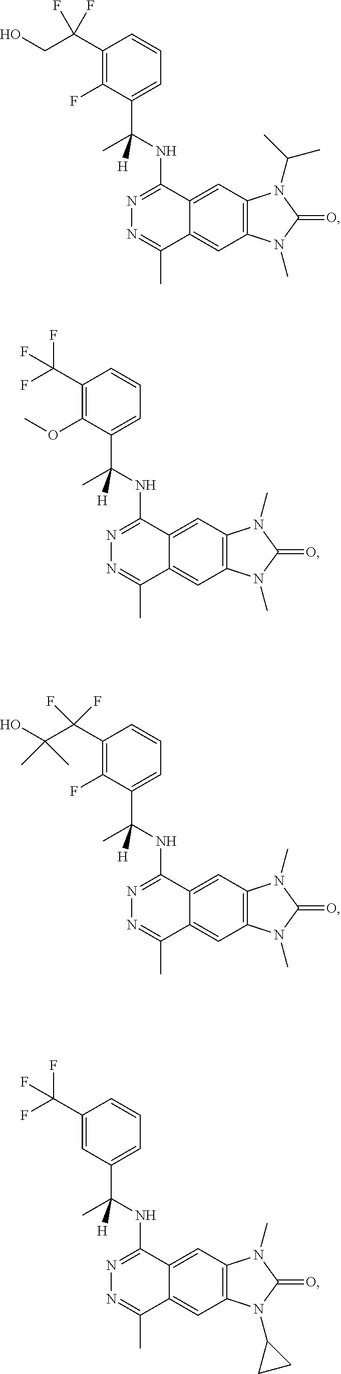 [SMILES: Cc1nnc(N[C@H](C)c2cccc(C(F)(F)CO)c2F)c2cc3c(cc12)n(C)c(=O)n3C(C)C ; COc1c([C@@H](C)Nc2nnc(C)c3cc4c(cc23)n(C)c(=O)n4C)cccc1C(F)(F)F ; Cc1nnc(N[C@H](C)c2cccc(C(F)(F)C(C)(C)O)c2F)c2cc3c(cc12)n(C)c(=O)n3C ; Cc1nnc(N[C@H](C)c2cccc(C(F)(F)F)c2)c2cc3c(cc12)n(C1CC1)c(=O)n3C] 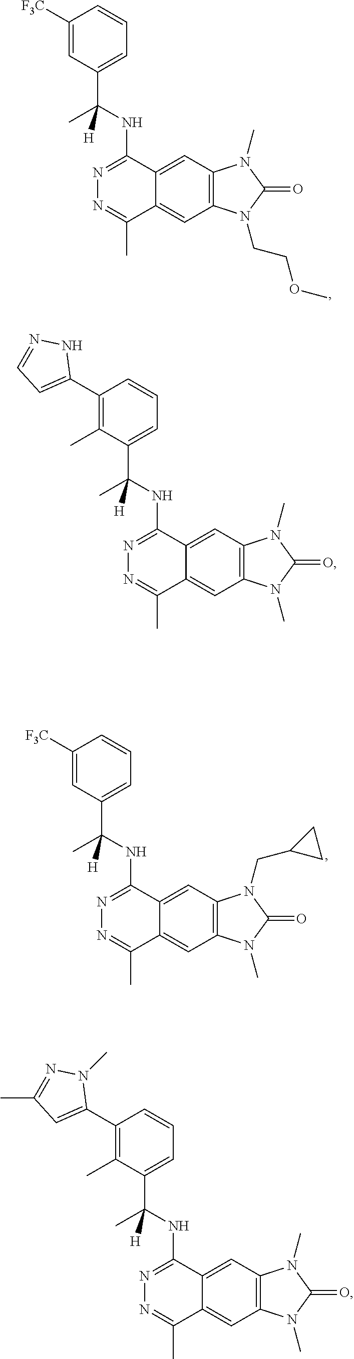 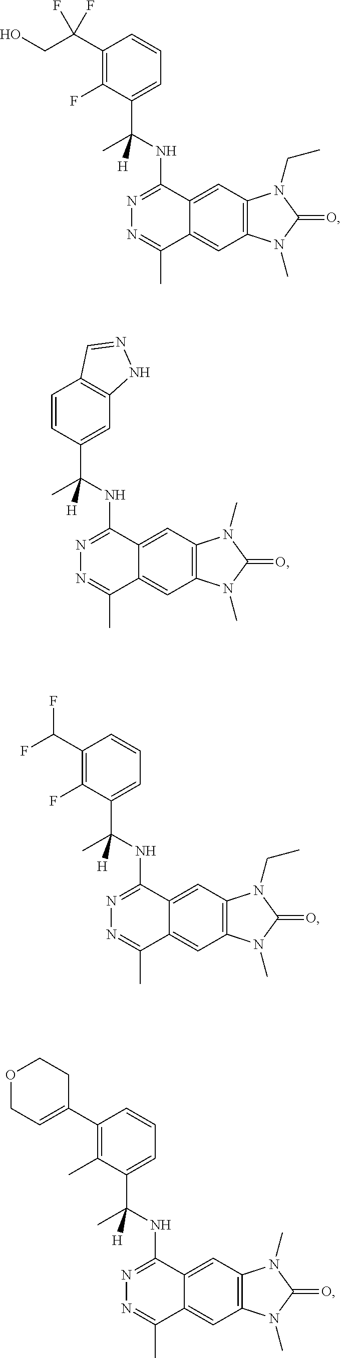 [SMILES: CCn1c(=O)n(C)c2cc3c(C)nnc(N[C@H](C)c4cccc(C(F)(F)CO)c4F)c3cc21 ; Cc1nnc(N[C@H](C)c2ccc3cn[nH]c3c2)c2cc3c(cc12)n(C)c(=O)n3C ; CCn1c(=O)n(C)c2cc3c(C)nnc(N[C@H](C)c4cccc(C(F)F)c4F)c3cc21 ; Cc1c(C2=CCOCC2)cccc1[C@@H](C)Nc1nnc(C)c2cc3c(cc12)n(C)c(=O)n3C] 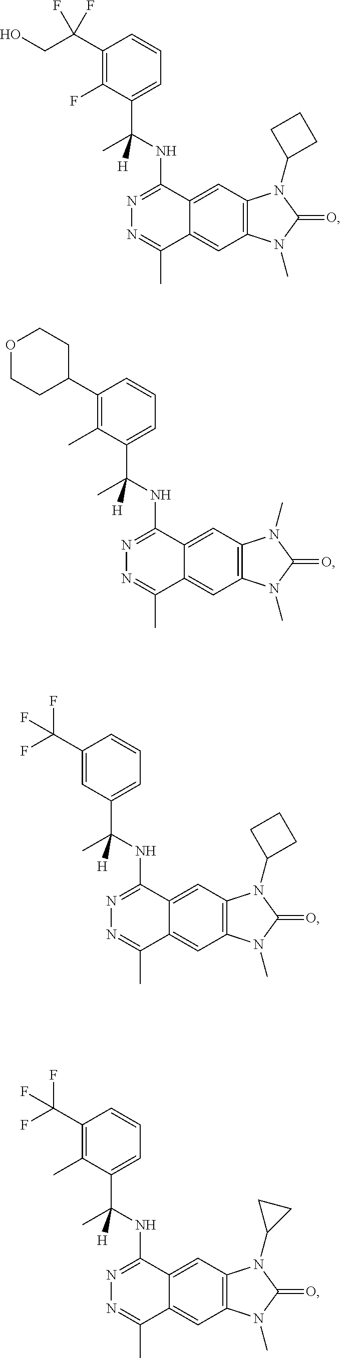 [SMILES: Cc1nnc(N[C@H](C)c2cccc(C(F)(F)CO)c2F)c2cc3c(cc12)n(C)c(=O)n3C1CCC1 ; Cc1c(C2CCOCC2)cccc1[C@@H](C)Nc1nnc(C)c2cc3c(cc12)n(C)c(=O)n3C ; Cc1nnc(N[C@H](C)c2cccc(C(F)(F)F)c2)c2cc3c(cc12)n(C)c(=O)n3C1CCC1 ; Cc1c([C@@H](C)Nc2nnc(C)c3cc4c(cc23)n(C2CC2)c(=O)n4C)cccc1C(F)(F)F] 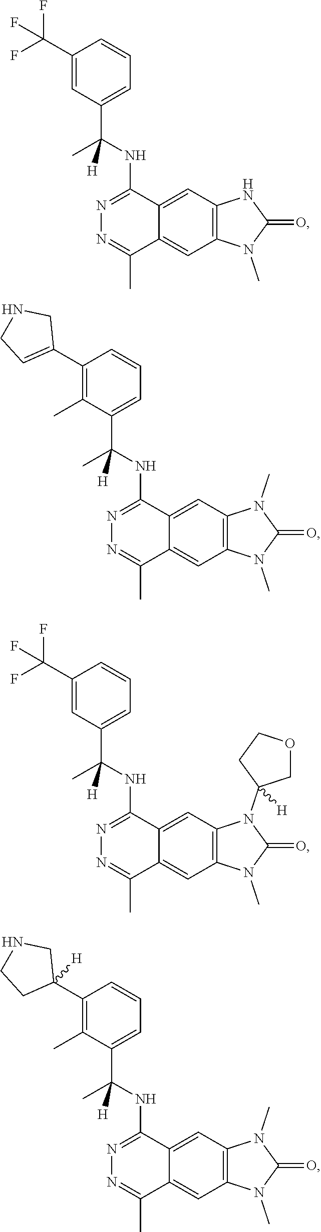 [SMILES: Cc1nnc(N[C@H](C)c2cccc(C(F)(F)F)c2)c2cc3[nH]c(=O)n(C)c3cc12 ; Cc1c(C2=CCNC2)cccc1[C@@H](C)Nc1nnc(C)c2cc3c(cc12)n(C)c(=O)n3C ; Cc1nnc(N[C@H](C)c2cccc(C(F)(F)F)c2)c2cc3c(cc12)n(C)c(=O)n3C1CCOC1 ; Cc1c(C2CCNC2)cccc1[C@@H](C)Nc1nnc(C)c2cc3c(cc12)n(C)c(=O)n3C] 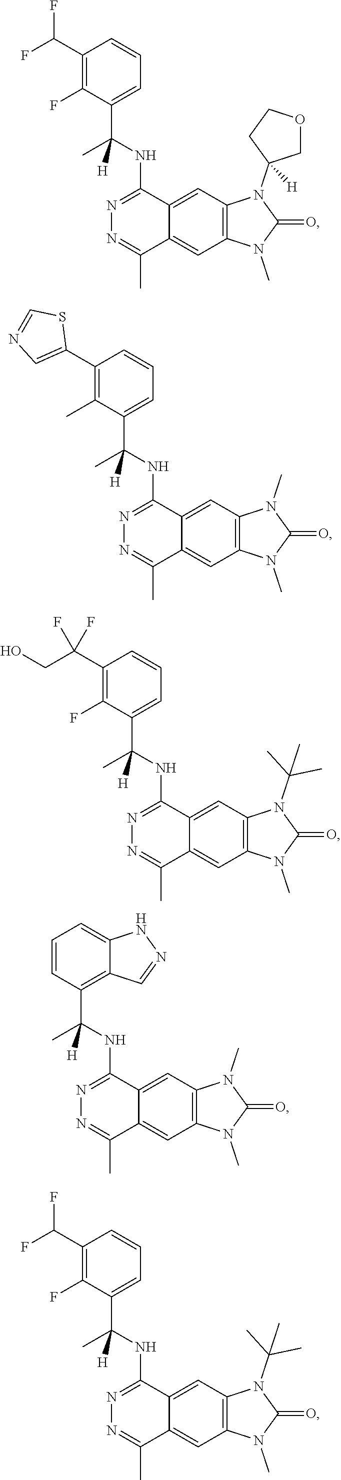 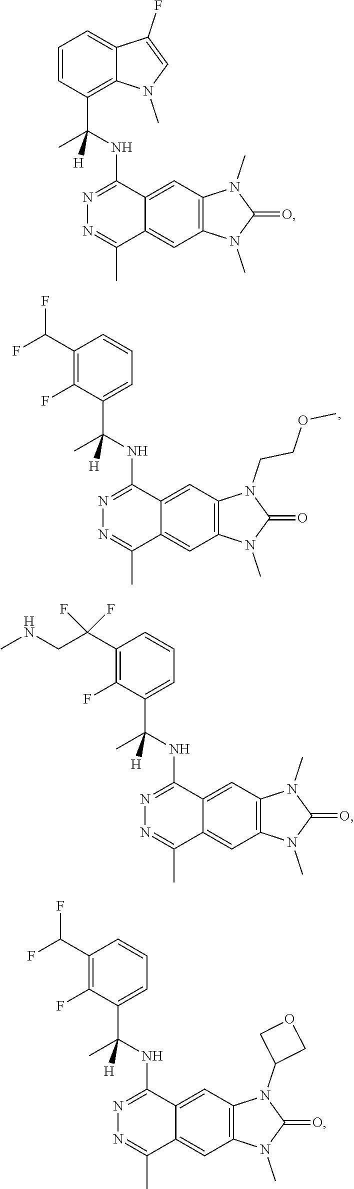 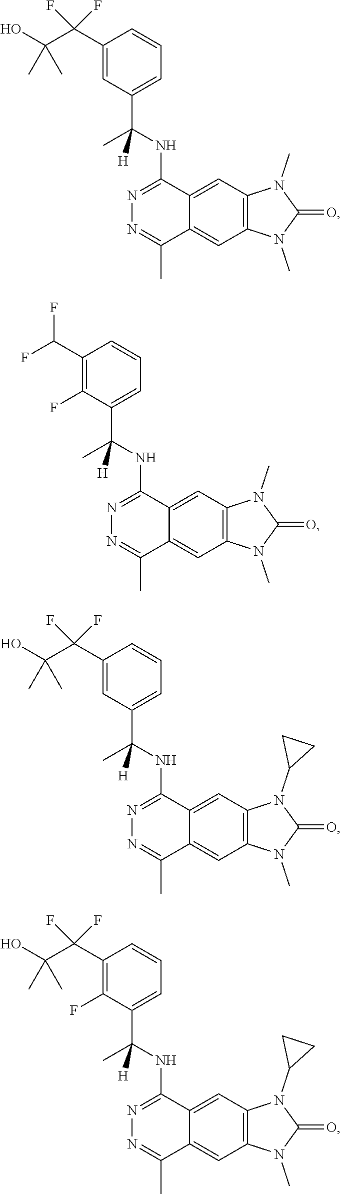 [SMILES: Cc1nnc(N[C@H](C)c2cccc(C(F)(F)C(C)(C)O)c2)c2cc3c(cc12)n(C)c(=O)n3C ; Cc1nnc(N[C@H](C)c2cccc(C(F)F)c2F)c2cc3c(cc12)n(C)c(=O)n3C ; Cc1nnc(N[C@H](C)c2cccc(C(F)(F)C(C)(C)O)c2)c2cc3c(cc12)n(C)c(=O)n3C1CC1 ; Cc1nnc(N[C@H](C)c2cccc(C(F)(F)C(C)(C)O)c2F)c2cc3c(cc12)n(C)c(=O)n3C1CC1] 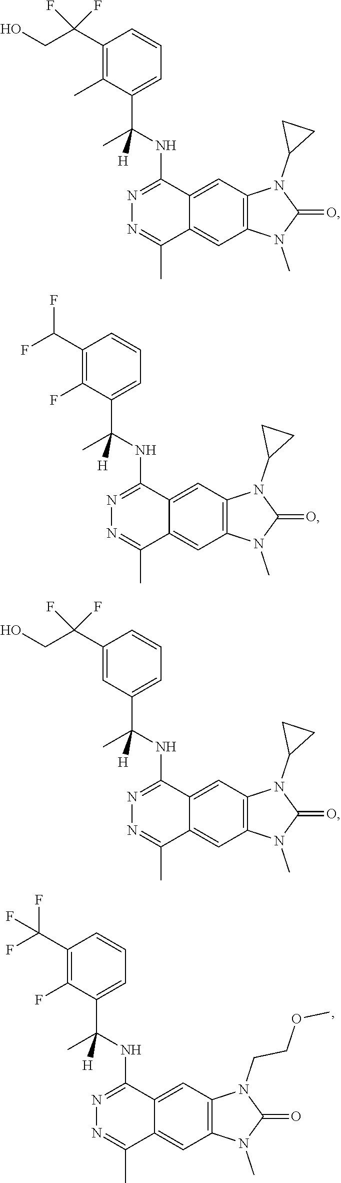 [SMILES: Cc1c([C@@H](C)Nc2nnc(C)c3cc4c(cc23)n(C2CC2)c(=O)n4C)cccc1C(F)(F)CO ; Cc1nnc(N[C@H](C)c2cccc(C(F)F)c2F)c2cc3c(cc12)n(C)c(=O)n3C1CC1 ; Cc1nnc(N[C@H](C)c2cccc(C(F)(F)CO)c2)c2cc3c(cc12)n(C)c(=O)n3C1CC1 ; COCCn1c(=O)n(C)c2cc3c(C)nnc(N[C@H](C)c4cccc(C(F)(F)F)c4F)c3cc21] 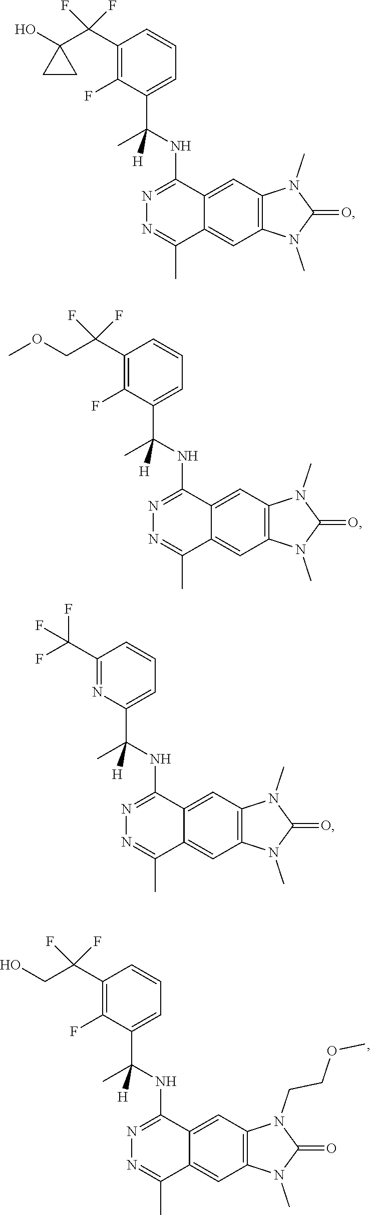 [SMILES: Cc1nnc(N[C@H](C)c2cccc(C(F)(F)C3(O)CC3)c2F)c2cc3c(cc12)n(C)c(=O)n3C ; COCC(F)(F)c1cccc([C@@H](C)Nc2nnc(C)c3cc4c(cc23)n(C)c(=O)n4C)c1F ; Cc1nnc(N[C@H](C)c2cccc(C(F)(F)F)n2)c2cc3c(cc12)n(C)c(=O)n3C ; COCCn1c(=O)n(C)c2cc3c(C)nnc(N[C@H](C)c4cccc(C(F)(F)CO)c4F)c3cc21] 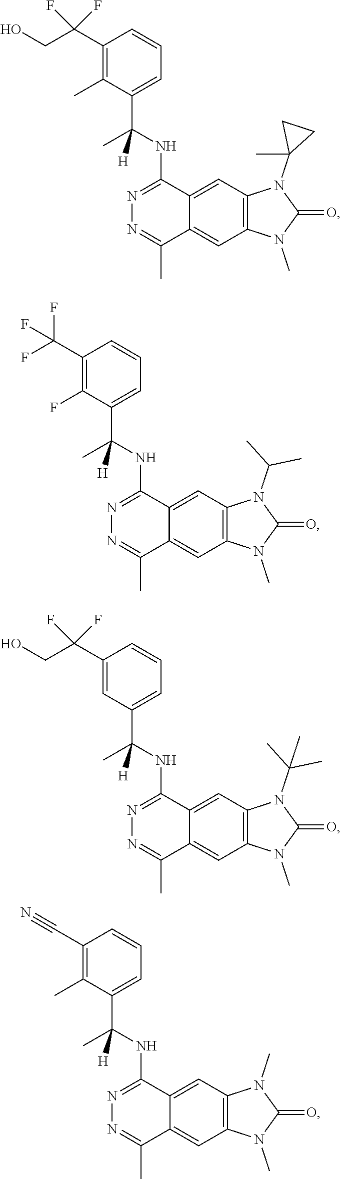 [SMILES: Cc1c([C@@H](C)Nc2nnc(C)c3cc4c(cc23)n(C2(C)CC2)c(=O)n4C)cccc1C(F)(F)CO ; Cc1nnc(N[C@H](C)c2cccc(C(F)(F)F)c2F)c2cc3c(cc12)n(C)c(=O)n3C(C)C ; Cc1nnc(N[C@H](C)c2cccc(C(F)(F)CO)c2)c2cc3c(cc12)n(C)c(=O)n3C(C)(C)C ; Cc1c(C#N)cccc1[C@@H](C)Nc1nnc(C)c2cc3c(cc12)n(C)c(=O)n3C] 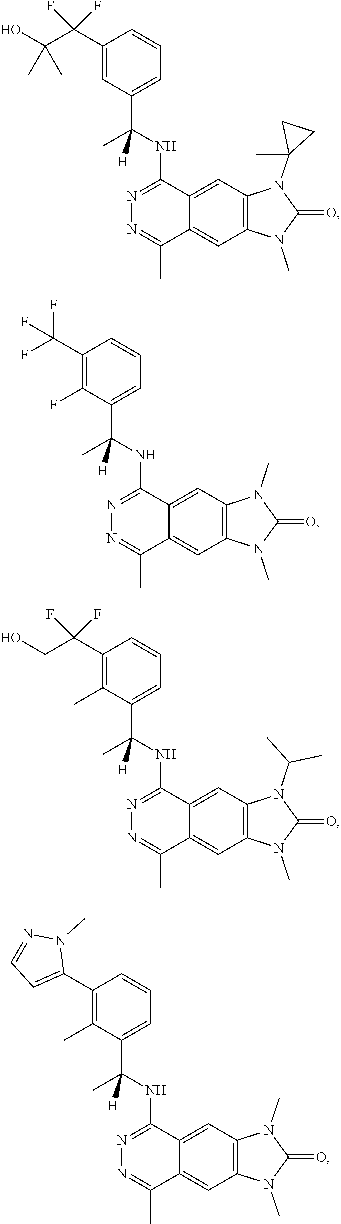 [SMILES: Cc1nnc(N[C@H](C)c2cccc(C(F)(F)C(C)(C)O)c2)c2cc3c(cc12)n(C)c(=O)n3C1(C)CC1 ; Cc1nnc(N[C@H](C)c2cccc(C(F)(F)F)c2F)c2cc3c(cc12)n(C)c(=O)n3C ; Cc1c([C@@H](C)Nc2nnc(C)c3cc4c(cc23)n(C(C)C)c(=O)n4C)cccc1C(F)(F)CO ; Cc1c(-c2ccnn2C)cccc1[C@@H](C)Nc1nnc(C)c2cc3c(cc12)n(C)c(=O)n3C] 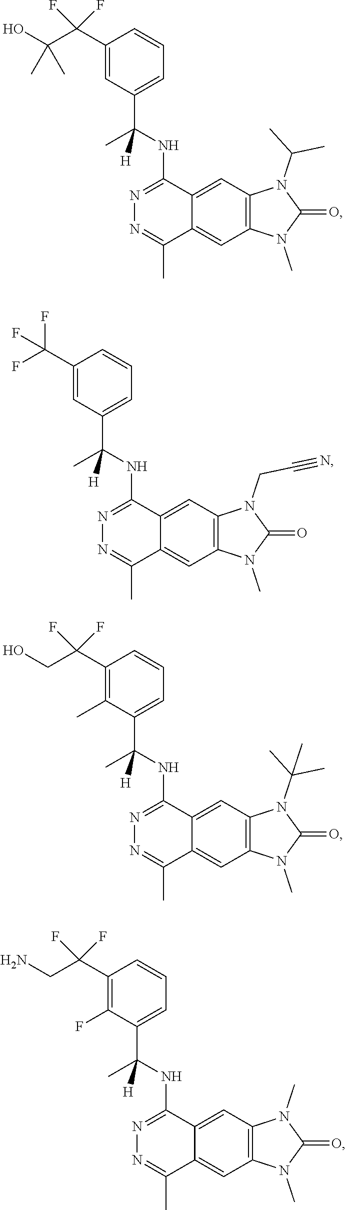 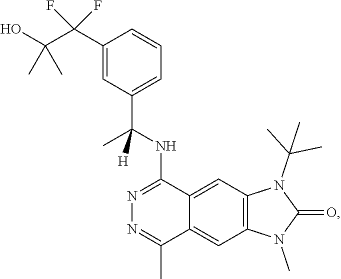 [SMILES: Cc1nnc(N[C@H](C)c2cccc(C(F)(F)C(C)(C)O)c2)c2cc3c(cc12)n(C)c(=O)n3C(C)(C)C]
or a pharmaceutically acceptable salt thereof.

54. The compound of claim 1, wherein the compound is: 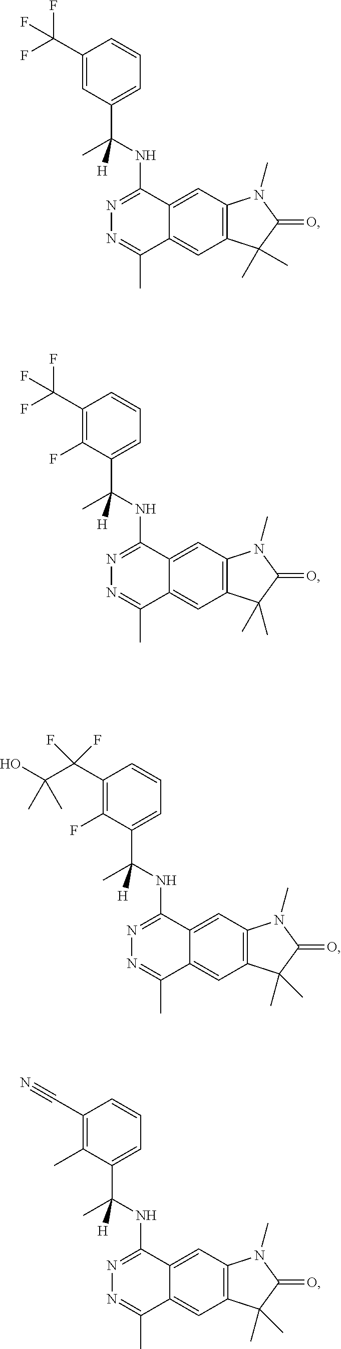 [SMILES: Cc1nnc(N[C@H](C)c2cccc(C(F)(F)F)c2)c2cc3c(cc12)C(C)(C)C(=O)N3C ; Cc1nnc(N[C@H](C)c2cccc(C(F)(F)F)c2F)c2cc3c(cc12)C(C)(C)C(=O)N3C ; Cc1nnc(N[C@H](C)c2cccc(C(F)(F)C(C)(C)O)c2F)c2cc3c(cc12)C(C)(C)C(=O)N3C ; Cc1c(C#N)cccc1[C@@H](C)Nc1nnc(C)c2cc3c(cc12)N(C)C(=O)C3(C)C] 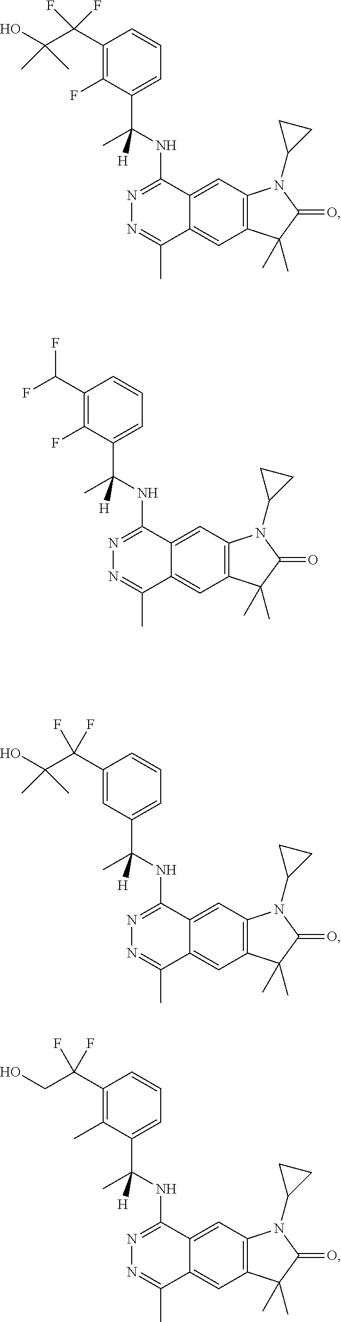 [SMILES: Cc1nnc(N[C@H](C)c2cccc(C(F)(F)C(C)(C)O)c2F)c2cc3c(cc12)C(C)(C)C(=O)N3C1CC1 ; Cc1nnc(N[C@H](C)c2cccc(C(F)F)c2F)c2cc3c(cc12)C(C)(C)C(=O)N3C1CC1 ; Cc1nnc(N[C@H](C)c2cccc(C(F)(F)C(C)(C)O)c2)c2cc3c(cc12)C(C)(C)C(=O)N3C1CC1 ; Cc1c([C@@H](C)Nc2nnc(C)c3cc4c(cc23)N(C2CC2)C(=O)C4(C)C)cccc1C(F)(F)CO] 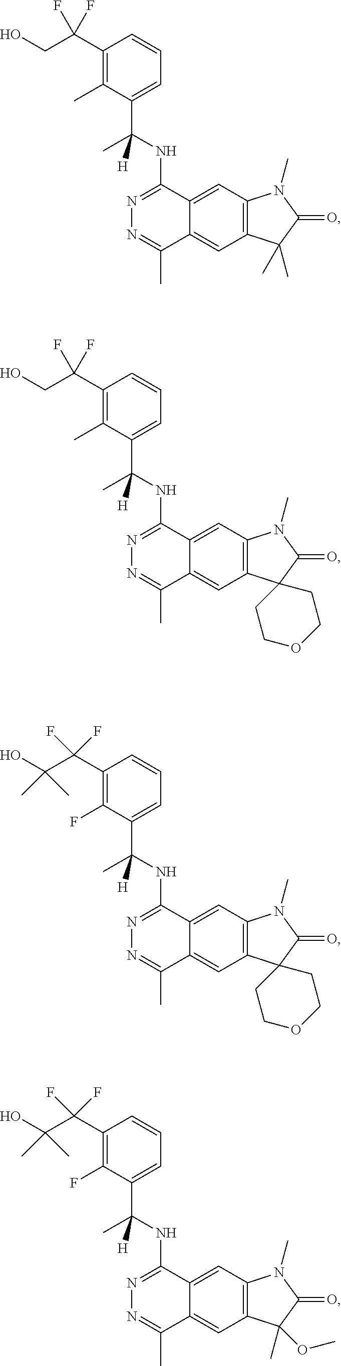 [SMILES: Cc1c([C@@H](C)Nc2nnc(C)c3cc4c(cc23)N(C)C(=O)C4(C)C)cccc1C(F)(F)CO ; Cc1c([C@@H](C)Nc2nnc(C)c3cc4c(cc23)N(C)C(=O)C42CCOCC2)cccc1C(F)(F)CO ; Cc1nnc(N[C@H](C)c2cccc(C(F)(F)C(C)(C)O)c2F)c2cc3c(cc12)C1(CCOCC1)C(=O)N3C ; COC1(C)C(=O)N(C)c2cc3c(N[C@H](C)c4cccc(C(F)(F)C(C)(C)O)c4F)nnc(C)c3cc21] 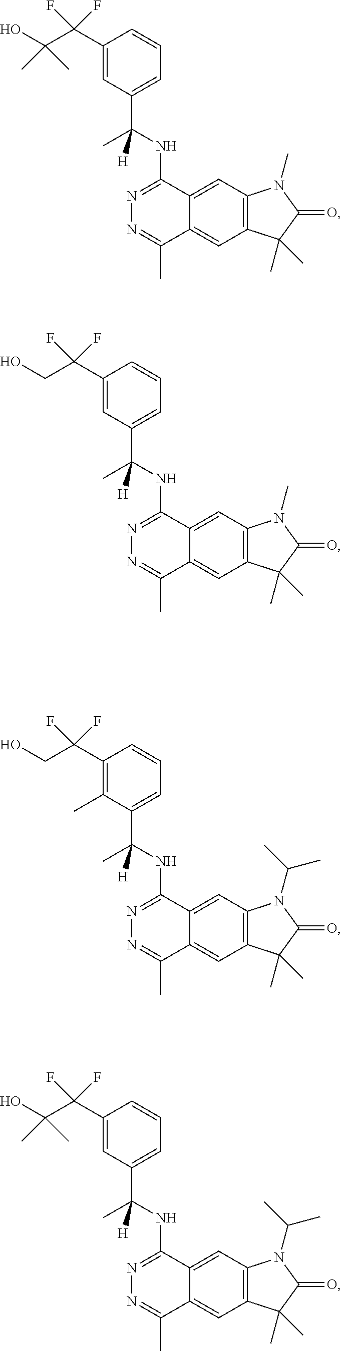 [SMILES: Cc1nnc(N[C@H](C)c2cccc(C(F)(F)C(C)(C)O)c2)c2cc3c(cc12)C(C)(C)C(=O)N3C ; Cc1nnc(N[C@H](C)c2cccc(C(F)(F)CO)c2)c2cc3c(cc12)C(C)(C)C(=O)N3C ; Cc1c([C@@H](C)Nc2nnc(C)c3cc4c(cc23)N(C(C)C)C(=O)C4(C)C)cccc1C(F)(F)CO ; Cc1nnc(N[C@H](C)c2cccc(C(F)(F)C(C)(C)O)c2)c2cc3c(cc12)C(C)(C)C(=O)N3C(C)C] 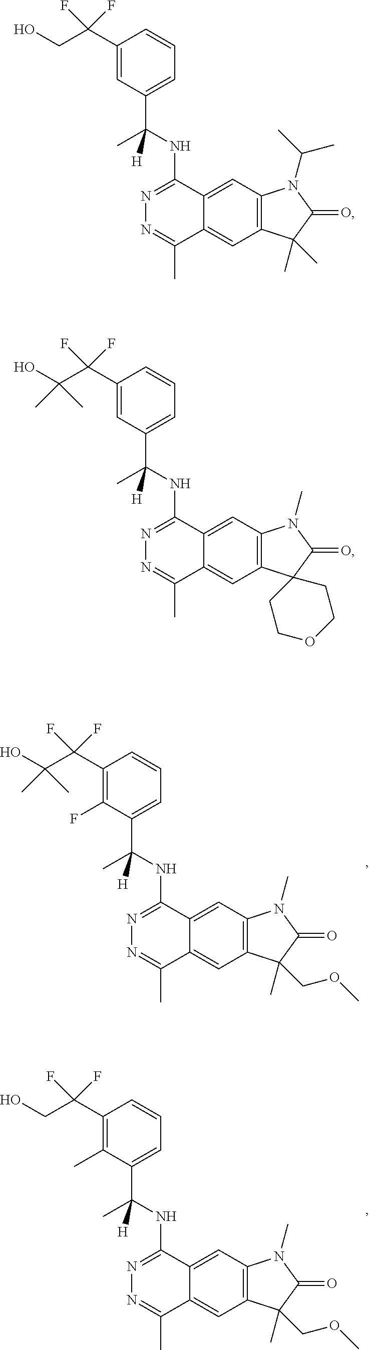 [SMILES: Cc1nnc(N[C@H](C)c2cccc(C(F)(F)CO)c2)c2cc3c(cc12)C(C)(C)C(=O)N3C(C)C ; Cc1nnc(N[C@H](C)c2cccc(C(F)(F)C(C)(C)O)c2)c2cc3c(cc12)C1(CCOCC1)C(=O)N3C ; COCC1(C)C(=O)N(C)c2cc3c(N[C@H](C)c4cccc(C(F)(F)C(C)(C)O)c4F)nnc(C)c3cc21 ; COCC1(C)C(=O)N(C)c2cc3c(N[C@H](C)c4cccc(C(F)(F)CO)c4C)nnc(C)c3cc21] 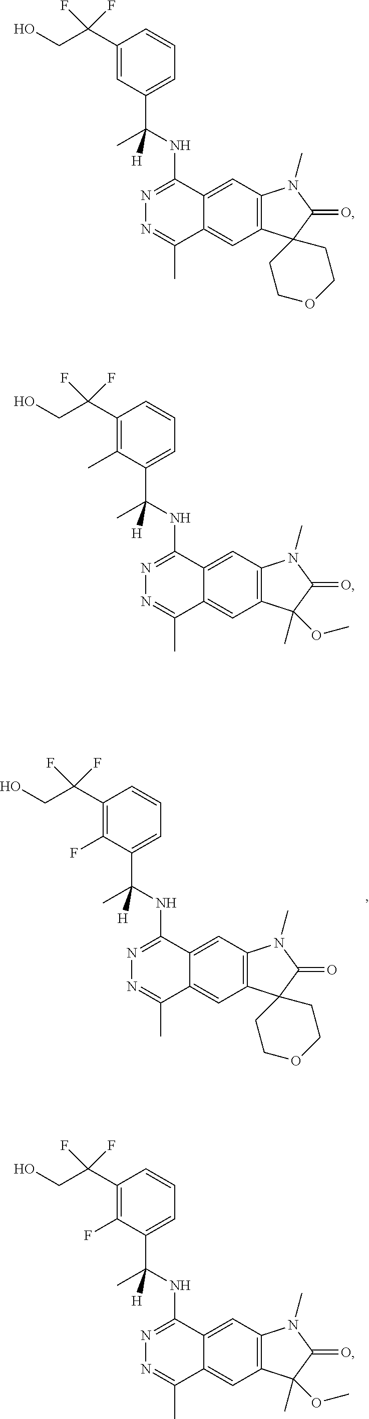 [SMILES: Cc1nnc(N[C@H](C)c2cccc(C(F)(F)CO)c2)c2cc3c(cc12)C1(CCOCC1)C(=O)N3C ; COC1(C)C(=O)N(C)c2cc3c(N[C@H](C)c4cccc(C(F)(F)CO)c4C)nnc(C)c3cc21 ; Cc1nnc(N[C@H](C)c2cccc(C(F)(F)CO)c2F)c2cc3c(cc12)C1(CCOCC1)C(=O)N3C ; COC1(C)C(=O)N(C)c2cc3c(N[C@H](C)c4cccc(C(F)(F)CO)c4F)nnc(C)c3cc21] 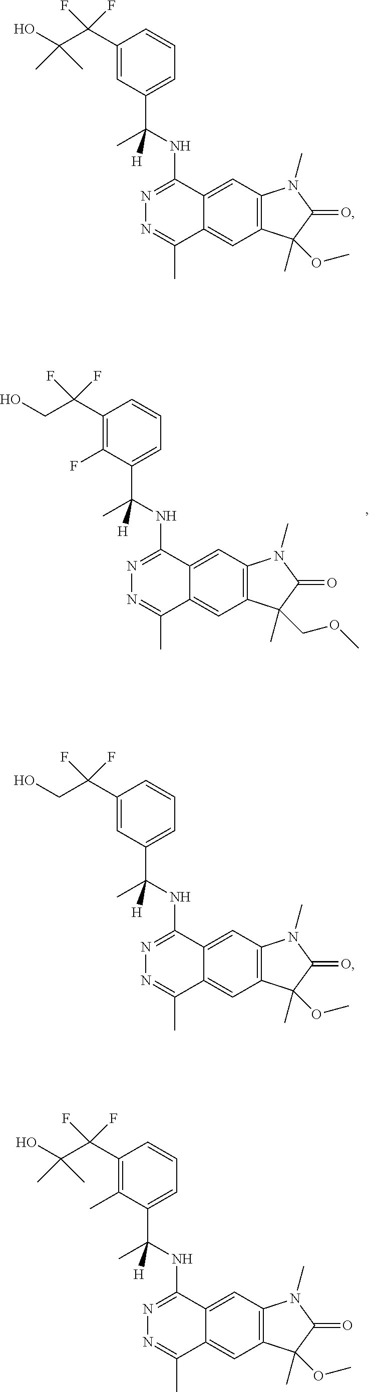 [SMILES: COC1(C)C(=O)N(C)c2cc3c(N[C@H](C)c4cccc(C(F)(F)C(C)(C)O)c4)nnc(C)c3cc21 ; COCC1(C)C(=O)N(C)c2cc3c(N[C@H](C)c4cccc(C(F)(F)CO)c4F)nnc(C)c3cc21 ; COC1(C)C(=O)N(C)c2cc3c(N[C@H](C)c4cccc(C(F)(F)CO)c4)nnc(C)c3cc21 ; COC1(C)C(=O)N(C)c2cc3c(N[C@H](C)c4cccc(C(F)(F)C(C)(C)O)c4C)nnc(C)c3cc21] 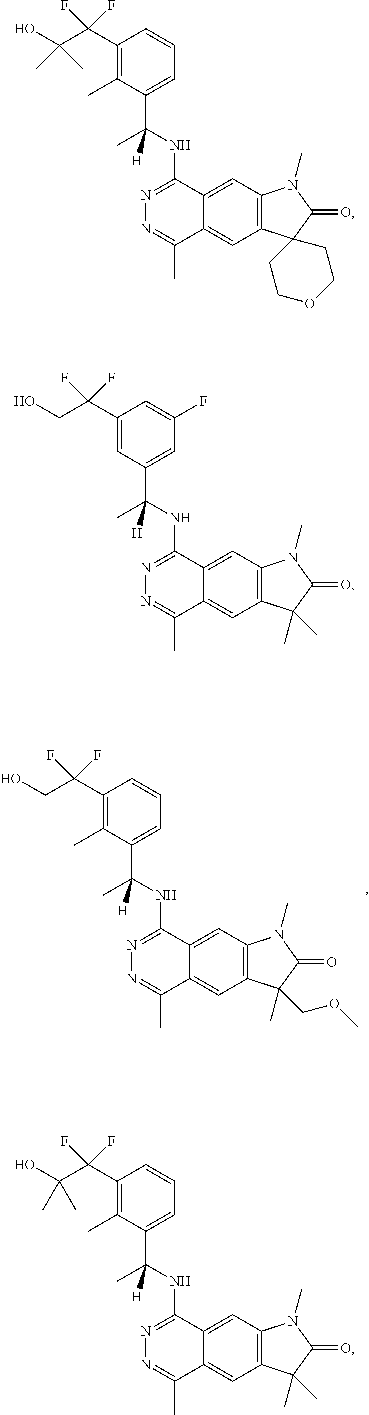 [SMILES: Cc1c([C@@H](C)Nc2nnc(C)c3cc4c(cc23)N(C)C(=O)C42CCOCC2)cccc1C(F)(F)C(C)(C)O ; Cc1nnc(N[C@H](C)c2cc(F)cc(C(F)(F)CO)c2)c2cc3c(cc12)C(C)(C)C(=O)N3C ; COCC1(C)C(=O)N(C)c2cc3c(N[C@H](C)c4cccc(C(F)(F)CO)c4C)nnc(C)c3cc21 ; Cc1c([C@@H](C)Nc2nnc(C)c3cc4c(cc23)N(C)C(=O)C4(C)C)cccc1C(F)(F)C(C)(C)O] 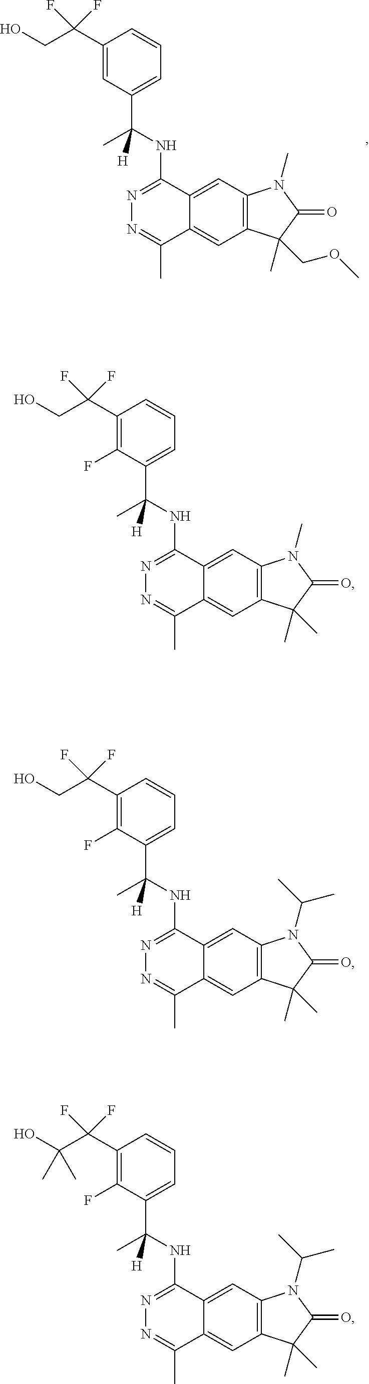 [SMILES: COCC1(C)C(=O)N(C)c2cc3c(N[C@H](C)c4cccc(C(F)(F)CO)c4)nnc(C)c3cc21 ; Cc1nnc(N[C@H](C)c2cccc(C(F)(F)CO)c2F)c2cc3c(cc12)C(C)(C)C(=O)N3C ; Cc1nnc(N[C@H](C)c2cccc(C(F)(F)CO)c2F)c2cc3c(cc12)C(C)(C)C(=O)N3C(C)C ; Cc1nnc(N[C@H](C)c2cccc(C(F)(F)C(C)(C)O)c2F)c2cc3c(cc12)C(C)(C)C(=O)N3C(C)C] 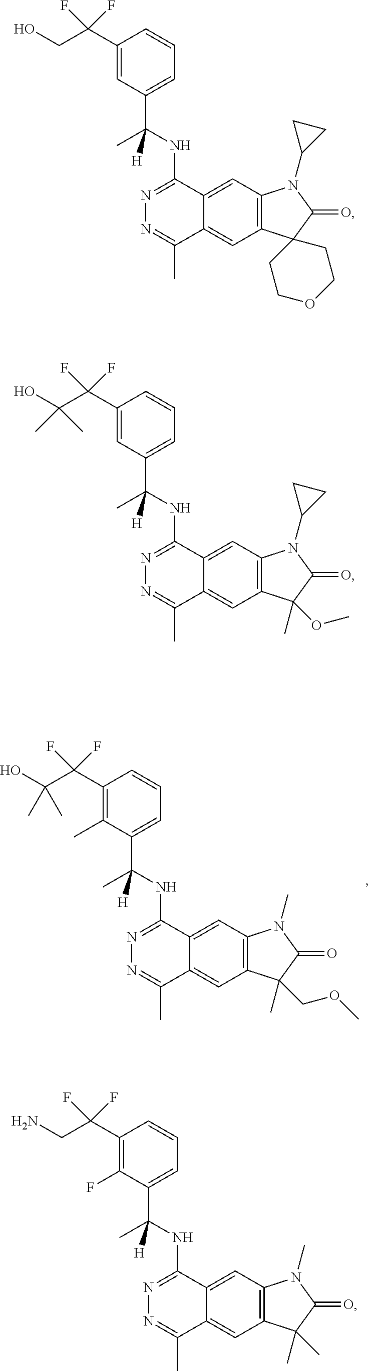 [SMILES: Cc1nnc(N[C@H](C)c2cccc(C(F)(F)CO)c2)c2cc3c(cc12)C1(CCOCC1)C(=O)N3C1CC1 ; COC1(C)C(=O)N(C2CC2)c2cc3c(N[C@H](C)c4cccc(C(F)(F)C(C)(C)O)c4)nnc(C)c3cc21 ; COCC1(C)C(=O)N(C)c2cc3c(N[C@H](C)c4cccc(C(F)(F)C(C)(C)O)c4C)nnc(C)c3cc21 ; Cc1nnc(N[C@H](C)c2cccc(C(F)(F)CN)c2F)c2cc3c(cc12)C(C)(C)C(=O)N3C] 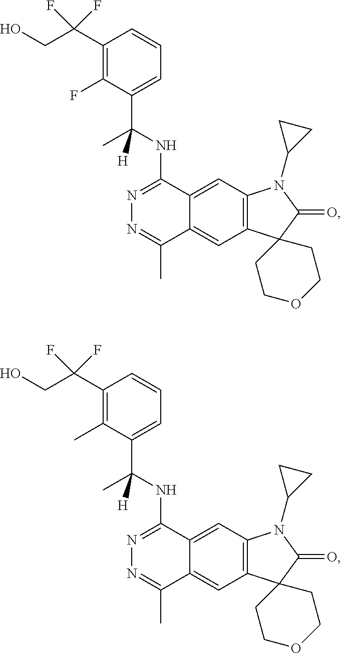 [SMILES: Cc1nnc(N[C@H](C)c2cccc(C(F)(F)CO)c2F)c2cc3c(cc12)C1(CCOCC1)C(=O)N3C1CC1 ; Cc1c([C@@H](C)Nc2nnc(C)c3cc4c(cc23)N(C2CC2)C(=O)C42CCOCC2)cccc1C(F)(F)CO] 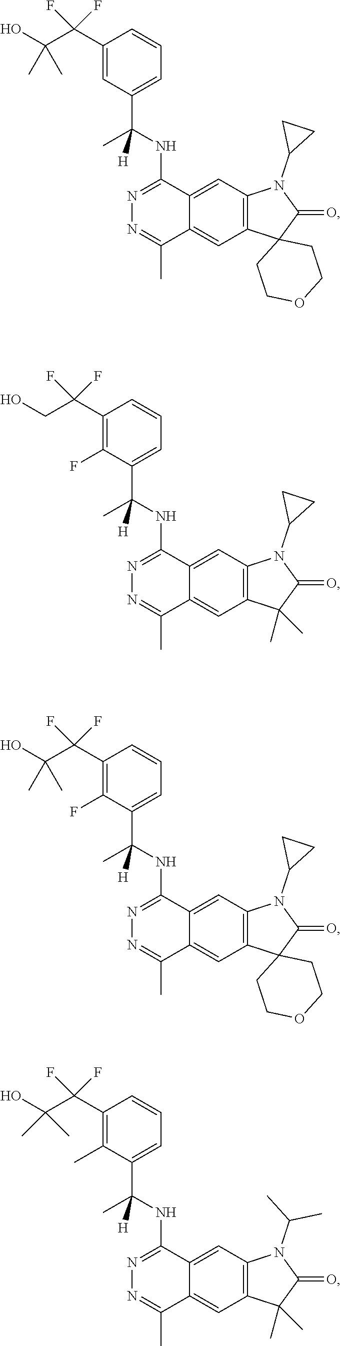 [SMILES: Cc1nnc(N[C@H](C)c2cccc(C(F)(F)C(C)(C)O)c2)c2cc3c(cc12)C1(CCOCC1)C(=O)N3C1CC1 ; Cc1nnc(N[C@H](C)c2cccc(C(F)(F)CO)c2F)c2cc3c(cc12)C(C)(C)C(=O)N3C1CC1 ; Cc1nnc(N[C@H](C)c2cccc(C(F)(F)C(C)(C)O)c2F)c2cc3c(cc12)C1(CCOCC1)C(=O)N3C1CC1 ; Cc1c([C@@H](C)Nc2nnc(C)c3cc4c(cc23)N(C(C)C)C(=O)C4(C)C)cccc1C(F)(F)C(C)(C)O] 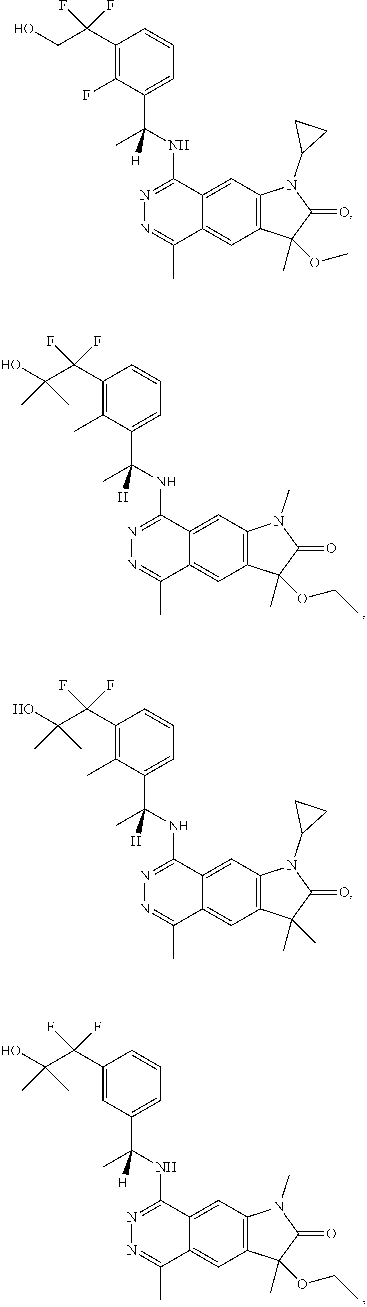 [SMILES: COC1(C)C(=O)N(C2CC2)c2cc3c(N[C@H](C)c4cccc(C(F)(F)CO)c4F)nnc(C)c3cc21 ; CCOC1(C)C(=O)N(C)c2cc3c(N[C@H](C)c4cccc(C(F)(F)C(C)(C)O)c4C)nnc(C)c3cc21 ; Cc1c([C@@H](C)Nc2nnc(C)c3cc4c(cc23)N(C2CC2)C(=O)C4(C)C)cccc1C(F)(F)C(C)(C)O ; CCOC1(C)C(=O)N(C)c2cc3c(N[C@H](C)c4cccc(C(F)(F)C(C)(C)O)c4)nnc(C)c3cc21] 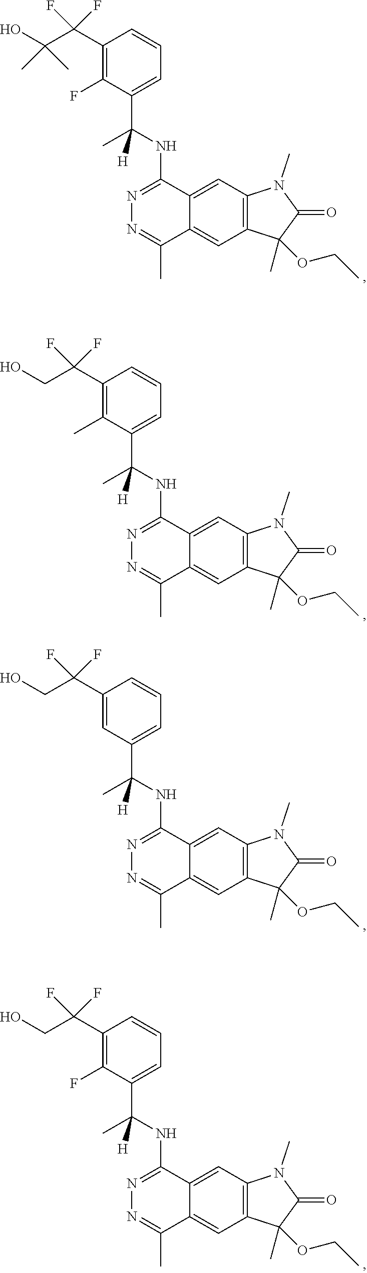 [SMILES: CCOC1(C)C(=O)N(C)c2cc3c(N[C@H](C)c4cccc(C(F)(F)C(C)(C)O)c4F)nnc(C)c3cc21 ; CCOC1(C)C(=O)N(C)c2cc3c(N[C@H](C)c4cccc(C(F)(F)CO)c4C)nnc(C)c3cc21 ; CCOC1(C)C(=O)N(C)c2cc3c(N[C@H](C)c4cccc(C(F)(F)CO)c4)nnc(C)c3cc21 ; CCOC1(C)C(=O)N(C)c2cc3c(N[C@H](C)c4cccc(C(F)(F)CO)c4F)nnc(C)c3cc21] 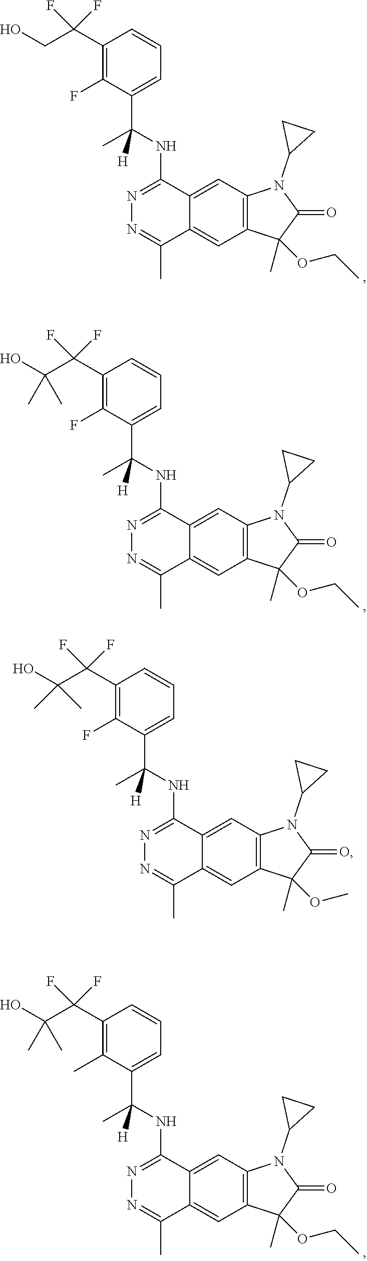 [SMILES: CCOC1(C)C(=O)N(C2CC2)c2cc3c(N[C@H](C)c4cccc(C(F)(F)CO)c4F)nnc(C)c3cc21 ; CCOC1(C)C(=O)N(C2CC2)c2cc3c(N[C@H](C)c4cccc(C(F)(F)C(C)(C)O)c4F)nnc(C)c3cc21 ; COC1(C)C(=O)N(C2CC2)c2cc3c(N[C@H](C)c4cccc(C(F)(F)C(C)(C)O)c4F)nnc(C)c3cc21 ; CCOC1(C)C(=O)N(C2CC2)c2cc3c(N[C@H](C)c4cccc(C(F)(F)C(C)(C)O)c4C)nnc(C)c3cc21] 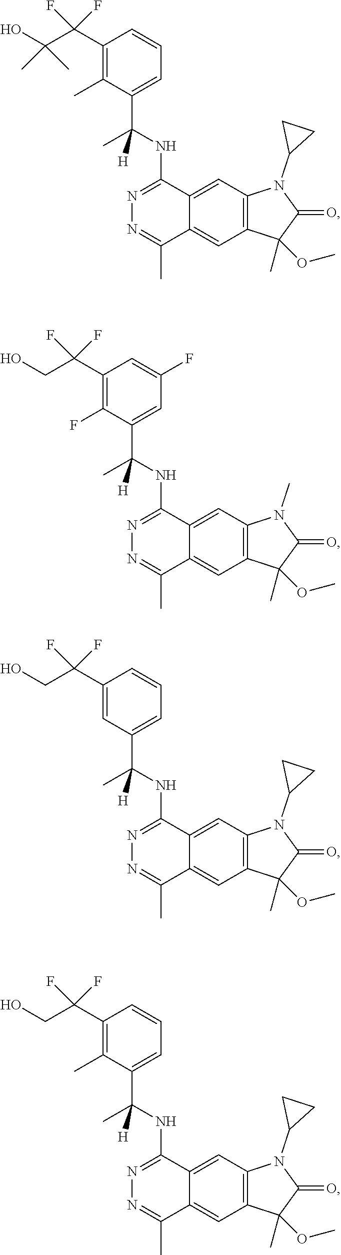 [SMILES: COC1(C)C(=O)N(C2CC2)c2cc3c(N[C@H](C)c4cccc(C(F)(F)C(C)(C)O)c4C)nnc(C)c3cc21 ; COC1(C)C(=O)N(C)c2cc3c(N[C@H](C)c4cc(F)cc(C(F)(F)CO)c4F)nnc(C)c3cc21 ; COC1(C)C(=O)N(C2CC2)c2cc3c(N[C@H](C)c4cccc(C(F)(F)CO)c4)nnc(C)c3cc21 ; COC1(C)C(=O)N(C2CC2)c2cc3c(N[C@H](C)c4cccc(C(F)(F)CO)c4C)nnc(C)c3cc21]
or a pharmaceutically acceptable salt thereof.

55. The compound of claim 1, wherein the compound is: 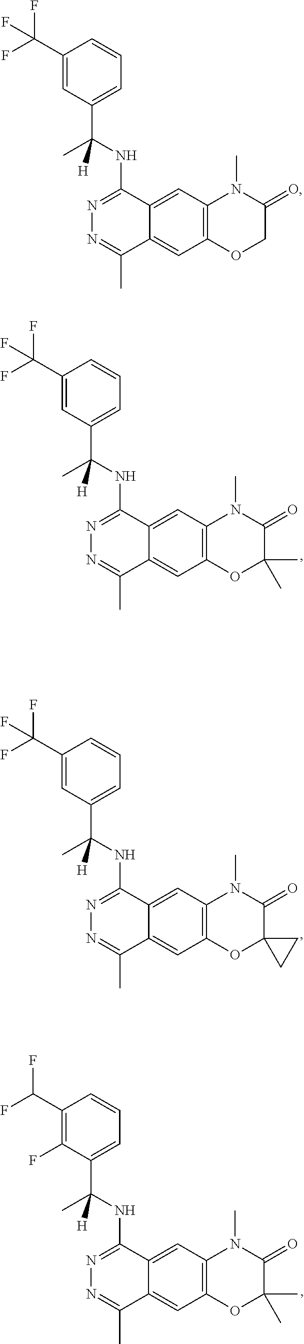 [SMILES: Cc1nnc(N[C@H](C)c2cccc(C(F)(F)F)c2)c2cc3c(cc12)OCC(=O)N3C ; Cc1nnc(N[C@H](C)c2cccc(C(F)(F)F)c2)c2cc3c(cc12)OC(C)(C)C(=O)N3C ; Cc1nnc(N[C@H](C)c2cccc(C(F)(F)F)c2)c2cc3c(cc12)OC1(CC1)C(=O)N3C ; Cc1nnc(N[C@H](C)c2cccc(C(F)F)c2F)c2cc3c(cc12)OC(C)(C)C(=O)N3C] 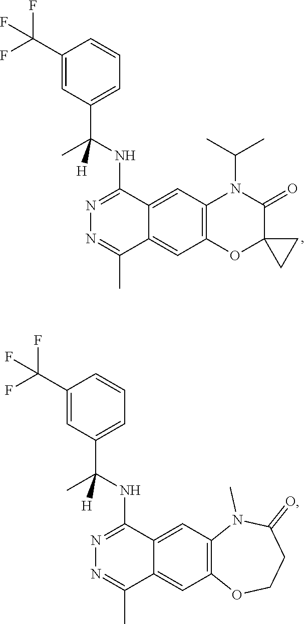 [SMILES: Cc1nnc(N[C@H](C)c2cccc(C(F)(F)F)c2)c2cc3c(cc12)OC1(CC1)C(=O)N3C(C)C ; Cc1nnc(N[C@H](C)c2cccc(C(F)(F)F)c2)c2cc3c(cc12)OCCC(=O)N3C]
or a pharmaceutically acceptable salt thereof.

56. The compound of claim 1, wherein the compound is: 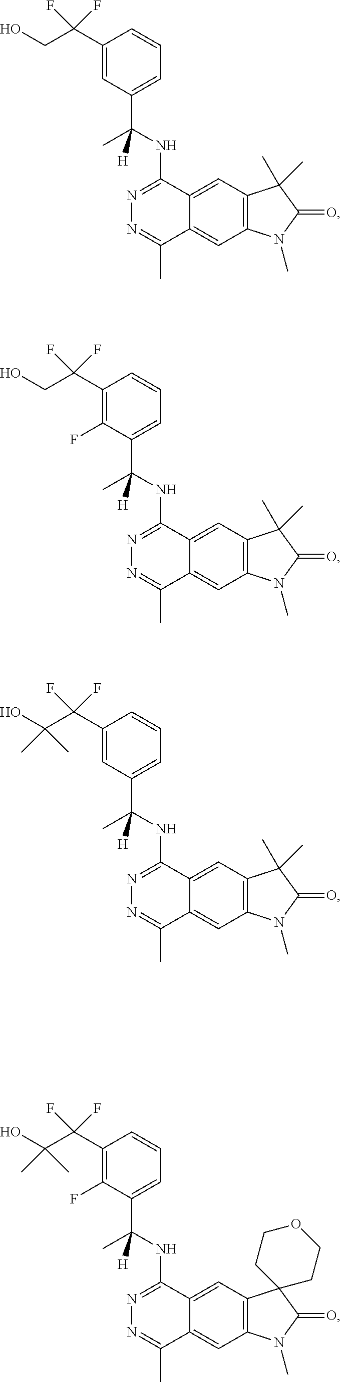 [SMILES: Cc1nnc(N[C@H](C)c2cccc(C(F)(F)CO)c2)c2cc3c(cc12)N(C)C(=O)C3(C)C ; Cc1nnc(N[C@H](C)c2cccc(C(F)(F)CO)c2F)c2cc3c(cc12)N(C)C(=O)C3(C)C ; Cc1nnc(N[C@H](C)c2cccc(C(F)(F)C(C)(C)O)c2)c2cc3c(cc12)N(C)C(=O)C3(C)C ; Cc1nnc(N[C@H](C)c2cccc(C(F)(F)C(C)(C)O)c2F)c2cc3c(cc12)N(C)C(=O)C31CCOCC1] 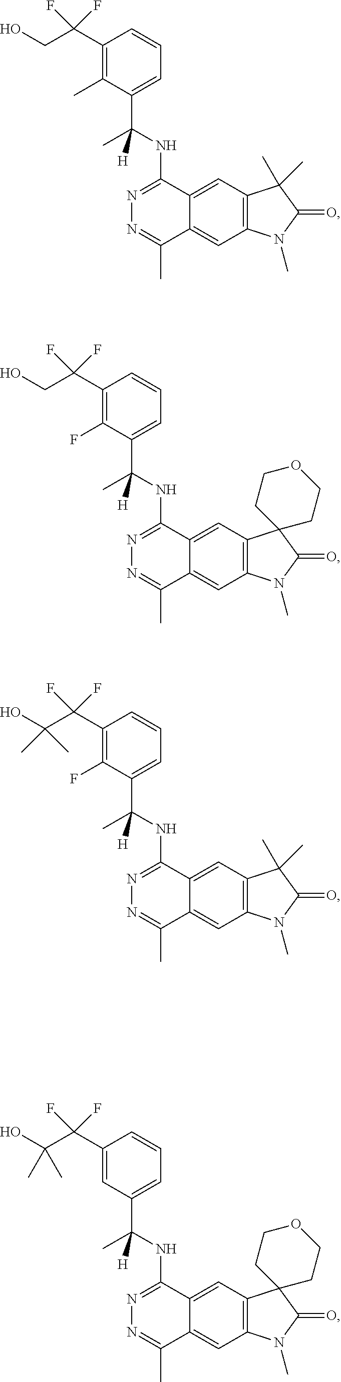 [SMILES: Cc1c([C@@H](C)Nc2nnc(C)c3cc4c(cc23)C(C)(C)C(=O)N4C)cccc1C(F)(F)CO ; Cc1nnc(N[C@H](C)c2cccc(C(F)(F)CO)c2F)c2cc3c(cc12)N(C)C(=O)C31CCOCC1 ; Cc1nnc(N[C@H](C)c2cccc(C(F)(F)C(C)(C)O)c2F)c2cc3c(cc12)N(C)C(=O)C3(C)C ; Cc1nnc(N[C@H](C)c2cccc(C(F)(F)C(C)(C)O)c2)c2cc3c(cc12)N(C)C(=O)C31CCOCC1] 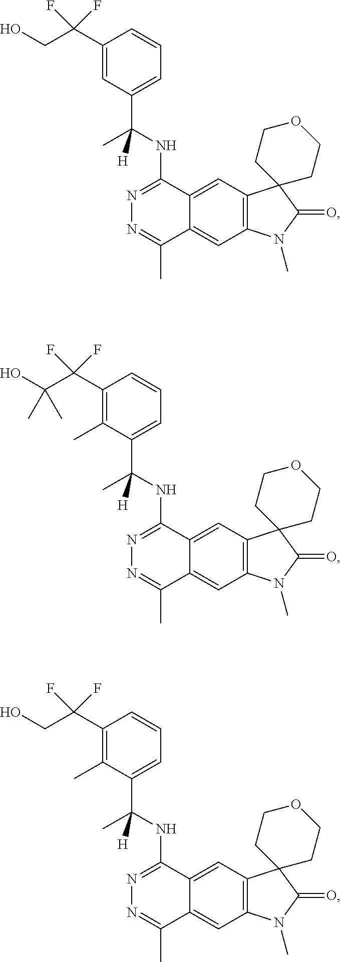 [SMILES: Cc1nnc(N[C@H](C)c2cccc(C(F)(F)CO)c2)c2cc3c(cc12)N(C)C(=O)C31CCOCC1 ; Cc1c([C@@H](C)Nc2nnc(C)c3cc4c(cc23)C2(CCOCC2)C(=O)N4C)cccc1C(F)(F)C(C)(C)O ; Cc1c([C@@H](C)Nc2nnc(C)c3cc4c(cc23)C2(CCOCC2)C(=O)N4C)cccc1C(F)(F)CO]
or a pharmaceutically acceptable salt thereof.

57. A composition comprising the compound of any one of claims 1-56 and a pharmaceutically acceptable excipient.

58. A method of treating cancer, comprising administering to a subject in need thereof a therapeutically effective amount of the compound of any one of claims 1-56, or a pharmaceutically acceptable salt thereof.

59. A method of treating cancer, comprising administering to a subject in need thereof a therapeutically effective amount of the composition of claim 57.

60. The method of claim 58 or 59, wherein the cancer is pancreatic cancer, lung cancer, colorectal cancer, cholangiocarcinoma, multiple myeloma, melanoma, uterine cancer, endometrial cancer, thyroid cancer, acute myeloid leukemia, bladder cancer, urothelial cancer, gastric cancer, cervical cancer, head and neck squamous cell carcinoma, diffuse large B cell lymphoma, oesophageal cancer, chronic lymphocytic leukemia, hepatocellular cancer, breast cancer, ovarian cancer, prostate cancer, glioblastoma, renal cancer or sarcoma.

61. A method of treating a disease associated with or modulated by SOS1, the method comprising administering to a subject a therapeutically effective amount of the compound of any one of claims 1-56, or a pharmaceutically acceptable salt thereof.

62. A method of treating a disease associated with or modulated by SOS1, the method comprising administering to a subject a therapeutically effective amount of the composition of claim 57.

63. The method of claim 61 or 62, wherein treating the disease comprises inhibiting the interaction of SOS1 and a RAS-family protein and/or RAC1.

64. The method of any one of claims 61-63, wherein the disease is Neurofibromatosis type 1 (NF1), Noonan Syndrome (NS), Noonan Syndrome with Multiple Lentigines (NSML; LEOPARD syndrome), Capillary Malformation-Arteriovenous Malformation Syndrome (CM-AVM), Costello Syndrome (CS), Cardio-Facio-Cutaneous Syndrome (CFC), Legius Syndrome (also known as NF1-like Syndrome) or Hereditary gingival fibromatosis.

65. The method of any one of claims 61-63, wherein the disease is pancreatic cancer, lung cancer, colorectal cancer, cholangiocarcinoma, multiple myeloma, melanoma, uterine cancer, endometrial cancer, thyroid cancer, acute myeloid leukemia, bladder cancer, urothelial cancer, gastric cancer, cervical cancer, head and neck squamous cell carcinoma, diffuse large B cell lymphoma, oesophageal cancer, chronic lymphocytic leukemia, hepatocellular cancer, breast cancer, ovarian cancer, prostate cancer, glioblastoma, renal cancer or sarcoma.

66. The method of any one of claims 58-60, wherein the cancer comprises a SOS1 alteration, wherein the SOS1 alteration is SOS1 amplification, SOS1 overexpression, SOS1 mutation, or combination thereof.

67. The method of any one of claims 61-65, wherein the disease comprises a SOS1 alteration, wherein the SOS1 alteration is SOS1 amplification, SOS1 overexpression, SOS1 mutation, or combination thereof.
